(19) United States
(12) Patent Application Publication
(10) Pub. No.: US 2024/0200063 A1
(43) Pub. Date: Jun. 20, 2024

(54) MICROGLIAL GENE SILENCING USING DOUBLE-STRANDED SIRNA

(71) Applicant: Atalanta Therapeutics, Inc., Boston, MA (US)

(72) Inventors: Daniel CURTIS, Belmont, MA (US); Aimee JACKSON, Boston, MA (US); Benjamin ANDREONE, Boston, MA (US); Bruno Miguel DA CRUZ GODINHO, Reading, MA (US); Qingmin CHEN, Lexington, MA (US)

(21) Appl. No.: 18/283,671

(22) PCT Filed: Mar. 24, 2022
(86) PCT No.: PCT/US2022/021789

§ 371 Date: Sep. 22, 2023

Related U.S. Application Data
(60) Provisional application No. 63/165,518, filed on Mar. 24, 2021.

(51) Int. Cl. C12N 15/113

(52) U.S. Cl. CPC C12N 15/113; C12N 2310/14; C12N 2310/315; C12N 2310/321; C12N 2310/322; C12N 2320/32

(57) ABSTRACT

Microglia are an essential part of the immune system in the central nervous system, as well as potential sources of disease. Gene silencing employs short interfering RNA (siRNA) to selectively target genes that are the source of such diseases. By employing branched siRNA, distribution of the siRNA throughout the CNS, including to the resident microglial cells, may be enhanced as compared to unbranched siRNA. Methods and compositions for the use of branched siRNA in a therapy are contained herein.

### BACKGROUND OF THE INVENTION

In many species, introduction of double-stranded RNA (dsRNA) induces potent and specific gene silencing. This phenomenon occurs in both plants and animals and has roles in viral defense and transposon silencing mechanisms. Short interfering RNAs (siRNAs), which are generally much shorter than the target gene, have been shown to be effective at gene silencing.

Microglia are a type of glial cell found in the central nervous system (CNS). Microglia are an essential component of the CNS immune system; however, microglia with dysregulated genes can also be a source of disease. For example, a disease state may precipitate as a result of overactive microglial genes or genes with reduced expression and/or activity in microglia. Therefore, silencing of effector genes or pathway regulatory genes may be needed to restore normal gene network function and ameliorate the disease state. Thus, there remains a need for new and improved therapeutics capable of permeating microglial cells and silencing microglial genes in order to restore genetic and biochemical pathway activity in microglia from a disease state towards a normal healthy state.

### SUMMARY OF THE INVENTION

In an aspect, the invention features a method of delivering a branched small interfering RNA (siRNA) molecule to a microglial cell in a subject in need of microglial gene silencing. The method may include administering the branched siRNA molecule to the subject (e.g., to the central nervous system of the subject).

In some embodiments, the subject has been diagnosed as having a disease associated with expression of a dysregulated microglial gene or dysregulated microglial gene pathway. In some embodiments, the subject has been diagnosed as having a disease associated with expression and/or activity of a dysregulated microglial gene (e.g., altered expression and/or activity of a wild-type or mutated microglial gene).

In some embodiments, the dysregulated microglial gene exhibits increased expression and/or activity in microglial cells of the subject as compared to the expression and/or activity of the microglial gene in microglial cells of a reference subject. In some embodiments, the dysregulated microglial gene exhibits reduced expression and/or activity in microglial cells of the subject as compared to the expression and/or activity of the microglial gene in microglial cells of a reference subject.

In some embodiments, the microglial gene is a positive regulator of a gene for which increased expression and/or activity relative to the level of expression and/or activity observed in a reference subject is associated with a disease state.

In some embodiments, the microglial gene is a negative regulator of a gene for which decreased expression and/or activity relative to the level of expression and/or activity observed in a reference subject is associated with a disease state.

In some embodiments, the microglial gene is a splice isoform of a gene for which overexpression of the splice isoform relative to the expression of the splice isoform in a reference subject is associated with a disease state.

In some embodiments, the disease is a neuroinflammatory disease or a neurodegenerative disease. In some embodiments, the disease is Alzheimer's disease. In some embodiments, the disease is Amyotrophic Lateral Sclerosis. In some embodiments, the disease is Parkinson's disease. In some embodiments, the disease is frontotemporal dementia. In some embodiments, the disease is Huntington's disease. In some embodiments, the disease is multiple sclerosis. In some embodiments, the disease is progressive supranuclear palsy.

In some embodiments, the dysregulated microglial gene is selected from the group consisting of ABCA7, ABI3, ADAM10, APOC1, APOE, AXL, BIN1, C1QA, C3, C9ORF72, CASS4, CCL5, CD2AP, CD33, CD68, CLPTM1, CLU, CR1, CSF1, CST7, CTSB, CTSD, CTSL, CXCL10, CXCL13, DSG2, ECHDC3, EPHA1, FABP5, FERMT2, FTH1, GNAS, GRN, HBEGF, HLA-DRB1, HLA-DRB5, IFIT1, IFIT3, IFITM3, IFNAR1, IFNAR2, IGF1, IL10RA, ILIA, IL1B, IL1RAP, INPP5D, ITGAM, ITGAX, LILRB4, LPL, MEF2C, MMP12, MS4A4A, MS4A6A, NLRP3, NME8, NOS2, PICALM, PILRA, PLCG2, PTK2B, SCIMP, SLC24A4, SORL1, SPI1, SPP1, SPPL2A, TBK1, TNF, TREM2, TREML2, TYROBP, and ZCWPW1.

In some embodiments, the subject is a mammal (e.g., a human).

In some embodiments, the branched siRNA is administered to the subject intrathecally, intracerebroventricularly, or intrastriatally.

In some embodiments, the siRNA molecule is di-branched. In some embodiments, the siRNA molecule is tri-branched. In some embodiments, the siRNA molecule is tetra-branched.

In some embodiments, the siRNA comprises (i) an antisense strand having complementarity to a portion of one or more of genes selected from the group consisting of APOE, BIN1, C1QA, C3, C9ORF72, CCL5, CD33, CLU/APOJ, CR1, CXCL10, CXCL13, IFIT1, IFIT3, IFITM3, IFNAR1, IFNAR2, IL10RA, ILIA, IL1B, IL1RAP, INPP5D, ITGAM, MEF2C, MMP12, NLRP3, NOS2, PILRA, PLCG2, PTK2B, SLC24A4, TBK1, and TNF and (ii) a sense strand having complementarity to the antisense strand.

In some embodiments, the siRNA includes (i) an antisense strand having complementarity to a portion of a gene encoding a positive regulator of a gene for which increased expression and/or activity (relative, e.g., to the level of expression and/or activity observed in a reference subject) is associated with a disease state.

In some embodiments, the siRNA includes (i) an antisense strand having complementarity to a portion of a gene encoding a negative regulator of a gene for which decreased expression and/or activity (relative, e.g., to the level of expression and/or activity observed in a reference subject) is associated with a disease state.

In some embodiments, the siRNA includes (i) an antisense strand having complementarity to a splice isoform of a gene for which overexpression of the splice isoform relative to the expression of the splice isoform in a reference subject is associated with a disease state.

In any of the foregoing embodiments, the siRNA may also include (ii) a sense strand having complementarity to the antisense strand.

In some embodiment, the antisense strand has complementarity (e.g., at least 85% complementarity, such as 85% complementarity, 86% complementarity, 87% complementarity, 88% complementarity, 89% complementarity, 90% complementarity, 91% complementarity, 92% complementarity, 93% complementarity, 94% complementarity, 95% complementarity, 96% complementarity, 97% complementarity, 98% complementarity, 99% complementarity, or 100% complementarity) to a portion of at least 10 contiguous nucleotides of an mRNA molecule encoding one or more of the above genes. For example, the antisense strand may have complementarity to a portion of 10 contiguous nucleotides, 11 contiguous nucleotides, 12 contiguous nucleotides, 13 contiguous nucleotides, 14 contiguous nucleotides, 15 contiguous nucleotides, 16 contiguous nucleotides, 17 contiguous nucleotides, 18 contiguous nucleotides, 19 contiguous nucleotides, 20 contiguous nucleotides, 21 contiguous nucleotides, 22 contiguous nucleotides, 23 contiguous nucleotides, 24 contiguous nucleotides, 25 contiguous nucleotides, 26 contiguous nucleotides, 27 contiguous nucleotides, 28 contiguous nucleotides, 29 contiguous nucleotides, 30 contiguous nucleotides, 31 contiguous nucleotides, 32 contiguous nucleotides 33 contiguous nucleotides, 34 contiguous nucleotides, contiguous nucleotides, 36 contiguous nucleotides, 37 contiguous nucleotides, 38 contiguous nucleotides, 39 contiguous nucleotides, 40 contiguous nucleotides, 41 contiguous nucleotides, 42 contiguous nucleotides, 43 contiguous nucleotides, 44 contiguous nucleotides, 45 contiguous nucleotides, 46 contiguous nucleotides, 47 contiguous nucleotides, 48 contiguous nucleotides, 49 contiguous nucleotides, or 50 contiguous nucleotides, or more, of an mRNA molecule encoding one or more of the above genes.

In some embodiments, the antisense strand has complementarity (e.g., at least 85% complementarity, such as 85% complementarity, 86% complementarity, 87% complementarity, 88% complementarity, 89% complementarity, 90% complementarity, 91% complementarity, 92% complementarity, 93% complementarity, 94% complementarity, 95% complementarity, 96% complementarity, 97% complementarity, 98% complementarity, 99% complementarity, or 100% complementarity) to a portion of from 10 to 50 contiguous nucleotides of an mRNA molecule encoding one or more of the above genes. For example, the antisense strand may have complementarity to a portion of from 11 contiguous nucleotides to 45 contiguous nucleotides, from 12 contiguous nucleotides to contiguous nucleotides, from 13 contiguous nucleotides to 35 contiguous nucleotides, from 14 contiguous nucleotides to 30 contiguous nucleotides, from 15 contiguous nucleotides to 29 contiguous nucleotides, from 16 contiguous nucleotides to 28 contiguous nucleotides, from 17 contiguous nucleotides to 27 contiguous nucleotides, from 18 contiguous nucleotides to 26 contiguous nucleotides, or from 19 contiguous nucleotides to 22 contiguous nucleotides of an mRNA molecule encoding one or more of the above genes.

In some embodiments, the antisense strand comprises a region represented by the following chemical formula, in the 5′-to-3′ direction:

Z-((A-P-)ₙ(B-P-)ₘ)_{q};

wherein Z is a 5′ phosphorus stabilizing moiety; each A is, independently, a 2′-O-methyl (2′-O-Me) ribonucleoside; each B is, independently, a 2′-fluoro-ribonucleoside; each P is, independently, an internucleoside linkage selected from a phosphodiester linkage and a phosphorothioate linkage; n is an integer from 1 to 5 (e.g., 1, 2, 3, 4, or 5); m is an integer from 1 to 5 (e.g., 1, 2, 3, 4, or 5); and q is an integer between 1 and 15 (1, 2, 3, 4, 5, 6, 7, 8, 9, 10, 11, 12, 13, 14, or 15).

In some embodiments, the antisense strand has a structure represented by Formula A-I, wherein Formula A-I is, in the 5′-to-3′ direction:

A-B-(A′)ⱼ-C-P²-D-P¹-(C′-P¹)ₖ-C′ Formula A-I;
wherein A is represented by the formula C-P¹-D-P¹;
each A′ is represented by the formula C-P²-D-P²;
B is represented by the formula C-P²-D-P²-D-P²-D-P²;
each C is a 2′-O-methyl (2′-O-Me) ribonucleoside;
each C′, independently, is a 2′-O-Me ribonucleoside or a 2′-fluoro (2′-F) ribonucleoside;
each D is a 2′-F ribonucleoside;
each P¹ is a phosphorothioate internucleoside linkage;
each P² is a phosphodiester internucleoside linkage;
j is an integer from 1 to 7 (e.g., 1, 2, 3, 4, 5, 6, or 7); and
k is an integer from 1 to 7 (e.g., 1, 2, 3, 4, 5, 6, or 7).

In some embodiments, the antisense strand has a structure represented by Formula A1, wherein Formula A1 is, in the 5′-to-3′ direction:

A-S-B-S-A-O-B-O-B-O-B-O-A-O-B-O-A-O-B-O-A-O-B-O-A-O-B-O-A-O-B-S-A-S-A-S-A-S-B-S-A Formula A1;

wherein A represents a 2′-O-Me ribonucleoside, B represents a 2′-F ribonucleoside, 0 represents a phosphodiester internucleoside linkage, and S represents a phosphorothioate internucleoside linkage.

In some embodiments, the antisense strand has a structure represented by Formula A-II, wherein Formula A-II is, in the 5′-to-3′ direction:

A-B-(A′)ⱼ-C-P²-D-P¹-(C-P¹)ₖ-C′ Formula A-I;
wherein A is represented by the formula C-P¹-D-P¹;
each A′ is represented by the formula C-P²-D-P²;
B is represented by the formula C-P²-D-P²-D-P²-D-P²;
each C is a 2′-O-methyl (2′-O-Me) ribonucleoside;
each C′, independently, is a 2′-O-Me ribonucleoside or a 2′-fluoro (2′-F) ribonucleoside;
each D is a 2′-F ribonucleoside;
each P¹ is a phosphorothioate internucleoside linkage;
each P² is a phosphodiester internucleoside linkage;
j is an integer from 1 to 7 (e.g., 1, 2, 3, 4, 5, 6, or 7); and
k is an integer from 1 to 7 (e.g., 1, 2, 3, 4, 5, 6, or 7).

In some embodiments, antisense strand has a structure represented by Formula A2, wherein Formula A2 is, in the 5′-to-3′ direction:

A-S-B-S-A-O-B-O-B-O-B-O-A-O-B-O-A-O-B-O-A-O-B-O-A-O-B-O-A-O-B-S-A-S-A-S-A-S-A-S-A Formula A2;

wherein A represents a 2′-O-Me ribonucleoside, B represents a 2′-F ribonucleoside, 0 represents a phosphodiester internucleoside linkage, and S represents a phosphorothioate internucleoside linkage.

In some embodiments, the sense strand has a structure represented by Formula S-III, wherein Formula S-III is, in the 5′-to-3′ direction:

E-(A′)ₘ-F Formula S-III;
wherein E is represented by the formula (C-P¹)₂;
F is represented by the formula (C-P²)₃-D-P¹-C-P¹-C, (C-P²)₃-D-P²-C-P²-C, (C-P²)₃-D-P¹-C-P¹-D, or (C-P²)₃-D-P²-C-P²-D;
A′, C, D, P¹, and P² are as defined in Formula II; and
m is an integer from 1 to 7 (e.g., 1, 2, 3, 4, 5, 6, or 7).

In some embodiments, the sense strand has a structure represented by Formula S1, wherein Formula S1 is, in the 5′-to-3′ direction:

A-S-A-S-A-O-B-O-A-O-B-O-A-O-B-O-A-O-B-O-A-O-A-O-A-O-B-S-A-S-A Formula S1;

wherein A represents a 2′-O-Me ribonucleoside, B represents a 2′-F ribonucleoside, 0 represents a phosphodiester internucleoside linkage, and S represents a phosphorothioate internucleoside linkage.

In some embodiments, the sense strand has a structure represented by Formula S2, wherein Formula S2 is, in the 5′-to-3′ direction:

A-S-A-S-A-O-B-O-A-O-B-O-A-O-B-O-A-O-B-O-A-O-A-O-A-O-B-O-A-O-A Formula S2;

wherein A represents a 2′-O-Me ribonucleoside, B represents a 2′-F ribonucleoside, 0 represents a phosphodiester internucleoside linkage, and S represents a phosphorothioate internucleoside linkage.

In some embodiments, the sense strand has a structure represented by Formula S3, wherein Formula S3 is, in the 5′-to-3′ direction:

A-S-A-S-A-O-B-O-A-O-B-O-A-O-B-O-A-O-B-O-A-O-A-O-A-O-B-S-A-S-B Formula S3;

wherein A represents a 2′-O-Me ribonucleoside, B represents a 2′-F ribonucleoside, 0 represents a phosphodiester internucleoside linkage, and S represents a phosphorothioate internucleoside linkage.

In some embodiments, the sense strand has a structure represented by Formula S4, wherein Formula S4 is, in the 5′-to-3′ direction:

A-S-A-S-A-O-B-O-A-O-B-O-A-O-B-O-A-O-B-O-A-O-A-O-A-O-B-O-A-O-B Formula S4;

wherein A represents a 2′-O-Me ribonucleoside, B represents a 2′-F ribonucleoside, 0 represents a phosphodiester internucleoside linkage, and S represents a phosphorothioate internucleoside linkage.

In some embodiments, the antisense strand has a structure represented by Formula A-IV, wherein Formula A-IV is, in the 5′-to-3′ direction:

A-(A′)ⱼ-C-P²-B-(C-P¹)ₖ-C′ Formula A-IV;
wherein A is represented by the formula C-P¹-D-P¹;
each A′ is represented by the formula C-P²-D-P²;
B is represented by the formula D-P¹-C-P¹-D-P¹;
each C is a 2′-O-Me ribonucleoside;
each C′, independently, is a 2′-O-Me ribonucleoside or a 2′-F ribonucleoside;
each D is a 2′-F ribonucleoside;
each P¹ is a phosphorothioate internucleoside linkage;
each P² is a phosphodiester internucleoside linkage;
j is an integer from 1 to 7 (e.g., 1, 2, 3, 4, 5, 6, or 7); and
k is an integer from 1 to 7 (e.g., 1, 2, 3, 4, 5, 6, or 7).

In some embodiments, the antisense strand has a structure represented by Formula A3, wherein Formula A3 is, in the 5′-to-3′ direction:

A-S-B-S-A-O-B-O-A-O-B-O-A-O-B-O-A-O-B-O-A-O-B-O-A-O-B-O-A-O-B-S-A-S-B-S-A-S-A-S-A Formula A3;

wherein A represents a 2′-O-Me ribonucleoside, B represents a 2′-F ribonucleoside, 0 represents a phosphodiester internucleoside linkage, and S represents a phosphorothioate internucleoside linkage.

In some embodiments, the sense strand has a structure represented by Formula S-V, wherein Formula S-V is, in the 5′-to-3′ direction:

E-(A′)ₘ-C-P²-F Formula S-V;
wherein E is represented by the formula (C-P¹)₂;
F is represented by the formula D-P¹-C-P¹-C, D-P²-C-P²-C, D-P¹-C-P¹-D, or D-P²-C-P²-D;
A′, C, D, P¹ and P² are as defined in Formula IV; and
m is an integer from 1 to 7 (e.g., 1, 2, 3, 4, 5, 6, or 7).

In some embodiments, the sense strand has a structure represented by Formula S5, wherein Formula S5 is, in the 5′-to-3′ direction:

A-S-A-S-A-O-B-O-A-O-B-O-A-O-B-O-A-O-B-O-A-O-B-O-A-O-B-S-A-S-A Formula S5;

wherein A represents a 2′-O-Me ribonucleoside, B represents a 2′-F ribonucleoside, 0 represents a phosphodiester internucleoside linkage, and S represents a phosphorothioate internucleoside linkage.

In some embodiments, the sense strand has a structure represented by Formula S6, wherein Formula S6 is, in the 5′-to-3′ direction:

A-S-A-S-A-O-B-O-A-O-B-O-A-O-B-O-A-O-B-O-A-O-B-O-A-O-B-O-A-O-A Formula S6;

wherein A represents a 2′-O-Me ribonucleoside, B represents a 2′-F ribonucleoside, 0 represents a phosphodiester internucleoside linkage, and S represents a phosphorothioate internucleoside linkage.

In some embodiments, the sense strand has a structure represented by Formula S7, wherein Formula S7 is, in the 5′-to-3′ direction:

A-S-A-S-A-O-B-O-A-O-B-O-A-O-B-O-A-O-B-O-A-O-B-O-A-O-B-S-A-S-B Formula S7;

wherein A represents a 2′-O-Me ribonucleoside, B represents a 2′-F ribonucleoside, 0 represents a phosphodiester internucleoside linkage, and S represents a phosphorothioate internucleoside linkage.

In some embodiments, the sense strand has a structure represented by Formula S8, wherein Formula S8 is, in the 5′-to-3′ direction:

A-S-A-S-A-O-B-O-A-O-B-O-A-O-B-O-A-O-B-O-A-O-B-O-A-O-B-O-A-O-B Formula S8;

wherein A represents a 2′-O-Me ribonucleoside, B represents a 2′-F ribonucleoside, 0 represents a phosphodiester internucleoside linkage, and S represents a phosphorothioate internucleoside linkage.

In some embodiments, the antisense strand has a structure represented by Formula A-VI, wherein Formula A-VI is, in the 5′-to-3′ direction:

A-Bⱼ-E-Bₖ-E-F-Gₗ-D-P¹-C′ Formula A-VI;
wherein A is represented by the formula C-P¹-D-P¹;
each B is represented by the formula C-P²;
each C is a 2′-O-Me ribonucleoside;
each C′, independently, is a 2′-O-Me ribonucleoside or a 2′-F ribonucleoside;
each D is a 2′-F ribonucleoside;
each E is represented by the formula D-P²-C-P²;
F is represented by the formula D-P¹-C-P¹;
each G is represented by the formula C-P¹;
each P¹ is a phosphorothioate internucleoside linkage;
each P² is a phosphodiester internucleoside linkage;
j is an integer from 1 to 7 (e.g., 1, 2, 3, 4, 5, 6, or 7);
k is an integer from 1 to 7 (e.g., 1, 2, 3, 4, 5, 6, or 7); and
I is an integer from 1 to 7 (e.g., 1, 2, 3, 4, 5, 6, or 7).

In some embodiments, the antisense strand has a structure represented by Formula A4, wherein Formula A4 is, in the 5′-to-3′ direction:

A-S-B-S-A-O-A-O-A-O-B-O-A-O-A-O-A-O-A-O-A-O-A-O-A-O-B-O-A-O-B-S-A-S-A-S-A-S-B-S-A Formula A4;

wherein A represents a 2′-O-Me ribonucleoside, B represents a 2′-F ribonucleoside, 0 represents a phosphodiester internucleoside linkage, and S represents a phosphorothioate internucleoside linkage.

In some embodiments, the sense strand has a structure represented by Formula S-VII, wherein Formula S-VII is, in the 5′-to-3′ direction:

H-Bₘ-Iₙ-A′-Bₒ-H-C Formula S-VII;
wherein A′ is represented by the formula C-P²-D-P²;
each H is represented by the formula (C-P¹)₂;
each I is represented by the formula (D-P²);
B, C, D, P¹ and P² are as defined in Formula VI;
m is an integer from 1 to 7 (e.g., 1, 2, 3, 4, 5, 6, or 7);
n is an integer from 1 to 7 (e.g., 1, 2, 3, 4, 5, 6, or 7); and
o is an integer from 1 to 7 (e.g., 1, 2, 3, 4, 5, 6, or 7).

In some embodiments, the sense strand has a structure represented by Formula S9, wherein Formula S9 is, in the 5′-to-3′ direction:

A-S-A-S-A-O-A-O-A-O-B-O-B-O-B-O-A-O-B-O-A-O-A-O-A-O-A-S-A-S-A Formula S9;

wherein A represents a 2′-O-Me ribonucleoside, B represents a 2′-F ribonucleoside, 0 represents a phosphodiester internucleoside linkage, and S represents a phosphorothioate internucleoside linkage.

In some embodiments, the antisense strand also has a 5′ phosphorus stabilizing moiety at the 5′ end of the antisense strand.

In some embodiments, the sense strand also has a 5′ phosphorus stabilizing moiety at the 5′ end of the sense strand.

In some embodiments, each 5′-phosphorus stabilizing moiety is, independently represented by any one of Formula I-VIII:

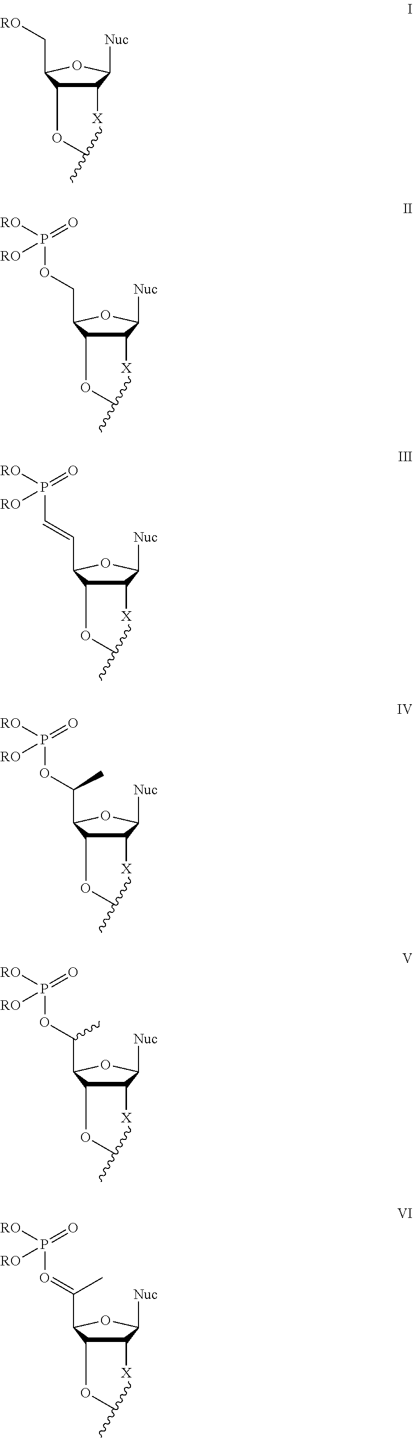

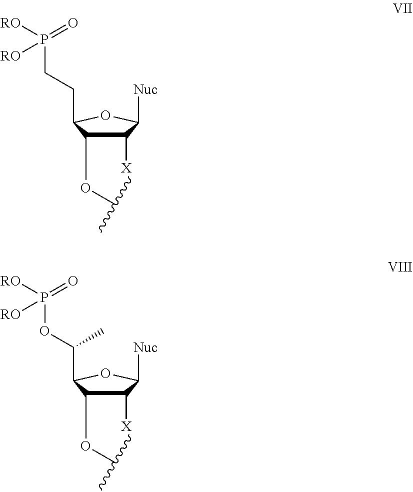

wherein Nuc represents a nucleobase, such as adenine, uracil, guanine, thymine, or cytosine, and R represents optionally substituted alkyl, optionally substituted alkenyl, or optionally substituted alkynyl (e.g., optionally substituted C1-C6 alkyl, optionally substituted C2-C6 alkenyl, or optionally substituted C2-C6 alkynyl), phenyl, benzyl, hydroxy, or hydrogen.

In some embodiments, Z is (E)-vinylphosphonate as represented in Formula III.

In some embodiments, n is from 1 to 4. In some embodiments, n is from 1 to 3. In some embodiments, n is from 1 to 2. In some embodiments, n is 1.

In some embodiments, m is from 1 to 4. In some embodiments, m is from 1 to 3. In some embodiments, m is from 1 to 2. In some embodiments, m is 1.

In some embodiments, n and m are each 1.

In some embodiments, 50% or more of the ribonucleotides in the antisense strand are 2′-O-Me ribonucleotides (e.g., 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of the ribonucleotides in the antisense strand may be 2′-O-Me ribonucleotides).

In some embodiments, 60% or more of the ribonucleotides in the antisense strand are 2′-O-Me ribonucleotides (e.g., 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of the ribonucleotides in the antisense strand may be 2′-O-Me ribonucleotides).

In some embodiments, 70% or more of the ribonucleotides in the antisense strand are 2′-O-Me ribonucleotides (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of the ribonucleotides in the antisense strand may be 2′-O-Me ribonucleotides).

In some embodiments, 80% or more of the ribonucleotides in the antisense strand are 2′-O-Me ribonucleotides (e.g., 80%, 81%, 82%, 83%, 84%, 85%, 86%, 87%, 88%, 89%, 90%, 91%, 92%, 93%, 94%, 95%, 96%, 97%, 98%, 99%, or 100% of the ribonucleotides in the antisense strand may be 2′-O-Me ribonucleotides).

In some embodiments, 90% or more of the ribonucleotides in the antisense strand are 2′-O-Me ribonucleotides (e.g., 90%, 91%, 92%, 93%, 94%, 95%, 96%, 97%, 98%, 99%, or 100% of the ribonucleotides in the antisense strand may be 2′-O-Me ribonucleotides).

In some embodiments, 10% or less of the internucleoside linkages are phosphodiester linkages or phosphorothioate linkages. In some embodiments, at least 10%, 20%, 30%, 40%, 50%, 60%, 70%, 80%, or 90% of the internucleoside linkages are phosphodiester linkages or phosphorothioate linkages.

In some embodiments, 100% of the internucleoside linkages are phosphodiester linkages or phosphorothioate linkages.

In some embodiments, 9 internucleoside linkages are phosphodiester linkages or phosphorothioate linkages.

In some embodiments, the length of the antisense strand is between 10 and 30 nucleotides (e.g., nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, or 30 nucleotides), 15 and 25 nucleotides (e.g., 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, or 25 nucleotides), or 18 and 23 nucleotides (e.g., 18 nucleotides, 19 nucleotides, 20 nucleotides, 21 nucleotides, 22 nucleotides, or 23 nucleotides). In some embodiments, the length of the antisense strand is 20 nucleotides. In some embodiments, the length of the antisense strand is 21 nucleotides. In some embodiments, the length of the antisense strand is 22 nucleotides. In some embodiments, the length of the antisense strand is 23 nucleotides. In some embodiments, the length of the antisense strand is 24 nucleotides. In some embodiments, the length of the antisense strand is 25 nucleotides. In some embodiments, the length of the antisense strand is 26 nucleotides. In some embodiments, the length of the antisense strand is 27 nucleotides. In some embodiments, the length of the antisense strand is 28 nucleotides. In some embodiments, the length of the antisense strand is 29 nucleotides. In some embodiments, the length of the antisense strand is 30 nucleotides.

In some embodiments, the siRNA molecules of the branched compound are joined to one another by way of a linker (e.g., an ethylene glycol oligomer, such as tetraethylene glycol). In some embodiments, the siRNA molecules of the branched compound are joined to one another by way of a linker between the sense strand of one siRNA molecule and the sense strand of the other siRNA molecule. In some embodiments, the siRNA molecules are joined by way of linkers between the antisense strand of one siRNA molecule and the antisense strand of the other siRNA molecule. In some embodiments, the siRNA molecules of the branched compound are joined to one another by way of a linker between the sense strand of one siRNA molecule and the antisense strand of the other siRNA molecule.

In some embodiments, the length of the sense strand is between 12 and 30 nucleotides (e.g., 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, or 30 nucleotides), or 14 and 18 nucleotides (e.g., 14 nucleotides, 15 nucleotides, 16 nucleotides, 17 nucleotides, or 18 nucleotides). In some embodiments, the length of the sense strand is 15 nucleotides. In some embodiments, the length of the sense strand is 16 nucleotides. In some embodiments, the length of the sense strand is 17 nucleotides. In some embodiments, the length of the sense strand is 18 nucleotides. In some embodiments, the length of the sense strand is 19 nucleotides. In some embodiments, the length of the sense strand is 20 nucleotides. In some embodiments, the length of the sense strand is 21 nucleotides. In some embodiments, the length of the sense strand is 22 nucleotides. In some embodiments, the length of the sense strand is 23 nucleotides. In some embodiments, the length of the sense strand is 24 nucleotides. In some embodiments, the length of the sense strand is 25 nucleotides. In some embodiments, the length of the sense strand is 26 nucleotides. In some embodiments, the length of the sense strand is 27 nucleotides. In some embodiments, the length of the sense strand is 28 nucleotides. In some embodiments, the length of the sense strand is 29 nucleotides.

In some embodiments, the length of the sense strand is 30 nucleotides.

In some embodiments, 4 internucleoside linkages are phosphorothioate linkages.

In some embodiments, the antisense strand is 18 nucleotides in length and the sense strand is 14 nucleotides in length.

In some embodiments, the antisense strand is 18 nucleotides in length and the sense strand is nucleotides in length.

In some embodiments, the antisense strand is 18 nucleotides in length and the sense strand is 16 nucleotides in length.

In some embodiments, the antisense strand is 18 nucleotides in length and the sense strand is 17 nucleotides in length.

In some embodiments, the antisense strand is 18 nucleotides in length and the sense strand is 18 nucleotides in length.

In some embodiments, the antisense strand is 19 nucleotides in length and the sense strand is 14 nucleotides in length.

In some embodiments, the antisense strand is 19 nucleotides in length and the sense strand is nucleotides in length.

In some embodiments, the antisense strand is 19 nucleotides in length and the sense strand is 16 nucleotides in length.

In some embodiments, the antisense strand is 19 nucleotides in length and the sense strand is 17 nucleotides in length.

In some embodiments, the antisense strand is 19 nucleotides in length and the sense strand is 18 nucleotides in length.

In some embodiments, the antisense strand is 19 nucleotides in length and the sense strand is 19 nucleotides in length.

In some embodiments, the antisense strand is 20 nucleotides in length and the sense strand is 14 nucleotides in length.

In some embodiments, the antisense strand is 20 nucleotides in length and the sense strand is nucleotides in length.

In some embodiments, the antisense strand is 20 nucleotides in length and the sense strand is 16 nucleotides in length.

In some embodiments, the antisense strand is 20 nucleotides in length and the sense strand is 17 nucleotides in length.

In some embodiments, the antisense strand is 20 nucleotides in length and the sense strand is 18 nucleotides in length.

In some embodiments, the antisense strand is 20 nucleotides in length and the sense strand is 19 nucleotides in length.

In some embodiments, the antisense strand is 20 nucleotides in length and the sense strand is nucleotides in length.

In some embodiments, the antisense strand is 21 nucleotides in length and the sense strand is 14 nucleotides in length.

In some embodiments, the antisense strand is 21 nucleotides in length and the sense strand is nucleotides in length.

In some embodiments, the antisense strand is 21 nucleotides in length and the sense strand is 16 nucleotides in length.

In some embodiments, the antisense strand is 21 nucleotides in length and the sense strand is 17 nucleotides in length.

In some embodiments, the antisense strand is 21 nucleotides in length and the sense strand is 18 nucleotides in length.

In some embodiments, the antisense strand is 21 nucleotides in length and the sense strand is 19 nucleotides in length.

In some embodiments, the antisense strand is 21 nucleotides in length and the sense strand is nucleotides in length.

In some embodiments, the antisense strand is 21 nucleotides in length and the sense strand is 21 nucleotides in length.

In some embodiments, the antisense strand is 22 nucleotides in length and the sense strand is 14 nucleotides in length.

In some embodiments, the antisense strand is 22 nucleotides in length and the sense strand is nucleotides in length.

In some embodiments, the antisense strand is 22 nucleotides in length and the sense strand is 16 nucleotides in length.

In some embodiments, the antisense strand is 22 nucleotides in length and the sense strand is 17 nucleotides in length.

In some embodiments, the antisense strand is 22 nucleotides in length and the sense strand is 18 nucleotides in length.

In some embodiments, the antisense strand is 22 nucleotides in length and the sense strand is 19 nucleotides in length.

In some embodiments, the antisense strand is 22 nucleotides in length and the sense strand is nucleotides in length.

In some embodiments, the antisense strand is 22 nucleotides in length and the sense strand is 21 nucleotides in length.

In some embodiments, the antisense strand is 22 nucleotides in length and the sense strand is 22 nucleotides in length.

In some embodiments, the antisense strand is 23 nucleotides in length and the sense strand is 14 nucleotides in length.

In some embodiments, the antisense strand is 23 nucleotides in length and the sense strand is nucleotides in length.

In some embodiments, the antisense strand is 23 nucleotides in length and the sense strand is 16 nucleotides in length.

In some embodiments, the antisense strand is 23 nucleotides in length and the sense strand is 17 nucleotides in length.

In some embodiments, the antisense strand is 23 nucleotides in length and the sense strand is 18 nucleotides in length.

In some embodiments, the antisense strand is 23 nucleotides in length and the sense strand is 19 nucleotides in length.

In some embodiments, the antisense strand is 23 nucleotides in length and the sense strand is nucleotides in length.

In some embodiments, the antisense strand is 23 nucleotides in length and the sense strand is 21 nucleotides in length.

In some embodiments, the antisense strand is 23 nucleotides in length and the sense strand is 22 nucleotides in length.

In some embodiments, the antisense strand is 23 nucleotides in length and the sense strand is 23 nucleotides in length.

In some embodiments, the antisense strand is 24 nucleotides in length and the sense strand is 14 nucleotides in length.

In some embodiments, the antisense strand is 24 nucleotides in length and the sense strand is nucleotides in length.

In some embodiments, the antisense strand is 24 nucleotides in length and the sense strand is 16 nucleotides in length.

In some embodiments, the antisense strand is 24 nucleotides in length and the sense strand is 17 nucleotides in length.

In some embodiments, the antisense strand is 24 nucleotides in length and the sense strand is 18 nucleotides in length.

In some embodiments, the antisense strand is 24 nucleotides in length and the sense strand is 19 nucleotides in length.

In some embodiments, the antisense strand is 24 nucleotides in length and the sense strand is nucleotides in length.

In some embodiments, the antisense strand is 24 nucleotides in length and the sense strand is 21 nucleotides in length.

In some embodiments, the antisense strand is 24 nucleotides in length and the sense strand is 22 nucleotides in length.

In some embodiments, the antisense strand is 24 nucleotides in length and the sense strand is 23 nucleotides in length.

In some embodiments, the antisense strand is 24 nucleotides in length and the sense strand is 24 nucleotides in length.

In some embodiments, the antisense strand is 25 nucleotides in length and the sense strand is 14 nucleotides in length.

In some embodiments, the antisense strand is 25 nucleotides in length and the sense strand is nucleotides in length.

In some embodiments, the antisense strand is 25 nucleotides in length and the sense strand is 16 nucleotides in length.

In some embodiments, the antisense strand is 25 nucleotides in length and the sense strand is 17 nucleotides in length.

In some embodiments, the antisense strand is 25 nucleotides in length and the sense strand is 18 nucleotides in length.

In some embodiments, the antisense strand is 25 nucleotides in length and the sense strand is 19 nucleotides in length.

In some embodiments, the antisense strand is 25 nucleotides in length and the sense strand is nucleotides in length.

In some embodiments, the antisense strand is 25 nucleotides in length and the sense strand is 21 nucleotides in length.

In some embodiments, the antisense strand is 25 nucleotides in length and the sense strand is 22 nucleotides in length.

In some embodiments, the antisense strand is 25 nucleotides in length and the sense strand is 23 nucleotides in length.

In some embodiments, the antisense strand is 25 nucleotides in length and the sense strand is 24 nucleotides in length.

In some embodiments, the antisense strand is 25 nucleotides in length and the sense strand is nucleotides in length.

In some embodiments, the antisense strand is 26 nucleotides in length and the sense strand is 14 nucleotides in length.

In some embodiments, the antisense strand is 26 nucleotides in length and the sense strand is nucleotides in length.

In some embodiments, the antisense strand is 26 nucleotides in length and the sense strand is 16 nucleotides in length.

In some embodiments, the antisense strand is 26 nucleotides in length and the sense strand is 17 nucleotides in length.

In some embodiments, the antisense strand is 26 nucleotides in length and the sense strand is 18 nucleotides in length.

In some embodiments, the antisense strand is 26 nucleotides in length and the sense strand is 19 nucleotides in length.

In some embodiments, the antisense strand is 26 nucleotides in length and the sense strand is nucleotides in length.

In some embodiments, the antisense strand is 26 nucleotides in length and the sense strand is 21 nucleotides in length.

In some embodiments, the antisense strand is 26 nucleotides in length and the sense strand is 22 nucleotides in length.

In some embodiments, the antisense strand is 26 nucleotides in length and the sense strand is 23 nucleotides in length.

In some embodiments, the antisense strand is 26 nucleotides in length and the sense strand is 24 nucleotides in length.

In some embodiments, the antisense strand is 26 nucleotides in length and the sense strand is nucleotides in length.

In some embodiments, the antisense strand is 26 nucleotides in length and the sense strand is 26 nucleotides in length.

In some embodiments, the antisense strand is 27 nucleotides in length and the sense strand is 14 nucleotides in length.

In some embodiments, the antisense strand is 27 nucleotides in length and the sense strand is nucleotides in length.

In some embodiments, the antisense strand is 27 nucleotides in length and the sense strand is 16 nucleotides in length.

In some embodiments, the antisense strand is 27 nucleotides in length and the sense strand is 17 nucleotides in length.

In some embodiments, the antisense strand is 27 nucleotides in length and the sense strand is 18 nucleotides in length.

In some embodiments, the antisense strand is 27 nucleotides in length and the sense strand is 19 nucleotides in length.

In some embodiments, the antisense strand is 27 nucleotides in length and the sense strand is nucleotides in length.

In some embodiments, the antisense strand is 27 nucleotides in length and the sense strand is 21 nucleotides in length.

In some embodiments, the antisense strand is 27 nucleotides in length and the sense strand is 22 nucleotides in length.

In some embodiments, the antisense strand is 27 nucleotides in length and the sense strand is 23 nucleotides in length.

In some embodiments, the antisense strand is 27 nucleotides in length and the sense strand is 24 nucleotides in length.

In some embodiments, the antisense strand is 27 nucleotides in length and the sense strand is nucleotides in length.

In some embodiments, the antisense strand is 27 nucleotides in length and the sense strand is 26 nucleotides in length.

In some embodiments, the antisense strand is 27 nucleotides in length and the sense strand is 27 nucleotides in length.

In some embodiments, the antisense strand is 28 nucleotides in length and the sense strand is 14 nucleotides in length.

In some embodiments, the antisense strand is 28 nucleotides in length and the sense strand is nucleotides in length.

In some embodiments, the antisense strand is 28 nucleotides in length and the sense strand is 16 nucleotides in length.

In some embodiments, the antisense strand is 28 nucleotides in length and the sense strand is 17 nucleotides in length.

In some embodiments, the antisense strand is 28 nucleotides in length and the sense strand is 18 nucleotides in length.

In some embodiments, the antisense strand is 28 nucleotides in length and the sense strand is 19 nucleotides in length.

In some embodiments, the antisense strand is 28 nucleotides in length and the sense strand is nucleotides in length.

In some embodiments, the antisense strand is 28 nucleotides in length and the sense strand is 21 nucleotides in length.

In some embodiments, the antisense strand is 28 nucleotides in length and the sense strand is 22 nucleotides in length.

In some embodiments, the antisense strand is 28 nucleotides in length and the sense strand is 23 nucleotides in length.

In some embodiments, the antisense strand is 28 nucleotides in length and the sense strand is 24 nucleotides in length.

In some embodiments, the antisense strand is 28 nucleotides in length and the sense strand is nucleotides in length.

In some embodiments, the antisense strand is 28 nucleotides in length and the sense strand is 26 nucleotides in length.

In some embodiments, the antisense strand is 28 nucleotides in length and the sense strand is 27 nucleotides in length.

In some embodiments, the antisense strand is 28 nucleotides in length and the sense strand is 28 nucleotides in length.

In some embodiments, the antisense strand is 29 nucleotides in length and the sense strand is 14 nucleotides in length.

In some embodiments, the antisense strand is 29 nucleotides in length and the sense strand is nucleotides in length.

In some embodiments, the antisense strand is 29 nucleotides in length and the sense strand is 16 nucleotides in length.

In some embodiments, the antisense strand is 29 nucleotides in length and the sense strand is 17 nucleotides in length.

In some embodiments, the antisense strand is 29 nucleotides in length and the sense strand is 18 nucleotides in length.

In some embodiments, the antisense strand is 29 nucleotides in length and the sense strand is 19 nucleotides in length.

In some embodiments, the antisense strand is 29 nucleotides in length and the sense strand is nucleotides in length.

In some embodiments, the antisense strand is 29 nucleotides in length and the sense strand is 21 nucleotides in length.

In some embodiments, the antisense strand is 29 nucleotides in length and the sense strand is 22 nucleotides in length.

In some embodiments, the antisense strand is 29 nucleotides in length and the sense strand is 23 nucleotides in length.

In some embodiments, the antisense strand is 29 nucleotides in length and the sense strand is 24 nucleotides in length.

In some embodiments, the antisense strand is 29 nucleotides in length and the sense strand is nucleotides in length.

In some embodiments, the antisense strand is 29 nucleotides in length and the sense strand is 26 nucleotides in length.

In some embodiments, the antisense strand is 29 nucleotides in length and the sense strand is 27 nucleotides in length.

In some embodiments, the antisense strand is 29 nucleotides in length and the sense strand is 28 nucleotides in length.

In some embodiments, the antisense strand is 29 nucleotides in length and the sense strand is 29 nucleotides in length.

In some embodiments, the antisense strand is 30 nucleotides in length and the sense strand is 14 nucleotides in length.

In some embodiments, the antisense strand is 30 nucleotides in length and the sense strand is nucleotides in length.

In some embodiments, the antisense strand is 30 nucleotides in length and the sense strand is 16 nucleotides in length.

In some embodiments, the antisense strand is 30 nucleotides in length and the sense strand is 17 nucleotides in length.

In some embodiments, the antisense strand is 30 nucleotides in length and the sense strand is 18 nucleotides in length.

In some embodiments, the antisense strand is 30 nucleotides in length and the sense strand is 19 nucleotides in length.

In some embodiments, the antisense strand is 30 nucleotides in length and the sense strand is nucleotides in length.

In some embodiments, the antisense strand is 30 nucleotides in length and the sense strand is 21 nucleotides in length.

In some embodiments, the antisense strand is 30 nucleotides in length and the sense strand is 22 nucleotides in length.

In some embodiments, the antisense strand is 30 nucleotides in length and the sense strand is 23 nucleotides in length.

In some embodiments, the antisense strand is 30 nucleotides in length and the sense strand is 24 nucleotides in length.

In some embodiments, the antisense strand is 30 nucleotides in length and the sense strand is nucleotides in length.

In some embodiments, the antisense strand is 30 nucleotides in length and the sense strand is 26 nucleotides in length.

In some embodiments, the antisense strand is 30 nucleotides in length and the sense strand is 27 nucleotides in length.

In some embodiments, the antisense strand is 30 nucleotides in length and the sense strand is 28 nucleotides in length.

In some embodiments, the antisense strand is 30 nucleotides in length and the sense strand is 29 nucleotides in length.

In some embodiments, the antisense strand is 30 nucleotides in length and the sense strand is nucleotides in length.

In another aspect, the invention features a branched siRNA molecule including a sense strand and an antisense strand, wherein the antisense strand includes a region having complementarity to a segment of contiguous nucleotides within a gene selected from the group consisting of APOE, BIN1, C1QA, C3, C9ORF72, CCL5, CD33, CLU/APOJ, CR1, CXCL10, CXCL13, IFIT1, IFIT3, IFITM3, IFNAR1, IFNAR2, IL10RA, ILIA, IL1B, IL1RAP, INPP5D, ITGAM, MEF2C, MMP12, NLRP3, NOS2, PILRA, PLCG2, PTK2B, SLC24A4, TBK1, and TNF.

In some embodiments, the antisense strand has complementarity to a portion of a gene encoding a positive regulator of a gene for which increased expression and/or activity relative to the level of expression and/or activity observed in a reference subject is associated with a disease state.

In some embodiments, the antisense strand has complementarity to a portion of a gene encoding a negative regulator of a gene for which decreased expression and/or activity relative to the level of expression and/or activity observed in a reference subject is associated with a disease state.

In some embodiments, the antisense strand has complementarity to a splice isoform of a gene for which overexpression of the splice isoform relative to the expression of the splice isoform in a reference subject is associated with a disease state.

In some embodiments, the sense strand has complementarity to the antisense strand.

In some embodiments, the siRNA molecule is di-branched. In some embodiments, the siRNA molecule is tri-branched. In some embodiments, the siRNA molecule is tetra-branched.

In some embodiments, the antisense strand of the branched siRNA has the following Formula in the 5′-to-3′ direction:

Z-((A-P-)ₙ(B-P-)ₘ)_{q};

wherein Z is a 5′ phosphorus stabilizing moiety; each A is, independently, a 2′-O-Me ribonucleoside; each B is, independently, a 2′-fluoro-ribonucleoside; each P is, independently, an internucleoside linkage selected from a phosphodiester linkage and a phosphorothioate linkage; n is an integer from 1 to 5 (e.g., 1, 2, 3, 4, or 5); m is an integer from 1 to 5 (e.g., 1, 2, 3, 4, or 5); and q is an integer between 1 and 15 (e.g., 1, 2, 3, 4, 5, 6, 7, 8, 9, 10, 11, 12, 13, 14, or 15).

In some embodiments, the antisense strand has a structure represented by Formula A-I, wherein Formula A-I is, in the 5′-to-3′ direction:

A-B-(A′)ⱼ-C-P²-D-P¹-(C′-P¹)ₖ-C′ Formula A-I;
wherein A is represented by the formula C-P¹-D-P¹;
each A′ is represented by the formula C-P²-D-P²;
B is represented by the formula C-P²-D-P²-D-P²-D-P²;
each C is a 2′-O-methyl (2′-O-Me) ribonucleoside;
each C′, independently, is a 2′-O-Me ribonucleoside or a 2′-fluoro (2′-F) ribonucleoside;
each D is a 2′-F ribonucleoside;
each P¹ is a phosphorothioate internucleoside linkage;
each P² is a phosphodiester internucleoside linkage;
j is an integer from 1 to 7 (e.g., 1, 2, 3, 4, 5, 6, or 7); and
k is an integer from 1 to 7 (e.g., 1, 2, 3, 4, 5, 6, or 7).

In some embodiments, the antisense strand has a structure represented by Formula A1, wherein Formula A1 is, in the 5′-to-3′ direction:

A-S-B-S-A-O-B-O-B-O-B-O-A-O-B-O-A-O-B-O-A-O-B-O-A-O-B-O-A-O-B-S-A-S-A-S-A-S-B-S-A Formula A1;

wherein A represents a 2′-O-Me ribonucleoside, B represents a 2′-F ribonucleoside, 0 represents a phosphodiester internucleoside linkage, and S represents a phosphorothioate internucleoside linkage.

In some embodiments, the antisense strand has a structure represented by Formula A-II, wherein Formula A-II is, in the 5′-to-3′ direction:

A-B-(A′)ⱼ-C-P²-D-P¹-(C-P¹)ₖ-C′ Formula A-I;
wherein A is represented by the formula C-P¹-D-P¹;
each A′ is represented by the formula C-P²-D-P²;
B is represented by the formula C-P²-D-P²-D-P²-D-P²;
each C is a 2′-O-methyl (2′-O-Me) ribonucleoside;
each C′, independently, is a 2′-O-Me ribonucleoside or a 2′-fluoro (2′-F) ribonucleoside;
each D is a 2′-F ribonucleoside;
each P¹ is a phosphorothioate internucleoside linkage;
each P² is a phosphodiester internucleoside linkage;
j is an integer from 1 to 7 (e.g., 1, 2, 3, 4, 5, 6, or 7); and
k is an integer from 1 to 7 (e.g., 1, 2, 3, 4, 5, 6, or 7).

In some embodiments, antisense strand has a structure represented by Formula A2, wherein Formula A2 is, in the 5′-to-3′ direction:

A-S-B-S-A-O-B-O-B-O-B-O-A-O-B-O-A-O-B-O-A-O-B-O-A-O-B-O-A-O-B-S-A-S-A-S-A-S-A-S-A Formula A2;

wherein A represents a 2′-O-Me ribonucleoside, B represents a 2′-F ribonucleoside, 0 represents a phosphodiester internucleoside linkage, and S represents a phosphorothioate internucleoside linkage.

In some embodiments, the sense strand has a structure represented by Formula S-III, wherein Formula S-III is, in the 5′-to-3′ direction:

E-(A′)ₘ-F Formula S-III;
wherein E is represented by the formula (C-P¹)₂;
F is represented by the formula (C-P²)₃-D-P¹-C-P¹-C, (C-P²)₃-D-P²-C-P²-C, (C-P²)₃-D-P¹-C-P¹-D, or (C-P²)₃-D-P²-C-P²-D;
A′, C, D, P¹, and P² are as defined in Formula II; and
m is an integer from 1 to 7 (e.g., 1, 2, 3, 4, 5, 6, or 7).

In some embodiments, the sense strand has a structure represented by Formula S1, wherein Formula S1 is, in the 5′-to-3′ direction:

A-S-A-S-A-O-B-O-A-O-B-O-A-O-B-O-A-O-B-O-A-O-A-O-A-O-B-S-A-S-A Formula S1;

wherein A represents a 2′-O-Me ribonucleoside, B represents a 2′-F ribonucleoside, 0 represents a phosphodiester internucleoside linkage, and S represents a phosphorothioate internucleoside linkage.

In some embodiments, the sense strand has a structure represented by Formula S2, wherein Formula S2 is, in the 5′-to-3′ direction:

A-S-A-S-A-O-B-O-A-O-B-O-A-O-B-O-A-O-B-O-A-O-A-O-A-O-B-O-A-O-A Formula S2;

wherein A represents a 2′-O-Me ribonucleoside, B represents a 2′-F ribonucleoside, 0 represents a phosphodiester internucleoside linkage, and S represents a phosphorothioate internucleoside linkage.

In some embodiments, the sense strand has a structure represented by Formula S3, wherein Formula S3 is, in the 5′-to-3′ direction:

A-S-A-S-A-O-B-O-A-O-B-O-A-O-B-O-A-O-B-O-A-O-A-O-A-O-B-S-A-S-B Formula S3;

wherein A represents a 2′-O-Me ribonucleoside, B represents a 2′-F ribonucleoside, 0 represents a phosphodiester internucleoside linkage, and S represents a phosphorothioate internucleoside linkage.

In some embodiments, the sense strand has a structure represented by Formula S4, wherein Formula S4 is, in the 5′-to-3′ direction:

A-S-A-S-A-O-B-O-A-O-B-O-A-O-B-O-A-O-B-O-A-O-A-O-A-O-B-O-A-O-B Formula S4;

wherein A represents a 2′-O-Me ribonucleoside, B represents a 2′-F ribonucleoside, 0 represents a phosphodiester internucleoside linkage, and S represents a phosphorothioate internucleoside linkage.

In some embodiments, the antisense strand has a structure represented by Formula A-IV, wherein Formula A-IV is, in the 5′-to-3′ direction:

A-(A′)ⱼ-C-P²-B-(C-P¹)ₖ-C′ Formula A-IV;
wherein A is represented by the formula C-P¹-D-P¹;
each A′ is represented by the formula C-P²-D-P²;
B is represented by the formula D-P¹-C-P¹-D-P¹;
each C is a 2′-O-Me ribonucleoside;
each C′, independently, is a 2′-O-Me ribonucleoside or a 2′-F ribonucleoside;
each D is a 2′-F ribonucleoside;
each P¹ is a phosphorothioate internucleoside linkage;
each P² is a phosphodiester internucleoside linkage;
j is an integer from 1 to 7 (e.g., 1, 2, 3, 4, 5, 6, or 7); and
k is an integer from 1 to 7 (e.g., 1, 2, 3, 4, 5, 6, or 7).

In some embodiments, the antisense strand has a structure represented by Formula A3, wherein Formula A3 is, in the 5′-to-3′ direction:

A-S-B-S-A-O-B-O-A-O-B-O-A-O-B-O-A-O-B-O-A-O-B-O-A-O-B-O-A-O-B-S-A-S-B-S-A-S-A-S-A Formula A3;

wherein A represents a 2′-O-Me ribonucleoside, B represents a 2′-F ribonucleoside, 0 represents a phosphodiester internucleoside linkage, and S represents a phosphorothioate internucleoside linkage.

In some embodiments, the sense strand has a structure represented by Formula S-V, wherein Formula S-V is, in the 5′-to-3′ direction:

E-(A′)ₘ-C-P²-F Formula S-V;
wherein E is represented by the formula (C-P¹)₂;
F is represented by the formula D-P¹-C-P¹-C, D-P²-C-P²-C, D-P¹-C-P¹-D, or D-P²-C-P²-D;
A′, C, D, P¹ and P² are as defined in Formula IV; and
m is an integer from 1 to 7 (e.g., 1, 2, 3, 4, 5, 6, or 7).

In some embodiments, the sense strand has a structure represented by Formula S5, wherein Formula S5 is, in the 5′-to-3′ direction:

A-S-A-S-A-O-B-O-A-O-B-O-A-O-B-O-A-O-B-O-A-O-B-O-A-O-B-S-A-S-A Formula S5;

wherein A represents a 2′-O-Me ribonucleoside, B represents a 2′-F ribonucleoside, 0 represents a phosphodiester internucleoside linkage, and S represents a phosphorothioate internucleoside linkage.

In some embodiments, the sense strand has a structure represented by Formula S6, wherein Formula S6 is, in the 5′-to-3′ direction:

A-S-A-S-A-O-B-O-A-O-B-O-A-O-B-O-A-O-B-O-A-O-B-O-A-O-B-O-A-O-A Formula S6;

wherein A represents a 2′-O-Me ribonucleoside, B represents a 2′-F ribonucleoside, 0 represents a phosphodiester internucleoside linkage, and S represents a phosphorothioate internucleoside linkage.

In some embodiments, the sense strand has a structure represented by Formula S7, wherein Formula S7 is, in the 5′-to-3′ direction:

A-S-A-S-A-O-B-O-A-O-B-O-A-O-B-O-A-O-B-O-A-O-B-O-A-O-B-S-A-S-B Formula S7;

wherein A represents a 2′-O-Me ribonucleoside, B represents a 2′-F ribonucleoside, 0 represents a phosphodiester internucleoside linkage, and S represents a phosphorothioate internucleoside linkage.

In some embodiments, the sense strand has a structure represented by Formula S8, wherein Formula S8 is, in the 5′-to-3′ direction:

A-S-A-S-A-O-B-O-A-O-B-O-A-O-B-O-A-O-B-O-A-O-B-O-A-O-B-O-A-O-B Formula S8;

wherein A represents a 2′-O-Me ribonucleoside, B represents a 2′-F ribonucleoside, 0 represents a phosphodiester internucleoside linkage, and S represents a phosphorothioate internucleoside linkage.

In some embodiments, the antisense strand has a structure represented by Formula A-VI, wherein Formula A-VI is, in the 5′-to-3′ direction:

A-Bⱼ-E-Bₖ-E-F-Gₗ-D-P¹-C′ Formula A-VI;
wherein A is represented by the formula C-P¹-D-P¹;
each B is represented by the formula C-P²;
each C is a 2′-O-Me ribonucleoside;
each C′, independently, is a 2′-O-Me ribonucleoside or a 2′-F ribonucleoside;
each D is a 2′-F ribonucleoside;
each E is represented by the formula D-P²-C-P²;
F is represented by the formula D-P¹-C-P¹;
each G is represented by the formula C-P¹;
each P¹ is a phosphorothioate internucleoside linkage;
each P² is a phosphodiester internucleoside linkage;
j is an integer from 1 to 7 (e.g., 1, 2, 3, 4, 5, 6, or 7);
k is an integer from 1 to 7 (e.g., 1, 2, 3, 4, 5, 6, or 7); and
I is an integer from 1 to 7 (e.g., 1, 2, 3, 4, 5, 6, or 7).

In some embodiments, the antisense strand has a structure represented by Formula A4, wherein Formula A4 is, in the 5′-to-3′ direction:

A-S-B-S-A-O-A-O-A-O-B-O-A-O-A-O-A-O-A-O-A-O-A-O-A-O-B-O-A-O-B-S-A-S-A-S-A-S-B-S-A Formula A4;

wherein A represents a 2′-O-Me ribonucleoside, B represents a 2′-F ribonucleoside, 0 represents a phosphodiester internucleoside linkage, and S represents a phosphorothioate internucleoside linkage.

In some embodiments, the sense strand has a structure represented by Formula S-VII, wherein Formula S-VII is, in the 5′-to-3′ direction:

H-Bₘ-Iₙ-A′-Bₒ-H-C Formula S-VII;
wherein A′ is represented by the formula C-P²-D-P²;
each H is represented by the formula (C-P¹)₂;
each I is represented by the formula (D-P²);
B, C, D, P¹ and P² are as defined in Formula VI;
m is an integer from 1 to 7 (e.g., 1, 2, 3, 4, 5, 6, or 7);
n is an integer from 1 to 7 (e.g., 1, 2, 3, 4, 5, 6, or 7); and
o is an integer from 1 to 7 (e.g., 1, 2, 3, 4, 5, 6, or 7).

In some embodiments, the sense strand has a structure represented by Formula S9, wherein Formula S9 is, in the 5′-to-3′ direction:

A-S-A-S-A-O-A-O-A-O-B-O-B-O-B-O-A-O-B-O-A-O-A-O-A-O-A-S-A-S-A Formula S9;

wherein A represents a 2′-O-Me ribonucleoside, B represents a 2′-F ribonucleoside, 0 represents a phosphodiester internucleoside linkage, and S represents a phosphorothioate internucleoside linkage.

In some embodiments, the antisense strand also has a 5′ phosphorus stabilizing moiety at the 5′ end of the antisense strand.

In some embodiments, the sense strand also has a 5′ phosphorus stabilizing moiety at the 5′ end of the sense strand.

In some embodiments, each 5′-phosphorus stabilizing moiety is, independently, represented by any one of Formula I-VIII:

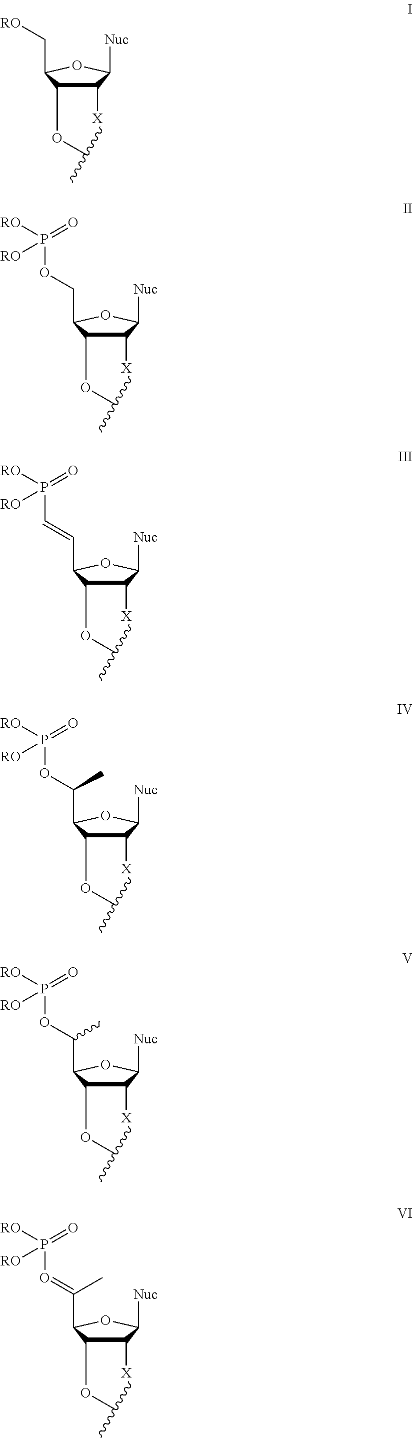

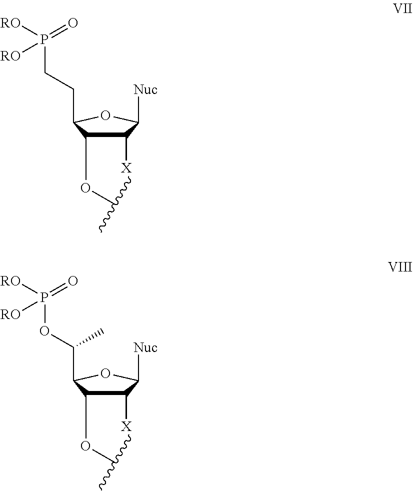

wherein Nuc represents a nucleobase, such as adenine, uracil, guanine, thymine, or cytosine, and R represents optionally substituted alkyl, optionally substituted alkenyl, or optionally substituted alkynyl (e.g., optionally substituted C1-C6 alkyl, optionally substituted C2-C6 alkenyl, or optionally substituted C2-C6 alkynyl), phenyl, benzyl, hydroxy, or hydrogen.

In some embodiments, Z is (E)-vinylphosphonate as represented in Formula III.

In some embodiments, each P is independently selected from phosphodiester and phosphorothioate.

In some embodiments, n is from 1 to 4 (e.g., 1, 2, 3, or 4), 1 to 3 (e.g., 1, 2, or 3), or 1 to 2. In some embodiments, n is 1.

In some embodiments, m is from 1 to 4 (e.g., 1, 2, 3, or 4), 1 to 3 (e.g., 1, 2, or 3), or 1 to 2. In some embodiments, m is 1.

In some embodiments, n and m are each 1.

In some embodiments, 50% or more of the ribonucleotides in the antisense strand are 2′-O-Me ribonucleotides (e.g., 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of the ribonucleotides in the antisense strand may be 2′-O-Me ribonucleotides).

In some embodiments, 60% or more of the ribonucleotides in the antisense strand are 2′-O-Me ribonucleotides (e.g., 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of the ribonucleotides in the antisense strand may be 2′-O-Me ribonucleotides).

In some embodiments, 70% or more of the ribonucleotides in the antisense strand are 2′-O-Me ribonucleotides (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of the ribonucleotides in the antisense strand may be 2′-O-Me ribonucleotides).

In some embodiments, 80% or more of the ribonucleotides in the antisense strand are 2′-O-Me ribonucleotides (e.g., 80%, 81%, 82%, 83%, 84%, 85%, 86%, 87%, 88%, 89%, 90%, 91%, 92%, 93%, 94%, 95%, 96%, 97%, 98%, 99%, or 100% of the ribonucleotides in the antisense strand may be 2′-O-Me ribonucleotides).

In some embodiments, 90% or more of the ribonucleotides in the antisense strand are 2′-O-Me ribonucleotides (e.g., 90%, 91%, 92%, 93%, 94%, 95%, 96%, 97%, 98%, 99%, or 100% of the ribonucleotides in the antisense strand may be 2′-O-Me ribonucleotides).

In some embodiments, 10% or less of the internucleoside linkages are phosphodiester linkages or phosphorothioate. In some embodiments, at least 10%, 20%, 30%, 40%, 50%, 60%, 70%, 80%, or 90% of the internucleoside linkages are phosphodiester linkages or phosphorothioate. In some embodiments, 100% of the internucleoside linkages are phosphodiester linkages or phosphorothioate.

In some embodiments, the length of the antisense strand is between 10 and 30 nucleotides (e.g., nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, or 30 nucleotides), 15 and 25 nucleotides (e.g., 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, or 25 nucleotides), or 18 and 23 nucleotides (e.g., 18 nucleotides, 19 nucleotides, 20 nucleotides, 21 nucleotides, 22 nucleotides, or 23 nucleotides). In some embodiments, the length of the antisense strand is 21 nucleotides. In some embodiments, the length of the antisense strand is 22 nucleotides. In some embodiments, the length of the antisense strand is 23 nucleotides. In some embodiments, the length of the antisense strand is 24 nucleotides. In some embodiments, the length of the antisense strand is 25 nucleotides. In some embodiments, the length of the antisense strand is 26 nucleotides. In some embodiments, the length of the antisense strand is 27 nucleotides. In some embodiments, the length of the antisense strand is 28 nucleotides. In some embodiments, the length of the antisense strand is 29 nucleotides. In some embodiments, the length of the antisense strand is 30 nucleotides.

In some embodiments, 9 internucleoside linkages are phosphorothioate.

In some embodiments, the sense strand of the branched siRNA has the following formula in the 5′-to-3′ direction:

Y-((A-P-)ₙ(B-P-)ₘ)_{q}L-((B-P-)ₘ(A-P-)ₙ)_{q};

wherein Y is a hydrophobic moiety (e.g., cholesterol, vitamin D, or tocopherol); Lisa linker; each A is, independently, a 2′-O-Me ribonucleoside; each B is, independently, a 2′-fluoro-ribonucleoside; each P is, independently, an internucleoside linkage selected from a phosphodiester linkage and a phosphorothioate linkage; n is an integer from 1 to 5 (1, 2, 3, 4, or 5); M is an integer from 1 to 5 (1, 2, 3, 4, or 5); and q is an integer between 1 and 15 (1, 2, 3, 4, 5, 6, 7, 8, 9, 10, 11, 12, 13, 14, or 15).

In some embodiments, Y is cholesterol.

In some embodiments, Y tocopherol.

In some embodiments, L is an ethylene glycol oligomer.

In some embodiments, L is tetraethylene glycol.

In some embodiments, each P is independently selected from phosphodiester and phosphorothioate.

In some embodiments, n is from 1 to 4 (e.g., 1, 2, 3, or 4), 1 to 3 (e.g., 1, 2, or 3), or 1 to 2. In some embodiments, n is 1.

In some embodiments, m is from 1 to 4 (e.g., 1, 2, 3, or 4), 1 to 3 (e.g., 1, 2, or 3), or 1 to 2. In some embodiments, m is 1.

In some embodiments, n and m are each 1.

In some embodiments, 10% or less of the ribonucleosides are 2′-O-Me ribonucleoside.

In some embodiments, at least 10%, 20%, 30%, 40%, 50%, 60%, 70%, 80%, or 90% of the ribonucleosides are 2′-O-Me ribonucleoside.

In some embodiments, 10% or less of the internucleoside linkages are phosphodiester linkages or phosphorothioate linkages. In some embodiments, at least 10%, 20%, 30%, 40%, 50%, 60%, 70%, 80%, or 90% of the internucleoside linkages are phosphodiester linkages or phosphorothioate linkages. In some embodiments, 100% of the internucleoside linkages are phosphodiester linkages or phosphorothioate linkages.

In some embodiments, the length of the sense strand is between 12 and 30 nucleotides (e.g., 12 nucleotides, 13 nucleotides, 14 nucleotides, 15 nucleotides, 16 nucleotides, 17 nucleotides, 18 nucleotides, 19 nucleotides, 20 nucleotides, 21, nucleotides, 22 nucleotides, 23 nucleotides, 24 nucleotides, 25 nucleotides, 26 nucleotides, 27 nucleotides, 28 nucleotides, 29 nucleotides, or 30 nucleotides), or 14 and 18 nucleotides (e.g., 14 nucleotides, 15 nucleotides, 16 nucleotides, 17 nucleotides, 18 nucleotides). In some embodiments, the length of the sense strand is 16 nucleotides. In some embodiments, the length of the sense strand is 17 nucleotides. In some embodiments, the length of the sense strand is 18 nucleotides. In some embodiments, the length of the sense strand is 19 nucleotides. In some embodiments, the length of the sense strand is 20 nucleotides. In some embodiments, the length of the sense strand is 21 nucleotides. In some embodiments, the length of the sense strand is 22 nucleotides. In some embodiments, the length of the sense strand is 23 nucleotides. In some embodiments, the length of the sense strand is 24 nucleotides. In some embodiments, the length of the sense strand is 25 nucleotides. In some embodiments, the length of the sense strand is 26 nucleotides. In some embodiments, the length of the sense strand is 27 nucleotides. In some embodiments, the length of the sense strand is 28 nucleotides. In some embodiments, the length of the sense strand is 29 nucleotides. In some embodiments, the length of the sense strand is 30 nucleotides.

In some embodiments, 4 internucleoside linkages are phosphorothioate.

In another aspect, the invention features a method of treating a subject diagnosed as having a disease associated with expression of a dysregulated microglial gene (e.g., wild-type or mutated microglial gene), the method includes administering to the subject the branched siRNA molecule of any one of the above aspects or embodiments.

In some embodiments, the dysregulated microglial gene is selected from the group consisting of ABCA7, ABI3, ADAM10, APOC1, APOE, AXL, BIN1, C1QA, C3, C9ORF72, CASS4, CCL5, CD2AP, CD33, CD68, CLPTM1, CLU, CR1, CSF1, CST7, CTSB, CTSD, CTSL, CXCL10, CXCL13, DSG2, ECHDC3, EPHA1, FABP5, FERMT2, FTH1, GNAS, GRN, HBEGF, HLA-DRB1, HLA-DRB5, IFIT1, IFIT3, IFITM3, IFNAR1, IFNAR2, IGF1, IL10RA, ILIA, IL1B, IL1RAP, INPP5D, ITGAM, ITGAX, LILRB4, LPL, MEF2C, MMP12, MS4A4A, MS4A6A, NLRP3, NME8, NOS2, PICALM, PILRA, PLCG2, PTK2B, SCIMP, SLC24A4, SORL1, SPI1, SPP1, SPPL2A, TBK1, TNF, TREM2, TREML2, TYROBP, and ZCWPW1.

In some embodiments, the dysregulated microglial gene exhibits increased expression and/or activity in microglial cells of the subject as compared to the expression and/or activity of the same gene in microglial cells of a reference subject.

In some embodiments, the dysregulated microglial gene exhibits reduced expression and/or activity in microglial cells of the subject as compared to the expression and/or activity of the same gene in microglial cells of a reference subject.

In some embodiments, the administering of the branched siRNA molecule to the subject results in silencing of gene in the subject.

In some embodiments, the silencing of a gene comprises silencing any one of the genes selected from the group consisting of APOE, BIN1, C1QA, C3, C9ORF72, CCL5, CD33, CLU/APOJ, CR1, CXCL10, CXCL13, IFIT1, IFIT3, IFITM3, IFNAR1, IFNAR2, IL10RA, ILIA, IL1B, IL1RAP, INPP5D, ITGAM, MEF2C, MMP12, NLRP3, NOS2, PILRA, PLCG2, PTK2B, SLC24A4, TBK1, and TNF.

In some embodiments, silencing of a gene comprises silencing of a positive regulator of a gene for which increased expression and/or activity relative to the level of expression and/or activity observed in a reference subject is associated with a disease state.

In some embodiments, silencing of a gene comprises silencing of a negative regulator of a gene for which decreased expression and/or activity relative to the level of expression and/or activity observed in a reference subject is associated with a disease state.

In some embodiments, silencing of a gene comprises silencing of a splice isoform of a gene for which overexpression of the splice isoform relative to the expression of the splice isoform in a reference subject is associated with a disease state.

In some embodiments, the subject is a human.

### BRIEF DESCRIPTION OF THE DRAWINGS

FIGS. 1A-1D are a series of fluorescence images of brain and spinal cord tissue of cynomolgus macaques treated with a single intrathecal injection of Cy3-labeled di-siRNA of the disclosure. Fluorescence images were acquired from representative regions of the brain, including cortex (FIG. 1A), hippocampus (FIG. 1B), caudate nucleus (FIG. 1C), and of the spinal cord (FIG. 1D). Microglia cells (Iba1 channel), di-siRNAs (Cy3 channel), and cell nuclei (DAPI) were labeled. White arrows indicate colocalization of Cy3 di-siRNA signal within microglial cells labeled with the Iba1 antibody. Scale bars=20 μm.

### DEFINITIONS

Unless otherwise defined herein, scientific, and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

As used herein, the term “nucleic acids” refers to RNA or DNA molecules consisting of a chain of ribonucleotides or deoxyribonucleotides, respectively. As used herein, the term “therapeutic nucleic acid” refers to a nucleic acid molecule (e.g., ribonucleic acid) that has partial or complete complementarity to, and interacts with, a disease-associated target mRNA and mediates silencing of expression of the mRNA.

As used herein, the term “carrier nucleic acid” refers to a nucleic acid molecule (e.g., ribonucleic acid) that has sequence complementarity with, and hybridizes with, a therapeutic nucleic acid. As used herein, the term “3′ end” refers to the end of the nucleic acid that contains an unmodified hydroxyl group at the 3′ carbon of the ribose ring.

As used herein, the term “nucleoside” refers to a molecule made up of a heterocyclic base and its sugar.

As used herein, the term “nucleotide” refers to a nucleoside having a phosphate group on its 3′ or 5′ sugar hydroxyl group.

As used herein, the term “siRNA” refers to small interfering RNA duplexes that induce the RNA interference (RNAi) pathway. siRNA molecules can vary in length (generally, between 18-30 base pairs) and contain varying degrees of complementarity to their target mRNA. The term “siRNA” includes duplexes of two separate strands, as well as single strands that optionally form hairpin structures comprising a duplex region.

As used herein, the term “antisense strand” refers to the strand of the siRNA duplex that contains some degree of complementarity to the target gene.

As used herein, the term “sense strand” refers to the strand of the siRNA duplex that contains complementarity to the antisense strand.

As used herein, the terms “chemically modified nucleotide” or “nucleotide analog” or “altered nucleotide” or “modified nucleotide” refer to a non-standard nucleotide, including non-naturally occurring ribonucleotides or deoxyribonucleotides. Exemplary nucleotide analogs are modified at any position so as to alter certain chemical properties of the nucleotide yet retain the ability of the nucleotide analog to perform its intended function.

As used herein, the term “metabolically stabilized” refers to RNA molecules that contain ribonucleotides that have been chemically modified from 2′-hydroxyl groups to 2′-O-methyl groups.

As used herein, the term “phosphorothioate” refers to the phosphate group of a nucleotide that is modified by substituting one or more of the oxygens of the phosphate group with sulfur.

As used herein, the term “ethylene glycol chain” refers to a carbon chain with the formula ((CH₂OH)₂).

As used herein, “alkyl” refers to a saturated hydrocarbon group. Alkyl groups may be acyclic or cyclic and contain only C and H when unsubstituted. When an alkyl residue having a specific number of carbons is named, all geometric isomers having that number of carbons are intended to be encompassed and described; thus, for example, “butyl” is meant to include n-butyl, sec-butyl, and iso-butyl. Examples of alkyl include ethyl, propyl, butyl, pentyl, hexyl, heptyl, octyl, cyclopropyl, cyclobutyl, cyclopentyl, cyclohexyl, cycloheptyl, cyclooctyl, and the like. In some embodiments, alkyl may be substituted. Suitable substituents that may be introduced into an alkyl group include, for example, hydroxy, alkoxy, amino, alkylamino, and halo, among others.

As used herein, “alkenyl” refers to an acyclic or cyclic unsaturated hydrocarbon group having at least one site of olefinic unsaturation (i.e., having at least one moiety of the formula C═C). Alkenyl groups contain only C and H when unsubstituted. When an alkenyl residue having a specific number of carbons is named, all geometric isomers having that number of carbons are intended to be encompassed and described; thus, for example, “butenyl” is meant to include n-butenyl, sec-butenyl, and iso-butenyl. Examples of alkenyl include —CH═CH₂, —CH₂—CH═CH₂, and —CH₂—CH═CH—CH═CH₂. In some embodiments, alkenyl may be substituted. Suitable substituents that may be introduced into an alkenyl group include, for example, hydroxy, alkoxy, amino, alkylamino, and halo, among others.

As used herein, “alkynyl” refers to an acyclic or cyclic unsaturated hydrocarbon group having at least one site of acetylenic unsaturation (i.e., having at least one moiety of the formula C≡C). Alkynyl groups contain only C and H when unsubstituted. When an alkynyl residue having a specific number of carbons is named, all geometric isomers having that number of carbons are intended to be encompassed and described; thus, for example, “pentynyl” is meant to include n-pentynyl, sec-pentynyl, iso-pentynyl, and ted-pentynyl. Examples of alkynyl include —C≡CH and —C≡C—CH₃. In some embodiments, alkynyl may be substituted. Suitable substituents that may be introduced into an alkynyl group include, for example, hydroxy, alkoxy, amino, alkylamino, and halo, among others.

As used herein the term “phenyl” denotes a monocyclic arene in which one hydrogen atom from a carbon atom of the ring has been removed. A phenyl group can be unsubstituted or substituted with one or more suitable substituents, wherein the substituent replaces an H of the phenyl group.

As used herein, the term “benzyl” refers to monovalent radical obtained when a hydrogen atom attached to the methyl group of toluene is removed. A benzyl generally has the formula of phenyl-CH₂—. A benzyl group can be unsubstituted or substituted with one or more suitable substituents. For example, the substituent may replace an H of the phenyl component and/or an H of the methylene (—CH₂—) component.

As used herein, the term “amide” refers to an alkyl or aromatic group that is attached to an amino-carbonyl functional group.

As used herein, the term “internucleoside” and “internucleotide” refer to the bonds between nucleosides and nucleotides, respectively.

As used herein, the term “triazol” refers to heterocyclic compounds with the formula (C2H₃N₃), having a five-membered ring of two carbons and three nitrogens, the positions of which can change resulting in multiple isomers.

As used herein, the term “terminal group” refers to the group at which a carbon chain or nucleic acid ends.

As used herein, the term “lipophilic amino acid” refers to an amino acid comprising a hydrophobic moiety (e.g., an alkyl chain or an aromatic ring).

As used herein, the term “antagomiRs” refers to nucleic acids that can function as inhibitors of miRNA activity.

As used herein, the term “gapmers” refers to chimeric antisense nucleic acids that contain a central block of deoxynucleotide monomers sufficiently long to induce RNase H cleavage. The deoxynucleotide block is flanked by ribonucleotide monomers or ribonucleotide monomers containing modifications.

As used herein, the term “mixmers” refers to nucleic acids that are comprised of a mix of locked nucleic acids (LNAs) and DNA.

As used herein, the term “guide RNAs” refers to nucleic acids that have sequence complementarity to a specific sequence in the genome immediately or 1 base pair upstream of the protospacer adjacent motif (PAM) sequence as used in CRISPR/Cas9 gene editing systems. Alternatively, “guide RNAs” may refer to nucleic acids that have sequence complementarity (e.g., are antisense) to a specific messenger RNA (mRNA) sequence. In this context, a guide RNA may also have sequence complementarity to a “passenger RNA” sequence of equal or shorter length, which is identical or substantially identical to the sequence of mRNA to which the guide RNA hybridizes.

As used herein, the term “target of delivery” refers to the organ or part of the body that is desired to deliver the branched oligonucleotide compositions to.

As used herein, the term “branched siRNA” refers to a compound containing two or more double-stranded siRNA molecules covalently bound to one another. Branched siRNA molecules may be “di-branched,” also referred to herein as “di-siRNA,” wherein the siRNA molecule comprises 2 siRNA molecules covalently bound to one another, e.g., by way of a linker. Branched siRNA molecules may be “tri-branched,” also referred to herein as “tri-siRNA,” wherein the siRNA molecule comprises 3 siRNA molecules covalently bound to one another, e.g., by way of a linker. Branched siRNA molecules may be “tetra-branched,” also referred to herein as “tetra-siRNA,” wherein the siRNA molecule comprises 4 siRNA molecules covalently bound to one another, e.g., by way of a linker.

As used herein, the term “5′ phosphorus stabilizing moiety” refers to a terminal phosphate group that includes phosphates as well as modified phosphates (e.g., phosphorothioates, phosphodiesters, phosphonates). The phosphate moiety can be located at either terminus but is preferred at the 5′-terminal nucleoside. In one aspect, the terminal phosphate is unmodified having the formula —O—P(═O)(OH)OH. In another aspect, the terminal phosphate is modified such that one or more of the O and OH groups are replaced with H, O, S, N(R′), or alkyl where R′ is H, an amino protecting group, or unsubstituted or substituted alkyl. In some embodiments, the 5′ and or 3′ terminal group can comprise from 1 to 3 phosphate moieties that are each, independently, unmodified (di- or tri-phosphates) or modified.

As used herein, the term “between X and Y” is inclusive of the values of X and Y. For example, “between X and Y” refers to the range of values between the value of X and the value of Y, as well as the value of X and the value of Y.

As used herein, an “amino acid” refers to a molecule containing amine and carboxyl functional groups and a side chain specific to the amino acid:

In some embodiments the amino acid is chosen from the group of proteinogenic amino acids. In other embodiments, the amino acid is an L-amino acid or a D-amino acid. In other embodiments, the amino acid is a synthetic amino acid (e.g., a beta-amino acid).

It is understood that certain internucleotide linkages provided herein, including, e.g., phosphodiester and phosphorothioate, comprise a formal charge of −1 at physiological pH, and that said formal charge will be balanced by a cationic moiety, e.g., an alkali metal such as sodium or potassium, an alkali earth metal such as calcium or magnesium, or an ammonium or guanidinium ion.

The phosphate group of the nucleotide may also be modified, e.g., by substituting one or more of the oxygens of the phosphate group with sulfur (e.g., phosphorothioates), or by making other substitutions which allow the nucleotide to perform its intended function such as described in, for example, Eckstein, Antisense Nucleic Acid Drug Dev. 2000 Apr. 10(2):117-21, Rusckowski et al. Antisense Nucleic Acid Drug Dev. 2000 Oct. 10(5):333-45, Stein, Antisense Nucleic Acid Drug Dev. 2001 Oct. 11(5): 317-25, Vorobjev et al. Antisense Nucleic Acid Drug Dev. 2001 Apr. 11(2):77-85, and U.S. Pat. No. 5,684,143. Certain of the above-referenced modifications (e.g., phosphate group modifications) preferably decrease the rate of hydrolysis of, for example, polynucleotides comprising said analogs in vivo or in vitro.

As used herein, the term “complementary” refers to two nucleotides that form canonical Watson-Crick base pairs. For the avoidance of doubt, Watson-Crick base pairs in the context of the present disclosure include adenine-thymine, adenine-uracil, and cytosine-guanine base pairs. A proper Watson-Crick base pair is referred to in this context as a “match,” while each unpaired nucleotide, and each incorrectly paired nucleotide, is referred to as a “mismatch.” Alignment for purposes of determining percent nucleic acid sequence complementarity can be achieved in various ways that are within the capabilities of one of skill in the art, for example, using publicly available computer software such as BLAST, BLAST-2, or Megalign software.

As used herein, the term “percent (%) sequence complementarity” with respect to a reference polynucleotide sequence is defined as the percentage of nucleic acids in a candidate sequence that are complementary to the nucleic acids in the reference polynucleotide sequence, after aligning the sequences and introducing gaps, if necessary, to achieve the maximum percent sequence complementarity. A given nucleotide is considered to be “complementary” to a reference nucleotide as described herein if the two nucleotides form canonical Watson-Crick base pairs. For the avoidance of doubt, Watson-Crick base pairs in the context of the present disclosure include adenine-thymine, adenine-uracil, and cytosine-guanine base pairs. A proper Watson-Crick base pair is referred to in this context as a “match,” while each unpaired nucleotide, and each incorrectly paired nucleotide, is referred to as a “mismatch.” Alignment for purposes of determining percent nucleic acid sequence complementarity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal complementarity over the full length of the sequences being compared. As an illustration, the percent sequence complementarity of a given nucleic acid sequence, A, to a given nucleic acid sequence, B, (which can alternatively be phrased as a given nucleic acid sequence, A that has a certain percent complementarity to a given nucleic acid sequence, B) is calculated as follows:

100 multiplied by (the fraction X/Y)

where X is the number of complementary base pairs in an alignment (e.g., as executed by computer software, such as BLAST) in that program's alignment of A and B, and where Y is the total number of nucleic acids in B. It will be appreciated that where the length of nucleic acid sequence A is not equal to the length of nucleic acid sequence B, the percent sequence complementarity of A to B will not equal the percent sequence complementarity of B to A. As used herein, a query nucleic acid sequence is considered to be “completely complementary” to a reference nucleic acid sequence if the query nucleic acid sequence has 100% sequence complementarity to the reference nucleic acid sequence.

The term “gene silencing” refers to the suppression of gene expression, e.g., transgene, heterologous gene and/or endogenous gene expression, which may be mediated through processes that affect transcription and/or through processes that affect post-transcriptional mechanisms. In some embodiments, gene silencing occurs when an RNAi molecule initiates the inhibition or degradation of the mRNA transcribed from a gene of interest in a sequence-specific manner via RNA interference, thereby preventing translation of the gene's product.

The phrase “overactive disease driver gene,” as used herein, refers to a microglial gene having increased activity and/or expression that contributes to or causes a disease state in a subject (e.g., a human). The disease state may be caused or exacerbated by the overactive disease driver gene directly or by way of an intermediate gene(s).

The term “negative regulator,” as used herein, refers to a microglial gene that negatively regulates (e.g., reduces or inhibits) the expression and/or activity of another microglial gene or set of genes (e.g., dysregulated microglial gene or dysregulated microglial gene pathway).

The term “positive regulator,” as used herein, refers to a microglial gene that positively regulates (e.g., increases or saturates) the expression and/or activity of another microglial gene or set of microglial genes (e.g., dysregulated microglial gene or dysregulated microglial gene pathway).

The term “phosphate moiety” as used herein, refers to a terminal phosphate group that includes phosphates as well as modified phosphates. The phosphate moiety can be located at either terminus but is preferred at the 5′-terminal nucleoside. In one aspect, the terminal phosphate is unmodified having the formula —O—P(═O)(OH)OH. In another aspect, the terminal phosphate is modified such that one or more of the O and OH groups are replaced with H, O, S, N(R′) or alkyl where R′ is H, an amino protecting group or unsubstituted or substituted alkyl. In some embodiments, the 5′ and or 3′ terminal group can comprise from 1 to 3 phosphate moieties that are each, independently, unmodified (di or tri-phosphates) or modified.

In the context of this invention, the term “oligonucleotide” refers to an oligomer or polymer of ribonucleic acid (RNA) or deoxyribonucleic acid (DNA) or mimetics thereof. This term includes oligonucleotides composed of naturally-occurring nucleobases, sugars and covalent internucleoside (backbone) linkages as well as oligonucleotides having non-naturally-occurring portions that function similarly. Such modified or substituted oligonucleotides are often preferred over native forms because of desirable properties such as, for example, enhanced cellular uptake, enhanced affinity for nucleic acid target and increased stability in the presence of nucleases.

As used herein, the term “reference subject” refers to a healthy control subject of the same or similar, e.g., age, sex, geographical region, and/or education level as a subject treated with a composition of the disclosure. A healthy reference subject is one that does not suffer from a disease associated with expression of a dysregulated microglial gene or a dysregulated microglial gene pathway. Moreover, a healthy reference subject is one that does not suffer from a disease associated with altered (e.g., increased or decreased) expression and/or activity of a microglial gene.

As used herein, the terms “treat,” “treated,” or “treating” mean both therapeutic treatment and prophylactic or preventative measures wherein the object is to prevent or slow down (lessen) an undesired physiological condition, disorder, or disease, or obtain beneficial or desired clinical results. Beneficial or desired clinical results include, but are not limited to, alleviation of symptoms; diminishment of the extent of a condition, disorder, or disease; stabilized (i.e., not worsening) state of condition, disorder, or disease; delay in onset or slowing of condition, disorder, or disease progression; amelioration of the condition, disorder, or disease state or remission (whether partial or total), whether detectable or undetectable; an amelioration of at least one measurable physical parameter, not necessarily discernible by the patient; or enhancement or improvement of condition, disorder, or disease. Treatment includes eliciting a clinically significant response without excessive levels of side effects. Treatment also includes prolonging survival as compared to expected survival if not receiving treatment.

### Genes Described Herein

As used herein, the term “ABCA7” refers to the gene encoding Phospholipid-transporting ATPase ABCA7. The terms “ABCA7” and “Phospholipid-transporting ATPase ABCA7” include wild-type forms of the ABCA7 gene, as well as variants (e.g., splice variants and polymorphisms) of wild-type ABCA7. Examples of such variants are nucleic acids having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to a wild-type ABCA7 nucleic acid sequence (e.g., SEQ ID NO: 1, European Nucleotide Archive (ENA) accession number AF250238). SEQ ID NO: 1 is a wild-type gene sequence encoding ABCA7 protein, and is shown below:

(SEQ ID NO: 1)
ATGGCCTTCTGGACACAGCTGATGCTGCTGCTCTGGAAGAATTTCATGTATCGCCGGAGA
CAGCCGGTCCAGCTCCTGGTCGAATTGCTGTGGCCTCTCTTCCTCTTCTTCATCCTGGTG
GCTGTTCGCCACTCCCACCCGCCCCTGGAGCACCATGAATGCCACTTCCCAAACAAGCCA
CTGCCATCGGCGGGCACCGTGCCCTGGCTCCAGGGTCTCATCTGTAATGTGAACAACACC
TGCTTTCCGCAGCTGACACCGGGCGAGGAGCCCGGGCGCCTGAGCAACTTCAACGACTCC
CTGGTCTCCCGGCTGCTAGCCGATGCCCGCACTGTGCTGGGAGGGGCCAGTGCCCACAGG
ACGCTGGCTGGCCTAGGGAAGCTGATCGCCACGCTGAGGGCTGCACGCAGCACGGCCCAG
CCTCAACCAACCAAGCAGTCTCCACTGGAACCACCCATGCTGGATGTCGCGGAGCTGCTG
ACGTCACTGCTGCGCACGGAATCCCTGGGGTTGGCACTGGGCCAAGCCCAGGAGCCCTTG
CACAGCTTGTTGGAGGCCGCTGAGGACCTGGCCCAGGAGCTCCTGGCGCTGCGCAGCCTG
GTGGAGCTTCGGGCACTGCTGCAGAGACCCCGAGGGACCAGCGGCCCCCTGGAGTTGCTG
TCAGAGGCCCTCTGCAGTGTCAGGGGACCTAGCAGCACAGTGGGCCCCTCCCTCAACTGG
TACGAGGCTAGTGACCTGATGGAGCTGGTGGGGCAGGAGCCAGAATCCGCCCTGCCAGAC
AGCAGCCTGAGCCCCGCCTGCTCGGAGCTGATTGGAGCCCTGGACAGCCACCCGCTGTCC
CGCCTGCTCTGGAGACGCCTGAAGCCTCTGATCCTCGGGAAGCTACTCTTTGCACCAGAT
ACACCTTTTACCCGGAAGCTCATGGCCCAGGTCAACCGGACCTTCGAGGAGCTCACCCTG
CTGAGGGATGTCCGGGAGGTGTGGGAGATGCTGGGACCCCGGATCTTCACCTTCATGAAC
GACAGTTCCAATGTGGCCATGCTGCAGCGGCTCCTGCAGATGCAGGATGAAGGAAGAAGG
CAGCCCAGACCTGGAGGCCGGGACCACATGGAGGCCCTGCGATCCTTTCTGGACCCTGGG
AGCGGTGGCTACAGCTGGCAGGACGCACACGCTGATGTGGGGCACCTGGTGGGCACGCTG
GGCCGAGTGACGGAGTGCCTGTCCTTGGACAAGCTGGAGGCGGCACCCTCAGAGGCAGCC
CTGGTGTCGCGGGCCCTGCAACTGCTCGCGGAACATCGATTCTGGGCCGGCGTCGTCTTC
TTGGGACCTGAGGACTCTTCAGACCCCACAGAGCACCCAACCCCAGACCTGGGCCCCGGC
CACGTGCGCATCAAAATCCGCATGGACATTGACGTGGTCACGAGGACCAATAAGATCAGG
GACAGGTTTTGGGACCCTGGCCCAGCCGCGGACCCCCTGACCGACCTGCGCTACGTGTGG
GGCGGCTTCGTGTACCTGCAAGACCTGGTGGAGCGTGCAGCCGTCCGCGTGCTCAGCGGC
GCCAACCCCCGGGCCGGCCTCTACCTGCAGCAGATGCCCTATCCGTGCTATGTGGACGAC
GTGTTCCTGCGTGTGCTGAGCCGGTCGCTGCCGCTCTTCCTGACGCTGGCCTGGATCTAC
TCCGTGACACTGACAGTGAAGGCCGTGGTGCGGGAGAAGGAGACGCGGCTGCGGGACACC
ATGCGCGCCATGGGGCTCAGCCGCGCGGTGCTCTGGCTAGGCTGGTTCCTCAGCTGCCTC
GGGCCCTTCCTGCTCAGCGCCGCACTGCTGGTTCTGGTGCTCAAGCTGGGAGACATCCTC
CCCTACAGCCACCCGGGCGTGGTCTTCCTGTTCTTGGCAGCCTTCGCGGTGGCCACGGTG
ACCCAGAGCTTCCTGCTCAGCGCCTTCTTCTCCCGCGCCAACCTGGCTGCGGCCTGCGGC
GGCCTGGCCTACTTCTCCCTCTACCTGCCCTACGTGCTGTGTGTGGCTTGGCGGGACCGG
CTGCCCGCGGGTGGCCGCGTGGCCGCGAGCCTGCTGTCGCCCGTGGCCTTCGGCTTCGGC
TGCGAGAGCCTGGCTCTGCTGGAGGAGCAGGGCGAGGGCGCGCAGTGGCACAACGTGGGC
ACCCGGCCTACGGCAGACGTCTTCAGCCTGGCCCAGGTCTCTGGCCTTCTGCTGCTGGAC
GCGGCGCTCTACGGCCTCGCCACCTGGTACCTGGAAGCTGTGTGCCCAGGCCAGTACGGG
ATCCCTGAACCATGGAATTTTCCTTTTCGGAGGAGCTACTGGTGCGGACCTCGGCCCCCC
AAGAGTCCAGCCCCTTGCCCCACCCCGCTGGACCCAAAGGTGCTGGTAGAAGAGGCACCG
CCCGGCCTGAGTCCTGGCGTCTCCGTTCGCAGCCTGGAGAAGCGCTTTCCTGGAAGCCCG
CAGCCAGCCCTGCGGGGGCTCAGCCTGGACTTCTACCAGGGCCACATCACCGCCTTCCTG
GGCCACAACGGGGCCGGCAAGACCACCACCCTGTCCATCTTGAGTGGCCTCTTCCCACCC
AGTGGTGGCTCTGCCTTCATCCTGGGCCACGACGTCCGCTCCAGCATGGCCGCCATCCGG
CCCCACCTGGGCGTCTGTCCTCAGTACAACGTGCTGTTTGACATGCTGACCGTGGACGAG
CACGTCTGGTTCTATGGGCGGCTGAAGGGTCTGAGTGCCGCTGTAGTGGGCCCCGAGCAG
GACCGTCTGCTGCAGGATGTGGGGCTGGTCTCCAAGCAGAGTGTGCAGACTCGCCACCTC
TCTGGTGGGATGCAACGGAAGCTGTCCGTGGCCATTGCCTTTGTGGGCGGCTCCCAAGTT
GTTATCCTGGACGAGCCTACGGCTGGCGTGGATCCTGCTTCCCGCCGCGGTATTTGGGAG
CTGCTGCTCAAATACCGAGAAGGTCGCACGCTGATCCTCTCCACCCACCACCTGGATGAG
GCAGAGCTGCTGGGAGACCGTGTGGCTGTGGTGGCAGGTGGCCGCTTGTGCTGCTGTGGC
TCCCCACTCTTCCTGCGCCGTCACCTGGGCTCCGGCTACTACCTGACGCTGGTGAAGGCC
CGCCTGCCCCTGACCACCAATGAGAAGGCTGACACTGACATGGAGGGCAGTGTGGACACC
AGGCAGGAAAAGAAGAATGGCAGCCAGGGCAGCAGAGTCGGCACTCCTCAGCTGCTGGCC
CTGGTACAGCACTGGGTGCCCGGGGCACGGCTGGTGGAGGAGCTGCCACACGAGCTGGTG
CTGGTGCTGCCCTACACGGGTGCCCATGACGGCAGCTTCGCCACACTCTTCCGAGAGCTA
GACACGCGGCTGGCGGAGCTGAGGCTCACTGGCTACGGGATCTCCGACACCAGCCTCGAG
GAGATCTTCCTGAAGGTGGTGGAGGAGTGTGCTGCGGACACAGATATGGAGGATGGCAGC
TGCGGGCAGCACCTATGCACAGGCATTGCTGGCCTAGACGTAACCCTGCGGCTCAAGATG
CCGCCACAGGAGACAGCGCTGGAGAACGGGGAACCAGCTGGGTCAGCCCCAGAGACTGAC
CAGGGCTCTGGGCCAGACGCCGTGGGCCGGGTACAGGGCTGGGCACTGACCCGCCAGCAG
CTCCAGGCCCTGCTTCTCAAGCGCTTTCTGCTTGCCCGCCGCAGCCGCCGCGGCCTGTTC
GCCCAGATCGTGCTGCCTGCCCTCTTTGTGGGCCTGGCCCTCGTGTTCAGCCTCATCGTG
CCTCCTTTCGGGCACTACCCGGCTCTGCGGCTCAGTCCCACCATGTACGGTGCTCAGGTG
TCCTTCTTCAGTGAGGACGCCCCAGGGGACCCTGGACGTGCCCGGCTGCTCGAGGCGCTG
CTGCAGGAGGCAGGACTGGAGGAGCCCCCAGTGCAGCATAGCTCCCACAGGTTCTCGGCA
CCAGAAGTTCCTGCTGAAGTGGCCAAGGTCTTGGCCAGTGGCAACTGGACCCCAGAGTCT
CCATCCCCAGCCTGCCAGTGTAGCCAGCCCGGTGCCCGGCGCCTGCTGCCCGACTGCCCG
GCTGCAGCTGGTGGTCCCCCTCCGCCCCAGGCAGTGACCGGCTCTGGGGAAGTGGTTCAG
AACCTGACAGGCCGGAACCTGTCTGACTTCCTGGTCAAGACCTACCCGCGCCTGGTGCGC
CAGGGCCTGAAGACTAAGAAGTGGGTGAATGAGGTCAGGTACGGAGGCTTCTCGCTGGGG
GGCCGAGACCCAGGCCTGCCCTCGGGCCAAGAGTTGGGCCGCTCAGTGGAGGAGTTGTGG
GCGCTGCTGAGTCCCCTGCCTGGCGGGGCCCTCGACCGTGTCCTGAAAAACCTCACAGCC
TGGGCTCACAGCCTGGACGCTCAGGACAGTCTCAAGATCTGGTTCAACAACAAAGGCTGG
CACTCCATGGTGGCCTTTGTCAACCGAGCCAGCAACGCAATCCTCCGTGCTCACCTGCCC
CCAGGCCGGGCCCGCCACGCCCACAGCATCACCACACTCAACCACCCCTTGAACCTCACC
AAGGAGCAGCTGTTTGAGGCTGCATTGATGGCCTCCTCGGTGGACGTCCTCGTCTCCATC
TGTGTGGTCTTTGCCATGTCCTTTGTCCCGGCCAGCTTCACTCTTGTCCTCATTGAGGAG
CGAGTCACCCGAGCCAAGCACCTGCAGCTCATGGGGGGCCTGTCCCCCACCCTCTACTGG
CTTGGCAACTTTCTCTGGGACATGTGTAACTACTTGGTGCCAGCATGCATCGTGGTGCTC
ATCTTTCTGGCCTTCCAGCAGAGGGCATATGTGGCCCCTGCCAACCTGCCTGCTCTCCTG
CTGTTGCTACTACTGTATGGCTGGTCGATCACACCGCTCATGTACCCAGCCTCCTTCTTC
TTCTCCGTGCCCAGCACAGCCTATGTGGTGCTCACCTGCATAAACCTCTTTATTGGCATC
AATGGAAGCATGGCCACCTTTGTGCTTGAGCTCTTCTCTGATCAGAAGCTGCAGGAGGTG
AGCCGGATCTTGAAACAGGTCTTCCTTATCTTCCCCCACTTCTGCTTGGGCCGGGGGCTT
ATTGACATGGTGCGGAACCAGGCCATGGCTGATGCCTTTGAGCGCTTGGGAGACAGGCAG
TTCCAGTCACCCCTGCGCTGGGAGGTGGTCGGCAAGAACCTCTTGGCCATGGTGATACAG
GGGCCCCTCTTCCTTCTCTTCACACTACTGCTGCAGCACCGAAGCCAACTCCTGCCACAG
CCCAGGGTGAGGTCTCTGCCACTCCTGGGAGAGGAGGACGAGGATGTAGCCCGTGAACGG
GAGCGGGTGGTCCAAGGAGCCACCCAGGGGGATGTGTTGGTGCTGAGGAACTTGACCAAG
GTATACCGTGGGCAGAGGATGCCAGCTGTTGACCGCTTGTGCCTGGGGATTCCCCCTGGT
GAGTGTTTTGGGCTGCTGGGTGTGAATGGAGCAGGGAAGACGTCCACGTTTCGCATGGTG
ACGGGGGACACATTGGCCAGCAGGGGCGAGGCTGTGCTGGCAGGCCACAGCGTGGCCCGG
GAACCCAGTGCTGCGCACCTCAGCATGGGATACTGCCCTCAATCCGATGCCATCTTTGAG
CTGCTGACGGGCCGCGAGCACCTGGAGCTGCTTGCGCGCCTGCGCGGTGTCCCGGAGGCC
CAGGTTGCCCAGACCGCTGGCTCGGGCCTGGCGCGTCTGGGACTCTCATGGTACGCAGAC
CGGCCTGCAGGCACCTACAGCGGAGGGAACAAACGCAAGCTGGCGACGGCCCTGGCGCTG
GTTGGGGACCCAGCCGTGGTGTTTCTGGACGAGCCGACCACAGGCATGGACCCCAGCGCG
CGGCGCTTCCTTTGGAACAGCCTTTTGGCCGTGGTGCGGGAGGGCCGTTCAGTGATGCTC
ACCTCCCATAGCATGGAGGAGTGTGAAGCGCTCTGCTCGCGCCTAGCCATCATGGTGAAT
GGGCGGTTCCGCTGCCTGGGCAGCCCGCAACATCTCAAGGGCAGATTCGCGGCGGGTCAC
ACACTGACCCTGCGGGTGCCCGCCGCAAGGTCCCAGCCGGCAGCGGCCTTCGTGGCGGCC
GAGTTCCCTGGGTCGGAGCTGCGCGAGGCACATGGAGGCCGCCTGCGCTTCCAGCTGCCG
CCGGGAGGGCGCTGCGCCCTGGCGCGCGTCTTTGGAGAGCTGGCGGTGCACGGCGCAGAG
CACGGCGTGGAGGACTTTTCCGTGAGCCAGACGATGCTGGAGGAGGTATTCTTGTACTTC
TCCAAGGACCAGGGGAAGGACGAGGACACCGAAGAGCAGAAGGAGGCAGGAGTGGGAGTG
GACCCCGCGCCAGGCCTGCAGCACCCCAAACGCGTCAGCCAGTTCCTCGATGACCCTAGC
ACTGCCGAGACTGTGCTCTGAGCCTCCCTCCCCTGCGGGGCCGCGGGGAGGCCCTGGGAA
TGGCAAGGGCAAGGTAGAGTGCCTAGGAGCCCTGGACTCAGGCTGGCAGAGGGGCTGGTG
CCCTGGAGAAAATAAAGAGAAGGCTGGAGAGAAGCCGTGCTTGGTGAA

As used herein, the term “ABI3” refers to the gene encoding ABI gene family member 3. The terms “ABI3” and “ABI gene family member 3” include wild-type forms of the ABI3 gene, as well as variants (e.g., splice variants and polymorphisms) of wild-type ABI3. Examples of such variants are nucleic acids having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to a wild-type ABI3 nucleic acid sequence (e.g., SEQ ID NO: 2, ENA accession number AF037886). SEQ ID NO: 2 is a wild-type gene sequence encoding ABI3 protein, and is shown below:

(SEQ ID NO: 2)
TCCTATCCACCCTCCACTCCCCTGTCCCTTGGTGACTCATCCCTGAGCTTCCCAAGGAAG
CCCCCACCCTCTGCCCTTTCCTCCCGCCTTCCATGAGTGGAAAATCCACCTCCGCCCCCT
ATAGCAGGCCAGCCCCCTTCCTCCCCAGTCTCCGACCCCATCCCCCAGCCGACCAGTTTC
CTCTCCAGGACCAGGGAGCAATCACAGCTGCCCCGACCTTGGCTTCCTCTGCTGGGTGGG
ATTGGGGGCTGGGCCCCCAAATGGGCCCCTGGCTTCCCCCTTCCTCTGGGCAGGGGACAG
AGAGACACAGGCTCGGGGAGCAGGACTGACTTCCTCTTGTCCCGGAATGAGCATGCCTGC
CCTTTGCAAGCAGGTTTGGGTCTCACGCAGAGGAAACCAAAAGCAATAAGAGGGAGGGAA
GGCAGAGCAACCAATCAAGGGCAGGGTGAGACTCAAAACGAGCGGGCTCCCTGGGGAGCC
AGACAGAGGCTGGGGGTGATGGCGGAGCTACAGCAGCTGCAGGAGTTTGAGATCCCCACT
GGCCGGGAGGCTCTGAGGGGCAACCACAGTGCCCTGCTGCGGGTCGCTGACTACTGCGAG
GACAACTATGTGCAGGCCACAGACAAGCGGAAGGCGCTGGAGGAGACCATGGCCTTCACT
ACCCAGGCACTGGCCAGCGTGGCCTACCAGGTGGGCAACCTGGCCGGGCACACTCTGCGC
ATGTTGGACCTGCAGGGGGCCGCCCTGCGGCAGGTGGAAGCCCGTGTAAGCACGCTGGGC
CAGATGGTGAACATGCATATGGAGAAGGTGGCCCGAAGGGAGATCGGCACCTTAGCCACT
GTCCAGCGGCTGCCCCCCGGCCAGAAGGTCATCGCCCCAGAGAACCTACCCCCTCTCACG
CCCTACTGCAGGAGACCCCTCAACTTTGGCTGCCTGGACGACATTGGCCATGGGATCAAG
GACCTCAGCACGCAGCTGTCAAGAACAGGCACCCTGTCTCGAAAGAGCATCAAGGCCCCT
GCCACACCCGCCTCCGCCACCTTGGGGAGACCACCCCGGATTCCCGAGCCAGTGCACCTG
CCGGTGGTGCCCGACGGCAGACTCTCCGCCGCCTCCTCTGCGTCTTCCCTGGCCTCGGCC
GGCAGCGCCGAAGGTGTCGGTGGGGCCCCCACGCCCAAGGGGCAGGCAGCACCTCCAGCC
CCACCTCTCCCCAGCTCCTTGGACCCACCTCCTCCACCAGCAGCCGTCGAGGTGTTCCAG
CGGCCTCCCACGCTGGAGGAGTTGTCCCCACCCCCACCGGACGAAGAGCTGCCCCTGCCA
CTGGACCTGCCTCCTCCTCCACCCCTGGATGGAGATGAATTGGGGCTGCCTCCACCCCCA
CCAGGATTTGGGCCTGATGAGCCCAGCTGGGTGCCTGCCTCATACTTGGAGAAAGTGGTG
ACACTGTACCCATACACCAGCCAGAAGGACAATGAGCTCTCCTTCTCTGAGGGCACTGTC
ATCTGTGTCACTCGCCGCTACTCCGATGGCTGGTGCGAGGGCGTCAGCTCAGAGGGGACT
GGATTCTTCCCTGGGAACTATGTGGAGCCCAGCTGCTGACAGCCCAGGGCTCTCTGGGCA
GCTGATGTCTGCACTGAGTGGGTTTCATGAGCCCCAAGCCAAAACCAGCTCCAGTCACAG
CTGGACTGGGTCTGCCCACCTCTTGGGCTGTGAGCTGTGTTCTGTCCTTCCTCCCATCGG
AGGGAGAAGGGGTCCTGGGGAGAGAGAATTTATCCAGAGGCCTGCTGCAGATGGGGAAGA
GCTGGAAACCAAGAAGTTTGTCAACAGAGGACCCCTACTCCATGCAGGACAGGGTCTCCT
GCTGCAAGTCCCAACTTTGAATAAAACAGATGATGTCCTGTGACTGCCCCACAGAGATAA
GGGGCCAGGAGGGATTGAAAGGCATCCCAGTTCTAAGGCTGCTGCTAATTACAGCCCCCA
ACCTCCAACCCACCAGCTGACCTAGAAGCAGCATCTTCCCATTTCCTCAGTACCCACAAA
GTGCAGCCCACATTGGACCCCAGACACCCCTCTGCAGCCATTGACTGCAACTTGTTCTTT
TGCCCATTAAAAAAAAAAAAAAAAAAAAA

As used herein, the term “ADAM10” refers to the gene encoding ADAM Metallopeptidase Domain 10. The terms “ADAM10” and “ADAM Metallopeptidase Domain 10” include wild-type forms of the ADAM10 gene, as well as variants (e.g., splice variants and polymorphisms) of wild-type ADAM10. Examples of such variants are nucleic acids having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to a wild-type ADAM10 nucleic acid sequence (e.g., SEQ ID NO: 3, NCBI Reference Sequence: NM_001110.3). SEQ ID NO: 3 is a wild-type gene sequence encoding ADAM10 protein, and is shown below:

(SEQ ID NO: 3)
GCGGCGGCAGGCCTAGCAGCACGGGAACCGTCCCCCGCGCGCATGCGCGCGCCCCTGAAGCGCC
TGGGGGACGGGTAGGGGGGGGAGGTAGGGGCGCGGCTCCGCGTGCCAGTTGGGTGCCCGCGCG
TCACGTGGTGAGGAAGGAGGCGGAGGTCTGAGTTTCGAAGGAGGGGGGGAGAGAAGAGGGAACG
AGCAAGGGAAGGAAAGCGGGGAAAGGAGGAAGGAAACGAACGAGGGGGAGGGAGGTCCCTGTTTT
GGAGGAGCTAGGAGCGTTGCCGGCCCCTGAAGTGGAGCGAGAGGGAGGTGCTTCGCCGTTTCTCC
TGCCAGGGGAGGTCCCGGCTTCCCGTGGAGGCTCCGGACCAAGCCCCTTCAGCTTCTCCCTCCGG
ATCGATGTGCTGCTGTTAACCCGTGAGGAGGCGGCGGCGGCGGCAGCGGCAGCGGAAGATGGTGT
TGCTGAGAGTGTTAATTCTGCTCCTCTCCTGGGCGGGGGGATGGGAGGTCAGTATGGGAATCCTT
TAAATAAATATATCAGACATTATGAAGGATTATCTTACAATGTGGATTCATTACACCAAAAACACCAGC
GTGCCAAAAGAGCAGTCTCACATGAAGACCAATTTTTACGTCTAGATTTCCATGCCCATGGAAGACAT
TTCAACCTACGAATGAAGAGGGACACTTCCCTTTTCAGTGATGAATTTAAAGTAGAAACATCAAATAA
AGTACTTGATTATGATACCTCTCATATTTACACTGGACATATTTATGGTGAAGAAGGAAGTTTTAGCCA
TGGGTCTGTTATTGATGGAAGATTTGAAGGATTCATCCAGACTCGTGGTGGCACATTTTATGTTGAGC
CAGCAGAGAGATATATTAAAGACCGAACTCTGCCATTTCACTCTGTCATTTATCATGAAGATGATATTA
ACTATCCCCATAAATACGGTCCTCAGGGGGGCTGTGCAGATCATTCAGTATTTGAAAGAATGAGGAA
ATACCAGATGACTGGTGTAGAGGAAGTAACACAGATACCTCAAGAAGAACATGCTGCTAATGGTCCA
GAACTTCTGAGGAAAAAACGTACAACTTCAGCTGAAAAAAATACTTGTCAGCTTTATATTCAGACTGA
TCATTTGTTCTTTAAATATTACGGAACACGAGAAGCTGTGATTGCCCAGATATCCAGTCATGTTAAAG
CGATTGATACAATTTACCAGACCACAGACTTCTCCGGAATCCGTAACATCAGTTTCATGGTGAAACGC
ATAAGAATCAATACAACTGCTGATGAGAAGGACCCTACAAATCCTTTCCGTTTCCCAAATATTGGTGT
GGAGAAGTTTCTGGAATTGAATTCTGAGCAGAATCATGATGACTACTGTTTGGCCTATGTCTTCACAG
ACCGAGATTTTGATGATGGCGTACTTGGTCTGGCTTGGGTTGGAGCACCTTCAGGAAGCTCTGGAG
GAATATGTGAAAAAAGTAAACTCTATTCAGATGGTAAGAAGAAGTCCTTAAACACTGGAATTATTACT
GTTCAGAACTATGGGTCTCATGTACCTCCCAAAGTCTCTCACATTACTTTTGCTCACGAAGTTGGACA
TAACTTTGGATCCCCACATGATTCTGGAACAGAGTGCACACCAGGAGAATCTAAGAATTTGGGTCAA
AAAGAAAATGGCAATTACATCATGTATGCAAGAGCAACATCTGGGGACAAACTTAACAACAATAAATT
CTCACTCTGTAGTATTAGAAATATAAGCCAAGTTCTTGAGAAGAAGAGAAACAACTGTTTTGTTGAAT
CTGGCCAACCTATTTGTGGAAATGGAATGGTAGAACAAGGTGAAGAATGTGATTGTGGCTATAGTGA
CCAGTGTAAAGATGAATGCTGCTTCGATGCAAATCAACCAGAGGGAAGAAAATGCAAACTGAAACCT
GGGAAACAGTGCAGTCCAAGTCAAGGTCCTTGTTGTACAGCACAGTGTGCATTCAAGTCAAAGTCTG
AGAAGTGTCGGGATGATTCAGACTGTGCAAGGGAAGGAATATGTAATGGCTTCACAGCTCTCTGCCC
AGCATCTGACCCTAAACCAAACTTCACAGACTGTAATAGGCATACACAAGTGTGCATTAATGGGCAAT
GTGCAGGTTCTATCTGTGAGAAATATGGCTTAGAGGAGTGTACGTGTGCCAGTTCTGATGGCAAAGA
TGATAAAGAATTATGCCATGTATGCTGTATGAAGAAAATGGACCCATCAACTTGTGCCAGTACAGGGT
CTGTGCAGTGGAGTAGGCACTTCAGTGGTCGAACCATCACCCTGCAACCTGGATCCCCTTGCAACG
ATTTTAGAGGTTACTGTGATGTTTTCATGCGGTGCAGATTAGTAGATGCTGATGGTCCTCTAGCTAGG
CTTAAAAAAGCAATTTTTAGTCCAGAGCTCTATGAAAACATTGCTGAATGGATTGTGGCTCATTGGTG
GGCAGTATTACTTATGGGAATTGCTCTGATCATGCTAATGGCTGGATTTATTAAGATATGCAGTGTTC
ATACTCCAAGTAGTAATCCAAAGTTGCCTCCTCCTAAACCACTTCCAGGCACTTTAAAGAGGAGGAG
ACCTCCACAGCCCATTCAGCAACCCCAGCGTCAGCGGCCCCGAGAGAGTTATCAAATGGGACACAT
GAGACGCTAACTGCAGCTTTTGCCTTGGTTCTTCCTAGTGCCTACAATGGGAAAACTTCACTCCAAA
GAGAAACCTATTAAGTCATCATCTCCAAACTAAACCCTCACAAGTAACAGTTGAAGAAAAAATGGCAA
GAGATCATATCCTCAGACCAGGTGGAATTACTTAAATTTTAAAGCCTGAAAATTCCAATTTGGGGGTG
GGAGGTGGAAAAGGAACCCAATTTTCTTATGAACAGATATTTTTAACTTAATGGCACAAAGTCTTAGA
ATATTATTATGTGCCCCGTGTTCCCTGTTCTTCGTTGCTGCATTTTCTTCACTTGCAGGCAAACTTGG
CTCTCAATAAACTTTTACCACAAATTGAAATAAATATATTTTTTTCAACTGCCAATCAAGGCTAGGAGG
CTCGACCACCTCAACATTGGAGACATCACTTGCCAATGTACATACCTTGTTATATGCAGACATGTATT
TCTTACGTACACTGTACTTCTGTGTGCAATTGTAAACAGAAATTGCAATATGGATGTTTCTTTGTATTA
TAAAATTTTTCCGCTCTTAATTAAAAATTACTGTTTAATTGACATACTCAGGATAACAGAGAATGGTGG
TATTCAGTGGTCCAGGATTCTGTAATGCTTTACACAGGCAGTTTTGAAATGAAAATCAATTTACCTTTC
TGTTACGATGGAGTTGGTTTTGATACTCATTTTTTCTTTATCACATGGCTGCTACGGGCACAAGTGAC
TATACTGAAGAACACAGTTAAGTGTTGTGCAAACTGGACATAGCAGCACATACTACTTCAGAGTTCAT
GATGTAGATGTCTGGTTTCTGCTTACGTCTTTTAAACTTTCTAATTCAATTCCATTTTTCAATTAATAGG
TGAAATTTTATTCATGCTTTGATAGAAATTATGTCAATGAAATGATTCTTTTTATTTGTAGCCTACTTAT
TTGTGTTTTTCATATATCTGAAATATGCTAATTATGTTTTCTGTCTGATATGGAAAAGAAAAGCTGTGT
CTTTATCAAAATATTTAAACGGTTTTTTCAGCATATCATCACTGATCATTGGTAACCACTAAAGATGAG
TAATTTGCTTAAGTAGTAGTTAAAATTGTAGATAGGCCTTCTGACATTTTTTTTCCTAAAATTTTTAACA
GCATTGAAGGTGAAACAGCACAATGTCCCATTCCAAATTTATTTTTGAAACAGATGTAAATAATTGGC
ATTTTAAAGAGAAAGCAAAAACATTTAATGTATTAACAGGCTTATTGCTATGCAGGAAATAGAAGGGG
CATTACAAAAATTGAAGCTTGTGACATATTTATTGCTTCTGTTTTCCAACTACATCACTTCAACTAGAA
GTAAAGCTATGATTTTCCTGACTTCACATAGGAGGCAAATTTAGAGAAAGTTGTAAAGATTTCTATGTT
TTGGGTTTTTTTTTTTCCTTTTTTTTTTTAAGAGTATAAGGTTTACACAATCATTCTCATAATGTGACGC
AAGCCAGCAAGGCCAAAAATGCTAGAGAAAATAACGGGATCTCTTCCTTGTAAACTTGTACAGTATGT
GGTGACTTTTTCAAAATACAGCTTTTTGTACATGATTTAGAGACAAATTTTGTACATGAAACCCCAGAT
AGACTATAAATAATTCTAAACAAACAAGTAGGTAGATATGTATGTAATTGCTTTTAAATCATTTAAATGC
CTTTGTTTTTGGACTGTGCAAAGGTTGGAAGTGGGTTTGCATTTCTAAAATGGTGACTTTTATTCTGC
AAGAGTTCTTAGTAACTTCTTGAGTGTGGTAGACTTTGGAACATGTAAATTTTTTGCTTGTAATGTTAT
CCTGTGGTAGGATTTTGGCAGGTACACACACTGCCCTATTTTATTTTGAGTCTAAGTTAAATGTTTTCT
GAAAAGAGATACATGCACTGAACTCTTTCCACTGCGAATCAAGATGTGGTAATATAAAAGGATCAAGA
CAAATGAGATCTAATACTACTGTCAGTTTTAATGTCCACTGTGTTTTATACAGTATCTTTTTTTGTTCAC
TTTGGAAATTTTTACTAAAAATTGCAAAAAATAAAGTATTGTGCAAAGATGTAAGGTTTTTTGAAACTTG
AAATGCATTAATAAATAGACGATTAAATCAACTTGAAGGTTCTATACTCTTTGAACTCTGAGAACTATC
ACAAGAAGCTTCCCACAAGGCAGTGTTTTCTTACAGTTGTCTCTTCCTACAAAAGTATAGATTATCTTT
ATTCTTAATACTTTGGAATCCATGTAGAAAATTTCCAGTTAGATACTCTGCGTACACACAATAAACCTT
TTTAAAACACCCAAAAAAAAAAAAAAAAAA

The terms “APOC1” and “Apolipoprotein C1” include wild-type forms of the APOC1 gene, as well as variants (e.g., splice variants and polymorphisms) of wild-type APOC1. Examples of such variants are nucleic acids having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to a wild-type APOC1 nucleic acid sequence (e.g., SEQ ID NO: 4, NCBI Reference Sequence: NM_001645). SEQ ID NO: 4 is a wild-type gene sequence encoding APOC1 protein, and is shown below:

AACGCTCACGGGACAGGGGCAGAGGAGAAAAACGTGGGTGGACAGAGGGAGGCAGGCGGTCAGG
GGAAGGCTCAGGAGGAGGGAGATCAACATCAACCTGCCCCGCCCCCTCCCCAGCCTGATAAAGGT
CCTGCGGGCAGGACAGGACCTCCCAACCAAGCCCTCCAGCAAGGATTCAGAGTGCCCCTCCGGCC
TCGCCATGAGGCTCTTCCTGTCGCTCCCGGTCCTGGTGGTGGTTCTGTCGATCGTCTTGGAAGGCC
CAGCCCCAGCCCAGGGGACCCCAGACGTCTCCAGTGCCTTGGATAAGCTGAAGGAGTTTGGAAACA
CACTGGAGGACAAGGCTCGGGAACTCATCAGCCGCATCAAACAGAGTGAACTTTCTGCCAAGATGC
GGGAGTGGTTTTCAGAGACATTTCAGAAAGTGAAGGAGAAACTCAAGATTGACTCATGAGGACCTGA
AGGGTGACATCCCAGGAGGGGCCTCTGAAATTTCCCACACCCCAGCGCCTGTGCTGAGGACTCCCT
CCATGTGGCCCCAGGTGCCACCAATAAAAATCCTACAGAAAATTCAAAAAAAAAAAAAAAAAA

### (SEQ ID NO: 4)

As used herein, the term “APOE” refers to the gene encoding Apolipoprotein E. The terms “APOE” and “Apolipoprotein E” include wild-type forms of the APOE gene, as well as variants (e.g., splice variants and polymorphisms) of wild-type APOE. Examples of such variants are nucleic acids having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to a wild-type APOE nucleic acid sequence (e.g., SEQ ID NO: 5, ENA accession number M12529). SEQ ID NO: 5 is a wild-type gene sequence encoding APOE protein, and is shown below:

(SEQ ID NO: 5)
CCCCAGCGGAGGTGAAGGACGTCCTTCCCCAGGAGCCGACTGGCCAATCACAGGCAGGAA
GATGAAGGTTCTGTGGGCTGCGTTGCTGGTCACATTCCTGGCAGGATGCCAGGCCAAGGT
GGAGCAAGCGGTGGAGACAGAGCCGGAGCCCGAGCTGCGCCAGCAGACCGAGTGGCAGAG
CGGCCAGCGCTGGGAACTGGCACTGGGTCGCTTTTGGGATTACCTGCGCTGGGTGCAGAC
ACTGTCTGAGCAGGTGCAGGAGGAGCTGCTCAGCTCCCAAGTCACCCAAGAACTGAGGGC
GCTGATGGACGAGACCATGAAGGAGTTGAAGGCCTACAAATCGGAACTGGAGGAACAACT
GACCCCGGTAGCGGAGGAGACGCGGGCACGGCTGTCCAAGGAGCTGCAGACGGCGCAGGC
CCGGCTGGGCGCGGACATGGAGGACGTGTGCGGCCGCCTGGTGCAGTACCGCGGCGAGGT
GCAGGCCATGCTCGGCCAGAGCACCGAGGAGCTGCGGGTGCGCCTCGCCTCCCACCTGCG
CAAGCTGCGTAAGCGGCTCCTCCGCGATCCCGATGACCTGCAGAAGCGCCTGGCAGTGTA
CCAGGCCGGGGCCCGCGAGGGCGCCGAGCGCGGCCTCAGCGCCATCCGCGAGCGCCTGGG
GCCCCTGGTGGAACAGGGCCGCGTGCGGGCCGCCACTGTGGGCTCCCTGGCCGGCCAGCC
GCTACAGGAGCGGGCCCAGGCCTGGGGCGAGCGGCTGCGCGCGCGGATGGAGGAGATGGG
CAGTCGGACCCGCGACCGCCTGGACGAGGTGAAGGAGCAGGTGGCGGAGGTGCGCGCCAA
GCTGGAGGAGCAGGCCCAGCAGATACGCCTGCAGGCCGAGGCCTTCCAGGCCCGCCTCAA
GAGCTGGTTCGAGCCCCTGGTGGAAGACATGCAGCGCCAGTGGGCCGGGCTGGTGGAGAA
GGTGCAGGCTGCCGTGGGCACCAGCGCCGCCCCTGTGCCCAGCGACAATCACTGAACGCC
GAAGCCTGCAGCCATGCGACCCCACGCCACCCCGTGCCTCCTGCCTCCGCGCAGCCTGCA
GCGGGAGACCCTGTCCCCGCCCCAGCCGTCCTCCTGGGGTGGACCCTAGTTTAATAAAGA
TTCACCAAGTTTCACGC

As used herein, the term “AXL” refers to the gene encoding Tyrosine-protein kinase receptor UFO. The terms “AXL” and “Tyrosine-protein kinase receptor UFO” include wild-type forms of the AXL gene, as well as variants (e.g., splice variants and polymorphisms) of wild-type AXL. Examples of such variants are nucleic acids having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to a wild-type AXL nucleic acid sequence (e.g., SEQ ID NO: 6, ENA accession number M76125). SEQ ID NO: 6 is a wild-type gene sequence encoding AXL protein, and is shown below:

(SEQ ID NO: 6)
GCTGGGCAAAGCCGGTGGCAAGGGCCTCCCCTGCCGCTGTGCCAGGCAGGCAGTGCCAAA
TCCGGGGAGCCTGGAGCTGGGGGGAGGGCCGGGGACAGCCCGGCCCTGCCCCCTCCCCCG
CTGGGAGCCCAGCAACTTCTGAGGAAAGTTTGGCACCCATGGCGTGGCGGTGCCCCAGGA
TGGGCAGGGTCCCGCTGGCCTGGTGCTTGGCGCTGTGCGGCTGGGCGTGCATGGCCCCCA
GGGGCACGCAGGCTGAAGAAAGTCCCTTCGTGGGCAACCCAGGGAATATCACAGGTGCCC
GGGGACTCACGGGCACCCTTCGGTGTCAGCTCCAGGTTCAGGGAGAGCCCCCCGAGGTAC
ATTGGCTTCGGGATGGACAGATCCTGGAGCTCGCGGACAGCACCCAGACCCAGGTGCCCC
TGGGTGAGGATGAACAGGATGACTGGATAGTGGTCAGCCAGCTCAGAATCACCTCCCTGC
AGCTTTCCGACACGGGACAGTACCAGTGTTTGGTGTTTCTGGGACATCAGACCTTCGTGT
CCCAGCCTGGCTATGTTGGGCTGGAGGGCTTGCCTTACTTCCTGGAGGAGCCCGAAGACA
GGACTGTGGCCGCCAACACCCCCTTCAACCTGAGCTGCCAAGCTCAGGGACCCCCAGAGC
CCGTGGACCTACTCTGGCTCCAGGATGCTGTCCCCCTGGCCACGGCTCCAGGTCACGGCC
CCCAGCGCAGCCTGCATGTTCCAGGGCTGAACAAGACATCCTCTTTCTCCTGCGAAGCCC
ATAACGCCAAGGGGGTCACCACATCCCGCACAGCCACCATCACAGTGCTCCCCCAGCAGC
CCCGTAACCTCCACCTGGTCTCCCGCCAACCCACGGAGCTGGAGGTGGCTTGGACTCCAG
GCCTGAGCGGCATCTACCCCCTGACCCACTGCACCCTGCAGGCTGTGCTGTCAGACGATG
GGATGGGCATCCAGGCGGGAGAACCAGACCCCCCAGAGGAGCCCCTCACCTCGCAAGCAT
CCGTGCCCCCCCATCAGCTTCGGCTAGGCAGCCTCCATCCTCACACCCCTTATCACATCC
GCGTGGCATGCACCAGCAGCCAGGGCCCCTCATCCTGGACCCACTGGCTTCCTGTGGAGA
CGCCGGAGGGAGTGCCCCTGGGCCCCCCTAAGAACATTAGTGCTACGCGGAATGGGAGCC
AGGCCTTCGTGCATTGGCAAGAGCCCCGGGCGCCCCTGCAGGGTACCCTGTTAGGGTACC
GGCTGGCGTATCAAGGCCAGGACACCCCAGAGGTGCTAATGGACATAGGGCTAAGGCAAG
AGGTGACCCTGGAGCTGCAGGGGGACGGGTCTGTGTCCAATCTGACAGTGTGTGTGGCAG
CCTACACTGCTGCTGGGGATGGACCCTGGAGCCTCCCAGTACCCCTGGAGGCCTGGCGCC
CAGTGAAGGAACCTTCAACTCCTGCCTTCTCGTGGCCCTGGTGGTATGTACTGCTAGGAG
CAGTCGTGGCCGCTGCCTGTGTCCTCATCTTGGCTCTCTTCCTTGTCCACCGGCGAAAGA
AGGAGACCCGTTATGGAGAAGTGTTTGAACCAACAGTGGAAAGAGGTGAACTGGTAGTCA
GGTACCGCGTGCGCAAGTCCTACAGTCGTCGGACCACTGAAGCTACCTTGAACAGCCTGG
GCATCAGTGAAGAGCTGAAGGAGAAGCTGCGGGATGTGATGGTGGACCGGCACAAGGTGG
CCCTGGGGAAGACTCTGGGAGAGGGAGAGTTTGGAGCTGTGATGGAAGGCCAGCTCAACC
AGGACGACTCCATCCTCAAGGTGGCTGTGAAGACGATGAAGATTGCCATCTGCACGAGGT
CAGAGCTGGAGGATTTCCTGAGTGAAGCGGTCTGCATGAAGGAATTTGACCATCCCAACG
TCATGAGGCTCATCGGTGTCTGTTTCCAGGGTTCTGAACGAGAGAGCTTCCCAGCACCTG
TGGTCATCTTACCTTTCATGAAACATGGAGACCTACACAGCTTCCTCCTCTATTCCCGGC
TCGGGGACCAGCCAGTGTACCTGCCCACTCAGATGCTAGTGAAGTTCATGGCAGACATCG
CCAGTGGCATGGAGTATCTGAGTACCAAGAGATTCATACACCGGGACCTGGCGGCCAGGA
ACTGCATGCTGAATGAGAACATGTCCGTGTGTGTGGCGGACTTCGGGCTCTCCAAGAAGA
TCTACAATGGGGACTACTACCGCCAGGGACGTATCGCCAAGATGCCAGTCAAGTGGATTG
CCATTGAGAGTCTAGCTGACCGTGTCTACACCAGCAAGAGCGATGTGTGGTCCTTCGGGG
TGACAATGTGGGAGATTGCCACAAGAGGCCAAACCCCATATCCGGGCGTGGAGAACAGCG
AGATTTATGACTATCTGCGCCAGGGAAATCGCCTGAAGCAGCCTGCGGACTGTCTGGATG
GACTGTATGCCTTGATGTCGCGGTGCTGGGAGCTAAATCCCCAGGACCGGCCAAGTTTTA
CAGAGCTGCGGGAAGATTTGGAGAACACACTGAAGGCCTTGCCTCCTGCCCAGGAGCCTG
ACGAAATCCTCTATGTCAACATGGATGAGGGTGGAGGTTATCCTGAACCCCCTGGAGCTG
CAGGAGGAGCTGACCCCCCAACCCAGCCAGACCCTAAGGATTCCTGTAGCTGCCTCACTG
CGGCTGAGGTCCATCCTGCTGGACGCTATGTCCTCTGCCCTTCCACAACCCCTAGCCCCG
CTCAGCCTGCTGATAGGGGCTCCCCAGCAGCCCCAGGGCAGGAGGATGGTGCCTGAGACA
ACCCTCCACCTGGTACTCCCTCTCAGGATCCAAGCTAAGCACTGCCACTGGGGAAAACTC
CACCTTCCCACTTTTCCACCCCACGCCTTATCCCCACTTGCAGCCCTGTCTTCCTACCTA
TCCCACCTCCATCCCAGACAGGTCCCTCCCCTTCTCTGTGCAGTAGCATCACCTTGAAAG
CAGTAGCATCACCATCTGTAAAAGGAAGGGGTTGGATTGCAATATCTGAAGCCCTCCCAG
GTGTTAACATTCCAAGACTCTAGAGTCCAAGGTTTAAAGAGTCTAGATTCAAAGGTTCTA
GGTTTCAAAGATGCTGTGAGTCTTTGGTTCTAAGGACCTGAAATTCCAAAGTCTCTAATT
CTATTAAAGTGCTAAGGTTCTAAGGCAAAAAAAAAAAAAAAAAAAAA

As used herein, the term “BIN1” refers to the gene encoding Myc box-dependent-interacting protein 1. The terms “BIN1” and “Myc box-dependent-interacting protein 1” include wild-type forms of the BIN1 gene, as well as variants (e.g., splice variants and polymorphisms) of wild-type BIN1. Examples of such variants are nucleic acids having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to a wild-type BIN1 nucleic acid sequence (e.g., SEQ ID NO: 7, ENA accession number AF004015). SEQ ID NO: 7 is a wild-type gene sequence encoding BIN1 protein, and is shown below:

(SEQ ID NO: 7)
ATGGCAGAGATGGGCAGTAAAGGGGTGACGGCGGGAAAGATCGCCAGCAACGTGCAGAAG
AAGCTCACCCGCGCGCAGGAGAAGGTTCTCCAGAAGCTGGGGAAGGCAGATGAGACCAAG
GATGAGCAGTTTGAGCAGTGCGTCCAGAATTTCAACAAGCAGCTGACGGAGGGCACCCGG
CTGCAGAAGGATCTCCGGACCTACCTGGCCTCCGTCAAAGCCATGCACGAGGCTTCCAAG
AAGCTGAATGAGTGTCTGCAGGAGGTGTATGAGCCCGATTGGCCCGGCAGGGATGAGGCA
AACAAGATCGCAGAGAACAACGACCTGCTGTGGATGGATTACCACCAGAAGCTGGTGGAC
CAGGCGCTGCTGACCATGGACACGTACCTGGGCCAGTTCCCCGACATCAAGTCACGCATT
GCCAAGCGGGGGCGCAAGCTGGTGGACTACGACAGTGCCCGGCACCACTACGAGTCCCTT
CAAACCGCCAAAAAGAAGGATGAAGCCAAAATTGCCAAGCCTGTCTCGCTGCTTGAGAAA
GCCGCCCCCCAGTGGTGCCAAGGCAAACTGCAGGCTCATCTCGTAGCTCAAACTAACCTG
CTCCGAAATCAGGCCGAGGAGGAGCTCATCAAAGCCCAGAAGGTGTTTGAGGAGATGAAT
GTGGATCTGCAGGAGGAGCTGCCGTCCCTGTGGAACAGCCGCGTAGGTTTCTACGTCAAC
ACGTTCCAGAGCATCGCGGGCCTGGAGGAAAACTTCCACAAGGAGATGAGCAAGCTCAAC
CAGAACCTCAATGATGTGCTGGTCGGCCTGGAGAAGCAACACGGGAGCAACACCTTCACG
GTCAAGGCCCAGCCCAGTGACAACGCGCCTGCAAAAGGGAACAAGAGCCCTTCGCCTCCA
GATGGCTCCCCTGCCGCCACCCCCGAGATCAGAGTCAACCACGAGCCAGAGCCGGCCGGC
GGGGCCACGCCCGGGGCCACCCTCCCCAAGTCCCCATCTCAGCTCCGGAAAGGCCCACCA
GTCCCTCCGCCTCCCAAACACACCCCGTCCAAGGAAGTCAAGCAGGAGCAGATCCTCAGC
CTGTTTGAGGACACGTTTGTCCCTGAGATCAGCGTGACCACCCCCTCCCAGTTTGAGGCC
CCGGGGCCTTTCTCGGAGCAGGCCAGTCTGCTGGACCTGGACTTTGACCCCCTCCCGCCC
GTGACGAGCCCTGTGAAGGCACCCACGCCCTCTGGTCAGTCAATTCCATGGGACCTCTGG
GAGCCCACAGAGAGTCCAGCCGGCAGCCTGCCTTCCGGGGAGCCCAGCGCTGCCGAGGGC
ACCTTTGCTGTGTCCTGGCCCAGCCAGACGGCCGAGCCGGGGCCTGCCCAACCAGCAGAG
GCCTCGGAGGTGGCGGGTGGGACCCAACCTGCGGCTGGAGCCCAGGAGCCAGGGGAGACG
GCGGCAAGTGAAGCAGCCTCCAGCTCTCTTCCTGCTGTCGTGGTGGAGACCTTCCCAGCA
ACTGTGAATGGCACCGTGGAGGGCGGCAGTGGGGCCGGGCGCTTGGACCTGCCCCCAGGT
TTCATGTTCAAGGTACAGGCCCAGCACGACTACACGGCCACTGACACAGACGAGCTGCAG
CTCAAGGCTGGTGATGTGGTGCTGGTGATCCCCTTCCAGAACCCTGAAGAGCAGGATGAA
GGCTGGCTCATGGGCGTGAAGGAGAGCGACTGGAACCAGCACAAGGAGCTGGAGAAGTGC
CGTGGCGTCTTCCCCGAGAACTTCACTGAGAGGGTCCCATGA

As used herein, the term “C1QA” refers to the gene encoding Complement C1q A Chain. The terms “C1QA” and “Complement C1q A Chain” include wild-type forms of the C1QA gene, as well as variants (e.g., splice variants and polymorphisms) of wild-type C1QA. Examples of such variants are nucleic acids having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to a wild-type C1QA nucleic acid sequence (e.g., SEQ ID NO: 8, NCBI Reference Sequence: NM_015991.3). SEQ ID NO: 8 is a wild-type gene sequence encoding C1QA protein, and is shown below:

(SEQ ID NO: 8)
AGTCTTGCTGAAGTCTGCTTGAAATGTCCCTGGTGAGCTTCTGGCCACTGGGGAAGTTCAGGGGGC
AGGTCTGAAGAAGGGGAAGTAGGAAGGGATGTGAAACTTGGCCACAGCCTGGAGCCACTCCTGCTG
GGCAGCCCACAGGGTCCCTGGGCGGAGGGCAGGAGCATCCAGTTGGAGTTGACAACAGGAGGCA
GAGGCATCATGGAGGGTCCCCGGGGATGGCTGGTGCTCTGTGTGCTGGCCATATCGCTGGCCTCT
ATGGTGACCGAGGACTTGTGCCGAGCACCAGACGGGAAGAAAGGGGAGGCAGGAAGACCTGGCAG
ACGGGGGGGGCCAGGCCTCAAGGGGGAGCAAGGGGAGCCGGGGGCCCCTGGCATCCGGACAGG
CATCCAAGGCCTTAAAGGAGACCAGGGGGAACCTGGGCCCTCTGGAAACCCCGGCAAGGTGGGCT
ACCCAGGGCCCAGCGGCCCCCTCGGAGCCCGTGGCATCCCGGGAATTAAAGGCACCAAGGGCAGC
CCAGGAAACATCAAGGACCAGCCGAGGCCAGCCTTCTCCGCCATTCGGCGGAACCCCCCAATGGG
GGGCAACGTGGTCATCTTCGACACGGTCATCACCAACCAGGAAGAACCGTACCAGAACCACTCCGG
CCGATTCGTCTGCACTGTACCCGGCTACTACTACTTCACCTTCCAGGTGCTGTCCCAGTGGGAAATC
TGCCTGTCCATCGTCTCCTCCTCAAGGGGCCAGGTCCGACGCTCCCTGGGCTTCTGTGACACCACC
AACAAGGGGCTCTTCCAGGTGGTGTCAGGGGGCATGGTGCTTCAGCTGCAGCAGGGTGACCAGGT
CTGGGTTGAAAAAGACCCCAAAAAGGGTCACATTTACCAGGGCTCTGAGGCCGACAGCGTCTTCAG
CGGCTTCCTCATCTTCCCATCTGCCTGAGCCAGGGAAGGACCCCCTCCCCCACCCACCTCTCTGGC
TTCCATGCTCCGCCTGTAAAATGGGGGCGCTATTGCTTCAGCTGCTGAAGGGAGGGGGCTGGCTCT
GAGAGCCCCAGGACTGGCTGCCCCGTGACACATGCTCTAAGAAGCTCGTTTCTTAGACCTCTTCCTG
GAATAAACATCTGTGTCTGTGTCTGCTGAACATGAGCTTCAGTTGCTACTCGGAGCATTGAGAGGGA
GGCCTAAGAATAATAACAATCCAGTGCTTAAGAGTCAAAAAAAAAAAA

As used herein, the term “C3” refers to the gene encoding Complement C3. The terms “C3” and “Complement C3” include wild-type forms of the C3 gene, as well as variants (e.g., splice variants and polymorphisms) of wild-type C3. Examples of such variants are nucleic acids having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to a wild-type C3 nucleic acid sequence (e.g., SEQ ID NO: 9, NCBI Reference Sequence: NM_000064.3). SEQ ID NO: 9 is a wild-type gene sequence encoding C3 protein, and is shown below:

(SEQ ID NO: 9)
AGATAAAAAGCCAGCTCCAGCAGGCGCTGCTCACTCCTCCCCATCCTCTCCCTCTGTCCCTCTGTCC
CTCTGACCCTGCACTGTCCCAGCACCATGGGACCCACCTCAGGTCCCAGCCTGCTGCTCCTGCTAC
TAACCCACCTCCCCCTGGCTCTGGGGAGTCCCATGTACTCTATCATCACCCCCAACATCTTGCGGCT
GGAGAGCGAGGAGACCATGGTGCTGGAGGCCCACGACGCGCAAGGGGATGTTCCAGTCACTGTTA
CTGTCCACGACTTCCCAGGCAAAAAACTAGTGCTGTCCAGTGAGAAGACTGTGCTGACCCCTGCCA
CCAACCACATGGGCAACGTCACCTTCACGATCCCAGCCAACAGGGAGTTCAAGTCAGAAAAGGGGC
GCAACAAGTTCGTGACCGTGCAGGCCACCTTCGGGACCCAAGTGGTGGAGAAGGTGGTGCTGGTC
AGCCTGCAGAGCGGGTACCTCTTCATCCAGACAGACAAGACCATCTACACCCCTGGCTCCACAGTT
CTCTATCGGATCTTCACCGTCAACCACAAGCTGCTACCCGTGGGCCGGACGGTCATGGTCAACATT
GAGAACCCGGAAGGCATCCCGGTCAAGCAGGACTCCTTGTCTTCTCAGAACCAGCTTGGCGTCTTG
CCCTTGTCTTGGGACATTCCGGAACTCGTCAACATGGGCCAGTGGAAGATCCGAGCCTACTATGAAA
ACTCACCACAGCAGGTCTTCTCCACTGAGTTTGAGGTGAAGGAGTACGTGCTGCCCAGTTTCGAGGT
CATAGTGGAGCCTACAGAGAAATTCTACTACATCTATAACGAGAAGGGCCTGGAGGTCACCATCACC
GCCAGGTTCCTCTACGGGAAGAAAGTGGAGGGAACTGCCTTTGTCATCTTCGGGATCCAGGATGGC
GAACAGAGGATTTCCCTGCCTGAATCCCTCAAGCGCATTCCGATTGAGGATGGCTCGGGGGAGGTT
GTGCTGAGCCGGAAGGTACTGCTGGACGGGGTGCAGAACCCCCGAGCAGAAGACCTGGTGGGGAA
GTCTTTGTACGTGTCTGCCACCGTCATCTTGCACTCAGGCAGTGACATGGTGCAGGCAGAGCGCAG
CGGGATCCCCATCGTGACCTCTCCCTACCAGATCCACTTCACCAAGACACCCAAGTACTTCAAACCA
GGAATGCCCTTTGACCTCATGGTGTTCGTGACGAACCCTGATGGCTCTCCAGCCTACCGAGTCCCC
GTGGCAGTCCAGGGCGAGGACACTGTGCAGTCTCTAACCCAGGGAGATGGCGTGGCCAAACTCAG
CATCAACACACACCCCAGCCAGAAGCCCTTGAGCATCACGGTGCGCACGAAGAAGCAGGAGCTCTC
GGAGGCAGAGCAGGCTACCAGGACCATGCAGGCTCTGCCCTACAGCACCGTGGGCAACTCCAACA
ATTACCTGCATCTCTCAGTGCTACGTACAGAGCTCAGACCCGGGGAGACCCTCAACGTCAACTTCCT
CCTGCGAATGGACCGCGCCCACGAGGCCAAGATCCGCTACTACACCTACCTGATCATGAACAAGGG
CAGGCTGTTGAAGGCGGGACGCCAGGTGCGAGAGCCCGGCCAGGACCTGGTGGTGCTGCCCCTG
TCCATCACCACCGACTTCATCCCTTCCTTCCGCCTGGTGGCGTACTACACGCTGATCGGTGCCAGC
GGCCAGAGGGAGGTGGTGGCCGACTCCGTGTGGGTGGACGTCAAGGACTCCTGCGTGGGCTCGCT
GGTGGTAAAAAGCGGCCAGTCAGAAGACCGGCAGCCTGTACCTGGGCAGCAGATGACCCTGAAGA
TAGAGGGTGACCACGGGGCCCGGGTGGTACTGGTGGCCGTGGACAAGGGCGTGTTCGTGCTGAAT
AAGAAGAACAAACTGACGCAGAGTAAGATCTGGGACGTGGTGGAGAAGGCAGACATCGGCTGCACC
CCGGGCAGTGGGAAGGATTACGCCGGTGTCTTCTCCGACGCAGGGCTGACCTTCACGAGCAGCAG
TGGCCAGCAGACCGCCCAGAGGGCAGAACTTCAGTGCCCGCAGCCAGCCGCCCGCCGACGCCGTT
CCGTGCAGCTCACGGAGAAGCGAATGGACAAAGTCGGCAAGTACCCCAAGGAGCTGCGCAAGTGC
TGCGAGGACGGCATGCGGGAGAACCCCATGAGGTTCTCGTGCCAGCGCCGGACCCGTTTCATCTC
CCTGGGCGAGGCGTGCAAGAAGGTCTTCCTGGACTGCTGCAACTACATCACAGAGCTGCGGCGGC
AGCACGCGCGGGCCAGCCACCTGGGCCTGGCCAGGAGTAACCTGGATGAGGACATCATTGCAGAA
GAGAACATCGTTTCCCGAAGTGAGTTCCCAGAGAGCTGGCTGTGGAACGTTGAGGACTTGAAAGAG
CCACCGAAAAATGGAATCTCTACGAAGCTCATGAATATATTTTTGAAAGACTCCATCACCACGTGGGA
GATTCTGGCTGTGAGCATGTCGGACAAGAAAGGGATCTGTGTGGCAGACCCCTTCGAGGTCACAGT
AATGCAGGACTTCTTCATCGACCTGCGGCTACCCTACTCTGTTGTTCGAAACGAGCAGGTGGAAATC
CGAGCCGTTCTCTACAATTACCGGCAGAACCAAGAGCTCAAGGTGAGGGTGGAACTACTCCACAAT
CCAGCCTTCTGCAGCCTGGCCACCACCAAGAGGCGTCACCAGCAGACCGTAACCATCCCCCCCAAG
TCCTCGTTGTCCGTTCCATATGTCATCGTGCCGCTAAAGACCGGCCTGCAGGAAGTGGAAGTCAAG
GCTGCTGTCTACCATCATTTCATCAGTGACGGTGTCAGGAAGTCCCTGAAGGTCGTGCCGGAAGGA
ATCAGAATGAACAAAACTGTGGCTGTTCGCACCCTGGATCCAGAACGCCTGGGCCGTGAAGGAGTG
CAGAAAGAGGACATCCCACCTGCAGACCTCAGTGACCAAGTCCCGGACACCGAGTCTGAGACCAGA
ATTCTCCTGCAAGGGACCCCAGTGGCCCAGATGACAGAGGATGCCGTCGACGCGGAACGGCTGAA
GCACCTCATTGTGACCCCCTCGGGCTGCGGGGAACAGAACATGATCGGCATGACGCCCACGGTCAT
CGCTGTGCATTACCTGGATGAAACGGAGCAGTGGGAGAAGTTCGGCCTAGAGAAGCGGCAGGGGG
CCTTGGAGCTCATCAAGAAGGGGTACACCCAGCAGCTGGCCTTCAGACAACCCAGCTCTGCCTTTG
CGGCCTTCGTGAAACGGGCACCCAGCACCTGGCTGACCGCCTACGTGGTCAAGGTCTTCTCTCTGG
CTGTCAACCTCATCGCCATCGACTCCCAAGTCCTCTGCGGGGCTGTTAAATGGCTGATCCTGGAGAA
GCAGAAGCCCGACGGGGTCTTCCAGGAGGATGCGCCCGTGATACACCAAGAAATGATTGGTGGATT
ACGGAACAACAACGAGAAAGACATGGCCCTCACGGCCTTTGTTCTCATCTCGCTGCAGGAGGCTAA
AGATATTTGCGAGGAGCAGGTCAACAGCCTGCCAGGCAGCATCACTAAAGCAGGAGACTTCCTTGA
AGCCAACTACATGAACCTACAGAGATCCTACACTGTGGCCATTGCTGGCTATGCTCTGGCCCAGATG
GGCAGGCTGAAGGGGCCTCTTCTTAACAAATTTCTGACCACAGCCAAAGATAAGAACCGCTGGGAG
GACCCTGGTAAGCAGCTCTACAACGTGGAGGCCACATCCTATGCCCTCTTGGCCCTACTGCAGCTA
AAAGACTTTGACTTTGTGCCTCCCGTCGTGCGTTGGCTCAATGAACAGAGATACTACGGTGGTGGCT
ATGGCTCTACCCAGGCCACCTTCATGGTGTTCCAAGCCTTGGCTCAATACCAAAAGGACGCCCCTGA
CCACCAGGAACTGAACCTTGATGTGTCCCTCCAACTGCCCAGCCGCAGCTCCAAGATCACCCACCG
TATCCACTGGGAATCTGCCAGCCTCCTGCGATCAGAAGAGACCAAGGAAAATGAGGGTTTCACAGTC
ACAGCTGAAGGAAAAGGCCAAGGCACCTTGTCGGTGGTGACAATGTACCATGCTAAGGCCAAAGAT
CAACTCACCTGTAATAAATTCGACCTCAAGGTCACCATAAAACCAGCACCGGAAACAGAAAAGAGGC
CTCAGGATGCCAAGAACACTATGATCCTTGAGATCTGTACCAGGTACCGGGGAGACCAGGATGCCA
CTATGTCTATATTGGACATATCCATGATGACTGGCTTTGCTCCAGACACAGATGACCTGAAGCAGCT
GGCCAATGGTGTTGACAGATACATCTCCAAGTATGAGCTGGACAAAGCCTTCTCCGATAGGAACACC
CTCATCATCTACCTGGACAAGGTCTCACACTCTGAGGATGACTGTCTAGCTTTCAAAGTTCACCAATA
CTTTAATGTAGAGCTTATCCAGCCTGGAGCAGTCAAGGTCTACGCCTATTACAACCTGGAGGAAAGC
TGTACCCGGTTCTACCATCCGGAAAAGGAGGATGGAAAGCTGAACAAGCTCTGCCGTGATGAACTG
TGCCGCTGTGCTGAGGAGAATTGCTTCATACAAAAGTCGGATGACAAGGTCACCCTGGAAGAACGG
CTGGACAAGGCCTGTGAGCCAGGAGTGGACTATGTGTACAAGACCCGACTGGTCAAGGTTCAGCTG
TCCAATGACTTTGACGAGTACATCATGGCCATTGAGCAGACCATCAAGTCAGGCTCGGATGAGGTGC
AGGTTGGACAGCAGCGCACGTTCATCAGCCCCATCAAGTGCAGAGAAGCCCTGAAGCTGGAGGAGA
AGAAACACTACCTCATGTGGGGTCTCTCCTCCGATTTCTGGGGAGAGAAGCCCAACCTCAGCTACAT
CATCGGGAAGGACACTTGGGTGGAGCACTGGCCCGAGGAGGACGAATGCCAAGACGAAGAGAACC
AGAAACAATGCCAGGACCTCGGCGCCTTCACCGAGAGCATGGTTGTCTTTGGGTGCCCCAACTGAC
CACACCCCCATTCCCCCACTCCAGATAAAGCTTCAGTTATATCTCAAAAAAAAAAAAAAAAA

As used herein, the term “C9orf72” refers to the gene encoding Guanine nucleotide exchange C9orf72. The terms “C9orf72” and “Guanine nucleotide exchange C9orf72” include wild-type forms of the C9orf72 gene, as well as variants (e.g., splice variants and polymorphisms) of wild-type C9orf72. Examples of such variants are nucleic acids having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to a wild-type C9orf72 nucleic acid sequence (e.g., SEQ ID NO: 10, ENA accession number JN681271). SEQ ID NO: 10 is a wild-type gene sequence encoding C9orf72 protein, and is shown below:

(SEQ ID NO: 10)
AGGAAAGAGAGGTGCGTCAAACAGCGACAAGTTCCGCCCACGTAAAAGATGACGCTTGGT
GTGTCAGCCGTCCCTGCTGCCCGGTTGCTTCTCTTTTGGGGGCGGGGTCTAGCAAGAGCA
GGTGTGGGTTTAGGAGATATCTCCGGAGCATTTGGATAATGTGACAGTTGGAATGCAGTG
ATGTCGACTCTTTGCCCACCGCCATCTCCAGCTGTTGCCAAGACAGAGATTGCTTTAAGT
GGCAAATCACCTTTATTAGCAGCTACTTTTGCTTACTGGGACAATATTCTTGGTCCTAGA
GTAAGGCACATTTGGGCTCCAAAGACAGAACAGGTACTTCTCAGTGATGGAGAAATAACT
TTTCTTGCCAACCACACTCTAAATGGAGAAATCCTTCGAAATGCAGAGAGTGGTGCTATA
GATGTAAAGTTTTTTGTCTTGTCTGAAAAGGGAGTGATTATTGTTTCATTAATCTTTGAT
GGAAACTGGAATGGGGATCGCAGCACATATGGACTATCAATTATACTTCCACAGACAGAA
CTTAGTTTCTACCTCCCACTTCATAGAGTGTGTGTTGATAGATTAACACATATAATCCGG
AAAGGAAGAATATGGATGCATAAGGAAAGACAAGAAAATGTCCAGAAGATTATOTTAGAA
GGCACAGAGAGAATGGAAGATCAGGGTCAGAGTATTATTCCAATGCTTACTGGAGAAGTG
ATTCCTGTAATGGAACTGCTTTCATCTATGAAATCACACAGTGTTCCTGAAGAAATAGAT
ATAGCTGATACAGTACTCAATGATGATGATATTGGTGACAGCTGTCATGAAGGCTTTCTT
CTCAATGCCATCAGCTCACACTTGCAAACCTGTGGCTGTTCCGTTGTAGTAGGTAGCAGT
GCAGAGAAAGTAAATAAGATAGTCAGAACATTATGCCTTTTTCTGACTCCAGCAGAGAGA
AAATGCTCCAGGTTATGTGAAGCAGAATCATCATTTAAATATGAGTCAGGGCTCTTTGTA
CAAGGCCTGCTAAAGGATTCAACTGGAAGCTTTGTGCTGCCTTTCCGGCAAGTCATGTAT
GCTCCATATCCCACCACACACATAGATGTGGATGTCAATACTGTGAAGCAGATGCCACCC
TGTCATGAACATATTTATAATCAGCGTAGATACATGAGATCCGAGCTGACAGCCTTCTGG
AGAGCCACTTCAGAAGAAGACATGGCTCAGGATACGATCATCTACACTGACGAAAGCTTT
ACTCCTGATTTGAATATTTTTCAAGATGTCTTACACAGAGACACTCTAGTGAAAGCCTTC
CTGGATCAGGTCTTTCAGCTGAAACCTGGCTTATCTCTCAGAAGTACTTTCCTTGCACAG
TTTCTACTTGTCCTTCACAGAAAAGCCTTGACACTAATAAAATATATAGAAGACGATACG
CAGAAGGGAAAAAAGCCCTTTAAATCTCTTCGGAACCTGAAGATAGACCTTGATTTAACA
GCAGAGGGCGATCTTAACATAATAATGGCTCTGGCTGAGAAAATTAAACCAGGCCTACAC
TCTTTTATCTTTGGAAGACCTTTCTACACTAGTGTGCAAGAACGAGATGTTCTAATGACT
TTTTAAATGTGTAACTTAATAAGCCTATTCCATCACAATCATGATCGCTGGTAAAGTAGC
TCAGTGGTGTGGGGAAACGTTCCCCTGGATCATACTCCAGAATTCTGCTCTCAGCAATTG
CAGTTAAGTAAGTTACACTACAGTTCTCACAAGAGCCTGTGAGGGGATGTCAGGTGCATC
ATTACATTGGGTGTCTCTTTTCCTAGATTTATGCTTTTGGGATACAGACCTATGTTTACA
ATATAATAAATATTATTGCTATCTTTTAAAGATATAATAATAGGATGTAAACTTGACCAC
AACTACTGTTTTTTTGAAATACATGATTCATGGTTTACATGTGTCAAGGTGAAATCTGAG
TTGGCTTTTACAGATAGTTGACTTTCTATCTTTTGGCATTCTTTGGTGTGTAGAATTACT
GTAATACTTCTGCAATCAACTGAAAACTAGAGCCTTTAAATGATTTCAATTCCACAGAAA
GAAAGTGAGCTTGAACATAGGATGAGCTTTAGAAAGAAAATTGATCAAGCAGATGTTTAA
TTGGAATTGATTATTAGATCCTACTTTGTGGATTTAGTCCCTGGGATTCAGTCTGTAGAA
ATGTCTAATAGTTCTCTATAGTCCTTGTTCCTGGTGAACCACAGTTAGGGTGTTTTGTTT
ATTTTATTGTTCTTGCTATTGTTGATATTCTATGTAGTTGAGCTCTGTAAAAGGAAATTG
TATTTTATGTTTTAGTAATTGTTGCCAACTTTTTAAATTAATTTTCATTATTTTTGAGCC
AAATTGAAATGTGCACCTCCTGTGCCTTTTTTCTCCTTAGAAAATCTAATTACTTGGAAC
AAGTTCAGATTTCACTGGTCAGTCATTTTCATCTTGTTTTCTTCTTGCTAAGTCTTACCA
TGTACCTGCTTTGGCAATCATTGCAACTCTGAGATTATAAAATGCCTTAGAGAATATACT
AACTAATAAGATCTTTTTTTCAGAAACAGAAAATAGTTCCTTGAGTACTTCCTTCTTGCA
TTTCTGCCTATGTTTTTGAAGTTGTTGCTGTTTGCCTGCAATAGGCTATAAGGAATAGCA
GGAGAAATTTTACTGAAGTGCTGTTTTCCTAGGTGCTACTTTGGCAGAGCTAAGTTATCT
TTTGTTTTCTTAATGCGTTTGGACCATTTTGCTGGCTATAAAATAACTGATTAATATAAT
TCTAACACAATGTTGACATTGTAGTTACACAAACACAAATAAATATTTTATTTAAAATTC
TGGAAGTAATATAAAAGGGAAAATATATTTATAAGAAAGGGATAAAGGTAATAGAGCCCT
TCTGCCCCCCACCCACCAAATTTACACAACAAAATGACATGTTCGAATGTGAAAGGTCAT
AATAGCTTTCCCATCATGAATCAGAAAGATGTGGACAGCTTGATGTTTTAGACAACCACT
GAACTAGATGACTGTTGTACTGTAGCTCAGTCATTTAAAAAATATATAAATACTACCTTG
TAGTGTCCCATACTGTGTTTTTTACATGGTAGATTCTTATTTAAGTGCTAACTGGTTATT
TTCTTTGGCTGGTTTATTGTACTGTTATACAGAATGTAAGTTGTACAGTGAAATAAGTTA
TTAAAGCATGTGTAAACATTGTTATATATCTTTTCTCCTAAATGGAGAATTTTGAATAAA
ATATATTTGAAATTTT

As used herein, the term “CASS4” refers to the gene encoding Cas scaffolding protein family member 4. The terms “CASS4” and “Cas scaffolding protein family member 4” include wild-type forms of the CASS4 gene, as well as variants (e.g., splice variants and polymorphisms) of wild-type CASS4. Examples of such variants are nucleic acids having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to a wild-type CASS4 nucleic acid sequence (e.g., SEQ ID NO: 11, ENA accession number AJ276678). SEQ ID NO: 11 is a wild-type gene sequence encoding CASS4 protein, and is shown below:

GAAGAGTGGTGTTTTTTTCTTCTTCTTCTTCTTTTGTGGTTTCACATAGCAAATGAGTGA
CAGTCTCTACTTACAGACAAAGTGAGACGTCAGGCATTGAGACATAGCTCCATAGAATTC
AGTTTCTGAGAACCAGCCAGAAGCATGCAGTGACATTGCACAATCTGCCTCTGAAGCTGG
AGATACTAGCTGCAGAGCTCAGGGGAGCTGCTCCACATCACCGACATGAAGGGAACAGGC
ATCATGGACTGTGCGCCCAAGGCACTCCTGGCCAGGGCACTTTATGACAACTGCCCTGAC
TGCTCTGACGAGCTGGCTTTCAGCAGAGGGGACATCCTGACCATTCTGGAGCAACACGTG
CCAGAAAGCGAGGGTTGGTGGAAGTGTTTGCTCCATGGGAGGCAAGGCCTGGCCCCTGCC
AACCGCCTCCAAATCCTCACGGAGGTCGCTGCAGACAGGCCGTGCCCCCCATTCCTGAGA
GGCCTGGAAGAAGCTCCTGCCAGCTCAGAGGAGACCTATCAGGTGCCCACTCTACCCCGC
CCTCCCACTCCAGGCCCCGTTTATGAGCAGATGAGGAGTTGGGGGGAGGGGCCCCAGCCC
CCTACTGCCCAAGTCTATGAATTCCCCGACCCTCCCACCAGTGCCAGAATCATCTGTGAA
AAGACTCTCAGCTTTCCAAAACAGGCCATCCTCACGCTTCCCAGACCTGTCCGGGCCTCA
CTGCCGACTCTGCCTTCCCAGGTGTATGACGTGCCTACCCAGCACCGGGGCCCCGTGGTC
CTGAAGGAGCCAGAGAAGCAGCAGTTATATGACATACCAGCCAGCCCCAAGAAGGCAGGA
CTCCATCCCCCAGACAGCCAAGCAAGTGGGCAGGGTGTTCCCCTGATATCAGTGACTACC
TTAAGAAGAGGCGGTTACAGCACATTACCAAATCCTCAGAAATCGGAATGGATTTATGAC
ACTCCAGTGTCTCCAGGAAAGGCCAGCGTCAGAAACACGCCTCTCACCAGCTTTGCGGAA
GAATCAAGGCCCCACGCTCTCCCCAGTTCCAGCTCCACTTTCTACAATCCTCCAAGTGGC
AGATCCAGGTCCCTCACTCCACAACTGAATAACAATGTGCCCATGCAGAAAAAACTCAGC
CTTCCAGAAATTCCTTCTTATGGCTTTCTTGTACCCAGAGGCACATTTCCTTTGGATGAA
GATGTCAGCAACAAGGTTCCTTCAAGCTTCTCTGATTCCCCGAGTGGACAGCAGAACACC
AAGCCCAATATAGACATCCCTAAAGCAACGTCGAGTGTTTCTCAGGCTGGGAAGGAGCTG
GAGAAAGCCAAGGAGGTGTCAGAGAATTCCGCGGGCCATAATTCCTCATGGTTCTCCAGA
CGGACAACTTCCCCATCTCCTGAACCGGACAGATTATCAGGTTCCAGTTCTGACAGCAGA
GCTAGCATCGTTTCCTCGTGCTCCACCACATCCACCGACGACTCCTCCAGCTCTTCCTCG
GAGGAGTCAGCAAAGGAGCTCTCCTTGGACCTGGATGTGGCCAAGGAGACAGTGATGGCT
CTGCAGCACAAGGTGGTCAGCTCTGTCGCTGGCCTGATGCTCTTTGTCAGCAGGAAGTGG
AGATTCCGAGACTATCTGGAGGCCAACATTGATGCAATCCACAGGTCCACTGATCACATA
GAAGCCTCTGTAAGAGAATTTCTGGATTTTGCCCGAGGAGTCCATGGGACTGCCTGTAAC
CTCACTGACAGTAACCTTCAGAACAGAATTCGGGACCAGATGCAGACCATCTCCAACTCC
TACCGCATCCTGCTTGAAACAAAGGAAAGCTTGGATAATCGCAATTGGCCTCTGGAAGTT
CTTGTGACTGACAGTGTCCAGAACAGCCCAGATGACCTTGAGAGGTTTGTCATGGTGGCA
CGGATGCTTCCAGAAGACATCAAGAGGTTTGCCTCCATTGTCATTGCCAATGGAAGGCTC
CTTTTTAAGCGGAACTGTGAAAAGGAAGAGACTGTGCAGTTGACCCCAAATGCAGAATTT
AAGTGTGAAAAATACATCCAGCCTCCCCAAAGAGAAACTGAATCACACCAAAAGAGTACC
CCTTCCACTAAGCAAAGGGAAGATGAACACTCTTCTGAACTATTAAAGAAAAATAGAGCA
AATATCTGTGGACAGAATCCTGGCCCTCTTATACCTCAGCCTTCGAGTCAACAGACTCCT
GAGAGGAAACCCCGCTTATCTGAACACTGCCGGCTGTACTTTGGGGCGCTCTTCAAAGCC
ATCAGCGCATTTCACGGCAGCCTCAGCAGCAGCCAGCCCGCGGAGATCATCACTCAGAGC
AAGCTGGTCATCATGGTGGGACAGAAGCTGGTGGACACGCTGTGCATGGAGACCCAGGAG
AGGGACGTGCGCAATGAGATCCTCCGCGGCAGCAGTCACCTCTGCAGCCTGCTCAAGGAC
GTAGCGCTGGCCACTAAGAATGCCGTGCTCACATACCCCAGCCCTGCCGCGCTGGGGCAC
CTCCAGGCGGAGGCTGAGAAGCTGGAGCAACACACGCGGCAGTTCAGAGGGACACTGGGA
TGAGGACTGTCTACCTCCCTTCCTCCTCTGCTCACC

### (SEQ ID NO: 11)

As used herein, the term “CCL5” refers to the gene encoding C-C motif chemokine 5. The terms “CCL5” and “C-C motif chemokine 5” include wild-type forms of the CCL5 gene, as well as variants (e.g., splice variants and polymorphisms) of wild-type CCL5. Examples of such variants are nucleic acids having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to a wild-type CCL5 nucleic acid sequence (e.g., SEQ ID NO: 12, ENA accession number M21121). SEQ ID NO: 12 is a wild-type gene sequence encoding CCL5 protein, and is shown below:

(SEQ ID NO: 12)
CCTCCGACAGCCTCTCCACAGGTACCATGAAGGTCTCCGCGGCACGCCTCGCTGTCATCC
TCATTGCTACTGCCCTCTGCGCTCCTGCATCTGCCTCCCCATATTCCTCGGACACCACAC
CCTGCTGCTTTGCCTACATTGCCCGCCCACTGCCCCGTGCCCACATCAAGGAGTATTTCT
ACACCAGTGGCAAGTGCTCCAACCCAGCAGTCGTCTTTGTCACCCGAAAGAACCGCCAAG
TGTGTGCCAACCCAGAGAAGAAATGGGTTCGGGAGTACATCAACTCTTTGGAGATGAGCT
AGGATGGAGAGTCCTTGAACCTGAACTTACACAAATTTGCCTGTTTCTGCTTGCTCTTGT
CCTAGCTTGGGAGGCTTCCCCTCACTATCCTACCCCACCCGCTCCTTGAAGGGCCCAGAT
TCTGACCACGACGAGCAGCAGTTACAAAAACCTTCCCCAGGCTGGACGTGGTGGCTCAGC
CTTGTAATCCCAGCACTTTGGGAGGCCAAGGTGGGTGGATCACTTGAGGTCAGGAGTTCG
AGACAGCCTGGCCAACATGATGAAACCCCATGTGTACTAAAAATACAAAAAATTAGCCGG
GCGTGGTAGCGGGCGCCTGTAGTCCCAGCTACTCGGGAGGCTGAGGCAGGAGAATGGCGT
GAACCCGGGAGCGGAGCTTGCAGTGAGCCGAGATCGCGCCACTGCACTCCAGCCTGGGCG
ACAGAGCGAGACTCCGTCTCAAAAAAAAAAAAAAAAAAAAAAAAAATACAAAAATTAGCC
GCGTGGTGGCCCACGCCTGTAATCCCAGCTACTCGGGAGGCTAAGGCAGGAAAATTGTTT
GAACCCAGGAGGTGGAGGCTGCAGTGAGCTGAGATTGTGCCACTTCACTCCAGCCTGGGT
GACAAAGTGAGACTCCGTCACAACAACAACAACAAAAAGCTTCCCCAACTAAAGCCTAGA
AGAGCTTCTGAGGCGCTGCTTTGTCAAAAGGAAGTCTCTAGGTTCTGAGCTCTGGCTTTG
CCTTGGCTTTGCAAGGGCTCTGTGACAAGGAAGGAAGTCAGCATGCCTCTAGAGGCAAGG
AAGGGAGGAACACTGCACTCTTAAGCTTCCGCCGTCTCAACCCCTCACAGGAGCTTACTG
GCAAACATGAAAAATCGGGG

As used herein, the term “CD2AP” refers to the gene encoding CD2-associated protein. The terms “CD2AP” and “CD2-associated protein” include wild-type forms of the CD2AP gene, as well as variants (e.g., splice variants and polymorphisms) of wild-type CD2AP. Examples of such variants are nucleic acids having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to a wild-type CD2AP nucleic acid sequence (e.g., SEQ ID NO: 13, ENA accession number AF146277). SEQ ID NO: 13 is a wild-type gene sequence encoding CD2AP protein, and is shown below:

(SEQ ID NO: 13)
GGAATTCCGGGAGGAGCGGACGTCGGCTTCTCCCCGCGGGAGCCCCCAGCATGGTTGACT
ATATTGTGGAGTATGACTATGATGCTGTACATGATGATGAATTAACTATTCGAGTTGGAG
AAATCATCAGGAATGTGAAAAAGCTACAGGAGGAAGGGTGGCTGGAAGGAGAACTAAATG
GGAGAAGAGGAATGTTCCCTGACAATTTCGTTAAGGAAATTAAAAGAGAGACGGAATTCA
AGGATGACAGTTTGCCCATCAAACGGGAAAGGCATGGGAATGTAGCAAGTCTTGTACAAC
GAATAAGCACCTATGGACTTCCAGCTGGAGGAATTCAGCCACATCCACAAACCAAAAACA
TTAAGAAGAAGACCAAGAAGCGTCAGTGTAAAGTTCTTTTTGAGTACATTCCACAAAATG
AGGATGAACTGGAGCTGAAAGTGGGAGATATTATTGATATTAATGAAGAGGTAGAAGAAG
GCTGGTGGAGTGGAACCCTGAATAACAAGTTGGGACTGTTTCCCTCAAATTTTGTGAAAG
AATTAGAGGTAACAGATGATGGTGAAACTCATGAAGCCCAGGACGATTCAGAAACTGTTT
TGGCTGGGCCTACTTCACCTATACCTTCTCTGGGAAATGTGAGTGAAACTGCATCTGGAT
CAGTTACACAGCCAAAGAAAATTCGAGGAATTGGATTTGGAGACATTTTTAAAGAAGGTT
CTGTGAAACTTCGGACAAGAACATCCAGTAGTGAAACAGAAGAGAAAAAACCAGAAAAGC
CCTTAATCCTACAGTCACTGGGACCCAAAACTCAGAGTGTGGAGATAACAAAAACAGATA
CCGAAGGTAAAATTAAAGCTAAAGAATATTGTAGAACATTATTTGCCTATGAAGGTACTA
ATGAAGATGAACTTACTTTTAAAGAGGGGGAGATAATCCATTTGATAAGTAAGGAGACTG
GAGAAGCTGGCTGGTGGAGGGGCGAACTTAATGGTAAAGAAGGAGTATTTCCAGACAATT
TTGCTGTCCAGATAAATGAACTTGATAAAGACTTTCCAAAACCAAAGAAACCACCACCTC
CTGCTAAGGCTCCAGCTCCAAAGCCTGAACTGATAGCTGCAGAGAAGAAATATTTTTCTT
TAAAGCCTGAAGAAAAGGATGAAAAATCAACACTGGAACAGAAACCTTCTAAACCAGCAG
CTCCACAAGTCCCACCCAAGAAACCTACTCCACCTACCAAAGCCAGTAATTTATTGAGAT
CTTCTGGAACAGTGTACCCAAAGCGACCTGAAAAACCAGTTCCTCCACCACCTCCTATAG
CCAAGATTAATGGGGAAGTTTCTAGCATTTCATCAAAATTTGAAACTGAGCCAGTATCAA
AACTAAAGCTAGATTCTGAACAGCTGCCCCTTAGACCAAAATCAGTAGACTTTGATTCAC
TTACAGTAAGGACCTCCAAAGAAACAGATGTTGTAAATTTTGATGACATAGCTTCCTCAG
AAAACTTGCTTCATCTCACTGCAAATAGACCAAAGATGCCTGGAAGAAGGTTGCCGGGCC
GTTTCAATGGTGGACATTCTCCAACTCACAGCCCCGAAAAAATCTTGAAGTTACCAAAAG
AAGAAGACAGTGCCAACCTGAAGCCATCTGAATTAAAAAAAGATACATGCTACTCTCCAA
AGCCATCTGTGTACCTTTCAACACCTTCCAGTGCTTCTAAAGCAAATACAACTGCTTTCC
TGACTCCATTAGAAATCAAAGCTAAAGTGGAAACAGATGATGTGAAAAAAAATTCCCTGG
ATGAACTTAGAGCCCAGATTATTGAATTGTTGTGCATTGTAGAAGCACTGAAAAAGGATC
ACGGGAAAGAACTGGAAAAACTGCGAAAAGATTTGGAAGAAGAGAAGACAATGAGAAGTA
ATCTAGAGATGGAAATAGAGAAGCTGAAAAAAGCTGTCCTGTCTTCTTGAGTGGTGTGGA
CCTGGTGTTCATAATGTTCCAGGGATTCAGAAGCAACGCTATGAACTTCAGCTGACTTGT
TACTTAAAAATTGTGAATTCTGTTGTTGTGATAAATATGAGCAAATGAAGTGTAATATCT
ATAGAAAAGTAGAGTGAGGGTGAATTTATATATATATTTTGTTTTGCCAATATGAAGAAA
AAGAGGCCTTATTTCTTAACTGTGCTGGGATTGCAAACACTTTTTAAAAAATTGTTTGCT
TGAAAATACTACTGAATATAAATAAGAATGTGCTCAGTAGTTTTTTTATTGAAACTTGTA
TTATTTTTAAAGAGATCTATACTATAAATATGGTGATATATTTACAAGTAATCTGTAAGA
TATACTATTTGAGAGGGACAGATTAGCCTTTTAGTAACTATAGTCACTACTTTTTCCATA
ATGCATAAGGGATATAAACTCTGTGTGTGTGTGTGTGTGTGTGTGTGTGTGTGTGTGTGT
ATATATATATATATATTTTTACTTTTATCCTCTTACCGAAGGTTACACTGTTGTGCCTGT
TTGTCTGCAATGCTGTTTATATTTTGGGTGATGAAATAGGAGTTTCCTAGCTATATAAAC
CAGATTACTCACCCATGCATATAGTAAGAACTAATGAATAATCAAAATAATTTCATCAAC
TTTTAGAATATTTTATGTTGCTTGCACTATAGGAGTCATAAAAGGAACTTAGTTAAAATA
TGTTGGATTGTTAAACATTTGGGGAAATATGAACTGTATTTTAAATTTGTTAGGTCTGAA
AAATCTAAAACTGTTAATTTAACCCTTAACTTGTGCCTAGAAACTACAGCACATATAAAA
TATGTAAACACCAGCCTGTTGCTGTACTTTTCTGCTTATTTTACAGCCTCAAATATTTCT
CATTATCTTGTCACTTAGTTCTTCATGTTTCTCCTTCTGACTTTTAATAATGGTAATAGG
AAAACAAAACCCAAAGCTTTTCAAACTTCAGTGTGAGGTTTCCTATTTTGACAAGTTAAC
TTGTAAATACTCAGGTTTTACGATGTATAATTTACCTAATAGACCAAACTAACTCATGGA
GATATTTTGAACTATTATTTAGGTACAAACTTTATAAAGAATGTTAGTATGTCATAAAAT
ATAACATTACAGCTTATTTAAAACCAAATATATTGAACATATTTTAAAATACATTTCACA
GAATGGATGAATTAGTTGTTTCTTCAAAAGTTACTTATGAACAGTTGAATGCCTTTAAAA
TGTTCTGTCTGTAGGTACATCTAAAAACACAAGTGGGTTTATTTAAATTTTTAAAATTTG
AAATTTTTTATTTGCCAAAAATTGTTTTATGCTTTATTATATCGCAAATGAGTGTCAGAT
TTTTGAGTACCAATGATCATGCTTCCATTTTTTTTAGTTTTAAACCACCAAACCAATATT
TTTCCTTTAAATTTTAATCTTATAATATAGAAATCTTATGTTAATGAAATTTTGTCATGT
TTCAAATAAAGAAAACTGAAGTAGAAAATAGAAATGCCAGTAAACAACATAATGTTTAAT
TTACAACTTACATTAGGGGTTTGGGGGAATGCTAATTATATATTGAGAATATACATTAGA
ACTCTTCAAAATGGGCTCTTCTAATGAGGTCACTACTGAACAAAATTGTTCCCTCTTCTG
TTAAATAGAATAGGTTTAAATGACTAGTCAAATGAATTATTTTCTCCTTGTTAAATAAAT
TAAATCTTACTTTCTTTTAATGACCAACCTTAGGTAAAACAAAAATATTGTAATCCTAGA
AATTATCCTCCAGCTTTCTCACCTGAAAATCTATTGAAGTGATCCCTGGTCATCCTAATA
ATGGGATGAGGGAAGTTTCCAGCAGATTTCAGGCTGTTCTTAAAGTTTTTGTTGGTCATT
TTCTCAATAGTACATGAAATCAAGATGCTTATGAGCATGGAAATGTATTTAAAGTTTTTG
CTTGTGTCCTCCTCAGTCAGAATAGAAAAGTAACTGAAATACTCTTACCTTTCTGTCCTT
GATAAAATAGTAAAGAAAACCAAACAAACCCAGGCCTGATGGGAAAAATGATTCCTTTAT
TCTAGCAATTACTTTCTGTTGGTATGGGAAATGTTATTAATTTCTATTACTAAAGTTCAT
ATCACAAAATGATATTTAATAATAACCTTGGGGTAAATCATGAATTTTTTTTTCTACGTG
TGAGTATAAAAGACAAAAGTTGAACAGCATGGAATCTTCATTGCCAAATTATTAGTGAAT
GTATAGTTCAGGTATTCTTTGAGACACACAGTATCATTAATTTCCGAATTGTATTTCAGT
GTTATTTTTTGTTTGTGACCACTAAGCTTCTGTCTTAATACAAAGCTGTTACCTTCTACA
GAATTTAAGTCTGAAGATGTAAAGAGAGAACAGGCCTTGTGTAACAGAAGATACTCTTTT
TTATGCTCCTTACTGTGATCACAGAAAAATTAAAAATCCAAGTGCTCTCTAGATTTGTTG
ATAAACATTTTATGCTTGCATTTAAACTTGAAATGTATGAGCAGAATGAGACAATCAGTT
AAATCAGAAATGAGAAGTATTATAATGTAAAGGCCTTGTTTTGCTGTAGCAATAAAATGA
CCAAGTGCAATGACTTGATTTAATAAAATCCGGAATTC

As used herein, the term “CD33” refers to the gene encoding Myeloid cell surface antigen CD33. The terms “CD33” and “Myeloid cell surface antigen CD33” include wild-type forms of the CD33 gene, as well as variants (e.g., splice variants and polymorphisms) of wild-type CD33. Examples of such variants are nucleic acids having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to a wild-type CD33 nucleic acid sequence (e.g., SEQ ID NO: 14, ENA accession number M23197). SEQ ID NO: 14 is a wild-type gene sequence encoding CD33 protein, and is shown below:

(SEQ ID NO: 14)
GCTTCCTCAGACATGCCGCTGCTGCTACTGCTGCCCCTGCTGTGGGCAGGGGCCCTGGCT
ATGGATCCAAATTTCTGGCTGCAAGTGCAGGAGTCAGTGACGGTACAGGAGGGTTTGTGC
GTCCTCGTGCCCTGCACTTTCTTCCATCCCATACCCTACTACGACAAGAACTCCCCAGTT
CATGGTTACTGGTTCCGGGAAGGAGCCATTATATCCGGGGACTCTCCAGTGGCCACAAAC
AAGCTAGATCAAGAAGTACAGGAGGAGACTCAGGGCAGATTCCGCCTCCTTGGGGATCCC
AGTAGGAACAACTGCTCCCTGAGCATCGTAGACGCCAGGAGGAGGGATAATGGTTCATAC
TTCTTTCGGATGGAGAGAGGAAGTACCAAATACAGTTACAAATCTCCCCAGCTCTCTGTG
CATGTGACAGACTTGACCCACAGGCCCAAAATCCTCATCCCTGGCACTCTAGAACCCGGC
CACTCCAAAAACCTTACCTGCTCTGTGTCCTGGGCCTGTGAGCAGGGAACACCCCCGATC
TTCTCCTGGTTGTCAGCTGCCCCCACCTCCCTGGGCCCCAGGACTACTCACTCCTCGGTG
CTCATAATCACCCCACGGCCCCAGGACCACGGCACCAACCTGACCTGTCAGGTGAAGTTC
GCTGGAGCTGGTGTGACTACGGAGAGAACCATCCAGCTCAACGTCACCTATGTTCCACAG
AACCCAACAACTGGTATCTTTCCAGGAGATGGCTCAGGGAAACAAGAGACCAGAGCAGGA
CTGGTTCATGGGGCCATTGGAGGAGCTGGTGTTACAGCCCTGCTCGCTCTTTGTCTCTGC
CTCATCTTCTTCATAGTGAAGACCCACAGGAGGAAAGCAGCCAGGACAGCAGTGGGCAGC
AATGACACCCACCCTACCACAGGGTCAGCCTCCCCGAAACACCAGAAGAACTCCAAGTTA
CATGGCCCCACTGAAACCTCAAGCTGTTCAGGTGCCGCCCCTACTGTGGAGATGGATGAG
GAGCTGCATTATGCTTCCCTCAACTTTCATGGGATGAATCCTTCCAAGGACACCTCCACC
GAATACTCAGAGGTCAGGACCCAGTGAGGAACCCTCAAGAGCATCAGGCTCAGCTAGAAG
ATCCACATCCTCTACAGGTCGGGGACCAAAGGCTGATTCTTGGAGATTTAACTCCCCACA
GGCAATGGGTTTATAGACATTATGTGAGTTTCCTGCTATATTAACATCATCTTGAGACTT
TGCAAGCAGAGAGTCGTGGAATCAAATCTGTGCTCTTTCATTTGCTAAGTGTATGATGTC
ACACAAGCTCCTTAACCTTCCATGTCTCCATTTTCTTCTCTGTGAAGTAGGTATAAGAAG
TCCTATCTCATAGGGATGCTGTGAGCATTAAATAAAGGTACACATGGAAAACACCAG

As used herein, the term “CD68” refers to the gene encoding CD68 Molecule. The terms “CD68” and “CD68 molecule” include wild-type forms of the CD68 gene, as well as variants (e.g., splice variants and polymorphisms) of wild-type CD68. Examples of such variants are nucleic acids having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to a wild-type CD68 nucleic acid sequence (e.g., SEQ ID NO: 15, NCBI Reference Sequence: NM_001251.2). SEQ ID NO: 15 is a wild-type gene sequence encoding CD68 protein, and is shown below:

(SEQ ID NO: 15)
TTAATTACAAAAACTAATGACTAAGAGAGAGGTGGCTAGAGCTGAGGCCCCTGAGTCAGGCTGTGG
GTGGGATCATCTCCAGTACAGGAAGTGAGACTTTCATTTCCTCCTTTCCAAGAGAGGGCTGAGGGAG
CAGGGTTGAGCAACTGGTGCAGACAGCCTAGCTGGACTTTGGGTGAGGCGGTTCAGCCATGAGGCT
GGCTGTGCTTTTCTCGGGGGCCCTGCTGGGGCTACTGGCAGCCCAGGGGACAGGGAATGACTGTC
CTCACAAAAAATCAGCTACTTTGCTGCCATCCTTCACGGTGACACCCACGGTTACAGAGAGCACTGG
AACAACCAGCCACAGGACTACCAAGAGCCACAAAACCACCACTCACAGGACAACCACCACAGGCAC
CACCAGCCACGGACCCACGACTGCCACTCACAACCCCACCACCACCAGCCATGGAAACGTCACAGT
TCATCCAACAAGCAATAGCACTGCCACCAGCCAGGGACCCTCAACTGCCACTCACAGTCCTGCCAC
CACTAGTCATGGAAATGCCACGGTTCATCCAACAAGCAACAGCACTGCCACCAGCCCAGGATTCACC
AGTTCTGCCCACCCAGAACCACCTCCACCCTCTCCGAGTCCTAGCCCAACCTCCAAGGAGACCATT
GGAGACTACACGTGGACCAATGGTTCCCAGCCCTGTGTCCACCTCCAAGCCCAGATTCAGATTCGA
GTCATGTACACAACCCAGGGTGGAGGAGAGGCCTGGGGCATCTCTGTACTGAACCCCAACAAAACC
AAGGTCCAGGGAAGCTGTGAGGGTGCCCATCCCCACCTGCTTCTCTCATTCCCCTATGGACACCTC
AGCTTTGGATTCATGCAGGACCTCCAGCAGAAGGTTGTCTACCTGAGCTACATGGCGGTGGAGTAC
AATGTGTCCTTCCCCCACGCAGCACAGTGGACATTCTCGGCTCAGAATGCATCCCTTCGAGATCTCC
AAGCACCCCTGGGGCAGAGCTTCAGTTGCAGCAACTCGAGCATCATTCTTTCACCAGCTGTCCACCT
CGACCTGCTCTCCCTGAGGCTCCAGGCTGCTCAGCTGCCCCACACAGGGGTCTTTGGGCAAAGTTT
CTCCTGCCCCAGTGACCGGTCCATCTTGCTGCCTCTCATCATCGGCCTGATCCTTCTTGGCCTCCTC
GCCCTGGTGCTTATTGCTTTCTGCATCATCCGGAGACGCCCATCCGCCTACCAGGCCCTCTGAGCAT
TTGCTTCAAACCCCAGGGCACTGAGGGGGTTGGGGTGTGGTGGGGGGGTACCCTTATTTCCTCGAC
ACGCAACTGGCTCAAAGACAATGTTATTTTCCTTCCCTTTCTTGAAGAACAAAAAGAAAGCCGGGCAT
GACGGCTCATGCCTGTAATCCCAGCACTTTGGGAGGCTGAGGCAGGTGGATCACTGGAGGTCAGGA
GTTTGAGACCAGCCTGGCCAACATGGTGAAACCCTGTCTCTACTAAAAATACAATTAGCCAGGTGTG
GCGGCGTAATCCCAGCTGGCCTGTAATCCCAGCTACTTGGGAGGCTGAGGCAGAACTGCTTGAACC
CAGGAGGTGGAGGTTGCAGTGAGCCGTCATCGCGCCACTAAGCCAAGATCGCGCCACTGCACTCC
AGCCTGGGCGACAGAGCCAGACTGTCTCAAATAAATAAATATGAGATAATGCAGTCGGGAGAAGGG
AGGGAGAGAATTTTATTAAATGTGACGAACTGCCCCCCCCCCCCCCCCAGCAGGAGAGCAGCAAAA
TTTATGCAAATCTTTGACGGGGTTTTCCTTGTCCTGCCAGGATTAAAAGCCATGAGTTTCTTGTCAAA
AAAAAAAAAAAAAA

As used herein, the term “CLPTM1” refers to the gene encoding CLPTM1 Regulator of GABA Type A Receptor Forward Trafficking. The terms “CLPTM1” and “CLPTM1 Regulator of GABA Type A Receptor Forward Trafficking” include wild-type forms of the CLPTM1 gene, as well as variants (e.g., splice variants and polymorphisms) of wild-type CLPTM1. Examples of such variants are nucleic acids having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to a wild-type CLPTM1 nucleic acid sequence (e.g., SEQ ID NO: 16, NCBI Reference Sequence: NM_001294.3). SEQ ID NO: 16 is a wild-type gene sequence encoding CLPTM1 protein, and is shown below:

(SEQ ID NO: 16)
AGGTTGGTCCTTCCATAGCCGGAAGTGGCCTTCCTGAGAGGCGTGGCTGCGGCACTCTTGCCGGAT
AGGGTGGCCCGGCGGGGCTAGGAAAGCGTGAAATCTCGCGCGATTGCGCTGCGAAGTCGGGGAC
GGGGCGGGGCTGGCGGCGGGGGCGGGGACCCGGAGCGGGAAGATGGCGGCGGCGCAGGAGGC
GGACGGGGCCCGCAGCGCCGTGGTGGCGGCCGGGGGAGGCAGCTCCGGTCAGGTGACCAGCAAT
GGCAGCATCGGGAGGGACCCGCCAGCGGAGACCCAGCCTCAGAACCCACCGGCCCAGCCGGCAC
CCAATGCCTGGCAGGTCATCAAAGGTGTGCTGTTTAGGATCTTCATCATCTGGGCCATCAGCAGTTG
GTTCCGCCGAGGGCCGGCCCCTCAGGACCAGGCGGGCCCCGGAGGAGCTCCACGCGTCGCCAGC
CGCAACCTGTTCCCCAAAGACACTTTAATGAACCTGCATGTGTACATCTCAGAGCACGAGCACTTTA
CAGACTTCAACGCCACGTCGGCACTCTTCTGGGAACAGCACGATCTTGTGTATGGCGACTGGACTA
GCGGCGAGAACTCAGACGGCTGCTACGAGCACTTTGCTGAGCTCGATATCCCACAGAGCGTCCAGC
AGAACGGCTCCATCTACATCCACGTTTACTTCACCAAGAGTGGCTTCCACCCAGACCCCCGGCAGAA
GGCCCTGTACCGCCGGCTTGCCACAGTCCACATGTCCCGGATGATCAACAAATACAAGCGCAGACG
ATTTCAGAAAACCAAGAACCTGCTGACAGGAGAGACAGAAGCGGACCCAGAAATGATCAAGAGGGC
TGAGGACTATGGGCCTGTGGAGGTGATCTCCCATTGGCACCCCAACATCACCATCAACATCGTGGA
CGACCACACGCCGTGGGTGAAGGGCAGTGTGCCCCCTCCCCTGGATCAATATGTGAAGTTCGACGC
CGTGAGCGGTGACTACTATCCCATCATCTACTTCAATGACTACTGGAACCTGCAGAAGGACTACTAC
CCCATCAACGAGAGCCTGGCCAGCCTGCCGCTCCGCGTCTCCTTCTGCCCACTCTCGCTTTGGCGC
TGGCAGCTCTATGCTGCCCAGAGCACCAAGTCGCCCTGGAACTTCCTGGGTGATGAGTTGTACGAG
CAGTCAGATGAGGAGCAGGACTCGGTGAAGGTGGCCCTGCTGGAGACCAACCCCTACCTGCTGGC
GCTCACCATCATCGTGTCTATCGTTCACAGTGTCTTCGAGTTCCTGGCCTTCAAGAATGATATCCAGT
TCTGGAACAGCCGGCAGTCCCTGGAGGGCCTGTCCGTGCGCTCCGTCTTCTTCGGCGTTTTCCAGT
CATTCGTGGTCCTCCTCTACATCCTGGACAACGAGACCAACTTCGTGGTCCAGGTCAGCGTCTTCAT
TGGGGTCCTCATCGACCTCTGGAAGATCACCAAGGTCATGGACGTCCGGCTGGACCGAGAGCACAG
GGTGGCAGGAATCTTCCCCCGCCTATCCTTCAAGGACAAGTCCACGTATATCGAGTCCTCGACCAAA
GTGTATGATGATATGGCATTCCGGTACCTGTCCTGGATCCTCTTCCCGCTCCTGGGCTGCTATGCCG
TCTACAGTCTTCTGTACCTGGAGCACAAGGGCTGGTACTCCTGGGTGCTCAGCATGCTCTACGGCTT
CCTGCTGACCTTCGGCTTCATCACCATGACGCCCCAGCTCTTCATCAACTACAAGCTCAAGTCTGTG
GCCCACCTTCCCTGGCGCATGCTCACCTACAAGGCCCTCAACACATTCATCGACGACCTGTTCGCCT
TTGTCATCAAGATGCCCGTTATGTACCGGATCGGCTGCCTGCGGGACGATGTGGTTTTCTTCATCTA
CCTCTACCAACGGTGGATCTACCGCGTCGACCCCACCCGAGTCAACGAGTTTGGCATGAGTGGAGA
AGACCCCACAGCTGCCGCCCCCGTGGCCGAGGTTCCCACAGCAGCAGGGGCCCTCACGCCCACAC
CTGCACCCACCACGACCACCGCCACCAGGGAGGAGGCCTCCACGTCCCTGCCCACCAAGCCCACC
CAGGGGGCCAGCTCTGCCAGCGAGCCCCAGGAAGCCCCTCCAAAGCCAGCAGAGGACAAGAAAAA
GGATTAGTCGAGACTGGTCCTCACCTGCTCCGGCTCCTGGCGACCACTACCCCTGCGTCCCGGCCC
CCTCGCCTCCCCTCCCTGTCGCCCTTTCCCTGGACAGATCAGGCCGGGGCGGTGGGAGGCCCGCC
TCAGGTCAGGGCCCAGCGTGTGATGTAGGGGCCGGGGCAGGCCAGGGTTTGTTTGTGGAGGCGCT
GTCTGTCCCTCTGTCCCTCTGTGTTTCCAGCCATCTCGCCCTGCCAGCCCAGCACCACTGGGAATCA
TGGTGAAGCTGATGCAGCGTTGCCGAGGGGGGGGTTGGGGGGGGGGGGGCCGGGCCCCCCTA
CGGGATGCCCACGGCCGTTCATCATCTTGTCCCTCGTCCCCCTACCACACTCCCCCTCCTAGACCG
CCGCCCTTTAACACAGTCTGGATTTAATAAATTCATATGGGTGTTTAACTTAAACTCAGCACTAAAAAA
AAAAAAAAAAAA

As used herein, the term “CLU” refers to the gene encoding Clusterin. The terms “CLU” and “Clusterin” include wild-type forms of the CLU gene, as well as variants (e.g., splice variants and polymorphisms) of wild-type CLU. Examples of such variants are nucleic acids having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to a wild-type CLU nucleic acid sequence (e.g., SEQ ID NO: 17, ENA accession number M25915). SEQ ID NO: 17 is a wild-type gene sequence encoding CLU protein, and is shown below:

(SEQ ID NO: 17)
CTGCGAACCCTCTCTACTCTCCGAAGGGAATTGTCCTTCCTGGCTTCCACTACTTCCACC
CCTGAATGCACAGGCAGCCCGGCCCAAGTCTCCCACTAGGGATGCAGATGGATTCGGTGT
GAAGGGCTGGCTGCTGTTGCCTCCGGCTCTTGAAAGTCAAGTTCAGAGGCGTGCAAAGAC
TCCAGAATTGGAGGCATGATGAAGACTCTGCTGCTGTTTGTGGGGCTGCTGCTGACCTGG
GAGAGTGGGCAGGTCCTGGGGGACCAGACGGTCTCAGACAATGAGCTCCAGGAAATGTCC
AATCAGGGAAGTAAGTACGTCAATAAGGAAATTCAAAATGCTGTCAACGGGGTGAAACAG
ATAAAGACTCTCATAGAAAAAACAAACGAAGAGCGCAAGACACTGCTCAGCAACCTAGAA
GAAGCCAAGAAGAAGAAAGAGGATGCCCTAAATGAGACCAGGGAATCAGAGACAAAGCTG
AAGGAGCTCCCAGGAGTGTGCAATGAGACCATGATGGCCCTCTGGGAAGAGTGTAAGCCC
TGCCTGAAACAGACCTGCATGAAGTTCTACGCACGCGTCTGCAGAAGTGGCTCAGGCCTG
GTTGGCCGCCAGCTTGAGGAGTTCCTGAACCAGAGCTCGCCCTTCTACTTCTGGATGAAT
GGTGACCGCATCGACTCCCTGCTGGAGAACGACCGGCAGCAGACGCACATGCTGGATGTC
ATGCAGGACCACTTCAGCCGCGCGTCCAGCATCATAGACGAGCTCTTCCAGGACAGGTTC
TTCACCCGGGAGCCCCAGGATACCTACCACTACCTGCCCTTCAGCCTGCCCCACCGGAGG
CCTCACTTCTTCTTTCCCAAGTCCCGCATCGTCCGCAGCTTGATGCCCTTCTCTCCGTAC
GAGCCCCTGAACTTCCACGCCATGTTCCAGCCCTTCCTTGAGATGATACACGAGGCTCAG
CAGGCCATGGACATCCACTTCCACAGCCCGGCCTTCCAGCACCCGCCAACAGAATTCATA
CGAGAAGGCGACGATGACCGGACTGTGTGCCGGGAGATCCGCCACAACTCCACGGGCTGC
CTGCGGATGAAGGACCAGTGTGACAAGTGCCGGGAGATCTTGTCTGTGGACTGTTCCACC
AACAACCCCTCCCAGGCTAAGCTGCGGCGGGAGCTCGACGAATCCCTCCAGGTCGCTGAG
AGGTTGACCAGGAAATATAACGAGCTGCTAAAGTCCTACCAGTGGAAGATGCTCAACACC
TCCTCCTTGCTGGAGCAGCTGAACGAGCAGTTTAACTGGGTGTCCCGGCTGGCAAACCTC
ACGCAAGGCGAAGACCAGTACTATCTGCGGGTCACCACGGTGGCTTCCCACACTTCTGAC
TCGGACGTTCCTTCCGGTGTCACTGAGGTGGTCGTGAAGCTCTTTGACTCTGATCCCATC
ACTGTGACGGTCCCTGTAGAAGTCTCCAGGAAGAACCCTAAATTTATGGAGACCGTGGCG
GAGAAAGCGCTGCAGGAATACCGCAAAAAGCACCGGGAGGAGTGAGATGTGGATGTTGCT
TTTGCACCTACGGGGGCATCTGAGTCCAGCTCCCCCCAAGATGAGCTGCAGCCCCCCAGA
GAGAGCTCTGCACGTCACCAAGTAACCAGGC

As used herein, the term “CR1” refers to the gene encoding Complement receptor type 1. The terms “CR1” and “Complement receptor type 1” include wild-type forms of the CR1 gene, as well as variants (e.g., splice variants and polymorphisms) of wild-type CR1. Examples of such variants are nucleic acids having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to a wild-type CR1 nucleic acid sequence (e.g., SEQ ID NO: 18, ENA accession number Y00816). SEQ ID NO: 18 is a wild-type gene sequence encoding CR1 protein, and is shown below:

(SEQ ID NO: 18)
CGTGGTTTGTAGATGTGCTTGGGGAGAATGGGGGCCTCTTCTCCAAGAAGCCCGGAGCCT
GTCGGGCCGCCGGCGCCCGGTCTCCCCTTCTGCTGCGGAGGATCCCTGCTGGCGGTTGTG
GTGCTGCTTGCGCTGCCGGTGGCCTGGGGTCAATGCAATGCCCCAGAATGGCTTCCATTT
GCCAGGCCTACCAACCTAACTGATGAGTTTGAGTTTCCCATTGGGACATATCTGAACTAT
GAATGCCGCCCTGGTTATTCCGGAAGACCGTTTTCTATCATCTGCCTAAAAAACTCAGTC
TGGACTGGTGCTAAGGACAGGTGCAGACGTAAATCATGTCGTAATCCTCCAGATCCTGTG
AATGGCATGGTGCATGTGATCAAAGGCATCCAGTTCGGATCCCAAATTAAATATTCTTGT
ACTAAAGGATACCGACTCATTGGTTCCTCGTCTGCCACATGCATCATCTCAGGTGATACT
GTCATTTGGGATAATGAAACACCTATTTGTGACAGAATTCCTTGTGGGCTACCCCCCACC
ATCACCAATGGAGATTTCATTAGCACCAACAGAGAGAATTTTCACTATGGATCAGTGGTG
ACCTACCGCTGCAATCCTGGAAGCGGAGGGAGAAAGGTGTTTGAGCTTGTGGGTGAGCCC
TCCATATACTGCACCAGCAATGACGATCAAGTGGGCATCTGGAGCGGCCCCGCCCCTCAG
TGCATTATACCTAACAAATGCACGCCTCCAAATGTGGAAAATGGAATATTGGTATCTGAC
AACAGAAGCTTATTTTCCTTAAATGAAGTTGTGGAGTTTAGGTGTCAGCCTGGCTTTGTC
ATGAAAGGACCCCGCCGTGTGAAGTGCCAGGCCCTGAACAAATGGGAGCCGGAGCTACCA
AGCTGCTCCAGGGTATGTCAGCCACCTCCAGATGTCCTGCATGCTGAGCGTACCCAAAGG
GACAAGGACAACTTTTCACCTGGGCAGGAAGTGTTCTACAGCTGTGAGCCCGGCTACGAC
CTCAGAGGGGCTGCGTCTATGCGCTGCACACCCCAGGGAGACTGGAGCCCTGCAGCCCCC
ACATGTGAAGTGAAATCCTGTGATGACTTCATGGGCCAACTTCTTAATGGCCGTGTGCTA
TTTCCAGTAAATCTCCAGCTTGGAGCAAAAGTGGATTTTGTTTGTGATGAAGGATTTCAA
TTAAAAGGCAGCTCTGCTAGTTACTGTGTCTTGGCTGGAATGGAAAGCCTTTGGAATAGC
AGTGTTCCAGTGTGTGAACAAATCTTTTGTCCAAGTCCTCCAGTTATTCCTAATGGGAGA
CACACAGGAAAACCTCTGGAAGTCTTTCCCTTTGGAAAAGCAGTAAATTACACATGCGAC
CCCCACCCAGACAGAGGGACGAGCTTCGACCTCATTGGAGAGAGCACCATCCGCTGCACA
AGTGACCCTCAAGGGAATGGGGTTTGGAGCAGCCCTGCCCCTCGCTGTGGAATTCTGGGT
CACTGTCAAGCCCCAGATCATTTTCTGTTTGCCAAGTTGAAAACCCAAACCAATGCATCT
GACTTTCCCATTGGGACATCTTTAAAGTACGAATGCCGTCCTGAGTACTACGGGAGGCCA
TTCTCTATCACATGTCTAGATAACCTGGTCTGGTCAAGTCCCAAAGATGTCTGTAAACGT
AAATCATGTAAAACTCCTCCAGATCCAGTGAATGGCATGGTGCATGTGATCACAGACATC
CAGGTTGGATCCAGAATCAACTATTCTTGTACTACAGGGCACCGACTCATTGGTCACTCA
TCTGCTGAATGTATCCTCTCGGGCAATGCTGCCCATTGGAGCACGAAGCCGCCAATTTGT
CAACGAATTCCTTGTGGGCTACCCCCCACCATCGCCAATGGAGATTTCATTAGCACCAAC
AGAGAGAATTTTCACTATGGATCAGTGGTGACCTACCGCTGCAATCCTGGAAGCGGAGGG
AGAAAGGTGTTTGAGCTTGTGGGTGAGCCCTCCATATACTGCACCAGCAATGACGATCAA
GTGGGCATCTGGAGCGGCCCGGCCCCTCAGTGCATTATACCTAACAAATGCACGCCTCCA
AATGTGGAAAATGGAATATTGGTATCTGACAACAGAAGCTTATTTTCCTTAAATGAAGTT
GTGGAGTTTAGGTGTCAGCCTGGCTTTGTCATGAAAGGACCCCGCCGTGTGAAGTGCCAG
GCCCTGAACAAATGGGAGCCGGAGCTACCAAGCTGCTCCAGGGTATGTCAGCCACCTCCA
GATGTCCTGCATGCTGAGCGTACCCAAAGGGACAAGGACAACTTTTCACCCGGGCAGGAA
GTGTTCTACAGCTGTGAGCCCGGCTATGACCTCAGAGGGGCTGCGTCTATGCGCTGCACA
CCCCAGGGAGACTGGAGCCCTGCAGCCCCCACATGTGAAGTGAAATCCTGTGATGACTTC
ATGGGCCAACTTCTTAATGGCCGTGTGCTATTTCCAGTAAATCTCCAGCTTGGAGCAAAA
GTGGATTTTGTTTGTGATGAAGGATTTCAATTAAAAGGCAGCTCTGCTAGTTATTGTGTC
TTGGCTGGAATGGAAAGCCTTTGGAATAGCAGTGTTCCAGTGTGTGAACAAATCTTTTGT
CCAAGTCCTCCAGTTATTCCTAATGGGAGACACACAGGAAAACCTCTGGAAGTCTTTCCC
TTTGGAAAAGCAGTAAATTACACATGCGACCCCCACCCAGACAGAGGGACGAGCTTCGAC
CTCATTGGAGAGAGCACCATCCGCTGCACAAGTGACCCTCAAGGGAATGGGGTTTGGAGC
AGCCCTGCCCCTCGCTGTGGAATTCTGGGTCACTGTCAAGCCCCAGATCATTTTCTGTTT
GCCAAGTTGAAAACCCAAACCAATGCATCTGACTTTCCCATTGGGACATCTTTAAAGTAC
GAATGCCGTCCTGAGTACTACGGGAGGCCATTCTCTATCACATGTCTAGATAACCTGGTC
TGGTCAAGTCCCAAAGATGTCTGTAAACGTAAATCATGTAAAACTCCTCCAGATCCAGTG
AATGGCATGGTGCATGTGATCACAGACATCCAGGTTGGATCCAGAATCAACTATTCTTGT
ACTACAGGGCACCGACTCATTGGTCACTCATCTGCTGAATGTATCCTCTCAGGCAATACT
GCCCATTGGAGCACGAAGCCGCCAATTTGTCAACGAATTCCTTGTGGGCTACCCCCAACC
ATCGCCAATGGAGATTTCATTAGCACCAACAGAGAGAATTTTCACTATGGATCAGTGGTG
ACCTACCGCTGCAATCTTGGAAGCAGAGGGAGAAAGGTGTTTGAGCTTGTGGGTGAGCCC
TCCATATACTGCACCAGCAATGACGATCAAGTGGGCATCTGGAGCGGCCCCGCCCCTCAG
TGCATTATACCTAACAAATGCACGCCTCCAAATGTGGAAAATGGAATATTGGTATCTGAC
AACAGAAGCTTATTTTCCTTAAATGAAGTTGTGGAGTTTAGGTGTCAGCCTGGCTTTGTC
ATGAAAGGACCCCGCCGTGTGAAGTGCCAGGCCCTGAACAAATGGGAGCCAGAGTTACCA
AGCTGCTCCAGGGTGTGTCAGCCGCCTCCAGAAATCCTGCATGGTGAGCATACCCCAAGC
CATCAGGACAACTTTTCACCTGGGCAGGAAGTGTTCTACAGCTGTGAGCCTGGCTATGAC
CTCAGAGGGGCTGCGTCTCTGCACTGCACACCCCAGGGAGACTGGAGCCCTGAAGCCCCG
AGATGTGCAGTGAAATCCTGTGATGACTTCTTGGGTCAACTCCCTCATGGCCGTGTGCTA
TTTCCACTTAATCTCCAGCTTGGGGCAAAGGTGTCCTTTGTCTGTGATGAAGGGTTTCGC
TTAAAGGGCAGTTCCGTTAGTCATTGTGTCTTGGTTGGAATGAGAAGCCTTTGGAATAAC
AGTGTTCCTGTGTGTGAACATATCTTTTGTCCAAATCCTCCAGCTATCCTTAATGGGAGA
CACACAGGAACTCCCTCTGGAGATATTCCCTATGGAAAAGAAATATCTTACACATGTGAC
CCCCACCCAGACAGAGGGATGACCTTCAACCTCATTGGGGAGAGCACCATCCGCTGCACA
AGTGACCCTCATGGGAATGGGGTTTGGAGCAGCCCTGCCCCTCGCTGTGAACTTTCTGTT
CGTGCTGGTCACTGTAAAACCCCAGAGCAGTTTCCATTTGCCAGTCCTACGATCCCAATT
AATGACTTTGAGTTTCCAGTCGGGACATCTTTGAATTATGAATGCCGTCCTGGGTATTTT
GGGAAAATGTTCTCTATCTCCTGCCTAGAAAACTTGGTCTGGTCAAGTGTTGAAGACAAC
TGTAGACGAAAATCATGTGGACCTCCACCAGAACCCTTCAATGGAATGGTGCATATAAAC
ACAGATACACAGTTTGGATCAACAGTTAATTATTCTTGTAATGAAGGGTTTCGACTCATT
GGTTCCCCATCTACTACTTGTCTCGTCTCAGGCAATAATGTCACATGGGATAAGAAGGCA
CCTATTTGTGAGATCATATCTTGTGAGCCACCTCCAACCATATCCAATGGAGACTTCTAC
AGCAACAATAGAACATCTTTTCACAATGGAACGGTGGTAACTTACCAGTGCCACACTGGA
CCAGATGGAGAACAGCTGTTTGAGCTTGTGGGAGAACGGTCAATATATTGCACCAGCAAA
GATGATCAAGTTGGTGTTTGGAGCAGCCCTCCCCCTCGGTGTATTTCTACTAATAAATGC
ACAGCTCCAGAAGTTGAAAATGCAATTAGAGTACCAGGAAACAGGAGTTTCTTTTCCCTC
ACTGAGATCATCAGATTTAGATGTCAGCCCGGGTTTGTCATGGTAGGGTCCCACACTGTG
CAGTGCCAGACCAATGGCAGATGGGGGCCCAAGCTGCCACACTGCTCCAGGGTGTGTCAG
CCGCCTCCAGAAATCCTGCATGGTGAGCATACCCTAAGCCATCAGGACAACTTTTCACCT
GGGCAGGAAGTGTTCTACAGCTGTGAGCCCAGCTATGACCTCAGAGGGGCTGCGTCTCTG
CACTGCACGCCCCAGGGAGACTGGAGCCCTGAAGCCCCTAGATGTACAGTGAAATCCTGT
GATGACTTCCTGGGCCAACTCCCTCATGGCCGTGTGCTACTTCCACTTAATCTCCAGCTT
GGGGCAAAGGTGTCCTTTGTTTGCGATGAAGGGTTCCGATTAAAAGGCAGGTCTGCTAGT
CATTGTGTCTTGGCTGGAATGAAAGCCCTTTGGAATAGCAGTGTTCCAGTGTGTGAACAA
ATCTTTTGTCCAAATCCTCCAGCTATCCTTAATGGGAGACACACAGGAACTCCCTTTGGA
GATATTCCCTATGGAAAAGAAATATCTTACGCATGCGACACCCACCCAGACAGAGGGATG
ACCTTCAACCTCATTGGGGAGAGCTCCATCCGCTGCACAAGTGACCCTCAAGGGAATGGG
GTTTGGAGCAGCCCTGCCCCTCGCTGTGAACTTTCTGTTCCTGCTGCCTGCCCACATCCA
CCCAAGATCCAAAACGGGCATTACATTGGAGGACACGTATCTCTATATCTTCCTGGGATG
ACAATCAGCTACACTTGTGACCCCGGCTACCTGTTAGTGGGAAAGGGCTTCATTTTCTGT
ACAGACCAGGGAATCTGGAGCCAATTGGATCATTATTGCAAAGAAGTAAATTGTAGCTTC
CCACTGTTTATGAATGGAATCTCGAAGGAGTTAGAAATGAAAAAAGTATATCACTATGGA
GATTATGTGACTTTGAAGTGTGAAGATGGGTATACTCTGGAAGGCAGTCCCTGGAGCCAG
TGCCAGGCGGATGACAGATGGGACCCTCCTCTGGCCAAATGTACCTCTCGTGCACATGAT
GCTCTCATAGTTGGCACTTTATCTGGTACGATCTTCTTTATTTTACTCATCATTTTCCTC
TCTTGGATAATTCTAAAGCACAGAAAAGGCAATAATGCACATGAAAACCCTAAAGAAGTG
GCTATCCATTTACATTCTCAAGGAGGCAGCAGCGTTCATCCCCGAACTCTGCAAACAAAT
GAAGAAAATAGCAGGGTCCTTCCTTGACAAAGTACTATACAGCTGAAGAACATCTCGAAT
ACAATTTTGGTGGGAAAGGAGCCAATTGATTTCAACAGAATCAGATCTGAGCTTCATAAA
GTCTTTGAAGTGACTTCACAGAGACGCAGACATGTGCACTTGAAGATGCTGCCCCTTCCC
TGGTACCTAGCAAAGCTCCTGCCTCTTTGTGTGCGTCACTGTGAAACCCCCACCCTTCTG
CCTCGTGCTAAACGCACACAGTATCTAGTCAGGGGAAAAGACTGCATTTAGGAGATAGAA
AATAGTTTGGATTACTTAAAGGAATAAGGTGTTGCCTGGAATTTCTGGTTTGTAAGGTGG
TCACTGTTCTTTTTTAAAATATTTGTAATATGGAATGGGCTCAGTAAGAAGAGCTTGGAA
AATGCAGAAAGTTATGAAAAATAAGTCACTTATAATTATGCTACCTACTGATAACCACTC
CTAATATTTTGATTCATTTTCTGCCTATCTTCTTTCACATATGTGTTTTTTTACATACGT
ACTTTTCCCCCCTTAGTTTGTTTCCTTTTATTTTATAGAGCAGAACCCTAGTCTTTTAAA
CAGTTTAGAGTGAAATATATGCTATATCAGTTTTTACTTTCTCTAGGGAGAAAAATTAAT
TTACTAGAAAGGCATGAAATGATCATGGGAAGAGTGGTTAAGACTACTGAAGAGAAATAT
TTGGAAAATAAGATTTCGATATCTTCTTTTTTTTTGAGATGGAGTCTGGCTCTGTCTCCC
AGGCTGGAGTGCAGTGGCGTAATCTCGGCTCACTGCAACGTCCGCCTCCCG

As used herein, the term “CSF1” refers to the gene encoding Macrophage colony-stimulating factor 1. The terms “CSF1” and “Macrophage colony-stimulating factor 1” include wild-type forms of the CSF1 gene, as well as variants (e.g., splice variants and polymorphisms) of wild-type CSF1. Examples of such variants are nucleic acids having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to a wild-type CSF1 nucleic acid sequence (e.g., SEQ ID NO: 19, ENA accession number M37435). SEQ ID NO: 19 is a wild-type gene sequence encoding CSF1 protein, and is shown below:

(SEQ ID NO: 19)
CCTGGGTCCTCTCGGCGCCAGAGCCGCTCTCCGCATCCCAGGACAGCGGTGCGGCCCTCG
GCCGGGGCGCCCACTCCGCAGCAGCCAGCGAGCCAGCTGCCCCGTATGACCGCGCCGGGC
GCCGCCGGGCGCTGCCCTCCCACGACATGGCTGGGCTCCCTGCTGTTGTTGGTCTGTCTC
CTGGCGAGCAGGAGTATCACCGAGGAGGTGTCGGAGTACTGTAGCCACATGATTGGGAGT
GGACACCTGCAGTCTCTGCAGCGGCTGATTGACAGTCAGATGGAGACCTCGTGCCAAATT
ACATTTGAGTTTGTAGACCAGGAACAGTTGAAAGATCCAGTGTGCTACCTTAAGAAGGCA
TTTCTCCTGGTACAAGACATAATGGAGGACACCATGCGCTTCAGAGATAACACCGCCAAT
CCCATCGCCATTGTGCAGCTGCAGGAACTCTCTTTGAGGCTGAAGAGCTGCTTCACCAAG
GATTATGAAGAGCATGACAAGGCCTGCGTCCGAACTTTCTATGAGACACCTCTCCAGTTG
CTGGAGAAGGTCAAGAATGTCTTTAATGAAACAAAGAATCTCCTTGACAAGGACTGGAAT
ATTTTCAGCAAGAACTGCAACAACAGCTTTGCTGAATGCTCCAGCCAAGATGTGGTGACC
AAGCCTGATTGCAACTGCCTGTACCCCAAAGCCATCCCTAGCAGTGACCCGGCCTCTGTC
TCCCCTCATCAGCCCCTCGCCCCCTCCATGGCCCCTGTGGCTGGCTTGACCTGGGAGGAC
TCTGAGGGAACTGAGGGCAGCTCCCTCTTGCCTGGTGAGCAGCCCCTGCACACAGTGGAT
CCAGGCAGTGCCAAGCAGCGGCCACCCAGGAGCACCTGCCAGAGCTTTGAGCCGCCAGAG
ACCCCAGTTGTCAAGGACAGCACCATCGGTGGCTCACCACAGCCTCGCCCCTCTGTCGGG
GCCTTCAACCCCGGGATGGAGGATATTCTTGACTCTGCAATGGGCACTAATTGGGTCCCA
GAAGAAGCCTCTGGAGAGGCCAGTGAGATTCCCGTACCCCAAGGGACAGAGCTTTCCCCC
TCCAGGCCAGGAGGGGGCAGCATGCAGACAGAGCCCGCCAGACCCAGCAACTTCCTCTCA
GCATCTTCTCCACTCCCTGCATCAGCAAAGGGCCAACAGCCGGCAGATGTAACTGCTACA
GCCTTGCCCAGGGTGGGCCCCGTGATGCCCACTGGCCAGGACTGGAATCACACCCCCCAG
AAGACAGACCATCCATCTGCCCTGCTCAGAGACCCCCCGGAGCCAGGCTCTCCCAGGATC
TCATCACTGCGCCCCCAGGCCCTCAGCAACCCCTCCACCCTCTCTGCTCAGCCACAGCTT
TCCAGAAGCCACTCCTCGGGCAGCGTGCTGCCCCTTGGGGAGCTGGAGGGCAGGAGGAGC
ACCAGGGATCGGACGAGCCCCGCAGAGCCAGAAGCAGCACCAGCAAGTGAAGGGGCAGCC
AGGCCCCTGCCCCGTTTTAACTCCGTTCCTTTGACTGACACAGGCCATGAGAGGCAGTCC
GAGGGATCCTCCAGCCCGCAGCTCCAGGAGTCTGTCTTCCACCTGCTGGTGCCCAGTGTC
ATCCTGGTCTTGCTGGCTGTCGGAGGCCTCTTGTTCTACAGGTGGAGGCGGCGGAGCCAT
CAAGAGCCTCAGAGAGCGGATTCTCCCTTGGAGCAACCAGAGGGCAGCCCCCTGACTCAG
GATGACAGACAGGTGGAACTGCCAGTGTAGAGGGAATTCTAAGCTGGACGCACAGAACAG
TCTCTTCGTGGGAGGAGACATTATGGGGCGTCCACCACCACCCCTCCCTGGCCATCCTCC
TGGAATGTGGTCTGCCCTCCACCAGAGCTCCTGCCTGCCAGGACTGGACCAGAGCAGCCA
GGCTGGGGCCCCTCTGTCTCAACCCGCAGACCCTTGACTGAATGAGAGAGGCCAGAGGAT
GCTCCCCATGCTGCCACTATTTATTGTGAGCCCTGGAGGCTCCCATGTGCTTGAGGAAGG
CTGGTGAGCCCGGCTCAGGACCCTCTTCCCTCAGGGGCTGCAGCCTCCTCTCACTCCCTT
CCATGCCGGAACCCAGGCCAGGGACCCACCGGCCTGTGGTTTGTGGGAAAGCAGGGTGCA
CGCTGAGGAGTGAAACAACCCTGCACCCAGAGGGCCTGCCTGGTGCCAAGGTATCCCAGC
CTGGACAGGCATGGACCTGTCTCCAGACAGAGGAGCCTGAAGTTCGTGGGGGGGGACAGC
CTCGGCCTGATTTCCCGTAAAGGTGTGCAGCCTGAGAGACGGGAAGAGGAGGCCTCTGCA
CCTGCTGGTCTGCACTGACAGCCTGAAGGGTCTACACCCTCGGCTCACCTAAGTCCCTGT
GCTGGTTGCCAGGCCCAGAGGGGAGGCCAGCCCTGCCCTCAGGACCTGCCTGACCTGCCA
GTGATGCCAAGAGGGGGATCAAGCACTGGCCTCTGCCCCTCCTCCTTCCAGCACCTGCCA
GAGCTTCTCCAGCAGGCCAAGCAGAGGCTCCCCTCATGAAGGAAGCCATTGCACTGTGAA
CACTGTACCTGCCTGCTGAACAGCCTCCCCCCGTCCATCCATGAGCCAGCATCCGTCCGT
CCTCCACTCTCCAGCCTCTCCCCAGCCTCCTGCACTGAGCTGGCCTCACCAGTCGACTGA
GGGAGCCCCTCAGCCCTGACCTTCTCCTGACCTGGCCTTTGACTCCCCGGAGTGGAGTGG
GGTGGGAGAACCTCCTGGGCCGCCAGCCAGAGCCGCTCTTTAGGCTGTGTTCTTCGCCCA
GGTTTCTGCATCTTCCACTTTGACATTCCCAAGAGGGAAGGGACTAGTGGGAGAGAGCAA
GGGAGGGGAGGGCACAGACAGAGAGCCTACAGGGCGAGCTCTGACTGAAGATGGGCCTTT
GAAATATAGGTATGCACCTGAGGTTGGGGGAGGGTCTGCACTCCCAAACCCCAGCGCAGT
GTCCTTTCCCTGCTGCCGACAGGAACCTGGGGCTGAGCAGGTTATCCCTGTCAGGAGCCC
TGGACTGGGCTGCATCTCAGCCCCACCTGCATGGTATCCAGCTCCCATCCACTTCTCACC
CTTCTTTCCTCCTGACCTTGGTCAGCAGTGATGACCTCCAACTCTCACCCACCCCCTCTA
CCATCACCTCTAACCAGGCAAGCCAGGGTGGGAGAGCAATCAGGAGAGCCAGGCCTCAGC
TTCCAATGCCTGGAGGGCCTCCACTTTGTGGCCAGCCTGTGGTGCTGGCTCTGAGGCCTA
GGCAACGAGCGACAGGGCTGCCAGTTGCCCCTGGGTTCCTTTGTGCTGCTGTGTGCCTCC
TCTCCTGCCGCCCTTTGTCCTCCGCTAAGAGACCCTGCCCTACCTGGCCGCTGGGCCCCG
TGACTTTCCCTTCCTGCCCAGGAAAGTGAGGGTCGGCTGGCCCCACCTTCCCTGTCCTGA
TGCCGACAGCTTAGGGAAGGGCACTGAACTTGCATATGGGGCTTAGCCTTCTAGTCACAG
CCTCTATATTTGATGCTAGAAAACACATATTTTTAAATGGAAGAAAAATAAAAAGGCATT
CCCCCTTCATCCCCCTACCTTAAACATATAATATTTTAAAGGTCAAAAAAGCAATCCAAC
CCACTGCAGAAGCTCTTTTTGAGCACTTGGTGGCATCAGAGCAGGAGGAGCCCCAGAGCC
ACCTCTGGTGTCCCCCAGGCTACCTGCTCAGGAACCCCTTCTGTTCTCTGAGAACTCAAC
AGAGGACATTGGCTCACGCACTGTGAGATTTTGTTTTTATACTTGCAACTGGTGAATTAT
TTTTTATAAAGTCATTTAAATATCTATTTAAAAGATAGGAAGCTGCTTATATATTTAATA
ATAAAAGAAGTGCACAAGCTGCCGTTGACGTAGCTCGAG

As used herein, the term “CST7” refers to the gene encoding Cystatin-F. The terms “CST7” and “Cystatin-F” include wild-type forms of the CST7 gene, as well as variants (e.g., splice variants and polymorphisms) of wild-type CST7. Examples of such variants are nucleic acids having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to a wild-type CST7 nucleic acid sequence (e.g., SEQ ID NO: 20, ENA accession number AF031824). SEQ ID NO: 20 is a wild-type gene sequence encoding CST7 protein, and is shown below:

(SEQ ID NO: 20)
GGCTCAGCACAGGCACAAACCATTGCCCGGCACTGGCCCGTGCTGCCTGAGAAGGATTGG
CACGGGCACAGACCACTGCCCCCACCTGCCCTGCGCCATCTACCCAAGAAGGCTCGGCAC
GGGCACCAACCACTGCCTCCAACTGCCCCATGCTGCCTGAGAAGGCACTGCACGGCCACC
CCCAACTGCCCCGCACTGTCCCTACCCGGGCAGCCATGCGAGCGGCTGGAACTCTGCTGG
CCTTCTGCTGCCTGGTCTTGAGCACCACTGGGGGCCCTTCCCCAGATACTTGTTCCCAGG
ACCTTAACTCACGTGTGAAGCCAGGATTTCCTAAAACAATAAAGACCAATGACCCAGGAG
TCCTCCAAGCAGCCAGATACAGTGTTGAAAAGTTCAACAACTGCACGAACGACATGTTCT
TGTTCAAGGAGTCCCGCATCACAAGGGCCCTAGTTCAGATAGTGAAAGGCCTGAAATATA
TGCTGGAGGTGGAAATTGGCAGAACTACCTGCAAGAAAAACCAGCACCTGCGTCTGGATG
ACTGTGACTTCCAAACCAACCACACCTTGAAGCAGACTCTGAGCTGCTACTCTGAAGTCT
GGGTCGTGCCCTGGCTCCAGCACTTCGAGGTGCCTGTTCTCCGTTGTCACTGACCCCCGC
CTCTTCAGCAAGACCACAGCCATGACAAACACCAGGATGCATGCTCCTTGTCCCCTCCCA
CCCGCCTCATGACCCAGCCTCACAGACCCTCTCAGGCCTCTGACGAGTGAGCGGGTGAAG
TGCCACTGGGTCACCGCAGGGCAGCTGGAATGGCAGCATGGTAGCACCTCCTAACAGATT
AAATAGATCACATTTGCTTCTAAAATT

As used herein, the term “CTSB” refers to the gene encoding Cathepsin B. The terms “CTSB” and “Cathepsin B” include wild-type forms of the CTSB gene, as well as variants (e.g., splice variants and polymorphisms) of wild-type CTSB. Examples of such variants are nucleic acids having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to a wild-type CTSB nucleic acid sequence (e.g., SEQ ID NO: 21, ENA accession number M14221). SEQ ID NO: 21 is a wild-type gene sequence encoding CTSB protein, and is shown below:

(SEQ ID NO: 21)
AATTCCGCGGCAACCGCTCCGGCAACGCCAACCGCTCCGCTGCGCGCAGGCTGGGCTGCA
GGCTCTCGGCTGCAGCGCTGGGCTGGTGTGCAGTGGTGCGACCACGGCTCACGGCAGCCT
CAGCCACCCAGATGTAAGCGATCTGGTTCCCACCTCAGCCTTCCGAGTAGTGGATCTAGG
ATCTGGCTTCCAACATGTGGCAGCTCTGGGCCTCCCTCTGCTGCCTGCTGGTGTTGGCCA
ATGCCCGGAGCAGGCCCTCTTTCCATCCCGTGTOGGATGAGCTGGTCAACTATGTCAACA
AACGGAATACCACGTGGCAGGCCGGGCACAACTTCTACAACGTGGACATGAGCTACTTGA
AGAGGCTATGTGGTACCTTCCTGGGTGGGCCCAAGCCACCCCAGAGAGTTATGTTTACCG
AGGACCTGAAGCTGCCTGCAAGCTTCGATGCACGGGAACAATGGCCACAGTGTCCCACCA
TCAAAGAGATCAGAGACCAGGGCTCCTGTGGCTCCTGCTGGGCCTTCGGGGCTGTGGAAG
CCATCTCTGACCGCATCTGCATCCACACCAATGCGCACGTCAGCGTGGAGGTGTCGGCGG
AGGACCTGCTCACCTGCTGTGGCAGCATGTGTGGGGACGGCTGTAATGGTGGCTATCCTG
CTGAAGCTTGGAACTTCTGGACAAGAAAAGGCCTGGTTTCTGGTGGCCTCTATGAATCCC
ATGTAGGGTGCAGACCGTACTCCATCCCTCCCTGTGAGCACCACGTCAACGGCTCCCGGC
CCCCATGCACGGGGGAGGGAGATACCCCCAAGTGTAGCAAGATCTGTGAGCCTGGCTACA
GCCCGACCTACAAACAGGACAAGCACTACGGATACAATTCCTACAGCGTCTCCAATAGCG
AGAAGGACATCATGGCCGAGATCTACAAAAACGGCCCCGTGGAGGGAGCTTTCTCTGTGT
ATTCGGACTTCCTGCTCTACAAGTCAGGAGTGTACCAACACGTCACCGGAGAGATGATGG
GTGGCCATGCCATCCGCATCCTGGGCTGGGGAGTGGAGAATGGCACACCCTACTGGCTGG
TTGCCAACTCCTGGAACACTGACTGGGGTGACAATGGCTTCTTTAAAATACTCAGAGGAC
AGGATCACTGCGGAATCGAATCAGAAGTGGTGGCTGGAATTCCACGCACCGATCAGTACT
GGGAAAAGATCTAATCTGCCGTGGGCCTGTCGTGCCAGTCCTGGGGGCGAGATCGGGGTA
GAAAGTCATTTTATTCTTTAAGTTCACGTAAGATACAAGTTTCAGGCAGGGTCTGAAGGA
CTGGATTGGCCAAAGTCCTCCAAGGAGACCAAGTCCTGGCTACATCCCAGCCTGTGGTTA
CAGTGCAGACAGGCCATGTGAGCCACCGCTGCCAGCACAGAGCGTCCTTCCCCCTGTAGA
CTAGTGCCGTGGGAGTACCTGCTGCCCAGCTGCTGTGGCCCCCTCCGTGATCCATCCATC
TCCAGGGAGCAAGACAGAGACGCAGGATGGAAAGCGGAGTTCCTAACAGGATGAAAGTTC
CCCCATCAGTTCCCCCAGTACCTCCAAGCAAGTAGCTTTCCACATTTGTCACAGAAATCA
GAGGAGAGATGGTGTTGGGAGCCCTTTGGAGAACGCCAGTCTCCAGGTCCCCCTGCATCT
ATCGAGTTTGCAATGTCACAACCTCTCTGATCTTGTGCTCAGCATGATTCTTTAATAGAA
GTTTTATTTTTCGTGCACTCTGCTAATCATGTGGGTGAGCCAGTGGAACAGCGGGAGCCT
GTGCTGGTTTGCAGATTGCCTCCTAATGACGCGGCTCAAAAGGAAACCAAGTGGTCAGGA
GTTGTTTCTGACCCACTGATCTCTACTACCACAAGGAAAATAGTTTAGGAGAAACCAGCT
TTTACTGTTTTTGAAAAATTACAGCTTCACCCTGTCAAGTTAACAAGGAATGCCTGTGCC
AATAAAAGGTTTCTCCAACTTG

As used herein, the term “CTSD” refers to the gene encoding Cathepsin D. The terms “CTSD” and “Cathepsin D” include wild-type forms of the CTSD gene, as well as variants (e.g., splice variants and polymorphisms) of wild-type CTSD. Examples of such variants are nucleic acids having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to a wild-type CTSD nucleic acid sequence (e.g., SEQ ID NO: 22, ENA accession number M11233). SEQ ID NO: 22 is a wild-type gene sequence encoding CTSD protein, and is shown below:

(SEQ ID NO: 22)
GGCTATAAGCGCACGGCCTCGGCGACCCTCTCCGACCCGGCCGCCGCCGC
CATGCAGCCCTCCAGCCTTCTGCCGCTCGCCCTCTGCCTGCTGGCTGCAC
CCGCCTCCGCGCTCGTCAGGATCCCGCTGCACAAGTTCACGTCCATCCGC
CGGACCATGTCGGAGGTTGGGGGCTCTGTGGAGGACCTGATTGCCAAAGG
CCCCGTCTCAAAGTACTCCCAGGCGGTGCCAGCCGTGACCGAGGGGCCCA
TTCCCGAGGTGCTCAAGAACTACATGGACGCCCAGTACTACGGGGAGATT
GGCATCGGGACGCCCCCCCAGTGCTTCACAGTCGTCTTCGACACGGGCTC
CTCCAACCTGTGGGTCCCCTCCATCCACTGCAAACTGCTGGACATCGCTT
GCTGGATCCACCACAAGTACAACAGCGACAAGTCCAGCACCTACGTGAAG
AATGGTACCTCGTTTGACATCCACTATGGCTCGGGCAGCCTCTCCGGGTA
CCTGAGCCAGGACACTGTGTCGGTGCCCTGCCAGTCAGCGTCGTCAGCCT
CTGCCCTGGGCGGTGTCAAAGTGGAGAGGCAGGTCTTTGGGGAGGCCACC
AAGCAGCCAGGCATCACCTTCATCGCAGCCAAGTTCGATGGCATCCTGGG
CATGGCCTACCCCCGCATCTCCGTCAACAACGTGCTGCCCGTCTTCGACA
ACCTGATGCAGCAGAAGCTGGTGGACCAGAACATCTTCTCCTTCTACCTG
AGCAGGGACCCAGATGCGCAGCCTGGGGGTGAGCTGATGCTGGGTGGCAC
AGACTCCAAGTATTACAAGGGTTCTCTGTCCTACCTGAATGTCACCCGCA
AGGCCTACTGGCAGGTCCACCTGGACCAGGTGGAGGTGGCCAGCGGGCTG
ACCCTGTGCAAGGAGGGCTGTGAGGCCATTGTGGACACAGGCACTTCCCT
CATGGTGGGCCCGGTGGATGAGGTGCGCGAGCTGCAGAAGGCCATCGGGG
CCGTGCCGCTGATTCAGGGCGAGTACATGATCCCCTGTGAGAAGGTGTCC
ACCCTGCCCGCGATCACACTGAAGCTGGGAGGCAAAGGCTACAAGCTGTC
CCCAGAGGACTACACGCTCAAGGTGTCGCAGGCCGGGAAGACCCTCTGCC
TGAGCGGCTTCATGGGCATGGACATCCCGCCACCCAGCGGGCCACTCTGG
ATCCTGGGCGACGTCTTCATCGGCCGCTACTACACTGTGTTTGACCGTGA
CAACAACAGGGTGGGCTTCGCCGAGGCTGCCCGCCTCTAGTTCCCAAGGC
GTCCGCGCGCCAGCACAGAAACAGAGGAGAGTCCCAGAGCAGGAGGCCCC
TGGCCCAGCGGCCCCTCCCACACACACCCACACACTCGCCCGCCCACTGT
CCTGGGCGCCCTGGAAGCCGGCGGCCCAAGCCCGACTTGCTGTTTTGTTC
TGTGGTTTTCCCCTCCCTGGGTTCAGAAATGCTGCCTGCCTGTCTGTCTC
TCCATCTGTTTGGTGGGGGTAGAGCTGATCCAGAGCACAGATCTGTTTCG
TGCATTGGAAGACCCCACCCAAGCTTGGCAGCCGAGCTCGTGTATCCTGG
GGCTCCCTTCATCTCCAGGGAGTCCCCTCCCCGGCCCTACCAGCGCCCGC
TGGGCTGAGCCCCTACCCCACACCAGGCCGTCCTCCCGGGCCCTCCCTTG
GAAACCTGCCCTGCCTGAGGGCCCCTCTGCCCAGCTTGGGCCCAGCTGGG
CTCTGCCACCCTACCTGTTCAGTGTCCCGGGCCCGTTGAGGATGAGGCCG
CTAGAGGCCTGAGGATGAGCTGGAAGGAGTGAGAGGGGACAAAACCCACC
TTGTTGGAGCCTGCAGGGTGGTGCTGGGACTGAGCCAGTCCCAGGGGCAT
GTATTGGCCTGGAGGTGGGGTTGGGATTGGGGGCTGGTGCCAGCCTTCCT
CTGCAGCTGACCTCTGTTGTCCTCCCCTTGGGCGGCTGAGAGCCCCAGCT
GACATGGAAATACAGTTGTTGGCCTCCGGCCTCCCCTC

As used herein, the term “CTSL” refers to the gene encoding Cathepsin L1. The terms “CTSL” and “Cathepsin L1” include wild-type forms of the CTSL gene, as well as variants (e.g., splice variants and polymorphisms) of wild-type CTSL. Examples of such variants are nucleic acids having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to a wild-type CTSL nucleic acid sequence (e.g., SEQ ID NO: 23, ENA accession number X12451). SEQ ID NO: 23 is a wild-type gene sequence encoding CTSL protein, and is shown below:

(SEQ ID NO: 23)
AGAACCGCGACCTCCGCAACCTTGAGCGGCATCCGTGGAGTGCGCCTGCA
GCTACGACCGCAGCAGGAAAGCGCCGCCGGCCAGGCCCAGCTGTGGCCGG
ACAGGGACTGGAAGAGAGGACGCGGTCGAGTAGGTGTGCACCAGCCCTGG
CAACGAGAGCGTCTACCCCGAACTCTGCTGGCCTTGAGGTGGGGAAGCCG
GGGAGGGCAGTTGAGGACCCCGCGGAGGCGCGTGACTGGTTGAGCGGGCA
GGCCAGCCTCCGAGCCGGGTGGACACAGGTTTTAAAACATGAATCCTACA
CTCATCCTTGCTGCCTTTTGCCTGGGAATTGCCTCAGCTACTCTAACATT
TGATCACAGTTTAGAGGCACAGTGGACCAAGTGGAAGGCGATGCACAACA
GATTATACGGCATGAATGAAGAAGGATGGAGGAGAGCAGTGTGGGAGAAG
AACATGAAGATGATTGAACTGCACAATCAGGAATACAGGGAAGGGAAACA
CAGCTTCACAATGGCCATGAACGCCTTTGGAGACATGACCAGTGAAGAAT
TCAGGCAGGTGATGAATGGCTTTCAAAACCGTAAGCCCAGGAAGGGGAAA
GTGTTCCAGGAACCTCTGTTTTATGAGGCCCCCAGATCTGTGGATTGGAG
AGAGAAAGGCTACGTGACTCCTGTGAAGAATCAGGGTCAGTGTGGTTCTT
GTTGGGCTTTTAGTGCTACTGGTGCTCTTGAAGGACAGATGTTCCGGAAA
ACTGGGAGGCTTATCTCACTGAGTGAGCAGAATCTGGTAGACTGCTCTGG
GCCTCAAGGCAATGAAGGCTGCAATGGTGGCCTAATGGATTATGCTTTCC
AGTATGTTCAGGATAATGGAGGCCTGGACTCTGAGGAATCCTATCCATAT
GAGGCAACAGAAGAATCCTGTAAGTACAATCCCAAGTATTCTGTTGCTAA
TGACACCGGCTTTGTGGACATCCCTAAGCAGGAGAAGGCCCTGATGAAGG
CAGTTGCAACTGTGGGGCCCATTTCTGTTGCTATTGATGCAGGTCATGAG
TCCTTCCTGTTCTATAAAGAAGGCATTTATTTTGAGCCAGACTGTAGCAG
TGAAGACATGGATCATGGTGTGCTGGTGGTTGGCTACGGATTTGAAAGCA
CAGAATCAGATAACAATAAATATTGGCTGGTGAAGAACAGCTGGGGTGAA
GAATGGGGCATGGGTGGCTACGTAAAGATGGCCAAAGACCGGAGAAACCA
TTGTGGAATTGCCTCAGCAGCCAGCTACCCCACTGTGTGAGCTGGTGGAC
GGTGATGAGGAAGGACTTGACTGGGGATGGCGCATGCATGGGAGGAATTC
ATCTTCAGTCTACCAGCCCCCGCTGTGTCGGATACACACTCGAATCATTG
AAGATCCGAGTGTGATTTGAATTCTGTGATATTTTCACACTGGTAAATGT
TACCTCTATTTTAATTACTGCTATAAATAGGTTTATATTATTGATTCACT
TACTGACTTTGCATTTTCGTTTTTAAAAGGATGTATAAATTTTTACCTGT
TTAAATAAAATTTAATTTCAAATGT

As used herein, the term “CXCL10” refers to the gene encoding C—X-C motif chemokine 10. The terms “CXCL10” and “C—X-C motif chemokine 10” include wild-type forms of the CXCL10 gene, as well as variants (e.g., splice variants and polymorphisms) of wild-type CXCL10. Examples of such variants are nucleic acids having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to a wild-type CXCL10 nucleic acid sequence (e.g., SEQ ID NO: 24, ENA accession number X02530). SEQ ID NO: 24 is a wild-type gene sequence encoding CXCL10 protein, and is shown below:

(SEQ ID NO: 24)
GAGACATTCCTCAATTGCTTAGACATATTCTGAGCCTACAGCAGAGGAAC
CTCCAGTCTCAGCACCATGAATCAAACTGCGATTCTGATTTGCTGCCTTA
TCTTTCTGACTCTAAGTGGCATTCAAGGAGTACCTCTCTCTAGAACCGTA
CGCTGTACCTGCATCAGCATTAGTAATCAACCTGTTAATCCAAGGTCTTT
AGAAAAACTTGAAATTATTCCTGCAAGCCAATTTTGTCCACGTGTTGAGA
TCATTGCTACAATGAAAAAGAAGGGTGAGAAGAGATGTCTGAATCCAGAA
TCGAAGGCCATCAAGAATTTACTGAAAGCAGTTAGCAAGGAAATGTCTAA
AAGATCTCCTTAAAACCAGAGGGGAGCAAAATCGATGCAGTGCTTCCAAG
GATGGACCACACAGAGGCTGCCTCTCCCATCACTTCCCTACATGGAGTAT
ATGTCAAGCCATAATTGTTCTTAGTTTGCAGTTACACTAAAAGGTGACCA
ATGATGGTCACCAAATCAGCTGCTACTACTCCTGTAGGAAGGTTAATGTT
CATCATCCTAAGCTATTCAGTAATAACTCTACCCTGGCACTATAATGTAA
GCTCTACTGAGGTGCTATGTTCTTAGTGGATGTTCTGACCCTGCTTCAAA
TATTTCCCTCACCTTTCCCATCTTCCAAGGGTACTAAGGAATCTTTCTGC
TTTGGGGTTTATCAGAATTCTCAGAATCTCAAATAACTAAAAGGTATGCA
ATCAAATCTGCTTTTTAAAGAATGCTCTTTACTTCATGGACTTCCACTGC
CATCCTCCCAAGGGGCCCAAATTCTTTCAGTGGCTACCTACATACAATTC
CAAACACATACAGGAAGGTAGAAATATCTGAAAATGTATGTGTAAGTATT
CTTATTTAATGAAAGACTGTACAAAGTATAAGTCTTAGATGTATATATTT
CCTATATTGTTTTCAGTGTACATGGAATAACATGTAATTAAGTACTATGT
ATCAATGAGTAACAGGAAAATTTTAAAAATACAGATAGATATATGCTCTG
CATGTTACATAAGATAAATGTGCTGAATGGTTTTCAAATAAAAATGAGGT
ACTCTCCTGGAAATATTAAGAAAGACTATCTAAATGTTGAAAGATCAAAA
GGTTAATAAAGTAATTATAACT

As used herein, the term “CXCL13” refers to the gene encoding C—X-C motif chemokine 13. The terms “CXCL13” and “C—X-C motif chemokine 13” include wild-type forms of the CXCL13 gene, as well as variants (e.g., splice variants and polymorphisms) of wild-type CXCL13. Examples of such variants are nucleic acids having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to a wild-type CXCL13 nucleic acid sequence (e.g., SEQ ID NO: 25, ENA accession number AF044197). SEQ ID NO: 25 is a wild-type gene sequence encoding CXCL13 protein, and is shown below:

(SEQ ID NO: 25)
TTCGGCACTTGGGAGAAGATGTTTGAAAAAACTGACTCTGCTAATGAGCC
TGGACTCAGAGCTCAAGTCTGAACTCTACCTCCAGACAGAATGAAGTTCA
TCTCGACATCTCTGCTTCTCATGCTGCTGGTCAGCAGCCTCTCTCCAGTC
CAAGGTGTTCTGGAGGTCTATTACACAAGCTTGAGGTGTAGATGTGTCCA
AGAGAGCTCAGTCTTTATCCCTAGACGCTTCATTGATCGAATTCAAATCT
TGCCCCGTGGGAATGGTTGTCCAAGAAAAGAAATCATAGTCTGGAAGAAG
AACAAGTCAATTGTGTGTGTGGACCCTCAAGCTGAATGGATACAAAGAAT
GATGGAAGTATTGAGAAAAAGAAGTTCTTCAACTCTACCAGTTCCAGTGT
TTAAGAGAAAGATTCCCTGATGCTGATATTTCCACTAAGAACACCTGCAT
TCTTCCCTTATCCCTGCTCTGGATTTTAGTTTTGTGCTTAGTTAAATCTT
TTCCAGGGAGAAAGAACTTCCCCATACAAATAAGGCATGAGGACTATGTG
AAAAATAACCTTGCAGGAGCTGATGGGGCAAACTCAAGCTTCTTCACTCA
CAGCACCCTATATACACTTGGAGTTTGCATTCTTATTCATCAGGGAGGAA
AGTTTCTTTGAAAATAGTTATTCAGTTATAAGTAATACAGGATTATTTTG
ATTATATACTTGTTGTTTAATGTTTAAAATTTCTTAGAAAACAATGGAAT
GAGAATTTAAGCCTCAAATTTGAACATGTGGCTTGAATTAAGAAGAAAAT
TATGGCATATATTAAAAGCAGGCTTCTATGAAAGACTCAAAAAGCTGCCT
GGGAGGCAGATGGAACTTGAGCCTGTCAAGAGGCAAAGGAATCCATGTAG
TAGATATCCTCTGCTTAAAAACTCACTACGGAGGAGAATTAAGTCCTACT
TTTAAAGAATTTCTTTATAAAATTTACTGTCTAAGATTAATAGCATTCGA
AGATCCCCAGACTTCATAGAATACTCAGGGAAAGCATTTAAAGGGTGATG
TACACATGTATCCTTTCACACATTTGCCTTGACAAACTTCTTTCACTCAC
ATCTTTTTCACTGACTTTTTTTGTGGGGGCGGGGCCGGGGGGACTCTGGT
ATCTAATTCTTTAATGATTCCTATAAATCTAATGACATTCAATAAAGTTG
AGCAAACATTTTACTT

As used herein, the term “DSG2” refers to the gene encoding Desmoglein 2. The terms “DSG2” and “Desmoglein 2” include wild-type forms of the DSG2 gene, as well as variants (e.g., splice variants and polymorphisms) of wild-type DSG2. Examples of such variants are nucleic acids having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to a wild-type DSG2 nucleic acid sequence (e.g., SEQ ID NO: 26, NCBI Reference Sequence: NM_001943.4). SEQ ID NO: 26 is a wild-type gene sequence encoding DSG2 protein, and is shown below:

(SEQ ID NO: 26)
CCACCTCTGTAAAAGCGGCCCGGGCCGGCCCCCGGCTCCATTTTCTCGCGGCGGCCACACCTGGA
GCCGCGCCTTTGGGTTGGGCTGGGCTGGGCCGCGCAACCGCCACGGGAAGACAGCCCTCGGGGC
GGGGAGGGAGAGGGTGGCCGGGCCGGGGGGAGGCCGGGGCCAGGGAGGAGCCGAGTGCGCGC
TCGGGGCAGGCGGCGGCGCGGAGCGGTGCGGCGGCGGGAGGCGGAGGCGAGGGTGCGATGGC
GCGGAGCCCGGGACGCGCGTACGCCCTGCTGCTTCTCCTGATCTGCTTTAACGTTGGAAGTGGACT
TCACTTACAGGTCTTAAGCACAAGAAATGAAAATAAGCTGCTTCCTAAACATCCTCATTTAGTGCGGC
AAAAGCGCGCCTGGATCACCGCCCCCGTGGCTCTTCGGGAGGGAGAGGATCTGTCCAAGAAGAAT
CCAATTGCCAAGATACATTCTGATCTTGCAGAAGAAAGAGGACTCAAAATTACTTACAAATACACTGG
AAAAGGGATTACAGAGCCACCTTTTGGTATATTTGTCTTTAACAAAGATACTGGAGAACTGAATGTTA
CCAGCATTCTTGATCGAGAAGAAACACCATTTTTTCTGCTAACAGGTTACGCTTTGGATGCAAGAGGA
AACAATGTAGAGAAACCCTTAGAGCTACGCATTAAGGTTCTTGATATCAATGACAACGAACCAGTGTT
CACACAGGATGTCTTTGTTGGGTCTGTTGAAGAGTTGAGTGCAGCACATACTCTTGTGATGAAAATCA
ATGCAACAGATGCAGATGAGCCCAATACCCTGAATTCGAAAATTTCCTATAGAATCGTATCTCTGGAG
CCTGCTTATCCTCCAGTGTTCTACCTAAATAAAGATACAGGAGAGATTTATACAACCAGTGTTACCTT
GGACAGAGAGGAACACAGCAGCTACACTTTGACAGTAGAAGCAAGAGATGGCAATGGAGAAGTTAC
AGACAAACCTGTAAAACAAGCTCAAGTTCAGATTCGTATTTTGGATGTCAATGACAATATACCTGTAG
TAGAAAATAAAGTGCTTGAAGGGATGGTTGAAGAAAATCAAGTCAACGTAGAAGTTACGCGCATAAA
AGTGTTCGATGCAGATGAAATAGGTTCTGATAATTGGCTGGCAAATTTTACATTTGCATCAGGAAATG
AAGGAGGTTATTTCCACATAGAAACAGATGCTCAAACTAACGAAGGAATTGTGACCCTTATTAAGGAA
GTAGATTATGAAGAAATGAAGAATCTTGACTTCAGTGTTATTGTCGCTAATAAAGCAGCTTTTCACAA
GTCGATTAGGAGTAAATACAAGCCTACACCCATTCCCATCAAGGTCAAAGTGAAAAATGTGAAAGAA
GGCATTCATTTTAAAAGCAGCGTCATCTCAATTTATGTTAGCGAGAGCATGGATAGATCAAGCAAAGG
CCAAATAATTGGAAATTTTCAAGCTTTTGATGAGGACACTGGACTACCAGCCCATGCAAGATATGTAA
AATTAGAAGATAGAGATAATTGGATCTCTGTGGATTCTGTCACATCTGAAATTAAACTTGCAAAACTTC
CTGATTTTGAATCTAGATATGTTCAAAATGGCACATACACTGTAAAGATTGTGGCCATATCAGAAGATT
ATCCTAGAAAAACCATCACTGGCACAGTCCTTATCAATGTTGAAGACATCAACGACAACTGTCCCACA
CTGATAGAGCCTGTGCAGACAATCTGTCACGATGCAGAGTATGTGAATGTTACTGCAGAGGACCTGG
ATGGACACCCAAACAGTGGCCCTTTCAGTTTCTCCGTCATTGACAAACCACCTGGCATGGCAGAAAA
ATGGAAAATAGCACGCCAAGAAAGTACCAGTGTGCTGCTGCAACAAAGTGAGAAAAAGCTTGGGAG
AAGTGAAATTCAGTTCCTGATTTCAGACAATCAGGGTTTTAGTTGTCCTGAAAAGCAGGTCCTTACAC
TCACAGTTTGTGAGTGTCTGCATGGCAGCGGCTGCAGGGAAGCACAGCATGACTCCTATGTGGGCC
TGGGACCCGCAGCAATTGCGCTCATGATTTTGGCCTTTCTGCTCCTGCTATTGGTACCACTTTTACTG
CTGATGTGCCATTGCGGAAAGGGCGCCAAAGGCTTTACCCCCATACCTGGCACCATAGAGATGCTG
CATCCTTGGAATAATGAAGGAGCACCACCTGAAGACAAGGTGGTGCCATCATTTCTGCCAGTGGATC
AAGGGGGCAGTCTAGTAGGAAGAAATGGAGTAGGAGGTATGGCCAAGGAAGCCACGATGAAAGGA
AGTAGCTCTGCTTCCATTGTCAAAGGGCAACATGAGATGTCCGAGATGGATGGAAGGTGGGAAGAA
CACAGAAGCCTGCTTTCTGGTAGAGCTACCCAGTTTACAGGGGCCACAGGCGCTATCATGACCACT
GAAACCACGAAGACCGCAAGGGCCACAGGGGCTTCCAGAGACATGGCCGGAGCTCAGGCAGCTGC
TGTTGCACTGAACGAAGAATTCTTAAGAAATTATTTCACTGATAAAGCGGCCTCTTACACTGAGGAAG
ATGAAAATCACACAGCCAAAGATTGCCTTCTGGTTTATTCTCAGGAAGAAACTGAATCGCTGAATGCT
TCTATTGGTTGTTGCAGTTTTATTGAAGGAGAGCTAGATGACCGCTTCTTAGATGATTTGGGACTTAA
ATTCAAGACACTAGCTGAAGTTTGCCTGGGTCAAAAAATAGATATAAATAAGGAAATTGAGCAGAGAC
AAAAACCTGCCACAGAAACAAGTATGAACACAGCTTCACATTCACTCTGTGAGCAAACTATGGTTAAT
TCAGAGAATACCTACTCCTCTGGCAGTAGCTTCCCAGTTCCAAAATCTTTGCAAGAAGCCAATGCAG
AGAAAGTAACTCAGGAAATAGTCACTGAAAGATCTGTGTCTTCTAGGCAGGCGCAAAAGGTAGCTAC
ACCTCTTCCTGACCCAATGGCTTCTAGAAATGTGATAGCAACAGAAACTTCCTATGTCACAGGGTCCA
CTATGCCACCAACCACTGTGATCCTGGGTCCTAGCCAGCCACAGAGCCTTATTGTGACAGAGAGGG
TGTATGCTCCAGCTTCTACCTTGGTAGATCAGCCTTATGCTAATGAAGGTACAGTTGTGGTCACTGAA
AGAGTAATACAGCCTCATGGGGGTGGATCGAATCCTCTGGAAGGCACTCAGCATCTTCAAGATGTAC
CTTACGTCATGGTGAGGGAAAGAGAGAGCTTCCTTGCCCCCAGCTCAGGTGTGCAGCCTACTCTGG
CCATGCCTAATATAGCAGTAGGACAGAATGTGACAGTGACAGAAAGAGTTCTAGCACCTGCTTCCAC
TCTGCAATCCAGTTACCAGATTCCCACTGAAAATTCTATGACGGCTAGGAACACCACGGTGTCTGGA
GCTGGAGTCCCTGGCCCTCTGCCAGATTTTGGTTTAGAGGAATCTGGTCATTCTAATTCTACCATAAC
CACATCTTCCACCAGAGTTACCAAGCATAGCACTGTACAGCATTCTTACTCCTAAACAGCAGTCAGCC
ACAAACTGACCCAGAGTTTAATTAGCAGTGACTAATTTCATGTTTCCAATGTACCTGATTTTTCATGAG
CCTTACAGACACACAGAGACACATACACATTGATCTTAAAATTTTTCTCAGTCACTGATATGCAAAGG
ACCACACTGTCTCTGCTTCCAGGAGTATTTTAGAAATGTTCCACAATTTACTGAAGACATAGAGATGA
TGCTGCTGCTTAGGTGCCTTTTAGCAAGCTATGCAAACAATCCTGATAAAACAAGATACATAGAGAGT
CAATCTGGCTTCTGAGAATTTACCAAGTGAACAGAGTACCTAGTTCATCAGCCGTCCAGTAAAGCAA
CCCAGGAAACTGACTGGGTCTCTTTGCCTACCGTATTAACATTAAACATTGATGTTCTGTATTCTGTA
CTTTACTGCACCCAGCAGACTTTCAACAACTCATTGATCCAAAGATACATGCACAGTCTGAGCACCAG
CTATGGTGCTCATAACTTCTTTAAGACTTGAACCCTTTCAATCTGTGTGATTCATTAAATTGGACCATT
GATGATAAGAATACACATTGTATGTTTCTGTGCACATGACAGTGTGTGTGTGTGCACGTACATACTGT
ATAGTCTTAAAAATAGCATTATACTGGCCAGGGGTGGTGGCTAACGCCTGTAATCCCAGCACTTTGG
GAGGCCGAGGCGGGTGGATCAACTGTGGTCAGGAGTTTGAGATCAGCCAGGCCAACCTGGTGAAA
CCCCGTCTCTACTAAAAATACAAAAATTAGCTGGGCGTGATGGTGGGCGCCTGTAATCCCAGCTACT
TGGGAGGCTGAGGCAGGAGAATCACTTGAACCCGGGAGGCGGAGGTTGCAGTGAGCCGAGATCGC
ACCATTGCACTCCAGTCTGGGCAACAGAGTGAGATTCCGTCTCAAAAAAAAAAAGAAAAGGAAAAAA
AAATAGCATTATACCTCTTCCTTGTCTCAACCGCCATGAAAATTCTGAACACTCCAAATTCAGTTGAAT
AATCCAAAACAAAATTTATAAGTATAAAATAATTTTACTTCTTATAGTAATAGTATACTTTAAAAAGCCT
CAGGGTATATTATOTTCTAAACAGCTACAATTCAGTGCAGCTACATTAACCAACTATGTTCTCTAGTTG
AGAACAACTAGGCCTATTTCACTGCTGTGTAGCCTCAGTGCCTAACATGGGTGCCAAATAAATATTCG
TAGAATTACACTGAATTGTAAAAACCATTCGTTTTTGTTTACAATTGCCAAAAATCTCAAAAGGCCCTG
TATTTATGTAATTCTTTGAAATTATTATTTTATTTTGATTTCTCAGTTATTGACTGGCTGGGTGTGACTT
AGTACATAAGTACTCAATATTATAAAAACCTCAAATAATTGACTTGATTTTACACAACATCCTTCCCTTT
TCTACAAGTTAATTTTTTTACAAATCATTTGGGTTATCTCCTAAATAGGTTATATTTTATTGCTTCTAGA
AACAATGTTTCAAAATATATGTGCATTATCAGTAATAATTTGTATAAATATTTCCCACAACAATTTTCAT
AATTTTCAAAGACTAATTTCTTGACTGAAGATATTTTGCTAGGGAAGTGAAACTTTAAAATTTTGTAGA
TTTTAAAAAATATTGTTGAATGGTGTCATGCAAAGGATTTATATAGTGTGCTCCCACTAACTGTACAGA
TCAGGACACATATTTTTAGACATCTAAGTCTGTAGCTTAAATGGAGGTTACTCTTCCATCATCTAGAAT
TGTTTACTTAGTAATTGTTGTTTCTTTTATTATTATAGACTTACTATCAGTTTTATTTTGCCAAGTATGCA
ACAGGTATATCACTAGTATATGAAAATGTAAATATCACTTGTGTACTCAAACAAAAGTTGGTCTTAAGC
TTCCACCTTGAGCAGCCTTGGAAACCTAACCTGCCTCTTTTAGCATAATCACATTTTCTAAATGATTTT
CTTTGTTCCTGAAAAAGTGATTTGTATTAGTTTTACATTTGTTTTTTGGAAGATTATATTTGTATATGTA
TCATCATAAAATATTTAAATAAAAAGTATCTTTAGAGTGACCCTTTCCCCATAGATTTTTATTTCTCTAT
TATATTTTACAAGGAATATAACTCAGTTTGTTAGGGAGAGTGCCTTAAAGGCAGGTGTTTCTTGGACT
TTGTTATTTAATTAGATCTGCTTGCAATAAAAAAAGTTGTCGGTTATCTAAAATTCAAAAAAAAAAAAAA
AAAA

As used herein, the term “ECHDC3” refers to the gene encoding Enoyl-CoA Hydratase Domain Containing 3. The terms “ECHDC” and “Enoyl-CoA Hydratase Domain Containing 3” include wild-type forms of the ECHDC gene, as well as variants (e.g., splice variants and polymorphisms) of wild-type ECHDC. Examples of such variants are nucleic acids having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to a wild-type ECHDC nucleic acid sequence (e.g., SEQ ID NO: 27, NCBI Reference Sequence: NM_024693.4). SEQ ID NO: 27 is a wild-type gene sequence encoding ECHDC protein, and is shown below:

(SEQ ID NO: 27)
GGGGCGGGGCGTGCCGGGGGGGGCGTAGTACGGACTGGGCCTGGCCTGGG
GCGTCCCCGCGAAGCCTGGGCCTGTCAGGCGGTTCCGTCCGGGTCTCGGC
CACCGTCGAGTTCCGTCGAGTTCCGTCCCGGCCCTGCTCACAGCAGCGCC
CTCGGAGCGCCCAGCACCTGCGGCCGGCCAGGCAGCGCGATCCTGCGGCG
TCTGGCCATCCCGAATGCTATGGCCGCCGTCGCCGTCTTGCGGGCCTTCG
GGGCAAGTGGGCCCATGTGTCTCCGGCGCGGCCCCTGGGCCCAGCTCCCC
GCCCGCTTCTGCAGCCGGGACCCGGCCGGGGGGGGGGGGCGGGAGTCGGA
GCCGCGGCCCACCAGCGCGCGGCAGCTGGACGGCATAAGGAACATCGTCT
TGAGCAATCCCAAGAAGAGGAACACGTTGTCACTTGCAATGCTGAAATCT
CTCCAAAGTGACATTCTTCATGACGCTGACAGCAACGATCTGAAAGTCAT
TATCATCTCGGCTGAGGGGCCTGTGTTTTCTTCTGGGCATGACTTAAAGG
AGCTGACAGAGGAGCAAGGCCGTGATTACCATGCCGAAGTATTTCAGACC
TGTTCCAAGGTCATGATGCACATCCGGAACCACCCCGTCCCCGTCATTGC
CATGGTCAATGGCCTGGCCACGGCTGCCGGCTGTCAACTGGTTGCCAGCT
GCGACATTGCCGTGGCGAGCGACAAGTCCTCTTTTGCCACTCCTGGGGTG
AACGTCGGGCTCTTCTGTTCTACCCCTGGGGTTGCCTTGGCAAGAGCAGT
GCCTAGAAAGGTGGCCTTGGAGATGCTCTTTACTGGTGAGCCCATTTCTG
CCCAGGAGGCCCTGCTCCACGGGCTGCTTAGCAAGGTGGTGCCAGAGGCG
GAGCTGCAGGAGGAGACCATGCGGATCGCTAGGAAGATCGCATCGCTGAG
CCGTCCGGTGGTGTCCCTGGGCAAAGCCACCTTCTACAAGCAGCTGCCCC
AGGACCTGGGGACGGCTTACTACCTCACCTCCCAGGCCATGGTGGACAAC
CTGGCCCTGCGGGACGGGCAGGAGGGCATCACGGCCTTCCTCCAGAAGAG
AAAACCTGTCTGGTCACACGAGCCAGTGTGAGTGGAGGCAGAGGAGTGAG
GCCCACGGGCAGCGCCCAGGAGCCCACCTTCCCCTCTGGCCCAGCCACCA
CTGCCTCTCAGCTTCAACAGGTGACAGGCTGCTTTCGTGACTTGATATTG
GTGTCATAGCATTTGGCCTACATTAAAAGCCACAATTTCATGGGGAAAGG
ACAAAATGGAGAGTGACTGAGGTGCTGACCTCAGTGCAAGGCTGGTGAAC
CCTGCAGCGGGCCAGCTATGGTGGGAAGCCTGGCATTTGGGGTGCTCCTT
GCAACGTCTTAAGCAAGCGACCCCCCTGACATAGCAAAAGGTGGCAACCC
ATGGAGGCAGAAAGAAGGACGCCAGCCTGACCCTTATCTGAAACGTCCTA
AGCAGAGTTAATCCTGGCTGCTCAGGAGAGGCGACACATTTCAAATCTCC
ACGAGATATTCTCCACACAGAAAATCTTCTTGATTCTATAGAGACTTAAT
CATGCCTATGGCTTTGAATAATCTTATGTGATTTAAATAAATTAAATCTT
TATAAAAAAAAAAAAAAAAAAAA

As used herein, the term “EPHA1” refers to the gene encoding Ephrin type-A receptor 1. The terms “EPHA1” and “Ephrin type-A receptor 1” include wild-type forms of the EPHA1 gene, as well as variants (e.g., splice variants and polymorphisms) of wild-type EPHA1. Examples of such variants are nucleic acids having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to a wild-type EPHA1 nucleic acid sequence (e.g., SEQ ID NO: 28, ENA accession number M18391). SEQ ID NO: 28 is a wild-type gene sequence encoding EPHA1 protein, and is shown below:

(SEQ ID NO: 57)
GCCCCCGCCCGGCCCGCCCCGCTCTCCTAGTCCCTTGCAACCTGGCGCTG
CATCCGGGCCACTGTCCCAGGTCCCAGGTCCCGGCCCGGAGCTATGGAGC
GGCGCTGGCCCCTGGGGCTAGGGCTGGTGCTGCTGCTCTGCGCCCCGCTG
CCCCCGGGGGCGCGCGCCAAGGAAGTTACTCTGATGGACACAAGCAAGGC
ACAGGGAGAGCTGGGCTGGCTGCTGGATCCCCCAAAAGATGGGTGGAGTG
AACAGCAACAGATACTGAATGGGACACCCCTCTACATGTACCAGGACTGC
CCAATGCAAGGACGCAGAGACACTGACCACTGGCTTCGCTCCAATTGGAT
CTACCGCGGGGAGGAGGCTTCCCGCGTCCACGTGGAGCTGCAGTTCACCG
TGCGGGACTGCAAGAGTTTCCCTGGGGGAGCCGGGCCTCTGGGCTGCAAG
GAGACCTTCAACCTTCTGTACATGGAGAGTGACCAGGATGTGGGCATTCA
GCTCCGACGGCCCTTGTTCCAGAAGGTAACCACGGTGGCTGCAGACCAGA
GCTTCACCATTCGAGACCTTGCGTCTGGCTCCGTGAAGCTGAATGTGGAG
CGCTGCTCTCTGGGCCGCCTGACCCGCCGTGGCCTCTACCTCGCTTTCCA
CAACCCGGGTGCCTGTGTGGCCCTGGTGTCTGTCCGGGTCTTCTACCAGC
GCTGTCCTGAGACCCTGAATGGCTTGGCCCAATTCCCAGACACTCTGCCT
GGCCCCGCTGGGTTGGTGGAAGTGGCGGGCACCTGCTTGCCCCACGCGCG
GGCCAGCCCCAGGCCCTCAGGTGCACCCCGCATGCACTGCAGCCCTGATG
GCGAGTGGCTGGTGCCTGTAGGACGGTGCCACTGTGAGCCTGGCTATGAG
GAAGGTGGCAGTGGCGAAGCATGTGTTGCCTGCCCTAGCGGCTCCTACCG
GATGGACATGGACACACCCCATTGTCTCACGTGCCCCCAGCAGAGCACTG
CTGAGTCTGAGGGGGCCACCATCTGTACCTGTGAGAGCGGCCATTACAGA
GCTCCCGGGGAGGGCCCCCAGGTGGCATGCACAGGTCCCCCCTCGGCCCC
CCGAAACCTGAGCTTCTCTGCCTCAGGGACTCAGCTCTCCCTGCGTTGGG
AACCCCCAGCAGATACGGGGGGACGCCAGGATGTCAGATACAGTGTGAGG
TGTTCCCAGTGTCAGGGCACAGCACAGGACGGGGGGCCCTGCCAGCCCTG
TGGGGTGGGCGTGCACTTCTCGCCGGGGGCCCGGGCGCTCACCACACCTG
CAGTGCATGTCAATGGCCTTGAACCTTATGCCAACTACACCTTTAATGTG
GAAGCCCAAAATGGAGTGTCAGGGCTGGGCAGCTCTGGCCATGCCAGCAC
CTCAGTCAGCATCAGCATGGGGCATGCAGAGTCACTGTCAGGCCTGTCTC
TGAGACTGGTGAAGAAAGAACCGAGGCAACTAGAGCTGACCTGGGCGGGG
TCCCGGCCCCGAAGCCCTGGGGCGAACCTGACCTATGAGCTGCACGTGCT
GAACCAGGATGAAGAACGGTACCAGATGGTTCTAGAACCCAGGGTCTTGC
TGACAGAGCTGCAGCCTGACACCACATACATCGTCAGAGTCCGAATGCTG
ACCCCACTGGGTCCTGGCCCTTTCTCCCCTGATCATGAGTTTCGGACCAG
CCCACCAGTGTCCAGGGGCCTGACTGGAGGAGAGATTGTAGCCGTCATCT
TTGGGCTGCTGCTTGGTGCAGCCTTGCTGCTTGGGATTCTCGTTTTCCGG
TCCAGGAGAGCCCAGCGGCAGAGGCAGCAGAGGCACGTGACCGCGCCACC
GATGTGGATCGAGAGGACAAGCTGTGCTGAAGCCTTATGTGGTACCTCCA
GGCATACGAGGACCCTGCACAGGGAGCCTTGGACTTTACCCGGAGGCTGG
TCTAATTTTCCTTCCCGGGAGCTTGATCCAGCGTGGCTGATGGTGGACAC
TGTCATAGGAGAAGGAGAGTTTGGGGAAGTGTATCGAGGGACCCTCAGGC
TCCCCAGCCAGGACTGCAAGACTGTGGCCATTAAGACCTTAAAAGACACA
TCCCCAGGTGGCCAGTGGTGGAACTTCCTTCGAGAGGCAACTATCATGGG
CCAGTTTAGCCACCCGCATATTCTGCATCTGGAAGGCGTCGTCACAAAGC
GAAAGCCGATCATGATCATCACAGAATTTATGGAGAATGCAGCCCTGGAT
GCCTTCCTGAGGGAGCGGGAGGACCAGCTGGTCCCTGGGCAGCTAGTGGC
CATGCTGCAGGGCATAGCATCTGGCATGAACTACCTCAGTAATCACAATT
ATGTCCACCGGGACCTGGCTGCCAGAAACATCTTGGTGAATCAAAACCTG
TGCTGCAAGGTGTCTGACTTTGGCCTGACTCGCCTCCTGGATGACTTTGA
TGGCACATACGAAACCCAGGGAGGAAAGATCCCTATCCGTTGGACAGCCC
CTGAAGCCATTGCCCATCGGATCTTCACCACAGCCAGCGATGTGTGGAGC
TTTGGGATTGTGATGTGGGAGGTGCTGAGCTTTGGGGACAAGCCTTATGG
GGAGATGAGCAATCAGGAGGTTATGAAGAGCATTGAGGATGGGTACCGGT
TGCCCCCTCCTGTGGACTGCCCTGCCCCTCTGTATGAGCTCATGAAGAAC
TGCTGGGCATATGACCGTGCCCGCCGGCCACACTTCCAGAAGCTTCAGGC
ACATCTGGAGCAACTGCTTGCCAACCCCCACTCCCTGCGGACCATTGCCA
ACTTTGACCCCAGGGTGACTCTTCGCCTGCCCAGCCTGAGTGGCTCAGAT
GGGATCCCGTATCGAACCGTCTCTGAGTGGCTCGAGTCCATACGCATGAA
ACGCTACATCCTGCACTTCCACTCGGCTGGGCTGGACACCATGGAGTGTG
TGCTGGAGCTGACCGCTGAGGACCTGACGCAGATGGGAATCACACTGCCC
GGGCACCAGAAGCGCATTCTTTGCAGTATTCAGGGATTCAAGGACTGATC
CCTCCTCTCACCCCATGCCCAATCAGGGTGCAAGGAGCAAGGACGGGGCC
AAGGTCGCTCATGGTCACTCCCTGCGCCCCTTCCCACAACCTGCCAGACT
AGGCTATCGGTGCTGCTTCTGCCCGCTTTAAGGAGAACCCTGCTCTGCAC
CCCAGAAAACCTCTTTGTTTTAAAAGGGAGGTGGGGGTAGAAGTAAAAGG
ATGATCATGGGAGGGAGCTCAGGGGTTAATATATATACATACATACACAT
ATATATATTGTTGTAAATAAACAGGAAATGATTTTCTGCCTCCATCCCAC
CCATCAGGGCTGCAGGCACT

As used herein, the term “FABP5” refers to the gene encoding Fatty acid-binding protein 5. The terms “FABP5” and “Fatty acid-binding protein 5” include wild-type forms of the FABP5 gene, as well as variants (e.g., splice variants and polymorphisms) of wild-type FABP5. Examples of such variants are nucleic acids having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to a wild-type FABP5 nucleic acid sequence (e.g., SEQ ID NO: 29, ENA accession number M94856). SEQ ID NO: 29 is a wild-type gene sequence encoding FABP5 protein, and is shown below:

(SEQ ID NO: 29)
ACCGCCGACGCAGACCCCTCTCTGCACGCCAGCCCGCCCGCACCCACCAT
GGCCACAGTTCAGCAGCTGGAAGGAAGATGGCGCCTGGTGGACAGCAAAG
GCTTTGATGAATACATGAAGGAGCTAGGAGTGGGAATAGCTTTGCGAAAA
ATGGGCGCAATGGCCAAGCCAGATTGTATCATCACTTGTGATGGTAAAAA
CCTCACCATAAAAACTGAGAGCACTTTGAAAACAACACAGTTTTCTTGTA
CCCTGGGAGAGAAGTTTGAAGAAACCACAGCTGATGGCAGAAAAACTCAG
ACTGTCTGCAACTTTACAGATGGTGCATTGGTTCAGCATCAGGAGTGGGA
TGGGAAGGAAAGCACAATAACAAGAAAATTGAAAGATGGGAAATTAGTGG
TGGAGTGTGTCATGAACAATGTCACCTGTACTCGGATCTATGAAAAAGTA
GAATAAAAATTCCATCATCACTTTGGACAGGAGTTAATTAAGAGAATGAC
CAAGCTCAGTTCAATGAGCAAATCTCCATACTGTTTCTTTCTTTTTTTTT
TCATTACTGTGTTCAATTATCTTTATCATAAACATTTTACATGCAGCTAT
TTCAAAGTGTGTTGGATTAATTAGGATCATCCCTTTGGTTAATAAATAAA
TGTGTTTGTGCT

As used herein, the term “FERMT2” refers to the gene encoding Fermitin family homolog 2. The terms “FERMT2” and “Fermitin family homolog 2” include wild-type forms of the FERMT2 gene, as well as variants (e.g., splice variants and polymorphisms) of wild-type FERMT2. Examples of such variants are nucleic acids having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to a wild-type FERMT2 nucleic acid sequence (e.g., SEQ ID NO: 30, ENA accession number Z24725). SEQ ID NO: 30 is a wild-type gene sequence encoding FERMT2 protein, and is shown below:

(SEQ ID NO: 30)
CAAAAAGTGTGTGGAAAGGTGGATTGAGGGAGCGGGACCCCCGCGGGACC
CGAGGGGGCGGCAGGCGGGGAACGGGGAGTCAGCCCGCGCTGTGTCTCGG
GGCCGGCCGGCAGGAAGGAGCCATGGCTCTGGACGGGATAAGGATGCCAG
ATGGCTGCTACGCGGACGGGACGTGGGAACTGAGTGTCCATGTGACGGAC
CTGAACCGCGATATCACCCTGAGAGTGACCGGCGAGGTGCACATTGGAGG
CGTGATGCTTAAGCTGGTGGAGAAACTCGATGTAAAAAAAGATTGGTCTG
ACCATGCTCTCTGGTGGGAAAAGAAGAGAACTTGGCTTCTGAAGACACAT
TGGACCTTAGATAAGTATGGTATTCAGGCAGATGCTAAGCTTCAGTTCAC
CCCTCAGCACAAACTGCTCCGCCTGCAGCTTCCCAACATGAAGTATGTGA
AGGTGAAAGTGAATTTCTCTGATAGAGTCTTCAAAGCTGTTTCTGACATC
TGTAAGACTTTTAATATCAGACACCCCGAAGAACTTTCTCTCTTAAAGAA
ACCCAGAGATCCAACAAAGAAAAAAAAGAAGAAGCTAGATGACCAGTCTG
AAGATGAGGCACTTGAATTAGAGGGGCCTCTTATCACTCCTGGATCAGGA
AGTATATATTCAAGCCCAGGACTGTATAGTAAAACAATGACCCCCACTTA
TGATGCTCATGATGGAAGCCCCTTGTCACCAACTTCTGCTTGGTTTGGTG
ACAGTGCTTTGTCAGAAGGCAATCCTGGTATACTTGCTGTCAGTCAACCA
ATCACGTCACCAGAAATCTTGGCAAAAATGTTCAAGCCTCAAGCTCTTCT
TGATAAAGCAAAAATCAACCAAGGATGGCTTGATTCCTCAAGATCTCTCA
TGGAACAAGATGTGAAGGAAAATGAGGCCTTGCTGCTCCGATTCAAGTAT
TACAGCTTTTTTGATTTGAATCCAAAGTATGATGCAATCAGAATCAATCA
GCTTTATGAGCAGGCCAAATGGGCCATTCTCCTGGAAGAGATTGAATGCA
CAGAAGAAGAAATGATGATGTTTGCAGCCCTGCAGTATCATATCAATAAG
CTGTCAATCATGACATCAGAGAATCATTTGAACAACAGTGACAAAGAAGT
TGATGAAGTTGATGCTGCCCTTTCAGACCTGGAGATTACTCTGGAAGGGG
GTAAAACGTCAACAATTTTGGGTGACATTACTTCCATTCCTGAACTTGCT
GACTACATTAAAGTTTTCAAGCCAAAAAAGCTGACTCTGAAAGGTTACAA
ACAATATTGGTGCACCTTCAAAGACACATCCATTTCTTGTTATAAGAGCA
AAGAAGAATCCAGTGGCACACCAGCTCATCAGATGAACCTCAGGGGATGT
GAAGTTACCCCAGATGTAAACATTTCAGGCCAAAAATTTAACATTAAACT
CCTGATTCCAGTTGCAGAAGGCATGAATGAAATCTGGCTTCGTTGTGACA
ATGAAAAACAGTATGCACACTGGATGGCAGCCTGCAGATTAGCCTCCAAA
GGCAAGACCATGGCGGACAGTTCTTACAACTTAGAAGTTCAGAATATTCT
TTCCTTTCTGAAGATGCAGCATTTAAACCCAGATCCTCAGTTAATACCAG
AGCAGATCACGACTGATATAACTCCTGAATGTTTGGTGTCTCCCCGCTAT
CTAAAAAAGTATAAGAACAAGCAGATAACAGCGAGAATCTTGGAGGCCCA
TCAGAATGTAGCTCAGATGAGTCTAATTGAAGCCAAGATGAGATTTATTC
AAGCTTGGCAGTCACTACCTGAATTTGGCATCACTCACTTCATTGCAAGG
TTCCAAGGGGGCAAAAAAGAAGAACTTATTGGAATTGCATACAACAGACT
GATTCGGATGGATGCCAGCACTGGAGATGCAATTAAAACATGGCGTTTCA
GCAACATGAAACAGTGGAATGTCAACTGGGAAATCAAAATGGTCACCGTA
GAGTTTGCAGATGAAGTACGATTGTCCTTCATTTGTACTGAAGTAGATTG
CAAAGTGGTTCATGAATTCATTGGTGGCTACATATTTCTCTCAACACGTG
CAAAAGACCAAAACGAGAGTTTAGATGAAGAGATGTTCTACAAACTTACC
AGTGGTTGGGTGTGAATAGAAATACTGTTTAATGAAACTCCACGGCCATA
ACAATATTTAACTTTAAAAGCTGTTTGTTATATGCTGCTTAATAAAGTAA
GCTTGAAATTTATCATTTTATCATGAAAACTTCTTTGCCTTACCAGACCA
GTTAATATGTGCACTAAACAAGCACGACTATTAATCTATCATGTTATGAT
ATAATAAACTTGAATTTGGCACACATTCCTTAGGGCCATGAATTGAAAAC
TGAAATAGTGGGCAAATCAGGAACAAACCATCACTGATTTACTGATTTAA
GCTAGCCAAACTGTAAGAAACAAGCCATCTATTTTAAAGCTATCCAGGGC
TTAACCTATATGAACTCTATTTATCATGTCTAATGCATGTGATTTAATGT
ATGTTTAATTTGATATCATGTTTTAAAATATCCTACTTCTGGTAGCCATT
TAATTCCTCCCCCTACCCCCAAATAAATCAGGCATGCAGGAGGCCTGATA
TTTAGTAATGTCATTGTGTTTGACCTTGAAGGAAAATGCTATTAGTCCGT
CGTGCTTNATTTGTTTTTGTCCTTGAATAAGCATGTTATGTATATNGTCT
CGTGTTTTTATTTTTACACCATATTGTATTACACTTTTAGTATTCACCAG
CATAANCACTGTCTGCCTAAAATATGCAACTCTTTGCATTACAATATGAA
GTAAAGTTCTATGAAGTATGCATTTTGTGTAACTAATGTAAAAACACAAA
TTTTATAAAATTGTACAGTTTTTTAAAAACTACTCACAACTAGCAGATGG
CTTAAATGTAGCAATCTCTGCGTTAATTAAATGCCTTTAAGAGATATAAT
TAACGTGCAGTTTTAATATCTACTAAATTAAGAATGACTTCATTATGATC
ATGATTTGCCACAATGTCCTTAACTCTAATGCCTGGACTGGCCATGTTCT
AGTCTGTTGCGCTGTTACAATCTGTATTGGTGCTAGTCAGAAAATTCCTA
GCTCACATAGCCCAAAAGGGTGCGAGGGAGAGGTGGATTACCAGTATTGT
TCAATAATCCATGGTTCAAAGACTGTATAAATGCATTTTATTTTAAATAA
AAGCAAAACTTTTATTTAAA

As used herein, the term “FTH1” refers to the gene encoding Ferritin heavy chain. The terms “FTH1” and “Ferritin heavy chain” include wild-type forms of the FTH1 gene, as well as variants (e.g., splice variants and polymorphisms) of wild-type FTH1. Examples of such variants are nucleic acids having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to a wild-type FTH1 nucleic acid sequence (e.g., SEQ ID NO: 31, ENA accession number X00318). SEQ ID NO: 31 is a wild-type gene sequence encoding FTH1 protein, and is shown below:

(SEQ ID NO: 31)
CACCGCACCCTCGGACTGCCCCAAGGCCCCCGCCGCCGCTCCAGCGCCGC
GCAGCCACCGCCGCCGCCGCCGCCTCTCCTTAGTCGCCGCCATGACGACC
GCGTCCACCTCGCAGGTGCGCCAGAACTACCACCAGGACTCAGAGGCCGC
CATCAACCGCCAGATCAACCTGGAGCTCTACGCCTCCTACGTTTACCTGT
CCATGTCTTACTACTTTGACCGCGATGATGTGGCTTTGAAGAACTTTGCC
AAATACTTTCTTCACCAATCTCATGAGGAGAGGGAACATGCTGAGAAACT
GATGAAGCTGCAGAACCAACGAGGTGGCCGAATCTTCCTTCAGGATATCA
AGAAACCAGACTGTGATGACTGGGAGAGCGGGCTGAATGCAATGGAGTGT
GCATTACATTTGGAAAAAAATGTGAATCAGTCACTACTGGAACTGCACAA
ACTGGCCACTGACAAAAATGACCCCCATTTGTGTGACTTCATTGAGACAC
ATTACCTGAATGAGCAGGTGAAAGCCATCAAAGAATTGGGTGACCACGTG
ACCAACTTGCGCAAGATGGGAGCGCCCGAATCTGGCTTGGCGGAATATCT
CTTTGACAAGCACACCTGGGAGACAGTGATAATGAAAGCTAAGCCTCGGG
CTAATTTCCCATAGCCGTGGGGTGACTTCCTGGTCACCAAGGCAGTGCAT
GCATGTTGGGGTTTCCTTTACCTTTTCTATAAGTTGTACCAAAACATCCA
CTTAAGTTCTTTGATTTGTACCATTCCTTCAAATAAAGAAATTTGGTACC
C

As used herein, the term “GNAS” refers to the gene encoding Guanine nucleotide-binding protein G(s) subunit alpha isoforms XLas. The terms “GNAS” and “Guanine nucleotide-binding protein G(s) subunit alpha isoforms XLas” include wild-type forms of the GNAS gene, as well as variants (e.g., splice variants and polymorphisms) of wild-type GNAS. Examples of such variants are nucleic acids having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to a wild-type GNAS nucleic acid sequence (e.g., SEQ ID NO: 32, ENA accession number X04408). SEQ ID NO: 32 is a wild-type gene sequence encoding GNAS protein, and is shown below:

(SEQ ID NO: 32)
GCGGGCGTGCTGCCGCCGCTGCCGCCGCCGCCGCAGCCCGGCCGCGCCCC
GCCGCCGCCGCCGCCGCCATGGGCTGCCTCGGGAACAGTAAGACCGAGGA
CCAGCGCAACGAGGAGAAGGCGCAGCGTGAGGCCAACAAAAAGATCGAGA
AGCAGCTGCAGAAGGACAAGCAGGTCTACCGGGCCACGCACCGCCTGCTG
CTGCTGGGTGCTGGAGAATCTGGTAAAAGCACCATTGTGAAGCAGATGAG
GATCCTGCATGTTAATGGGTTTAATGGAGAGGGGGGCGAAGAGGACCCGC
AGGCTGCAAGGAGCAACAGCGATGGTGAGAAGGCAACCAAAGTGCAGGAC
ATCAAAAACAACCTGAAAGAGGCGATTGAAACCATTGTGGCCGCCATGAG
CAACCTGGTGCCCCCCGTGGAGCTGGCCAACCCCGAGAACCAGTTCAGAG
TGGACTACATCCTGAGTGTGATGAACGTGCCTGACTTTGACTTCCCTCCC
GAATTCTATGAGCATGCCAAGGCTCTGTGGGAGGATGAAGGAGTGCGTGC
CTGCTACGAACGCTCCAACGAGTACCAGCTGATTGACTGTGCCCAGTACT
TCCTGGACAAGATCGACGTGATCAAGCAGGCTGACTATGTGCCGAGCGAT
CAGGACCTGCTTCGCTGCCGTGTCCTGACTTCTGGAATCTTTGAGACCAA
GTTCCAGGTGGACAAAGTCAACTTCCACATGTTTGACGTGGGTGGCCAGC
GCGATGAACGCCGCAAGTGGATCCAGTGCTTCAACGATGTGACTGCCATC
ATCTTCGTGGTGGCCAGCAGCAGCTACAACATGGTCATCCGGGAGGACAA
CCAGACCAACCGCCTGCAGGAGGCTCTGAACCTCTTCAAGAGCATCTGGA
ACAACAGATGGCTGCGCACCATCTCTGTGATCCTGTTCCTCAACAAGCAA
GATCTGCTCGCTGAGAAAGTCCTTGCTGGGAAATCGAAGATTGAGGACTA
CTTTCCAGAATTTGCTCGCTACACTACTCCTGAGGATGCTACTCCCGAGC
CCGGAGAGGACCCACGCGTGACCCGGGCCAAGTACTTCATTCGAGATGAG
TTTCTGAGGATCAGCACTGCCAGTGGAGATGGGCGTCACTACTGCTACCC
TCATTTCACCTGCGCTGTGGACACTGAGAACATCCGCCGTGTGTTCAACG
ACTGCCGTGACATCATTCAGCGCATGCACCTTCGTCAGTACGAGCTGCTC
TAAGAAGGGAACCCCCAAATTTAATTAAAGCCTTAAGCACAATTAATTAA
AAGTGAAACGTAATTGTACAAGCAGTTAATCACCCACCATAGGGCATGAT
TAACAAAGCAACCTTTCCCTTCCCCCGAGTGATTTTGCGAAACCCCCTTT
TCCCTTCAGCTTGCTTAGATGTTCCAAATTTAGAAAGCTTAAGGCGGCCT
ACAGAAAAAGGAAAAAAGGCCACAAAAGTTCCCTCTCACTTTCAGTAAAA
ATAAATAAAACAGCAGCAGCAAACAAATAAAATGAAATAAAAGAAACAAA
TGAAATAAATATTGTGTTGTGCAGCATTAAAAAAAATCAAAATAAAAATT
AAATGTGAGCAAAG

As used herein, the term “GRN” refers to the gene encoding Progranulin. The terms “GRN” and “Progranulin” include wild-type forms of the GRN gene, as well as variants (e.g., splice variants and polymorphisms) of wild-type GRN. Examples of such variants are nucleic acids having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to a wild-type GRN nucleic acid sequence (e.g., SEQ ID NO: 33, ENA accession number X62320). SEQ ID NO: 33 is a wild-type gene sequence encoding GRN protein, and is shown below:

(SEQ ID NO: 33)
GCTGCTGCCCAAGGACCGCGGAGTCGGACGCAGGCAGACCATGTGGACCC
TGGTGAGCTGGGTGGCCTTAACAGCAGGGCTGGTGGCTGGAACGCGGTGC
CCAGATGGTCAGTTCTGCCCTGTGGCCTGCTGCCTGGACCCCGGAGGAGC
CAGCTACAGCTGCTGCCGTCCCCTTCTGGACAAATGGCCCACAACACTGA
GCAGGCATCTGGGTGGCCCCTGCCAGGTTGATGCCCACTGCTCTGCCGGC
CACTCCTGCATCTTTACCGTCTCAGGGACTTCCAGTTGCTGCCCCTTCCC
AGAGGCCGTGGCATGCGGGGATGGCCATCACTGCTGCCCACGGGGCTTCC
ACTGCAGTGCAGACGGGCGATCCTGCTTCCAAAGATCAGGTAACAACTCC
GTGGGTGCCATCCAGTGCCCTGATAGTCAGTTCGAATGCCCGGACTTCTC
CACGTGCTGTGTTATGGTCGATGGCTCCTGGGGGTGCTGCCCCATGCCCC
AGGCTTCCTGCTGTGAAGACAGGGTGCACTGCTGTCCGCACGGTGCCTTC
TGCGACCTGGTTCACACCCGCTGCATCACACCCACGGGCACCCACCCCCT
GGCAAAGAAGCTCCCTGCCCAGAGGACTAACAGGGCAGTGGCCTTGTCCA
GCTCGGTCATGTGTCCGGACGCACGGTCCCGGTGCCCTGATGGTTCTACC
TGCTGTGAGCTGCCCAGTGGGAAGTATGGCTGCTGCCCAATGCCCAACGC
CACCTGCTGCTCCGATCACCTGCACTGCTGCCCCCAAGACACTGTGTGTG
ACCTGATCCAGAGTAAGTGCCTCTCCAAGGAGAACGCTACCACGGACCTC
CTCACTAAGCTGCCTGCGCACACAGTGGGGGATGTGAAATGTGACATGGA
GGTGAGCTGCCCAGATGGCTATACCTGCTGCCGTCTACAGTCGGGGGCCT
GGGGCTGCTGCCCTTTTACCCAGGCTGTGTGCTGTGAGGACCACATACAC
TGCTGTCCCGCGGGGTTTACGTGTGACACGCAGAAGGGTACCTGTGAACA
GGGGCCCCACCAGGTGCCCTGGATGGAGAAGGCCCCAGCTCACCTCAGCC
TGCCAGACCCACAAGCCTTGAAGAGAGATGTCCCCTGTGATAATGTCAGC
AGCTGTCCCTCCTCCGATACCTGCTGCCAACTCACGTCTGGGGAGTGGGG
CTGCTGTCCAATCCCAGAGGCTGTCTGCTGCTCGGACCACCAGCACTGCT
GCCCCCAGGGCTACACGTGTGTAGCTGAGGGGCAGTGTCAGCGAGGAAGC
GAGATCGTGGCTGGACTGGAGAAGATGCCTGCCCGCCGGGCTTCCTTATC
CCACCCCAGAGACATCGGCTGTGACCAGCACACCAGCTGCCCGGTGGGGC
AGACCTGCTGCCCGAGCCTGGGTGGGAGCTGGGCCTGCTGCCAGTTGCCC
CATGCTGTGTGCTGCGAGGATCGCCAGCACTGCTGCCCGGCTGGCTACAC
CTGCAACGTGAAGGCTCGATCCTGCGAGAAGGAAGTGGTCTCTGCCCAGC
CTGCCACCTTCCTGGCCCGTAGCCCTCACGTGGGTGTGAAGGACGTGGAG
TGTGGGGAAGGACACTTCTGCCATGATAACCAGACCTGCTGCCGAGACAA
CCGACAGGGCTGGGCCTGCTGTCCCTACCGCCAGGGCGTCTGTTGTGCTG
ATCGGCGCCACTGCTGTCCTGCTGGCTTCCGCTGCGCAGCCAGGGGTACC
AAGTGTTTGCGCAGGGAGGCCCCGCGCTGGGACGCCCCTTTGAGGGACCC
AGCCTTGAGACAGCTGCTGTGAGGGACAGTACTGAAGACTCTGCAGCCCT
CGGGACCCCACTCGGAGGGTGCCCTCTGCTCAGGCCTCCCTAGCACCTCC
CCCTAACCAAATTCTCCCTGGACCCCATTCTGAGCTCCCCATCACCATGG
GAGGTGGGGCCTCAATCTAAGGCCTTCCCTGTCAGAAGGGGGTTGTGGCA
AAAGCCACATTACAAGCTGCCATCCCCTCCCCGTTTCAGTGGACCCTGTG
GCCAGGTGCTTTTCCCTATCCACAGGGGTGTTTGTGTGTGTGCGCGTGTG
CGTTTCAATAAAGTTTGTACACTTTCAAAAAAAAAAAAAAAAAAAAAAAA
AA

As used herein, the term “HBEGF” refers to the gene encoding Heparin Binding EGF Like Growth Factor. The terms “HBEGF” and “Heparin Binding EGF Like Growth Factor” include wild-type forms of the HBEGF gene, as well as variants (e.g., splice variants and polymorphisms) of wild-type HBEGF. Examples of such variants are nucleic acids having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to a wild-type HBEGF nucleic acid sequence (e.g., SEQ ID NO: 34, NCBI Reference Sequence: NM_001945.2). SEQ ID NO: 34 is a wild-type gene sequence encoding HBEGF protein, and is shown below:

(SEQ ID NO: 34)
ATTCGGCCGAAGGAGCTACGCGGGCCACGCTGCTGGCTGGCCTGACCTAG
GCGCGCGGGGTCGGGCGGCCGCGCGGGCGGGCTGAGTGAGCAAGACAAGA
CACTCAAGAAGAGCGAGCTGCGCCTGGGTCCCGGCCAGGCTTGCACGCAG
AGGCGGGGGGCAGACGGTGCCCGGCGGAATCTCCTGAGCTCCGCCGCCCA
GCTCTGGTGCCAGCGCCCAGTGGCCGCCGCTTCGAAAGTGACTGGTGCCT
CGCCGCCTCCTCTCGGTGCGGGACCATGAAGCTGCTGCCGTCGGTGGTGC
TGAAGCTCTTTCTGGCTGCAGTTCTCTCGGCACTGGTGACTGGCGAGAGC
CTGGAGCGGCTTCGGAGAGGGCTAGCTGCTGGAACCAGCAACCCGGACCC
TCCCACTGTATCCACGGACCAGCTGCTACCCCTAGGAGGCGGCCGGGACC
GGAAAGTCCGTGACTTGCAAGAGGCAGATCTGGACCTTTTGAGAGTCACT
TTATCCTCCAAGCCACAAGCACTGGCCACACCAAACAAGGAGGAGCACGG
GAAAAGAAAGAAGAAAGGCAAGGGGCTAGGGAAGAAGAGGGACCCATGTC
TTCGGAAATACAAGGACTTCTGCATCCATGGAGAATGCAAATATGTGAAG
GAGCTCCGGGCTCCCTCCTGCATCTGCCACCCGGGTTACCATGGAGAGAG
GTGTCATGGGCTGAGCCTCCCAGTGGAAAATCGCTTATATACCTATGACC
ACACAACCATCCTGGCCGTGGTGGCTGTGGTGCTGTCATCTGTCTGTCTG
CTGGTCATCGTGGGGCTTCTCATGTTTAGGTACCATAGGAGAGGAGGTTA
TGATGTGGAAAATGAAGAGAAAGTGAAGTTGGGCATGACTAATTCCCACT
GAGAGAGACTTGTGCTCAAGGAATCGGCTGGGGACTGCTACCTCTGAGAA
GACACAAGGTGATTTCAGACTGCAGAGGGGAAAGACTTCCATCTAGTCAC
AAAGACTCCTTCGTCCCCAGTTGCCGTCTAGGATTGGGCCTCCCATAATT
GCTTTGCCAAAATACCAGAGCCTTCAAGTGCCAAACAGAGTATGTCCGAT
GGTATCTGGGTAAGAAGAAAGCAAAAGCAAGGGACCTTCATGCCCTTCTG
ATTCCCCTCCACCAAACCCCACTTCCCCTCATAAGTTTGTTTAAACACTT
ATCTTCTGGATTAGAATGCCGGTTAAATTCCATATGCTCCAGGATCTTTG
ACTGAAAAAAAAAAAGAAGAAGAAGAAGGAGAGCAAGAAGGAAAGATTTG
TGAACTGGAAGAAAGCAACAAAGATTGAGAAGCCATGTACTCAAGTACCA
CCAAGGGATCTGCCATTGGGACCCTCCAGTGCTGGATTTGATGAGTTAAC
TGTGAAATACCACAAGCCTGAGAACTGAATTTTGGGACTTCTACCCAGAT
GGAAAAATAACAACTATTTTTGTTGTTGTTGTTTGTAAATGCCTCTTAAA
TTATATATTTATTTTATTCTATGTATGTTAATTTATTTAGTTTTTAACAA
TCTAACAATAATATTTCAAGTGCCTAGACTGTTACTTTGGCAATTTCCTG
GCCCTCCACTCCTCATCCCCACAATCTGGCTTAGTGCCACCCACCTTTGC
CACAAAGCTAGGATGGTTCTGTGACCCATCTGTAGTAATTTATTGTCTGT
CTACATTTCTGCAGATCTTCCGTGGTCAGAGTGCCACTGCGGGAGCTCTG
TATGGTCAGGATGTAGGGGTTAACTTGGTCAGAGCCACTCTATGAGTTGG
ACTTCAGTCTTGCCTAGGCGATTTTGTCTACCATTTGTGTTTTGAAAGCC
CAAGGTGCTGATGTCAAAGTGTAACAGATATCAGTGTCTCCCCGTGTCCT
CTCCCTGCCAAGTCTCAGAAGAGGTTGGGCTTCCATGCCTGTAGCTTTCC
TGGTCCCTCACCCCCATGGCCCCAGGCCCACAGCGTGGGAACTCACTTTC
CCTTGTGTCAAGACATTTCTCTAACTCCTGCCATTCTTCTGGTGCTACTC
CATGCAGGGGTCAGTGCAGCAGAGGACAGTCTGGAGAAGGTATTAGCAAA
GCAAAAGGCTGAGAAGGAACAGGGAACATTGGAGCTGACTGTTCTTGGTA
ACTGATTACCTGCCAATTGCTACCGAGAAGGTTGGAGGTGGGGAAGGCTT
TGTATAATCCCACCCACCTCACCAAAACGATGAAGTTATGCTGTCATGGT
CCTTTCTGGAAGTTTCTGGTGCCATTTCTGAACTGTTACAACTTGTATTT
CCAAACCTGGTTCATATTTATACTTTGCAATCCAAATAAAGATAACCCTT
ATTCCATAAAAAAAAAAAAAAAAAAAAAAAA

As used herein, the term “HLA-DRB1” refers to the gene encoding HLA class II histocompatibility antigen, DRB1 beta chain. The terms “HLA-DRB1” and “HLA class II histocompatibility antigen, DRB1 beta chain” include wild-type forms of the HLA-DRB1 gene, as well as variants (e.g., splice variants and polymorphisms) of wild-type HLA-DRB1. Examples of such variants are nucleic acids having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to a wild-type HLA-DRB1 nucleic acid sequence (e.g., SEQ ID NO: 35, ENA accession number X00699). SEQ ID NO: 35 is a wild-type gene sequence encoding HLA-DRB1 protein, and is shown below:

(SEQ ID NO: 35)
CTGCTCTGGCCCCTGGTCCTGTCCTGTTCTCCAGCATGGTGTGTCTGAGG
CTCCCTGGAGGCTCCTGCATGGCAGTTCTGACAGTGACACTGATGGTGCT
GAGCTCCCCACTGGCTTTGGCTGGGGACACCAGACCACGTTTCTTGGAGT
ACTCTACGTCTGAGTGTCATTTCTTCAATGGGACGGAGCGGGTGCGGTAC
CTGGACAGATACTTCCATAACCAGGAGGAGAACGTGCGCTTCGACAGCGA
CGTGGGGGAGTTCCGGGCGGTGACGGAGCTGGGGCGGCCTGATGCCGAGT
ACTGGAACAGCCAGAAGGACCTCCTGGAGCAGAAGCGGGGCCGGGTGGAC
AACTACTGCAGACACAACTACGGGGTTGTGGAGAGCTTCACAGTGCAGCG
GCGAGTCCATCCTAAGGTGACTGTGTATCCTTCAAAGACCCAGCCCCTGC
AGCACCATAACCTCCTGGTCTGTTCTGTGAGTGGTTTCTATCCAGGCAGC
ATTGAAGTCAGGTGGTTCCGGAATGGCCAGGAAGAGAAGACTGGGGTGGT
GTCCACAGGCCTGATCCACAATGGAGACTGGACCTTCCAGACCCTGGTGA
TGCTGGAAACAGTTCCTCGGAGTGGAGAGGTTTACACCTGCCAAGTGGAG
CACCCAAGCGTGACAAGCCCTCTCACAGTGGAATGGAGAGCACGGTCTGA
ATCTGCACAGAGCAAGATGCTGAGTGGAGTCGGGGGCTTTGTGCTGGGCC
TGCTCTTCCTTGGGGCCGGGCTGTTCATCTACTTCAGGAATCAGAAAGGA
CACTCTGGACTTCAGCCAAGAGGATTCCTGAGCTGAAGTGCAGATGACAC
ATTCAAAGAAGAACTTTCTGCCCCAGCTTTGCAGGATGAAAAGCTTTCCC
TCCTGGCTGTTATTCTTCCACAAGAGAGGGCTTTCTCAGGACCTGGTTGC
TACTGGTTCAGCAACTGCAGAAAATGTCCTCCCTTGTGGCTTCCTCAGCT
CCTGTTCTTGGCCTGAAGCCCCACAGCTTTGATGGCAGTGCCTCATCTTC
AACTTTTGTGCTCCCCTTTGCCTAAACCCTATGGCCTCCTGTGCATCTGT
ACTCACCCTGTACCA

As used herein, the term “HLA-DRB5” refers to the gene encoding HLA class II histocompatibility antigen, DR beta 5 chain. The terms “HLA-DRB5” and “HLA class II histocompatibility antigen, DR beta 5 chain” include wild-type forms of the HLA-DRB5 gene, as well as variants (e.g., splice variants and polymorphisms) of wild-type HLA-DRB5. Examples of such variants are nucleic acids having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to a wild-type HLA-DRB5 nucleic acid sequence (e.g., SEQ ID NO: 36, ENA accession number M20429). SEQ ID NO: 36 is a wild-type gene sequence encoding HLA-DRB5 protein, and is shown below:

(SEQ ID NO: 36)
CCAGCATGGTGTGTCTGAAGCTCCCTGGAGGTTCCTACATGGCAAAGCTGACAGTGACAC
TGATGGTGCTGAGCTCCCCACTGGCTTTGGCTGGGGACACCCGACCACGTTTCTTGCAGC
AGGATAAGTATGAGTGTCATTTCTTCAACGGGACGGAGCGGGTGCGGTTCCTGCACAGAG
ACATCTATAACCAAGAGGAGGACTTGCGCTTCGACAGCGACGTGGGGGAGTACCGGGCGG
TGACGGAGCTGGGGCGGCCTGACGCTGAGTACTGGAACAGCCAGAAGGACTTCCTGGAAG
ACAGGCGCGCCGCGGTGGACACCTACTGCAGACACAACTACGGGGTTGGTGAGAGCTTCA
CAGTGCAGCGGCGAGTTGAGCCTAAGGTGACTGTGTATCCTGCAAGGACCCAGACCCTGC
AGCACCACAACCTCCTGGTCTGCTCTGTGAATGGTTTCTATCCAGGCAGCATTGAAGTCA
GGTGGTTCCGGAACAGCCAGGAAGAGAAGGCTGGGGTGGTGTCCACAGGCCTGATTCAGA
ATGGAGACTGGACCTTCCAGACCCTGGTGATGCTGGAAACAGTTCCTCGAAGTGGAGAGG
TTTACACCTGCCAAGTGGAGCACCCAAGCGTGACGAGCCCTCTCACAGTGGAATGGAGAG
CACAGTCTGAATCTGCACAGAGCAAGATGCTGAGTGGAGTCGGGGGCTTTGTGCTGGGCC
TGCTCTTCCTTGGGGCCGGGCTATTCATCTACTTCAAGAATCAGAAAGGGCACTCTGGAC
TTCACCCAACAGGACTCGTGAGCTGAAGTGCAGATGACCACATTCAAGGGGGAACCTTCT
GCCCCAGCTTTGCATGATGAAAAGCTTTCCTGCTTGGCTCTTATTCTTCCACAAGAGAGG
ACTTTCTCAGGCCCTGGTTGCTACCGGTTCAGCAACTCTGCAGAAAATGTCCATCCTTGT
GGCTTCCTCAGCTCCTGCCCCTTGGCCTGAAGTCCCAGCATTGATGGCAGTGCCTCATCT
TCAACTTTAGTGCTCCCCTTTACCTAACCCTACGGCCTCCCATGCATCTGTACTCCCCCT
GTGTGCCACAAATGCACTACGTTATTAAATTTTTCTGAAGCCCAGAGTTAAAAATCATCT
GTCCACCTGGCTCCAAAGACAAAAAATAAAAA

As used herein, the term “IFIT1” refers to the gene encoding Interferon-induced protein with tetratricopeptide repeats 1. The terms “IFIT1” and “Interferon-induced protein with tetratricopeptide repeats 1” include wild-type forms of the IFIT1 gene, as well as variants (e.g., splice variants and polymorphisms) of wild-type IFIT1. Examples of such variants are nucleic acids having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to a wild-type IFIT1 nucleic acid sequence (e.g., SEQ ID NO: 37, ENA accession number X03557). SEQ ID NO: 37 is a wild-type gene sequence encoding IFIT1 protein, and is shown below:

(SEQ ID NO: 37)
CCAGATCTCAGAGGAGCCTGGCTAAGCAAAACCCTGCAGAACGGCTGCCTAATTTACAGC
AACCATGAGTACAAATGGTGATGATCATCAGGTCAAGGATAGTCTGGAGCAATTGAGATG
TCACTTTACATGGGAGTTATCCATTGATGACGATGAAATGCCTGATTTAGAAAACAGAGT
CTTGGATCAGATTGAATTCCTAGACACCAAATACAGTGTGGGAATACACAACCTACTAGC
CTATGTGAAACACCTGAAAGGCCAGAATGAGGAAGCCCTGAAGAGCTTAAAAGAAGCTGA
AAACTTAATGCAGGAAGAACATGACAACCAAGCAAATGTGAGGAGTCTGGTGACCTGGGG
CAACTTTGCCTGGATGTATTACCACATGGGCAGACTGGCAGAAGCCCAGACTTACCTGGA
CAAGGTGGAGAACATTTGCAAGAAGCTTTCAAATCCCTTCCGCTATAGAATGGAGTGTCC
AGAAATAGACTGTGAGGAAGGATGGGCCTTGCTGAAGTGTGGAGGAAAGAATTATGAACG
GGCCAAGGCCTGCTTTGAAAAGGTGCTTGAAGTGGACCCTGAAAACCCTGAATCCAGCGC
TGGGTATGCGATCTCTGCCTATCGCCTGGATGGCTTTAAATTAGCCACAAAAAATCACAA
GCCATTTTCTTTGCTTCCCCTAAGGCAGGCTGTCCGCTTAAATCCAGACAATGGATATAT
TAAGGTTCTCCTTGCCCTGAAGCTTCAGGATGAAGGACAGGAAGCTGAAGGAGAAAAGTA
CATTGAAGAAGCTCTAGCCAACATGTCCTCACAGACCTATGTCTTTCGATATGCAGCCAA
GTTTTACCGAAGAAAAGGCTCTGTGGATAAAGCTCTTGAGTTATTAAAAAAGGCCTTGCA
GGAAACACCCACTTCTGTCTTACTGCATCACCAGATAGGGCTTTGCTACAAGGCACAAAT
GATCCAAATCAAGGAGGCTACAAAAGGGCAGCCTAGAGGGCAGAACAGAGAAAAGCTAGA
CAAAATGATAAGATCAGCCATATTTCATTTTGAATCTGCAGTGGAAAAAAAGCCCACATT
TGAGGTGGCTCATCTAGACCTGGCAAGAATGTATATAGAAGCAGGCAATCACAGAAAAGC
TGAAGAGAATTTTCAAAAATTGTTATGCATGAAACCAGTGGTAGAAGAAACAATGCAAGA
CATACATTTCTACTATGGTCGGTTTCAGGAATTTCAAAAGAAATCTGACGTCAATGCAAT
TATCCATTATTTAAAAGCTATAAAAATAGAACAGGCATCATTAACAAGGGATAAAAGTAT
CAATTCTTTGAAGAAATTGGTTTTAAGGAAACTTCGGAGAAAGGCATTAGATCTGGAAAG
CTTGAGCCTCCTTGGGTTCGTCTACAAATTGGAAGGAAATATGAATGAAGCCCTGGAGTA
CTATGAGCGGGCCCTGAGACTGGCTGCTGACTTTGAGAACTCTGTGAGACAAGGTCCTTA
GGCACCCAGATATCAGCCACTTTCACATTTCATTTCATTTTATGCTAACATTTACTAATC
ATCTTTTCTGCTTACTGTTTTCAGAAACATTATAATTCACTGTAATGATGTAATTCTTGA
ATAATAAATCTGACAAAATATT

As used herein, the term “IFIT3” refers to the gene encoding Interferon-induced protein with tetratricopeptide repeats 3. The terms “IFIT3” and “Interferon-induced protein with tetratricopeptide repeats 3” include wild-type forms of the IFIT3 gene, as well as variants (e.g., splice variants and polymorphisms) of wild-type IFIT3. Examples of such variants are nucleic acids having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to a wild-type IFIT3 nucleic acid sequence (e.g., SEQ ID NO: 38, ENA accession number AF026939). SEQ ID NO: 38 is a wild-type gene sequence encoding IFIT3 protein, and is shown below:

(SEQ ID NO: 38)
GTGGAAACCTCTTCAGCATTTGCTTGGAATCAGTAAGCTAAAAACAAAATCAACCGGGAC
CCCAGCTTTTCAGAACTGCAGGGAAACAGCCATCATGAGTGAGGTCACCAAGAATTCCCT
GGAGAAAATCCTCCCACAGCTGAAATGCCATTTCACCTGGAACTTATTCAAGGAAGACAG
TGTCTCAAGGGATCTAGAAGATAGAGTGTGTAACCAGATTGAATTTTTAAACACTGAGTT
CAAAGCTACAATGTACAACTTGTTGGCCTACATAAAACACCTAGATGGTAACAACGAGGC
AGCCCTGGAATGCTTACGGCAAGCTGAAGAGTTAATCCAGCAAGAACATGCTGACCAAGC
AGAAATCAGAAGTCTAGTCACTTGGGGAAACTACGCCTGGGTCTACTATCACTTGGGCAG
ACTCTCAGATGCTCAGATTTATGTAGATAAGGTGAAACAAACCTGCAAGAAATTTTCAAA
TCCATACAGTATTGAGTATTCTGAACTTGACTGTGAGGAAGGGTGGACACAACTGAAGTG
TGGAAGAAATGAAAGGGCGAAGGTGTGTTTTGAGAAGGCTCTGGAAGAAAAGCCCAACAA
CCCAGAATTCTCCTCTGGACTGGCAATTGCGATGTACCATCTGGATAATCACCCAGAGAA
ACAGTTCTCTACTGATGTTTTGAAGCAGGCCATTGAGCTGAGTCCTGATAACCAATACGT
CAAGGTTCTCTTGGGCCTGAAACTGCAGAAGATGAATAAAGAAGCTGAAGGAGAGCAGTT
TGTTGAAGAAGCCTTGGAAAAGTCTCCTTGCCAAACAGATGTCCTCCGCAGTGCAGCCAA
ATTTTACAGAAGAAAAGGTGACCTAGACAAAGCTATTGAACTGTTTCAACGGGTGTTGGA
ATCCACACCAAACAATGGCTACCTCTATCACCAGATTGGGTGCTGCTACAAGGCAAAAGT
AAGACAAATGCAGAATACAGGAGAATCTGAAGCTAGTGGAAATAAAGAGATGATTGAAGC
ACTAAAGCAATATGCTATGGACTATTCGAATAAAGCTCTTGAGAAGGGACTGAATCCTCT
GAATGCATACTCCGATCTCGCTGAGTTCCTGGAGACGGAATGTTATCAGACACCATTCAA
TAAGGAAGTCCCTGATGCTGAAAAGCAACAATCCCATCAGCGCTACTGCAACCTTCAGAA
ATATAATGGGAAGTCTGAAGACACTGCTGTGCAACATGGTTTAGAGGGTTTGTCCATAAG
CAAAAAATCAACTGACAAGGAAGAGATCAAAGACCAACCACAGAATGTATCCGAAAATCT
GCTTCCACAAAATGCACCAAATTATTGGTATCTTCAAGGATTAATTCATAAGCAGAATGG
AGATCTGCTGCAAGCAGCCAAATGTTATGAGAAGGAACTGGGCCGCCTGCTAAGGGATGC
CCCTTCAGGCATAGGCAGTATTTTCCTGTCAGCATCTGAGCTTGAGGATGGTAGTGAGGA
AATGGGCCAGGGCGCAGTCAGCTCCAGTCCCAGAGAGCTCCTCTCTAACTCAGAGCAACT
GAACTGAGACAGAGGAGGAAAACAGAGCATCAGAAGCCTGCAGTGGTGGTTGTGACGGGT
AGGAGGATAGGAAGACAGGGGGCCCCAACCTGGGATTGCTGAGCAGGGAAGCTTTGCATG
TTGCTCTAAGGTACATTTTTAAAGAGTTGTTTTTTGGCCGGGCGCAGTGGCTCATGCCTG
TAATCCCAGCACTTTGGGAGGCCGAGGTGGGCGGATCACGAGGTCTGGAGTTTGAGACCA
TCCTGGCTAACACAGTGAAATCCCGTCTCTACTAAAAATACAAAAAATTAGCCAGGCGTG
GTGGCTGGCACCTGTAGTCCCAGCTACTTGGGAGGCTGAGGCAGGAGAATGGCGTGAACC
TGGAAGGAAGAGGTTGCAGTGAGCCAAGATTGCGCCCCTGCACTCCAGCCTGGGCAACAG
AGCAAGACTC

As used herein, the term “IFITM3” refers to the gene encoding Interferon Induced Transmembrane Protein. The terms “IFITM3” and “Interferon Induced Transmembrane Protein” include wild-type forms of the IFITM3 gene, as well as variants (e.g., splice variants and polymorphisms) of wild-type IFITM3. Examples of such variants are nucleic acids having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to a wild-type IFITM3 nucleic acid sequence (e.g., SEQ ID NO: 39, NCBI Reference Sequence: NM_021034.2). SEQ ID NO: 39 is a wild-type gene sequence encoding IFITM3 protein, and is shown below:

(SEQ ID NO: 39)
AGGAAAAGGAAACTGTTGAGAAACCGAAACTACTGGGGAAAGGGAGGGCTCACTGAGAACCATCCC
AGTAACCCGACCGCCGCTGGTCTTCGCTGGACACCATGAATCACACTGTCCAAACCTTCTTCTCTCC
TGTCAACAGTGGCCAGCCCCCCAACTATGAGATGCTCAAGGAGGAGCACGAGGTGGCTGTGCTGG
GGGCGCCCCACAACCCTGCTCCCCCGACGTCCACCGTGATCCACATCCGCAGCGAGACCTCCGTG
CCCGACCATGTCGTCTGGTCCCTGTTCAACACCCTCTTCATGAACCCCTGCTGCCTGGGCTTCATAG
CATTCGCCTACTCCGTGAAGTCTAGGGACAGGAAGATGGTTGGCGACGTGACCGGGGCCCAGGCC
TATGCCTCCACCGCCAAGTGCCTGAACATCTGGGCCCTGATTCTGGGCATCCTCATGACCATTCTGC
TCATCGTCATCCCAGTGCTGATCTTCCAGGCCTATGGATAGATCAGGAGGCATCACTGAGGCCAGG
AGCTCTGCCCATGACCTGTATCCCACGTACTCCAACTTCCATTCCTCGCCCTGCCCCCGGAGCCGA
GTCCTGTATCAGCCCTTTATCCTCACACGCTTTTCTACAATGGCATTCAATAAAGTGCACGTGTTTCT
GGTGCTAAAAAAAAAA

As used herein, the term “IFNAR1” refers to the gene encoding Interferon alpha/beta receptor 1. The terms “IFNAR1” and “Interferon alpha/beta receptor 1” include wild-type forms of the IFNAR1 gene, as well as variants (e.g., splice variants and polymorphisms) of wild-type IFNAR1. Examples of such variants are nucleic acids having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to a wild-type IFNAR1 nucleic acid sequence (e.g., SEQ ID NO: 40, ENA accession number J03171). SEQ ID NO: 40 is a wild-type gene sequence encoding IFNAR1 protein, and is shown below:

(SEQ ID NO: 40)
TTAGGACGGGGCGATGGCGGCTGAGAGGAGCTGCGCGTGCGCGAACATGTAACTGGTGGG
ATCTGCGGCGGCTCCCAGATGATGGTCGTCCTCCTGGGCGCGACGACCCTAGTGCTCGTC
GCCGTGGGCCCATGGGTGTTGTCCGCAGCCGCAGGTGGAAAAAATCTAAAATCTCCTCAA
AAAGTAGAGGTCGACATCATAGATGACAACTTTATCCTGAGGTGGAACAGGAGCGATGAG
TCTGTCGGGAATGTGACTTTTTCATTCGATTATCAAAAAACTGGGATGGATAATTGGATA
AAATTGTCTGGGTGTCAGAATATTACTAGTACCAAATGCAACTTTTCTTCACTCAAGCTG
AATGTTTATGAAGAAATTAAATTGCGTATAAGAGCAGAAAAAGAAAACACTTCTTCATGG
TATGAGGTTGACTCATTTACACCATTTCGCAAAGCTCAGATTGGTCCTCCAGAAGTACAT
TTAGAAGCTGAAGATAAGGCAATAGTGATACACATCTCTCCTGGAACAAAAGATAGTGTT
ATGTGGGCTTTGGATGGTTTAAGCTTTACATATAGCTTACTTATCTGGAAAAACTCTTCA
GGTGTAGAAGAAAGGATTGAAAATATTTATTCCAGACATAAAATTTATAAACTCTCACCA
GAGACTACTTATTGTCTAAAAGTTAAAGCAGCACTACTTACGTCATGGAAAATTGGTGTC
TATAGTCCAGTACATTGTATAAAGACCACAGTTGAAAATGAACTACCTCCACCAGAAAAT
ATAGAAGTCAGTGTCCAAAATCAGAACTATGTTCTTAAATGGGATTATACATATGCAAAC
ATGACCTTTCAAGTTCAGTGGCTCCACGCCTTTTTAAAAAGGAATCCTGGAAACCATTTG
TATAAATGGAAACAAATACCTGACTGTGAAAATGTCAAAACTACCCAGTGTGTCTTTCCT
CAAAACGTTTTCCAAAAAGGAATTTACCTTCTCCGCGTACAAGCATCTGATGGAAATAAC
ACATCTTTTTGGTCTGAAGAGATAAAGTTTGATACTGAAATACAAGCTTTCCTACTTCCT
CCAGTCTTTAACATTAGATCCCTTAGTGATTCATTCCATATCTATATCGGTGCTCCAAAA
CAGTCTGGAAACACGCCTGTGATCCAGGATTATCCACTGATTTATGAAATTATTTTTTGG
GAAAACACTTCAAATGCTGAGAGAAAAATTATCGAGAAAAAAACTGATGTTACAGTTCCT
AATTTGAAACCACTGACTGTATATTGTGTGAAAGCCAGAGCACACACCATGGATGAAAAG
CTGAATAAAAGCAGTGTTTTTAGTGACGCTGTATGTGAGAAAACAAAACCAGGAAATACC
TCTAAAATTTGGCTTATAGTTGGAATTTGTATTGCATTATTTGCTCTCCCGTTTGTCATT
TATGCTGCGAAAGTCTTCTTGAGATGCATCAATTATGTCTTCTTTCCATCACTTAAACCT
TCTTCCAGTATAGATGAGTATTTCTCTGAACAGCCATTGAAGAATCTTCTGCTTTCAACT
TCTGAGGAACAAATCGAAAAATGTTTCATAATTGAAAATATAAGCACAATTGCTACAGTA
GAAGAAACTAATCAAACTGATGAAGATCATAAAAAATACAGTTCCCAAACTAGCCAAGAT
TCAGGAAATTATTCTAATGAAGATGAAAGCGAAAGTAAAACAAGTGAAGAACTACAGCAG
GACTTTGTATGACCAGAAATGAACTGTGTCAAGTATAAGGTTTTTCAGCAGGAGTTACAC
TGGGAGCCTGAGGTCCTCACCTTCCTCTCAGTAACTACAGAGAGGACGTTTCCTGTTTAG
GGAAAGAAAAAACATCTTCAGATCATAGGTCCTAAAAATACGGGCAAGCTCTTAACTATT
TAAAAATGAAATTACAGGCCCGGGCACGGTGGCTCACACCTGTAATCCCAGCACTTTGGG
AGGCTGAGGCAGGCAGATCATGAGGTCAAGAGATCGAGACCAGCCTGGCCAACGTGGTGA
AACCCCATCTCTACTAAAAATACAAAAATTAGCCGGGTAGTAGGTAGGCGCGCGCCTGTT
GTCTTAGCTACTCAGGAGGCTGAGGCAGGAGAATCGCTTGAAAACAGGAGGTGGAGGTTG
CAGTGAGCCGAGATCACGCCACTGCACTCCAGCCTGGTGACAGCGTGAGACTCTTTAAAA
AAAGAAATTAAAAGAGTTGAGACAAACGTTTCCTACATTCTTTTCCATGTGTAAAATCAT
GAAAAAGCCTGTCACCGGACTTGCATTGGATGAGATGAGTCAGACCAAAACAGTGGCCAC
CCGTCTTCCTCCTGTGAGCCTAAGTGCAGCCGTGCTAGCTGCGCACCGTGGCTAAGGATG
ACGTCTGTGTTCCTGTCCATCACTGATGCTGCTGGCTACTGCATGTGCCACACCTGTCTG
TTCGCCATTCCTAACATTCTGTTTCATTCTTCCTCGGGAGATATTTCAAACATTTGGTCT
TTTCTTTTAACACTGAGGGTAGGCCCTTAGGAAATTTATTTAGGAAAGTCTGAACACGTT
ATCACTTGGTTTTCTGGAAAGTAGCTTACCCTAGAAAACAGCTGCAAATGCCAGAAAGAT
GATCCCTAAAAATGTTGAGGGACTTCTGTTCATTCATCCCGAGAACATTGGCTTCCACAT
CACAGTATCTACCCTTACATGGTTTAGGATTAAAGCCAGGCAATCTTTTACTATG

As used herein, the term “IFNAR2” refers to the gene encoding Interferon alpha/beta receptor 2. The terms “IFNAR2” and “Interferon alpha/beta receptor 2” include wild-type forms of the IFNAR2 gene, as well as variants (e.g., splice variants and polymorphisms) of wild-type IFNAR2. Examples of such variants are nucleic acids having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to a wild-type IFNAR2 nucleic acid sequence (e.g., SEQ ID NO: 41, ENA accession number X77722). SEQ ID NO: 41 is a wild-type gene sequence encoding IFNAR2 protein, and is shown below:

(SEQ ID NO: 41)
GCTTTTGTCCCCCGCCCGCCGCTTCTGTCCGAGAGGCCGCCCGCGAGGCGCATCCTGACC
GCGAGCGTCGGGTCCCAGAGCCGGGCGCGGCTGGGGCCCGAGGCTAGCATCTCTCGGGAG
CCGCAAGGCGAGAGCTGCAAAGTTTAATTAGACACTTCAGAATTTTGATCACCTAATGTT
GATTTCAGATGTAAAAGTCAAGAGAAGACTCTAAAAATAGCAAAGATGCTTTTGAGCCAG
AATGCCTTCATCGTCAGATCACTTAATTTGGTTCTCATGGTGTATATCAGCCTCGTGTTT
GGTATTTCATATGATTCGCCTGATTACACAGATGAATCTTGCACTTTCAAGATATCATTG
CGAAATTTCCGGTCCATCTTATCATGGGAATTAAAAAACCACTCCATTGTACCAACTCAC
TATACATTGCTGTATACAATCATGAGTAAACCAGAAGATTTGAAGGTGGTTAAGAACTGT
GCAAATACCACAAGATCATTTTGTGACCTCACAGATGAGTGGAGAAGCACACACGAGGCC
TATGTCACCGTCCTAGAAGGATTCAGCGGGAACACAACGTTGTTCAGTTGCTCACACAAT
TTCTGGCTGGCCATAGACATGTCTTTTGAACCACCAGAGTTTGAGATTGTTGGTTTTACC
AACCACATTAATGTGATGGTGAAATTTCCATCTATTGTTGAGGAAGAATTACAGTTTGAT
TTATCTCTCGTCATTGAAGAACAGTCAGAGGGAATTGTTAAGAAGCATAAACCCGAAATA
AAAGGAAACATGAGTGGAAATTTCACCTATATCATTGACAAGTTAATTCCAAACACGAAC
TACTGTGTATCTGTTTATTTAGAGCACAGTGATGAGCAAGCAGTAATAAAGTCTCCCTTA
AAATGCACCCTCCTTCCACCTGGCCAGGAATCAGAATCAGCAGAATCTGCCAAAATAGGA
GGAATAATTACTGTGTTTTTGATAGCATTGGTCTTGACAAGCACCATAGTGACACTGAAA
TGGATTGGTTATATATGCTTAAGAAATAGCCTCCCCAAAGTCTTGAGGCAAGGTCTCACT
AAGGGCTGGAATGCAGTGGCTATTCACAGGTGCAGTCATAATGCACTACAGTCTGAAACT
CCTGAGCTCAAACAGTCGTCCTGCCTAAGCTTCCCCAGTAGCTGGGATTACAAGCGTGCA
TCCCTGTGCCCCAGTGATTAAGTTTTATTATGTAGAAAATAAAGAGCAAACAGTTACAAA
AGAAAAAAAAAAAAAAAAAAAAAAAAAAAAAAAAAA

As used herein, the term “IGF1” refers to the gene encoding Insulin-like growth factor I. The terms “IGF1” and “Insulin-like growth factor I” include wild-type forms of the IGF1 gene, as well as variants (e.g., splice variants and polymorphisms) of wild-type IGF1. Examples of such variants are nucleic acids having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to a wild-type IGF1 nucleic acid sequence (e.g., SEQ ID NO: 42, ENA accession number X00173). SEQ ID NO: 42 is a wild-type gene sequence encoding IGF1 protein, and is shown below:

(SEQ ID NO: 42)
CTTCAGAAGCAATGGGAAAAATCAGCAGTCTTCCAACCCAATTATTTAAGTGCTGCTTTT
GTGATTTCTTGAAGGTGAAGATGCACACCATGTCCTCCTCGCATCTCTTCTACCTGGCGC
TGTGCCTGCTCACCTTCACCAGCTCTGCCACGGCTGGACCGGAGACGCTCTGCGGGGCTG
AGCTGGTGGATGCTCTTCAGTTCGTGTGTGGAGACAGGGGCTTTTATTTCAACAAGCCCA
CAGGGTATGGCTCCAGCAGTCGGAGGGCGCCTCAGACAGGTATCGTGGATGAGTGCTGCT
TCCGGAGCTGTGATCTAAGGAGGCTGGAGATGTATTGCGCACCCCTCAAGCCTGCCAAGT
CAGCTCGCTCTGTCCGTGCCCAGCGCCACACCGACATGCCCAAGACCCAGAAGGAAGTAC
ATTTGAAGAACGCAAGTAGAGGGAGTGCAGGAAACAAGAACTACAGGATGTAGGAAGACC
CTCCTGAGGAGTGAAGAGTGACATGCCACCGCAGGATCCTTTGCTCTGCACGAGTTACCT
GTTAAACTTTGGAACACCTACCAAAAAATAAGTTTGATAACATTTAAAAGATGGGCGTTT
CCCCCAATGAAATACACAAGTAAACATTCCAACATTGTCTTTAGGAGTGATTTGCACCTT
GCAAAAATGGTCCTGGAGTTGGTAGATTGCTGTTGATCTTTTATCAATAATGTTCTATAG
AAAAG

As used herein, the term “IL10RA” refers to the gene encoding Interleukin-10 receptor subunit alpha. The terms “IL10RA” and “Interleukin-10 receptor subunit alpha” include wild-type forms of the MORA gene, as well as variants (e.g., splice variants and polymorphisms) of wild-type MORA. Examples of such variants are nucleic acids having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to a wild-type IL10RA nucleic acid sequence (e.g., SEQ ID NO: 43, ENA accession number U00672). SEQ ID NO: 43 is a wild-type gene sequence encoding MORA protein, and is shown below:

(SEQ ID NO: 43)
AAAGAGCTGGAGGCGCGCAGGCCGGCTCCGCTCCGGCCCCGGACGATGCGGCGCGCCCAG
GATGCTGCCGTGCCTCGTAGTGCTGCTGGCGGCGCTCCTCAGCCTCCGTCTTGGCTCAGA
CGCTCATGGGACAGAGCTGCCCAGCCCTCCGTCTGTGTGGTTTGAAGCAGAATTTTTCCA
CCACATCCTCCACTGGACACCCATCCCAAATCAGTCTGAAAGTACCTGCTATGAAGTGGC
GCTCCTGAGGTATGGAATAGAGTCCTGGAACTCCATCTCCAACTGTAGCCAGACCCTGTC
CTATGACCTTACCGCAGTGACCTTGGACCTGTACCACAGCAATGGCTACCGGGCCAGAGT
GCGGGCTGTGGACGGCAGCCGGCACTCCAACTGGACCGTCACCAACACCCGCTTCTCTGT
GGATGAAGTGACTCTGACAGTTGGCAGTGTGAACCTAGAGATCCACAATGGCTTCATCCT
CGGGAAGATTCAGCTACCCAGGCCCAAGATGGCCCCCGCGAATGACACATATGAAAGCAT
CTTCAGTCACTTCCGAGAGTATGAGATTGCCATTCGCAAGGTGCCGGGAAACTTCACGTT
CACACACAAGAAAGTAAAACATGAAAACTTCAGCCTCCTAACCTCTGGAGAAGTGGGAGA
GTTCTGTGTCCAGGTGAAACCATCTGTCGCTTCCCGAAGTAACAAGGGGATGTGGTCTAA
AGAGGAGTGCATCTCCCTCACCAGGCAGTATTTCACCGTGACCAACGTCATCATCTTCTT
TGCCTTTGTCCTGCTGCTCTCCGGAGCCCTCGCCTACTGCCTGGCCCTCCAGCTGTATGT
GCGGCGCCGAAAGAAGCTACCCAGTGTCCTGCTCTTCAAGAAGCCCAGCCCCTTCATCTT
CATCAGCCAGCGTCCCTCCCCAGAGACCCAAGACACCATCCACCCGCTTGATGAGGAGGC
CTTTTTGAAGGTGTCCCCAGAGCTGAAGAACTTGGACCTGCACGGCAGCACAGACAGTGG
CTTTGGCAGCACCAAGCCATCCCTGCAGACTGAAGAGCCCCAGTTCCTCCTCCCTGACCC
TCACCCCCAGGCTGACAGAACGCTGGGAAACGGGGAGCCCCCTGTGCTGGGGGACAGCTG
CAGTAGTGGCAGCAGCAATAGCACAGACAGCGGGATCTGCCTGCAGGAGCCCAGCCTGAG
CCCCAGCACAGGGCCCACCTGGGAGCAACAGGTGGGGAGCAACAGCAGGGGCCAGGATGA
CAGTGGCATTGACTTAGTTCAAAACTCTGAGGGCCGGGCTGGGGACACACAGGGTGGCTC
GGCCTTGGGCCACCACAGTCCCCCGGAGCCTGAGGTGCCTGGGGAAGAAGACCCAGCTGC
TGTGGCATTCCAGGGTTACCTGAGGCAGACCAGATGTGCTGAAGAGAAGGCAACCAAGAC
AGGCTGCCTGGAGGAAGAATCGCCCTTGACAGATGGCCTTGGCCCCAAATTCGGGAGATG
CCTGGTTGATGAGGCAGGCTTGCATCCACCAGCCCTGGCCAAGGGCTATTTGAAACAGGA
TCCTCTAGAAATGACTCTGGCTTCCTCAGGGGCCCCAACGGGACAGTGGAACCAGCCCAC
TGAGGAATGGTCACTCCTGGCCTTGAGCAGCTGCAGTGACCTGGGAATATCTGACTGGAG
CTTTGCCCATGACCTTGCCCCTCTAGGCTGTGTGGCAGCCCCAGGTGGTCTCCTGGGCAG
CTTTAACTCAGACCTGGTCACCCTGCCCCTCATCTCTAGCCTGCAGTCAAGTGAGTGACT
CGGGCTGAGAGGCTGCTTTTGATTTTAGCCATGCCTGCTCCTCTGCCTGGACCAGGAGGA
GGGCCCTGGGGCAGAAGTTAGGCACGAGGCAGTCTGGGCACTTTTCTGCAAGTCCACTGG
GGCTGGCCCAGCCAGGCTGCAGGGCTGGTCAGGGTGTCTGGGGCAGGAGGAGGCCAACTC
ACTGAACTAGTGCAGGGTATGTGGGTGGCACTGACCTGTTCTGTTGACTGGGGCCCTGCA
GACTCTGGCAGAGCTGAGAAGGGCAGGGACCTTCTCCCTCCTAGGAACTCTTTCCTGTAT
CATAAAGGATTATTTGCTCAGGGGAACCATGGGGCTTTCTGGAGTTGTGGTGAGGCCACC
AGGCTGAAGTCAGCTCAGACCCAGACCTCCCTGCTTAGGCCACTCGAGCATCAGAGCTTC
CAGCAGGAGGAAGGGCTGTAGGAATGGAAGCTTCAGGGCCTTGCTGCTGGGGTCATTTTT
AGGGGAAAAAGGAGGATATGATGGTCACATGGGGAACCTCCCCTCATCGGGCCTCTGGGG
CAGGAAGCTTGTCACTGGAAGATCTTAAGGTATATATTTTCTGGACACTCAAACACATCA
TAATGGATTCACTGAGGGGAGACAAAGGGAGCCGAGACCCTGGATGGGGCTTCCAGCTCA
GAACCCATCCCTCTGGTGGGTACCTCTGGCACCCATCTGCAAATATCTCCCTCTCTCCAA
CAAATGGAGTAGCATCCCCCTGGGGCACTTGCTGAGGCCAAGCCACTCACATCCTCACTT
TGCTGCCCCACCATCTTGCTGACAACTTCCAGAGAAGCCATGGTTTTTTGTATTGGTCAT
AACTCAGCCCTTTGGGCGGCCTCTGGGCTTGGGCACCAGCTCATGCCAGCCCCAGAGGGT
CAGGGTTGGAGGCCTGTGCTTGTGTTTGCTGCTAATGTCCAGCTACAGACCCAGAGGATA
AGCCACTGGGCACTGGGCTGGGGTCCCTGCCTTGTTGGTGTTCAGCTGTGTGATTTTGGA
CTAGCCACTTGTCAGAGGGCCTCAATCTCCCATCTGTGAAATAAGGACTCCACCTTTAGG
GGACCCTCCATGTTTGCTGGGTATTAGCCAAGCTGGTCCTGGGAGAATGCAGATACTGTC
CGTGGACTACCAAGCTGGCTTGTTTCTTATGCCAGAGGCTAACAGATCCAATGGGAGTCC
ATGGTGTCATGCCAAGACAGTATCAGACACAGCCCCAGAAGGGGGCATTATGGGCCCTGC
CTCCCCATAGGCCATTTGGACTCTGCCTTCAAACAAAGGCAGTTCAGTCCACAGGCATGG
AAGCTGTGAGGGGACAGGCCTGTGCGTGCCATCCAGAGTCATCTCAGCCCTGCCTTTCTC
TGGAGCATTCTGAAAACAGATATTCTGGCCCAGGGAATCCAGCCATGACCCCCACCCCTC
TGCCAAAGTACTCTTAGGTGCCAGTCTGGTAACTGAACTCCCTCTGGAGGCAGGCTTGAG
GGAGGATTCCTCAGGGTTCCCTTGAAAGCTTTATTTATTTATTTTGTTCATTTATTTATT
GGAGAGGCAGCATTGCACAGTGAAAGAATTCTGGATATCTCAGGAGCCCCGAAATTCTAG
CTCTGACTTTGCTGTTTCCAGTGGTATGACCTTGGAGAAGTCACTTATCCTCTTGGAGCC
TCAGTTTCCTCATCTGCAGAATAATGACTGACTTGTCTAATTCATAGGGATGTGAGGTTC
TGCTGAGGAAATGGGTATGAATGTGCCTTGAACACAAAGCTCTGTCAATAAGTGATACAT
GTTTTTTATTCCAATAAATTGTCAAGACCACA

As used herein, the term “ILIA” refers to the gene encoding Interleukin-1 alpha. The terms “ILIA” and “Interleukin-1 alpha” include wild-type forms of the ILIA gene, as well as variants (e.g., splice variants and polymorphisms) of wild-type ILIA. Examples of such variants are nucleic acids having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to a wild-type ILIA nucleic acid sequence (e.g., SEQ ID NO: 44, ENA accession number X02531). SEQ ID NO: 44 is a wild-type gene sequence encoding ILIA protein, and is shown below:

(SEQ ID NO: 44)
ATGGCCAAAGTTCCAGACATGTTTGAAGACCTGAAGAACTGTTACAGTGAAAATGAAGAA
GACAGTTCCTCCATTGATCATCTGTCTCTGAATCAGAAATCCTTCTATCATGTAAGCTAT
GGCCCACTCCATGAAGGCTGCATGGATCAATCTGTGTCTCTGAGTATCTCTGAAACCTCT
AAAACATCCAAGCTTACCTTCAAGGAGAGCATGGTGGTAGTAGCAACCAACGGGAAGGTT
CTGAAGAAGAGACGGTTGAGTTTAAGCCAATCCATCACTGATGATGACCTGGAGGCCATC
GCCAATGACTCAGAGGAAGAAATCATCAAGCCTAGGTCAGCACCTTTTAGCTTCCTGAGC
AATGTGAAATACAACTTTATGAGGATCATCAAATACGAATTCATCCTGAATGACGCCCTC
AATCAAAGTATAATTCGAGCCAATGATCAGTACCTCACGGCTGCTGCATTACATAATCTG
GATGAAGCAGTGAAATTTGACATGGGTGCTTATAAGTCATCAAAGGATGATGCTAAAATT
ACCGTGATTCTAAGAATCTCAAAAACTCAATTGTATGTGACTGCCCAAGATGAAGACCAA
CCAGTGCTGCTGAAGGAGATGCCTGAGATACCCAAAACCATCACAGGTAGTGAGACCAAC
CTCCTCTTCTTCTGGGAAACTCACGGCACTAAGAACTATTTCACATCAGTTGCCCATCCA
AACTTGTTTATTGCCACAAAGCAAGACTACTGGGTGTGCTTGGCAGGGGGGCCACCCTCT
ATCACTGACTTTCAGATACTGGAAAACCAGGCGTAGGTCTGGAGTCTCACTTGTCTCACT
TGTGCAGTGTTGACAGTTCATATGTACCATGTACATGAAGAAGCTAAATCCTTTACTGTT
AGTCATTTGCTGAGCATGTACTGAGCCTTGTAATTCTAAATGAATGTTTACACTCTTTGT
AAGAGTGGAACCAACACTAACATATAATGTTGTTATTTAAAGAACACCCTATATTTTGCA
TAGTACCAATCATTTTAATTATTATTCTTCATAACAATTTTAGGAGGACCAGAGCTACTG
ACTATGGCTACCAAAAAGACTCTACCCATATTACAGATGGGCAAATTAAGGCATAAGAAA
ACTAAGAAATATGCACAATAGCAGTTGAAACAAGAAGCCACAGACCTAGGATTTCATGAT
TTCATTTCAACTGTTTGCCTTCTGCTTTTAAGTTGCTGATGAACTCTTAATCAAATAGCA
TAAGTTTCTGGGACCTCAGTTTTATCATTTTCAAAATGGAGGGAATAATACCTAAGCCTT
CCTGCCGCAACAGTTTTTTATGCTAATCAGGGAGGTCATTTTGGTAAAATACTTCTCGAA
GCCGAGCCTCAAGATGAAGGCAAAGCACGAAATGTTATTTTTTAATTATTATTTATATAT
GTATTTATAAATATATTTAAGATAATTATAATATACTATATTTATGGGAACCCCTTCATC
CTCTGAGTGTGACCAGGCATCCTCCACAATAGCAGACAGTGTTTTCTGGGATAAGTAAGT
TTGATTTCATTAATACAGGGCATTTTGGTCCAAGTTGTGCTTATCCCATAGCCAGGAAAC
TCTGCATTCTAGTACTTGGGAGACCTGTAATCATATAATAAATGTACATTAATTACCTTG
AGCCAGTAATTGGTCCGATCTTTGACTCTTTTGCCATTAAACTTACCTGGGCATTCTTGT
TTCATTCAATTCCACCTGCAATCAAGTCCTACAAGCTAAAATTAGATGAACTCAACTTTG
ACAACCATAGACCACTGTTATCAAAACTTTCTTTTCTGGAATGTAATCAATGTTTCTTCT
AGGTTCTAAAAATTGTGATCAGACCATAATGTTACATTATTATCAACAATAGTGATTGAT
AGAGTGTTATCAGTCATAACTAAATAAAGCTTGCAAGTGAGGGAGTCATTTCATTGGCGT
TTGAGTCAGCAAAGAAGTCAAG

As used herein, the term “IL1B” refers to the gene encoding Interleukin-1 beta. The terms “IL1B” and “Interleukin-1 beta” include wild-type forms of the IL1B gene, as well as variants (e.g., splice variants and polymorphisms) of wild-type IL1B. Examples of such variants are nucleic acids having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to a wild-type IL1B nucleic acid sequence (e.g., SEQ ID NO: 45, ENA accession number X02770). SEQ ID NO: 45 is a wild-type gene sequence encoding IL1B protein, and is shown below:

(SEQ ID NO: 45)
ACAAACCTTTTCGAGGCAAAAGGCAAAAAAGGCTGCTCTGGGATTCTCTTCAGCCAATCT
TCAATGCTCAAGTGTCTGAAGCAGCCATGGCAGAAGTACCTAAGCTCGCCAGTGAAATGA
TGGCTTATTACAGTGGCAATGAGGATGACTTGTTCTTTGAAGCTGATGGCCCTAAACAGA
TGAAGTGCTCCTTCCAGGACCTGGACCTCTGCCCTCTGGATGGCGGCATCCAGCTACGAA
TCTCCGACCACCACTACAGCAAGGGCTTCAGGCAGGCCGCGTCAGTTGTTGTGGCCATGG
ACAAGCTGAGGAAGATGCTGGTTCCCTGCCCACAGACCTTCCAGGAGAATGACCTGAGCA
CCTTCTTTCCCTTCATCTTTGAAGAAGAACCTATCTTCTTCGACACATGGGATAACGAGG
CTTATGTGCACGATGCACCTGTACGATCACTGAACTGCACGCTCCGGGACTCACAGCAAA
AAAGCTTGGTGATGTCTGGTCCATATGAACTGAAAGCTCTCCACCTCCAGGGACAGGATA
TGGAGCAACAAGTGGTGTTCTCCATGTCCTTTGTACAAGGAGAAGAAAGTAATGACAAAA
TACCTGTGGCCTTGGGCCTCAAGGAAAAGAATCTGTACCTGTCCTGCGTGTTGAAAGATG
ATAAGCCCACTCTACAGCTGGAGAGTGTAGATCCCAAAAATTACCCAAAGAAGAAGATGG
AAAAGCGATTTGTCTTCAACAAGATAGAAATCAATAACAAGCTGGAATTTGAGTCTGCCC
AGTTCCCCAACTGGTACATCAGCACCTCTCAAGCAGAAAACATGCCCGTCTTCCTGGGAG
GGACCAAAGGCGGCCAGGATATAACTGACTTCACCATGCAATTTGTGTCTTCCTAAAGAG
AGCTGTACCCAGAGAGTCCTGTGCTGAATGTGGACTCAATCCCTAGGGCTGGCAGAAAGG
GAACAGAAAGGTTTTTGAGTACGGCTATAGCCTGGACTTTCCTGTTGTCTACACCAATGC
CCAACTGCCTGCCTTAGGGTAGTGCTAAGAGGATCTCCTGTCCATCAGCCAGGACAGTCA
GCTCTCTCCTTTCAGGGCCAATCCCAGCCCTTTTGTTGAGCCAGGCCTCTCTCACCTCTC
CTACTCACTTAAAGCCCGCCTGACAGAAACCAGGCCACATTTTGGTTCTAAGAAACCCTC
CTCTGTCATTCGCTCCCACATTCTGATGAGCAACCGCTTCCCTATTTATTTATTTATTTG
TTTGTTTGTTTTGATTCATTGGTCTAATTTATTCAAAGGGGGCAAGAAGTAGCAGTGTCT
GTAAAAGAGCCTAGTTTTTAATAGCTATGGAATCAATTCAATTTGGACTGGTGTGCTCTC
TTTAAATCAAGTCCTTTAATTAAGACTGAAAATATATAAGCTCAGATTATTTAAATGGGA
ATATTTATAAATGAGCAAATATCATACTGTTCAATGGTTCTCAAATAAACTTCACT

As used herein, the term “IL1RAP” refers to the gene encoding Interleukin-1 receptor accessory protein. The terms “IL1 RAP” and “Interleukin-1 receptor accessory protein” include wild-type forms of the IL1 RAP gene, as well as variants (e.g., splice variants and polymorphisms) of wild-type IL1 RAP. Examples of such variants are nucleic acids having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to a wild-type IL1RAP nucleic acid sequence (e.g., SEQ ID NO: 46, ENA accession number AF029213). SEQ ID NO: 46 is a wild-type gene sequence encoding IL1 RAP protein, and is shown below:

(SEQ ID NO: 46)
TCTCAAAGGATGACACTTCTGTGGTGTGTAGTGAGTCTCTACTTTTATGGAATCCTGCAA
AGTGATGCCTCAGAACGCTGCGATGACTGGGGACTAGACACCATGAGGCAAATCCAAGTG
TTTGAAGATGAGCCAGCTCGCATCAAGTGCCCACTCTTTGAACACTTCTTGAAATTCAAC
TACAGCACAGCCCATTCAGCTGGCCTTACTCTGATCTGGTATTGGACTAGGCAGGACCGG
GACCTTGAGGAGCCAATTAACTTCCGCCTCCCCGAGAACCGCATTAGTAAGGAGAAAGAT
GTGCTGTGGTTCCGGCCCACTCTCCTCAATGACACTGGCAACTATACCTGCATGTTAAGG
AACACTACATATTGCAGCAAAGTTGCATTTCCCTTGGAAGTTGTTCAAAAAGACAGCTGT
TTCAATTCCCCCATGAAACTCCCAGTGCATAAACTGTATATAGAATATGGCATTCAGAGG
ATCACTTGTCCAAATGTAGATGGATATTTTCCTTCCAGTGTCAAACCGACTATCACTTGG
TATATGGGCTGTTATAAAATACAGAATTTTAATAATGTAATACCCGAAGGTATGAACTTG
AGTTTCCTCATTGCCTTAATTTCAAATAATGGAAATTACACATGTGTTGTTACATATCCA
GAAAATGGACGTACGTTTCATCTCACCAGGACTCTGACTGTAAAGGTAGTAGGCTCTCCA
AAAAATGCAGTGCCCCCTGTGATCCATTCACCTAATGATCATGTGGTCTATGAGAAAGAA
CCAGGAGAGGAGCTACTCATTCCCTGTACGGTCTATTTTAGTTTTCTGATGGATTCTCGC
AATGAGGTTTGGTGGACCATTGATGGAAAAAAACCTGATGACATCACTATTGATGTCACC
ATTAACGAAAGTATAAGTCATAGTAGAACAGAAGATGAAACAAGAACTCAGATTTTGAGC
ATCAAGAAAGTTACCTCTGAGGATCTCAAGCGCAGCTATGTCTGTCATGCTAGAAGTGCC
AAAGGCGAAGTTGCCAAAGCAGCCAAGGTGAAGCAGAAAGTGCCAGCTCCAAGATACACA
GTGGAACTGGCTTGTGGTTTTGGAGCCACAGTCCTGCTAGTGGTGATTCTCATTGTTGTT
TACCATGTTTACTGGCTAGAGATGGTCCTATTTTACCGGGCTCATTTTGGAACAGATGAA
ACCATTTTAGATGGAAAAGAGTATGATATTTATGTATCCTATGCAAGGAATGCGGAAGAA
GAAGAATTTGTATTACTGACCCTCCGTGGAGTTTTGGAGAATGAATTTGGATACAAGCTG
TGCATCTTTGACCGAGACAGTCTGCCTGGGGGAATTGTCACAGATGAGACTTTGAGCTTC
ATTCAGAAAAGCAGACGCCTCCTGGTTGTTCTAAGCCCCAACTACGTGCTCCAGGGAACC
CAAGCCCTCCTGGAGCTCAAGGCTGGCCTAGAAAATATGGCCTCTCGGGGCAACATCAAC
GTCATTTTAGTACAGTACAAAGCTGTGAAGGAAACGAAGGTGAAAGAGCTGAAGAGGGCT
AAGACGGTGCTCACGGTCATTAAATGGAAAGGGGAAAAATCCAAGTATCCACAGGGCAGG
TTCTGGAAGCAGCTGCAGGTGGCCATGCCAGTGAAGAAAAGTCCCAGGCGGTCTAGCAGT
GATGAGCAGGGCCTCTCGTATTCATCTTTGAAAAATGTATGAAAGGAATAATGAAAAGGA

As used herein, the term “INPP5D” refers to the gene encoding Phosphatidylinositol 3,4,5-trisphosphate 5-phosphatase 1. The terms “INPP5D” and “Phosphatidylinositol 3,4,5-trisphosphate 5-phosphatase 1” include wild-type forms of the INPP5D gene, as well as variants (e.g., splice variants and polymorphisms) of wild-type INPP5D. Examples of such variants are nucleic acids having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to a wild-type INPP5D nucleic acid sequence (e.g., SEQ ID NO: 47, ENA accession number X98429). SEQ ID NO: 47 is a wild-type gene sequence encoding INPP5D protein, and is shown below:

(SEQ ID NO: 47)
GTTGCTGTCGCCGTTGCTGTCGGCCGAGGCCACCAAGAGGCAACGGGGGGCAGGTTGCAG
TGGAGGGGCCTCCGCTCCCCTCGGTGGTGTGTGGGTCCTGGGGGTGCCTGCCGGCCCAGC
CGAGGAGGCCCACGCCCACCATGGTCCCCTGCTGGAACCATGGCAACATCACCCGCTCCA
AGGCGGAGGAGCTGCTTTCCAGGACAGGCAAGGACGGGAGCTTCCTCGTGCGTGCCAGCG
AGTCCATCTCCCGGGCATACGCGCTCTGCGTGCTGTATCGGAATTGCGTTTACACTTACA
GAATTCTGCCCAATGAAGATGATAAATTCACTGTTCAGGCATCCGAAGGCGTCTCCATGA
GGTTCTTCACCAAGCTGGACCAGCTCATCGAGTTTTACAAGAAGGAAAACATGGGGCTGG
TGACCCATCTGCAATACCCTGTGCCGCTGGAGGAAGAGGACACAGGCGACGACCCTGAGG
AGGACACAGAAAGTGTCGTGTCTCCACCCGAGCTGCCCCCAAGAAACATCCCGCTGACTG
CCAGCTCCTGTGAGGCCAAGGAGGTTCCTTTTTCAAACGAGAATCCCCGAGCGACCGAGA
CCAGCCGGCCGAGCCTCTCCGAGACATTGTTCCAGCGACTGCAAAGCATGGACACCAGTG
GGCTTCCAGAAGAGCATCTTAAGGCCATCCAAGATTATTTAAGCACTCAGCTCGCCCAGG
ACTCTGAATTTGTGAAGACAGGGTCCAGCAGTCTTCCTCACCTGAAGAAACTGACCACAC
TGCTCTGCAAGGAGCTCTATGGAGAAGTCATCCGGACCCTCCCATCCCTGGAGTCTCTGC
AGAGGTTATTTGACCAGCAGCTCTCCCCGGGCCTCCGTCCACGTCCTCAGGTTCCTGGTG
AGGCCAATCCCATCAACATGGTGTCCAAGCTCAGCCAACTGACAAGCCTGTTGTCGTCCA
TTGAAGACAAGGTCAAGGCCTTGCTGCACGAGGGTCCTGAGTCTCCGCACCGGCCCTCCC
TTATCCCTCCAGTCACCTTTGAGGTGAAGGCAGAGTCTCTGGGGATTCCTCAGAAAATGC
AGCTCAAAGTCGACGTTGAGTCTGGGAAACTGATCATTAAGAAGTCCAAGGATGGTTCTG
AGGACAAGTTCTACAGCCACAAGAAAATCCTGCAGCTGATTAAGTCACAGAAATTTCTGA
ATAAGTTGGTGATCTTGGTGGAAACGGAGAAGGAGAAGATCCTGCGGAAGGAATATGTTT
TTGCTGACTCCAAAAAGAGAGAAGGCTTCTGCCAGCTCCTGCAGCAGATGAAGAACAAGC
ACTCAGAGCAGCCGGAGCCCGACATGATCACCATCTTCATCGGCACCTGGAACATGGGTA
ACGCCCCCCCTCCCAAGAAGATCACGTCCTGGTTTCTCTCCAAGGGGCAGGGAAAGACGC
GGGACGACTCTGCGGACTACATCCCCCATGACATTTACGTGATCGGCACCCAAGAGGACC
CCCTGAGTGAGAAGGAGTGGCTGGAGATCCTCAAACACTCCCTGCAAGAAATCACCAGTG
TGACTTTTAAAACAGTCGCCATCCACACGCTCTGGAACATCCGCATCGTGGTGCTGGCCA
AGCCTGAGCACGAGAACCGGATCAGCCACATCTGTACTGACAACGTGAAGACAGGCATTG
CAAACACACTGGGGAACAAGGGAGCCGTGGGGGTGTCGTTCATGTTCAATGGAACCTCCT
TAGGGTTCGTCAACAGCCACTTGACTTCAGGAAGTGAAAAGAAACTCAGGCGAAACCAAA
ACTATATGAACATTCTCCGGTTCCTGGCCCTGGGCGACAAGAAGCTGAGTCCCTTTAACA
TCACTCACCGCTTCACGCACCTCTTCTGGTTTGGGGATOTTAACTACCGTGTGGATCTGC
CTACCTGGGAGGCAGAAACCATCATCCAGAAAATCAAGCAGCAGCAGTACGCAGACCTCC
TGTCCCACGACCAGCTGCTCACAGAGAGGAGGGAGCAGAAGGTCTTCCTACACTTCGAGG
AGGAAGAAATCACGTTTGCCCCAACCTACCGTTTTGAGAGACTGACTCGGGACAAATACG
CCTACACCAAGCAGAAAGCGACAGGGATGAAGTACAACTTGCCTTCCTGGTGTGACCGAG
TCCTCTGGAAGTCTTATCCCCTGGTGCACGTGGTGTGTCAGTCTTATGGCAGTACCAGCG
ACATCATGACGAGTGACCACAGCCCTGTCTTTGCCACATTTGAGGCAGGAGTCACTTCCC
AGTTTGTCTCCAAGAACGGTCCCGGGACTGTTGACAGCCAAGGACAGATTGAGTTTCTCA
GGTGCTATGCCACATTGAAGACCAAGTCCCAGACCAAATTCTACCTGGAGTTCCACTCGA
GCTGCTTGGAGAGTTTTGTCAAGAGTCAGGAAGGAGAAAATGAAGAAGGAAGTGAGGGGG
AGCTGGTGGTGAAGTTTGGTGAGACTCTTCCAAAGCTGAAGCCCATTATCTCTGACCCTG
AGTACCTGCTAGACCAGCACATCCTCATCAGCATCAAGTCCTCTGACAGCGACGAATCCT
ATGGCGAGGGCTGCATTGCCCTTCGGTTAGAGGCCACAGAAACGCAGCTGCCCATCTACA
CGCCTCTCACCCACCATGGGGAGTTGACAGGCCACTTCCAGGGGGAGATCAAGCTGCAGA
CCTCTCAGGGCAAGACGAGGGAGAAGCTCTATGACTTTGTGAAGACGGAGCGTGATGAAT
CCAGTGGGCCAAAGACCCTGAAGAGCCTCACCAGCCACGACCCCATGAAGCAGTGGGAAG
TCACTAGCAGGGCCCCTCCGTGCAGTGGCTCCAGCATCACTGAAATCATCAACCCCAACT
ACATGGGAGTGGGGCCCTTTGGGCCACCAATGCCCCTGCACGTGAAGCAGACCTTGTCCC
CTGACCAGCAGCCCACAGCCTGGAGCTACGACCAGCCGCCCAAGGACTCCCCGCTGGGGC
CCTGCAGGGGAGAAAGTCCTCCGACACCTCCCGGCCAGCCGCCCATATCACCCAAGAAGT
TTTTACCCTCAACAGCAAACCGGGGTCTCCCTCCCAGGACACAGGAGTCAAGGCCCAGTG
ACCTGGGGAAGAACGCAGGGGACACGCTGCCTCAGGAGGACCTGCCGCTGACGAAGCCCG
AGATGTTTGAGAACCCCCTGTATGGGTCCCTGAGTTCCTTCCCTAAGCCTGCTCCCAGGA
AGGACCAGGAATCCCCCAAAATGCCGCGGAAGGAACCCCCGCCCTGCCCGGAACCCGGCA
TCTTGTCGCCCAGCATCGTGCTCACCAAAGCCCAGGAGGCTGATCGCGGCGAGGGGCCCG
GCAAGCAGGTGCCCGCGCCCCGGCTGCGCTCCTTCACGTGCTCATCCTCTGCCGAGGGCA
GGGCGGCCGGGGGGACAAGAGCCAAGGGAAGCCCAAGACCCCGGTCAGCTCCCAGGCCC
CGGTGCCGGCCAAGAGGCCCATCAAGCCTTCCAGATCGGAAATCAACCAGCAGACCCCGC
CCACCCCGACGCCGCGGCCGCCGCTGCCAGTCAAGAGCCCGGCGGTGCTGCACCTCCAGC
ACTCCAAGGGCCGCGACTACCGCGACAACACCGAGCTCCCGTATCACGGCAAGCACCGGC
CGGAGGAGGGGCCACCAGGGCCTCTAGGCAGGACTGCCATGCAGTGAAGCCCTCAGTGAG
CTGCCACTGAGTCGGGAGCCCAGAGGAACGGCGTGAAGCCACTGGACCCTCTCCCGGGAC
CTCCTGCTGGCTCCTCCTGCCCAGCTTCCTATGCAAGGCTTTGTGTTTTCAGGAAAGGGC
CTAGCTTCTGTGTGGCCCACAGAGTTCACTGCCTGTGAGACTTAGCACCAAGTGCTGAGG
CTGGAAGAAAAACGCACACCAGACGGGCAACAAACAGTCTGGGTCCCCAGCTCGCTCTTG
GTACTTGGGACCCCAGTGCCTTGTTGAGGGCGCCATTCTGAAGAAAGGAACTGCAGCGCC
GATTTGAGGGTGGAGATATAGATAATAATAATATTAATAATAATAATGGCCACATGGATC
GAACACTCATGGTGTGCCAAGTGCTGTGCTAAGTGCTTTACGAACATTCGTCATATCAGG
ATGACCTCGAGAGCTGAGGCTCTAGCACCTAAAACCACGTGCCCAAACCCACCAGTTTAA
AACGGTGTGTGTTCGGAGGGGTGAAAGCATTAAGAAGCCCAGTGCCCTCCTGGAGTGAGA
CAAGGGCTCGGCCTTAAGGAGCTGAAGAGTCTGGGTAGCTTGTTTAGGGTACAAGAAGCC
TGTTCTGTCCAGCTTCAGTGACACAAGCTGCTTTAGCTAAAGTCCCGCGGGTTCCGGCAT
GGCTAGGCTGAGAGCAGGGATCTACCTGGCTTCTCAGTTCTTTGGTTGGAAGGAGCAGGA
AATCAGCTCCTATTCTCCAGTGGAGAGATCTGGCCTCAGCTTGGGCTAGAGATGCCAAGG
CCTGTGCCAGGTTCCCTGTGCCCTCCTCGAGGTGGGCAGCCATCACCAGCCACAGTTAAG
CCAAGCCCCCCAACATGTATTCCATCGTGCTGGTAGAAGAGTCTTTGCTGTTGCTCCCGA
AAGCCGTGCTCTCCAGCCTGGCTGCCAGGGAGGGTGGGCCTCTTGGTTCCAGGCTCTTGA
AATAGTGCAGCCTTTTCTTCCTATCTCTGTGGCTTTCAGCTCTGCTTCCTTGGTTATTAG
GAGAATAGATGGGTGATGTCTTTCCTTATGTTGCTTTTTCAACATAGCAGAATTAATGTA
GGGAGCTAAATCCAGTGGTGTGTGTGAATGCAGAAGGGAATGCACCCCACATTCCCATGA
TGGAAGTCTGCGTAACCAATAAATTGTGCCTTTCTCACTCAAAACC

As used herein, the term “ITGAM” refers to the gene encoding Integrin Subunit Alpha M. The terms “ITGAM” and “Integrin Subunit Alpha M” include wild-type forms of the ITGAM gene, as well as variants (e.g., splice variants and polymorphisms) of wild-type ITGAM. Examples of such variants are nucleic acids having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to a wild-type ITGAM nucleic acid sequence (e.g., SEQ ID NO: 48, NCBI Reference Sequence: NM_000632.3). SEQ ID NO: 48 is a wild-type gene sequence encoding ITGAM protein, and is shown below:

(SEQ ID NO: 48)
TTTTCTGCCCTTCTTTGCTTTGGTGGCTTCCTTGTGGTTCCTCAGTGGTGCCTGCAACCCCTGGTTCA
CCTCCTTCCAGGTTCTGGCTCCTTCCAGCCATGGCTCTCAGAGTCCTTCTGTTAACAGCCTTGACCT
TATGTCATGGGTTCAACTTGGACACTGAAAACGCAATGACCTTCCAAGAGAACGCAAGGGGCTTCGG
GCAGAGCGTGGTCCAGCTTCAGGGATCCAGGGTGGTGGTTGGAGCCCCCCAGGAGATAGTGGCTG
CCAACCAAAGGGGCAGCCTCTACCAGTGCGACTACAGCACAGGCTCATGCGAGCCCATCCGCCTGC
AGGTCCCCGTGGAGGCCGTGAACATGTCCCTGGGCCTGTCCCTGGCAGCCACCACCAGCCCCCCT
CAGCTGCTGGCCTGTGGTCCCACCGTGCACCAGACTTGCAGTGAGAACACGTATGTGAAAGGGCTC
TGCTTCCTGTTTGGATCCAACCTACGGCAGCAGCCCCAGAAGTTCCCAGAGGCCCTCCGAGGGTGT
CCTCAAGAGGATAGTGACATTGCCTTCTTGATTGATGGCTCTGGTAGCATCATCCCACATGACTTTCG
GCGGATGAAGGAGTTTGTCTCAACTGTGATGGAGCAATTAAAAAAGTCCAAAACCTTGTTCTCTTTGA
TGCAGTACTCTGAAGAATTCCGGATTCACTTTACCTTCAAAGAGTTCCAGAACAACCCTAACCCAAGA
TCACTGGTGAAGCCAATAACGCAGCTGCTTGGGCGGACACACACGGCCACGGGCATCCGCAAAGT
GGTACGAGAGCTGTTTAACATCACCAACGGAGCCCGAAAGAATGCCTTTAAGATCCTAGTTGTCATC
ACGGATGGAGAAAAGTTTGGCGATCCCTTGGGATATGAGGATGTCATCCCTGAGGCAGACAGAGAG
GGAGTCATTCGCTACGTCATTGGGGTGGGAGATGCCTTCCGCAGTGAGAAATCCCGCCAAGAGCTT
AATACCATCGCATCCAAGCCGCCTCGTGATCACGTGTTCCAGGTGAATAACTTTGAGGCTCTGAAGA
CCATTCAGAACCAGCTTCGGGAGAAGATCTTTGCGATCGAGGGTACTCAGACAGGAAGTAGCAGCT
CCTTTGAGCATGAGATGTCTCAGGAAGGCTTCAGCGCTGCCATCACCTCTAATGGCCCCTTGCTGAG
CACTGTGGGGAGCTATGACTGGGCTGGTGGAGTCTTTCTATATACATCAAAGGAGAAAAGCACCTTC
ATCAACATGACCAGAGTGGATTCAGACATGAATGATGCTTACTTGGGTTATGCTGCCGCCATCATCTT
ACGGAACCGGGTGCAAAGCCTGGTTCTGGGGGCACCTCGATATCAGCACATCGGCCTGGTAGCGAT
GTTCAGGCAGAACACTGGCATGTGGGAGTCCAACGCTAATGTCAAGGGCACCCAGATCGGCGCCTA
CTTCGGGGCCTCCCTCTGCTCCGTGGACGTGGACAGCAACGGCAGCACCGACCTGGTCCTCATCG
GGGCCCCCCATTACTACGAGCAGACCCGAGGGGGCCAGGTGTCCGTGTGCCCCTTGCCCAGGGGG
AGGGCTCGGTGGCAGTGTGATGCTGTTCTCTACGGGGAGCAGGGCCAACCCTGGGGCCGCTTTGG
GGCAGCCCTAACAGTGCTGGGGGACGTAAATGGGGACAAGCTGACGGACGTGGCCATTGGGGCCC
CAGGAGAGGAGGACAACCGGGGTGCTGTTTACCTGTTTCACGGAACCTCAGGATCTGGCATCAGCC
CCTCCCATAGCCAGCGGATAGCAGGCTCCAAGCTCTCTCCCAGGCTCCAGTATTTTGGTCAGTCACT
GAGTGGGGGCCAGGACCTCACAATGGATGGACTGGTAGACCTGACTGTAGGAGCCCAGGGGCACG
TGCTGCTGCTCAGGTCCCAGCCAGTACTGAGAGTCAAGGCAATCATGGAGTTCAATCCCAGGGAAG
TGGCAAGGAATGTATTTGAGTGTAATGATCAGGTGGTGAAAGGCAAGGAAGCCGGAGAGGTCAGAG
TCTGCCTCCATGTCCAGAAGAGCACACGGGATCGGCTAAGAGAAGGACAGATCCAGAGTGTTGTGA
CTTATGACCTGGCTCTGGACTCCGGCCGCCCACATTCCCGCGCCGTCTTCAATGAGACAAAGAACA
GCACACGCAGACAGACACAGGTCTTGGGGCTGACCCAGACTTGTGAGACCCTGAAACTACAGTTGC
CGAATTGCATCGAGGACCCAGTGAGCCCCATTGTGCTGCGCCTGAACTTCTCTCTGGTGGGAACGC
CATTGTCTGCTTTCGGGAACCTCCGGCCAGTGCTGGCGGAGGATGCTCAGAGACTCTTCACAGCCT
TGTTTCCCTTTGAGAAGAATTGTGGCAATGACAACATCTGCCAGGATGACCTCAGCATCACCTTCAGT
TTCATGAGCCTGGACTGCCTCGTGGTGGGTGGGCCCCGGGAGTTCAACGTGACAGTGACTGTGAGA
AATGATGGTGAGGACTCCTACAGGACACAGGTCACCTTCTTCTTCCCGCTTGACCTGTCCTACCGGA
AGGTGTCCACGCTCCAGAACCAGCGCTCACAGCGATCCTGGCGCCTGGCCTGTGAGTCTGCCTCCT
CCACCGAAGTGTCTGGGGCCTTGAAGAGCACCAGCTGCAGCATAAACCACCCCATCTTCCCGGAAA
ACTCAGAGGTCACCTTTAATATCACGTTTGATGTAGACTCTAAGGCTTCCCTTGGAAACAAACTGCTC
CTCAAGGCCAATGTGACCAGTGAGAACAACATGCCCAGAACCAACAAAACCGAATTCCAACTGGAGC
TGCCGGTGAAATATGCTGTCTACATGGTGGTCACCAGCCATGGGGTCTCCACTAAATATCTCAACTT
CACGGCCTCAGAGAATACCAGTCGGGTCATGCAGCATCAATATCAGGTCAGCAACCTGGGGCAGAG
GAGCCTCCCCATCAGCCTGGTGTTCTTGGTGCCCGTCCGGCTGAACCAGACTGTCATATGGGACCG
CCCCCAGGTCACCTTCTCCGAGAACCTCTCGAGTACGTGCCACACCAAGGAGCGCTTGCCCTCTCA
CTCCGACTTTCTGGCTGAGCTTCGGAAGGCCCCCGTGGTGAACTGCTCCATCGCTGTCTGCCAGAG
AATCCAGTGTGACATCCCGTTCTTTGGCATCCAGGAAGAATTCAATGCTACCCTCAAAGGCAACCTC
TCGTTTGACTGGTACATCAAGACCTCGCATAACCACCTCCTGATCGTGAGCACAGCTGAGATCTTGT
TTAACGATTCCGTGTTCACCCTGCTGCCGGGACAGGGGGCGTTTGTGAGGTCCCAGACGGAGACCA
AAGTGGAGCCGTTCGAGGTCCCCAACCCCCTGCCGCTCATCGTGGGCAGCTCTGTCGGGGGACTG
CTGCTCCTGGCCCTCATCACCGCCGCGCTGTACAAGCTCGGCTTCTTCAAGCGGCAATACAAGGAC
ATGATGAGTGAAGGGGGTCCCCCGGGGGCCGAACCCCAGTAGCGGCTCCTTCCCGACAGAGCTGC
CTCTCGGTGGCCAGCAGGACTCTGCCCAGACCACACGTAGCCCCCAGGCTGCTGGACACGTCGGA
CAGCGAAGTATCCCCGACAGGACGGGCTTGGGCTTCCATTTGTGTGTGTGCAAGTGTGTATGTGCG
TGTGTGCAAGTGTCTGTGTGCAAGTGTGTGCACATGTGTGCGTGTGCGTGCATGTGCACTTGCACG
CCCATGTGTGAGTGTGTGCAAGTATGTGAGTGTGTCCAAGTGTGTGTGCGTGTGTCCATGTGTGTGC
AAGTGTGTGCATGTGTGCGAGTGTGTGCATGTGTGTGCTCAGGGGCGTGTGGCTCACGTGTGTGAC
TCAGATGTCTCTGGCGTGTGGGTAGGTGACGGCAGCGTAGCCTCTCCGGCAGAAGGGAACTGCCT
GGGCTCCCTTGTGCGTGGGTGAAGCCGCTGCTGGGTTTTCCTCCGGGAGAGGGGACGGTCAATCC
TGTGGGTGAAGACAGAGGGAAACACAGCAGCTTCTCTCCACTGAAAGAAGTGGGACTTCCCGTCGC
CTGCGAGCCTGCGGCCTGCTGGAGCCTGCGCAGCTTGGATGGAGACTCCATGAGAAGCCGTGGGT
GGAACCAGGAACCTCCTCCACACCAGCGCTGATGCCCAATAAAGATGCCCACTGAGGAATGATGAA
GCTTCCTTTCTGGATTCATTTATTATTTCAATGTGACTTTAATTTTTTGGATGGATAAGCTTGTCTATGG
TACAAAAATCACAAGGCATTCAAGTGTACAGTGAAAAGTCTCCCTTTCCAGATATTCAAGTCACCTCC
TTAAAGGTAGTCAAGATTGTGTTTTGAGGTTTCCTTCAGACAGATTCCAGGCGATGTGCAAGTGTATG
CACGTGTGCACACACACCACACATACACACACACAAGCTTTTTTACACAAATGGTAGCATACTTTATA
TTGGTCTGTATCTTGCTTTTTTTCACCAATATTTCTCAGACATCGGTTCATATTAAGACATAAATTACTT
TTTCATTCTTTTATACCGCTGCATAGTATTCCATTGTGTGAGTGTACCATAATGTATTTAACCAGTCTT
CTTTTGATATACTATTTTCATTCTCTTGTTATTGCATCAATGCTGAGTTAATAAATCAAATATATGTCAT
TTTTGCATATATGTAAGGATAA

As used herein, the term “ITGAX” refers to the gene encoding Integrin alpha-X. The terms “ITGAX” and “Integrin alpha-X” include wild-type forms of the ITGAX gene, as well as variants (e.g., splice variants and polymorphisms) of wild-type ITGAX. Examples of such variants are nucleic acids having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to a wild-type ITGAX nucleic acid sequence (e.g., SEQ ID NO: 49, ENA accession number M81695). SEQ ID NO: 49 is a wild-type gene sequence encoding ITGAX protein, and is shown below:

(SEQ ID NO: 49)
GAATTCCTGCCACTCTTCCTGCAACGGCCCAGGAGCTCAGAGCTCCACATCTGACCTTCT
AGTCATGACCAGGACCAGGGCAGCACTCCTCCTGTTCACAGCCTTAGCAACTTCTCTAGG
TTTCAACTTGGACACAGAGGAGCTGACAGCCTTCCGTGTGGACAGCGCTGGGTTTGGAGA
CAGCGTGGTCCAGTATGCCAACTCCTGGGTGGTGGTTGGAGCCCCCCAAAAGATAACAGC
TGCCAACCAAACGGGTGGCCTCTACCAGTGTGGCTACAGCACTGGTGCCTGTGAGCCCAT
CGGCCTGCAGGTGCCCCCGGAGGCCGTGAACATGTCCCTGGGCCTGTCCCTGGCGTCTAC
CACCAGCCCTTCCCAGCTGCTGGCCTGCGGCCCCACCGTGCACCACGAGTGCGGGAGGAA
CATGTACCTCACCGGACTCTGCTTCCTCCTGGGCCCCACCCAGCTCACCCAGAGGCTCCC
GGTGTCCAGGCAGGAGTGCCCAAGACAGGAGCAGGACATTGTGTTCCTGATCGATGGCTC
AGGCAGCATCTCCTCCCGCAACTTTGCCACGATGATGAACTTCGTGAGAGCTGTGATAAG
CCAGTTCCAGAGACCCAGCACCCAGTTTTCCCTGATGCAGTTCTCCAACAAATTCCAAAC
ACACTTCACTTTCGAGGAATTCAGGCGCACGTCAAACCCCCTCAGCCTGTTGGCTTCTGT
TCACCAGCTGCAAGGGTTTACATACACGGCCACCGCCATCCAAAATGTCGTGCACCGATT
GTTCCATGCCTCATATGGGGCCCGTAGGGATGCCACCAAAATTCTCATTGTCATCACTGA
TGGGAAGAAAGAAGGCGACAGCCTGGATTATAAGGATGTCATCCCCATGGCTGATGCAGC
AGGCATCATCCGCTATGCAATTGGGGTTGGATTAGCTTTTCAAAACAGAAATTCTTGGAA
AGAATTAAATGACATTGCATCGAAGCCCTCCCAGGAACACATATTTAAAGTGGAGGACTT
TGATGCTCTGAAAGATATTCAAAACCAACTGAAGGAGAAGATCTTTGCCATTGAGGGTAC
GGAGACCACAAGCAGTAGCTCCTTCGAATTGGAGATGGCACAGGAGGGCTTCAGCGCTGT
GTTCACACCTGATGGCCCCGTTCTGGGGGCTGTGGGGAGCTTCACCTGGTCTGGAGGTGC
CTTCCTGTACCCCCCAAATATGAGCCCTACCTTCATCAACATGTCTCAGGAGAATGTGGA
CATGAGGGACTCTTACCTGGGTTACTCCACCGAGCTGGCCCTCTGGAAAGGGGTGCAGAG
CCTGGTCCTGGGGGCCCCCCGCTACCAGCACACCGGGAAGGCTGTCATCTTCACCCAGGT
GTCCAGGCAATGGAGGATGAAGGCCGAAGTCACGGGGACTCAGATCGGCTCCTACTTCGG
GGCCTCCCTCTGCTCCGTGGACGTAGACACCGACGGCAGCACCGACCTGGTCCTCATCGG
GGCCCCCCATTACTACGAGCAGACCCGAGGGGGCCAGGTGTCTGTGTGTCCCTTGCCCAG
GGGGTGGAGAAGGTGGTGGTGTGATGCTGTTCTCTACGGGGAGCAGGGCCACCCCTGGGG
TCGCTTTGGGGCGGCTCTGACAGTGCTGGGGGATGTGAATGGGGACAAGCTGACAGACGT
GGTCATCGGGGCCCCAGGAGAGGAGGAGAACCGGGGTGCTGTCTACCTGTTTCACGGAGT
CTTGGGACCCAGCATCAGCCCCTCCCACAGCCAGCGGATCGCGGGCTCCCAGCTCTCCTC
CAGGCTGCAGTATTTTGGGCAGGCACTGAGCGGGGGTCAAGACCTCACCCAGGATGGACT
GGTGGACCTGGCTGTGGGGGCCCGGGGCCAGGTGCTCCTGCTCAGGACCAGACCTGTGCT
CTGGGTGGGGGTGAGCATGCAGTTCATACCTGCCGAGATCCCCAGGTCTGCGTTTGAGTG
TCGGGAGCAGGTGGTCTCTGAGCAGACCCTGGTACAGTCCAACATCTGCCTTTACATTGA
CAAACGTTCTAAGAACCTGCTTGGGAGCCGTGACCTCCAAAGCTCTGTGACCTTGGACCT
GGCCCTCGACCCTGGCCGCCTGAGTCCCCGTGCCACCTTCCAGGAAACAAAGAACCGGAG
TCTGAGCCGAGTCCGAGTCCTCGGGCTGAAGGCACACTGTGAAAACTTCAACCTGCTGCT
CCCGAGCTGCGTGGAGGACTCTGTGACCCCCATTACCTTGCGTCTGAACTTCACGCTGGT
GGGCAAGCCCCTCCTTGCCTTCAGAAACCTGCGGCCTATGCTGGCCGCACTGGCTCAGAG
ATACTTCACGGCCTCCCTACCCTTTGAGAAGAACTGTGGAGCCGACCATATCTGCCAGGA
CAATCTCGGCATCTCCTTCAGCTTCCCAGGCTTGAAGTCCCTGCTGGTGGGGAGTAACCT
GGAGCTGAACGCAGAAGTGATGGTGTGGAATGACGGGGAAGACTCCTACGGAACCACCAT
CACCTTCTCCCACCCCGCAGGACTGTCCTACCGCTACGTGGCAGAGGGCCAGAAACAAGG
GCAGCTGCGTTCCCTGCACCTGACATGTGACAGCGCCCCAGTTGGGAGCCAGGGCACCTG
GAGCACCAGCTGCAGAATCAACCACCTCATCTTCCGTGGCGGCGCCCAGATCACCTTCTT
GGCTACCTTTGACGTCTCCCCCAAGGCTGTCCTGGGAGACCGGCTGCTTCTGACAGCCAA
TGTGAGCAGTGAGAACAACACTCCCAGGACCAGCAAGACCACCTTCCAGCTGGAGCTCCC
GGTGAAGTATGCTGTCTACACTGTGGTTAGCAGCCACGAACAATTCACCAAATACCTCAA
CTTCTCAGAGTCTGAGGAGAAGGAAAGCCATGTGGCCATGCACAGATACCAGGTCAATAA
CCTGGGACAGAGGGACCTGCCTGTCAGCATCAACTTCTGGGTGCCTGTGGAGCTGAACCA
GGAGGCTGTGTGGATGGATGTGGAGGTCTCCCACCCCCAGAACCCATCCCTTCGGTGCTC
CTCAGAGAAAATCGCACCCCCAGCATCTGACTTCCTGGCGCACATTCAGAAGAATCCCGT
GCTGGACTGCTCCATTGCTGGCTGCCTGCGGTTCCGCTGTGACGTCCCCTCCTTCAGCGT
CCAGGAGGAGCTGGATTTCACCCTGAAGGGCAACCTCAGCTTTGGCTGGGTCCGCCAGAT
ATTGCAGAAGAAGGTGTCGGTCGTGAGTGTGGCTGAAATTACGTTCGACACATCCGTGTA
CTCCCAGCTTCCAGGACAGGAGGCATTTATGAGAGCTCAGACGACAACGGTGCTGGAGAA
GTACAAGGTCCACAACCCCACCCCCCTCATCGTAGGCAGCTCCATTGGGGGTCTGTTGCT
GCTGGCACTCATCACAGCGGTACTGTACAAAGTTGGCTTCTTCAAGCGTCAGTACAAGGA
AATGATGGAGGAGGCAAATGGACAAATTGCCCCAGAAAACGGGACACAGACCCCCAGCCC
GCCCAGTGAGAAATGATCCCTCTTTGCCTTGGACTTCTTCTCCCGCGATTTTCCCCACTT
ACTTACCCTCACCTGTCAGGCTGACGGGGAGGAACCACTGCACCACCGAGAGAGGCTGGG
ATGGGCCTGCTTCCTGTCTTTGGGAGAAAACGTCTTGCTTGGGAAGGGGCCTTTGTCTTG
TCAAGGTTCCAACTGGAAACCCTTAGGACAGGGTCCCTGCTGTGTTCCCCAAAAGGACTT
GACTTGCAATTTCTACCTAGAAATACATGGACAATACCCCCAGGCCTCAGTCTCCCTTCT
CCCATGAGGCACGAATGATCTTTCTTTCCTTTCCTTTTTTTTTTTTTTCTTTTCTTTTTT
TTTTTTTTTGAGACGGAGTCTCGCTCTGTCACCCAGGCTGGAGTGCAATGGCGTGATCTC
GGCTCGCTGCAACCTCCGCCTCCCGGGTTCAAGTAATTCTGCTGTCTCAGCCTCCTGCGT
AGCTGGGACTACAGGCACACGCCACCTCGCCCGGCCCGATCTTTCTAAAATACAGTTCTG
AATATGCTGCTCATCCCCACCTGTCTTCAACAGCTCCCCATTACCCTCAGGACAATGTCT
GAACTCTCCAGCTTCGCGTGAGAAGTCCCCTTCCATCCCAGAGGGTGGGCTTCAGGGCGC
ACAGCATGAGAGCCTCTGTGCCCCCATCACCCTCGTTTCCAGTGAATTAGTGTCATGTCA
GCATCAGCTCAGGGCTTCATCGTGGGGCTCTCAGTTCCGATTCCCCAGGCTGAATTGGGA
GTGAGATGCCTGCATGCTGGGTTCTGCACAGCTGGCCTCCCGCGGTTGGGTCAACATTGC
TGGCCTGGAAGGGAGGAGCGCCCTCTAGGGAGGGACATGGCCCCGGTGCGGCTGCAGCTC
ACCAGCCCCAGGGGCAGAAGAGACCCAACCACTTCCTATTTTTTGAGGCTATGAATATAG
TACCTGAAAAAATGCCAAGCACTAGATTATTTTTTTAAAAAGCGTACTTTAAATGTTTGT
GTTAATACACATTAAAACATCGCACAAAAACGATGCATCTACCGCTCCTTGGGAAATAAT
CTGAAAGGTCTAAAAATAAAAAAGCCTTCTGTGG

As used herein, the term “LILRB4” refers to the gene encoding Leukocyte immunoglobulin-like receptor subfamily B member 4. The terms “LILRB4” and “Leukocyte immunoglobulin-like receptor subfamily B member 4” include wild-type forms of the LILRB4 gene, as well as variants (e.g., splice variants and polymorphisms) of wild-type LILRB4. Examples of such variants are nucleic acids having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to a wild-type LILRB4 nucleic acid sequence (e.g., SEQ ID NO: 50, ENA accession number U91925). SEQ ID NO: 50 is a wild-type gene sequence encoding LILRB4 protein, and is shown below:

(SEQ ID NO: 50)
TGAGATGAGAGCTGCCGACAGTTGGGGGTCAAGGGAGGAGACGCCATGATCCCCACCTTC
ACGGCTCTGCTCTGCCTCGGGCTGAGTCTGGGCCCCAGGACCCACATGCAGGCAGGGCCC
CTCCCCAAACCCACCCTCTGGGCTGAGCCAGGCTCTGTGATCAGCTGGGGGAACTCTGTG
ACCATCTGGTGTCAGGGGACCCTGGAGGCTCGGGAGTACCGTCTGGATAAAGAGGAAAGC
CCAGCACCCTGGGACAGACAGAACCCACTGGAGCCCAAGAACAAGGCCAGATTCTCCATC
CCATCCATGACAGAGGACTATGCAGGGAGATACCGCTGTTACTATCGCAGCCCTGTAGGC
TGGTCACAGCCCAGTGACCCCCTGGAGCTGGTGATGACAGGAGCCTACAGTAAACCCACC
CTTTCAGCCCTGCCGAGTCCTCTTGTGACCTCAGGAAAGAGCGTGACCCTGCTGTGTCAG
TCACGGAGCCCAATGGACACTTTCCTTCTGATCAAGGAGCGGGCAGCCCATCCCCTACTG
CATCTGAGATCAGAGCACGGAGCTCAGCAGCACCAGGCTGAATTCCCCATGAGTCCTGTG
ACCTCAGTGCACGGGGGGACCTACAGGTGCTTCAGCTCACACGGCTTCTCCCACTACCTG
CTGTCACACCCCAGTGACCCCCTGGAGCTCATAGTCTCAGGATCCTTGGAGGGTCCCAGG
CCCTCACCCACAAGGTCCGTCTCAACAGCTGCAGGCCCTGAGGACCAGCCCCTCATGCCT
ACAGGGTCAGTCCCCCACAGTGGTCTGAGAAGGCACTGGGAGGTACTGATCGGGGTCTTG
GTGGTCTCCATCCTGCTTCTCTCCCTCCTCCTCTTCCTCCTCCTCCAACACTGGCGTCAG
GGAAAACACAGGACATTGGCCCAGAGACAGGCTGATTTCCAACGTCCTCCAGGGGCTGCC
GAGCCAGAGCCCAAGGACGGGGGCCTACAGAGGAGGTCCAGCCCAGCTGCTGACGTCCAG
GGAGAAAACTTCTGTGCTGCCGTGAAGAACACACAGCCTGAGGACGGGGTGGAAATGGAC
ACTCGGCAGAGCCCACACGATGAAGACCCCCAGGCAGTGACGTATGCCAAGGTGAAACAC
TCCAGACCTAGGAGAGAAATGGCCTCTCCTCCCTCCCCACTGTCTGGGGAATTCCTGGAC
ACAAAGGACAGACAGGCAGAAGAGGACAGACAGATGGACACTGAGGCTGCTGCATCTGAA
GCCCCCCAGGATGTGACCTACGCCCAGCTGCACAGCTTTACCCTCAGACAGAAGGCAACT
GAGCCTCCTCCATCCCAGGAAGGGGCCTCTCCAGCTGAGCCCAGTGTCTATGCCACTCTG
GCCATCCACTAATCCAGGGGGGACCCAGACCCCACAAGCCATGGAGACTCAGGACCCCAG
AAGGCATGGAAGCTGCCTCCAGTAGACATCACTGAACCCCAGCCAGCCCAGACCCCTGAC
ACAGACCACTAGAAGATTCCGGGAACGTTGGGAGTCACCTGATTCTGCAAAGATAAATAA
TATCCCTGCATTATCAAAATAAAGTAGCAGACCTCTCAATTCA

As used herein, the term “LPL” refers to the gene encoding Lipoprotein lipase. The terms “LPL” and “Lipoprotein lipase” include wild-type forms of the LPL gene, as well as variants (e.g., splice variants and polymorphisms) of wild-type LPL. Examples of such variants are nucleic acids having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to a wild-type LPL nucleic acid sequence (e.g., SEQ ID NO: 51, ENA accession number M15856). SEQ ID NO: 51 is a wild-type gene sequence encoding LPL protein, and is shown below:

(SEQ ID NO: 51)
CCCCTCTTCCTCCTCCTCAAGGGAAAGCTGCCCACTTCTAGCTGCCCTGCCATCCCCTTT
AAAGGGCGACTTGCTCAGCGCCAAACCGCGGCTCCAGCCCTCTCCAGCCTCCGGCTCAGC
CGGCTCATCAGTCGGTCCGCGCCTTGCAGCTCCTCCAGAGGGACGCGCCCCGAGATGGAG
AGCAAAGCCCTGCTCGTGCTGACTCTGGCCGTGTGGCTCCAGAGTCTGACCGCCTCCCGC
GGAGGGGTGGCCGCCGCCGACCAAAGAAGAGATTTTATCGACATCGAAAGTAAATTTGCC
CTAAGGACCCCTGAAGACACAGCTGAGGACACTTGCCACCTCATTCCCGGAGTAGCAGAG
TCCGTGGCTACCTGTCATTTCAATCACAGCAGCAAAACCTTCATGGTGATCCATGGCTGG
ACGGTAACAGGAATGTATGAGAGTTGGGTGCCAAAACTTGTGGCCGCCCTGTACAAGAGA
GAACCAGACTCCAATGTCATTGTGGTGGACTGGCTGTCACGGGCTCAGGAGCATTACCCA
GTGTCCGCGGGCTACACCAAACTGGTGGGACAGGATGTGGCCCGGTTTATCAACTGGATG
GAGGAGGAGTTTAACTACCCTCTGGACAATGTCCATCTCTTGGGATACAGCCTTGGAGCC
CATGCTGCTGGCATTGCAGGAAGTCTGACCAATAAGAAAGTCAACAGAATTACTGGCCTC
GATCCAGCTGGACCTAACTTTGAGTATGCAGAAGCCCCGAGTCGTCTTTCTCCTGATGAT
GCAGATTTTGTAGACGTCTTACACACATTCACCAGAGGGTCCCCTGGTCGAAGCATTGGA
ATCCAGAAACCAGTTGGGCATGTTGACATTTACCCGAATGGAGGTACTTTTCAGCCAGGA
TGTAACATTGGAGAAGCTATCCGCGTGATTGCAGAGAGAGGACTTGGAGATGTGGACCAG
CTAGTGAAGTGCTCCCACGAGCGCTCCATTCATCTCTTCATCGACTCTCTGTTGAATGAA
GAAAATCCAAGTAAGGCCTACAGGTGCAGTTCCAAGGAAGCCTTTGAGAAAGGGCTCTGC
TTGAGTTGTAGAAAGAACCGCTGCAACAATCTGGGCTATGAGATCAATAAAGTCAGAGCC
AAAAGAAGCAGCAAAATGTACCTGAAGACTCGTTCTCAGATGCCCTACAAAGTCTTCCAT
TACCAAGTAAAGATTCATTTTTCTGGGACTGAGAGTGAAACCCATACCAATCAGGCCTTT
GAGATTTCTCTGTATGGCACCGTGGCCGAGAGTGAGAACATCCCATTCACTCTGCCTGAA
GTTTCCACAAATAAGACCTACTCCTTCCTAATTTACACAGAGGTAGATATTGGAGAACTA
CTCATGTTGAAGCTCAAATGGAAGAGTGATTCATACTTTAGCTGGTCAGACTGGTGGAGC
AGTCCCGGCTTCGCCATTCAGAAGATCAGAGTAAAAGCAGGAGAGACTCAGAAAAAGGTG
ATCTTCTGTTCTAGGGAGAAAGTGTCTCATTTGCAGAAAGGAAAGGCACCTGCGGTATTT
GTGAAATGCCATGACAAGTCTCTGAATAAGAAGTCAGGCTGAAACTGGGCGAATCTACAG
AACAAAGAACGGCATGTGAATTCTGTGAAGAATGAAGTGGAGGAAGTAACTTTTACAAAA
CATACCCAGTGTTTGGGGTGTTTCAAAAGTGGATTTTCCTGAATATTAATCCCAGCCCTA
CCCTTGTTAGTTATTTTAGGAGACAGTCTCAAGCACTAAAAAGTGGCTAATTCAATTTAT
GGGGTATAGTGGCCAAATAGCACATCCTCCAACGTTAAAAGACAGTGGATCATGAAAAGT
GCTGTTTTGTCCTTTGAGAAAGAAATAATTGTTTGAGCGCAGAGTAAAATAAGGCTCCTT
CATGTGGCGTATTGGGCCATAGCCTATAATTGGTTAGAACCTCCTATTTTAATTGGAATT
CTGGATCTTTCGGACTGAGGCCTTCTCAAACTTTACTCTAAGTCTCCAAGAATACAGAAA
ATGCTTTTCCGCGGCACGAATCAGACTCATCTACACAGCAGTATGAATGATGTTTTAGAA
TGATTCCCTCTTGCTATTGGAATGTGGTCCAGACGTCAACCAGGAACATGTAACTTGGAG
AGGGACGAAGAAAGGGTCTGATAAACACAGAGGTTTTAAACAGTCCCTACCATTGGCCTG
CATCATGACAAAGTTACAAATTCAAGGAGATATAAAATCTAGATCAATTAATTCTTAATA
GGCTTTATCGTTTATTGCTTAATCCCTCTCTCCCCCTTCTTTTTTGTCTCAAGATTATAT
TATAATAATGTTCTCTGGGTAGGTGTTGAAAATGAGCCTGTAATCCTCAGCTGACACATA
ATTTGAATGGTGCAGAAAAAAAAAAGATACCGTAATTTTATTATTAGATTCTCCAAATGA
TTTTCATCAATTTAAAATCATTCAATATCTGACAGTTACTCTTCAGTTTTAGGCTTACCT
TGGTCATGCTTCAGTTGTACTTCCAGTGCGTCTCTTTTGTTCCTGGCTTTGACATGAAAA
GATAGGTTTGAGTTCAAATTTTGCATTGTGTGAGCTTCTACAGATTTTAGACAAGGACCG
TTTTTACTAAGTAAAAGGGTGGAGAGGTTCCTGGGGTGGATTCCTAAGCAGTGCTTGTAA
ACCATCGCGTGCAATGAGCCAGATGGAGTACCATGAGGGTTGTTATTTGTTGTTTTTAAC
AACTAATCAAGAGTGAGTGAACAACTATTTATAAACTAGATCTCCTATTTTTCAGAATGC
TCTTCTACGTATAAATATGAAATGATAAAGATGTCAAATATCTCAGAGGCTATAGCTGGG
AACCCGACTGTGAAAGTATGTGATATCTGAACACATACTAGAAAGCTCTGCATGTGTGTT
GTCCTTCAGCATAATTCGGAAGGGAAAACAGTCGATCAAGGGATGTATTGGAACATGTCG
GAGTAGAAATTGTTCCTGATGTGCCAGAACTTCGACCCTTTCTCTGAGAGAGATGATCGT
GCCTATAAATAGTAGGACCAATGTTGTGATTAACATCATCAGGCTTGGAATGAATTCTCT
CTAAAAATAAAATGATGTATGATTTGTTGTTGGCATCCCCTTTATTAATTCATTAAATTT
CTGGATTTGGGTTGTGACCCAGGGTGCATTAACTTAAAAGATTCACTAAAGCAGCACATA
GCACTGGGAACTCTGGCTCCGAAAAACTTTGTTATATATATCAAGGATGTTCTGGCTTTA
CATTTTATTTATTAGCTGTAAATACATGTGTGGATGTGTAAATGGAGCTTGTACATATTG
GAAAGGTCATTGTGGCTATCTGCATTTATAAATGTGTGGTGCTAACTGTATGTGTCTTTA
TCAGTGATGGTCTCACAGAGCCAACTCACTCTTATGAAATGGGCTTTAACAAAACAAGAA
AGAAACGTACTTAACTGTGTGAAGAAATGGAATCAGCTTTTAATAAAATTGACAACATTT
TATTACCAC

As used herein, the term “MEF2C” refers to the gene encoding Myocyte-specific enhancer factor 2C. The terms “MEF2C” and “Myocyte-specific enhancer factor 2C” include wild-type forms of the MEF2C gene, as well as variants (e.g., splice variants and polymorphisms) of wild-type MEF2C. Examples of such variants are nucleic acids having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to a wild-type MEF2C nucleic acid sequence (e.g., SEQ ID NO: 52, ENA accession number L08895). SEQ ID NO: 52 is a wild-type gene sequence encoding MEF2C protein, and is shown below:

(SEQ ID NO: 52)
GAATTCCCAGCTCTCTGCTCGCTCTGCTCGCAGTCACAGACACTTGAGCACACGCGTACA
CCCAGACATCTTCGGGCTGCTATTGGATTGACTTTGAAGGTTCTGTGTGGGTCGCCGTGG
CTGCATGTTTGAATCAGGTGGAGAAGCACTTCAACGCTGGACGAAGTAAAGATTATTGTT
GTTATTTTTTTTTTCTCTCTCTCTCTCTOTTAAGAAAGGAAAATATCCCAAGGACTAATC
TGATCGGGTCTTCCTTCATCAGGAACGAATGCAGGAATTTGGGAACTGAGCTGTGCAAGT
GCTGAAGAAGGAGATTTGTTTGGAGGAAACAGGAAAGAGAAAGAAAAGGAAGGAAAAAAT
ACATAATTTCAGGGACGAGAGAGAGAAGAAAAACGGGGACTATGGGGAGAAAAAAGATTC
AGATTACGAGGATTATGGATGAACGTAACAGACAGGTGACATTTACAAAGAGGAAATTTG
GGTTGATGAAGAAGGCTTATGAGCTGAGCGTGCTGTGTGACTGTGAGATTGCGCTGATCA
TCTTCAACAGCACCAACAAGCTGTTCCAGTATGCCAGCACCGACATGGACAAAGTGCTTC
TCAAGTACACGGAGTACAACGAGCCGCATGAGAGCCGGACAAACTCAGACATCGTGGAGA
CGTTGAGAAAGAAGGGCCTTAATGGCTGTGACAGCCCAGACCCCGATGCGGACGATTCCG
TAGGTCACAGCCCTGAGTCTGAGGACAAGTACAGGAAAATTAACGAAGATATTGATCTAA
TGATCAGCAGGCAAAGATTGTGTGCTGTTCCACCTCCCAACTTCGAGATGCCAGTCTCCA
TCCCAGTGTCCAGCCACAACAGTTTGGTGTACAGCAACCCTGTCAGCTCACTGGGAAACC
CCAACCTATTGCCACTGGCTCACCCTTCTCTGCAGAGGAATAGTATGTCTCCTGGTGTAA
CACATCGACCTCCAAGTGCAGGTAACACAGGTGGTCTGATGGGTGGAGACCTCACGTCTG
GTGCAGGCACCAGTGCAGGGAACGGGTATGGCAATCCCCGAAACTCACCAGGTCTGCTGG
TCTCACCTGGTAACTTGAACAAGAATATGCAAGCAAAATCTCCTCCCCCAATGAATTTAG
GAATGAATAACCGTAAACCAGATCTCCGAGTTCTTATTCCACCAGGCAGCAAGAATACGA
TGCCATCAGTGTCTGAGGATGTCGACCTGCTTTTGAATCAAAGGATAAATAACTCCCAGT
CGGCTCAGTCATTGGCTACCCCAGTGGTTTCCGTAGCAACTCCTACTTTACCAGGACAAG
GAATGGGAGGATATCCATCAGCCATTTCAACAACATATGGTACCGAGTACTCTCTGAGTA
GTGCAGACCTGTCATCTCTGTCTGGGTTTAACACCGCCAGCGCTCTTCACCTTGGTTCAG
TAACTGGCTGGCAACAGCAACACCTACATAACATGCCACCATCTGCCCTCAGTCAGTTGG
GAGCTTGCACTAGCACTCATTTATCTCAGAGTTCAAATCTCTCCCTGCCTTCTACTCAAA
GCCTCAACATCAAGTCAGAACCTGTTTCTCCTCCTAGAGACCGTACCACCACCCCTTCGA
GATACCCACAACACACGCGCCACGAGGCGGGGAGATCTCCTGTTGACAGCTTGAGCAGCT
GTAGCAGTTCGTACGACGGGAGCGACCGAGAGGATCACCGGAACGAATTCCACTCCCCCA
TTGGACTCACCAGACCTTCGCCGGACGAAAGGGAAAGTCCCTCAGTCAAGCGCATGCGAC
TTTCTGAAGGATGGGCAACATGATCAGATTATTACTTACTAGTTTTTTTTTTTTTCTTGC
AGTGTGTGTGTGTGCTATACCTTAATGGGGAAGGGGGGTCGATATGCATTATATGTGCCG
TGTGTGGAAAAAAAAAAAGTCAGGTACTCTGTTTTGTAAAAGTACTTTTAAATTGCCTCA
GTGATACAGTATAAAGATAAACAGAAATGCTGAGATAAGCTTAGCACTTGAGTTGTACAA
CAGAACACTTGTACAAAATAGATTTTAAGGCTAACTTCTTTTCACTGTTGTGCTCCTTTG
CAAAATGTATGTTACAATAGATAGTGTCATGTTGCAGGTTCAACGTTATTTACATGTAAA
TAGACAAAAGGAAACATTTGCCAAAAGCGGCAGATCTTTACTGAAAGAGAGAGCAGCTGT
TATGCAACATATAGAAAAATGTATAGATGCTTGGACAGACCCGGTAATGGGTGGCCATTG
GTAAATGTTAGGAACACACCAGGTCACCTGACATCCCAAGAATGCTCACAAACCTGCAGG
CATATCATTGGCGTATGGCACTCATTAAAAAGGATCAGAGACCATTAAAAGAGGACCATA
CCTATTAAAAAAAAATGTGGAGTTGGAGGGCTAACATATTTAATTAAATAAATAAATAAA
TCTGGGTCTGCATCTCTTATTAAATAAAAATATAAAAATATGTACATTACATTTTGCTTA
TTTTCATATAAAAGGTAAGACAGAGTTTGCAAAGCATTTGTGGCTTTTTGTAGTTTACTT
AAGCCAAAATGTGTTTTTTTCCCCTTGATAGCTTCGCTAATATTTTAAACAGTCCTGTAA
AAAACCAAAAAGGACTTTTTGTATAGAAAGCACTACCCTAAGCCATGAAGAACTCCATGC
TTTGCTAACCAAGATAACTGTTTTCTCTTTGTAGAAGTTTTGTTTTTGAAATGTGTATTT
CTAATTATATAAAATATTAAGAATCTTTTAAAAAAATCTGTGAAATTAACATGCTTGTGT
ATAGCTTTCTAATATATATAATATTATGGTAATAGCAGAAGTTTTGTTATCTTAATAGCG
GGAGGGGGGTATATTTGTGCAGTTGCACATTTGAGTAACTATTTTCTTTCTGTTTTCTTT
TACTCTGCTTACATTTTATAAGTTTAAGGTCAGCTGTCAAAAGGATAACCTGTGGGGTTA
GAACATATCACATTGCAACACCCTAAATTGTTTTTAATACATTAGCAATCTATTGGGTCA
ACTGACATCCATTGTATATACTAGTTTCTTTCATGCTATTTTTATTTTGTTTTTTGCATT
TTTATCAAATGCAGGGCCCCTTTCTGATCTCACCATTTCACCATGCATCTTGGAATTCAG
TAAGTGCATATCCTAACTTGCCCATATTCTAAATCATCTGGTTGGTTTTCAGCCTAGAAT
TTGATACGCTTTTTAGAAATATGCCCAGAATAGAAAAGCTATGTTGGGGCACATGTCCTG
CAAATATGGCCCTAGAAACAAGTGATATGGAATTTACTTGGTGAATAAGTTATAAATTCC
CACAGAAGAAAAATGTGAAAGACTGGGTGCTAGACAAGAAGGAAGCAGGTAAAGGGATAG
TTGCTTTGTCATCCGTTTTTAATTATTTTAACTGACCCTTGACAATCTTGTCAGCAATAT
AGGACTGTTGAACAATCCCGGTGTGTCAGGACCCCCAAATGTCACTTCTGCATAAAGCAT
GTATGTCATCTATTTTTTCTTCAATAAAGAGATTTAATAGCCATTTCAAGAAATCCCATA
AAGAACCTCTCTATGTCCCTTTTTTTAATTTAAAAAAATGACTCTTGTCTAATATTCGTC
TATAAGGGATTAATTTTCAGACCCTTTAATAAGTGAGTGCCATAAGAAAGTCAATATATA
TTGTTTAAAAGATATTTCAGTCTAGGAAAGATTTTCCTTCTCTTGGAATGTGAAGATCTG
TCGATTCATCTCCAATCATATGCATTGACATACACAGCAAAGAAGATATAGGCAGTAATA
TCAACACTGCTATATCATGTGTAGGACATTTCTTATCCATTTTTTCTCTTTTACTTGCAT
AGTTGCTATGTGTTTCTCATTGTAAAAGGCTGCCGCTGGGTGGCAGAAGCCAAGAGACCT
TATTAACTAGGCTATATTTTTCTTAACTTGATCTGAAATCCACAATTAGACCACAATGCA
CCTTTGGTTGTATCCATAAAGGATGCTAGCCTGCCTTGTACTAATGTTTTATATATT

As used herein, the term “MMP12” refers to the gene encoding Macrophage metalloelastase. The terms “MMP12” and “Macrophage metalloelastase” include wild-type forms of the MMP12 gene, as well as variants (e.g., splice variants and polymorphisms) of wild-type MMP12. Examples of such variants are nucleic acids having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to a wild-type MMP12 nucleic acid sequence (e.g., SEQ ID NO: 53, ENA accession number L23808). SEQ ID NO: 53 is a wild-type gene sequence encoding MMP12 protein, and is shown below:

(SEQ ID NO: 53)
TAGAAGTTTACAATGAAGTTTCTTCTAATACTGCTCCTGCAGGCCACTGCTTCTGGAGCT
CTTCCCCTGAACAGCTCTACAAGCCTGGAAAAAAATAATGTGCTATTTGGTGAGAGATAC
TTAGAAAAATTTTATGGCCTTGAGATAAACAAACTTCCAGTGACAAAAATGAAATATAGT
GGAAACTTAATGAAGGAAAAAATCCAAGAAATGCAGCACTTCTTGGGTCTGAAAGTGACC
GGGCAACTGGACACATCTACCCTGGAGATGATGCACGCACCTCGATGTGGAGTCCCCGAT
CTCCATCATTTCAGGGAAATGCCAGGGGGGCCCGTATGGAGGAAACATTATATCACCTAC
AGAATCAATAATTACACACCTGACATGAACCGTGAGGATGTTGACTACGCAATCCGGAAA
GCTTTCCAAGTATGGAGTAATGTTACCCCCTTGAAATTCAGCAAGATTAACACAGGCATG
GCTGACATTTTGGTGGTTTTTGCCCGTGGAGCTCATGGAGACTTCCATGCTTTTGATGGC
AAAGGTGGAATCCTAGCCCATGCTTTTGGACCTGGATCTGGCATTGGAGGGGATGCACAT
TTCGATGAGGACGAATTCTGGACTACACATTCAGGAGGCACAAACTTGTTCCTCACTGCT
GTTCACGAGATTGGCCATTCCTTAGGTCTTGGCCATTCTAGTGATCCAAAGGCTGTAATG
TTCCCCACCTACAAATATGTCGACATCAACACATTTCGCCTCTCTGCTGATGACATACGT
GGCATTCAGTCCCTGTATGGAGACCCAAAAGAGAACCAACGCTTGCCAAATCCTGACAAT
TCAGAACCAGCTCTCTGTGACCCCAATTTGAGTTTTGATGCTGTCACTACCGTGGGAAAT
AAGATCTTTTTCTTCAAAGACAGGTTCTTCTGGCTGAAGGTTTCTGAGAGACCAAAGACC
AGTGTTAATTTAATTTCTTCCTTATGGCCAACCTTGCCATCTGGCATTGAAGCTGCTTAT
GAAATTGAAGCCAGAAATCAAGTTTTTCTTTTTAAAGATGACAAATACTGGTTAATTAGC
AATTTAAGACCAGAGCCAAATTATCCCAAGAGCATACATTCTTTTGGTTTTCCTAACTTT
GTGAAAAAAATTGATGCAGCTGTTTTTAACCCACGTTTTTATAGGACCTACTTCTTTGTA
GATAACCAGTATTGGAGGTATGATGAAAGGAGACAGATGATGGACCCTGGTTATCCCAAA
CTGATTACCAAGAACTTCCAAGGAATCGGGCCTAAAATTGATGCAGTCTTCTATTCTAAA
AACAAATACTACTATTTCTTCCAAGGATCTAACCAATTTGAATATGACTTCCTACTCCAA
CGTATCACCAAAACACTGAAAAGCAATAGCTGGTTTGGTTGTTAGAAATGGTGTAATTAA
TGGTTTTTGTTAGTTCACTTCAGCTTAATAAGTATTTATTGCATATTTGCTATGTCCTCA
GTGTACCACTACTTAGAGATATGTATCATAAAAATAAAATCTGTAAACCATAGGTAATGA
TTATATAAAATACATAATATTTTTCAATTTTGAAAACTCTAATTGTCCATTCTTGCTTGA
CTCTACTATTAAGTTTGAAAATAGTTACCTTCAAAGCAAGATAATTCTATTTGAAGCATG
CTCTGTAAGTTGCTTCCTAACATCCTTGGACTGAGAAATTATACTTACTTCTGGCATAAC
TAAAATTAAGTATATATATTTTGGCTCAAATAAAATTG

As used herein, the term “MS4A4A” refers to the gene encoding Membrane Spanning 4-Domains A4A. The terms “MS4A4A” and “Membrane Spanning 4-Domains A4A” include wild-type forms of the MS4A4A gene, as well as variants (e.g., splice variants and polymorphisms) of wild-type MS4A4A. Examples of such variants are nucleic acids having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to a wild-type MS4A4A nucleic acid sequence (e.g., SEQ ID NO: 54, NCBI Reference Sequence: NM_148975.2). SEQ ID NO: 54 is a wild-type gene sequence encoding MS4A4A protein, and is shown below:

(SEQ ID NO: 54)
ATTCTCAGCACAGCCTTTAAGGTTCCAAACATCTGCTAGAAGAGGAATGCAGATTTAAACTGAGTGAG
GTGTGGAGTGGGGGAAGTTGATTGGGTCTAGACCAAAGAACTTTGAGGAACTTGCCCAGAGCCCTG
CATGCATCAGACCTACAGCAGACATTGCAGGCCTGAAGAAAGCACCTTTTCTGCTGCCATGACAACC
ATGCAAGGAATGGAACAGGCCATGCCAGGGGCTGGCCCTGGTGTGCCCCAGCTGGGAAACATGGC
TGTCATACATTCACATCTGTGGAAAGGATTGCAAGAGAAGTTCTTGAAGGGAGAACCCAAAGTCCTT
GGGGTTGTGCAGATTCTGACTGCCCTGATGAGCCTTAGCATGGGAATAACAATGATGTGTATGGCAT
CTAATACTTATGGAAGTAACCCTATTTCCGTGTATATCGGGTACACAATTTGGGGGTCAGTAATGTTT
ATTATTTCAGGATCCTTGTCAATTGCAGCAGGAATTAGAACTACAAAAGGCCTGGTCCGAGGTAGTCT
AGGAATGAATATCACCAGCTCTGTACTGGCTGCATCAGGGATCTTAATCAACACATTTAGCTTGGCGT
TTTATTCATTCCATCACCCTTACTGTAACTACTATGGCAACTCAAATAATTGTCATGGGACTATGTCCA
TCTTAATGGGTCTGGATGGCATGGTGCTCCTCTTAAGTGTGCTGGAATTCTGCATTGCTGTGTCCCT
CTCTGCCTTTGGATGTAAAGTGCTCTGTTGTACCCCTGGTGGGGTTGTGTTAATTCTGCCATCACATT
CTCACATGGCAGAAACAGCATCTCCCACACCACTTAATGAGGTTTGAGGCCACCAAAAGATCAACAG
ACAAATGCTCCAGAAATCTATGCTGACTGTGACACAAGAGCCTCACATGAGAAATTACCAGTATCCAA
CTTCGATACTGATAGACTTGTTGATATTATTATTATATGTAATCCAATTATGAACTGTGTGTGTATAGA
GAGATAATAAATTCAAAATTATGTTCTCATTTTTTTCCCTGGAACTCAATAACTCATTTCACTGGCTCTT
TATCGAGAGTACTAGAAGTTAAATTAATAAATAATGCATTTAATGAGGCAACAGCACTTGAAAGTTTTT
CATTCATCATAAGAACTTTATATAAAGGCATTACATTGGCAAATAAGGTTTGGAAGCAGAAGAGCAAA
AAAAAGATATTGTTAAAATGAGGCCTCCATGCAAAACACATACTTCCCTCCCATTTATTTAACTTTTTTT
TTCTCCTACCTATGGGGACCAAAGTGCTTTTTCCTTCAGGAAGTGGAGATGCATGGCCATCTCCCCC
TCCCTTTTTCCTTCTCCTGCTTTTCTTTCCCCATAGAAAGTACCTTGAAGTAGCACAGTCCGTCCTTG
CATGTGCACGAGCTATCATTTGAGTAAAAGTATACATGGAGTAAAAATCATATTAAGCATCAGATTCA
ACTTATATTTTCTATTTCATCTTCTTCCTTTCCCTTCTCCCACCTTCTACTGGGCATAATTATATCTTAA
TCATATATGGAAATGTGCAACATATGGTATTTGTTAAATACGTTTGTTTTTATTGCAGAGCAAAAATAA
ATCAAATTAGAAGCAATAAAAAAAAAAAAAAAAAAAA

As used herein, the term “MS4A6A” refers to the gene encoding Membrane-spanning 4-domains subfamily A member 6A. The terms “MS4A6A” and “Membrane-spanning 4-domains subfamily A member 6A” include wild-type forms of the MS4A6A gene, as well as variants (e.g., splice variants and polymorphisms) of wild-type MS4A6A. Examples of such variants are nucleic acids having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to a wild-type MS4A6A nucleic acid sequence (e.g., SEQ ID NO: 55, ENA accession number AB013104). SEQ ID NO: 55 is a wild-type gene sequence encoding MS4A6A protein, and is shown below:

(SEQ ID NO: 55)
GAGAACCAGAGTTAAAACCTCTTTGGAGCTTCTGAGGACTCAGCTGGAACCAACGGGCAC
AGTTGGCAACACCATCATGACATCACAACCTGTTCCCAATGAGACCATCATAGTGCTCCC
ATCAAATGTCATCAACTTCTCCCAAGCAGAGAAACCCGAACCCACCAACCAGGGGCAGGA
TAGCCTGAAGAAACATCTACACGCAGAAATCAAAGTTATTGGGACTATCCAGATCTTGTG
TGGCATGATGGTATTGAGCTTGGGGATCATTTTGGCATCTGCTTCCTTCTCTCCAAATTT
TACCCAAGTGACTTCTACACTGTTGAACTCTGCTTACCCATTCATAGGACCCTTTTTTTT
TATCATCTCTGGCTCTCTATCAATCGCCACAGAGAAAAGGTTAACCAAGCTTTTGGTGCA
TAGCAGCCTGGTTGGAAGCATTCTGAGTGCTCTGTCTGCCCTGGTGGGTTTCATTATCCT
GTCTGTCAAACAGGCCACCTTAAATCCTGCCTCACTGCAGTGGAACTCTCTCTCTGATGC
TGATTTGCACTCTGCTGGAATTCTGCCTAGCTGTGCTCACTGCTGTGCTGCGGTGGAAAC
AGGCTTACTCTGACTTCCCTGGGAGTGGACTTTTCCTGCCTCACAGTTACATTGGTAATT
CTGGCATGTCCTCAAAAATGACTCATGACTGTGGATATGAAGAACTATTGACTTCTTAAG
AAAAAAGGGAGAAATATTAATCAGAAAGTTGATTCTTATGATAATATGGAAAAGTTAACC
ATTATAGAAAAGCAAAGCTTGAGTTTCCTAAATGTAAGCTTTTAAAGTAATGAACATTAA
AAAAAACCATTATTTCACTGTC

As used herein, the term “NLRP3” refers to the gene encoding NACHT, LRR and PYD domains-containing protein 3. The terms “NLRP3” and “NACHT, LRR and PYD domains-containing protein 3” include wild-type forms of the NLRP3 gene, as well as variants (e.g., splice variants and polymorphisms) of wild-type NLRP3. Examples of such variants are nucleic acids having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to a wild-type NLRP3 nucleic acid sequence (e.g., SEQ ID NO: 56, ENA accession number AF410477). SEQ ID NO: 56 is a wild-type gene sequence encoding NLRP3 protein, and is shown below:

(SEQ ID NO: 56)
GTAGATGAGGAAACTGAAGTTGAGGAATAGTGAAGAGTTTGTCCAATGTCATAGCCCCGT
AATCAACGGGACAAAAATTTTCTTGCTGATGGGTCAAGATGGCATCGTGAAGTGGTTGTT
CACCGTAAACTGTAATACAATCCTGTTTATGGATTTGTTTGCATATTTTTCCCCCCATAG
GGAAACCTTTTTTCCATGGCTCAGGACACACTCCTGGATCGAGCCAACAGGAGAACTTTC
TGGTAAGCATTTGGCTAACTTTTTTTTTTTTGAGATGGAGTCTTGCTGTGTCGCCTAGGC
TGGAGTGCAGTGGCGTGATCTTGGCTCACTGCAGCCTCCACCTCCCGGGTTCAATCAATT
CTCCTACCTCAACTTCCTGAGTAGCTGGGATTACAGGCGCCCGCCACCACACCCGGCTCA
TTTTTGTACTTTTAGTAGAGACACAGTTTTGCCATGTTGGCCAGGCTGGTCTTGAATTCC
TCAGCTCAGGTGATATGCCTGCCTTGGCCTCTCAAAGTGCTGGGATTACAGGCGTGAGCC
ACTGTGCCCGGCCTTGGCTAACTTTTCAAAATTAAAGATTTTGACTTGTTACAGTCATGT
GACATTTTTTTCTTTCTGTTTGGTGAGTTTTTGATAATTTATATCTCTCAAAGTGGAGAC
TTTAAAAAAGACTCATCTGTGTGCCGTGTTCACTGCCTGGTATCTTAGTGTGGACCGAAG
CCTAAGGACCCTGAAAACAGCTGCAGATGAAGATGGCAAGCACCCGCTGCAAGCTGGCCA
GGTACCTGGAGGACCTGGAGGATGTGGACTTGAAGAAATTTAAGATGCACTTAGAGGACT
ATCCTCCCCAGAAGGGCTGCATCCCCCTCCCGAGGGGTCAGACAGAGAAGGCAGACCATG
TGGATCTAGCCACGCTAATGATCGACTTCAATGGGGAGGAGAAGGCGTGGGCCATGGCCG
TGTGGATCTTCGCTGCGATCAACAGGAGAGACCTTTATGAGAAAGCAAAAAGAGATGAGC
CGAAGTGGGGTTCAGATAATGCACGTGTTTCGAATCCCACTGTGATATGCCAGGAAGACA
GCATTGAAGAGGAGTGGATGGGTTTACTGGAGTACCTTTCGAGAATCTCTATTTGTAAAA
TGAAGAAAGATTACCGTAAGAAGTACAGAAAGTACGTGAGAAGCAGATTCCAGTGCATTG
AAGACAGGAATGCCCGTCTGGGTGAGAGTGTGAGCCTCAACAAACGCTACACACGACTGC
GTCTCATCAAGGAGCACCGGAGCCAGCAGGAGAGGGAGCAGGAGCTTCTGGCCATCGGCA
AGACCAAGACGTGTGAGAGCCCCGTGAGTCCCATTAAGATGGAGTTGCTGTTTGACCCCG
ATGATGAGCATTCTGAGCCTGTGCACACCGTGGTGTTCCAGGGGGGGGCAGGGATTGGGA
AAACAATCCTGGCCAGGAAGATGATGTTGGACTGGGCGTCGGGGACACTCTACCAAGACA
GGTTTGACTATCTGTTCTATATCCACTGTCGGGAGGTGAGCCTTGTGACACAGAGGAGCC
TGGGGGACCTGATCATGAGCTGCTGCCCCGACCCAAACCCACCCATCCACAAGATCGTGA
GAAAACCCTCCAGAATCCTCTTCCTCATGGACGGCTTCGATGAGCTGCAAGGTGCCTTTG
ACGAGCACATAGGACCGCTCTGCACTGACTGGCAGAAGGCCGAGCGGGGAGACATTCTCC
TGAGCAGCCTCATCAGAAAGAAGCTGCTTCCCGAGGCCTCTCTGCTCATCACCACGAGAC
CTGTGGCCCTGGAGAAACTGCAGCACTTGCTGGACCATCCTCGGCATGTGGAGATCCTGG
GTTTCTCCGAGGCCAAAAGGAAAGAGTACTTCTTCAAGTACTTCTCTGATGAGGCCCAAG
CCAGGGCAGCCTTCAGTCTGATTCAGGAGAACGAGGTCCTCTTCACCATGTGCTTCATCC
CCCTGGTCTGCTGGATCGTGTGCACTGGACTGAAACAGCAGATGGAGAGTGGCAAGAGCC
TTGCCCAGACATCCAAGACCACCACCGCGGTGTACGTCTTCTTCCTTTCCAGTTTGCTGC
AGCCCCGGGGAGGGAGCCAGGAGCACGGCCTCTGCGCCCACCTCTGGGGGCTCTGCTCTT
TGGCTGCAGATGGAATCTGGAACCAGAAAATCCTGTTTGAGGAGTCCGACCTCAGGAATC
ATGGACTGCAGAAGGCGGATGTGTCTGCTTTCCTGAGGATGAACCTGTTCCAAAAGGAAG
TGGACTGCGAGAAGTTCTACAGCTTCATCCACATGACTTTCCAGGAGTTCTTTGCCGCCA
TGTACTACCTGCTGGAAGAGGAAAAGGAAGGAAGGACGAACGTTCCAGGGAGTCGTTTGA
AGCTTCCCAGCCGAGACGTGACAGTCCTTCTGGAAAACTATGGCAAATTCGAAAAGGGGT
ATTTGATTTTTGTTGTACGTTTCCTCTTTGGCCTGGTAAACCAGGAGAGGACCTCCTACT
TGGAGAAGAAATTAAGTTGCAAGATCTCTCAGCAAATCAGGCTGGAGCTGCTGAAATGGA
TTGAAGTGAAAGCCAAAGCTAAAAAGCTGCAGATCCAGCCCAGCCAGCTGGAATTGTTCT
ACTGTTTGTACGAGATGCAGGAGGAGGACTTCGTGCAAAGGGCCATGGACTATTTCCCCA
AGATTGAGATCAATCTCTCCACCAGAATGGACCACATGGTTTCTTCCTTTTGCATTGAGA
ACTGTCATCGGGTGGAGTCACTGTCCCTGGGGTTTCTCCATAACATGCCCAAGGAGGAAG
AGGAGGAGGAAAAGGAAGGCCGACACCTTGATATGGTGCAGTGTGTCCTCCCAAGCTCCT
CTCATGCTGCCTGTTCTCATGGATTGGTGAACAGCCACCTCACTTCCAGTTTTTGCCGGG
GCCTCTTTTCAGTTCTGAGCACCAGCCAGAGTCTAACTGAATTGGACCTCAGTGACAATT
CTCTGGGGGACCCAGGGATGAGAGTGTTGTGTGAAACGCTCCAGCATCCTGGCTGTAACA
TTCGGAGATTGTGGTTGGGGCGCTGTGGCCTCTCGCATGAGTGCTGCTTCGACATCTCCT
TGGTCCTCAGCAGCAACCAGAAGCTGGTGGAGCTGGACCTGAGTGACAACGCCCTCGGTG
ACTTCGGAATCAGACTTCTGTGTGTGGGACTGAAGCACCTGTTGTGCAATCTGAAGAAGC
TCTGGTTGGTCAGCTGCTGCCTCACATCAGCATGTTGTCAGGATCTTGCATCAGTATTGA
GCACCAGCCATTCCCTGACCAGACTCTATGTGGGGGAGAATGCCTTGGGAGACTCAGGAG
TCGCAATTTTATGTGAAAAAGCCAAGAATCCACAGTGTAACCTGCAGAAACTGGGGTTGG
TGAATTCTGGCCTTACGTCAGTCTGTTGTTCAGCTTTGTCCTCGGTACTCAGCACTAATC
AGAATCTCACGCACCTTTACCTGCGAGGCAACACTCTCGGAGACAAGGGGATCAAACTAC
TCTGTGAGGGACTCTTGCACCCCGACTGCAAGCTTCAGGTGTTGGAATTAGACAACTGCA
ACCTCACGTCACACTGCTGCTGGGATCTTTCCACACTTCTGACCTCCAGCCAGAGCCTGC
GAAAGCTGAGCCTGGGCAACAATGACCTGGGCGACCTGGGGGTCATGATGTTCTGTGAAG
TGCTGAAACAGCAGAGCTGCCTCCTGCAGAACCTGGGGTTGTCTGAAATGTATTTCAATT
ATGAGACAAAAAGTGCGTTAGAAACACTTCAAGAAGAAAAGCCTGAGCTGACCGTCGTCT
TTGAGCCTTCTTGGTAGGAGTGGAAACGGGGCTGCCAGACGCCAGTGTTCTCCGGTCCCT
CCAGCTGGGGGCCCTCAGGTGGAGAGAGCTGCGATCCATCCAGGCCAAGACCACAGCTCT
GTGATCCTTCCGGTGGAGTGTCGGAGAAGAGAGCTTGCCGACGATGCCTTCCTGTGCAGA
GCTTGGGCATCTCCTTTACGCCAGGGTGAGGAAGACACCAGGACAATGACAGCATCGGGT
GTTGTTGTCATCACAGCGCCTCAGTTAGAGGATGTTCCTCTTGGTGACCTCATGTAATTA
GCTCATTCAATAAAGCACTTTCTTTATTTT

As used herein, the term “NME8” refers to the gene encoding Thioredoxin domain-containing protein 3. The terms “NME8” and “Thioredoxin domain-containing protein 3” include wild-type forms of the NME8 gene, as well as variants (e.g., splice variants and polymorphisms) of wild-type NME8. Examples of such variants are nucleic acids having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to a wild-type NME8 nucleic acid sequence (e.g., SEQ ID NO: 57, ENA accession number AF202051). SEQ ID NO: 57 is a wild-type gene sequence encoding NME8 protein, and is shown below:

(SEQ ID NO: 57)
CGGCCACAACGAGGGAGCCGATTTAGATCCTCTGGGCCTGTTCCTTCCTTTTCTTTAAAC
GTCCCAGTCTAGCTTAGAGGAGGACCTGTTTTGTTAGATAAATGGCAAGCAAAAAACGAG
AAGTCCAGTTACAGACAGTCATCAATAATCAAAGCCTGTGGGATGAGATGTTGCAGAACA
AAGGCTTAACAGTGATTGATGTTTACCAAGCCTGGTGTGGACCTTGCAGAGCAATGCAAC
CTTTATTCAGAAAATTGAAAAATGAACTGAACGAAGACGAAATTCTGCATTTTGCTGTCG
CAGAAGCTGACAACATTGTGACTTTGCAGCCATTTAGAGATAAATGTGAACCTGTTTTTC
TCTTTAGTGTTAATGGCAAAATTATCGAAAAGATTCAGGGTGCAAATGCACCGCTTGTTA
ATAAAAAAGTTATTAATTTGATCGATGAGGAGAGAAAAATTGCAGCAGGTGAAATGGCTC
GACCTCAGTATCCTGAAATTCCATTAGTAGACTCAGATTCAGAAGTTAGTGAAGAATCAC
CATGTGAAAGTGTTCAGGAATTATACAGTATTGCTATTATCAAACCGGATGCTGTGATTA
GTAAAAAAGTTCTAGAAATTAAAAGAAAAATTACCAAAGCTGGATTTATTATAGAAGCAG
AGCATAAGACAGTGCTCACTGAAGAACAAGTTGTCAACTTCTATAGTCGAATAGCAGACC
AGTGTGACTTCGAAGAGTTTGTCTCTTTTATGACAAGTGGCTTAAGCTATATTCTAGTTG
TATCTCAAGGAAGTAAACACAATCCTCCCTCTGAAGAAACCGAACCACAGACTGACACCG
AACCTAACGAACGATCTGAGGATCAACCTGAGGTCGAAGCCCAGGTTACACCTGGAATGA
TGAAGAACAAACAAGACAGTTTACAAGAATATCTGGAAAGACAACATTTAGCTCAGCTCT
GTGACATTGAAGAGGATGCAGCTAATGTTGCTAAGTTCATGGATGCTTTCTTCCCCGATT
TTAAAAAAATGAAAAGCATGAAATTAGAAAAGACATTGGCATTACTTCGACCAAATCTCT
TTCATGAAAGGAAAGATGATGTTTTGCGTATTATTAAAGATGAAGACTTCAAAATACTGG
AGCAAAGACAAGTAGTATTATCGGAAAAAGAAGCACAAGCACTGTGCAAGGAATATGAAA
ATGAAGACTATTTTAATAAACTTATAGAAAACATGACCAGTGGTCCATCTCTAGCCCTTG
TTTTATTGAGAGACAATGGCTTGCAATACTGGAAACAATTACTGGGACCAAGAACTGTTG
AAGAAGCCATTGAATATTTTCCAGAGAGTTTATGTGCACAGTTTGCGATGGACAGTTTGC
CGGTCAACCAGTTGTATGGCAGCGATTCATTAGAAACCGCTGAAAGGGAAATACAGCATT
TCTTTCCTCTTCAAAGCACTTTAGGCTTGATTAAACCTCATGCAACAAGTGAACAAAGAG
AGCAGATCCTGAAGATAGTTAAGGAGGCTGGATTTGATCTGACACAGGTGAAGAAAATGT
TCCTAACTCCTGAGCAAATAGAGAAAATTTATCCAAAAGTAACAGGAAAAGACTTTTATA
AAGATTTATTGGAAATGTTATCTGTGGGTCCATCTATGGTCATGATTCTGACCAAGTGGA
ATGCTGTTGCAGAATGGAGACGATTGATGGGCCCAACAGACCCAGAAGAAGCAAAATTAC
TTTCCCCTGACTCCATCCGAGCCCAGTTTGGAATAAGTAAATTGAAAAACATTGTCCATG
GAGCATCTAACGCCTATGAAGCAAAAGAGGTTGTTAATAGACTCTTTGAGGATCCTGAGG
AAAACTAAAGTATATACTGTGAAAACTTTGAGAAGATAATACATATGTTCACGTCAATAT
ACAACCATTTGGCACAGCTTCCTGGGAGGAATAATAAGAAAAACATGCTTTGGAGGAAAA
CTCAAGATACAAAAATGAATGGCTATGCATAATAACAATAAAAATGTATTCCCCAAAC

As used herein, the term “NOS2” refers to the gene encoding Nitric oxide synthase, inducible. The terms “NOS2” and “Nitric oxide synthase, inducible” include wild-type forms of the NOS2 gene, as well as variants (e.g., splice variants and polymorphisms) of wild-type NOS2. Examples of such variants are nucleic acids having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to a wild-type NOS2 nucleic acid sequence (e.g., SEQ ID NO: 58, ENA accession number L24553). SEQ ID NO: 58 is a wild-type gene sequence encoding NOS2 protein, and is shown below:

(SEQ ID NO: 58)
AAGCCCCACAGTGAAGAACATCTGAGCTCAAATCCAGATAAGTGACATAAGTGACCTGCT
TTGTAAAGCCATAGAGATGGCCTGTCCTTGGAAATTTCTGTTCAAGACCAAATTCCACCA
GTATGCAATGAATGGGGAAAAAGACATCAACAACAATGTGGAGAAAGCCCCCTGTGCCAC
CTCCAGTCCAGTGACACAGGATGACCTTCAGTATCACAACCTCAGCAAGCAGCAGAATGA
GTCCCCGCAGCCCCTCGTGGAGACGGGAAAGAAGTCTCCAGAATCTCTGGTCAAGCTGGA
TGCAACCCCATTGTCCTCCCCACGGCATGTGAGGATCAAAAACTGGGGCAGCGGGATGAC
TTTCCAAGACACACTTCACCATAAGGCCAAAGGGATTTTAACTTGCAGGTCCAAATCTTG
CCTGGGGTCCATTATGACTCCCAAAAGTTTGACCAGAGGACCCAGGGACAAGCCTACCCC
TCCAGATGAGCTTCTACCTCAAGCTATCGAATTTGTCAACCAATATTACGGCTCCTTCAA
AGAGGCAAAAATAGAGGAACATCTGGCCAGGGTGGAAGCGGTAACAAAGGAGATAGAAAC
AACAGGAACCTACCAACTGACGGGAGATGAGCTCATCTTCGCCACCAAGCAGGCCTGGCG
CAATGCCCCACGCTGCATTGGGAGGATCCAGTGGTCCAACCTGCAGGTCTTCGATGCCCG
CAGCTGTTCCACTGCCCGGGAAATGTTTGAACACATCTGCAGACACGTGCGTTACTCCAC
CAACAATGGCAACATCAGGTCGGCCATCACCGTGTTCCCCCAGCGGAGTGATGGCAAGCA
CGACTTCCGGGTGTGGAATGCTCAGCTCATCCGCTATGCTGGCTACCAGATGCCAGATGG
CAGCATCAGAGGGGACCCTGCCAACGTGGAATTCACTCAGCTGTGCATCGACCTGGGCTG
GAAGCCCAAGTACGGCCGCTTCGATGTGGTCCCCCTGGTCCTGCAGGCCAATGGCCGTGA
CCCTGAGCTCTTCGAAATCCCACCTGACCTTGTGCTTGAGGTGGCCATGGAACATCCCAA
ATACGAGTGGTTTCGGGAACTGGAGCTAAAGTGGTACGCCCTGCCTGCAGTGGCCAACAT
GCTGCTTGAGGTGGGCGGCCTGGAGTTCCCAGGGTGCCCCTTCAATGGCTGGTACATGGG
CACAGAGATCGGAGTCCGGGACTTCTGTGACGTCCAGCGCTACAACATCCTGGAGGAAGT
GGGCAGGAGAATGGGCCTGGAAACGCACAAGCTGGCCTCGCTCTGGAAAGACCAGGCTGT
CGTTGAGATCAACATTGCTGTGCTCCATAGTTTCCAGAAGCAGAATGTGACCATCATGGA
CCACCACTCGGCTGCAGAATCCTTCATGAAGTACATGCAGAATGAATACCGGTCCCGTGG
GGGCTGCCCGGCAGACTGGATTTGGCTGGTCCCTCCCATGTCTGGGAGCATCACCCCCGT
GTTTCACCAGGAGATGCTGAACTACGTCCTGTCCCCTTTCTACTACTATCAGGTAGAGGC
CTGGAAAACCCATGTCTGGCAGGACGAGAAGCGGAGACCCAAGAGAAGAGAGATTCCATT
GAAAGTCTTGGTCAAAGCTGTGCTCTTTGCCTGTATGCTGATGCGCAAGACAATGGCGTC
CCGAGTCAGAGTCACCATCCTCTTTGCGACAGAGACAGGAAAATCAGAGGCGCTGGCCTG
GGACCTGGGGGCCTTATTCAGCTGTGCCTTCAACCCCAAGGTTGTCTGCATGGATAAGTA
CAGGCTGAGCTGCCTGGAGGAGGAACGGCTGCTGTTGGTGGTGACCAGTACGTTTGGCAA
TGGAGACTGCCCTGGCAATGGAGAGAAACTGAAGAAATCGCTCTTCATGCTGAAAGAGCT
CAACAACAAATTCAGGTACGCTGTGTTTGGCCTCGGCTCCAGCATGTACCCTCGGTTCTG
CGCCTTTGCTCATGACATTGATCAGAAGCTGTCCCACCTGGGGGCCTCTCAGCTCACCCC
GATGGGAGAAGGGGATGAGCTCAGTGGGCAGGAGGACGCCTTCCGCAGCTGGGCCGTGCA
AACCTTCAAGGCAGCCTGTGAGACGTTTGATGTCCGAGGCAAACAGCACATTCAGATCCC
CAAGCTCTACACCTCCAATGTGACCTGGGACCCGCACCACTACAGGCTCGTGCAGGACTC
ACAGCCTTTGGACCTCAGCAAAGCCCTCAGCAGCATGCATGCCAAGAACGTGTTCACCAT
GAGGCTCAAATCTCGGCAGAATCTACAAAGTCCGACATCCAGCCGTGCCACCATCCTGGT
GGAACTCTCCTGTGAGGATGGCCAAGGCCTGAACTACCTGCCGGGGGAGCACCTTGGGGT
TTGCCCAGGCAACCAGCCGGCCCTGGTCCAAGGCATCCTGGAGCGAGTGGTGGATGGCCC
CACACCCCACCAGACAGTGCGCCTGGAGGCCCTGGATGAGAGTGGCAGCTACTGGGTCAG
TGACAAGAGGCTGCCCCCCTGCTCACTCAGCCAGGCCCTCACCTACTTCCTGGACATCAC
CACACCCCCAACCCAGCTGCTGCTCCAAAAGCTGGCCCAGGTGGCCACAGAAGAGCCTGA
GAGACAGAGGCTGGAGGCCCTGTGCCAGCCCTCAGAGTACAGCAAGTGGAAGTTCACCAA
CAGCCCCACATTCCTGGAGGTGCTAGAGGAGTTCCCGTCCCTGCGGGTGTCTGCTGGCTT
CCTGCTTTCCCAGCTCCCCATTCTGAAGCCCAGGTTCTACTCCATCAGCTCCTCCCGGGA
TCACACGCCCACGGAGATCCACCTGACTGTGGCCGTGGTCACCTACCACACCCGAGATGG
CCAGGGTCCCCTGCACCACGGCGTCTGCAGCACATGGCTCAACAGCCTGAAGCCCCAAGA
CCCAGTGCCCTGCTTTGTGCGGAATGCCAGCGGCTTCCACCTCCCCGAGGATCCCTOCCA
TCCTTGCATCCTCATCGGGCCTGGCACAGGCATCGCGCCCTTCCGCAGTTTCTGGCAGCA
ACGGCTCCATGACTCCCAGCACAAGGGAGTGCGGGGAGGCCGCATGACCTTGGTGTTTGG
GTGCCGCCGCCCAGATGAGGACCACATCTACCAGGAGGAGATGCTGGAGATGGCCCAGAA
GGGGGTGCTGCATGCGGTGCACACAGCCTATTCCCGCCTGCCTGGCAAGCCCAAGGTCTA
TGTTCAGGACATCCTGCGGCAGCAGCTGGCCAGCGAGGTGCTCCGTGTGCTCCACAAGGA
GCCAGGCCACCTCTATGTTTGCGGGGATGTGCGCATGGCCCGGGACGTGGCCCACACCCT
GAAGCAGCTGGTGGCTGCCAAGCTGAAATTGAATGAGGAGCAGGTCGAGGACTATTTCTT
TCAGCTCAAGAGCCAGAAGCGCTATCACGAAGATATCTTTGGTGCTGTATTTCCTTACGA
GGCGAAGAAGGACAGGGTGGCGGTGCAGCCCAGCAGCCTGGAGATGTCAGCGCTCTGAGG
GCCTACAGGAGGGGTTAAAGCTGCCGGCACAGAACTTAAGGATGGAGCCAGCTCT

As used herein, the term “PICALM” refers to the gene encoding Phosphatidylinositol-binding clathrin assembly protein. The terms “PICALM” and “Phosphatidylinositol-binding clathrin assembly protein” include wild-type forms of the PICALM gene, as well as variants (e.g., splice variants and polymorphisms) of wild-type PICALM. Examples of such variants are nucleic acids having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to a wild-type PICALM nucleic acid sequence (e.g., SEQ ID NO: 59, ENA accession number U45976). SEQ ID NO: 59 is a wild-type gene sequence encoding PICALM protein, and is shown below:

(SEQ ID NO: 59)
GCGCGGCCCCGAACCGCCGCCAGGCCGGCACGGGGGAAGGAGCCGGTGGGGGTAGGGGGT
GCGGTGGGGGGTGGGGACCCTCCGGCTCTTGGGGGTCCCAGTCCCCGCCGGCTGCTGAGC
GGGTGGGGTGGTGGAGGAGCTGCAGAGATGTCCGGCCAGAGCCTGACGGACCGAATCACT
GCCGCCCAGCACAGTGTCACCGGCTCTGCCGTATCCAAGACAGTATGCAAGGCCACGACC
CACGAGATCATGGGGCCCAAGAAAAAGCACCTGGACTACTTAATTCAGTGCACAAATGAG
ATGAATGTGAACATCCCACAGTTGGCAGACAGTTTATTTGAAAGAACTACTAATAGTAGT
TGGGTGGTGGTCTTCAAATCTCTCATTACAACTCATCATTTGATGGTGTATGGAAATGAG
CGTTTTATTCAGTATTTGGCTTCAAGAAACACGTTGTTTAACTTAAGCAATTTTTTGGAT
AAAAGTGGATTGCAAGGATATGACATGTCTACATTTATTAGGCGGTATAGTAGATATTTA
AATGAGAAAGCAGTTTCATACAGACAAGTTGCATTTGATTTCACAAAAGTGAAGAGAGGG
GCTGATGGAGTTATGAGAACAATGAACACAGAAAAACTCCTAAAAACTGTACCAATTATT
CAGAATCAAATGGATGCACTTCTTGATTTTAATGTTAATAGCAATGAACTTACAAATGGG
GTAATAAATGCTGCCTTCATGCTCCTGTTCAAAGATGCCATTAGACTGTTTGCAGCATAC
CATGAAGGAATTATTAATTTGTTGGAAAAATATTTTGATATGAAAAAGAACCAATGCAAA
GAAGGTCTTGACATCTATAAGAAGTTCCTAACTAGGATGACAAGAATCTCAGAGTTCCTC
AAAGTTGCAGAGCAAGTTGGAATTGACAGAGGTGATATACCAGACCTTTCACAGGCCCCT
AGCAGTCTTCTTGATGCTTTGGAACAACATTTAGCTTCCTTGGAAGGAAAGAAAATCAAA
GATTCTACAGCTGCAAGCAGGGCAACTACACTTTCCAATGCAGTGTCTTCCCTGGCAAGC
ACTGGTCTATCTCTGACCAAAGTGGATGAAAGGGAAAAGCAGGCAGCATTAGAGGAAGAA
CAGGCACGTTTGAAAGCTTTAAAGGAACAGCGCCTAAAAGAACTTGCAAAGAAACCTCAT
ACCTCTTTAACAACTGCAGCCTCTCCTGTATCCACCTCAGCAGGAGGGATAATGACTGCA
CCAGCCATTGACATATTTTCTACCCCTAGTTCTTCTAACAGCACATCAAAGCTGCCCAAT
GATCTGCTTGATTTGCAGCAGCCAACTTTTCACCCATCTGTACATCCTATGTCAACTGCT
TCTCAGGTAGCAAGTACATGGGGAGATCCTTTCTCTGCTACTGTAGATGCTGTTGATGAT
GCCATTCCAAGCTTAAATCCTTTCCTCACAAAAAGTAGTGGTGATGTTCACCTTTCCATT
TCTTCAGATGTATCTACTTTTACTACTAGGACACCTACTCATGAAATGTTTGTTGGATTC
ACTCCTTCTCCAGTTGCACAGCCACACCCTTCAGCTGGCCTTAATGTTGACTTTGAATCT
GTGTTTGGAAATAAATCTACAAATGTTATTGTAGATTCTGGGGGCTTTGATGAACTAGGT
GGACTTCTCAAACCAACAGTGGCCTCTCAGAACCAGAACCTTCCTGTTGCCAAACTCCCA
CCTAGCAAGTTAGTATCTGATGACTTGGATTCATCTTTAGCCAACCTTGTGGGCAATCTT
GGCATCGGAAATGGAACCACTAAGAATGATGTAAATTGGAGTCAACCAGGTGAAAAGAAG
TTAACTGGGGGATCTAACTGCGAACCAAAGGTTGCACCAACAACCGCTTGGAATGCTGCA
ACAATGGCACCCCCTGTAATGGCCTATCCTGCTACTACACCAACAGGCATGATAGGATAT
GGAATTCCTCCACAAATGGGAAGTGTTCCTGTAATGACGCAACCAACCTTAATATACAGC
CAGCCTGTCATGAGACCTCCAAACCCCTTTGGCCCTGTATCAGGAGCACAGATACAGTTT
ATGTAACTTGATGGAAGAAAATGGAATTACTCCAAAAAGACAAGTGCTCAAGCAGCAAAA
TCCTTACTTCCAGCAAAATCCAAACTGCTGTCTCTTAAATCTCTTAAACTCTCTTCTTCC
ATTAGGATGCTACAAGTANCTCAGTGAAGGCCCATGAAGGGAATTGGGGACTAGTTTATA
GGGNGAACGTATTCATTACAGTTTATAAAGGCCAGGATTGGNTTGGATTTTAGGATTANG
TTC

As used herein, the term “PILRA” refers to the gene encoding Paired Immunoglobin Like Type 2 Receptor Alpha. The terms “PILRA” and “Paired Immunoglobin Like Type 2 Receptor Alpha” include wild-type forms of the PILRA gene, as well as variants (e.g., splice variants and polymorphisms) of wild-type PILRA. Examples of such variants are nucleic acids having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to a wild-type PILRA nucleic acid sequence (e.g., SEQ ID NO: 60, NCBI Reference Sequence: NM_013439.2). SEQ ID NO: 60 is a wild-type gene sequence encoding PILRA protein, and is shown below:

(SEQ ID NO: 60)
AATAGGGGAAAATAAGCCAGATGGATAAAGGAAGTGCTGGTCACCCTGGAGGTGCACTGGTTTGGG
GAAGGCTCCTGGCCCCCACAGCCCTCTTCGGAGCCTGAGCCCGGCTCTCCTCACTCACCTCAACCC
CCAGGCGGCCCCTCCACAGGGCCCCTCTCCTGCCTGGACGGCTCTGCTGGTCTCCCCGTCCCCTG
GAGAAGAACAAGGCCATGGGTCGGCCCCTGCTGCTGCCCCTACTGCCCTTGCTGCTGCCGCCAGC
ATTTCTGCAGCCTAGTGGCTCCACAGGATCTGGTCCAAGCTACCTTTATGGGGTCACTCAACCAAAA
CACCTCTCAGCCTCCATGGGTGGCTCTGTGGAAATCCCCTTCTCCTTCTATTACCCCTGGGAGTTAG
CCACAGCTCCCGACGTGAGAATATCCTGGAGACGGGGCCACTTCCACAGGCAGTCCTTCTACAGCA
CAAGGCCGCCTTCCATTCACAAGGATTATGTGAACCGGCTCTTTCTGAACTGGACAGAGGGTCAGAA
GAGCGGCTTCCTCAGGATCTCCAACCTGCAGAAGCAGGACCAGTCTGTGTATTTCTGCCGAGTTGA
GCTGGACACACGGAGCTCAGGGAGGCAGCAGTGGCAGTCCATCGAGGGGACCAAACTCTCCATCA
CCCAGGCTGTCACGACCACCACCCAGAGGCCCAGCAGCATGACTACCACCTGGAGGCTCAGTAGC
ACAACCACCACAACCGGCCTCAGGGTCACACAGGGCAAACGACGCTCAGACTCTTGGCACATAAGT
CTGGAGACTGCTGTGGGGGTGGCAGTGGCTGTCACTGTGCTCGGAATCATGATTTTGGGACTGATC
TGCCTCCTCAGGTGGAGGAGAAGGAAAGGTCAGCAGCGGACTAAAGCCACAACCCCAGCCAGGGA
ACCCTTCCAAAACACAGAGGAGCCATATGAGAATATCAGGAATGAAGGACAAAATACAGATCCCAAG
CTAAATCCCAAGGATGACGGCATCGTCTATGCTTCCCTTGCCCTCTCCAGCTCCACCTCACCCAGAG
CACCTCCCAGCCACCGTCCCCTCAAGAGCCCCCAGAACGAGACCCTGTACTCTGTCTTAAAGGCCT
AACCAATGGACAGCCCTCTCAAGACTGAATGGTGAGGCCAGGTACAGTGGCGCACACCTGTAATCC
CAGCTACTCTGAAGCCTGAGGCAGAATCAAGTGAGCCCAGGAGTTCAGGGCCAGCTTTGATAATGG
AGCGAGATGCCATCTCTAGTTAAAAATATATATTAACAATAAAGTAACAAATTTAAAAAGATAAAAAAA

As used herein, the term “PLCG2” refers to the gene encoding 1-phosphatidylinositol 4,5-bisphosphate phosphodiesterase gamma-2. The terms “PLCG2” and “1-phosphatidylinositol 4,5-bisphosphate phosphodiesterase gamma-2” include wild-type forms of the PLCG2 gene, as well as variants (e.g., splice variants and polymorphisms) of wild-type PLCG2. Examples of such variants are nucleic acids having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to a wild-type PLCG2 nucleic acid sequence (e.g., SEQ ID NO: 61, ENA accession number M37238). SEQ ID NO: 61 is a wild-type gene sequence encoding PLCG2 protein, and is shown below:

(SEQ ID NO: 61)
GAATTCGGCGCTGAGTGACCCGAGTCGGGACGCGGGCTGCGCGCGCGGGACCCCGGAGCC
CAAACCCGGGGCAGGCGGGCAGCTGTGCCCGGGCGGCACGGCCAGCTTCCTGATTTCTCC
CGATTCCTTCCTTCTCCCTGGAGCGGCCGACAATGTCCACCACGGTCAATGTAGATTCCC
TTGCGGAATATGAGAAGAGCCAGATCAAGAGAGCCCTGGAGCTGGGGACGGTGATGACTG
TGTTCAGCTTCCGCAAGTCCACCCCCGAGCGGAGAACCGTCCAGGTGATCATGGAGACGC
GGCAGGTGGCCTGGAGCAAGACCGCCGACAAGATCGAGGGCTTCTTGGATATCATGGAAA
TAAAAGAAATCCGCCCAGGGAAGAACTCCAAAGATTTCGAGCGAGCAAAAGCAGTTCGCC
AGAAAGAAGACTGCTGCTTCACCATCCTATATGGCACTCAGTTCGTCCTCAGCACGCTCA
GCTTGGCAGCTGACTCTAAAGAGGATGCAGTTAACTGGCTCTCTGGCTTGAAAATCTTAC
ACCAGGAAGCGATGAATGCGTCCACGCCCACCATTATCGAGAGTTGGCTGAGAAAGCAGA
TATATTCTGTGGATCAAACCAGAAGAAACAGCATCAGTCTCCGAGAGTTGAAGACCATCT
TGCCCCTGATCAACTTTAAAGTGAGCAGTGCCAAGTTCCTTAAAGATAAGTTTGTGGAAA
TAGGAGCACACAAAGATGAGCTCAGCTTTGAACAGTTCCATCTCTTCTATAAAAAACTTA
TGTTTGAACAGCAAAAATCGATTCTCGATGAATTCAAAAAGGATTCGTCCGTGTTCATCC
TGGGGAACACTGACAGGCCGGATGCCTCTGCTGTTTACCTGCATGACTTCCAGAGGTTTC
TCATACATGAACAGCAGGAGCATTGGGCTCAGGATCTGAACAAAGTCCGTGAGCGGATGA
CAAAGTTCATTGATGACACCATGCGTGAAACTGCTGAGCCTTTCTTGTTTGTGGATGAGT
TCCTCACGTACCTGTTTTCACGAGAAAACAGCATCTGGGATGAGAAGTATGACGCGGTGG
ACATGCAGGACATGAACAACCCCCTGTCTCATTACTGGATCTCCTCGTCACATAACACGT
ACCTTACAGGTGACCAGCTGCGGAGCGAGTCGTCCCCAGAAGCTTACATCCGCTGCCTGC
GCATGGGCTGTCGCTGCATTGAACTGGACTGCTGGGACGGGCCCGATGGGAAGCCGGTCA
TCTACCATGGCTGGACGCGGACTACCAAGATCAAGTTTGATGACGTCGTGCAGGCCATCA
AAGACCACGCCTTTGTTACCTCGAGCTTCCCAGTGATCCTGTCCATCGAGGAGCACTGCA
GCGTGGAGCAACAGCGTCACATGGCCAAGGCCTTCAAGGAAGTATTTGGCGACCTGCTGT
TGACGAAGCCCACGGAGGCCAGTGCTGACCAGCTGCCCTCGCCCAGCCAGCTGCGGGAGA
AGATCATCATCAAGCATAAGAAGCTGGGCCCCCGAGGCGATGTGGATGTCAACATGGAGG
ACAAGAAGGACGAACACAAGCAACAGGGGGAGCTGTACATGTGGGATTCCATTGACCAGA
AATGGACTCGGCACTACTGCGCCATTGCTGATGCCAAGCTGTCCTTCAGTGATGACATTG
AACAGACTATGGAGGAGGAAGTGCCCCAGGATATACCCCCTACAGAACTACATTTTGGGG
AGAAATGGTTCCACAAGAAGGTGGAGAAGAGGACGAGTGCCGAGAAGTTGCTGCAGGAAT
ACTGCATGGAGACGGGGGGCAAGGATGGCACCTTCCTGGTTCGGGAGAGCGAGACCTTCC
CCAATGACTACACCCTGTCCTTCTGGCGGTCAGGCCGGGTCCAGCACTGCCGGATCCGCT
CCACCATGGAGGGCGGGACCCTGAAATACTACTTGACTGACAACCTGAGGTTCAGGAGGA
TGTATGCCCTCATCCAGCACTACCGCGAGACGCACCTGCCGTGCGCCGAGTTCGAGCTGC
GGCTCACGGACCCTGTGCCCAACCCCAACCCCCACGAGTCCAAGCCGTGGTACTATGACA
GCCTGAGCCGCGGAGAGGCAGAGGACATGCTGATGAGGATTCCCCGGGACGGGGCCTTCC
TGATCCGGAAGCGAGAGGGGAGCGACTCCTATGCCATCACCTTCAGGGCTAGGGGCAAGG
TAAAGCATTGTCGCATCAACCGGGACGGCCGGCACTTTGTGCTGGGGACCTCCGCCTATT
TTGAGAGTCTGGTGGAGCTCGTCAGTTACTACGAGAAGCATTCACTCTACCGAAAGATGA
GACTGCGCTACCCCGTGACCCCCGAGCTCCTGGAGCGCTACAATACGGAAAGAGATATAA
ACTCCCTCTACGACGTCAGCAGAATGTATGTGGATCCCAGTGAAATCAATCCGTCCATGC
CTCAGAGAACCGTGAAAGCTCTGTATGACTACAAAGCCAAGCGAAGCGATGAGCTGAGCT
TCTGCCGTGGTGCCCTCATCCACAATGTCTCCAAGGAGCCCGGGGGCTGGTGGAAAGGAG
ACTATGGAACCAGGATCCAGCAGTACTTCCCATCCAACTACGTCGAGGACATCTCAACTG
CAGACTTCGAGGAGCTAGAAAAGCAGATTATTGAAGACAATCCCTTAGGGTCTCTTTGCA
GAGGAATATTGGACCTCAATACCTATAACGTCGTGAAAGCCCCTCAGGGAAAAAACCAGA
AGTCCTTTGTCTTCATCCTGGAGCCCAAGGAGCAGGGCGATCCTCCGGTGGAGTTTGCCA
CAGACAGGGTGGAGGAGCTCTTTGAGTGGTTTCAGAGCATCCGAGAGATCACGTGGAAGA
TTGACAGCAAGGAGAACAACATGAAGTACTGGGAGAAGAACCAGTCCATCGCCATCGAGC
TCTCTGACCTGGTTGTCTACTGCAAACCAACCAGCAAAACCAAGGACAACTTAGAAAATC
CTGACTTCCGAGAAATCCGCTCCTTTGTGGAGACGAAGGCTGACAGCATCATCAGACAGA
AGCCCGTCGACCTCCTGAAGTACAATCAAAAGGGCCTGACCCGCGTCTACCCAAAGGGAC
AAAGAGTTGACTCTTCAAACTACGACCCCTTCCGCCTCTGGCTGTGCGGTTCTCAGATGG
TGGCACTCAATTTCCAGACGGCAGATAAGTACATGCAGATGAATCACGCATTGTTTTCTC
TCAACGGGCGCACGGGCTACGTTCTGCAGCCTGAGAGCATGAGGACAGAGAAATATGACC
CGATGCCACCCGAGTCCCAGAGGAAGATCCTGATGACGCTGACAGTCAAGGTTCTCGGTG
CTCGCCATCTCCCCAAACTTGGACGAAGTATTGCCTGTCCCTTTGTAGAAGTGGAGATCT
GTGGAGCCGAGTATGGCAACAACAAGTTCAAGACGACGGTTGTGAATGATAATGGCCTCA
GCCCTATCTGGGCTCCAACACAGGAGAAGGTGACATTTGAAATTTATGACCCAAACCTGG
CATTTCTGCGCTTTGTGGTTTATGAAGAAGATATGTTCAGCGATCCCAACTTTCTTGCTC
ATGCCACTTACCCCATTAAAGCAGTCAAATCAGGATTCAGGTCCGTTCCTCTGAAGAATG
GGTACAGCGAGGACATAGAGCTGGCTTCCCTCCTGGTTTTCTGTGAGATGCGGCCAGTCC
TGGAGAGCGAAGAGGAACTTTACTCCTCCTGTCGCCAGCTGAGGAGGCGGCAAGAAGAAC
TGAACAACCAGCTCTTTCTGTATGACACACACCAGAACTTGCGCAATGCCAACCGGGATG
CCCTGGTTAAAGAGTTCAGTGTTAATGAGAACCACTCCAGCTGTACCAGGAGAAATGCAA
CAAGAGGTTAAGAGAGAAGAGAGTCAGCAACAGCAAGTTTTACTCATAGAAGCTGGGGTA
TGTGTGTAAGGGTATTGTGTGTGTGCGCATGTGTGTTTGCATGTAGGAGAACGTGCCCTA
TTCACACTCTGGGAAGACGCTAATCTGTGACATCTTTTCTTCAAGCCTGCCATCAAGGAC
ATTTCTTAAGACCCAACTGGCATGAGTTGGGGTAATTTCCTATTATTTTCATCTTGGACA
ACTTCTAACTTATATCTTTATAGAGGATTCCCCAAAATGTGCTCCTCATTTTTGGCCTCT
CATGTTCCAAACCTCATTGAATAAAAAGCAATGAAAACCTTG

As used herein, the term “PTK2B” refers to the gene encoding Protein-tyrosine kinase 2-beta. The terms “PTK2B” and “Protein-tyrosine kinase 2-beta” include wild-type forms of the PTK2B gene, as well as variants (e.g., splice variants and polymorphisms) of wild-type PTK2B. Examples of such variants are nucleic acids having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to a wild-type PTK2B nucleic acid sequence (e.g., SEQ ID NO: 62, ENA accession number U33284). SEQ ID NO: 62 is a wild-type gene sequence encoding PTK2B protein, and is shown below:

(SEQ ID NO: 62)
CGGTACAGGTAAGTCGGCCGGGCAGGTAGGGGTGCCCGAGGAGTAGTCGCTGGAGTCCGC
GCCTCCCTGGGACTGCAATGTGCCGGTCTTAGCTGCTGCCTGAGAGGATGTCTGGGGTGT
CCGAGCCCCTGAGCCGAGTAAAGTTGGGCACATTACGCCGGCCTGAAGGCCCTGCAGAGC
CCATGGTGGTGGTACCAGTAGATGTGGAAAAGGAGGACGTGCGTATCCTCAAGGTCTGCT
TCTATAGCAACAGCTTCAATCCTGGGAAGAACTTCAAACTGGTCAAATGCACTGTCCAGA
CGGAGATCCGGGAGATCATCACCTCCATCCTGCTGAGCGGGCGGATCGGGCCCAACATCC
GGTTGGCTGAGTGCTATGGGCTGAGGCTGAAGCACATGAAGTCCGATGAGATCCACTGGC
TGCACCCACAGATGACGGTGGGTGAGGTGCAGGACAAGTATGAGTGTCTGCACGTGGAAG
CCGAGTGGAGGTATGACCTTCAAATCCGCTACTTGCCAGAAGACTTCATGGAGAGCCTGA
AGGAGGACAGGACCACGCTGCTCTATTTTTACCAACAGCTCCGGAACGACTACATGCAGC
GCTACGCCAGCAAGGTCAGCGAGGGCATGGCCCTGCAGCTGGGCTGCCTGGAGCTCAGGC
GGTTCTTCAAGGATATGCCCCACAATGCACTTGACAAGAAGTCCAACTTCGAGCTCCTAG
AAAAGGAAGTGGGGCTGGACTTGTTTTTCCCAAAGCAGATGCAGGAGAACTTAAAGCCCA
AACAGTTCCGGAAGATGATCCAGCAGACCTTCCAGCAGTACGCCTCGCTCAGGGAGGAGG
AGTGCGTCATGAAGTTCTTCAACACTCTCGCCGGCTTCGCCAACATCGACCAGGAGACCT
ACCGCTGTGAACTCATTCAAGGATGGAACATTACTGTGGACCTGGTCATTGGCCCTAAAG
GGATCCGCCAGCTGACTAGTCAGGACGCAAAGCCCACCTGCCTGGCCGAGTTCAAGCAGA
TCAGGTCCATCAGGTGCCTCCCGCTGGAGGAGGGCCAGGCAGTACTTCAGCTGGGCATTG
AAGGTGCCCCCCAGGCCTTGTCCATCAAAACCTCATCCCTAGCAGAGGCTGAGAACATGG
CTGACCTCATAGACGGCTACTGCCGGCTGCAGGGTGAGCACCAAGGCTCTCTCATCATCC
ATCCTAGGAAAGATGGTGAGAAGCGGAACAGCCTGCCCCAGATCCCCATGCTAAACCTGG
AGGCCCGGCGGTCCCACCTCTCAGAGAGCTGCAGCATAGAGTCAGACATCTACGCAGAGA
TTCCCGACGAAACCCTGCGAAGGCCCGGAGGTCCACAGTATGGCATTGCCCGTGAAGATG
TGGTCCTGAATCGTATTCTTGGGGAAGGCTTTTTTGGGGAGGTCTATGAAGGTGTCTACA
CAAATCACAAAGGGGAGAAAATCAATGTAGCTGTCAAGACCTGCAAGAAAGACTGCACTC
TGGACAACAAGGAGAAGTTCATGAGCGAGGCAGTGATCATGAAGAACCTCGACCACCCGC
ACATCGTGAAGCTGATCGGCATCATTGAAGAGGAGCCCACCTGGATCATCATGGAATTGT
ATCCCTATGGGGAGCTGGGCCACTACCTGGAGCGGAACAAGAACTCCCTGAAGGTGCTCA
CCCTCGTGCTGTACTCACTGCAGATATGCAAAGCCATGGCCTACCTGGAGAGCATCAACT
GCGTGCACAGGGACATTGCTGTCCGGAACATCCTGGTGGCCTCCCCTGAGTGTGTGAAGC
TGGGGGACTTTGGTCTTTCCCGGTACATTGAGGACGAGGACTATTACAAAGCCTCTGTGA
CTCGTCTCCCCATCAAATGGATGTCCCCAGAGTCCATTAACTTCCGACGCTTCACGACAG
CCAGTGACGTCTGGATGTTCGCCGTGTGCATGTGGGAGATCCTGAGCTTTGGGAAGCAGC
CCTTCTTCTGGCTGGAGAACAAGGATGTCATCGGGGTGCTGGAGAAAGGAGACCGGCTGC
CCAAGCCTGATCTCTGTCCACCGGTCCTTTATACCCTCATGACCCGCTGCTGGGACTACG
ACCCCAGTGACCGGCCCCGCTTCACCGAGCTGGTGTGCAGCCTCAGTGACGTTTATCAGA
TGGAGAAGGACATTGCCATGGAGCAAGAGAGGAATGCTCGCTACCGAACCCCCAAAATCT
TGGAGCCCACAGCCTTCCAGGAACCCCCACCCAAGCCCAGCCGACCTAAGTACAGACCCC
CTCCGCAAACCAACCTCCTGGCTCCAAAGCTGCAGTTCCAGGTTCCTGAGGGTCTGTGTG
CCAGCTCTCCTACGCTCACCAGCCCTATGGAGTATCCATCTCCCGTTAACTCACTGCACA
CCCCACCTCTCCACCGGCACAATGTCTTCAAACGCCACAGCATGCGGGAGGAGGACTTCA
TCCAACCCAGCAGCCGAGAAGAGGCCCAGCAGCTGTGGGAGGCTGAAAAGGTCAAAATGC
GGCAAATCCTGGACAAACAGCAGAAGCAGATGGTGGAGGACTACCAGTGGCTCAGGCAGG
AGGAGAAGTCCCTGGACCCCATGGTTTATATGAATGATAAGTCCCCATTGACGCCAGAGA
AGGAGGTCGGCTACCTGGAGTTCACAGGGCCCCCACAGAAGCCCCCGAGGCTGGGCGCAC
AGTCCATCCAGCCCACAGCTAACCTGGACCGGACCGATGACCTGGTGTACCTCAATGTCA
TGGAGCTGGTGCGGGCCGTGCTGGAGCTCAAGAATGAGCTCTGTCAGCTGCCCCCCGAGG
GCTACGTGGTGGTGGTGAAGAATGTGGGGCTGACCCTGCGGAAGCTCATCGGGAGCGTGG
ATGATCTCCTGCCTTCCTTGCCGTCATCTTCACGGACAGAGATCGAGGGCACCCAGAAAC
TGCTCAACAAAGACCTGGCAGAGCTCATCAACAAGATGCGGCTGGCGCAGCAGAACGCCG
TGACCTCCCTGAGTGAGGAGTGCAAGAGGCAGATGCTGACGGCTTCACACACCCTGGCTG
TGGACGCCAAGAACCTGCTCGACGCTGTGGACCAGGCCAAGGTTCTGGCCAATCTGGCCC
ACCCACCTGCAGAGTGACGGAGGGTGGGGGCCACCTGCCTGCGTCTTCCGCCCCTGCCTG
CCATGTACCTCCCCTGCCTTGCTGTTGGTCATGTGGGTCTTCCAGGGAGAAGGCCAAGGG
GAGTCACCTTCCCTTGCCACTTTGCACGACGCCCTCTCCCCACCCCTACCCCTGGCTGTA
CTGCTCAGGCTGCAGCTGGACAGAGGGGACTCTGGGCTATGGACACAGGGTGACGGTGAC
AAAGATGGCTCAGAGGGGGACTGCTGCTGCCTGGCCACTGCTCCCTAAGCCAGCCT

As used herein, the term “SCIMP” refers to the gene encoding SLP Adaptor and CSK Interacting Membrane Protein. The terms “SCIMP” and “SLP Adaptor and CSK Interacting Membrane Protein” include wild-type forms of the SCIMP gene, as well as variants (e.g., splice variants and polymorphisms) of wild-type SCIMP. Examples of such variants are nucleic acids having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to a wild-type SCIMP nucleic acid sequence (e.g., SEQ ID NO: 63, NCBI Reference Sequence: NM_207103.3). SEQ ID NO: 63 is a wild-type gene sequence encoding SCIMP protein, and is shown below:

(SEQ ID NO: 63)
ACTGTCTCTAGCAGTGGGTGAAGGCCTGTGAGTGAGGAATGCCTCTCACCAGCTGTGCCTGAGCTG
CAGCACTCCAGCCACTGCTGTCTCCTTAGCTGCTCACATATGGATACTTTCACAGTTCAGGATTCCAC
TGCAATGAGCTGGTGGAGGAATAATTTCTGGATCATCTTAGCTGTGGCCATCATCGTTGTCTCTGTG
GGTCTGGGCCTCATCCTGTACTGTGTCTGTAAGTGGCAGCTTAGACGAGGCAAGAAATGGGAAATT
GCCAAGCCCCTGAAACACAAGCAAGTAGATGAAGAAAAGATGTATGAGAATGTTCTTAATGAGTCGC
CAGTTCAATTACCGCCTCTGCCACCGAGGAATTGGCCTTCTCTAGAAGACTCTTCCCCACAGGAAGC
CCCAAGTCAGCCGCCCGCTACATACTCACTGGTAAATAAAGTTAAAAATAAGAAGACTGTTTCCATCC
CAAGCTACATTGAGCCTGAAGATGACTATGACGATGTTGAAATCCCTGCAAATACTGAAAAAGCATCA
TTTTGAAACAGCCATTTCTTCTTTTTGGCAAAACTGAAGAGGGTTCACACAACTTATTTTAAAACAATC
AAGAATGGTTGAACTTCAGTAGGTCTCTGGGCCCTGAAAGCCAGTGGTGATTTTATGAAGCTCTATA
AGATAAAGCACTTCCCAAACCTTAGATGAAGACACCCCTGCGATCGGATGACTGCAGCCAGAGGAG
ACACATGGGTGCTCGGCTCTGAGGACTTAGAGGGGTCAGCCTTGTGCTGTTGAGGAAACTTTCCAT
GGGAAGGACCACGGGGCTCCATGGCTCCCACCTGTGGGAAACTACTCATTTCTTGGCATTCTTTCCC
CCTTCATTCCCTTTGGTTTGCATGGTTCTGAGTGATATTAAATCTCAGCATTTGGTTGTGCAGACCCT
CCCAGGCTCCCATCCCCAGCAAGGCCCTCACCAAGCATGCTGGTCTTTACCCTCTCACCCCACCCA
CCTCCTGCACTGTGAGGCTGTGGGTGAGTTACAGCTGAGTGCTCTCGTGCCCAGGTTCCCACACCA
CATCTCGCGAGTTTGCAAGGGCAGGGAGTACCTTTTGTTCTCGTGAACCCTCCCCACCTAGACACCC
TGCAAACCCCAGTGCCTTTATATGATGTAGGCCAAATTGACCATAGAGATTTGAGTTTTCACCTAGGT
TTTCTCCCCGTGCTTGCAAGTTGTACTGTAACAATGGACAAAGGACAAAAGTTACCTTCTGATTTACA
CCTAGAAGCATCATTTTGCAATAGGTGTGTTGGGGGTGCTACAGGAAAAATACATTTCCCCCAGGAC
AAATCATGGGGAACAGGAAAGAAAAGGGGCATGTAACAATGGCATATACAAGATGAGAGTTCAGGG
GGCTTAATATCCCCTGTCCATCATTTTCATCAGTACTTACTCGAGTTCTAGGAAAACAGCCTCAAGCC
CCTTCCTTCCAGATCACTGTCCCTGGGCATCTGGGAGGAGGCAGAAGGTCCACTGTGATGTGCTGC
AGCCAATGAGATGGGCCAGGGACATGGGCAGATGTCTTGTTAAACAAGTGTCCTAATGGGGTCAAC
AAGGCCCGAGTCAGCTTTATAGGCTCTTAGACCTCATCAATTCCTTCTAGCTGATCGCCAGAGCCCT
AGGACTTGACTCATTCTAACTATACTCACAAGATGCTGGTTTCTAAGTGACCTCTGGGAAATCTGGCA
AATGAACAGCCTTGCAGAGAGAGCACTGTGAACCTGGAAAGGCCTGAGAGTGACTCAGATTTCCCT
CAAGAGATGGGAAAATGTGTTCCTCCCATTTTCAAGCTTTCTCCCTCAATCAACGCTGGAGCACTGG
GGACCTGGGCTTCCTCCCTGGTTCTCTCTTTCCAGACTCTATGAAGGCTTCCACCTTGCTATTAATAC
CTCCTTGGGAGGCCAAGGTGGGCGGATCACCTGAGGTCGGGAGTTCGAGACCAGCCTGACCAACA
TGGAGAAACCCCATTTCTACTAAAAATACAAAATTAGTCAGGCATGGTCGCGCATGCCTGTAATCCCA
GCTACTTGGGAGGCTGAGGCAGAAGAATCGCTTGAAACTGGGAGGCGGAGGTTGCGGTGAGCCGA
GAACATGCCATTGCACTCCAGCCTGGACAACAAGAGTGAAACTCCATCTAAAAATAAATAAATAAATA
AATAAATAAACCCTCCTTATGTTAGGCCAGTAGTTATCTAACTATGGCCTTATGGGACTCTGGTATCC
CACCAGCCAAAGAGAGGACTCTTCCCAAATTATAGAACAAAAATAAGCCAAAGGATTGGAGTGTTTC
AAACACATGCTTTCGTCTTATAAATGTTCTGTAAACCCTCCATGACTATGACAAAAGTTAAAAACAAAT
GCCAGACAAA

As used herein, the term “SLC24A4” refers to the gene encoding Solute Carrier Family 24 Member 4. The terms “SLC24A4” and “Solute Carrier Family 24 Member 4” include wild-type forms of the SLC24A4 gene, as well as variants (e.g., splice variants and polymorphisms) of wild-type SLC24A4. Examples of such variants are nucleic acids having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to a wild-type SLC24A4 nucleic acid sequence (e.g., SEQ ID NO: 64, NCBI Reference Sequence: NM_153646.3). SEQ ID NO: 64 is a wild-type gene sequence encoding SLC24A4 protein, and is shown below:

(SEQ ID NO: 64)
AGACGGCACCCAGGCGCTCCGGGATGGCGCTCCGCGGGACCCTCCGGCCGCTCAAAGTTCGCAG
GAGGCGAGAGATGCTGCCGCAGCAAGTCGGCTTCGTGTGCGCGGTGCTGGCCCTGGTGTGCTGTG
CGTCCGGCCTCTTCGGCAGCTTGGGGCACAAAACAGCTTCTGCTAGCAAACGTGTCCTGCCAGACA
CGTGGAGAAATAGAAAGTTGATGGCCCCAGTGAATGGGACACAGACAGCCAAGAACTGCACAGATC
CTGCGATTCACGAGTTCCCCACAGATCTGTTCTCCAATAAGGAGCGACAGCACGGAGCCGTCCTGC
TGCACATCCTTGGTGCTCTGTATATGTTCTATGCCTTGGCCATAGTGTGCGATGACTTCTTTGTTCCG
TCTCTAGAGAAGATCTGTGAGAGACTCCATCTGAGCGAAGATGTGGCTGGAGCCACCTTCATGGCT
GCAGGAAGCTCAACGCCAGAGCTGTTTGCGTCTGTTATTGGGGTGTTCATCACCCATGGGGACGTC
GGGGTGGGCACCATCGTGGGCTCTGCTGTGTTCAACATCCTGTGCATAATTGGAGTGTGCGGACTG
TTTGCTGGCCAGGTGGTCCGTCTGACGTGGTGGGCCGTGTGCCGAGACTCCGTGTACTACACCATC
TCTGTCATCGTGCTCATCGTGTTCATATATGATGAACAAATTGTGTGGTGGGAAGGCCTGGTGCTCA
TCATCTTGTATGTGTTTTATATTCTGATCATGAAGTACAATGTGAAGATGCAAGCCTTTTTCACAGTCA
AACAAAAGAGCATTGCAAACGGTAACCCGGTCAACAGTGAGCTGGAGGCTGGTAATGATTTCTATGA
CGGTAGCTATGATGACCCTTCCGTGCCATTGCTGGGGCAAGTGAAGGAGAAGCCACAGTATGGCAA
GAACCCCGTGGTGATGGTGGACGAGATTATGAGCTCCAGCCCTCCCAAGTTCACCTTCCCTGAAGC
AGGCTTACGAATCATGATCACCAATAAGTTTGGACCCAGGACCCGACTACGGATGGCCAGCAGGAT
CATCATTAATGAGCGGCAGAGACTGATCAACTCGGCCAATGGTGTGAGCAGTAAGCCGCTTCAAAAC
GGGAGGCACGAGAACATTGAGAACGGGAATGTTCCTGTGGAAAACCCCGAAGACCCTCAGCAGAAT
CAGGAGCAGCAGCCGCCGCCACAGCCACCACCGCCAGAGCCAGAGCCGGTGGAGGCTGACTTCCT
GTCCCCCTTCTCCGTGCCGGAGGCCAGAGGGGACAAGGTCAAGTGGGTGTTCACCTGGCCCCTCA
TCTTCCTCCTGTGCGTCACCATTCCCAACTGCAGCAAGCCCCGCTGGGAGAAGTTCTTCATGGTCAC
CTTCATCACCGCCACGCTGTGGATCGCTGTGTTCTCCTACATCATGGTGTGGCTGGTGACTATTATC
GGATACACACTTGGGATCCCGGATGTCATCATGGGCATTACTTTCCTGGCAGCAGGGACAAGTGTTC
CAGACTGCATGGCCAGCCTAATTGTGGCGAGACAAGGCCTTGGGGACATGGCAGTCTCCAACACCA
TAGGAAGCAACGTGTTTGACATCCTGGTAGGACTTGGTGTACCGTGGGGCCTGCAGACCATGGTTG
TTAATTATGGATCAACAGTGAAGATCAACAGCCGGGGGCTGGTCTATTCCGTGGTCCTGTTGCTGGG
CTCTGTCGCTCTCACCGTCCTCGGCATCCACCTAAACAAGTGGCGACTGGACCGGAAGCTGGGTGT
CTACGTGCTGGTTCTCTACGCCATCTTCTTGTGCTTCTCCATAATGATAGAGTTTAACGTCTTTACCTT
CGTCAACTTGCCGATGTGCCGGGAAGACGATTAGCGCTGAGTCGCGGCCCCTGGGAGCTGATCTG
GACACCCTGTGACACTGGCGTTCTCCTCTCCCCTCCTTCCCCCACCACAGGTCTCTCCTGCATAGGC
AGCCACTGTCCGTTCTTTCACACACTGGAAGGAAGAGCCATCGTGGTCTTTGTCTGGCCACAGGCCA
GGCTGCTGGGCATCCTCCTCCTCCTTGGAGTTCCGCCCCTGCAAGGCTGGATTTGGGGGCCATTAT
CTGAGCAGCTTCAAAGACCCCTGAGCTGCCAACCACGGAGATGTGCCAAGCATCTCATCTCTCCTG
CACACTTTAGTCAGAAGGACTTCTGCATGCAGTTTGTCTTTCTGTTCTGCAGGCAGCTTCAGAATTGA
GGTCATTTGTGAGCACAAGATCTCATAGGGCAGGTGCAAAATAGGAATGTTGTTCTCAAGTGTCACC
TCCAGCCCAGAGGTGGTTCCTTAGGCAGCATGTGCTCCTGGGAGCCTCTGACTTTTGCTGGAAGCA
GCCACAGTTTGGAAGGGGCAAGACCTCAACCTGTTGGGGTTTAGGGCCCATGATGGCAGACATTCT
ACCCCTTTTCCTGGAAAAACTGGAAGAATGAAAATAATTTTTTTCTGTGGAAGAGAGAAAATGAGTGA
ATATTCTTCTCACTTTTATTGATGCATTCAGAGAATAAGCAATGAAATATTAAAAAATGAAACATCATAT
AGGTCATCATACTTGAAAATTATCATTCCATATGAAAGGATCATGATACACACCAAAAAAGTAATGATC
GTAAAGACACAAATCCTCTGTATGCCATCTTGCATTGGCACTGAGGTGTTTGGTTTGGAATAGGGAA
AAAGGTAAGAGACTAACGTGGAAAGGTGCTAACTCAGAGACTGGAGATTATAGTTTACAGCTGTACT
TTCCAGATCTTCTATGTGACACAATGCACTGTCCTTGTGGGTTTGTCATTTATTGGTTAATGCTCTAGT
TTCAAAACCACCCTGTTGAAAGTTCCAGTTATTTATATGCCCAACAAATTTCATAGCCTGCTGAACTGA
ACTGAGTGTGTCAGAAGTGCTGGTTAATGACGAGAAGAGATTGCCTGAAAAACAACAAACTGCTTTC
TGGTTAGCTGAAGGCAAGTGTGAAAATCAGAATTTAGAATATTTAGAGCTAAGCTTCTGGAACCACGT
AGTTTCTACACGTGGCAGGCCAAGAATGGGAGGCTGACTCAAAACTAGATAGAAAAATATAAAATAAT
CTTCGACCACTTGATAGCTCTCAAATATATATTTAAAAGATTTATGAATACAAACCATTTATGGTTTATG
ATTTCTAAAAAGAAAGCACAATTAATTTTATAGAGAGGTTTTTTATTTTTTTAATATTTCTATTGCAAAAG
TCTATCCGATTTGATGCACTTTGAATATTGAGATATTTTGCACGGATGAATGTATGGGAACTACCCAT
GATGATGTAAGAGGAAAGAACATTTTTTTGTGATTCACCAGACATCACTTTAAACTTGGTGATGAGTTT
AAATCCAGTAGCTAATCCCTTCCTGAGACTCAAAGATCGTGACGCTGGTTGGAATTTCTGACTGTGC
CCTTTAGGGCCTCCTGAGTTTCAAAAGGAGGAAGTGTTCGTGCTTGTGTCCCTGAAGTTCCCTGTTG
CATGAGCCTGCGACAGGACCTCACCCCCACCACCAGGCTTCTATTTGGGATTCACATCAGTATTAGT
ATCGTAGCTACACCAAGTTCAGGCTTCTCTTTTTGTTTTTTTACCTAGAAATTGGGCTCAGTGGTCTTC
AACTTGAGGACGAGGGTGATTTTCCTAAGAAATCAGCAAAGAGGGAAGGCAGGGCCCCTGTAGATT
CACCAGTATAAACTTCAGCTGCAGGGATTCCAGAGCCCTCGGGACCACTCTGTCACCTTAATAGCCA
AGTTCTCCTGGTTCCTCCGATCTTACAGGCTCATCCAGGTTCCAAAGTGCTTCTGTCTCTGTTTTGAT
TCTCCAAACTGCTCTGTGATGTATGTAGGGATTATTCTCCCCACTTAACAGAAAGTAGTGTCTTGGAG
AGGTCAAGGGTCTCTAGTTCAATGGCCAGTCATAGCAGAAGGGAGGCCAAGCACCAGTCCATCACC
CCTCCCAGGCCAGCCTCTGTAAGTTGGCCACACTTGGGGAGTGAGTGTGGGTATGACTTTACCCTC
CTGGTTGGTTCTTACTGTTTGAGTCAAAACCTCATCAATATATCATTGACTCCTGGGTTCCTCAGGTC
ATTTCCTAATATCTGTCCCTATCCAATGCCTCTATTTTATCTTGAAAAAAGGACCAAAAATTATTTTTAG
CTATGGCAAGGCACAGGCCACATGGCCCCTGATGGCGTCCCTGCTGGTTTTCAATTCTCTGAAGCCT
TGTGTAGCTTTCAGAGCACACGTATCCTAATTACCCTCCTCTTCCTCAGCAGAACCCATTTGAGATTC
TAAATGAATACTCTTAGTCTCTAAAGTTGCAGTTAGAAACTAAAATAATGTTTTTTAATATGTAATATGC
TCCTCTTGGCTAATTTTCTTTTGACTTTAATGTGCCAATGTAACTTCCTTTAAAGGATCTATGCATTTAT
TAAATCTGGAAAACTATATGTACACTGTAGGTGGAAAATTCTCTTTTTTAACTAAATATTTTTCCATCAC
AAATTTAAAGAATTGCATGATTAATTAGGCTTTCATTTTTAAATTACGCTTTCATCACTACGCAGGATTA
CTTTATTTTATTCCCAAAGCTCATTAGCATGGGATAATTACTCTGCTACAGAAATAGGCAATTTAAAAA
AATGAATTTAGCTCTTCTCATTGGGGGCAGAAAAGAAAAAAAAAACCATTGCACTCAGATGGAAAATG
CCTATAGACACAGGAGCAGGTGGTTCCTGTGGACTTCTGGTTTGGAATTTTGCCTCACCAGGTCAAG
CGTGGTTAGGGTGGAAGGTGTCCAGTATCTTGAAAACCTGGCCCTGGAGGAAGGTTCTGGGTCAGC
TGCAATGAGAGACTGGTGATTAAGGGCACCGTGGGCAGGACACAGTCCTCGCCTTACCCACCCCAT
CCTTCCTGTTACCCACAGTCTGCTGGCCTCCATGCCTCTTCCCCTTGTCACTTGTGTCTCCTCCTTAT
GCACAGAGCTGCCTGCCTTTATGAATTTTCTTTTCTTTTTTTTTTGAGACAGCGTCTTGCTGTGTCACC
CAGGCTGGAGTGCAGTGGTGCCATCTTGGCTCACTGCAACCTCCGCCTCCCAGGTTCAAGCAATTC
TTGTGCCTCAGCCTCCTGAGGATTACAGGCGTGCGCCACCACACCCAGCTAATTTTTGTATTTTTAGT
AGAGACGGGTTTTCACCATGTTGGCCAGGCTGGTCTCAAACTCCTGACCTCAGGTGATCCACCCAC
CTCGGTTTCCCAAAGTGCTGGGATTACAGGTGTGAGCCACCACGCCCAGCCTGCCCTTGTGAATTTT
CACCTGCTCCTTACCCCTCACCTGTTAGGACTGTTTCTTGCTTTTGCCCCTGTCGGTCCCCTGCCTTA
ACAGACCTAAGCAGCTGATAATGCACCAAGCTTCCCTGACCAGGTGGGGTGTGTCTATCACCCAAG
GGCAGTCCTACAGACCCTGACCAAAGGCCGTTCCTGGGCGGCCCAAGGTCCAGGTTTCTTCCACCT
GCTCTTCCCTGTTTATGGGGATTTGCAAGCCTAATTGCATCAGCAGGAGCCCATCTCTCAGAGAACC
CGGACTCCCCAAGCAGACTGGGATTTTGGGAAGGGTGTGGGGGGTGTCATTGCTGGATACCCGTCT
TTCTGCCTGTCCTTTCTCCTCTCTGAATCCTGGGGCCCCTCTCCCTCCTTAAAGCTGGAGTGGACAG
AGGGACAGGAGAGGATCAGAGTTCATCCCCCCTGGGAAAGAGCAAGAGCGAATGAATCCCAGCGC
CAGCGGCTGAGGCTGCCTTCCGTGCCTTCCCTCCATGGGCGACGGGTGAGTGGGGCTTAGGAAAC
TGGAACAGGGAAGGTTCTGTTACCACACTTTGGAACTTTCCCCCTGGGATTCAGCAGTTGAGAAGCA
GAGACCTTTCTGCCCTGGGTGAATGGGTCCTTGGGGGAGGGGTTGGTCTTTTGTCTCGCATCCCCA
TCTTTCCTTTCCTTCTGGGCCATGCTCCTCCCTGGCTGGAAAAAGGTGGCTGTGCTGTCCCTGTGAT
CCACTCTCAGCAAATGCGTGTGGCTCAAATAAACAAAGAACTTACCTGTTAGAGTGAAAATCCTCAG
GAGATTGTACCCAAATGCCATGCTCTAAATATTCATGGTCTCTCTAATGCCCTCAAGACGTGATTTCC
ATGGGAACCATCCTCCCCTGGGGGCAGTTAGCAGGAGTACGTGGGGCACGTGAGGTGGTCCTCCT
TTCAGCACACCGTGCCCATAGAAACTTCTAGAAATTTCTGAAAATGCTCTGTGGGCAGCTCTTGGGT
GGCAGTAAGTCCATCAACCCCCATCTACCCCGGGCCTGAAGCGCTGCGCTTGCTCTCTTTATGTGTG
TGCACCCGAAGGATTTCCTGGTCTCTGTAGCTGATCCTGTGAGCCCCTCAAGCATGAAGCCTCCCTT
GGGGCTTCTCAAAGCATGGAGAGGGGCCCTTCCTGTCCTTTGGGAAAATCTTCCCCACTGTGTCAGT
TATATGGGAACAAGAGTGATGGGGTCTTTCTCTAGGCCTGTGCCACAGGACAGAGAACACGGGATT
CTGCTGTTCGCTTTGAGCCACAGCCTTTACCAGCCCGGCTTGTGTGGGGGGCCCCTTCGCCTTGCT
GCAAAGAGCTGTTCCCCAAAGGGCATATCCACAGGGTACAGGTTTTAAAAAGGCTTTTTTTTTTTTTT
TTGAGACAGGGTCTCGCTCTGTCGCCTAGACTCAGTGCAGTGGCGCCATGTTGGCTGGTTGCAACC
TCCACCTCCTGGGTTCAAGTGATTCTCCCACCTCAGCCTCTCTGGTAGCTGGGACTACAGGCACGC
GCCACCATGCCCAGCTAATTTTTGGATTTTTAGTAGAGAAGGAGTTTCACCATGCTGACCAGGCTGG
TTTCGAACTCCTGACCTCAAGTGATCCGCCCGCCTGGGCCTCCCAGAGTGCTGAGATTACAGGCGT
GAGCCACCGCACCTGGCCAAAAAAAGGCATTTTGATTTAGGTTGCTGTGTTTGCTTGTTGATAAAGAA
AACTCAATCGGGACACTAGTTTTGTGCTCAGCTTTAGGCCGGGTAGCTAATGGGAGGATGTCCAGCC
TGTCACTGTGCTCCCAGCGCAAGGAAATGGGTGCCCACCTGGAATCAGGAGAAGAGGCTTTTCCCT
CCTGTTCTGCAACCAGGGTGGAGCTATCTTTCCAGGGAAGCCAGCTGAGAGGTTTTAGGGCTTTGG
TTATTTTATGGGGGTTTTAAACCTCCTAACTTTTCAATGACAAATGGCTCCCAGGTGCCATAGTCTCT
GTTAAATCCTCAAACATTCACAAGCACACACTGCCAGGGGCACGGGTTGTCTTTCACCTGCATGTTT
CTAAGGCTCTTTATTCAATCTCACGGTGTCAGTGTCCAGTTGTCAAAGTTATGAATCTTCCTCCTGCT
TCTAAACAGGGCTGACAGTATACTCTCGTCTAGTCTAGGAACATGTCTGCTGCTGGGATACCCTGGT
ACCAGGATTTGAGGGCCACGGGTGGCATCTCTGAGAGCTGAAAATCCACAGAGTGCCTGTGGGAAA
GCCAAGCCCTTGGCTGTGTGGCTTTTCTATCCCTTGGATTTACAGGTCTGGGAATTGGCTGCTTCTT
AGTTATAACCCCAGTGACAAATGCTGGCTTAAGCCACACCTGTTCCCACTGTTGCTAGAATTCAAACA
GTTGCTTTTTTTTTTTCTTTTTGAGAAAGGGCCTCACTCTGTTGCCCAGGCTGGAGTGCAGTGGCTTG
ATCACAGCTCACGAAAGCCTCAAACTCCTAGGCTCAAGTGATCCTCCTGAAAAGTAGGTAGGACTAC
AGGCACATGCCACCACATACAGCTAATTTGTTTTCATTTTTTTTTTTTTTAGAGACAGGATCTCGCTGT
GTTCCCCAGGTAGGTCTTGAACTCCTGGCCTCAAGTGATCCTCCTGCCTTGACCTCCCAAAGTGCTG
GATTACAAGCGTGAGCCCCTGCACCCGGCCCAAGCAGTTGCTTCTTTTTTTCTCTTTTTTTTTTTTTTT
GAGATGGAGCCTCACTCTGTTGCCCAGGCTGGAGTGCAGTGGCGCGATCTCCACTCACTGCAAGCT
CCGCCTCCCGGGTTCATGCCATTCTCCTGCCTCAGCCTCCCGAGTAGCTGGGACTACAGGCGCCTG
CCACCACACCCAGCTAATTTTTTGTATTTTTGGTACAGACAGGGTTTCACCGTGTTAGCCAGGATGGT
CTTGATCTCCTGATCTCGTGATCCGCCCACCCCGGCCTCCCAAAGTGCTGGATTACAAGCGTGAGC
CACCGCGCCCCGCCAAGCAGTTGCTTCTTATGCAACATGTTGGTTGGGACTTGTCCACGGGCCAGG
CCAATAAAATTCTTAATCCTGCAGAGAGTCAGTACCCTCATCACCCCATCACTGGAAAACAAATGTTT
TAAGCTATCAAGAGAGGGAATGTGCAGCTTTTGGTTTCTAGATGCATGGTTTGGTGTGATCTACCTTT
GTGCCTAAAGGGAATGTCCCAAACAACAGAGCCTTCTTTGCTGTCACTCCAGAATTCTCTACACAGA
ATTTCCCAAGTCCATTCAGGACAGACGCGCAGTCCTCTTTCAATGGAAGAAGAGAGGACTTTTCCCC
TCCTGAAAAATGACTGGAGTGTGAACAAGGCAGCTCTGTTTTTCTAAATAAGTTGTTCTTGTGAGTTT
TTTCTGGCCACTGGGCATCTCTGCCCTCACTTTTCATCCCTGCCCTCTAAGCTGCAGACCCCATGAC
CACACTGTCTGCTTCCTTGAGCTTCCCGCACGAGGCTTGGACCTGGGGGACCTGGAGACCCTGCGG
ACAGAACTGTGGCTGAGCCACTGTGGCCAACTCTTGGGGAGCTCCACAGTGGGGGTTGCTGGTCTG
TGAGGCTGAGTCTCCATTTCAGAGCACACACTCCCTGGCAGGGCGCCTCTGCCTGTGTCTCCTGCC
CAGCAGCCGCCAGCAGGGAATAGTTGCTGGTGTCTGAGCACAAAGAGAGCTTTGATTACCTAGAGA
GGAAAAAGGCTGTCAGCCAGATGCAGCCAGGCCCAGGGGTAGATACAGGAGTTGCTAAGGAAGGG
GCCGAGCCAGGAGAGGCCAGGCAGATCCACAAAGCCCAAGGGGATGCAGGCTGGGTGTGGTTTCT
GAGGGAACCTACCAAATAGCAGGTAGATGGAATCAGAGGACTCTTGTGTCCTGAAAGAACCTCCTTA
AAAACAACTAAAACGAAGAACTTCTGGGGCTGTTCACACATTGTTCAAGTCACCCCAAGATCGTTCTG
GCACGCTGAGCTGAACACCACCATCTTTGTTCATTCTCTCTCTAATGGGCAAAGCAGGATCATCGAG
TTGAAAAGTTGTAAATAATGAGGATATTTATCCCGCTATTTATTTTTTCAATAACTGTGACCTCCTGCA
CTGTGAATGCTCTGTGACATGAGATTCTTAGTTTAATAAAACTGTCATTAAATTTGAATGAATTGATAT
TATTGGTTACTGAACACTGGCATGAGTTTATTTTTATTGTGAAGAAAAAAATCTACAGCAATCTAAACT
AAACCTTTCTAAGAAATCTAGCAGTCAGTATTGTAATGCAATATATCAAAATCTGTACACTGTCAATAA
AATAAATGAGCACAAAAAAAAAAAAAA

As used herein, the term “SORL1” refers to the gene encoding Sortilin-related receptor. The terms “SORL1” and “Sortilin-related receptor” include wild-type forms of the SORL1 gene, as well as variants (e.g., splice variants and polymorphisms) of wild-type SORL1. Examples of such variants are nucleic acids having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to a wild-type SORL1 nucleic acid sequence (e.g., SEQ ID NO: 65, ENA accession number Y08110). SEQ ID NO: 65 is a wild-type gene sequence encoding SORL1 protein, and is shown below:

(SEQ ID NO: 65)
CCGGCCCAGCGGCTCTCCTGGCCTCGCGCTGCACATTCTCTCCTGGCGGCGGCGCCACCT
GCAGTAGCGTTCGCCCGAACATGGCGACACGGAGCAGCAGGAGGGAGTCGCGACTCCCGT
TCCTATTCACCCTGGTCGCACTGCTGCCGCCCGGAGCTCTCTGCGAAGTCTGGACGCAGA
GGCTGCACGGCGGCAGCGCGCCCTTGCCCCAGGACCGGGGCTTCCTCGTGGTGCAGGGCG
ACCCGCGCGAGCTGCGGCTGTGGGCGCGCGGGGATGCCAGGGGGGCGAGCCGCGCGGACG
AGAAGCCGCTCCGGAGGAAACGGAGCGCTGCCCTGCAGCCCGAGCCCATCAAGGTGTACG
GACAGGTTAGTCTGAATGATTCCCACAATCAGATGGTGGTGCACTGGGCTGGAGAGAAAA
GCAACGTGATCGTGGCCTTGGCCCGAGATAGCCTGGCATTGGCGAGGCCCAAGAGCAGTG
ATGTGTACGTGTCTTACGACTATGGAAAATCATTCAAGAAAATTTCAGACAAGTTAAACT
TTGGCTTGGGAAATAGGAGTGAAGCTGTTATCGCCCAGTTCTACCACAGCCCTGCGGACA
ACAAGCGGTACATCTTTGCAGACGCTTATGCCCAGTACCTCTGGATCACGTTTGACTTCT
GCAACACTCTTCAAGGCTTTTCCATCCCATTTCGGGCAGCTGATCTCCTCCTACACAGTA
AGGCCTCCAACCTTCTCTTGGGCTTTGACAGGTCCCACCCCAACAAGCAGCTGTGGAAGT
CAGATGACTTTGGCCAGACCTGGATCATGATTCAGGAACATGTCAAGTCCTTTTCTTGGG
GAATTGATCCCTATGACAAACCAAATACCATCTACATTGAACGACACGAACCCTCTGGCT
ACTCCACTGTCTTCCGAAGTACAGATTTCTTCCAGTCCCGGGAAAACCAGGAAGTGATCC
TTGAGGAAGTGAGAGATTTTCAGCTTCGGGACAAGTACATGTTTGCTACAAAGGTGGTGC
ATCTCTTGGGCAGTGAACAGCAGTCTTCTGTCCAGCTCTGGGTCTCCTTTGGCCGGAAGC
CCATGAGAGCAGCCCAGTTTGTCACAAGACATCCTATTAATGAATATTACATCGCAGATG
CCTCCGAGGACCAGGTGTTTGTGTGTGTCAGCCACAGTAACAACCGCACCAATTTATACA
TCTCAGAGGCAGAGGGGCTGAAGTTCTCCCTGTCCTTGGAGAACGTGCTCTATTACAGCC
CAGGAGGGGCCGGCAGTGACACCTTGGTGAGGTATTTTGCAAATGAACCATTTGCTGACT
TCCACCGAGTGGAAGGATTGCAAGGAGTCTACATTGCTACTCTGATTAATGGTTCTATGA
ATGAGGAGAACATGAGATCGGTCATCACCTTTGACAAAGGGGGAACCTGGGAGTTTCTTC
AGGCTCCAGCCTTCACGGGATATGGAGAGAAAATCAATTGTGAGCTTTCCCAGGGCTGTT
CCCTTCATCTGGCTCAGCGCCTCAGTCAGCTCCTCAACCTCCAGCTCCGGAGAATGCCCA
TCCTGTCCAAGGAGTCGGCTCCAGGCCTCATCATCGCCACTGGCTCAGTGGGAAAGAACT
TGGCTAGCAAGACAAACGTGTACATCTCTAGCAGTGCTGGAGCCAGGTGGCGAGAGGCAC
TTCCTGGACCTCACTACTACACATGGGGAGACCACGGCGGAATCATCACGGCCATTGCCC
AGGGCATGGAAACCAACGAGCTAAAATACAGTACCAATGAAGGGGAGACCTGGAAAACAT
TCATCTTCTCTGAGAAGCCAGTGTTTGTGTATGGCCTCCTCACAGAACCTGGGGAGAAGA
GCACTGTCTTCACCATCTTTGGCTCGAACAAAGAGAATGTCCACAGCTGGCTGATCCTCC
AGGTCAATGCCACGGATGCCTTGGGAGTTCCCTGCACAGAGAATGACTACAAGCTGTGGT
CACCATCTGATGAGCGGGGGAATGAGTGTTTGCTGGGACACAAGACTGTTTTCAAACGGC
GGACCCCCCATGCCACATGCTTCAATGGAGAGGACTTTGACAGGCCGGTGGTCGTGTCCA
ACTGCTCCTGCACCCGGGAGGACTATGAGTGTGACTTCGGTTTCAAGATGAGTGAAGATT
TGTCATTAGAGGTTTGTGTTCCAGATCCGGAATTTTCTGGAAAGTCATACTCCCCTCCTG
TGCCTTGCCCTGTGGGTTCTACTTACAGGAGAACGAGAGGCTACCGGAAGATTTCTGGGG
ACACTTGTAGCGGAGGAGATGTTGAAGCGCGACTGGAAGGAGAGCTGGTCCCCTGTCCCC
TGGCAGAAGAGAACGAGTTCATTCTGTATGCTGTGAGGAAATCCATCTACCGCTATGACC
TGGCCTCGGGAGCCACCGAGCAGTTGCCTCTCACCGGGCTACGGGCAGCAGTGGCCCTGG
ACTTTGACTATGAGCACAACTGTTTGTATTGGTCCGACCTGGCCTTGGACGTCATCCAGC
GCCTCTGTTTGAATGGAAGCACAGGGCAAGAGGTGATCATCAATTCTGGCCTGGAGACAG
TAGAAGCTTTGGCTTTTGAACCCCTCAGCCAGCTGCTTTACTGGGTAGATGCAGGCTTCA
AAAAGATTGAGGTAGCTAATCCAGATGGCGACTTCCGACTCACAATCGTCAATTCCTCTG
TGCTTGATCGTCCCAGGGCTCTGGTCCTCGTGCCCCAAGAGGGGGTGATGTTCTGGACAG
ACTGGGGAGACCTGAAGCCTGGGATTTATCGGAGCAATATGGATGGTTCTGCTGCCTATC
ACCTGGTGTCTGAGGATGTGAAGTGGCCCAATGGCATCTCTGTGGACGACCAGTGGATTT
ACTGGACGGATGCCTACCTGGAGTGCATAGAGCGGATCACGTTCAGTGGCCAGCAGCGCT
CTGTCATTCTGGACAACCTCCCGCACCCCTATGCCATTGCTGTCTTTAAGAATGAAATCT
ACTGGGATGACTGGTCACAGCTCAGCATATTCCGAGCTTCCAAATACAGTGGGTCCCAGA
TGGAGATTCTGGCAAACCAGCTCACGGGGCTCATGGACATGAAGATTTTCTACAAGGGGA
AGAACACTGGAAGCAATGCCTGTGTGCCCAGGCCATGCAGCCTGCTGTGCCTGCCCAAGG
CCAACAACAGTAGAAGCTGCAGGTGTCCAGAGGATGTGTCCAGCAGTGTGCTTCCATCAG
GGGACCTGATGTGTGACTGCCCTCAGGGCTATCAGCTCAAGAACAATACCTGTGTCAAAG
AAGAGAACACCTGTCTTCGCAACCAGTATCGCTGCAGCAACGGGAACTGTATCAACAGCA
TTTGGTGGTGTGACTTTGACAACGACTGTGGAGACATGAGCGATGAGAGAAACTGCCCTA
CCACCATCTGTGACCTGGACACCCAGTTTCGTTGCCAGGAGTCTGGGACTTGTATCCCAC
TGTCCTATAAATGTGACCTTGAGGATGACTGTGGAGACAACAGTGATGAAAGTCATTGTG
AAATGCACCAGTGCCGGAGTGACGAGTACAACTGCAGTTCCGGCATGTGCATCCGCTCCT
CCTGGGTATGTGACGGGGACAACGACTGCAGGGACTGGTCTGATGAAGCCAACTGTACCG
CCATCTATCACACCTGTGAGGCCTCCAACTTCCAGTGCCGAAACGGGCACTGCATCCCCC
AGCGGTGGGCGTGTGACGGGGATACGGACTGCCAGGATGGTTCCGATGAGGATCCAGTCA
ACTGTGAGAAGAAGTGCAATGGATTCCGCTGCCCAAACGGCACTTGCATCCCATCCAGCA
AACATTGTGATGGTCTGCGTGATTGCTCTGATGGCTCCGATGAACAGCACTGCGAGCCCC
TCTGTACGCACTTCATGGACTTTGTGTGTAAGAACCGCCAGCAGTGCCTGTTCCACTCCA
TGGTCTGTGACGGAATCATCCAGTGCCGCGACGGGTCCGATGAGGATGCGGCGTTTGCAG
GATGCTCCCAAGATCCTGAGTTCCACAAGGTATGTGATGAGTTCGGTTTCCAGTGTCAGA
ATGGAGTGTGCATCAGTTTGATTTGGAAGTGCGACGGGATGGATGATTGCGGCGATTATT
CTGATGAAGCCAACTGCGAAAACCCCACAGAAGCCCCAAACTGCTCCCGCTACTTCCAGT
TTCGGTGTGAGAATGGCCACTGCATCCCCAACAGATGGAAATGTGACAGGGAGAACGACT
GTGGGGACTGGTCTGATGAGAAGGATTGTGGAGATTCACATATTCTTCCCTTCTCGACTC
CTGGGCCCTCCACGTGTCTGCCCAATTACTACCGCTGCAGCAGTGGGACCTGCGTGATGG
ACACCTGGGTGTGCGACGGGTACCGAGATTGTGCAGATGGCTCTGACGAGGAAGCCTGCC
CCTTGCTTGCAAACGTCACTGCTGCCTCCACTCCCACCCAACTTGGGCGATGTGACCGAT
TTGAGTTCGAATGCCACCAACCGAAGACGTGTATTCCCAACTGGAAGCGCTGTGACGGCC
ACCAAGATTGCCAGGATGGCCGGGACGAGGCCAATTGCCCCACACACAGCACCTTGACTT
GCATGAGCAGGGAGTTCCAGTGCGAGGACGGGGAGGCCTGCATTGTGCTCTCGGAGCGCT
GCGACGGCTTCCTGGACTGCTCGGACGAGAGCGATGAAAAGGCCTGCAGTGATGAGTTGA
CTGTGTACAAAGTACAGAATCTTCAGTGGACAGCTGACTTCTCTGGGGATGTGACTTTGA
CCTGGATGAGGCCCAAAAAAATGCCCTCTGCATCTTGTGTATATAATGTCTACTACAGGG
TGGTTGGAGAGAGCATATGGAAGACTCTGGAGACCCACAGCAATAAGACAAACACTGTAT
TAAAAGTCTTGAAACCAGATACCACGTATCAGGTTAAAGTACAGGTTCAGTGTCTCAGCA
AGGCACACAACACCAATGACTTTGTGACCCTGAGGACCCCAGAGGGATTGCCAGATGCCC
CTCGAAATCTCCAGCTGTCACTCCCCAGGGAAGCAGAAGGTGTGATTGTAGGCCACTGGG
CTCCTCCCATCCACACCCATGGCCTCATCCGTGAGTACATTGTAGAATACAGCAGGAGTG
GTTCCAAGATGTGGGCCTCCCAGAGGGCTGCTAGTAACTTTACAGAAATCAAGAACTTAT
TGGTCAACACTCTATACACCGTCAGAGTGGCTGCGGTGACTAGTCGTGGAATAGGAAACT
GGAGCGATTCTAAATCCATTACCACCATAAAAGGAAAAGTGATCCCACCACCAGATATCC
ACATTGACAGCTATGGTGAAAATTATCTAAGCTTCACCCTGACCATGGAGAGTGATATCA
AGGTGAATGGCTATGTGGTGAACCTTTTCTGGGCATTTGACACCCACAAGCAAGAGAGGA
GAACTTTGAACTTCCGAGGAAGCATATTGTCACACAAAGTTGGCAATCTGACAGCTCATA
CATCCTATGAGATTTCTGCCTGGGCCAAGACTGACTTGGGGGATAGCCCTCTGGCATTTG
AGCATGTTATGACCAGAGGGGTTCGCCCACCTGCACCTAGCCTCAAGGCCAAAGCCATCA
ACCAGACTGCAGTGGAATGTACCTGGACCGGCCCCCGGAATGTGGTTTATGGTATTTTCT
ATGCCACGTCCTTTCTTGACCTCTATCGCAACCCGAAGAGCTTGACTACTTCACTCCACA
ACAAGACGGTCATTGTCAGTAAGGATGAGCAGTATTTGTTTCTGGTCCGTGTAGTGGTAC
CCTACCAGGGGCCATCCTCTGACTACGTTGTAGTGAAGATGATCCCGGACAGCAGGCTTC
CACCCCGTCACCTGCATGTGGTTCATACGGGCAAAACCTCCGTGGTCATCAAGTGGGAAT
CACCGTATGACTCTCCTGACCAGGACTTGTTGTATGCAATTGCAGTCAAAGATCTCATAA
GAAAGACTGACAGGAGCTACAAAGTAAAATCCCGTAACAGCACTGTGGAATACACCCTTA
ACAAGTTGGAGCCTGGCGGGAAATACCACATCATTGTCCAACTGGGGAACATGAGCAAAG
ATTCCAGCATAAAAATTACCACAGTTTCATTATCAGCACCTGATGCCTTAAAAATCATAA
CAGAAAATGATCATGTTCTTCTGTTTTGGAAAAGCCTGGCTTTAAAGGAAAAGCATTTTA
ATGAAAGCAGGGGCTATGAGATACACATGTTTGATAGTGCCATGAATATCACAGCTTACC
TTGGGAATACTACTGACAATTTCTTTAAAATTTCCAACCTGAAGATGGGTCATAATTACA
CGTTCACCGTCCAAGCAAGATGCCTTTTTGGCAACCAGATCTGTGGGGAGCCTGCCATCC
TGCTGTACGATGAGCTGGGGTCTGGTGCAGATGCATCTGCAACGCAGGCTGCCAGATCTA
CGGATGTTGCTGCTGTGGTGGTGCCCATCTTATTCCTGATACTGCTGAGCCTGGGGGTGG
GGTTTGCCATCCTGTACACGAAGCACCGGAGGCTGCAGAGCAGCTTCACCGCCTTCGCCA
ACAGCCACTACAGCTCCAGGCTGGGGTCCGCAATCTTCTCCTCTGGGGATGACCTGGGGG
AAGATGATGAAGATGCCCCTATGATAACTGGATTTTCAGATGACGTCCCCATGGTGATAG
CCTGAAAGAGCTTTCCTCACTAGAAACCAAATGGTGTAAATATTTTATTTGATAAAGATA
GTTGATGGTTTATTTTAAAAGATGCACTTTGAGTTGCAATATGTTATTTTTATATGGGCC

As used herein, the term “SPI1” refers to the gene encoding Transcription factor PU.1. The terms “SPI1” and “Transcription factor PU.1” include wild-type forms of the SPI1 gene, as well as variants (e.g., splice variants and polymorphisms) of wild-type SPI1. Examples of such variants are nucleic acids having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to a wild-type SPI1 nucleic acid sequence (e.g., SEQ ID NO: 66, ENA accession number X52056). SEQ ID NO: 66 is a wild-type gene sequence encoding SPI1 protein, and is shown below:

(SEQ ID NO: 66)
AAAATCAGGAACTTGTGCTGGCCCTGCAATGTCAAGGGAGGGGGCTCACCCAGGGCTCCT
GTAGCTCAGGGGGCAGGCCTGAGCCCTGCACCCGCCCCACGACCGTCCAGCCCCTGACGG
GCACCCCATCCTGAGGGGCTCTGCATTGGCCCCCACCGAGGCAGGGGATCTGACCGACTC
GGAGCCCGGCTGGATGTTACAGGCGTGCAAAATGGAAGGGTTTCCCCTCGTCCCCCCTCC
ATCAGAAGACCTGGTGCCCTATGACACGGATCTATACCAACGCCAAACGCACGAGTATTA
CCCCTATCTCAGCAGTGATGGGGAGAGCCATAGCGACCATTACTGGGACTTCCACCCCCA
CCACGTGCACAGCGAGTTCGAGAGCTTCGCCGAGAACAACTTCACGGAGCTCCAGAGCGT
GCAGCCCCCGCAGCTGCAGCAGCTCTACCGCCACATGGAGCTGGAGCAGATGCACGTCCT
CGATACCCCCATGGTGCCACCCCATCCCAGTCTTGGCCACCAGGTCTCCTACCTGCCCCG
GATGTGCCTCCAGTACCCATCCCTGTCCCCAGCCCAGCCCAGCTCAGATGAGGAGGAGGG
CGAGCGGCAGAGCCCCCCACTGGAGGTGTCTGACGGCGAGGCGGATGGCCTGGAGCCCGG
GCCTGGGCTCCTGCCTGGGGAGACAGGCAGCAAGAAGAAGATCCGCCTGTACCAGTTCCT
GTTGGACCTGCTCCGCAGCGGCGACATGAAGGACAGCATCTGGTGGGTGGACAAGGACAA
GGGCACCTTCCAGTTCTCGTCCAAGCACAAGGAGGCGCTGGCGCACCGCTGGGGCATCCA
GAAGGGCAACCGCAAGAAGATGACCTACCAGAAGATGGCGCGCGCGCTGCGCAACTACGG
CAAGACGGGCGAGGTCAAGAAGGTGAAGAAGAAGCTCACCTACCAGTTCAGCGGCGAAGT
GCTGGGCCGCGGGGGCCTGGCCGAGCGGCGCCACCCGCCCCACTGAGCCCGCAGCCCCCG
CCGGCCCCGCCAGGCCTCCCCGCTGGCCATAGCATTAAGCCCTCGCCCGGCCCGGACACA
GGGAGGACGCTCCCGGGGCCCAGAGGCAGGACTGTGGCGGGCCGGGCTCCGTCACCCGCC
CCTCCCCCCACTCCAGGCCCCCTCCACATCCCGCTTCGCCTCCCTCCAGGACTCCACCCC
GGCTCCCGACGCCAGCTGGGCGTCAGACCCACCGGCAACCTTGCAGAGGACGACCCGGGG
TACTGCCTTGGGAGTCTCAAGTCCGTATGTAAATCAGATCTCCCCTCTCACCCCTCCCAC
CCATTAACCTCCTCCCAAAAAACAAGTAAAGTTATTCTCAATCC

As used herein, the term “SPP1” refers to the gene encoding Secreted Phosphoprotein 1. The terms “SPP1” and “Secreted Phosphoprotein 1” include wild-type forms of the SPP1 gene, as well as variants (e.g., splice variants and polymorphisms) of wild-type SPP1. Examples of such variants are nucleic acids having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to a wild-type SPP1 nucleic acid sequence (e.g., SEQ ID NO: 67, NCBI Reference Sequence: NM_001040058.1). SEQ ID NO: 67 is a wild-type gene sequence encoding SPP1 protein, and is shown below:

(SEQ ID NO: 67)
CTCCCTGTGTTGGTGGAGGATGTCTGCAGCAGCATTTAAATTCTGGGAGGGCTTGGTTGTCAGCAG
CAGCAGGAGGAGGCAGAGCACAGCATCGTCGGGACCAGACTCGTCTCAGGCCAGTTGCAGCCTTC
TCAGCCAAACGCCGACCAAGGAAAACTCACTACCATGAGAATTGCAGTGATTTGCTTTTGCCTCCTA
GGCATCACCTGTGCCATACCAGTTAAACAGGCTGATTCTGGAAGTTCTGAGGAAAAGCAGCTTTACA
ACAAATACCCAGATGCTGTGGCCACATGGCTAAACCCTGACCCATCTCAGAAGCAGAATCTCCTAGC
CCCACAGAATGCTGTGTCCTCTGAAGAAACCAATGACTTTAAACAAGAGACCCTTCCAAGTAAGTCC
AACGAAAGCCATGACCACATGGATGATATGGATGATGAAGATGATGATGACCATGTGGACAGCCAG
GACTCCATTGACTCGAACGACTCTGATGATGTAGATGACACTGATGATTCTCACCAGTCTGATGAGT
CTCACCATTCTGATGAATCTGATGAACTGGTCACTGATTTTCCCACGGACCTGCCAGCAACCGAAGT
TTTCACTCCAGTTGTCCCCACAGTAGACACATATGATGGCCGAGGTGATAGTGTGGTTTATGGACTG
AGGTCAAAATCTAAGAAGTTTCGCAGACCTGACATCCAGTACCCTGATGCTACAGACGAGGACATCA
CCTCACACATGGAAAGCGAGGAGTTGAATGGTGCATACAAGGCCATCCCCGTTGCCCAGGACCTGA
ACGCGCCTTCTGATTGGGACAGCCGTGGGAAGGACAGTTATGAAACGAGTCAGCTGGATGACCAGA
GTGCTGAAACCCACAGCCACAAGCAGTCCAGATTATATAAGCGGAAAGCCAATGATGAGAGCAATGA
GCATTCCGATGTGATTGATAGTCAGGAACTTTCCAAAGTCAGCCGTGAATTCCACAGCCATGAATTTC
ACAGCCATGAAGATATGCTGGTTGTAGACCCCAAAAGTAAGGAAGAAGATAAACACCTGAAATTTCG
TATTTCTCATGAATTAGATAGTGCATCTTCTGAGGTCAATTAAAAGGAGAAAAAATACAATTTCTCACT
TTGCATTTAGTCAAAAGAAAAAATGCTTTATAGCAAAATGAAAGAGAACATGAAATGCTTCTTTCTCAG
TTTATTGGTTGAATGTGTATCTATTTGAGTCTGGAAATAACTAATGTGTTTGATAATTAGTTTAGTTTGT
GGCTTCATGGAAACTCCCTGTAAACTAAAAGCTTCAGGGTTATGTCTATGTTCATTCTATAGAAGAAA
TGCAAACTATCACTGTATTTTAATATTTGTTATTCTCTCATGAATAGAAATTTATGTAGAAGCAAACAAA
ATACTTTTACCCACTTAAAAAGAGAATATAACATTTTATGTCACTATAATCTTTTGTTTTTTAAGTTAGT
GTATATTTTGTTGTGATTATCTTTTTGTGGTGTGAATAAATCTTTTATCTTGAATGTAATAAGAATTTGG
TGGTGTCAATTGCTTATTTGTTTTCCCACGGTTGTCCAGCAATTAATAAAACATAACCTTTTTTACTGC
CTAAAAAAAAAAAAAAAAA

As used herein, the term “SPPL2A” refers to the gene encoding Signal Peptide Peptidase Like 2A. The terms “SPPL2A” and “Signal Peptide Peptidase Like 2A” include wild-type forms of the SPPL2A gene, as well as variants (e.g., splice variants and polymorphisms) of wild-type SPPL2A. Examples of such variants are nucleic acids having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to a wild-type SPPL2A nucleic acid sequence (e.g., SEQ ID NO: 68, NCBI Reference Sequence: NM_001040058.1). SEQ ID NO: 68 is a wild-type gene sequence encoding SPPL2A protein, and is shown below:

(SEQ ID NO: 68)
AAGAGGAAGTCGCGCTGCTGTGGCGGCCGCTGTAGCAGCGGCGGTCCAGTCGTAGCCCGGCCGC
CCGCGCCTGTCCGGTCCGGTCCGGCCACGGAGGCAGCGCAGCGGCGGGACTCCGAGCCTACCCC
GCCGAGTGAGCTGCGCCGCACCGTGCCGTCCCACCCGGCACCCACCAGTCCGATGGGGCCGCAG
CGGCGGCTGTCCCCTGCCGGGGCCGCCCTACTCTGGGGCTTCCTGCTCCAGCTGACAGCCGCTCA
GGAAGCAATCTTGCATGCGTCTGGAAATGGCACAACCAAGGACTACTGCATGCTTTATAACCCTTATT
GGACAGCTCTTCCAAGTACCCTAGAAAATGCAACTTCCATTAGTTTGATGAATCTGACTTCCACACCA
CTATGCAACCTTTCTGATATTCCTCCTGTTGGCATAAAGAGCAAAGCAGTTGTGGTTCCATGGGGAA
GCTGCCATTTTCTTGAAAAAGCCAGAATTGCACAGAAAGGAGGTGCTGAAGCAATGTTAGTTGTCAA
TAACAGTGTCCTATTTCCTCCCTCAGGTAACAGATCTGAATTTCCTGATGTGAAAATACTGATTGCATT
TATAAGCTACAAAGACTTTAGAGATATGAACCAGACTCTAGGAGATAACATTACTGTGAAAATGTATT
CTCCATCGTGGCCTAACTTTGATTATACTATGGTGGTTATTTTTGTAATTGCGGTGTTCACTGTGGCA
TTAGGTGGATACTGGAGTGGACTAGTTGAATTGGAAAACTTGAAAGCAGTGACAACTGAAGATAGAG
AAATGAGGAAAAAGAAGGAAGAATATTTAACTTTTAGTCCTCTTACAGTTGTAATATTTGTGGTCATCT
GCTGTGTTATGATGGTCTTACTTTATTTCTTCTACAAATGGTTGGTTTATGTTATGATAGCAATTTTCTG
CATAGCATCAGCAATGAGTCTGTACAACTGTCTTGCTGCACTAATTCATAAGATACCATATGGACAAT
GCACGATTGCATGTCGTGGCAAAAACATGGAAGTGAGACTTATTTTTCTCTCTGGACTGTGCATAGC
AGTAGCTGTTGTTTGGGCTGTGTTTCGAAATGAAGACAGGTGGGCTTGGATTTTACAGGATATCTTG
GGGATTGCTTTCTGTCTGAATTTAATTAAAACACTGAAGTTGCCCAACTTCAAGTCATGTGTGATACTT
CTAGGCCTTCTCCTCCTCTATGATGTATTTTTTGTTTTCATAACACCATTCATCACAAAGAATGGTGAG
AGTATCATGGTTGAACTCGCAGCTGGACCTTTTGGAAATAATGAAAAGTTGCCAGTAGTCATCAGAGT
ACCAAAACTGATCTATTTCTCAGTAATGAGTGTGTGCCTCATGCCTGTTTCAATATTGGGTTTTGGAG
ACATTATTGTACCAGGCCTGTTGATTGCATACTGTAGAAGATTTGATGTTCAGACTGGTTCTTCTTACA
TATACTATGTTTCGTCTACAGTTGCCTATGCTATTGGCATGATACTTACATTTGTTGTTCTGGTGCTGA
TGAAAAAGGGGCAACCTGCTCTCCTCTATTTAGTACCTTGCACACTTATTACTGCCTCAGTTGTTGCC
TGGAGACGTAAGGAAATGAAAAAGTTCTGGAAAGGTAACAGCTATCAGATGATGGACCATTTGGATT
GTGCAACAAATGAAGAAAACCCTGTGATATCTGGTGAACAGATTGTCCAGCAATAATATTATGTGGAA
CTGCTATAATGTGTCATTGATTTTCTACAAATAGACTTCGACTTTTTAAATTGACTTTTGAATTGACAAT
CTGAAAGAGTCTTCAATGATATGCTTGCAAAAATATATTTTTATGAGCTGGTACTGACAGTTACATCAT
AAATAACTAAAACGCTTTGCTTTTAATGTTAAAGTTGTGCCTTCACATTAAATAAAACATATGGTCTGT
GTAGTTTCCGAGATGTACTATATACAGTATATTTTTCTAAAAAAAAA

As used herein, the term “TBK1” refers to the gene encoding Serine/threonine-protein kinase TBK1. The terms “TBK1” and “Serine/threonine-protein kinase TBK1” include wild-type forms of the TBK1 gene, as well as variants (e.g., splice variants and polymorphisms) of wild-type TBK1. Examples of such variants are nucleic acids having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to a wild-type TBK1 nucleic acid sequence (e.g., SEQ ID NO: 69, ENA accession number AF191838). SEQ ID NO: 69 is a wild-type gene sequence encoding TBK1 protein, and is shown below:

(SEQ ID NO: 69)
GCCGGCGGTGGCGCGGCGGAGACCCGGCTGGTATAACAAGAGGATTGCCTGATCCAGCCA
AGATGCAGAGCACTTCTAATCATCTGTGGCTTTTATCTGATATTTTAGGCCAAGGAGCTA
CTGCAAATGTCTTTCGTGGAAGACATAAGAAAACTGGTGATTTATTTGCTATCAAAGTAT
TTAATAACATAAGCTTCCTTCGTCCAGTGGATGTTCAAATGAGAGAATTTGAAGTGTTGA
AAAAACTCAATCACAAAAATATTGTCAAATTATTTGCTATTGAAGAGGAGACAACAACAA
GACATAAAGTACTTATTATGGAATTTTGTCCATGTGGGAGTTTATACACTGTTTTAGAAG
AACCTTCTAATGCCTATGGACTACCAGAATCTGAATTCTTAATTGTTTTGCGAGATGTGG
TGGGTGGAATGAATCATCTACGAGAGAATGGTATAGTGCACCGTGATATCAAGCCAGGAA
ATATCATGCGTGTTATAGGGGAAGATGGACAGTCTGTGTACAAACTCACAGATTTTGGTG
CAGCTAGAGAATTAGAAGATGATGAGCAGTTTGTTTCTCTGTATGGCACAGAAGAATATT
TGCACCCTGATATGTATGAGAGAGCAGTGCTAAGAAAAGATCATCAGAAGAAATATGGAG
CAACAGTTGATCTTTGGAGCATTGGGGTAACATTTTACCATGCAGCTACTGGATCACTGC
CATTTAGACCCTTTGAAGGGCCTCGTAGGAATAAAGAAGTGATGTATAAAATAATTACAG
GAAAGCCTTCTGGTGCAATATCTGGAGTACAGAAAGCAGAAAATGGACCAATTGACTGGA
GTGGAGACATGCCTGTTTCTTGCAGTCTTTCTCGGGGTCTTCAGGTTCTACTTACCCCTG
TTCTTGCAAACATCCTTGAAGCAGATCAGGAAAAGTGTTGGGGTTTTGACCAGTTTTTTG
CAGAAACTAGTGATATACTTCACCGAATGGTAATTCATGTTTTTTCGCTACAACAAATGA
CAGCTCATAAGATTTATATTCATAGCTATAATACTGCTACTATATTTCATGAACTGGTAT
ATAAACAAACCAAAATTATTTCTTCAAATCAAGAACTTATCTACGAAGGGCGACGCTTAG
TCTTAGAACCTGGAAGGCTGGCACAACATTTCCCTAAAACTACTGAGGAAAACCCTATAT
TTGTAGTAAGCCGGGAACCTCTGAATACCATAGGATTAATATATGAAAAAATTTCCCTCC
CTAAAGTACATCCACGTTATGATTTAGACGGGGATGCTAGCATGGCTAAGGCAATAACAG
GGGTTGTGTGTTATGCCTGCAGAATTGCCAGTACCTTACTGCTTTATCAGGAATTAATGC
GAAAGGGGATACGATGGCTGATTGAATTAATTAAAGATGATTACAATGAAACTGTTCACA
AAAAGACAGAAGTTGTGATCACATTGGATTTCTGTATCAGAAACATTGAAAAAACTGTGA
AAGTATATGAAAAGTTGATGAAGATCAACCTGGAAGCGGCAGAGTTAGGTGAAATTTCAG
ACATACACACCAAATTGTTGAGACTTTCCAGTTCTCAGGGAACAATAGAAACCAGTCTTC
AGGATATCGACAGCAGATTATCTCCAGGTGGATCACTGGCAGACGCATGGGCACATCAAG
AAGGCACTCATCCGAAAGACAGAAATGTAGAAAAACTACAAGTCCTGTTAAATTGCATGA
CAGAGATTTACTATCAGTTCAAAAAAGACAAAGCAGAACGTAGATTAGCTTATAATGAAG
AACAAATCCACAAATTTGATAAGCAAAAACTGTATTACCATGCCACAAAAGCTATGACGC
ACTTTACAGATGAATGTGTTAAAAAGTATGAGGCATTTTTGAATAAGTCAGAAGAATGGA
TAAGAAAGATGCTTCATCTTAGGAAACAGTTATTATCGCTGACTAATCAGTGTTTTGATA
TTGAAGAAGAAGTATCAAAATATCAAGAATATACTAATGAGTTACAAGAAACTCTGCCTC
AGAAAATGTTTACAGCTTCCAGTGGAATCAAACATACCATGACCCCAATTTATCCAAGTT
CTAACACATTAGTAGAAATGACTCTTGGTATGAAGAAATTAAAGGAAGAGATGGAAGGGG
TGGTTAAAGAACTTGCTGAAAATAACCACATTTTAGAAAGGTTTGGCTCTTTAACCATGG
ATGGTGGCCTTCGCAACGTTGACTGTCTTTAGCTTTCTAATAGAAGTTTAAGAAAAGTTT
CCGTTTGCACAAGAAAATAACGCTTGGGCATTAAATGAATGCCTTTATAGATAGTCACTT
GTTTCTACAATTCAGTATTTGATGTGGTCGTGTAAATATGTACAATATTGTAAATACATA
AAAAATATACAAATTTTTGGCTGCTGTGAAGATGTAATTTTATCTTTTAACATTTATAAT
TATATGAGGAAATTTGACCTCAGTGATCACGAGAAGAAAGCCATGACCGACCAATATGTT
GACATACTGATCCTCTACTCTGAGTGGGGCTAAATAAGTTATTTTCTCTGACCGCCTACT
GGAAATATTTTTAAGTGGAACCAAAATAGGCATCCTTACAAATCAGGAAGACTGACTTGA
CACGTTTGTAAATGGTAGAACGGTGGCTACTGTGAGTGGGGAGCAGAACCGCACCACTGT
TATACTGGGATAACAATTTTTTTGAGAAGGATAAAGTGGCATTATTTTATTTTACAAGGT
GCCCAGATCCCAGTTATCCTTGTATCCATGTAATTTCAGATGAATTATTAAGCAAACATT
TTAAAGT

As used herein, the term “TNF” refers to the gene encoding Tumor necrosis factor. The terms “TNF” and “Tumor necrosis factor” include wild-type forms of the TNF gene, as well as variants (e.g., splice variants and polymorphisms) of wild-type TNF. Examples of such variants are nucleic acids having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to a wild-type TNF nucleic acid sequence (e.g., SEQ ID NO: 70, ENA accession number X01394). SEQ ID NO: 70 is a wild-type gene sequence encoding TNF protein, and is shown below:

(SEQ ID NO: 70)
GCAGAGGACCAGCTAAGAGGGAGAGAAGCAACTACAGACCCCCCCTGAAAACAACCCTCA
GACGCCACATCCCCTGACAAGCTGCCAGGCAGGTTCTCTTCCTCTCACATACTGACCCAC
GGCTCCACCCTCTCTCCCCTGGAAAGGACACCATGAGCACTGAAAGCATGATCCGGGACG
TGGAGCTGGCCGAGGAGGCGCTCCCCAAGAAGACAGGGGGGCCCCAGGGCTCCAGGCGGT
GCTTGTTCCTCAGCCTCTTCTCCTTCCTGATCGTGGCAGGCGCCACCACGCTCTTCTGCC
TGCTGCACTTTGGAGTGATCGGCCCCCAGAGGGAAGAGTTCCCCAGGGACCTCTCTCTAA
TCAGCCCTCTGGCCCAGGCAGTCAGATCATCTTCTCGAACCCCGAGTGACAAGCCTGTAG
CCCATGTTGTAGCAAACCCTCAAGCTGAGGGGCAGCTCCAGTGGCTGAACCGCCGGGCCA
ATGCCCTCCTGGCCAATGGCGTGGAGCTGAGAGATAACCAGCTGGTGGTGCCATCAGAGG
GCCTGTACCTCATCTACTCCCAGGTCCTCTTCAAGGGCCAAGGCTGCCCCTCCACCCATG
TGCTCCTCACCCACACCATCAGCCGCATCGCCGTCTCCTACCAGACCAAGGTCAACCTCC
TCTCTGCCATCAAGAGCCCCTGCCAGAGGGAGACCCCAGAGGGGGCTGAGGCCAAGCCCT
GGTATGAGCCCATCTATCTGGGAGGGGTCTTCCAGCTGGAGAAGGGTGACCGACTCAGCG
CTGAGATCAATCGGCCCGACTATCTCGACTTTGCCGAGTCTGGGCAGGTCTACTTTGGGA
TCATTGCCCTGTGAGGAGGACGAACATCCAACCTTCCCAAACGCCTCCCCTGCCCCAATC
CCTTTATTACCCCCTCCTTCAGACACCCTCAACCTCTTCTGGCTCAAAAAGAGAATTGGG
GGCTTAGGGTCGGAACCCAAGCTTAGAACTTTAAGCAACAAGACCACCACTTCGAAACCT
GGGATTCAGGAATGTGTGGCCTGCACAGTGAATTGCTGGCAACCACTAAGAATTCAAACT
GGGGCCTCCAGAACTCACTGGGGCCTACAGCTTTGATCCCTGACATCTGGAATCTGGAGA
CCAGGGAGCCTTTGGTTCTGGCCAGAATGCTGCAGGACTTGAGAAGACCTCACCTAGAAA
TTGACACAAGTGGACCTTAGGCCTTCCTCTCTCCAGATGTTTCCAGACTTCCTTGAGACA
CGGAGCCCAGCCCTCCCCATGGAGCCAGCTCCCTCTATTTATGTTTGCACTTGTGATTAT
TTATTATTTATTTATTATTTATTTATTTACAGATGAATGTATTTATTTGGGAGACCGGGG
TATCCTGGGGGACCCAATGTAGGAGCTGCCTTGGCTCAGACATGTTTTCCGTGAAAACGG
AGCTGAACAATAGGCTGTTCCCATGTAGCCCCCTGGCCTCTGTGCCTTCTTTTGATTATG
TTTTTTAAAATATTTATCTGATTAAGTTGTCTAAACAATGCTGATTTGGTGACCAACTGT
CACTCATTGCTGAGCCTCTGCTCCCCAGGGGAGTTGTGTCTGTAATCGCCCTACTATTCA
GTGGCGAGAAATAAAGTTTGCTT

As used herein, the term “TREM2” refers to the gene encoding Triggering receptor expressed on myeloid cells 2. The terms “TREM2” and “Triggering receptor expressed on myeloid cells 2” include wild-type forms of the TREM2 gene, as well as variants (e.g., splice variants and polymorphisms) of wild-type TREM2. Examples of such variants are nucleic acids having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to a wild-type TREM2 nucleic acid sequence (e.g., SEQ ID NO: 71, ENA accession number AF213457). SEQ ID NO: 71 is a wild-type gene sequence encoding TREM2 protein, and is shown below:

(SEQ ID NO: 71)
TGACATGCCTGATCCTCTCTTTTCTGCAGTTCAAGGGAAAGACGAGATCTTGCACAAGGC
ACTCTGCTTCTGCCCTTGGCTGGGGAAGGGTGGCATGGAGCCTCTCCGGCTGCTCATCTT
ACTCTTTGTCACAGAGCTGTCCGGAGCCCACAACACCACAGTGTTCCAGGGCGTGGGGGG
CCAGTCCCTGCAGGTGTCTTGCCCCTATGACTCCATGAAGCACTGGGGGAGGCGCAAGGC
CTGGTGCCGCCAGCTGGGAGAGAAGGGCCCATGCCAGCGTGTGGTCAGCACGCACAACTT
GTGGCTGCTGTCCTTCCTGAGGAGGTGGAATGGGAGCACAGCCATCACAGACGATACCCT
GGGTGGCACTCTCACCATTACGCTGCGGAATCTACAACCCCATGATGCGGGTCTCTACCA
GTGCCAGAGCCTCCATGGCAGTGAGGCTGACACCCTCAGGAAGGTCCTGGTGGAGGTGCT
GGCAGACCCCCTGGATCACCGGGATGCTGGAGATCTCTGGTTCCCCGGGGAGTCTGAGAG
CTTCGAGGATGCCCATGTGGAGCACAGCATCTCCAGGAGCCTCTTGGAAGGAGAAATCCC
CTTCCCACCCACTTCCATCCTTCTCCTCCTGGCCTGCATCTTTCTCATCAAGATTCTAGC
AGCCAGCGCCCTCTGGGCTGCAGCCTGGCATGGACAGAAGCCAGGGACACATCCACCCAG
TGAACTGGACTGTGGCCATGACCCAGGGTATCAGCTCCAAACTCTGCCAGGGCTGAGAGA
CACGTGAAGGAAGATGATGGGAGGAAAAGCCCAGGAGAAGTCCCACCAGGGACCAGCCCA
GCCTGCATACTTGCCACTTGGCCACCAGGACTCCTTGTTCTGCTCTGGCAAGAGACTACT
CTGCCTGAACACTGCTTCTCCTGGACCCTGGAAGCAGGGACTGGTTGAGGGAGTGGGGAG
GTGGTAAGAACACCTGACAACTTCTGAATATTGGACATTTTAAACACTTACAAATAAATC
CAAGACTGTCATATTTAAAAA

As used herein, the term “TREML2” refers to the gene encoding Triggering Receptor Expressed on Myeloid Cells Like 2. The terms “TREML2” and “Triggering Receptor Expressed on Myeloid Cells Like 2” include wild-type forms of the TREML2 gene, as well as variants (e.g., splice variants and polymorphisms) of wild-type TREML2. Examples of such variants are nucleic acids having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to a wild-type TREML2 nucleic acid sequence (e.g., SEQ ID NO: 72, NCBI Reference Sequence: NM_024807.3). SEQ ID NO: 72 is a wild-type gene sequence encoding TREML2 protein, and is shown below:

(SEQ ID NO: 72)
CAATGAATCCCTGCGGTTGGCTGGGGGCAGTGGGTCCCACACTGCCTCACTTCCCTAAATGGGCAG
CTTCACTTTTAGAACCCCGGGTCCTTCCCTGGCAGGCCCAGGTGGCACATCCTGTGTCGGGTGGGC
CCTCACCTTGGATCTCCAGGCCTGACACTGCCCAGCTGGATGGAACCATGGCCCCAGCCTTCCTGC
TGCTGCTGCTGCTGTGGCCACAGGGTTGCGTCTCAGGCCCCTCTGCTGACAGTGTATACACAAAAG
TGAGGCTCCTTGAAGGGGAGACTCTGTCTGTGCAGTGCTCCTATAAGGGCTACAAAAACCGCGTGG
AGGGCAAGGTTTGGTGCAAAATCAGGAAGAAGAAGTGTGAGCCTGGCTTTGCCCGAGTCTGGGTGA
AAGGGCCCCGCTACTTGCTGCAGGACGATGCCCAGGCCAAGGTGGTCAACATCACCATGGTGGCC
CTCAAGCTCCAGGACTCAGGCCGATACTGGTGCATGCGCAACACCTCTGGGATCCTGTACCCCTTG
ATGGGCTTCCAGCTGGATGTGTCTCCAGCTCCCCAAACTGAGAGGAACATTCCTTTCACACATCTGG
ACAACATCCTCAAGAGTGGAACTGTCACAACTGGCCAAGCCCCTACCTCAGGCCCTGATGCCCCTTT
TACCACTGGTGTGATGGTGTTCACCCCAGGACTCATCACCTTGCCTAGGCTCTTAGCCTCCACCAGA
CCTGCCTCCAAGACAGGCTACAGCTTCACTGCTACCAGCACCACCAGCCAGGGACCCAGGAGGACC
ATGGGGTCCCAGACAGTGACCGCGTCTCCCAGCAATGCCAGGGACTCCTCTGCTGGCCCAGAATCC
ATCTCCACTAAGTCTGGGGACCTCAGCACCAGATCGCCCACCACAGGGCTCTGCCTCACCAGCAGA
TCTCTCCTCAACAGACTACCCTCCATGCCCTCCATCAGGCACCAGGATGTTTACTCCACTGTGCTTG
GGGTGGTGCTGACCCTCCTGGTGCTGATGCTGATCATGGTCTATGGGTTTTGGAAGAAGAGACACA
TGGCAAGCTACAGCATGTGCAGCGATCCTTCTACACGTGACCCACCTGGAAGACCAGAGCCCTATG
TGGAAGTCTACTTGATCTGAGGCCACTTAAGCATGGGGTGGGGAGCTTCTCCCAGAGTGGCCCCAG
GGGGTTAGAGGAGGGGTGAAGATTGGGGCCAGTATCGATCTTATGAAGCTGGAGGACTTGTGCAGT
GCTGGACTCACCCAGGACTTCCCAAACCCAGAGGCTGCCATCCTAAGCAGCCCCACAGCCCAGTGT
TCTCCTTGGGGGCAGGAACCTGGGGAGGGGCCCAGAGCAAAGGGCATCAGGGAGAAAGTCCCGAG
GAAATGTGACCAGTGGTTTCTGCTCGGAGCTGCAGACCCCAGGGCTCTTGGTGGAGGCAGGGGAA
CCCTGAGAGTGCTGTTTACAGAGAACCTCAGCTCCCGTCTGCCTCAGAAACCCTATTGGGCTGAGCT
GCCCTCCCCACCAGGGCCACTGTGTCCTCTGCTTCCCTCCGTTCTGCTTCAGCTTCCCCTAAGGTTA
GGGAAGAAAGAATCGGGCTCACGAATGCCAGAGGCAGTGATGTCCCATCCTGGAGGAGAGGAAAC
AGTGACTAAAAGCTGGGGACCCACAGAGGGGTTGGCAGCTTCTCTTGTCGGGACAGGTGTCCTTTG
CTGGGCCTCTGGATGGCCCTGCCCTGACTGGGGCTGCTCCTCCCTCCTGTCCTGGGACCGCGCAG
AGCCCACGCTCTCACTGCTGCCTCCTGCTGGCCGCTGCCTCCTTAGAAAGCTGTGACCAGGCAGCT
AAGAGCCTCTGGGCTGCAGGGTCAGCCTCTCCCAAGACTGAAGTGCAGAGGCTGGACTTGGGGCT
CTCTCCCCCAGCTTCTACACCTGGGCTCCAAGTCTGAGTTCCCACAGGGGACCCAGCAGCCTCCAG
GAAGTCCATACCCTGGGGTGGCTGAGACCTTGGCTCTGTATGGAGGCTGCTCACCCCACAGACACT
GGTGGGGAGACCATGGCTCAGAGGAAGGGTGGAGCAACCCTCCTCCTACCCCTCAGGATAGAGAG
AGAAGACACACTTGGGACACAGTGAAGACAGTAACTTGGAACTGACCACGGCCTGGAGGACTGGCC
CAGGCAGGGGGACAGGGAAAATGGAGCCCAAGTAGCCTCTGGCCAGGGACCCAATGTCCCGAGGA
ATCTGCCTCCCACCCACTGACTCAGGGCTCAGACTCAGCCTCTATTGTCCAGAGCACTGGCTTGGC
GTCCAGCAATGAAGGCTGGAGAATGCAGCCTGGATTCCCCTACACACACACACACACACACACACA
CACACACACACACACACACACACACACAGGTGTCTACTGACCTGGAGTGACTGGAATAGCACCTGG
GGATAAATGTGACAACTGTGCATTGAACCCTGGGTCAGGGACGTTCCAATGGCCAAGAGAGTGACA
CAGCCAGGACCCTGGTGGACAGCCAGAGGGGCCACTTCAGGATGGATGTGGGGAGAGTGGAAGAG
GCAGGGAGTAATCCTGGGGGACAGCAGGGAGGAGGCACTTCTTCCCTATGTCCAGGAGAGGGCAA
TAGAGGGAAGACTGAGGCTGAAGAATTGACGGCTCTGGACCCAGGACAGACAGACAGACAGACAGA
CAGACAGACAGACAGACACGCACACACACCCATCTCTGTCTAGCAAGCAGCCTCCTAAGATAGCTGT
TCTCCCTATCATGACGGTGTAGCCACCATCCTGTTGTATACTAGGAGAGAACTTAACCCACCTGGGG
GAAAATAGCTCCCCAAGAGCTGGCACCAGTACCACTGATGGCCCTGCTTCCTCTGAGTGAGATGCC
CAGGAGGAGGAGCCCTAGGGAAGAAGTCAGGGACAGGGACCAGGATACCACTCTGTCACTGTGTG
ACCCTCAGCAAGTCACTAACCCTTGGCCTCATTTTTCCTGTCTTGTGAAAGAGGACAATAATTCCTAC
TTCTCAAGATTGTTTTCAAGATAAAATAACATTAGCATTGTACAATGATGCAAATGCCTCATTACCATT
ATTCCTTAAGTTGTTTTCCAGCTCTAATGTTGTTTCCAACATTACATTTAAGACCTTAGGATTCTGTTTC
TTGCTTTTGTCATATCTCTTCCCAAGTGTCATCACTATATGGATGTTGAGGGCCCCCGATGACAGTCC
CTTTGGTAAGGTCCTCTTTTGAGGAGGGGAGGGTACAGGGTGGACTCATCTCAGTGTGAACTTGGC
AAGTCACTGTCCCTCTCTGATCTTGTTTCCTCATCTGGAGAAGGAGTGAGAGAGGAGAAAGGAAGAA
ACCAGTCAGGCAGGCAGTTAGGGTGGGTTCTCGGTAGAATTCTTTTAAACAAAAGAACAGCCTGAAA
AATCAAGCTGCAGGCACAGATATGGGAACTTGCACAGGGGGGCTTGCCTAAGACATGCCCACAGCC
TCATAGATAAGACAGACTACACAGGTGACTTGCCCAAACATGCCTGCAATGGAAAATTTCATCCCCT
GACATGTGCAGTAAGGGGAACAAAGCAATATGGAGTAAGTAACTCAAGCCAAGGGCCCACATGTAC
ATTAGAAGGACAGCAGGGAGCTACCAGAAATTCATGCCTTATGCAGATGAGCTGCCCAGTCCTCATC
GGTTTCTTATAAAAGCCTTTACATTCAACTGTAAAAATGGCAACCCTCTTTCAGGCCTCCTCTCCACA
GCAGAGAGCTTTCTTCTCTCACTCATTAAACTTTCACTCCAACCTCAAAAAAAAAAAAAAAAAA

As used herein, the term “TYROBP” refers to the gene encoding TYRO protein tyrosine kinase-binding protein. The terms “TYROBP” and “TYRO protein tyrosine kinase-binding protein” include wild-type forms of the TYROBP gene, as well as variants (e.g., splice variants and polymorphisms) of wild-type TYROBP. Examples of such variants are nucleic acids having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to a wild-type TYROBP nucleic acid sequence (e.g., SEQ ID NO: 73, ENA accession number AF019562). SEQ ID NO: 73 is a wild-type gene sequence encoding TYROBP protein, and is shown below:

(SEQ ID NO: 73)
CCACGCGTCCGCGCTGCGCCACATCCCACCGGCCCTTACACTGTGGTGTCCAGCAGCATC
CGGCTTCATGGGGGGACTTGAACCCTGCAGCAGGCTCCTGCTCCTGCCTCTCCTGCTGGC
TGTAAGTGGTCTCCGTCCTGTCCAGGCCCAGGCCCAGAGCGATTGCAGTTGCTCTACGGT
GAGCCCGGGCGTGCTGGCAGGGATCGTGATGGGAGACCTGGTGCTGACAGTGCTCATTGC
CCTGGCCGTGTACTTCCTGGGCCGGCTGGTCCCTCGGGGGCGAGGGGCTGCGGAGGCAGC
GACCCGGAAACAGCGTATCACTGAGACCGAGTCGCCTTATCAGGAGCTCCAGGGTCAGAG
GTCGGATGTCTACAGCGACCTCAACACACAGAGGCCGTATTACAAATGAGCCCGAATCAT
GACAGTCAGCAACATGATACCTGGATCCAGCCATTCCTGAAGCCCACCCTGCACCTCATT
CCAACTCCTACCGCGATACAGACCCACAGAGTGCCATCCCTGAGAGACCAGACCGCTCCC
CAATACTCTCCTAAAATAAACATGAAGCACAAAAAAAAAAAAAAAAAAAAAAAAAAAAAAAAAA

As used herein, the term “ZCWPW1” refers to the gene encoding Zinc finger CW-type PWWP domain protein 1. The terms “ZCWPW1” and “Zinc finger CW-type PWWP domain protein 1” include wild-type forms of the ZCWPW1 gene, as well as variants (e.g., splice variants and polymorphisms) of wild-type ZCWPW1. Examples of such variants are nucleic acids having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to a wild-type ZCWPW1 nucleic acid sequence (e.g., SEQ ID NO: 74, ENA accession number AL136735). SEQ ID NO: 74 is a wild-type gene sequence encoding ZCWPW1 protein, and is shown below:

(SEQ ID NO: 74)
CGCCGTTTTCCCGGGGAGATGCGCCGCCCGGTCTCCCTGCCAGCGGAGTGCTGGGCCGAG
GACAGGGCGGCAGGGGTGACAGTGGGGTCCAGGAGAGTCTCAAAATCCTAAGCTTTCAGT
ATTTGTTATTGTGAAAGAAGTTAATTCACCTGAAACAGAGGAGGGGCAACCTGAGTTATC
AGAAAGTGACTTCCTGGCCTTCCCTTCTTTACTGATCAGAGGCACACAAAGCGTAGTTTC
TAAGCTGAATGATGACAACGTTGCAGAATAAAGAAGAATGTGGAAAGGGACCAAAGAGAA
TCTTTGCCCCACCTGCACAAAAATCTTACAGCCTGTTACCTTGTAGCCCTAACTCCCCTA
AGGAGGAGACCCCGGGGATCAGTTCCCCAGAGACAGAGGCCAGGATAAGCCTGCCAAAGG
CCAGTTTAAAGAAGAAAGAGGAAAAAGCAACCATGAAGAATGTTCCAAGCAGGGAACAGG
AGAAAAAAAGAAAGGCACAAATCAACAAGCAAGCAGAGAAGAAAGAAAAGGAAAAATCAA
GTCTTACCAATGCAGAATTTGAGGAGATTGTCCAGATTGTTCTGCAGAAGTCCCTTCAGG
AGTGCTTGGGGATGGGATCTGGCCTTGATTTTGCAGAGACTTCTTGTGCCCAGCCCGTAG
TATCTACCCAATCAGACAAGGAGCCAGGAATTACTGCTTCTGCTACTGATACTGATAATG
CTAATGGAGAGGAGGTACCACATACTCAAGAGATTTCAGTGTCTTGGGAAGGTGAAGCTG
CCCCTGAGATAAGGACATCTAAGTTAGGCCAGCCAGATCCTGCACCCTCTAAGAAGAAAT
CCAATAGACTCACCTTAAGCAAAAGAAAGAAGGAAGCTCATGAGAAGGTGGAGAAAACTC
AAGGTGGACATGAGCACAGACAGGAAGACCGACTAAAGAAAACAGTTCAGGATCATTCTC
AGATCAGGGACCAGCAAAAAGGAGAGATAAGTGGTTTTGGTCAATGTCTGGTCTGGGTCC
AGTGTTCCTTCCCAAACTGTGGGAAATGGAGGCGGCTGTGTGGGAACATTGACCCCTCAG
TTCTCCCAGATAATTGGTCCTGTGATCAGAACACAGATGTGCAGTATAATCGCTGTGATA
TTCCTGAGGAGACCTGGACAGGGCTTGAGAGTGATGTGGCCTATGCCTCCTACATCCCAG
GATCCATCATCTGGGCCAAGCAATACGGTTACCCCTGGTGGCCAGGCATGATAGAATCTG
ATCCTGACTTAGGGGAATATTTTCTTTTTACTTCCCATCTTGATTCCCTGCCGTCTAAGT
ACCATGTGACGTTTTTTGGAGAAACAGTTTCTCGTGCATGGATCCCAGTCAACATGCTAA
AGAACTTCCAGGAGCTGTCCCTGGAGCTATCAGTCATGAAAAAGCGCAGAAATGACTGCA
GCCAGAAACTGGGGGTGGCCCTGATGATGGCTCAAGAGGCAGAACAGATCAGCATTCAGG
AACGGGTTAACTTGTTTGGTTTCTGGAGCCGATTCAACGGATCTAACAGTAATGGGGAAA
GAAAAGACTTACAGCTCTCTGGTTTGAACAGCCCAGGATCCTGOTTAGAGAAAAAGGAGA
AAGAGGAAGAGTTGGAAAAGGAGGAAGGAGAGAAAACAGACCCAATTTTGCCCATTCGTA
AGCGAGTCAAAATACAGACCCAAAAAAACCAAGCCAAGAGGGCTTGGGGGTGATGCAGGC
ACAGCAGATGGCCGAGGCAGGACACTGCAGAGGAAGATAATGAAGAGATCTCTAGGCAGG
AAATCCACAGCTCCTCCTGCACCCAGAATGGGAAGGAAAGAAGGCCAAGGGAATTCAGAT
TCTGACCAGCCAGGCCCTAAGAAAAAATTTAAAGCTCCCCAGAGCAAGGCCTTGGCAGCC
AGCTTTTCAGAGGGAAAAGAAGTTAGAACAGTGCCAAAGAACCTGGGCCTATCAGCGTGT
AAGGGGGCCTGCCCCTCATCTGCGAAAGAAGAGCCCAGACACCGGGAACCCCTGACCCAG
GAGGCTGGAAGTGTCCCCCTTGAGGACGAAGCCTCCAGTGACCTGGACCTGGAGCAACTC
ATGGAAGATGTTGGGAGAGAGCTGGGGCAGAGCGGGGAGCTGCAGCACAGCAACAGTGAT
GGCGAGGACTTCCCCGTGGCGCTGTTTGGGAAGTAGCTGGTGCTCCTCTGCTCCCTCTTT
TTCTCCCTTCTCTGGGGCGCAGGAGGGAGAAGTTGCTAAGTGCTGGGTCTGTTCATTGGC
TATGAGGTTCAAATGTGTGTGGTGCAGTTTCTGTGTTAATAAAGCAGGTTACAGTCGAAA
AAAAAAAAAAAAAAAAA

### DETAILED DESCRIPTION OF THE INVENTION

The present invention provides new forms of siRNA, including single- and double-stranded short interfering RNA (ds-siRNA), and methods for their use in treating a patient in need of microglial gene silencing (e.g., a patient having dysregulated microglial gene expression, such as a patient with, e.g., Alzheimer's disease, amyotrophic lateral sclerosis, Parkinson's disease, frontotemporal dementia, Huntington's disease, multiple sclerosis, or progressive supranuclear palsy). The branched siRNA in the present invention has shown a surprising ability to permeate the cell. The branched compositions described herein may employ a variety of modifications known and previously unknown in the art. The siRNA of the invention may contain an antisense strand including a region that is represented by Formula IX:

Z-((A-P-)ₙ(B-P-)ₘ)_{q}; (IX)

wherein Z is a 5′ phosphorus stabilizing moiety; each A is, independently, a 2′-modified-ribonucleoside of a first type; each B is, independently, a 2′-modified-ribonucleoside of a second type; each P is, independently, an internucleoside linkage selected from a phosphodiester linkage and a phosphorothioate linkage; n is an integer from 1 to 5; m is an integer from 1 to 5; and q is an integer between 1 and 15. The embodiments of each part of Formula IX and the methods of use for the molecules Formula IX represents are described herein.

In some embodiments, the siRNA of the invention may have a sense strand represented by Formula X:

Y-((A-P-)ₙ(B-P-)ₘ)_{q}L-((B-P-)ₘ(A-P-)ₙ)_{q}; (X)

wherein Y is a hydrophobic moiety (e.g., cholesterol, vitamin D, or tocopherol); Lisa linker; each A is, independently, a 2′-modified-ribonucleoside of a first type; each B is, independently, a 2′-modified-ribonucleoside of a second type; each P is, independently, an internucleoside linkage selected from a phosphodiester linkage and a phosphorothioate linkage; n is an integer from 1 to 5; m is an integer from 1 to 5; and q is an integer between 1 and 15. The embodiments of each part of Formula X and the methods of use for the molecules Formula X represents are described herein.
siRNA Structure

The simplest siRNAs consist of a ribonucleic acid comprising a single- or double-stranded structure, formed by a first strand, and in the case of a double-stranded siRNA, a second strand. The first strand comprises a stretch of contiguous nucleotides that is at least partially complementary to a target nucleic acid. The second strand also comprises a stretch of contiguous nucleotides where the second stretch is at least partially identical to a target nucleic acid. The first strand and said second strand may be hybridized to each other to form a double-stranded structure. The hybridization typically occurs by Watson Crick base pairing.

Depending on the sequence of the first and second strand, the hybridization or base pairing is not necessarily complete or perfect, which means that the first and second strand are not 100% base-paired due to mismatches. One or more mismatches may also be present within the duplex without necessarily impacting the siRNA activity.

The first strand contains a stretch of contiguous nucleotides which is essentially complementary to a target nucleic acid. Typically, the target nucleic acid sequence is, in accordance with the mode of action of interfering ribonucleic acids, a single-stranded RNA, preferably an mRNA. Such hybridization occurs most likely through Watson Crick base pairing but is not necessarily limited thereto. The extent to which the first strand has a complementary stretch of contiguous nucleotides to a target nucleic acid sequence can be between 80% and 100%, e.g., 80%, 85%, 90%, 95%, or 100% complementary.

siRNAs described herein may employ modifications to the nucleobase, phosphate backbone, ribose core, 5′- and 3′-ends, and branching, wherein multiple strands of siRNA may be covalently linked.

Length of siRNA Molecules

It is within the scope of the invention that any length, known and previously unknown in the art, may be employed for the current invention. As described herein, potential lengths for an antisense strand of the branched siRNA of the present invention is between 10 and 30 nucleotides (e.g., 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, or 30 nucleotides), 15 and 25 nucleotides (e.g., 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, or 25 nucleotides), or 18 and 23 nucleotides (e.g., 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, or 30 nucleotides). In some embodiments, the antisense strand is 21 nucleotides. In some embodiments, the antisense strand is 22 nucleotides. In some embodiments, the antisense strand is 23 nucleotides. In some embodiments, the antisense strand is 24 nucleotides. In some embodiments, the antisense strand is 25 nucleotides. In some embodiments, the antisense strand is 26 nucleotides. In some embodiments, the antisense strand is 27 nucleotides. In some embodiments, the antisense strand is 28 nucleotides. In some embodiments, the antisense strand is 29 nucleotides. In some embodiments, the antisense strand is 30 nucleotides.

In some embodiments, the sense strand of the branched siRNA of the present invention is between 12 and 30 nucleotides (e.g., 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, or 30 nucleotides), or 14 and 18 nucleotides (e.g., 14 nucleotides, 15 nucleotides, 16 nucleotides, 17 nucleotides, or 18 nucleotides). In some embodiments, the sense strand is 16 nucleotides. In some embodiments, the sense strand is 17 nucleotides. In some embodiments, the sense strand is 18 nucleotides. In some embodiments, the sense strand is 19 nucleotides. In some embodiments, the sense strand is 20 nucleotides. In some embodiments, the sense strand is 21 nucleotides. In some embodiments, the sense strand is 22 nucleotides. In some embodiments, the sense strand is 23 nucleotides. In some embodiments, the sense strand is 24 nucleotides. In some embodiments, the sense strand is 25 nucleotides. In some embodiments, the sense strand is 26 nucleotides. In some embodiments, the sense strand is 27 nucleotides. In some embodiments, the sense strand is 28 nucleotides. In some embodiments, the sense strand is 29 nucleotides. In some embodiments, the sense strand is 30 nucleotides.

2′ Modifications

The present invention includes single- and double-stranded compositions comprising at least one alternating motif. Alternating motifs of the present invention may have the formula ((A-P-)ₙ(B-P-)ₘ) q where A is a nucleoside of a first type, B is a nucleoside of a second type, n is from 1 to 5, m is from 1 to 5, and q is from 1 to 15, and P is an internucleoside linkage. The result may include a regular or irregular pattern of alternating nucleosides of the first and second types. Each of the types of nucleosides may be identical with the exception that at least the 2′-substituent groups are different.

Possible 2′-modifications comprise all possible orientations of OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C1 to C10 alkyl or C2 to C10 alkenyl and alkynyl. In some embodiments, the modification includes a 2′-O-methyl (2′-O-Me) modification. Some embodiments use O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃]₂, where n and m are from 1 to about 10. Other potential sugar substituent groups include: C1 to C10 lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, a group for improving the pharmacokinetic properties of an oligonucleotide, or a group for improving the pharmacodynamic properties of an oligonucleotide, and other substituents having similar properties. In some embodiments, the modification includes 2′ methoxyethoxy (2′-O—CH₂CH₂OCH₃, also known as 2′-O-(2-methoxyethyl) or 2′-MOE). In some embodiments, the modification includes 2′-dimethylaminooxyethoxy, i.e., a O(CH₂)₂ON(CH₃)₂ group, also known as 2′-DMAOE, and 2′-dimethylaminoethoxyethoxy (also known in the art as 2′-O-dimethylamino-ethoxy-ethyl or 2′-DMAEOE), i.e., 2′-O—CH₂OCH₂N(CH₃)₂. Other potential sugar substituent groups include aminopropoxy (—OCH₂CH₂CH₂NH₂), allyl (—CH₂—CH═CH₂), —O-allyl (—O—CH₂—CH═CH₂) and fluoro (F). 2′-sugar substituent groups may be in the arabino (up) position or ribo (down) position. In some embodiments, the 2′-arabino modification is 2′-F. Similar modifications may also be made at other positions on the oligomeric compound, particularly the 3′ position of the sugar on the 3′ terminal nucleoside or in 2′-5′ linked oligonucleotides and the 5′ position of 5′ terminal nucleotide. Oligonucleotides may also have sugar mimetics such as cyclobutyl moieties in place of the pentofuranosyl sugar.

Nucleobase Modifications

Oligomeric compounds may also include nucleosides or other surrogate or mimetic monomeric subunits that include a nucleobase (often referred to in the art simply as “base” or “heterocyclic base moiety”). The nucleobase is another moiety that has been extensively modified or substituted and such modified and or substituted nucleobases are amenable to the present invention. As used herein, “unmodified” or “natural” nucleobases include the purine bases adenine (A) and guanine (G), and the pyrimidine bases thymine (T), cytosine (C) and uracil (U). Modified nucleobases also referred herein as heterocyclic base moieties include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C═C-CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in The Concise Encyclopedia of Polymer Science and Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and, Applications, pages 289-302, Crooke, S. T. and Lebleu, B., ed., CRC Press, 1993. Oligomeric compounds of the present invention can also include polycyclic heterocyclic compounds in place of one or more heterocyclic base moieties. A number of tricyclic heterocyclic compounds have been previously reported. These compounds are routinely used in antisense applications to increase the binding properties of the modified strand to a target strand.

Representative cytosine analogs that make 3 hydrogen bonds with a guanosine in a second strand include 1,3-diazaphenoxazine-2-one (Kurchavov, et al., Nucleosides and Nucleotides, 1997, 16, 1837-1846), 1,3-diazaphenothiazine-2-one (Lin, K.-Y.; Jones, R. J.; Matteucci, M. J. Am. Chem. Soc. 1995, 117, 3873-3874), and 6,7,8,9-tetrafluoro-1,3-diazaphenoxazine-2-one (Wang, J.; Lin, K.-Y., Matteucci, M. Tetrahedron Lett. 1998, 39, 8385-8388). Incorporated into oligonucleotides these base modifications were shown to hybridize with complementary guanine and the latter was also shown to hybridize with adenine and to enhance helical thermal stability by extended stacking interactions (also see U.S. patent application entitled “Modified Peptide Nucleic Acids” filed May 24, 2002, Ser. No. 10/155,920; and U.S. patent application entitled “Nuclease Resistant Chimeric Oligonucleotides” filed May 24, 2002, Ser. No. 10/013,295, both of which are herein incorporated by reference in their entirety). Further helix-stabilizing properties have been observed when a cytosine analog/substitute has an aminoethoxy moiety attached to the rigid 1,3-diazaphenoxazine-2-one scaffold (Lin, K.-Y.; Matteucci, M. J. 25 Am. Chem. Soc. 1998, 120, 8531-8532).

Internucleoside Linkage Modifications

Another variable in the design of the present invention are the internucleoside linkages making up the phosphate backbone. Although the natural RNA phosphate backbone may be employed here, derivatives thereof, known and yet unknown in the art, may be used which enhance desirable characteristics of a siRNA. Although not limiting, of particular importance in the present invention is protecting parts, or the whole, of the siRNA from hydrolysis. One example of a modification that decreases the rate of hydrolysis is phosphorothioates. Any portion or the whole of the backbone may contain phosphate substitutions (e.g., phosphorothioates, phosphodiesters, etc.). For instance, the internucleoside linkages may be between 0 and 100% phosphorothioate, e.g., between 0 and 100%, 10 and 100%, 20 and 100%, 30 and 100%, 40 and 100%, 50 and 100%, 60 and 100% 70 and 100%, 80 and 100%, 90 and 100%, 0 and 90%, 0 and 80%, 0 and 70%, 0 and 60%, 0 and 50%, 0 and 40%, 0 and 30%, 0 and 20%, 0 and 10%, 10 and 90%, 20 and 80%, 30 and 70% 40 and 60%, 10 and 40%, 20 and 50%, and 60%, 40 and 70%, 50 and 80%, or 60 and 90% phosphorothioate linkages. Similarly, the internucleoside linkages may be between 0 and 100% phosphodiester linkages, e.g., between 0 and 100%, 10 and 100%, 20 and 100%, 30 and 100%, 40 and 100%, 50 and 100%, 60 and 100% 70 and 100%, 80 and 100%, 90 and 100%, 0 and 90%, 0 and 80%, 0 and 70%, 0 and 60%, 0 and 50%, 0 and 40%, 0 and 30%, 0 and 20%, 0 and 10%, 10 and 90%, 20 and 80%, 30 and 70%, 40 and 60%, 10 and 40%, 20 and 50%, 30 and 60%, 40 and 70%, 50 and 80%, or 60 and 90% phosphodiester linkages.

Specific examples of some potential oligomeric compounds useful in this invention include oligonucleotides containing modified e.g. non-naturally occurring internucleoside linkages. As defined in this specification, oligonucleotides having modified internucleoside linkages include internucleoside linkages that retain a phosphorus atom and internucleoside linkages that do not have a phosphorus atom. For the purposes of this specification, and as sometimes referenced in the art, modified oligonucleotides that do not have a phosphorus atom in their internucleoside backbone can also be considered to be oligonucleosides. In the C. elegans system, modification of the internucleotide linkage (phosphorothioate) did not significantly interfere with RNAi activity. Based on this observation, it is suggested that some compositions of the invention can also have one or more modified internucleoside linkages. A preferred phosphorus containing modified internucleoside linkage is the phosphorothioate internucleoside linkage. In some embodiments, the modified oligonucleotide backbones containing a phosphorus atom therein include, for example, phosphorothioates, phosphorodithioates, phosphotriesters, aminoalkylphosphotriesters, methyl and other alkyl phosphonates including 3′-alkylene phosphonates, 5′-alkylene phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates, and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. In some embodiments, the modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

siRNA Patterning

Nucleosides used in the invention tolerate a range of modifications in the nucleobase and sugar. A complete siRNA, single-stranded or double-stranded, may have 1, 2, 3, 4, 5, or more different nucleosides that each appear in the siRNA strand or strands once or more. The nucleosides may appear in a repeating pattern (e.g., alternating between two modified nucleosides) or may be a strand of one type of nucleoside with substitutions of a second type of nucleoside. Similarly, internucleoside linkages may be of one or more type appearing in a single- or double-stranded siRNA in a repeating pattern (e.g., alternating between two internucleoside linkages) or may be a strand of one type of internucleoside linkage with substitutions of a second type of internucleoside linkage. Though the siRNAs of the invention tolerate a range of substitution patterns, the following exemplify some preferred patterns in which A and B represent nucleosides of two types, and T and P represent internucleoside linkages of two types:

### Pattern 1: A-T-B-T-A-P-A-P-A-P-A-P-A-P-A-P-A-P-A-P-A-P-A-P-A-P-B-T-A-T-A-T-A-T-A-T-A-T-A-T A-T-A-T-A-P-A-P-A-P-A-P-A-P-A-P-A-P-A-P-A-P-A-P-A-P-A-T-A-T

### Pattern 2: A-T-A-T-A-P—B-P-B-P-B-P-A-P-A-P-A-P-A-P-A-P-A-P-A-P-B-T-A-T-A-T-A-T-A-T-A-T-A-T A-T-A-T-A-P-A-P-A-P-A-P-A-P-A-P-A-P-A-P-A-P-A-P-A-P-A-T-A-T

### Pattern 3: A-T-B-T-A-P—B-P-B-P-B-P-A-P-A-P-A-P-A-P-A-P-A-P-A-P-B-T-A-T-A-T-A-T-A-T-A-T-A-T A-T-A-T-A-P-A-P-A-P-A-P-A-P-A-P-A-P-A-P-A-P-A-P-A-P-A-T-A-T

### Pattern 4: A-T-B-T-A-P-A-P-A-P-A-P-A-P-A-P-A-P-A-P-A-P-A-P-A-P-B-T-A-T-A-T-A-T-A-T-A-T-A-T A-T-A-T-A-P-A-P-A-P-A-P—B-P-A-P-A-P—B-P-B-P-A-P-A-P-A-T-A-T

### Pattern 5: A-T-B-T-A-P-A-P-A-P—B-P-A-P-A-P-A-P-A-P-A-P-A-P-A-P-B-T-A-T-B-T-A-T-A-T-A-T-A-T A-T-A-T-A-P-A-P-A-P-A-P—B-P-A-P—B-P-B-P-B-P-A-P-A-P-A-T-A-T.

In some embodiments, T represents phosphorothioate, and P represents phosphodiester.

In some embodiments, the siRNA molecule of the disclosure features any one of the siRNA nucleotide modification patterns and/or internucleoside linkage modification patterns described in International Patent Application Publication Nos. WO 2016/161388 and WO 2020/041769, the disclosures of which are incorporated in their entirety herein. In some embodiments of the disclosure, the siRNA may contain an antisense strand including a region represented by Formula A-I, wherein Formula A-I is, in the 5′-to-3′ direction

A-B-(A′)ⱼ-C-P²-D-P¹-(C′-P¹)ₖ-C′ Formula A-I;

wherein A is represented by the formula C-P¹-D-P¹; each A′ is represented by the formula C-P²-D-P²; B is represented by the formula C-P²-D-P²-D-P²-D-P²; each C is a 2′-O-methyl (2′-O-Me) ribonucleoside; each C′, independently, is a 2′-O-Me ribonucleoside or a 2′-fluoro (2′-F) ribonucleoside; each D is a 2′-F ribonucleoside; each P¹ is a phosphorothioate internucleoside linkage; each P² is a phosphodiester internucleoside linkage; j is an integer from 1 to 7 (e.g., 1, 2, 3, 4, 5, 6, or 7); and k is an integer from 1 to 7 (e.g., 1, 2, 3, 4, 5, 6, or 7). In some embodiments, j is 4. In some embodiments, k is 4. In some embodiments, j is 4 and k is 4. The antisense is complementary (e.g., fully or partially complementary) to a target nucleic acid sequence.

In some embodiments, the antisense strand includes a structure represented by Formula A1, wherein Formula A1 is, in the 5′-to-3′ direction:

A-S-B-S-A-O-B-O-B-O-B-O-A-O-B-O-A-O-B-O-A-O-B-O-A-O-B-O-A-O-B-S-A-S-A-S-A-S-B-S-A Formula A1;

wherein A represents a 2′-O-Me ribonucleoside, B represents a 2′-F ribonucleoside, 0 represents a phosphodiester internucleoside linkage, and S represents a phosphorothioate internucleoside linkage.

In some embodiments of the disclosure, the siRNA may contain an antisense strand including a region represented by Formula A-II, wherein Formula A-II is, in the 5′-to-3′ direction:

A-B-(A),-C-P²-D-P¹-(C-P¹)k-C′ Formula A-II;

wherein A is represented by the formula C-P¹-D-P¹; each A′ is represented by the formula C-P²-D-P²; B is represented by the formula C-P²-D-P²-D-P²-D-P²; each C is a 2′-O-methyl (2′-O-Me) ribonucleoside; each C′, independently, is a 2′-O-Me ribonucleoside or a 2′-fluoro (2′-F) ribonucleoside; each D is a 2′-F ribonucleoside; each P¹ is a phosphorothioate internucleoside linkage; each P² is a phosphodiester internucleoside linkage; j is an integer from 1 to 7 (e.g., 1, 2, 3, 4, 5, 6, or 7); and k is an integer from 1 to 7 (e.g., 1, 2, 3, 4, 5, 6, or 7). In some embodiments, j is 4. In some embodiments, k is 4. In some embodiments, j is 4 and k is 4. The antisense is complementary (e.g., fully or partially complementary) to a target nucleic acid sequence.

In some embodiments of the disclosure, the antisense strand includes a structure represented by Formula A2, wherein Formula A2 is, in the 5′-to-3′ direction:

A-S-B-S-A-O-B-O-B-O-B-O-A-O-B-O-A-O-B-O-A-O-B-O-A-O-B-O-A-O-B-S-A-S-A-S-A-S-A-S-A Formula A2;

wherein A represents a 2′-O-Me ribonucleoside, B represents a 2′-F ribonucleoside, 0 represents a phosphodiester internucleoside linkage, and S represents a phosphorothioate internucleoside linkage.

In some embodiments of the disclosure, the sense strand includes a structure represented by Formula S-III, wherein Formula S-III is, in the 5′-to-3′ direction:

E-(A′)ₘ-F Formula S-III;

wherein E is represented by the formula (C-P¹)₂; F is represented by the formula (C-P²)₃-D-P¹-C-P¹-C, (C-P²)₃-D-P²-C-P²-C, (C-P²)₃-D-P¹-C-P¹-D, or (C-P²)₃-D-P²-C-P²-D; A′, C, D, P¹, and P² are as defined in Formula I; and m is an integer from 1 to 7 (e.g., 1, 2, 3, 4, 5, 6, or 7). In some embodiments, m is 4. The sense strand is complementary (e.g., fully or partially complementary) to the antisense strand.

In some embodiments of the disclosure, the sense strand includes a structure represented by Formula S1, wherein Formula S1 is, in the 5′-to-3′ direction:

A-S-A-S-A-O-B-O-A-O-B-O-A-O-B-O-A-O-B-O-A-O-A-O-A-O-B-S-A-S-A Formula S1;

wherein A represents a 2′-O-Me ribonucleoside, B represents a 2′-F ribonucleoside, 0 represents a phosphodiester internucleoside linkage, and S represents a phosphorothioate internucleoside linkage. In some embodiments of the disclosure, the sense strand includes a structure represented by Formula S2, wherein Formula S2 is, in the 5′-to-3′ direction:

A-S-A-S-A-O-B-O-A-O-B-O-A-O-B-O-A-O-B-O-A-O-A-O-A-O-B-O-A-O-A Formula S2;

wherein A represents a 2′-O-Me ribonucleoside, B represents a 2′-F ribonucleoside, 0 represents a phosphodiester internucleoside linkage, and S represents a phosphorothioate internucleoside linkage.

In some embodiments of the disclosure, the sense strand includes a structure represented by Formula S3, wherein Formula S3 is, in the 5′-to-3′ direction:

A-S-A-S-A-O-B-O-A-O-B-O-A-O-B-O-A-O-B-O-A-O-A-O-A-O-B-S-A-S-B Formula S3;

wherein A represents a 2′-O-Me ribonucleoside, B represents a 2′-F ribonucleoside, 0 represents a phosphodiester internucleoside linkage, and S represents a phosphorothioate internucleoside linkage.

In some embodiments of the disclosure, the sense strand includes a structure represented by Formula S4, wherein Formula S4 is, in the 5′-to-3′ direction:

A-S-A-S-A-O-B-O-A-O-B-O-A-O-B-O-A-O-B-O-A-O-A-O-A-O-B-O-A-O-B Formula S4;

wherein A represents a 2′-O-Me ribonucleoside, B represents a 2′-F ribonucleoside, 0 represents a phosphodiester internucleoside linkage, and S represents a phosphorothioate internucleoside linkage.

In some embodiments of the disclosure, the siRNA may contain an antisense strand including a region represented by Formula A-IV, wherein Formula A-IV is, in the 5′-to-3′ direction:

A-(A)ⱼ-C-P²-B-(C-P¹)ₖ-C′ Formula A-IV;

wherein A is represented by the formula C-P¹-D-P¹; each A′ is represented by the formula C-P²-D-P²; B is represented by the formula D-P¹-C-P¹-D-P¹; each C is a 2′-O-Me ribonucleoside; each C′, independently, is a 2′-O-Me ribonucleoside or a 2′-F ribonucleoside; each D is a 2′-F ribonucleoside; each P¹ is a phosphorothioate internucleoside linkage; each P² is a phosphodiester internucleoside linkage; j is an integer from 1 to 7 (e.g., 1, 2, 3, 4, 5, 6, or 7); and k is an integer from 1 to 7 (e.g., 1, 2, 3, 4, 5, 6, or 7). In some embodiments, j is 6. In some embodiments, k is 4. In some embodiments, j is 6 and k is 4. The antisense strand is complementary (e.g., fully or partially complementary) to a target nucleic acid. In some embodiments of the disclosure, the antisense strand includes a structure represented by Formula A3, wherein Formula A3 is, in the 5′-to-3′ direction:

A-S-B-S-A-O-B-O-A-O-B-O-A-O-B-O-A-O-B-O-A-O-B-O-A-O-B-O-A-O-B-S-A-S-B-S-A-S-A-S-A Formula A3;

wherein A represents a 2′-O-Me ribonucleoside, B represents a 2′-F ribonucleoside, 0 represents a phosphodiester internucleoside linkage, and S represents a phosphorothioate internucleoside linkage.

In some embodiments of the disclosure, the siRNA of the disclosure may have a sense strand represented by Formula S-V, wherein Formula S-V is, in the 5′-to-3′ direction:

E-(A′)ₘ-C-P²-F Formula S-V;

wherein E is represented by the formula (C-P¹)₂; F is represented by the formula D-P¹-C-P¹-C, D-P²-C-P²-C, D-P′-C-P′-D, or D-P²-C-P²-D; A′, C, D, P¹, and P² are as defined in Formula IV; and m is an integer from 1 to 7 (e.g., 1, 2, 3, 4, 5, 6, or 7). In some embodiments, m is 5. The sense strand is complementary (e.g., fully or partially complementary) to the antisense strand.

In some embodiments of the disclosure, the sense strand includes a structure represented by Formula S5, wherein Formula S5 is, in the 5′-to-3′ direction:

A-S-A-S-A-O-B-O-A-O-B-O-A-O-B-O-A-O-B-O-A-O-B-O-A-O-B-S-A-S-A Formula S5;

wherein A represents a 2′-O-Me ribonucleoside, B represents a 2′-F ribonucleoside, 0 represents a phosphodiester internucleoside linkage, and S represents a phosphorothioate internucleoside linkage.

In some embodiments of the disclosure, the sense strand includes a structure represented by Formula S6, wherein Formula S6 is, in the 5′-to-3′ direction:

A-S-A-S-A-O-B-O-A-O-B-O-A-O-B-O-A-O-B-O-A-O-B-O-A-O-B-O-A-O-A Formula S6;

wherein A represents a 2′-O-Me ribonucleoside, B represents a 2′-F ribonucleoside, 0 represents a phosphodiester internucleoside linkage, and S represents a phosphorothioate internucleoside linkage.

In some embodiments of the disclosure, the sense strand includes a structure represented by Formula S7, wherein Formula S7 is, in the 5′-to-3′ direction:

A-S-A-S-A-O-B-O-A-O-B-O-A-O-B-O-A-O-B-O-A-O-B-O-A-O-B-S-A-S-B Formula S7;

wherein A represents a 2′-O-Me ribonucleoside, B represents a 2′-F ribonucleoside, 0 represents a phosphodiester internucleoside linkage, and S represents a phosphorothioate internucleoside linkage.

In some embodiments of the disclosure, the sense strand includes a structure represented by Formula S8, wherein Formula S8 is, in the 5′-to-3′ direction:

A-S-A-S-A-O-B-O-A-O-B-O-A-O-B-O-A-O-B-O-A-O-B-O-A-O-B-O-A-O-B Formula S8;

wherein A represents a 2′-O-Me ribonucleoside, B represents a 2′-F ribonucleoside, 0 represents a phosphodiester internucleoside linkage, and S represents a phosphorothioate internucleoside linkage.

In some embodiments of the disclosure, the siRNA may contain an antisense strand including a region represented by Formula A-VI, wherein Formula A-VI is, in the 5′-to-3′ direction:

A-Bⱼ-E-Bₖ-E-F-Gₗ-D-P¹-C′ Formula A-VI;

wherein A is represented by the formula C-P¹-D-P¹; each B is represented by the formula C-P²; each C is a 2′-O-Me ribonucleoside; each C′, independently, is a 2′-O-Me ribonucleoside or a 2′-F ribonucleoside; each D is a 2′-F ribonucleoside; each E is represented by the formula D-P²-C-P²; F is represented by the formula D-P¹-C-P¹; each G is represented by the formula C-P¹; each P¹ is a phosphorothioate internucleoside linkage; each P² is a phosphodiester internucleoside linkage; j is an integer from 1 to 7 (e.g., 1, 2, 3, 4, 5, 6, or 7); k is an integer from 1 to 7 (e.g., 1, 2, 3, 4, 5, 6, or 7); and I is an integer from 1 to 7 (e.g., 1, 2, 3, 4, 5, 6, or 7). In some embodiments, j is 3. In some embodiments, k is 6. In some embodiments, I is 2. In some embodiments, j is 3, k is 6, and I is 2. The antisense strand is complementary (e.g., fully or partially complementary) to a target nucleic acid.

In some embodiments of the disclosure, the antisense strand includes a structure represented by Formula A4, wherein Formula A4 is, in the 5′-to-3′ direction:

A-S-B-S-A-O-A-O-A-O-B-O-A-O-A-O-A-O-A-O-A-O-A-O-A-O-B-O-A-O-B-S-A-S-A-S-A-S-B-S-A Formula A4;

wherein A represents a 2′-O-Me ribonucleoside, B represents a 2′-F ribonucleoside, 0 represents a phosphodiester internucleoside linkage, and S represents a phosphorothioate internucleoside linkage.

In some embodiments of the disclosure, the siRNA may contain a sense strand including a region represented by Formula S-VII, wherein Formula S-VII is, in the 5′-to-3′ direction:

H-Bₘ-Iₙ-A′-Bₒ-H-C Formula S-VII;

wherein A′ is represented by the formula C-P²-D-P²; each H is represented by the formula (C-P¹)₂; each I is represented by the formula (D-P²); B, C, D, P¹, and P² are as defined in Formula VI; m is an integer from 1 to 7 (e.g., 1, 2, 3, 4, 5, 6, or 7); n is an integer from 1 to 7 (e.g., 1, 2, 3, 4, 5, 6, or 7); and o is an integer from 1 to 7 (e.g., 1, 2, 3, 4, 5, 6, or 7). In some embodiments, m is 3. In some embodiments, n is 3. In some embodiments, o is 3. In some embodiments, m is 3, n is 3, and o is 3. The sense strand is complementary (e.g., fully or partially complementary) to the antisense strand.

In some embodiments of the disclosure, the sense strand includes a structure represented by Formula S9, wherein Formula S9 is, in the 5′-to-3′ direction:

A-S-A-S-A-O-A-O-A-O-B-O-B-O-B-O-A-O-B-O-A-O-A-O-A-O-A-S-A-S-A Formula S9;

wherein A represents a 2′-O-Me ribonucleoside, B represents a 2′-F ribonucleoside, 0 represents a phosphodiester internucleoside linkage, and S represents a phosphorothioate internucleoside linkage.

In some embodiments of the disclosure, the siRNA may contain an antisense strand including a region that is represented by Formula VIII:

5′ Phosphorus Stabilizing Moiety

To further protect the siRNA from degradation a 5′-phosphorus stabilizing moiety may be employed. A 5′-phosphorus stabilizing moiety replaces the 5′-phosphate to prevent hydrolysis of the phosphate. Hydrolysis of the 5′-phosphate prevents binding to RISC, a necessary step in gene silencing. Any replacement for phosphate that does not impede binding to RISC is contemplated in this disclosure. In some embodiments, the replacement for the 5′-phosphate is also stable to in vivo hydrolysis. Each siRNA strand may independently and optionally employ any suitable 5′-phosphorus stabilizing moiety.

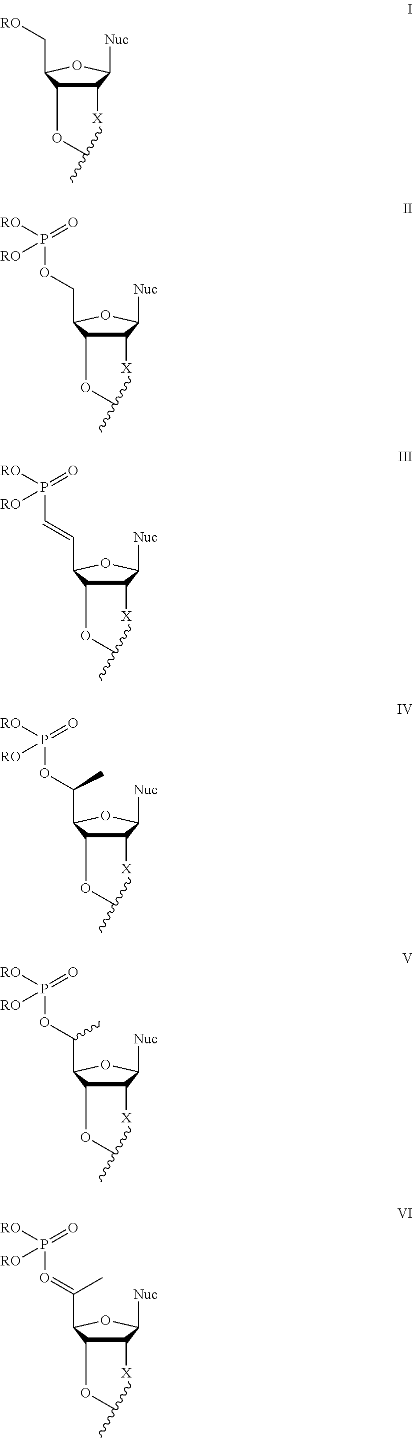

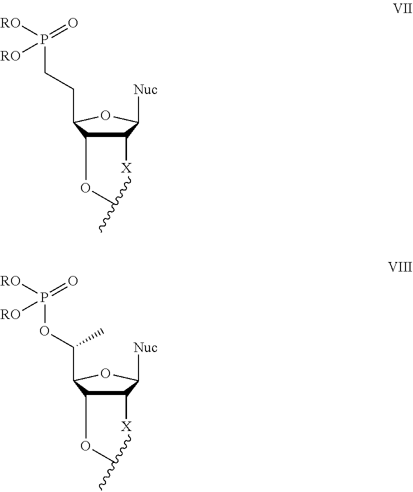

Some exemplary endcaps are demonstrated in Formula I-VIII. Nuc in Formula I-VIII represents a nucleobase or nucleobase derivative or replacement as described herein. X in Formula I-VIII represents a 2′-modification as described herein. Some embodiments employ hydroxy as in Formula I, phosphate as in Formula II, vinylphosphonates as in Formula III, and VI, 5′-methylsubstitued phosphates as in Formula IV, VI, and VIII, or methylenephosphonates as in Formula VII, vinyl 5′-vinylphosphonate as a 5′-phosphorus stabilizing moiety as demonstrated in Formula III.

siRNA Branching

Branching of the siRNA molecules is a key feature in the present invention. The siRNA molecule may not be branched, or may be dibranched, tribranched, or tetrabranched, connected through a linker. Each main branch may be further branched to allow for 2, 3, 4, 5, 6, 7, or 8 separate RNA single- or double-strands. The branch points on the linker may stem from the same atom, or separate atoms along the linker. Some exemplary embodiments are listed in Table 1, where L represent a linker, and X represents any atom suitable to the siRNA molecule branch points:

TABLE 1
Branched siRNA structures

| Dibranched | Tribranched | Tetrabranched |
|---|---|---|
| RNA—L—RNA | 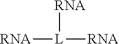 | 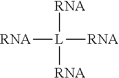 |
| 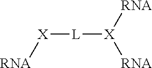 | 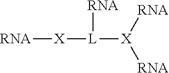 | 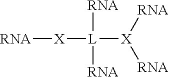 |
| 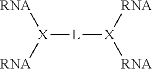 | 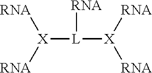 | 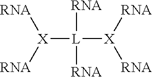 |
| | 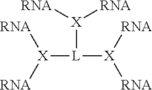 | 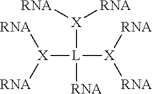 |
| | | 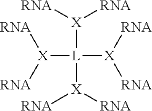 |

Linkers

Multiple strands of siRNA described herein may be covalently attached by way of a linker. The effect of this branching improves, inter alia, cell permeability allowing better access into microglia in the CNS. Any linking moiety may be employed which is not incompatible with the siRNAs of the present invention. Exemplary linkers include ethylene glycol chains of 2 to 10 subunits (e.g., 2, 3, 4, 5, 6, 7, 8, 9, or 10 subunits), alkyl chains, carbohydrate chains, block copolymers, peptides, RNA, DNA, and others. In some embodiments, any carbon or oxygen atom of the linker is optionally replaced with a nitrogen atom, bears a hydroxyl substituent, or bears an oxo substituent. In some embodiments, the linker is a poly-ethylene glycol (PEG) linker. The PEG linkers suitable for use with the disclosed compositions and methods include linear or non-linear PEG linkers. Examples of non-linear PEG linkers include branched PEGs, linear forked PEGs, or branched forked PEGs.

PEG linkers of various weights may be used with the disclosed compositions and methods. For example, the PEG linker may have a weight that is between 5 and 500 Daltons. In some embodiments, a PEG linker having a weight that is between 500 and 1,000 Dalton may be used. In some embodiments, a PEG linker having a weight that is between 1,000 and 10,000 Dalton may be used. In some embodiments, a PEG linker having a weight that is between 200 and 20,000 Dalton may be used. In some embodiments, the linker is covalently attached to a sense strand of the siRNA. In some embodiments, the linker is covalently attached to an antisense strand of the siRNA. In some embodiments, the PEG linker is a triethylene glycol (TrEG) linker. In some embodiments, the PEG linker is a tetraethylene linker (TEG).

In some embodiments, the linker is an alkyl chain linker. In some embodiments, the linker is a peptide linker. In some embodiments, the linker is a RNA linker. In some embodiments, the linker is a DNA linker.

Linkers may covalently link 2, 3, 4, or 5 unique siRNA strands. The linker may covalently bind to any part of the siRNA oligomer. In some embodiments, the linker attaches to the 3′ end of nucleosides of each siRNA strand. In some embodiments, the linker attaches to the 5′ end of nucleosides of each siRNA strand. In some embodiments, the linker attaches to a nucleoside of an siRNA strand (e.g., sense or antisense strand) by way of a covalent bond-forming moiety. In some embodiments, the covalent-bond-forming moiety is selected from the group consisting of an alkyl, ester, amide, carbonate, carbamate, triazole, urea, formacetal, phosphonate, phosphate, and phosphate derivative (e.g., phosphorothioate, phosphoramidate, etc.).

In some embodiments, the linker has a structure of Formula L1, as is shown below:

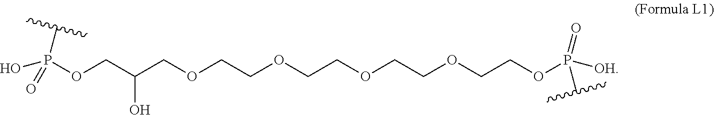 [SMILES: *P(=O)(O)OCCOCCOCCOCCOCC(O)COP(*)(=O)O]

In some embodiments, the linker has a structure of Formula L2, as is shown below:

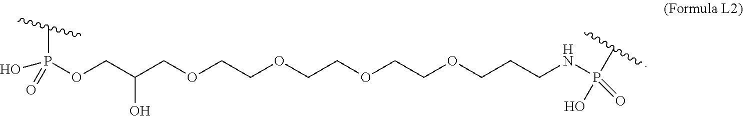

In some embodiments, the linker has a structure of Formula L3, as is shown below:

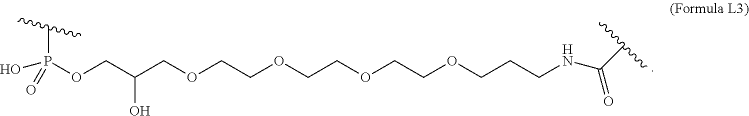

In some embodiments, the linker has a structure of Formula L4, as is shown below:

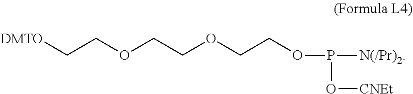

In some embodiments, the linker has a structure of Formula L5, as is shown below:

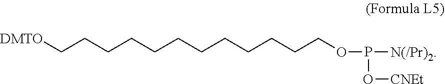

In some embodiments, the linker has a structure of Formula L6, as is shown below:

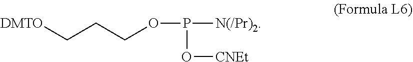

In some embodiments, the linker has a structure of Formula L7, as is shown below:

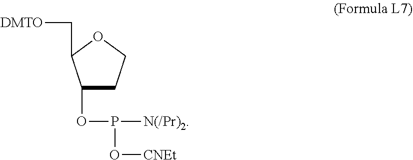

In some embodiments, the linker has a structure of Formula L8, as is shown below:

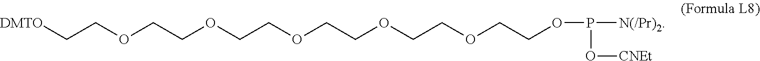

In some embodiments, the linker has a structure of Formula L9, as is shown below:

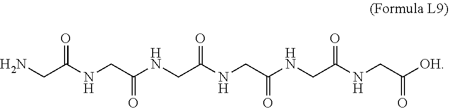 [SMILES: NCC(=O)NCC(=O)NCC(=O)NCC(=O)NCC(=O)NCC(=O)O]

In some embodiments, the selection of a linker for use with one or more of the branched siRNA molecules disclosed herein may be based on the hydrophobicity of the linker, such that, e.g., desirable hydrophobicity is achieved for the one or more branched siRNA molecules of the disclosure. For example, a linker containing an alkyl chain may be used to increase the hydrophobicity of the branched siRNA molecule as compared to a branched siRNA molecule having a less hydrophobic linker or a hydrophilic linker.

### Methods of Treatment

The invention provides methods of treating a subject in need of gene silencing. The gene silencing may be performed in order to silence defective or overactive microglial genes, silence negative regulators of microglial genes with reduced expression and/or activity, silence wild type microglial genes with an activating role in a pathway(s) that increases expression and/or activity of a disease driver gene, silence splice isoforms of a microglial gene(s) that, when selectively knocked down, may elevate total expression and/or activity of the gene(s), among other reasons, so long as the goal is to restore genetic and biochemical pathway activity from a disease state towards a healthy state. The active compound can be administered in any suitable dose. The actual dosage amount of a composition of the present invention administered to a patient can be determined by physical and physiological factors such as body weight, severity of condition, previous or concurrent therapeutic interventions, idiopathy of the patient and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject. Administration may occur any suitable number of times per day, and for as long as necessary. Subjects may be adult or pediatric humans, with or without comorbid diseases.

### Diseases

The methods of the invention feature delivering a branched siRNA molecule to a microglial cell in a subject in need of microglial gene silencing. Subjects in need of microglial gene silencing may be suffering from neurodegenerative diseases in which neuroinflammation is a primary component of the disease pathology (e.g., Alzheimer's disease, amyotrophic lateral sclerosis, Parkinson's disease, frontotemporal dementia, Huntington's disease, multiple sclerosis, or progressive supranuclear palsy).

Alzheimer's Disease

Alzheimer's disease (AD) is a late-onset neurodegenerative disorder responsible for the majority of dementia cases in the elderly. AD patients suffer from a progressive cognitive decline characterized by symptoms including an insidious loss of short- and long-term memory, attention deficits, language-specific problems, disorientation, impulse control, social withdrawal, anhedonia, and other symptoms. Distinguishing neuropathological features of AD are extracellular aggregates of amyloid-6 plaques and neurofibrillary tangles composed of hyperphosphorylated microtubule-associated tau proteins. Accumulation of these aggregates is associated with neuronal loss and atrophy in a number of brain regions including the frontal, temporal, and parietal lobes of the cerebral cortex as well as subcortical structures like the basal forebrain cholinergic system and the locus coeruleus within the brainstem. AD is also associated with increased neuroinflammation characterized by reactive gliosis and elevated levels of pro-inflammatory cytokines.

Amyotrophic Lateral Sclerosis

Amyotrophic Lateral Sclerosis (ALS) is a fast-progressing fatal neurodegenerative disease that affects motor neurons both in the brain and spinal cord, consequently resulting in paralysis of voluntary muscles at later stages of disease. ALS affects about 6 persons per 100,000 people and typically leads to death within 3 to 5 years after the onset of symptoms, with no cure yet available. ALS leads to muscle weakness, atrophy, and muscle spasms as a result of degeneration of upper and lower motor neurons. Cognitive and behavioral dysfunction (e.g., language dysfunction, executive dysfunction, social cognition, and verbal memory dysfunction), and frontotemporal dementia are all possible symptoms of ALS.

Parkinson's Disease

PD is a progressive disorder that affects movement, and it is recognized as the second most common neurodegenerative disease after Alzheimer's disease. Common symptoms of PD include resting tremor, rigidity, and bradykinesia, and non-motor symptoms, such as depression, constipation, pain, sleep disorders, genitourinary problems, cognitive decline, and olfactory dysfunction, are also increasingly being associated with PD. A key feature of PD is the death of dopaminergic neurons in the substantia nigra pars compacta, and, for that reason, most current treatments for PD focus on increasing dopamine. Another well-known neuropathological hallmark of PD is the presence of Lewy bodies containing α-synuclein in brain regions affected by PD, which are thought to contribute to the disease.

PD is thought to result from a combination of genetic and environmental risk factors. There is no single gene responsible for all Parkinson's disease cases, and the vast majority of PD cases seem to be sporadic and not directly inherited. Mutations in the genes encoding parkin, PTEN-induced putative kinase 1 (PINK1), leucine-rich repeat kinase 2 (LRRK2), and Parkinsonism-associated deglycase (DJ-1) have been found to be associated with PD, but they represent only a small subset of the total number of PD cases. Occupational exposure to some pesticides and herbicides has also been proposed as a risk factor for PD. The synthetic neurotoxin MPTP can cause Parkinsonism, but its use is extremely rare.

Frontotemporal Dementia

Frontotemporal dementia (FTD; also known as frontotemporal lobar degeneration (FTLD)) is a clinical syndrome characterized by progressive neurodegeneration in the frontal and temporal lobes of the cerebral cortex. The manifestation of FTD is complex and heterogeneous, and may present as one of three clinically distinct variants including: 1) behavioral-variant frontotemporal dementia (BVFTD), characterized by changes in behavior and personality, apathy, social withdrawal, perseverative behaviors, attentional deficits, disinhibition, and a pronounced degeneration of the frontal lobe; 2) semantic dementia (SD), characterized by fluent, anomic aphasia, progressive loss of semantic knowledge of words, objects, and concepts and a pronounced degeneration of the anterior temporal lobes. Furthermore, SD variant of FTD exhibit a flat affect, social deficits, perseverative behaviors, and disinhibition; or 3) progressive nonfluent aphasia; characterized by motor deficits in speech production, reduced language expression, and pronounced degeneration of the perisylvian cortex. Neuronal loss in brains of FTD patients is associated with one of three distinct neuropathologies: 1) the presence of tau-positive neuronal and glial inclusions; 2) ubiquitin (ub)-positive and TAR DNA-binding protein 43 (TDP43)-positive, but tau-negative inclusions; or 3) ub and fused in sarcoma (FUS)-positive, but tau and TDP-43-negative inclusions. These neuropathologies are considered to be important in the etiology of FTD.

Nearly half of FTD patients have a first-degree family member with dementia, ALS, or Parkinson's disease, suggesting a strong genetic link to the cause of the disease. A number of mutations in chromosome 17q21 have been linked to FTD presentation.

Huntington's Disease

Huntington's Disease (HD) is an example of a trinucleotide repeat expansion disorder. This class of disorders involve the localized expansion of unstable repeats of sets of three nucleotides and can result in loss of function of a gene in which the expanded repeat is found, a gain of toxic function, or both. Trinucleotide repeats can be located in any part of the gene, including coding and non-coding regions. Repeats located within coding regions typically involve a repeated glutamine encoding triplet (CAG) or an alanine encoding triplet (CGA). Expanded repeat regions within non-coding sequences can lead to aberrant expression of the gene, while expanded repeats within coding regions (also known as codon reiteration disorders) may cause protein mis-folding and aggregation. Typically, regions of wild-type genes contain a variable number of repeat sequences in the normal population, but in the afflicted populations, the number of repeats can increase from a doubling to a log order increase in the number of repeats. In HD, repeats are inserted within the N-terminal coding region of the large cytosolic protein Huntingtin (Htt). Normal Htt alleles contain 15-20 CAG repeats, while alleles containing 35 or more repeats can be considered to confer a risk for developing the disease. Alleles containing 36-39 repeats are considered incompletely penetrant, and those individuals harboring those alleles may or may not develop the disease (or exhibit delayed presentation later in life), while alleles containing 40 repeats or more are considered completely penetrant. Those individuals with juvenile onset HD (<21 years of age) are often found to have 60 or more CAG repeats.

Multiple Sclerosis

Multiple sclerosis (MS) is the most common demyelinating disease of the CNS affecting young adults (disease onset between 20 to 40 years of age) and is the third leading cause for disability after trauma and rheumatic diseases in the US.

MS patients present with destruction of myelin, death of oligodendrocytes, and axonal loss. The main pathologic finding in MS is the presence of infiltrating mononuclear cells, predominantly T lymphocytes and macrophages, which breach the blood brain barrier and induce active inflammation within the CNS. The neurological symptoms that characterize MS include complete or partial vision loss, diplopia, sensory symptoms, motor weakness that can progress to complete paralysis, bladder dysfunction, and cognitive deficits. The associated inflammatory foci lead to myelin destruction, plaques of demyelination, gliosis, and axonal loss within the brain and spinal cord and are the primary drivers of the clinical manifestations of neurological disability.

The etiology of MS is not fully understood. The disease develops in genetically predisposed subjects exposed to yet undefined environmental factors and the pathogenesis involves autoimmune mechanisms associated with autoreactive T cells against myelin antigens. It is well established that not one dominant gene determines genetic susceptibility to develop MS, but rather many genes, each with different influence, are involved. The detailed molecular mechanisms underlying MS etiology are still to be elucidated.

Progressive Supranuclear Palsy

Progressive supranuclear palsy (PSP), a progressive and fatal tauopathy, represents ˜10% of all Parkinsonian cases in the US. PSP patients have a variety of motor disorders, including postural instability, falls, abnormalities in gait, bradykinesia, vertical gaze paralysis, pseudobulbar paralysis, and axial stiffness without limb stiffness, in addition to cognitive impairments such as apathy, loss of executive function, and reduced fluency. Neuropathology of PSP is characterized by an accumulation of tau protein, which is associated with abnormal intracellular microtubules, resulting in insoluble filament deposits. The neuropathological presentation of PSP neurodegeneration is located in the subcortical regions, including substantia nigra, globus pallidus, and subthalamic nucleus. PSP neurodegeneration is characterized by the destruction of tissues and cytokine profiles of activated microglia and astrocytes.

There are currently no disease-modifying treatments for PSP. The current standard of care is palliative. Patients in the advanced stages of the disease often have feeding tubes inserted to avoid choking hazards and to provide nutrition. Although therapies are available to decrease some symptoms of PSP, none protect the brain from neurodegeneration. Current medications to treat symptoms of PSP include dopamine agonists, tricyclic antidepressants, methysergide, onabotulinumtoxin A (to treat muscle stiffness in the face). However, as the disease progresses and symptoms worsen, medications may fail to adequately decrease symptoms.

### Gene Targets

The methods of the invention feature delivering a branched siRNA molecule to a microglial cell in a subject in need of microglial gene silencing. Patients in need of microglial gene silencing may have dysregulated expression and/or activity of a gene selected from the group consisting of ABCA7, ABI3, ADAM10, APOC1, APOE, AXL, BIN1, C1QA, C3, C9ORF72, CASS4, CCL5, CD2AP, CD33, CD68, CLPTM1, CLU, CR1, CSF1, CST7, CTSB, CTSD, CTSL, CXCL10, CXCL13, DSG2, ECHDC3, EPHA1, FABP5, FERMT2, FTH1, GNAS, GRN, HBEGF, HLA-DRB1, HLA-DRB5, IFIT1, IFIT3, IFITM3, IFNAR1, IFNAR2, IGF1, IL10RA, IL1A, IL1B, IL1RAP, INPP5D, ITGAM, ITGAX, LILRB4, LPL, MEF2C, MMP12, MS4A4A, MS4A6A, NLRP3, NME8, NOS2, PICALM, PILRA, PLCG2, PTK2B, SCIMP, SLC24A4, SORL1, SPI1, SPP1, SPPL2A, TBK1, TNF, TREM2, TREML2, TYROBP, and ZCVVPW1 gene.

In some embodiments, the patient in need of microglial gene silencing may require silencing of any one of the genes selected from the group consisting of APOE, BIN1, C1QA, C3, C9ORF72, CCL5, CD33, CLU/APOJ, CR1, CXCL10, CXCL13, IFIT1, IFIT3, IFITM3, IFNAR1, IFNAR2, IL10RA, IL1A, IL1B, IL1RAP, INPP5D, ITGAM, MEF2C, MMP12, NLRP3, NOS2, PILRA, PLCG2, PTK2B, SLC24A4, TBK1, and TNF.

### Pharmaceutical Compositions

The branched siRNA molecules in the present invention can be formulated into a pharmaceutical composition for administration to a subject in a biologically compatible form suitable for administration in vivo. Accordingly, in one aspect, the present invention provides a pharmaceutical composition containing a branched siRNA in admixture with a suitable diluent, carrier, or excipient. The siRNA can be administered, for example, orally or by intravenous injection.

Conventional procedures and ingredients for the selection and preparation of suitable formulations are described, for example, in Remington: The Science and Practice of Pharmacy (2012, 22 nd ed.) and in The United States Pharmacopeia: The National Formulary (2015, USP 38 NF 33).

Under ordinary conditions of storage and use, a pharmaceutical composition may contain a preservative, e.g., to prevent the growth of microorganisms. Pharmaceutical compositions may include sterile aqueous solutions, dispersions, or powders, e.g., for the extemporaneous preparation of sterile solutions or dispersions. In all cases the form may be sterilized using techniques known in the art and may be fluidized to the extent that may be easily administered to a subject in need of treatment.

A pharmaceutical composition may be administered to a subject, e.g., a human subject, alone or in combination with pharmaceutically acceptable carriers, as noted herein, the proportion of which may be determined by the solubility and/or chemical nature of the compound, chosen route of administration, and standard pharmaceutical practice.

### Regimens

A physician having ordinary skill in the art can readily determine an effective amount of siRNA for administration to a mammalian subject (e.g., a human) in need thereof. For example, a physician could start prescribing doses of a siRNA of the invention at levels lower than that required in order to achieve the desired therapeutic effect and gradually increase the dosage until the desired effect is achieved. Alternatively, a physician may begin a treatment regimen by administering a siRNA at a high dose and subsequently administer progressively lower doses until a therapeutic effect is achieved (e.g., a reduction in expression of a target gene sequence). In general, a suitable daily dose of a siRNA of the invention will be an amount of the siRNA which is the lowest dose effective to produce a therapeutic effect. A single-strand or double-strand siRNA of the invention may be administered by injection, e.g., intrathecally, intracerebroventricularly, or intrastriatally. A daily dose of a therapeutic composition of a siRNA of the invention may be administered as a single dose or as two, three, four, five, six or more doses administered separately at appropriate intervals throughout the day, week, month, or year, optionally, in unit dosage forms. While it is possible for a siRNA of the invention to be administered alone, it may also be administered as a pharmaceutical formulation in combination with excipients, carriers, and optionally, additional therapeutic agents.

### Routes of Administration

The method of the invention contemplates any route of administration tolerated by the therapeutic composition. Some embodiments of the method include injection intrathecally, intracerebroventricularly, or intrastriatally.

Intrathecal injection is the direct injection into the spinal column or subarachnoid space. By injecting directly into the CSF of the spinal column the siRNA molecule of the invention has direct access to microglia in the spinal column and a route to access the microglia in the brain by bypassing the blood brain barrier.

Intracerebroventricular (ICV) injection is a method to directly inject into the CSF of the cerebral ventricles. Similar to intrathecal injection, ICV is a method of injection which bypasses the blood brain barrier. Using ICV allows the advantage of access to the microglia of the brain and spinal column without the danger of the therapeutic being degraded in the blood.

Intrastriatal injection is the direct injection into the striatum, or corpus striatum. The striatum is an area in the subcortical basal ganglia in the brain. Injecting into the striatum bypasses the blood brain barrier and the pharmacokinetic challenges of injection into the blood stream and allows for direct access to the microglia of the brain and spinal column.

### EXAMPLES

### Example 1. Protocol for Uptake of Di-siRNA in Microglia of Non-Human Primates

The experiments described in this example were conducted to assess the ability of branched siRNA molecules to permeate the central nervous system and internalize within microglial cells. To this end, a branched siRNA compound targeting the huntingtin (HTT) gene and conjugated to a fluorescent dye (Cy3) was first injected into the cerebrospinal fluid via intrathecal injection into non-human primates (NHP; cynomolgus macaque). Central nervous system tissue samples were later obtained from the animals. To assess the extent to which the branched siRNA molecules were internalized by microglial cells, the tissue samples were stained using fluorescent-labeled antibodies that are specific for markers expressed in certain cell types (e.g., microglia). Fluorescence microscopy was then utilized to determine the degree of colocalization of the Cy3-labeled branched siRNA molecules and antibody-labeled microglial cells, which served as an indicator of microglial uptake. These experiments, and their results, are described in further detail below:

Paraffin embedded CNS tissue slides were tested. A dose of fluorescent labeled branched siRNA was administered to a NHP (cynomolgus macaque) via intrathecal injection. 48 hours after injection a distribution study was done. The control was an uninjected NHP. NHP tissues for imaging were post-fixed for 48-72 hours in 4% PFA at 5±3° C., and then transferred to PBS. All tissues were paraffin-embedded and sliced into 4 μm sections and mounted on slides for immunofluorescence staining. Subsequently, sections were deparaffinized and subjected to antigen retrieval. Samples were deparaffanized by two changes of xylene, 5 minutes each, then 50% xylene+50% ethanol (100%) for 5 minutes. Samples were hydrated by two changes of 100% ethanol for 3 minutes each, 90%, 80%, 70% and then 50% ethanol for 3 minutes each, followed by distilled water rinse. Antigen retrieval was carried out using 150 mL of Tris-EDTA buffer (pH9), placing the staining dish in a pressure cooker (containing 1200 mL DDH₂O) for 10 minutes, allowing the slides to cool to room temperature, followed by section-wise rinsing with H₂O and TBST. Sections were blocked with Background Terminator Blocking Reagent and the slides were then incubated with the primary antibody against the microglial-specific gene, Iba-1, for 1.5 hours at room temperature, followed by treatment with a secondary antibody labeled with Alexa Flour 488 (Alexa-488). Alexa-488 was used to visualize Iba-1 antibody. DAPI was used to visualize cell nuclei. Tissues were washed three times for 5 min with TBS-Tween 20. Fluoromount-G was used to place glass coverslips, and slides were left to dry at 4° C. overnight protected from light. Olympus VS200 slide scanner was used to acquire immunofluorescent images of brain and spinal cord (20× objective). Images within each imaging channel were acquired under the same settings for light intensity and exposure times.

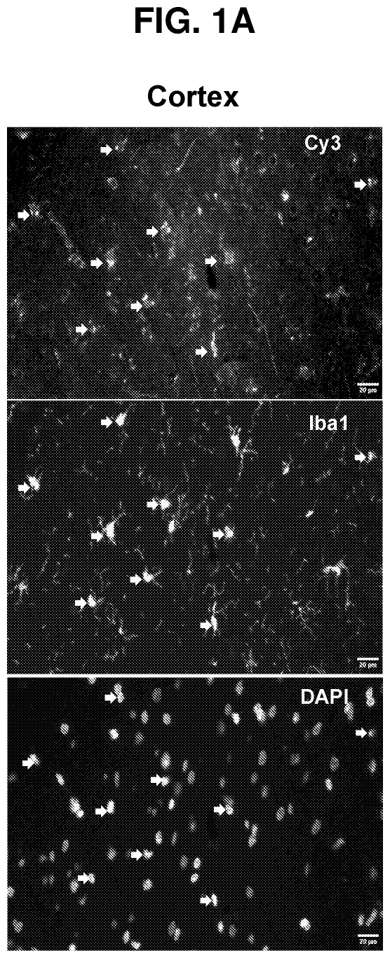

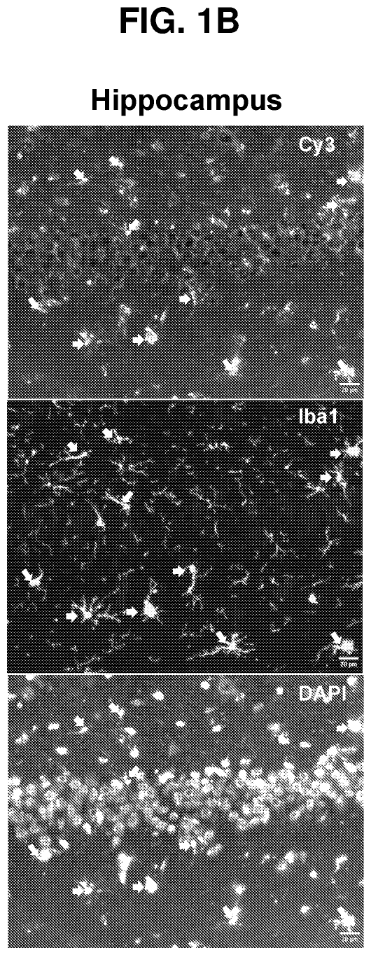

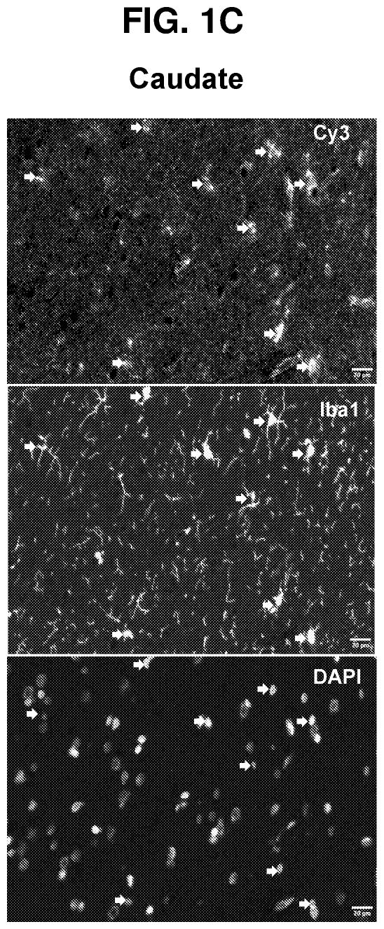

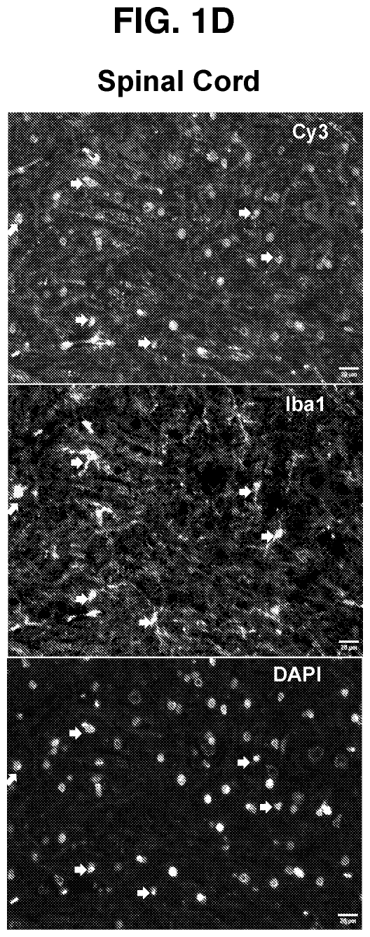

Colocalization of DAPI stained nuclei, Alexa-488-labeled Iba-1 antibody, and Cy3-labeled siRNA was observed across all tested brain and spinal cord tissues of cynomolgus macaques, indicating microglial cell penetration and/or uptake of the branched di-siRNA. Control experiments included uninjected NHP control (no Cy3-siRNA), non-specific primary antibody (isotype antibody control), and no secondary antibody (no Alexa Fluor 488 reagent). Robust colocalization was observed in the cortex (FIG. 1A), hippocampus (FIG. 1B), caudate nucleus (FIG. 1C), and spinal cord (FIG. 1D). Controls showed no co-localization of Cy3 and Alexa Fluor 488 signals, indicating specificity of detection of microglial uptake (not shown).

These results demonstrate that the ds-siRNA agents of the present disclosure are capable of being internalized by microglial cells of CNS tissues, including brain and spinal cord, and support the use of such agents for treatment of neurological conditions, such as Alzheimer's disease or amyotrophic lateral sclerosis.

### Example 2. Method of Treating a Patient with Alzheimer's Disease

A subject diagnosed with Alzheimer's disease is treated with a dose and frequency determined by a practitioner (e.g., three times daily, twice daily, once daily, once weekly, once monthly, bi-monthly, once every 4 months, once every 5 months, once every 6 months, once every 7 months, once every 8 months, once every 9 months, once every 10 months, once every 11 months, or annually). Dosage and frequency are determined based on the subject's height, weight, age, sex, and other disorders.

The branched siRNA is selected by the practitioner for compatibility with the disease and subject. Single- or double-stranded branched siRNA are available for selection. The siRNA chosen has an antisense strand, and in the case of double-stranded siRNA, a sense strand with a sequence and RNA modifications (e.g., natural and non-natural internucleoside linkages, modified sugars, and 5′-phosphorus stabilizing moieties) best suited to the patient and the disease being targeted (e.g., PSM-A-T-B-T-A-P-B-P-A-P-B-P-A-P-B-P-A-P-B-P-A-P-B-P-A-P-B-T-A-T-B-T-A-T-B-T-A-T-B-T B-T-A-T-B-P-A-P-B-P-A-P-B-P-A-P-B-P-A-P-B-P-A-P-B-P-A-T-B-T where A and B are different nucleosides, T is phosphorothioate, P is a phosphodiester, and PSM is a 5′-phosphorus stabilizing moiety).

The branched siRNA is delivered by the route best suited the patient and condition (e.g., intrathecally, intracerebroventricularly, or intrastriatally), at a rate tolerable to the patient until the subject has reached a maximum tolerated dose, or until the symptoms of the disease are ameliorated satisfactorily.

### Example 3. Method of Treating a Patient with Amyotrophic Lateral Sclerosis

A subject diagnosed with Amyotrophic Lateral Sclerosis is treated with a dose and frequency determined by a practitioner (e.g., three times daily, twice daily, once daily, once weekly, once monthly bi-monthly, once every 4 months, once every 5 months, once every 6 months, once every 7 months, once every 8 months, once every 9 months, once every 10 months, once every 11 months, or annually). Dosage and frequency are determined based on the subject's height, weight, age, sex, and other disorders.

The branched siRNA is selected by the practitioner for compatibility with the disease and subject. Single- or double-stranded branched siRNA are available for selection. The siRNA chosen has an antisense strand, and in the case of double-stranded siRNA, a sense strand with a sequence and RNA modifications (e.g., natural and non-natural internucleoside linkages, modified sugars, and 5′-phosphorus stabilizing moieties) best suited to the patient and the disease being targeted (e.g., PSM-A-T-B-T-A-P-B-P-A-P-B-P-A-P-B-P-A-P-B-P-A-P-B-P-A-P-B-T-A-T-B-T-A-T-B-T-A-T-B-T B-T-A-T-B-P-A-P-B-P-A-P-B-P-A-P-B-P-A-P-B-P-A-P-B-P-A-T-B-T where A and B are different nucleosides, T is phosphorothioate, P is a phosphodiester, and PSM is a 5′-phosphorus stabilizing moiety).

The branched siRNA is delivered by the route best suited the patient and condition (e.g., intrathecally, intracerebroventricularly, or intrastriatally), at a rate tolerable to the patient until the subject has reached a maximum tolerated dose, or until the symptoms of the disease are ameliorated satisfactorily.

### Specific Embodiments

Some specific embodiments are listed below. The below enumerated embodiments should not be construed to limit the scope of the invention, rather, the below are presented as some examples of the utility of the invention.
E1. A method of delivering a branched small interfering RNA (siRNA) molecule to a microglial cell in a subject in need of microglial gene silencing, the method comprising administering the branched siRNA molecule to the central nervous system of the subject.
E2. The method of E1, wherein the subject has been diagnosed as having a disease associated with expression of a dysregulated microglial gene or dysregulated microglial gene network.
E3. The method of E2, wherein the dysregulated microglial gene exhibits increased expression and/or activity in microglial cells of the subject as compared to the expression and/or activity of the microglial gene in microglial cells of a reference subject.
E4. The method of E2, wherein the dysregulated microglial gene exhibits reduced expression and/or activity in microglial cells of the subject as compared to the expression and/or activity of the microglial gene in microglial cells of a reference subject.
E5. The method of any one of E1-E4, wherein the delivering of the branched siRNA molecule to the subject results in silencing of a gene in the subject.
E6. The method of any one of E1-E5, wherein the siRNA includes (i) an antisense strand having complementarity to a portion of a gene encoding a positive regulator of a gene for which increased expression and/or activity (relative, e.g., to the level of expression and/or activity observed in a reference subject) is associated with a disease state.
E7. The method of any one of E1-E5, wherein the siRNA includes (i) an antisense strand having complementarity to a portion of a gene encoding a negative regulator of a gene for which decreased expression and/or activity (relative, e.g., to the level of expression and/or activity observed in a reference subject) is associated with a disease state.
E8. The method of any one of E1-E5, wherein the siRNA includes (i) an antisense strand having complementarity to a splice isoform of a gene for which overexpression of the splice isoform relative to the expression of the splice isoform in a reference subject is associated with a disease state.
E9. The method of any one of E6-E8, wherein the siRNA includes (ii) a sense strand having complementarity to the antisense strand.
E10. The method of any one of any one of E1-E9, wherein the silencing of the microglial gene in the subject treats a disease state in the subject.
E11. The method of any one of E1-E10, wherein the disease is a neuroinflammatory or neurodegenerative disease.
E12. The method of any one of E2-E10, wherein the dysregulated gene is selected from the group consisting of ABCA7, ABI3, ADAM10, APOC1, APOE, AXL, BIN1, C1QA, C3, C9ORF72, CASS4, CCL5, CD2AP, CD33, CD68, CLPTM1, CLU, CR1, CSF1, CST7, CTSB, CTSD, CTSL, CXCL10, CXCL13, DSG2, ECHDC3, EPHA1, FABP5, FERMT2, FTH1, GNAS, GRN, HBEGF, HLA-DRB1, HLA-DRB5, IFIT1, IFIT3, IFITM3, IFNAR1, IFNAR2, IGF1, IL10RA, ILIA, IL1B, IL1RAP, INPP5D, ITGAM, ITGAX, LILRB4, LPL, MEF2C, MMP12, MS4A4A, MS4A6A, NLRP3, NME8, NOS2, PICALM, PILRA, PLCG2, PTK2B, SCIMP, SLC24A4, SORL1, SPI1, SPP1, SPPL2A, TBK1, TNF, TREM2, TREML2, TYROBP, and ZCWPW1 or negative regulator, positive regulator, or splice isoform thereof.
E13. The method of any one of E1-E12, wherein the subject is a mammal, e.g., a human.
E14. The method of any one of E1-E13, wherein the branched siRNA is administered to the subject intrathecally, intracerebroventricularly, or intrastriatally.
E15. The method of any one of E1-E14, wherein the branched siRNA is administered to the subject intrathecally.
E16. The method of any one of E1-E14, wherein the branched siRNA is administered to the subject intracerebroventricularly.
E17. The method of any one of E1-E14, wherein the branched siRNA is administered to the subject intrastriatally.
E18. The method of any one of E1-17, wherein the siRNA molecule is di-branched.
E19. The method of any one of E1-17, wherein the siRNA molecule is tri-branched.
E20. The method of any one of E1-17, wherein the siRNA molecule is tetra-branched.
E21. The method of any one of E1-20, wherein the siRNA comprises (i) an antisense strand having complementarity to one or more of genes selected from the group consisting of APOE, BIN1, C1QA, C3, C9ORF72, CCL5, CD33, CLU/APOJ, CR1, CXCL10, CXCL13, IFIT1, IFIT3, IFITM3, IFNAR1, IFNAR2, IL10RA, ILIA, IL1B, IL1RAP, INPP5D, ITGAM, MEF2C, MMP12, NLRP3, NOS2, PILRA, PLCG2, PTK2B, SLC24A4, TBK1, and TNF and (ii) a sense strand having complementarity to the antisense strand.
E22. The method of E21, wherein the antisense strand has the following formula, in the 5′-to-3′ direction:

Z-((A-P-)ₙ(B-P-)ₘ)_{q};
wherein Z is a 5′ phosphorus stabilizing moiety;
each A is, independently, a 2′-O-methyl (2′-O-Me) ribonucleoside;
each B is, independently, a 2′-fluoro-ribonucleoside;
each P is, independently, an internucleoside linkage selected from a phosphodiester linkage and a phosphorothioate linkage;
n is an integer from 1 to 5;
m is an integer from 1 to 5; and
q is an integer between 1 and 15.
E23. The method of E22, wherein Z is represented in any one of Formula I-VIII:

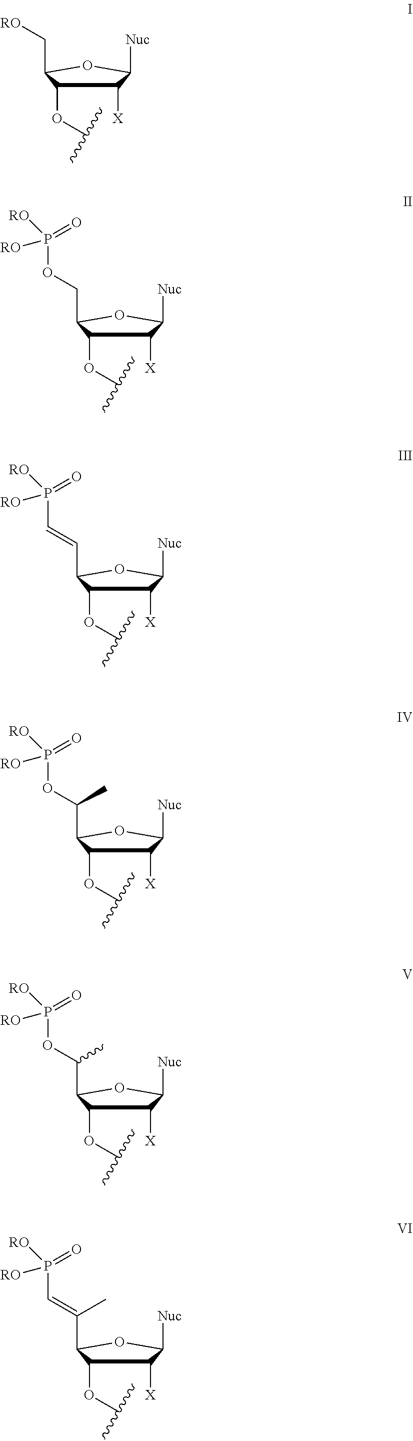

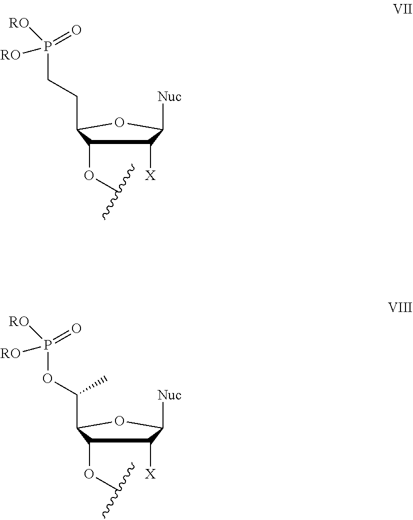

wherein Nuc represents a nucleobase, such as adenine, uracil, guanine, thymine, or cytosine, and R represents optionally substituted alkyl, optionally substituted alkenyl, or optionally substituted alkynyl (e.g., optionally substituted C1-C6 alkyl, optionally substituted C2-C6 alkenyl, or optionally substituted C2-C6 alkynyl), phenyl, benzyl, hydroxy, or hydrogen.
E24. The method of E22 or E23, wherein Z is (E)-vinylphosphonate represented in Formula III.
E25. The method of any one of E22-E24, wherein n is from 1 to 4.
E26. The method of any one of E22-E25, wherein n is from 1 to 3.
E27. The method of any one of E22-E26, wherein n is from 1 to 2.
E28. The method of any one of E22-E27, wherein n is 1.
E29. The method of any one of E22-E28, wherein m is from 1 to 4.
E30. The method of any one of E22-E29, wherein m is from 1 to 3.
E31. The method of any one of E22-E30, wherein m is from 1 to 2.
E32. The method of any one of E22-E31, wherein m is 1.
E33. The method of any one of E22-E32, wherein n and m are each 1.
E34. The method of any one of E22-E33, wherein 10% or less of the ribonucleosides are 2′-O-Me ribonucleoside.
E35. The method of any one of E22-E34, wherein at least 10% of the ribonucleosides are 2′-O-Me ribonucleoside.
E36. The method of any one of E22-E35, wherein at least 20% of the ribonucleosides are 2′-O-Me ribonucleoside.
E37. The method of any one of E22-E36, wherein at least 30% of the ribonucleosides are 2′-O-Me ribonucleoside.
E38. The method of any one of E22-E37, wherein at least 40% of the ribonucleosides are 2′-O-Me ribonucleoside.
E39. The method of any one of E22-E38, wherein at least 50% of the ribonucleosides are 2′-O-Me ribonucleoside.
E40. The method of any one of E22-E39, wherein at least 60% of the ribonucleosides are 2′-O-Me ribonucleoside.
E41. The method of any one of E22-E40, wherein at least 70% of the ribonucleosides are 2′-O-Me ribonucleoside.
E42. The method of any one of E22-E41, wherein at least 80% of the ribonucleosides are 2′-O-Me ribonucleoside.
E43. The method of any one of E22-E42, wherein at least 90% of the ribonucleosides are 2′-O-Me ribonucleoside.
E44. The method of any one of E22-E43, wherein 10% or less of the internucleoside linkages are phosphodiester linkages or phosphorothioate linkages.
E45. The method of any one of E22-E44, wherein at least 10% of the internucleoside linkages are phosphodiester linkages or phosphorothioate linkages.
E46. The method of any one of E22-E45, wherein at least 20% of the internucleoside linkages are phosphodiester linkages or phosphorothioate linkages.
E47. The method of any one of E22-E46, wherein at least 30% of the internucleoside linkages are phosphodiester linkages or phosphorothioate linkages.
E48. The method of any one of E22-E47, wherein at least 40% of the internucleoside linkages are phosphodiester linkages or phosphorothioate linkages.
E49. The method of any one of E22-E48, wherein at least 50% of the internucleoside linkages are phosphodiester linkages or phosphorothioate linkages.
E50. The method of any one of E22-E49, wherein at least 60% of the internucleoside linkages are phosphodiester linkages or phosphorothioate linkages.
E51. The method of any one of E22-E50, wherein at least 70% of the internucleoside linkages are phosphodiester linkages or phosphorothioate linkages.
E52. The method of any one of E22-E51, wherein at least 80% of the internucleoside linkages are phosphodiester linkages or phosphorothioate linkages.
E53. The method of any one of E22-E52, wherein at least 90% of the internucleoside linkages are phosphodiester linkages or phosphorothioate linkages.
E54. The method of any one of E22-E53, wherein 100% of the internucleoside linkages are phosphodiester linkages or phosphorothioate linkages.
E55. The method of any one of E22-E54, wherein 9 internucleoside linkages are phosphodiester linkages or phosphorothioate linkages.
E56. The method of any one of E22-E55, wherein the length of the antisense strand is between 10 and nucleotides.
E57. The method of any one of E22-E56, wherein the length of the antisense strand is between 15 and nucleotides.
E58. The method of any one of E22-E57, wherein the length of the antisense strand is between 18 and 23 nucleotides.
E59. The method of any one of E22-E58, wherein the length of the antisense strand is 18 nucleotides.
E60. The method of any one of E22-E56, wherein the length of the antisense strand is 19 nucleotides.
E61. The method of any one of E22-E56, wherein the length of the antisense strand is 20 nucleotides.
E62. The method of any one of E22-E56, wherein the length of the antisense strand is 21 nucleotides.
E63. The method of any one of E22-E56, wherein the length of the antisense strand is 22 nucleotides.
E64. The method of any one of E22-E56, wherein the length of the antisense strand is 23 nucleotides.
E65. The method of any one of E22-E56, wherein the length of the antisense strand is 24 nucleotides.
E66. The method of any one of E22-E56, wherein the length of the antisense strand is 25 nucleotides.
E67. The method of any one of E22-E56, wherein the length of the antisense strand is 26 nucleotides.
E68. The method of any one of E22-E56, wherein the length of the antisense strand is 27 nucleotides.
E69. The method of any one of E22-E56, wherein the length of the antisense strand is 28 nucleotides.
E70. The method of any one of E22-E56, wherein the length of the antisense strand is 29 nucleotides.
E71. The method of any one of E22-E56, wherein the length of the antisense strand is 30 nucleotides.
E72. The method of E22, wherein the antisense strand includes a structure of Formula A1, wherein Formula A1 is:

A-T-B-T-A-P-A-P-A-P-A-P-A-P-A-P-A-P-A-P-A-P-A-P-A-P-B-T-A-T-A-T-A-T-A-T-A-T-A-T Formula A1;

wherein A represents a 2′-O-methyl ribonucleoside, B represents a 2′-F ribonucleoside, T represents a phosphorothioate internucleoside linkage, and P represents a phosphodiester internucleoside linkage.
E73. The method of E22, wherein the antisense strand includes a structure of Formula A2, wherein Formula A2 is:

A-T-A-T-A-P-B-P-B-P-B-P-A-P-A-P-A-P-A-P-A-P-A-P-A-P-B-T-A-T-A-T-A-T-A-T-A-T-A-T (Formula A2);

wherein A represents a 2′-O-methyl ribonucleoside, B represents a 2′-F ribonucleoside, T represents a phosphorothioate internucleoside linkage, and P represents a phosphodiester internucleoside linkage.
E74. The method of E22, wherein the antisense strand includes a structure of Formula A3, wherein Formula A3 is:

A-T-B-T-A-P-B-P-B-P-B-P-A-P-A-P-A-P-A-P-A-P-A-P-A-P-B-T-A-T-A-T-A-T-A-T-A-T-A-T (Formula A3)

wherein A represents a 2′-O-methyl ribonucleoside, B represents a 2′-F ribonucleoside, T represents a phosphorothioate internucleoside linkage, and P represents a phosphodiester internucleoside linkage.
E75. The method of E22, wherein the antisense strand includes a structure of Formula A4, wherein Formula A4 is:

A-T-B-T-A-P-A-P-A-P-A-P-A-P-A-P-A-P-A-P-A-P-A-P-A-P-B-T-A-T-A-T-A-T-A-T-A-T-A-T (Formula A4)

wherein A represents a 2′-O-methyl ribonucleoside, B represents a 2′-F ribonucleoside, T represents a phosphorothioate internucleoside linkage, and P represents a phosphodiester internucleoside linkage.
E76. The method of E22, wherein the antisense strand includes a structure of Formula A5, wherein Formula A5 is:

A-T-B-T-A-P-A-P-A-P-B-P-A-P-A-P-A-P-A-P-A-P-A-P-A-P-B-T-A-T-B-T-A-T-A-T-A-T-A-T (Formula A5)

wherein A represents a 2′-O-methyl ribonucleoside, B represents a 2′-F ribonucleoside, T represents a phosphorothioate internucleoside linkage, and P represents a phosphodiester internucleoside linkage.
E77. The method of E22, wherein the sense strand has the following formula in the 5′-to-3′ direction:

Y-((A-P-)ₙ(B-P-)ₘ)_{q}L-((B-P-)ₘ(A-P-)ₙ)_{q};
wherein Y is a hydrophobic moiety (e.g., cholesterol, vitamin D, or tocopherol) moiety;
L is a linker;
each A is, independently, a 2′-O-Me ribonucleoside;
each B is, independently, a 2′-fluoro-ribonucleoside;
each P is, independently, an internucleoside linkage selected from a phosphodiester linkage and a phosphorothioate linkage;
n is an integer from 1 to 5;
m is an integer from 1 to 5; and
each q is an integer between 1 and 15.
E78. The method of E77, wherein Y is cholesterol.
E79. The method of E77, wherein Y is tocopherol.
E80. The method of any one of E77-E79, wherein L is an ethylene glycol oligomer.
E81. The method of any one of E77-E80, wherein L is tetraethylene glycol.
E82. The method of any one of E77-E81, wherein the linker attaches to the sense strand by way of a covalent bond-forming moiety.
E83. The method of E82, wherein the covalent bond-forming moiety is selected from the group consisting of an alkyl, ester, amide, carbamate, phosphonate, phosphate, phosphorothioate, phosphoroamidate, triazole, urea, and formacetal.
E84. The method of E77, wherein L includes a structure of Formula L1, wherein Formula L1 is:

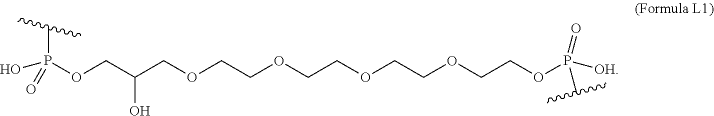

E85. The method of E77, wherein L includes a structure of Formula L2, wherein Formula L2 is:

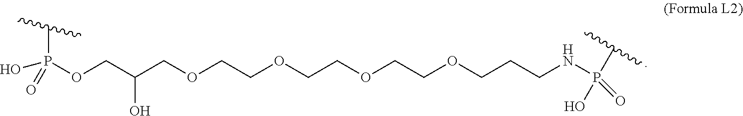

E86. The method of E77, wherein L includes a structure of Formula L3, wherein Formula L3 is:

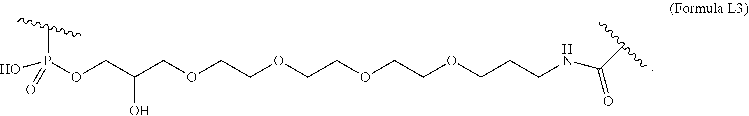

E87. The method of E77, wherein L includes a structure of Formula L4, wherein Formula L4 is:

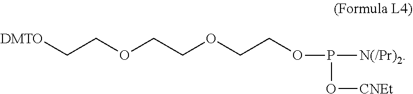

E88. The method of E77, wherein L includes a structure of Formula L5, wherein Formula L5 is:

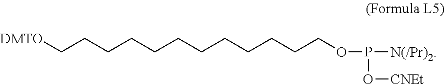

E89. The method of E77, wherein L includes a structure of Formula L6, wherein Formula L6 is:

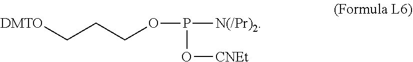

E90. The method of E77, wherein L includes a structure of Formula L7, wherein Formula L7 is:

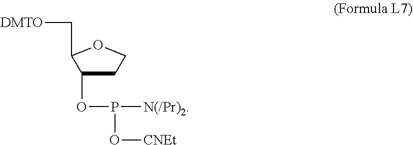

E91. The method of E77, wherein L includes a structure of Formula L8, wherein Formula L8 is:

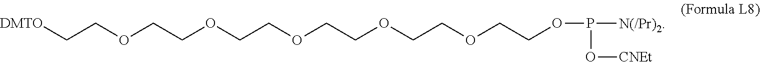

E92. The method of E77, wherein L includes a structure of Formula L9, wherein Formula L9 is:

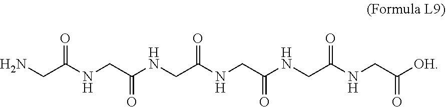 [SMILES: NCC(=O)NCC(=O)NCC(=O)NCC(=O)NCC(=O)NCC(=O)O]

E93. The method of any one of E77-E92, wherein each P is independently selected from a phosphodiester linkage and a phosphorothioate linkage.
E94. The method of any one of E77-E93, wherein n is from 1 to 4.
E95. The method of any one of E77-E94, wherein n is from 1 to 3.
E96. The method of any one of E77-E95, wherein n is from 1 to 2.
E97. The method of any one of E77-E96, wherein n is 1.
E98. The method of any one of E77-E97, wherein m is from 1 to 4.
E99. The method of any one of E77-E98, wherein m is from 1 to 3.
E100. The method of any one of E77-E99, wherein m is from 1 to 2.
E101. The method of any one of E77-E100, wherein m is 1.
E102. The method of any one of E77-E101, wherein n and m are each 1.
E103. The method of any one of E77-E102, wherein 10% or less of the ribonucleosides are 2′-O-Me ribonucleoside.
E104. The method of any one of E77-E103, wherein at least 10% of the ribonucleosides are 2′-O-Me ribonucleoside.
E105. The method of any one of E77-E104, wherein at least 20% of the ribonucleosides are 2′-O-Me ribonucleoside.
E106. The method of any one of E77-E105, wherein at least 30% of the ribonucleosides are 2′-O-Me ribonucleoside.
E107. The method of any one of E77-E106, wherein at least 40% of the ribonucleosides are 2′-O-Me ribonucleoside.
E108. The method of any one of E77-E107, wherein at least 50% of the ribonucleosides are 2′-O-Me ribonucleoside.
E109. The method of any one of E77-E108, wherein at least 60% of the ribonucleosides are 2′-O-Me ribonucleoside.
E110. The method of any one of E77-E109, wherein at least 70% of the ribonucleosides are 2′-O-Me ribonucleoside.
E111. The method of any one of E77-E110, wherein at least 80% of the ribonucleosides are 2′-O-Me ribonucleoside.
E112. The method of any one of E77-E111, wherein at least 90% of the ribonucleosides are 2′-O-Me ribonucleoside.
E113. The method of any one of E77-E112, wherein 10% or less of the internucleoside linkages are phosphodiester linkages or phosphorothioate.
E114. The method of any one of E77-E113, wherein at least 10% of the internucleoside linkages are phosphodiester linkages or phosphorothioate.
E115. The method of any one of E77-E114, wherein at least 20% of the internucleoside linkages are phosphodiester linkages or phosphorothioate.
E116. The method of any one of E77-E115, wherein at least 30% of the internucleoside linkages are phosphodiester linkages or phosphorothioate.
E117. The method of any one of E77-E116, wherein at least 40% of the internucleoside linkages are phosphodiester linkages or phosphorothioate.
E118. The method of any one of E77-E117, wherein at least 50% of the internucleoside linkages are phosphodiester linkages or phosphorothioate.
E119. The method of any one of E77-E118, wherein at least 60% of the internucleoside linkages are phosphodiester linkages or phosphorothioate.
E120. The method of any one of E77-E119, wherein at least 70% of the internucleoside linkages are phosphodiester linkages or phosphorothioate.
E121. The method of any one of E77-E120, wherein at least 80% of the internucleoside linkages are phosphodiester linkages or phosphorothioate.
E122. The method of any one of E77-E121, wherein at least 90% of the internucleoside linkages are phosphodiester linkages or phosphorothioate.
E123. The method of any one of E77-E122, wherein 100% of the internucleoside linkages are phosphodiester linkages or phosphorothioate.
E124. The method of any one of E77-E123, wherein the length of the sense strand is between 12 and 30 nucleotides.
E125. The method of any one of E77-E124, wherein the length of the sense strand is between 14 and 28 nucleotides.
E126. The method of any one of E77-E125, wherein the length of the sense strand is between 16 and 26 nucleotides.
E127. The method of any one of E77-E126, wherein the length of the sense strand is between 18 and 24 nucleotides.
E128. The method of any one of E77-E125, wherein the length of the sense strand is 14 nucleotides.
E129. The method of any one of E77-E125, wherein the length of the sense strand is 15 nucleotides.
E130. The method of any one of E77-E125, wherein the length of the sense strand is 16 nucleotides.
E131. The method of any one of E77-E125, wherein the length of the sense strand is 17 nucleotides.
E132. The method of any one of E77-E125, wherein the length of the sense strand is 18 nucleotides.
E133. The method of any one of E77-E125, wherein the length of the sense strand is 19 nucleotides.
E134. The method of any one of E77-E125, wherein the length of the sense strand is 20 nucleotides.
E135. The method of any one of E77-E125, wherein the length of the sense strand is 21 nucleotides.
E136. The method of any one of E77-E125, wherein the length of the sense strand is 22 nucleotides.
E137. The method of any one of E77-E125, wherein the length of the sense strand is 23 nucleotides.
E138. The method of any one of E77-E125, wherein the length of the sense strand is 24 nucleotides.
E139. The method of any one of E77-E125, wherein the length of the sense strand is 25 nucleotides.
E140. The method of any one of E77-E125, wherein the length of the sense strand is 26 nucleotides.
E141. The method of any one of E77-E125, wherein the length of the sense strand is 27 nucleotides.
E142. The method of any one of E77-E125, wherein the length of the sense strand is 28 nucleotides.
E143. The method of any one of E77-E125, wherein the length of the sense strand is 29 nucleotides.
E144. The method of any one of E77-E125, wherein the length of the sense strand is 30 nucleotides.
E145. The method of any one of E77-E144, wherein 4 internucleoside linkages are phosphorothioate linkages.
E146. The method of E77, wherein the sense strand includes a structure of Formula S1, wherein Formula S1 is:

A-T-A-T-A-P-A-P-A-P-A-P-A-P-A-P-A-P-A-P-A-P-A-P-A-P-A-T-A-T Formula S1;

wherein A represents a 2′-O-methyl ribonucleoside, B represents a 2′-F ribonucleoside, T represents a phosphorothioate internucleoside linkage, and P represents a phosphodiester internucleoside linkage.
E147. The method of E77, wherein the sense strand includes a structure of Formula S2, wherein Formula S2 is:

A-T-A-T-A-P-A-P-A-P-A-P-B-P-A-P-A-P-B-P-B-P-A-P-A-P-A-T-A-T Formula S2;

wherein A represents a 2′-O-methyl ribonucleoside, B represents a 2′-F ribonucleoside, T represents a phosphorothioate internucleoside linkage, and P represents a phosphodiester internucleoside linkage.
E148. The method of E77, wherein the sense strand includes a structure of Formula S3, wherein Formula S3 is:

A-T-A-T-A-P-A-P-A-P-A-P-B-P-A-P-B-P-B-P-B-P-A-P-A-P-A-T-A-T Formula S3;

wherein A represents a 2′-O-methyl ribonucleoside, B represents a 2′-F ribonucleoside, T represents a phosphorothioate internucleoside linkage, and P represents a phosphodiester internucleoside linkage.
E149. A branched siRNA molecule comprising a sense strand and an antisense strand, wherein the antisense strand comprises a region having complementarity to a segment of contiguous nucleotides within a gene selected from the group consisting of APOE, BIN1, C1QA, C3, C9ORF72, CCL5, CD33, CLU/APOJ, CR1, CXCL10, CXCL13, IFIT1, IFIT3, IFITM3, IFNAR1, IFNAR2, IL10RA, ILIA, IL1B, IL1RAP, INPP5D, ITGAM, MEF2C, MMP12, NLRP3, NOS2, PILRA, PLCG2, PTK2B, SLC24A4, TBK1, and TNF or a negative regulator, positive regulator, or splice isoform thereof.
E150. The molecule of E149, wherein the siRNA molecule is di-branched.
E151. The molecule of E149, wherein the siRNA molecule is tri-branched.
E152. The molecule of any one of E149, wherein the siRNA molecule is tetra-branched.
E153. The molecule of any one of E149-E152, wherein the antisense strand of the branched siRNA has the following formula in the 5′-to-3′ direction:

Z-((A-P-)ₙ(B-P-)ₘ)_{q};
wherein Z is a 5′ phosphorus stabilizing moiety;
each A is, independently, a 2′-O-Me ribonucleoside;
each B is, independently, a 2′-fluoro-ribonucleoside;
each P is, independently, an internucleoside linkage selected from a phosphodiester linkage and a phosphorothioate linkage;
n is an integer from 1 to 5; m is an integer from 1 to 5; and
q is an integer between 1 and 15.
E154. The molecule of E153, wherein Z is represented in any one of Formula I-VIII:

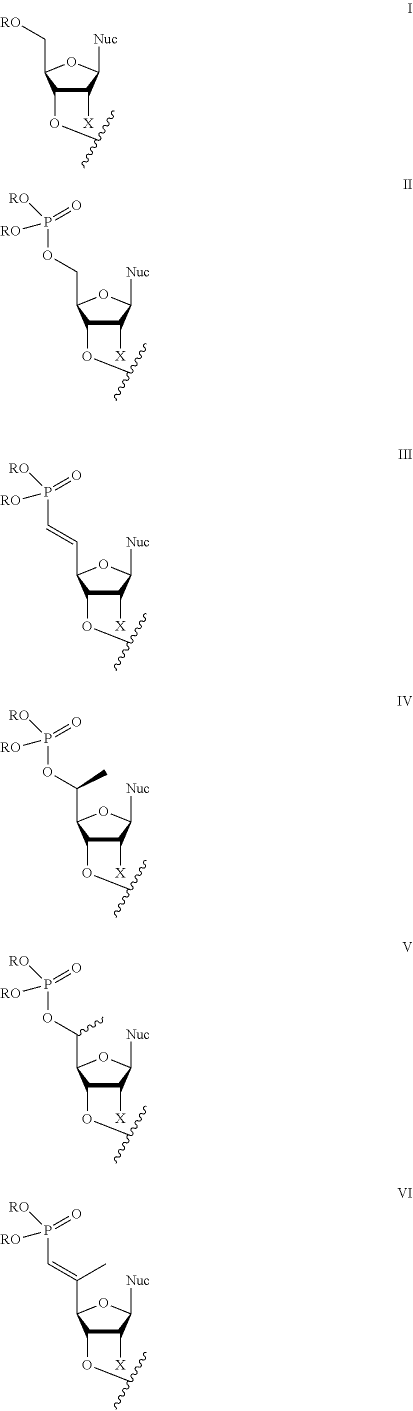

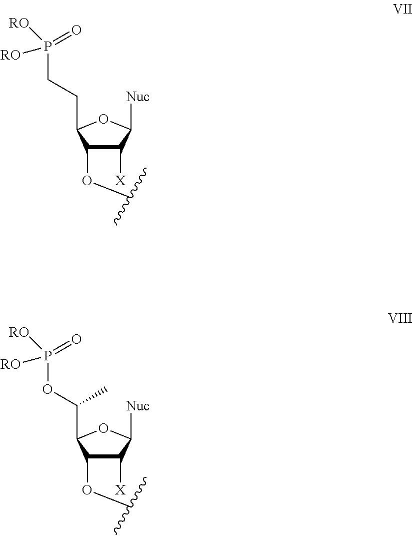

wherein Nuc represents a nucleobase, such as adenine, uracil, guanine, thymine, or cytosine, and R represents optionally substituted alkyl, optionally substituted alkenyl, or optionally substituted alkynyl (e.g., optionally substituted C1-C6 alkyl, optionally substituted C2-C6 alkenyl, or optionally substituted C2-C6 alkynyl), phenyl, benzyl, hydroxy, or hydrogen.
E155. The molecule of E153 or E154, wherein Z is (E)-vinylphosphonate as represented in Formula III.
E156. The molecule of any one of E153-E99, wherein each P is independently selected from phosphodiester and phosphorothioate.
E157. The molecule of any one of E153-E156, wherein n is from 1 to 4.
E158. The molecule of any one of E153-E157, wherein n is from 1 to 3.
E159. The molecule of any one of E153-E158, wherein n is from 1 to 2.
E160. The molecule of any one of E153-E159, wherein n is 1.
E161. The molecule of any one of E153-E160, wherein m is from 1 to 4.
E162. The molecule of any one of E153-E161, wherein m is from 1 to 3.
E163. The molecule of any one of E153-E162, wherein m is from 1 to 2.
E164. The molecule of any one of E153-E163, wherein m is 1.
E165. The molecule of any one of E153-E164, wherein n and m are each 1.
E166. The molecule of any one of E153-E165, wherein 10% or less of the ribonucleosides are 2′-O-Me ribonucleoside.
E167. The molecule of any one of E153-E166, wherein at least 10% of the ribonucleosides are 2′-O-Me ribonucleoside.
E168. The molecule of any one of E153-E167, wherein at least 20% of the ribonucleosides are 2′-O-Me ribonucleoside.
E169. The molecule of any one of E153-E168, wherein at least 30% of the ribonucleosides are 2′-O-Me ribonucleoside.
E170. The molecule of any one of E153-E169, wherein at least 40% of the ribonucleosides are 2′-O-Me ribonucleoside.
E171. The molecule of any one of E153-E170, wherein at least 50% of the ribonucleosides are 2′-O-Me ribonucleoside.
E172. The molecule of any one of E153-E171, wherein at least 60% of the ribonucleosides are 2′-O-Me ribonucleoside.
E173. The molecule of any one of E153-E172, wherein at least 70% of the ribonucleosides are 2′-O-Me ribonucleoside.
E174. The molecule of any one of E153-E173, wherein at least 80% of the ribonucleosides are 2′-O-Me ribonucleoside.
E175. The molecule of any one of E153-E174, wherein at least 90% of the ribonucleosides are 2′-O-Me ribonucleoside.
E176. The molecule of any one of E153-E175, wherein 10% or less of the internucleoside linkages are phosphodiester linkages or phosphorothioate.
E177. The molecule of any one of E153-E176, wherein at least 10% of the internucleoside linkages are phosphodiester linkages or phosphorothioate.
E178. The molecule of any one of E153-E177, wherein at least 20% of the internucleoside linkages are phosphodiester linkages or phosphorothioate.
E179. The molecule of any one of E153-E178, wherein at least 30% of the internucleoside linkages are phosphodiester linkages or phosphorothioate.
E180. The molecule of any one of E153-E179, wherein at least 40% of the internucleoside linkages are phosphodiester linkages or phosphorothioate.
E181. The molecule of any one of E153-E180, wherein at least 50% of the internucleoside linkages are phosphodiester linkages or phosphorothioate.
E182. The molecule of any one of E153-E181, wherein at least 60% of the internucleoside linkages are phosphodiester linkages or phosphorothioate.
E183. The molecule of any one of E153-E182, wherein at least 70% of the internucleoside linkages are phosphodiester linkages or phosphorothioate.
E184. The molecule of any one of E153-E183, wherein at least 80% of the internucleoside linkages are phosphodiester linkages or phosphorothioate.
E185. The molecule of any one of E153-E184, wherein at least 90% of the internucleoside linkages are phosphodiester linkages or phosphorothioate.
E186. The molecule of any one of E153-E185, wherein 100% of the internucleoside linkages are phosphodiester linkages or phosphorothioate.
E187. The molecule of any one of E153-E186, wherein the length of the antisense strand is between 10 and 30 nucleotides.
E188. The molecule of any one of E153-E187, wherein the length of the antisense strand is between 15 and 25 nucleotides.
E189. The molecule of any one of E153-E188, wherein the length of the antisense strand is between 18 and 23 nucleotides.
E190. The molecule of any one of E153-E187, wherein the length of the antisense strand is 18 nucleotides.
E191. The molecule of any one of E153-E187, wherein the length of the antisense strand is 19 nucleotides.
E192. The molecule of any one of E153-E187, wherein the length of the antisense strand is 20 nucleotides.
E193. The molecule of any one of E153-E187, wherein the length of the antisense strand is 21 nucleotides.
E194. The molecule of any one of E153-E187, wherein the length of the antisense strand is 22 nucleotides.
E195. The molecule of any one of E153-E187, wherein the length of the antisense strand is 23 nucleotides.
E196. The molecule of any one of E153-E187, wherein the length of the antisense strand is 24 nucleotides.
E197. The molecule of any one of E153-E187, wherein the length of the antisense strand is 25 nucleotides.
E198. The molecule of any one of E153-E187, wherein the length of the antisense strand is 26 nucleotides.
E199. The molecule of any one of E153-E187, wherein the length of the antisense strand is 27 nucleotides.
E200. The molecule of any one of E153-E187, wherein the length of the antisense strand is 28 nucleotides.
E201. The molecule of any one of E153-E187, wherein the length of the antisense strand is 29 nucleotides.
E202. The molecule of any one of E153-E187, wherein the length of the antisense strand is 30 nucleotides.
E203. The molecule of any one of E149-E202, wherein 9 internucleoside linkages are phosphorothioate.
E204. The molecule of any one of E149-E203, wherein the sense strand of the branched siRNA has the following formula in the 5′-to-3′ direction:

Y-((A-P-)ₙ(B-P-)ₘ)_{q}L-((B-P-)ₘ(A-P-)ₙ)_{q};
wherein Y is a hydrophobic moiety (e.g., cholesterol, vitamin D, or tocopherol);
L is a linker;
each A is, independently, a 2′-O-Me ribonucleoside;
each B is, independently, a 2′-fluoro-ribonucleoside;
each P is, independently, an internucleoside linkage selected from a phosphodiester linkage and a
phosphorothioate linkage;
n is an integer from 1 to 5;
m is an integer from 1 to 5; and
q is an integer between 1 and 15.
E205. The molecule of E204, wherein Y is cholesterol.
E206. The molecule of E204, wherein Y is tocopherol.
E207. The molecule of any one of E204-E206, wherein L is an ethylene glycol oligomer.
E208. The molecule of E207, wherein L is tetraethylene glycol.
E209. The molecule of any one of E204-E208, wherein L attaches to the sense strand by way of a covalent bond-forming moiety.
E210. The molecule of E209, wherein the covalent bond-forming moiety is selected from the group consisting of an alkyl, ester, amide, carbamate, phosphonate, phosphate, phosphorothioate, phosphoroamidate, triazole, urea, and formacetal.
E211. The molecule of E204, wherein L includes a structure of Formula L1, wherein Formula L1 is:

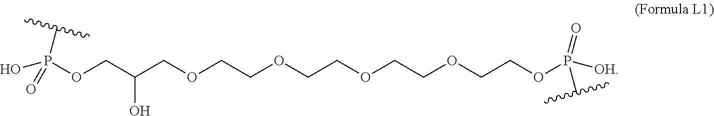 [SMILES: *P(=O)(O)OCCOCCOCCOCCOCC(O)COP(*)(=O)O]

E212. The molecule of E204, wherein L includes a structure of Formula L2, wherein Formula L2 is:

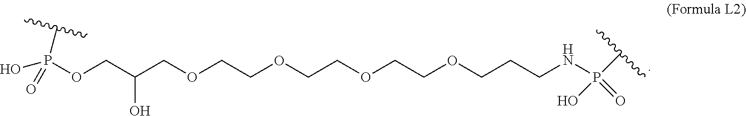 [SMILES: *P(=O)(O)NCCCOCCOCCOCCOCC(O)COP(*)(=O)O]

E213. The molecule of E204, wherein L includes a structure of Formula L3, wherein Formula L3 is:

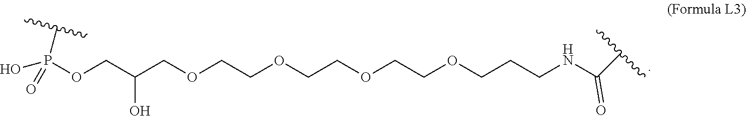 [SMILES: *C(=O)NCCCOCCOCCOCCOCC(O)COP(*)(=O)O]

E214. The molecule of E204, wherein L includes a structure of Formula L4, wherein Formula L4 is:

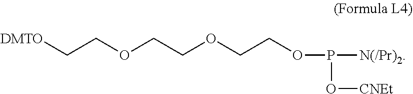

E215. The molecule of E204, wherein L includes a structure of Formula L5, wherein Formula L5 is:

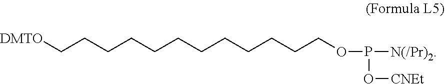

E216. The molecule of E204, wherein L includes a structure of Formula L6, wherein Formula L6 is:

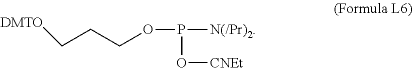

E217. The molecule of E204, wherein L includes a structure of Formula L7, wherein Formula L7 is:

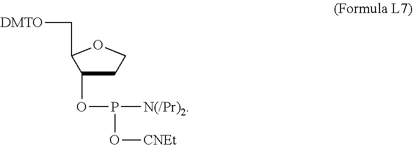

E218. The molecule of E204, wherein L includes a structure of Formula L8, wherein Formula L8 is:

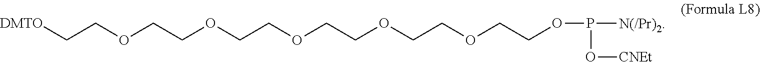

E219. The molecule of E204, wherein L includes a structure of Formula L9, wherein Formula L9 is:

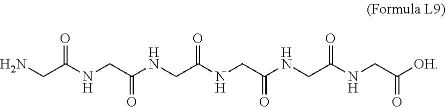 [SMILES: NCC(=O)NCC(=O)NCC(=O)NCC(=O)NCC(=O)NCC(=O)O]

E220. The molecule of any one of E204-E210, wherein each P is independently selected from phosphodiester and phosphorothioate.
E221. The molecule of any one of E204-E141, wherein n is from 1 to 4.
E222. The molecule of any one of E204-E142, wherein n is from 1 to 3.
E223. The molecule of any one of E204-E143, wherein n is from 1 to 2.
E224. The molecule of any one of E204-E144, wherein n is 1.
E225. The molecule of any one of E204-E145, wherein m is from 1 to 4.
E226. The molecule of any one of E204-E146, wherein m is from 1 to 3.
E227. The molecule of any one of E204-E147, wherein m is from 1 to 2.
E228. The molecule of any one of E204-E148, wherein m is 1.
E229. The molecule of any one of E204-E149, wherein n and m are each 1.
E230. The molecule of any one of E204-E150, wherein 10% or less of the ribonucleosides are 2′-O-Me ribonucleoside.
E231. The molecule of any one of E204-E151, wherein at least 10% of the ribonucleosides are 2′-O-Me ribonucleoside.
E232. The molecule of any one of E204-E152, wherein at least 20% of the ribonucleosides are 2′-O-Me ribonucleoside.
E233. The molecule of any one of E204-E153, wherein at least 30% of the ribonucleosides are 2′-O-Me ribonucleoside.
E234. The molecule of any one of E204-E154, wherein at least 40% of the ribonucleosides are 2′-O-Me ribonucleoside.
E235. The molecule of any one of E204-E155, wherein at least 50% of the ribonucleosides are 2′-O-Me ribonucleoside.
E236. The molecule of any one of E204-E156, wherein at least 60% of the ribonucleosides are 2′-O-Me ribonucleoside.
E237. The molecule of any one of E204-E157, wherein at least 70% of the ribonucleosides are 2′-O-Me ribonucleoside.
E238. The molecule of any one of E204-E158, wherein at least 80% of the ribonucleosides are 2′-O-Me ribonucleoside.
E239. The molecule of any one of E204-E159, wherein at least 90% of the ribonucleosides are 2′-O-Me ribonucleoside.
E240. The molecule of any one of E204-E160, wherein 10% or less of the internucleoside linkages are phosphodiester linkages or phosphorothioate linkages.
E241. The molecule of any one of E204-E161, wherein at least 10% of the internucleoside linkages are phosphodiester linkages or phosphorothioate linkages.
E242. The molecule of any one of E204-E162, wherein at least 20% of the internucleoside linkages are phosphodiester linkages or phosphorothioate linkages.
E243. The molecule of any one of E204-E163, wherein at least 30% of the internucleoside linkages are phosphodiester linkages or phosphorothioate linkages.
E244. The molecule of any one of E204-E164, wherein at least 40% of the internucleoside linkages are phosphodiester linkages or phosphorothioate linkages.
E245. The molecule of any one of E204-E165, wherein at least 50% of the internucleoside linkages are phosphodiester linkages or phosphorothioate linkages.
E246. The molecule of any one of E204-E166, wherein at least 60% of the internucleoside linkages are phosphodiester linkages or phosphorothioate linkages.
E247. The molecule of any one of E204-E167, wherein at least 70% of the internucleoside linkages are phosphodiester linkages or phosphorothioate linkages.
E248. The molecule of any one of E204-E168, wherein at least 80% of the internucleoside linkages are phosphodiester linkages or phosphorothioate linkages.
E249. The molecule of any one of E204-E169, wherein at least 90% of the internucleoside linkages are phosphodiester linkages or phosphorothioate linkages.
E250. The molecule of any one of E204-E170, wherein 100% of the internucleoside linkages are phosphodiester linkages or phosphorothioate linkages.
E251. The molecule of any one of E204-E250, wherein the length of the sense strand is between 12 and nucleotides.
E252. The molecule of any one of E204-E251, wherein the length of the sense strand is between 14 and 28 nucleotides.
E253. The molecule of any one of E204-E252, wherein the length of the sense strand is between 16 and 26 nucleotides.
E254. The molecule of any one of E204-E253, wherein the length of the sense strand is between 18 and 24 nucleotides.
E255. The molecule of any one of E204-E251, wherein the length of the sense strand is 14 nucleotides.
E256. The molecule of any one of E204-E251, wherein the length of the sense strand is 15 nucleotides.
E257. The molecule of any one of E204-E251, wherein the length of the sense strand is 16 nucleotides.
E258. The molecule of any one of E204-E251, wherein the length of the sense strand is 17 nucleotides.
E259. The molecule of any one of E204-E251, wherein the length of the sense strand is 18 nucleotides.
E260. The molecule of any one of E204-E251, wherein the length of the sense strand is 19 nucleotides.
E261. The molecule of any one of E204-E251, wherein the length of the sense strand is 20 nucleotides.
E262. The molecule of any one of E204-E251, wherein the length of the sense strand is 21 nucleotides.
E263. The molecule of any one of E204-E251, wherein the length of the sense strand is 22 nucleotides.
E264. The molecule of any one of E204-E251, wherein the length of the sense strand is 23 nucleotides.
E265. The molecule of any one of E204-E251, wherein the length of the sense strand is 24 nucleotides.
E266. The molecule of any one of E204-E251, wherein the length of the sense strand is 25 nucleotides.
E267. The molecule of any one of E204-E251, wherein the length of the sense strand is 26 nucleotides.
E268. The molecule of any one of E204-E251, wherein the length of the sense strand is 27 nucleotides.
E269. The molecule of any one of E204-E251, wherein the length of the sense strand is 28 nucleotides.
E270. The molecule of any one of E204-E251, wherein the length of the sense strand is 29 nucleotides.
E271. The molecule of any one of E204-E251, wherein the length of the sense strand is 30 nucleotides.
E272. The molecule of any one of E204-E271, wherein 4 internucleoside linkages are phosphorothioate.
E273. A method of treating a subject diagnosed as having a disease associated with expression of a dysregulated microglial gene, the method comprising administering to the subject the branched siRNA molecule of any one of E149-E271.
E274. The method of any one of E11-E148 or E273, wherein the disease is a neuroinflammatory disease.
E275. The method of any one of E11-E148, E273, or E274, wherein the disease is a neurodegenerative disease.
E276. The method of any one of E11-E148 or E273-E275, wherein the disease is Alzheimer's disease.
E277. The method of any one of E11-E148 or E273-E275, wherein the disease is Amyotrophic Lateral

Sclerosis.
E278. The method of any one of E11-E148 or E273-E275, wherein the disease is Parkinson's disease.
E279. The method of any one of E11-E148 or E273-E275, wherein the disease is frontotemporal dementia.
E280. The method of any one of E11-E148 or E273-E275, wherein the disease is Huntington's disease.
E281. The method of any one of E11-E148 or E273-E275, wherein the disease is multiple sclerosis.
E282. The method of any one of E11-E148 or E273-E275, wherein the disease is progressive supranuclear palsy.
E283. The method of any one of E273, wherein the dysregulated microglial gene is selected from the group consisting of ABCA7, ABI3, ADAM10, APOC1, APOE, AXL, BIN1, C1QA, C3, C9ORF72, CASS4, CCL5, CD2AP, CD33, CD68, CLPTM1, CLU, CR1, CSF1, CST7, CTSB, CTSD, CTSL, CXCL10, CXCL13, DSG2, ECHDC3, EPHA1, FABP5, FERMT2, FTH1, GNAS, GRN, HBEGF, HLA-DRB1, HLA-DRB5, IFIT1, IFIT3, IFITM3, IFNAR1, IFNAR2, IGF1, IL10RA, ILIA, IL1B, IL1 RAP, INPP5D, ITGAM, ITGAX, LILRB4, LPL, MEF2C, MMP12, MS4A4A, MS4A6A, NLRP3, NME8, NOS2, PICALM, PILRA, PLCG2, PTK2B, SCIMP, SLC24A4, SORL1, SPI1, SPP1, SPPL2A, TBK1, TNF, TREM2, TREML2, TYROBP, and ZCWPW1.
E284. The method of E273, wherein the administering of the branched siRNA molecule to the subject results is silencing of a microglial gene in the subject.
E285. The method of E284, wherein silencing of a microglial gene comprises silencing of any one of the genes selected from group consisting of APOE, BIN1, C1QA, C3, C9ORF72, CCL5, CD33, CLU/APOJ, CR1, CXCL10, CXCL13, IFIT1, IFIT3, IFITM3, IFNAR1, IFNAR2, IL10RA, ILIA, IL1B, IL1RAP, INPP5D, ITGAM, MEF2C, MMP12, NLRP3, NOS2, PILRA, PLCG2, PTK2B, SLC24A4, TBK1, and TNF.
E286. The method of E273, wherein the microglial gene is an overactive disease driver gene (e.g., a dysregulated microglial gene).
E287. The method of E273, wherein the gene is a positive regulator of a gene for which increased expression and/or activity relative to the level of expression and/or activity observed in a reference subject is associated with a disease state.
E288. The method of E273, wherein the gene is a negative regulator of a gene for which decreased expression and/or activity relative to the level of expression and/or activity observed in a reference subject is associated with a disease state.
E289. The method of E273, wherein the gene is a splice isoform of a gene for which overexpression of the splice isoform relative to the expression of the splice isoform in a reference subject is associated with a disease state.
E290. The method of any one of E273-E289, wherein the subject is a human.

### Other Embodiments

Various modifications and variations of the described disclosure will be apparent to those skilled in the art without departing from the scope and spirit of the disclosure. Although the disclosure has been described in connection with specific embodiments, it should be understood that the disclosure as claimed should not be unduly limited to such specific embodiments. Indeed, various modifications of the described modes for carrying out the disclosure that are obvious to those skilled in the art are intended to be within the scope of the disclosure.

Other embodiments are in the claims.

1. A method of delivering a branched small interfering RNA (siRNA) molecule to a microglial cell in a subject in need of microglial gene silencing, the method comprising administering the branched siRNA molecule to the central nervous system of the subject.

2. The method of claim 1, wherein the subject has been diagnosed as having a disease associated with expression of a dysregulated microglial gene or dysregulated microglial gene pathway.

3. The method of claim 2, wherein the dysregulated microglial gene exhibits increased expression and/or activity in microglial cells of the subject as compared to the expression and/or activity of the microglial gene in microglial cells of a reference subject.

4. The method of claim 2, wherein the dysregulated microglial gene exhibits reduced expression and/or activity in microglial cells of the subject as compared to the expression and/or activity of the microglial gene in microglial cells of a reference subject.

5. The method of claim 1, wherein the microglial gene is a positive regulator of a gene for which increased expression and/or activity relative to the level of expression and/or activity observed in a reference subject is associated with a disease state.

6. The method of claim 1, wherein the microglial gene is a negative regulator of a gene for which decreased expression and/or activity relative to the level of expression and/or activity observed in a reference subject is associated with a disease state.

7. The method of claim 1, wherein the microglial gene is a splice isoform of a gene for which overexpression of the splice isoform relative to the expression of the splice isoform in a reference subject is associated with a disease state.

8. The method of any one of claims 2-7, wherein the disease is a neuroinflammatory or neurodegenerative disease.

9. The method of any one of claims 1-8, wherein the dysregulated gene is selected from the group consisting of ABCA7, ABI3, ADAM10, APOC1, APOE, AXL, BIN1, C1QA, C3, C9ORF72, CASS4, CCL5, CD2AP, CD33, CD68, CLPTM1, CLU, CR1, CSF1, CST7, CTSB, CTSD, CTSL, CXCL10, CXCL13, DSG2, ECHDC3, EPHA1, FABP5, FERMT2, FTH1, GNAS, GRN, HBEGF, HLA-DRB1, HLA-DRB5, IFIT1, IFIT3, IFITM3, IFNAR1, IFNAR2, IGF1, IL10RA, IL1A, IL1B, IL1RAP, INPP5D, ITGAM, ITGAX, LILRB4, LPL, MEF2C, MMP12, MS4A4A, MS4A6A, NLRP3, NME8, NOS2, PICALM, PILRA, PLCG2, PTK2B, SCIMP, SLC24A4, SORL1, SPI1, SPP1, SPPL2A, TBK1, TNF, TREM2, TREML2, TYROBP, and ZCVVPW1.

10. The method of any one of claims 1-9, wherein the subject is a human.

11. The method of any one of claims 1-10, wherein the branched siRNA is administered to the subject intrathecally, intracerebroventricularly, or intrastriatally.

12. The method of any one of claims 1-11, wherein the siRNA molecule is di-branched.

13. The method of any one of claims 1-12, wherein the siRNA comprises (i) an antisense strand having complementarity to one or more of genes selected from the group consisting of APOE, BIN1, C1QA, C3, C9ORF72, CCL5, CD33, CLU/APOJ, CR1, CXCL10, CXCL13, IFIT1, IFIT3, IFITM3, IFNAR1, IFNAR2, IL10RA, ILIA, IL1B, IL1RAP, INPP5D, ITGAM, MEF2C, MMP12, NLRP3, NOS2, PILRA, PLCG2, PTK2B, SLC24A4, TBK1, and TNF, and (ii) a sense strand having complementarity to the antisense strand.

14. The method of claim 13, wherein the antisense strand has the following formula, in the 5′-to-3′ direction:
Z-((A-P-)ₙ(B-P-)ₘ)_{q};
wherein Z is a 5′ phosphorus stabilizing moiety;
each A is, independently, a 2′-O-methyl (2′-O-Me) ribonucleoside;
each B is, independently, a 2′-fluoro (2′-F) ribonucleoside;
each P is, independently, an internucleoside linkage selected from a phosphodiester linkage and a phosphorothioate linkage;
n is an integer from 1 to 5;
m is an integer from 1 to 5; and q is an integer between 1 and 15

15. The method of claim 14, wherein Z is represented in any one of Formula I-VIII: 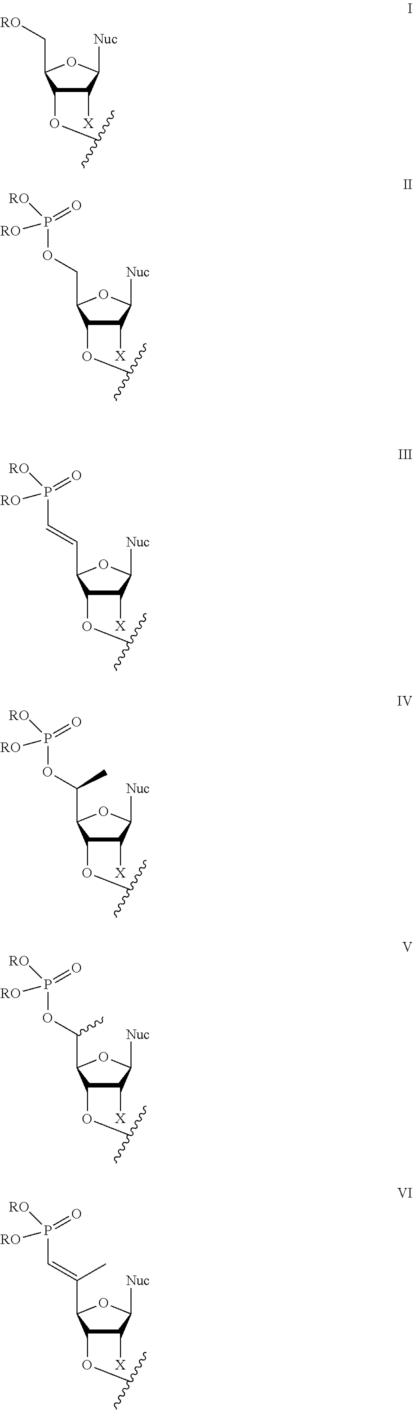 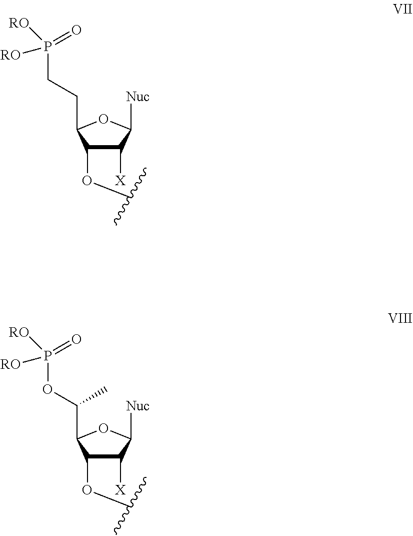
wherein Nuc represents a nucleobase selected from the group consisting of adenine, uracil, guanine, thymine, and cytosine, and R represents optionally substituted alkyl, optionally substituted alkenyl, optionally substituted alkynyl, phenyl, benzyl, hydroxy, or hydrogen.

16. The method of claim 14 or 15, wherein Z is (E)-vinylphosphonate represented in Formula III.

17. The method of any one of claims 13-16, wherein at least 50% of the ribonucleosides are 2′-O-Me ribonucleoside.

18. The method of any one of claims 13-17, wherein at least 60% of the ribonucleosides are 2′-O-Me ribonucleoside.

19. The method of any one of claims 13-18, wherein at least 70% of the ribonucleosides are 2′-O-Me ribonucleoside.

20. The method of any one of claims 13-19, wherein at least 80% of the ribonucleosides are 2′-O-Me ribonucleoside.

21. The method of any one of claims 13-20, wherein at least 90% of the ribonucleosides are 2′-O-Me ribonucleoside.

22. The method of any one of claims 13-21, wherein the length of the antisense strand is between 10 and 30 nucleotides.

23. The method of any one of claims 13-22, wherein the length of the antisense strand is between 15 and 25 nucleotides.

24. The method of claim 23, wherein the length of the antisense strand is 20 nucleotides.

25. The method of claim 23, wherein the length of the antisense strand is 21 nucleotides.

26. The method of claim 23, wherein the length of the antisense strand is 22 nucleotides.

27. The method of claim 23, wherein the length of the antisense strand is 23 nucleotides.

28. The method of claim 23, wherein the length of the antisense strand is 24 nucleotides.

29. The method of claim 23, wherein the length of the antisense strand is 25 nucleotides.

30. The method of claim 22, wherein the length of the antisense strand is 26 nucleotides.

31. The method of claim 22, wherein the length of the antisense strand is 27 nucleotides.

32. The method of claim 22, wherein the length of the antisense strand is 28 nucleotides.

33. The method of claim 22, wherein the length of the antisense strand is 29 nucleotides.

34. The method of claim 22, wherein the length of the antisense strand is 30 nucleotides.

35. The method of any one of claims 13-34, wherein the length of the sense strand is between 12 and 30 nucleotides.

36. The method of claim 35, wherein the length of the sense strand is 14 nucleotides.

37. The method of claim 35, wherein the length of the sense strand is 15 nucleotides.

38. The method of claim 35, wherein the length of the sense strand is 16 nucleotides

39. The method of claim 35, wherein the length of the sense strand is 17 nucleotides.

40. The method of claim 35, wherein the length of the sense strand is 18 nucleotides.

41. The method of claim 35, wherein the length of the sense strand is 19 nucleotides.

42. The method of claim 35, wherein the length of the sense strand is 20 nucleotides.

43. The method of claim 35, wherein the length of the sense strand is 21 nucleotides.

44. The method of claim 35, wherein the length of the sense strand is 22 nucleotides.

45. The method of claim 35, wherein the length of the sense strand is 23 nucleotides.

46. The method of claim 35, wherein the length of the sense strand is 24 nucleotides.

47. The method of claim 35, wherein the length of the sense strand is 25 nucleotides.

48. The method of claim 35, wherein the length of the sense strand is 26 nucleotides.

49. The method of claim 35, wherein the length of the sense strand is 27 nucleotides.

50. The method of claim 35, wherein the length of the sense strand is 28 nucleotides.

51. The method of claim 35, wherein the length of the sense strand is 29 nucleotides.

52. The method of claim 35, wherein the length of the sense strand is 30 nucleotides.

53. A branched siRNA molecule comprising a sense strand and an antisense strand, wherein the antisense strand comprises a region having complementarity to a segment of contiguous nucleotides within a gene selected from the group consisting of APOE, BIN1, C1QA, C3, C9ORF72, CCL5, CD33, CLU/APOJ, CR1, CXCL10, CXCL13, IFIT1, IFIT3, IFITM3, IFNAR1, IFNAR2, IL10RA, ILIA, IL1B, IL1RAP, INPP5D, ITGAM, MEF2C, MMP12, NLRP3, NOS2, PILRA, PLCG2, PTK2B, SLC24A4, TBK1, and TNF.

54. The molecule of claim 53, wherein the antisense strand has complementarity to a portion of a gene encoding a positive regulator of a gene for which increased expression and/or activity relative to the level of expression and/or activity observed in a reference subject is associated with a disease state.

55. The molecule of claim 53, wherein the antisense strand has complementarity to a portion of a gene encoding a negative regulator of a gene for which decreased expression and/or activity relative to the level of expression and/or activity observed in a reference subject is associated with a disease state.

56. The molecule of claim 53, wherein the antisense strand has complementarity to a splice isoform of a gene for which overexpression of the splice isoform relative to the expression of the splice isoform in a reference subject is associated with a disease state.

57. The molecule of any one of claims 53-56, wherein the sense strand has complementarity to the antisense strand.

58. The molecule of any one of claims 53-57, wherein the siRNA molecule is di-branched.

59. The molecule of any one of claims 53-58, wherein the antisense strand of the branched siRNA has the following formula in the 5′-to-3′ direction:
Z-((A-P-)ₙ(B-P-)ₘ)_{q};
wherein Z is a 5′ phosphorus stabilizing moiety;
each A is, independently, a 2′-O-Me ribonucleoside;
each B is, independently, a 2′-F ribonucleoside;
each P is, independently, an internucleoside linkage selected from a phosphodiester linkage and a phosphorothioate linkage;
n is an integer from 1 to 5;
m is an integer from 1 to 5; and
q is an integer between 1 and 15.

60. The molecule of claim 59, wherein Z is represented in any one of Formula I-VIII: 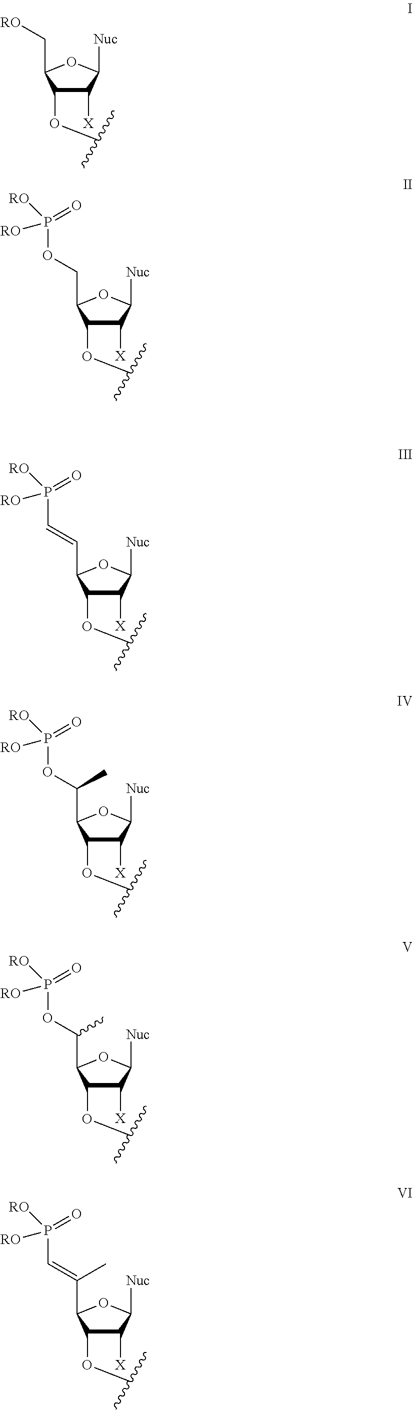 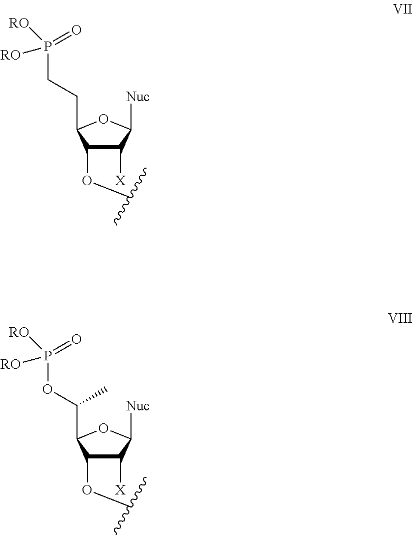
wherein Nuc represents a nucleobase selected from the group consisting of adenine, uracil, guanine, thymine, and cytosine, and R represents optionally substituted alkyl, optionally substituted alkenyl, optionally substituted alkynyl, phenyl, benzyl, hydroxy, or hydrogen.

61. The molecule of claim 59 or 60, wherein Z is (E)-vinylphosphonate as represented in Formula III.

62. The molecule of any one of claims 53-61, wherein the length of the antisense strand is between and 30 nucleotides.

63. The molecule of claim 62, wherein the length of the antisense strand is between 15 and 30 nucleotides.

64. The molecule of claim 62, wherein the length of the antisense strand is 20 nucleotides.

65. The molecule of claim 62, wherein the length of the antisense strand is 21 nucleotides.

66. The molecule of claim 62, wherein the length of the antisense strand is 22 nucleotides.

67. The molecule of claim 62, wherein the length of the antisense strand is 23 nucleotides.

68. The molecule of claim 62, wherein the length of the antisense strand is 24 nucleotides.

69. The molecule of claim 62, wherein the length of the antisense strand is 25 nucleotides.

70. The molecule of claim 62, wherein the length of the antisense strand is 26 nucleotides.

71. The molecule of claim 62, wherein the length of the antisense strand is 27 nucleotides.

72. The molecule of claim 62, wherein the length of the antisense strand is 28 nucleotides.

73. The molecule of claim 62, wherein the length of the antisense strand is 29 nucleotides.

74. The molecule of claim 62, wherein the length of the antisense strand is 30 nucleotides.

75. The molecule of any one of claims 53-74, wherein the length of the sense strand is between 12 and 30 nucleotides.

76. The molecule of claim 75, wherein the length of the sense strand is 14 nucleotides.

77. The molecule of claim 75, wherein the length of the sense strand is 15 nucleotides.

78. The molecule of claim 75, wherein the length of the sense strand is 16 nucleotides

79. The molecule of claim 75, wherein the length of the sense strand is 17 nucleotides.

80. The molecule of claim 75, wherein the length of the sense strand is 18 nucleotides.

81. The molecule of claim 75, wherein the length of the sense strand is 19 nucleotides.

82. The molecule of claim 75, wherein the length of the sense strand is 20 nucleotides.

83. The molecule of claim 75, wherein the length of the sense strand is 21 nucleotides.

84. The molecule of claim 75, wherein the length of the sense strand is 22 nucleotides.

85. The molecule of claim 75, wherein the length of the sense strand is 23 nucleotides.

86. The molecule of claim 75, wherein the length of the sense strand is 24 nucleotides.

87. The molecule of claim 75, wherein the length of the sense strand is 25 nucleotides.

88. The molecule of claim 75, wherein the length of the sense strand is 26 nucleotides.

89. The molecule of claim 75, wherein the length of the sense strand is 27 nucleotides.

90. The molecule of claim 75, wherein the length of the sense strand is 28 nucleotides.

91. The molecule of claim 75, wherein the length of the sense strand is 29 nucleotides.

92. The molecule of claim 75, wherein the length of the sense strand is 30 nucleotides.

93. A method of treating a subject diagnosed as having a disease associated with expression of a dysregulated microglial gene or dysregulated microglial gene pathway, the method comprising administering to the subject the branched siRNA molecule of any one of claims 53-92.

94. The method of claim 93, wherein the dysregulated microglial gene is selected from the group consisting of ABCA7, ABI3, ADAM10, APOC1, APOE, AXL, BIN1, C1QA, C3, C9ORF72, CASS4, CCL5, CD2AP, CD33, CD68, CLPTM1, CLU, CR1, CSF1, CST7, CTSB, CTSD, CTSL, CXCL10, CXCL13, DSG2, ECHDC3, EPHA1, FABP5, FERMT2, FTH1, GNAS, GRN, HBEGF, HLA-DRB1, HLA-DRB5, IFIT1, IFIT3, IFITM3, IFNAR1, IFNAR2, IGF1, IL10RA, ILIA, IL1B, IL1RAP, INPP5D, ITGAM, ITGAX, LILRB4, LPL, MEF2C, MMP12, MS4A4A, MS4A6A, NLRP3, NME8, NOS2, PICALM, PILRA, PLCG2, PTK2B, SCIMP, SLC24A4, SORL1, SPI1, SPP1, SPPL2A, TBK1, TNF, TREM2, TREML2, TYROBP, and ZCVVPW1.

95. The method of claim 93, wherein the dysregulated microglial gene exhibits increased expression and/or activity in microglial cells of the subject as compared to the expression and/or activity of the same gene in microglial cells of a reference subject.

96. The method of claim 93, wherein the dysregulated microglial gene exhibits reduced expression and/or activity in microglial cells of the subject as compared to the expression and/or activity of the same gene in microglial cells of a reference subject.

97. The method of claim 93, wherein the administering of the branched siRNA molecule to the subject results in silencing of a gene in the subject.

98. The method of claim 97, wherein the silencing of a gene comprises silencing any one of the genes selected from the group consisting of APOE, BIN1, C1QA, C3, C9ORF72, CCL5, CD33, CLU/APOJ, CR1, CXCL10, CXCL13, IFIT1, IFIT3, IFITM3, IFNAR1, IFNAR2, IL10RA, ILIA, IL1B, IL1RAP, INPP5D, ITGAM, MEF2C, MMP12, NLRP3, NOS2, PILRA, PLCG2, PTK2B, SLC24A4, TBK1, and TNF.

99. The method of claim 97, wherein silencing of a gene comprises silencing of a positive regulator of a gene for which increased expression and/or activity relative to the level of expression and/or activity observed in a reference subject is associated with a disease state.

100. The method of claim 97, wherein silencing of a gene comprises silencing of a gene for which decreased expression and/or activity relative to the level of expression and/or activity observed in a reference subject is associated with a disease state.

101. The method of claim 97, wherein silencing of a gene comprises silencing of a splice isoform of a gene for which overexpression of the splice isoform relative to the expression of the splice isoform in a reference subject is associated with a disease state.

102. The method of any one of claims 93-101, wherein the subject is a human.


SEQUENCE LISTING (74 sequences)

SEQ ID NO: 1 - DNA, 6588 bp, Homo sapiens
atggccttctggacacagctgatgctgctgctctggaagaatttcatgtatcgccggagacagccggtccagctcctggtcgaattgctgtggcctctcttcctcttcttcatcctggtggctgttcgccactcccacccgcccctggagcaccatgaatgccacttcccaaacaagccactgccatcggcgggcaccgtgccctggctccagggtctcatctgtaatgtgaacaacacctgctttccgcagctgacaccgggcgaggagcccgggcgcctgagcaacttcaacgactccctggtctcccggctgctagccgatgcccgcactgtgctgggaggggccagtgcccacaggacgctggctggcctagggaagctgatcgccacgctgagggctgcacgcagcacggcccagcctcaaccaaccaagcagtctccactggaaccacccatgctggatgtcgcggagctgctgacgtcactgctgcgcacggaatccctggggttggcactgggccaagcccaggagcccttgcacagcttgttggaggccgctgaggacctggcccaggagctcctggcgctgcgcagcctggtggagcttcgggcactgctgcagagaccccgagggaccagcggccccctggagttgctgtcagaggccctctgcagtgtcaggggacctagcagcacagtgggcccctccctcaactggtacgaggctagtgacctgatggagctggtggggcaggagccagaatccgccctgccagacagcagcctgagccccgcctgctcggagctgattggagccctggacagccacccgctgtcccgcctgctctggagacgcctgaagcctctgatcctcgggaagctactctttgcaccagatacaccttttacccggaagctcatggcccaggtcaaccggaccttcgaggagctcaccctgctgagggatgtccgggaggtgtgggagatgctgggaccccggatcttcaccttcatgaacgacagttccaatgtggccatgctgcagcggctcctgcagatgcaggatgaaggaagaaggcagcccagacctggaggccgggaccacatggaggccctgcgatcctttctggaccctgggagcggtggctacagctggcaggacgcacacgctgatgtggggcacctggtgggcacgctgggccgagtgacggagtgcctgtccttggacaagctggaggcggcaccctcagaggcagccctggtgtcgcgggccctgcaactgctcgcggaacatcgattctgggccggcgtcgtcttcttgggacctgaggactcttcagaccccacagagcacccaaccccagacctgggccccggccacgtgcgcatcaaaatccgcatggacattgacgtggtcacgaggaccaataagatcagggacaggttttgggaccctggcccagccgcggaccccctgaccgacctgcgctacgtgtggggcggcttcgtgtacctgcaagacctggtggagcgtgcagccgtccgcgtgctcagcggcgccaacccccgggccggcctctacctgcagcagatgccctatccgtgctatgtggacgacgtgttcctgcgtgtgctgagccggtcgctgccgctcttcctgacgctggcctggatctactccgtgacactgacagtgaaggccgtggtgcgggagaaggagacgcggctgcgggacaccatgcgcgccatggggctcagccgcgcggtgctctggctaggctggttcctcagctgcctcgggcccttcctgctcagcgccgcactgctggttctggtgctcaagctgggagacatcctcccctacagccacccgggcgtggtcttcctgttcttggcagccttcgcggtggccacggtgacccagagcttcctgctcagcgccttcttctcccgcgccaacctggctgcggcctgcggcggcctggcctacttctccctctacctgccctacgtgctgtgtgtggcttggcgggaccggctgcccgcgggtggccgcgtggccgcgagcctgctgtcgcccgtggccttcggcttcggctgcgagagcctggctctgctggaggagcagggcgagggcgcgcagtggcacaacgtgggcacccggcctacggcagacgtcttcagcctggcccaggtctctggccttctgctgctggacgcggcgctctacggcctcgccacctggtacctggaagctgtgtgcccaggccagtacgggatccctgaaccatggaattttccttttcggaggagctactggtgcggacctcggccccccaagagtccagccccttgccccaccccgctggacccaaaggtgctggtagaagaggcaccgcccggcctgagtcctggcgtctccgttcgcagcctggagaagcgctttcctggaagcccgcagccagccctgcgggggctcagcctggacttctaccagggccacatcaccgccttcctgggccacaacggggccggcaagaccaccaccctgtccatcttgagtggcctcttcccacccagtggtggctctgccttcatcctgggccacgacgtccgctccagcatggccgccatccggccccacctgggcgtctgtcctcagtacaacgtgctgtttgacatgctgaccgtggacgagcacgtctggttctatgggcggctgaagggtctgagtgccgctgtagtgggccccgagcaggaccgtctgctgcaggatgtggggctggtctccaagcagagtgtgcagactcgccacctctctggtgggatgcaacggaagctgtccgtggccattgcctttgtgggcggctcccaagttgttatcctggacgagcctacggctggcgtggatcctgcttcccgccgcggtatttgggagctgctgctcaaataccgagaaggtcgcacgctgatcctctccacccaccacctggatgaggcagagctgctgggagaccgtgtggctgtggtggcaggtggccgcttgtgctgctgtggctccccactcttcctgcgccgtcacctgggctccggctactacctgacgctggtgaaggcccgcctgcccctgaccaccaatgagaaggctgacactgacatggagggcagtgtggacaccaggcaggaaaagaagaatggcagccagggcagcagagtcggcactcctcagctgctggccctggtacagcactgggtgcccggggcacggctggtggaggagctgccacacgagctggtgctggtgctgccctacacgggtgcccatgacggcagcttcgccacactcttccgagagctagacacgcggctggcggagctgaggctcactggctacgggatctccgacaccagcctcgaggagatcttcctgaaggtggtggaggagtgtgctgcggacacagatatggaggatggcagctgcgggcagcacctatgcacaggcattgctggcctagacgtaaccctgcggctcaagatgccgccacaggagacagcgctggagaacggggaaccagctgggtcagccccagagactgaccagggctctgggccagacgccgtgggccgggtacagggctgggcactgacccgccagcagctccaggccctgcttctcaagcgctttctgcttgcccgccgcagccgccgcggcctgttcgcccagatcgtgctgcctgccctctttgtgggcctggccctcgtgttcagcctcatcgtgcctcctttcgggcactacccggctctgcggctcagtcccaccatgtacggtgctcaggtgtccttcttcagtgaggacgccccaggggaccctggacgtgcccggctgctcgaggcgctgctgcaggaggcaggactggaggagcccccagtgcagcatagctcccacaggttctcggcaccagaagttcctgctgaagtggccaaggtcttggccagtggcaactggaccccagagtctccatccccagcctgccagtgtagccagcccggtgcccggcgcctgctgcccgactgcccggctgcagctggtggtccccctccgccccaggcagtgaccggctctggggaagtggttcagaacctgacaggccggaacctgtctgacttcctggtcaagacctacccgcgcctggtgcgccagggcctgaagactaagaagtgggtgaatgaggtcaggtacggaggcttctcgctggggggccgagacccaggcctgccctcgggccaagagttgggccgctcagtggaggagttgtgggcgctgctgagtcccctgcctggcggggccctcgaccgtgtcctgaaaaacctcacagcctgggctcacagcctggacgctcaggacagtctcaagatctggttcaacaacaaaggctggcactccatggtggcctttgtcaaccgagccagcaacgcaatcctccgtgctcacctgcccccaggccgggcccgccacgcccacagcatcaccacactcaaccaccccttgaacctcaccaaggagcagctgtttgaggctgcattgatggcctcctcggtggacgtcctcgtctccatctgtgtggtctttgccatgtcctttgtcccggccagcttcactcttgtcctcattgaggagcgagtcacccgagccaagcacctgcagctcatggggggcctgtcccccaccctctactggcttggcaactttctctgggacatgtgtaactacttggtgccagcatgcatcgtggtgctcatctttctggccttccagcagagggcatatgtggcccctgccaacctgcctgctctcctgctgttgctactactgtatggctggtcgatcacaccgctcatgtacccagcctccttcttcttctccgtgcccagcacagcctatgtggtgctcacctgcataaacctctttattggcatcaatggaagcatggccacctttgtgcttgagctcttctctgatcagaagctgcaggaggtgagccggatcttgaaacaggtcttccttatcttcccccacttctgcttgggccgggggcttattgacatggtgcggaaccaggccatggctgatgcctttgagcgcttgggagacaggcagttccagtcacccctgcgctgggaggtggtcggcaagaacctcttggccatggtgatacaggggcccctcttccttctcttcacactactgctgcagcaccgaagccaactcctgccacagcccagggtgaggtctctgccactcctgggagaggaggacgaggatgtagcccgtgaacgggagcgggtggtccaaggagccacccagggggatgtgttggtgctgaggaacttgaccaaggtataccgtgggcagaggatgccagctgttgaccgcttgtgcctggggattccccctggtgagtgttttgggctgctgggtgtgaatggagcagggaagacgtccacgtttcgcatggtgacgggggacacattggccagcaggggcgaggctgtgctggcaggccacagcgtggcccgggaacccagtgctgcgcacctcagcatgggatactgccctcaatccgatgccatctttgagctgctgacgggccgcgagcacctggagctgcttgcgcgcctgcgcggtgtcccggaggcccaggttgcccagaccgctggctcgggcctggcgcgtctgggactctcatggtacgcagaccggcctgcaggcacctacagcggagggaacaaacgcaagctggcgacggccctggcgctggttggggacccagccgtggtgtttctggacgagccgaccacaggcatggaccccagcgcgcggcgcttcctttggaacagccttttggccgtggtgcgggagggccgttcagtgatgctcacctcccatagcatggaggagtgtgaagcgctctgctcgcgcctagccatcatggtgaatgggcggttccgctgcctgggcagcccgcaacatctcaagggcagattcgcggcgggtcacacactgaccctgcgggtgcccgccgcaaggtcccagccggcagcggccttcgtggcggccgagttccctgggtcggagctgcgcgaggcacatggaggccgcctgcgcttccagctgccgccgggagggcgctgcgccctggcgcgcgtctttggagagctggcggtgcacggcgcagagcacggcgtggaggacttttccgtgagccagacgatgctggaggaggtattcttgtacttctccaaggaccaggggaaggacgaggacaccgaagagcagaaggaggcaggagtgggagtggaccccgcgccaggcctgcagcaccccaaacgcgtcagccagttcctcgatgaccctagcactgccgagactgtgctctgagcctccctcccctgcggggccgcggggaggccctgggaatggcaagggcaaggtagagtgcctaggagccctggactcaggctggcagaggggctggtgccctggagaaaataaagagaaggctggagagaagccgtgcttggtgaa

SEQ ID NO: 2 - DNA, 2129 bp, Homo sapiens
tcctatccaccctccactcccctgtcccttggtgactcatccctgagcttcccaaggaagcccccaccctctgccctttcctcccgccttccatgagtggaaaatccacctccgccccctatagcaggccagcccccttcctccccagtctccgaccccatcccccagccgaccagtttcctctccaggaccagggagcaatcacagctgccccgaccttggcttcctctgctgggtgggattgggggctgggcccccaaatgggcccctggcttcccccttcctctgggcaggggacagagagacacaggctcggggagcaggactgacttcctcttgtcccggaatgagcatgcctgccctttgcaagcaggtttgggtctcacgcagaggaaaccaaaagcaataagagggagggaaggcagagcaaccaatcaagggcagggtgagactcaaaacgagcgggctccctggggagccagacagaggctgggggtgatggcggagctacagcagctgcaggagtttgagatccccactggccgggaggctctgaggggcaaccacagtgccctgctgcgggtcgctgactactgcgaggacaactatgtgcaggccacagacaagcggaaggcgctggaggagaccatggccttcactacccaggcactggccagcgtggcctaccaggtgggcaacctggccgggcacactctgcgcatgttggacctgcagggggccgccctgcggcaggtggaagcccgtgtaagcacgctgggccagatggtgaacatgcatatggagaaggtggcccgaagggagatcggcaccttagccactgtccagcggctgccccccggccagaaggtcatcgccccagagaacctaccccctctcacgccctactgcaggagacccctcaactttggctgcctggacgacattggccatgggatcaaggacctcagcacgcagctgtcaagaacaggcaccctgtctcgaaagagcatcaaggcccctgccacacccgcctccgccaccttggggagaccaccccggattcccgagccagtgcacctgccggtggtgcccgacggcagactctccgccgcctcctctgcgtcttccctggcctcggccggcagcgccgaaggtgtcggtggggcccccacgcccaaggggcaggcagcacctccagccccacctctccccagctccttggacccacctcctccaccagcagccgtcgaggtgttccagcggcctcccacgctggaggagttgtccccacccccaccggacgaagagctgcccctgccactggacctgcctcctcctccacccctggatggagatgaattggggctgcctccacccccaccaggatttgggcctgatgagcccagctgggtgcctgcctcatacttggagaaagtggtgacactgtacccatacaccagccagaaggacaatgagctctccttctctgagggcactgtcatctgtgtcactcgccgctactccgatggctggtgcgagggcgtcagctcagaggggactggattcttccctgggaactatgtggagcccagctgctgacagcccagggctctctgggcagctgatgtctgcactgagtgggtttcatgagccccaagccaaaaccagctccagtcacagctggactgggtctgcccacctcttgggctgtgagctgtgttctgtccttcctcccatcggagggagaaggggtcctggggagagagaatttatccagaggcctgctgcagatggggaagagctggaaaccaagaagtttgtcaacagaggacccctactccatgcaggacagggtctcctgctgcaagtcccaactttgaataaaacagatgatgtcctgtgactgccccacagagataaggggccaggagggattgaaaggcatcccagttctaaggctgctgctaattacagcccccaacctccaacccaccagctgacctagaagcagcatcttcccatttcctcagtacccacaaagtgcagcccacattggaccccagacacccctctgcagccattgactgcaacttgttcttttgcccattaaaaaaaaaaaaaaaaaaaaa

SEQ ID NO: 3 - DNA, 5050 bp, Homo sapiens
gcggcggcaggcctagcagcacgggaaccgtcccccgcgcgcatgcgcgcgcccctgaagcgcctgggggacgggtaggggcgggaggtaggggcgcggctccgcgtgccagttgggtgcccgcgcgtcacgtggtgaggaaggaggcggaggtctgagtttcgaaggagggggggagagaagagggaacgagcaagggaaggaaagcggggaaaggaggaaggaaacgaacgagggggagggaggtccctgttttggaggagctaggagcgttgccggcccctgaagtggagcgagagggaggtgcttcgccgtttctcctgccaggggaggtcccggcttcccgtggaggctccggaccaagccccttcagcttctccctccggatcgatgtgctgctgttaacccgtgaggaggcggcggcggcggcagcggcagcggaagatggtgttgctgagagtgttaattctgctcctctcctgggcggcggggatgggaggtcagtatgggaatcctttaaataaatatatcagacattatgaaggattatcttacaatgtggattcattacaccaaaaacaccagcgtgccaaaagagcagtctcacatgaagaccaatttttacgtctagatttccatgcccatggaagacatttcaacctacgaatgaagagggacacttcccttttcagtgatgaatttaaagtagaaacatcaaataaagtacttgattatgatacctctcatatttacactggacatatttatggtgaagaaggaagttttagccatgggtctgttattgatggaagatttgaaggattcatccagactcgtggtggcacattttatgttgagccagcagagagatatattaaagaccgaactctgccatttcactctgtcatttatcatgaagatgatattaactatccccataaatacggtcctcaggggggctgtgcagatcattcagtatttgaaagaatgaggaaataccagatgactggtgtagaggaagtaacacagatacctcaagaagaacatgctgctaatggtccagaacttctgaggaaaaaacgtacaacttcagctgaaaaaaatacttgtcagctttatattcagactgatcatttgttctttaaatattacggaacacgagaagctgtgattgcccagatatccagtcatgttaaagcgattgatacaatttaccagaccacagacttctccggaatccgtaacatcagtttcatggtgaaacgcataagaatcaatacaactgctgatgagaaggaccctacaaatcctttccgtttcccaaatattggtgtggagaagtttctggaattgaattctgagcagaatcatgatgactactgtttggcctatgtcttcacagaccgagattttgatgatggcgtacttggtctggcttgggttggagcaccttcaggaagctctggaggaatatgtgaaaaaagtaaactctattcagatggtaagaagaagtccttaaacactggaattattactgttcagaactatgggtctcatgtacctcccaaagtctctcacattacttttgctcacgaagttggacataactttggatccccacatgattctggaacagagtgcacaccaggagaatctaagaatttgggtcaaaaagaaaatggcaattacatcatgtatgcaagagcaacatctggggacaaacttaacaacaataaattctcactctgtagtattagaaatataagccaagttcttgagaagaagagaaacaactgttttgttgaatctggccaacctatttgtggaaatggaatggtagaacaaggtgaagaatgtgattgtggctatagtgaccagtgtaaagatgaatgctgcttcgatgcaaatcaaccagagggaagaaaatgcaaactgaaacctgggaaacagtgcagtccaagtcaaggtccttgttgtacagcacagtgtgcattcaagtcaaagtctgagaagtgtcgggatgattcagactgtgcaagggaaggaatatgtaatggcttcacagctctctgcccagcatctgaccctaaaccaaacttcacagactgtaataggcatacacaagtgtgcattaatgggcaatgtgcaggttctatctgtgagaaatatggcttagaggagtgtacgtgtgccagttctgatggcaaagatgataaagaattatgccatgtatgctgtatgaagaaaatggacccatcaacttgtgccagtacagggtctgtgcagtggagtaggcacttcagtggtcgaaccatcaccctgcaacctggatccccttgcaacgattttagaggttactgtgatgttttcatgcggtgcagattagtagatgctgatggtcctctagctaggcttaaaaaagcaatttttagtccagagctctatgaaaacattgctgaatggattgtggctcattggtgggcagtattacttatgggaattgctctgatcatgctaatggctggatttattaagatatgcagtgttcatactccaagtagtaatccaaagttgcctcctcctaaaccacttccaggcactttaaagaggaggagacctccacagcccattcagcaaccccagcgtcagcggccccgagagagttatcaaatgggacacatgagacgctaactgcagcttttgccttggttcttcctagtgcctacaatgggaaaacttcactccaaagagaaacctattaagtcatcatctccaaactaaaccctcacaagtaacagttgaagaaaaaatggcaagagatcatatcctcagaccaggtggaattacttaaattttaaagcctgaaaattccaatttgggggtgggaggtggaaaaggaacccaattttcttatgaacagatatttttaacttaatggcacaaagtcttagaatattattatgtgccccgtgttccctgttcttcgttgctgcattttcttcacttgcaggcaaacttggctctcaataaacttttaccacaaattgaaataaatatatttttttcaactgccaatcaaggctaggaggctcgaccacctcaacattggagacatcacttgccaatgtacataccttgttatatgcagacatgtatttcttacgtacactgtacttctgtgtgcaattgtaaacagaaattgcaatatggatgtttctttgtattataaaatttttccgctcttaattaaaaattactgtttaattgacatactcaggataacagagaatggtggtattcagtggtccaggattctgtaatgctttacacaggcagttttgaaatgaaaatcaatttacctttctgttacgatggagttggttttgatactcattttttctttatcacatggctgctacgggcacaagtgactatactgaagaacacagttaagtgttgtgcaaactggacatagcagcacatactacttcagagttcatgatgtagatgtctggtttctgcttacgtcttttaaactttctaattcaattccatttttcaattaataggtgaaattttattcatgctttgatagaaattatgtcaatgaaatgattctttttatttgtagcctacttatttgtgtttttcatatatctgaaatatgctaattatgttttctgtctgatatggaaaagaaaagctgtgtctttatcaaaatatttaaacggttttttcagcatatcatcactgatcattggtaaccactaaagatgagtaatttgcttaagtagtagttaaaattgtagataggccttctgacattttttttcctaaaatttttaacagcattgaaggtgaaacagcacaatgtcccattccaaatttatttttgaaacagatgtaaataattggcattttaaagagaaagcaaaaacatttaatgtattaacaggcttattgctatgcaggaaatagaaggggcattacaaaaattgaagcttgtgacatatttattgcttctgttttccaactacatcacttcaactagaagtaaagctatgattttcctgacttcacataggaggcaaatttagagaaagttgtaaagatttctatgttttgggtttttttttttcctttttttttttaagagtataaggtttacacaatcattctcataatgtgacgcaagccagcaaggccaaaaatgctagagaaaataacgggatctcttccttgtaaacttgtacagtatgtggtgactttttcaaaatacagctttttgtacatgatttagagacaaattttgtacatgaaaccccagatagactataaataattctaaacaaacaagtaggtagatatgtatgtaattgcttttaaatcatttaaatgcctttgtttttggactgtgcaaaggttggaagtgggtttgcatttctaaaatggtgacttttattctgcaagagttcttagtaacttcttgagtgtggtagactttggaacatgtaaattttttgcttgtaatgttatcctgtggtaggattttggcaggtacacacactgccctattttattttgagtctaagttaaatgttttctgaaaagagatacatgcactgaactctttccactgcgaatcaagatgtggtaatataaaaggatcaagacaaatgagatctaatactactgtcagttttaatgtccactgtgttttatacagtatctttttttgttcactttggaaatttttactaaaaattgcaaaaaataaagtattgtgcaaagatgtaaggttttttgaaacttgaaatgcattaataaatagacgattaaatcaacttgaaggttctatactctttgaactctgagaactatcacaagaagcttcccacaaggcagtgttttcttacagttgtctcttcctacaaaagtatagattatctttattcttaatactttggaatccatgtagaaaatttccagttagatactctgcgtacacacaataaacctttttaaaacacccaaaaaaaaaaaaaaaaaa

SEQ ID NO: 4 - DNA, 588 bp, Homo sapiens
aacgctcacgggacaggggcagaggagaaaaacgtgggtggacagagggaggcaggcggtcaggggaaggctcaggaggagggagatcaacatcaacctgccccgccccctccccagcctgataaaggtcctgcgggcaggacaggacctcccaaccaagccctccagcaaggattcagagtgcccctccggcctcgccatgaggctcttcctgtcgctcccggtcctggtggtggttctgtcgatcgtcttggaaggcccagccccagcccaggggaccccagacgtctccagtgccttggataagctgaaggagtttggaaacacactggaggacaaggctcgggaactcatcagccgcatcaaacagagtgaactttctgccaagatgcgggagtggttttcagagacatttcagaaagtgaaggagaaactcaagattgactcatgaggacctgaagggtgacatcccaggaggggcctctgaaatttcccacaccccagcgcctgtgctgaggactccctccatgtggccccaggtgccaccaataaaaatcctacagaaaattcaaaaaaaaaaaaaaaaaa

SEQ ID NO: 5 - DNA, 1157 bp, Homo sapiens
ccccagcggaggtgaaggacgtccttccccaggagccgactggccaatcacaggcaggaagatgaaggttctgtgggctgcgttgctggtcacattcctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaagtcacccaagaactgagggcgctgatggacgagaccatgaaggagttgaaggcctacaaatcggaactggaggaacaactgaccccggtagcggaggagacgcgggcacggctgtccaaggagctgcagacggcgcaggcccggctgggcgcggacatggaggacgtgtgcggccgcctggtgcagtaccgcggcgaggtgcaggccatgctcggccagagcaccgaggagctgcgggtgcgcctcgcctcccacctgcgcaagctgcgtaagcggctcctccgcgatcccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagtcggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactgaacgccgaagcctgcagccatgcgaccccacgccaccccgtgcctcctgcctccgcgcagcctgcagcgggagaccctgtccccgccccagccgtcctcctggggtggaccctagtttaataaagattcaccaagtttcacgc

SEQ ID NO: 6 - DNA, 3227 bp, Homo sapiens
gctgggcaaagccggtggcaagggcctcccctgccgctgtgccaggcaggcagtgccaaatccggggagcctggagctggggggagggccggggacagcccggccctgccccctcccccgctgggagcccagcaacttctgaggaaagtttggcacccatggcgtggcggtgccccaggatgggcagggtcccgctggcctggtgcttggcgctgtgcggctgggcgtgcatggcccccaggggcacgcaggctgaagaaagtcccttcgtgggcaacccagggaatatcacaggtgcccggggactcacgggcacccttcggtgtcagctccaggttcagggagagccccccgaggtacattggcttcgggatggacagatcctggagctcgcggacagcacccagacccaggtgcccctgggtgaggatgaacaggatgactggatagtggtcagccagctcagaatcacctccctgcagctttccgacacgggacagtaccagtgtttggtgtttctgggacatcagaccttcgtgtcccagcctggctatgttgggctggagggcttgccttacttcctggaggagcccgaagacaggactgtggccgccaacacccccttcaacctgagctgccaagctcagggacccccagagcccgtggacctactctggctccaggatgctgtccccctggccacggctccaggtcacggcccccagcgcagcctgcatgttccagggctgaacaagacatcctctttctcctgcgaagcccataacgccaagggggtcaccacatcccgcacagccaccatcacagtgctcccccagcagccccgtaacctccacctggtctcccgccaacccacggagctggaggtggcttggactccaggcctgagcggcatctaccccctgacccactgcaccctgcaggctgtgctgtcagacgatgggatgggcatccaggcgggagaaccagaccccccagaggagcccctcacctcgcaagcatccgtgcccccccatcagcttcggctaggcagcctccatcctcacaccccttatcacatccgcgtggcatgcaccagcagccagggcccctcatcctggacccactggcttcctgtggagacgccggagggagtgcccctgggcccccctaagaacattagtgctacgcggaatgggagccaggccttcgtgcattggcaagagccccgggcgcccctgcagggtaccctgttagggtaccggctggcgtatcaaggccaggacaccccagaggtgctaatggacatagggctaaggcaagaggtgaccctggagctgcagggggacgggtctgtgtccaatctgacagtgtgtgtggcagcctacactgctgctggggatggaccctggagcctcccagtacccctggaggcctggcgcccagtgaaggaaccttcaactcctgccttctcgtggccctggtggtatgtactgctaggagcagtcgtggccgctgcctgtgtcctcatcttggctctcttccttgtccaccggcgaaagaaggagacccgttatggagaagtgtttgaaccaacagtggaaagaggtgaactggtagtcaggtaccgcgtgcgcaagtcctacagtcgtcggaccactgaagctaccttgaacagcctgggcatcagtgaagagctgaaggagaagctgcgggatgtgatggtggaccggcacaaggtggccctggggaagactctgggagagggagagtttggagctgtgatggaaggccagctcaaccaggacgactccatcctcaaggtggctgtgaagacgatgaagattgccatctgcacgaggtcagagctggaggatttcctgagtgaagcggtctgcatgaaggaatttgaccatcccaacgtcatgaggctcatcggtgtctgtttccagggttctgaacgagagagcttcccagcacctgtggtcatcttacctttcatgaaacatggagacctacacagcttcctcctctattcccggctcggggaccagccagtgtacctgcccactcagatgctagtgaagttcatggcagacatcgccagtggcatggagtatctgagtaccaagagattcatacaccgggacctggcggccaggaactgcatgctgaatgagaacatgtccgtgtgtgtggcggacttcgggctctccaagaagatctacaatggggactactaccgccagggacgtatcgccaagatgccagtcaagtggattgccattgagagtctagctgaccgtgtctacaccagcaagagcgatgtgtggtccttcggggtgacaatgtgggagattgccacaagaggccaaaccccatatccgggcgtggagaacagcgagatttatgactatctgcgccagggaaatcgcctgaagcagcctgcggactgtctggatggactgtatgccttgatgtcgcggtgctgggagctaaatccccaggaccggccaagttttacagagctgcgggaagatttggagaacacactgaaggccttgcctcctgcccaggagcctgacgaaatcctctatgtcaacatggatgagggtggaggttatcctgaaccccctggagctgcaggaggagctgaccccccaacccagccagaccctaaggattcctgtagctgcctcactgcggctgaggtccatcctgctggacgctatgtcctctgcccttccacaacccctagccccgctcagcctgctgataggggctccccagcagccccagggcaggaggatggtgcctgagacaaccctccacctggtactccctctcaggatccaagctaagcactgccactggggaaaactccaccttcccacttttccaccccacgccttatccccacttgcagccctgtcttcctacctatcccacctccatcccagacaggtccctccccttctctgtgcagtagcatcaccttgaaagcagtagcatcaccatctgtaaaaggaaggggttggattgcaatatctgaagccctcccaggtgttaacattccaagactctagagtccaaggtttaaagagtctagattcaaaggttctaggtttcaaagatgctgtgagtctttggttctaaggacctgaaattccaaagtctctaattctattaaagtgctaaggttctaaggcaaaaaaaaaaaaaaaaaaaaa

SEQ ID NO: 7 - DNA, 1782 bp, Homo sapiens
atggcagagatgggcagtaaaggggtgacggcgggaaagatcgccagcaacgtgcagaagaagctcacccgcgcgcaggagaaggttctccagaagctggggaaggcagatgagaccaaggatgagcagtttgagcagtgcgtccagaatttcaacaagcagctgacggagggcacccggctgcagaaggatctccggacctacctggcctccgtcaaagccatgcacgaggcttccaagaagctgaatgagtgtctgcaggaggtgtatgagcccgattggcccggcagggatgaggcaaacaagatcgcagagaacaacgacctgctgtggatggattaccaccagaagctggtggaccaggcgctgctgaccatggacacgtacctgggccagttccccgacatcaagtcacgcattgccaagcgggggcgcaagctggtggactacgacagtgcccggcaccactacgagtcccttcaaaccgccaaaaagaaggatgaagccaaaattgccaagcctgtctcgctgcttgagaaagccgccccccagtggtgccaaggcaaactgcaggctcatctcgtagctcaaactaacctgctccgaaatcaggccgaggaggagctcatcaaagcccagaaggtgtttgaggagatgaatgtggatctgcaggaggagctgccgtccctgtggaacagccgcgtaggtttctacgtcaacacgttccagagcatcgcgggcctggaggaaaacttccacaaggagatgagcaagctcaaccagaacctcaatgatgtgctggtcggcctggagaagcaacacgggagcaacaccttcacggtcaaggcccagcccagtgacaacgcgcctgcaaaagggaacaagagcccttcgcctccagatggctcccctgccgccacccccgagatcagagtcaaccacgagccagagccggccggcggggccacgcccggggccaccctccccaagtccccatctcagctccggaaaggcccaccagtccctccgcctcccaaacacaccccgtccaaggaagtcaagcaggagcagatcctcagcctgtttgaggacacgtttgtccctgagatcagcgtgaccaccccctcccagtttgaggccccggggcctttctcggagcaggccagtctgctggacctggactttgaccccctcccgcccgtgacgagccctgtgaaggcacccacgccctctggtcagtcaattccatgggacctctgggagcccacagagagtccagccggcagcctgccttccggggagcccagcgctgccgagggcacctttgctgtgtcctggcccagccagacggccgagccggggcctgcccaaccagcagaggcctcggaggtggcgggtgggacccaacctgcggctggagcccaggagccaggggagacggcggcaagtgaagcagcctccagctctcttcctgctgtcgtggtggagaccttcccagcaactgtgaatggcaccgtggagggcggcagtggggccgggcgcttggacctgcccccaggtttcatgttcaaggtacaggcccagcacgactacacggccactgacacagacgagctgcagctcaaggctggtgatgtggtgctggtgatccccttccagaaccctgaagagcaggatgaaggctggctcatgggcgtgaaggagagcgactggaaccagcacaaggagctggagaagtgccgtggcgtcttccccgagaacttcactgagagggtcccatga

SEQ ID NO: 8 - DNA, 1228 bp, Homo sapiens
agtcttgctgaagtctgcttgaaatgtccctggtgagcttctggccactggggaagttcagggggcaggtctgaagaaggggaagtaggaagggatgtgaaacttggccacagcctggagccactcctgctgggcagcccacagggtccctgggcggagggcaggagcatccagttggagttgacaacaggaggcagaggcatcatggagggtccccggggatggctggtgctctgtgtgctggccatatcgctggcctctatggtgaccgaggacttgtgccgagcaccagacgggaagaaaggggaggcaggaagacctggcagacgggggcggccaggcctcaagggggagcaaggggagccgggggcccctggcatccggacaggcatccaaggccttaaaggagaccagggggaacctgggccctctggaaaccccggcaaggtgggctacccagggcccagcggccccctcggagcccgtggcatcccgggaattaaaggcaccaagggcagcccaggaaacatcaaggaccagccgaggccagccttctccgccattcggcggaaccccccaatggggggcaacgtggtcatcttcgacacggtcatcaccaaccaggaagaaccgtaccagaaccactccggccgattcgtctgcactgtacccggctactactacttcaccttccaggtgctgtcccagtgggaaatctgcctgtccatcgtctcctcctcaaggggccaggtccgacgctccctgggcttctgtgacaccaccaacaaggggctcttccaggtggtgtcagggggcatggtgcttcagctgcagcagggtgaccaggtctgggttgaaaaagaccccaaaaagggtcacatttaccagggctctgaggccgacagcgtcttcagcggcttcctcatcttcccatctgcctgagccagggaaggaccccctcccccacccacctctctggcttccatgctccgcctgtaaaatgggggcgctattgcttcagctgctgaagggagggggctggctctgagagccccaggactggctgccccgtgacacatgctctaagaagctcgtttcttagacctcttcctggaataaacatctgtgtctgtgtctgctgaacatgagcttcagttgctactcggagcattgagagggaggcctaagaataataacaatccagtgcttaagagtcaaaaaaaaaaaa

SEQ ID NO: 9 - DNA, 5148 bp, Homo sapiens
agataaaaagccagctccagcaggcgctgctcactcctccccatcctctccctctgtccctctgtccctctgaccctgcactgtcccagcaccatgggacccacctcaggtcccagcctgctgctcctgctactaacccacctccccctggctctggggagtcccatgtactctatcatcacccccaacatcttgcggctggagagcgaggagaccatggtgctggaggcccacgacgcgcaaggggatgttccagtcactgttactgtccacgacttcccaggcaaaaaactagtgctgtccagtgagaagactgtgctgacccctgccaccaaccacatgggcaacgtcaccttcacgatcccagccaacagggagttcaagtcagaaaaggggcgcaacaagttcgtgaccgtgcaggccaccttcgggacccaagtggtggagaaggtggtgctggtcagcctgcagagcgggtacctcttcatccagacagacaagaccatctacacccctggctccacagttctctatcggatcttcaccgtcaaccacaagctgctacccgtgggccggacggtcatggtcaacattgagaacccggaaggcatcccggtcaagcaggactccttgtcttctcagaaccagcttggcgtcttgcccttgtcttgggacattccggaactcgtcaacatgggccagtggaagatccgagcctactatgaaaactcaccacagcaggtcttctccactgagtttgaggtgaaggagtacgtgctgcccagtttcgaggtcatagtggagcctacagagaaattctactacatctataacgagaagggcctggaggtcaccatcaccgccaggttcctctacgggaagaaagtggagggaactgcctttgtcatcttcgggatccaggatggcgaacagaggatttccctgcctgaatccctcaagcgcattccgattgaggatggctcgggggaggttgtgctgagccggaaggtactgctggacggggtgcagaacccccgagcagaagacctggtggggaagtctttgtacgtgtctgccaccgtcatcttgcactcaggcagtgacatggtgcaggcagagcgcagcgggatccccatcgtgacctctccctaccagatccacttcaccaagacacccaagtacttcaaaccaggaatgccctttgacctcatggtgttcgtgacgaaccctgatggctctccagcctaccgagtccccgtggcagtccagggcgaggacactgtgcagtctctaacccagggagatggcgtggccaaactcagcatcaacacacaccccagccagaagcccttgagcatcacggtgcgcacgaagaagcaggagctctcggaggcagagcaggctaccaggaccatgcaggctctgccctacagcaccgtgggcaactccaacaattacctgcatctctcagtgctacgtacagagctcagacccggggagaccctcaacgtcaacttcctcctgcgaatggaccgcgcccacgaggccaagatccgctactacacctacctgatcatgaacaagggcaggctgttgaaggcgggacgccaggtgcgagagcccggccaggacctggtggtgctgcccctgtccatcaccaccgacttcatcccttccttccgcctggtggcgtactacacgctgatcggtgccagcggccagagggaggtggtggccgactccgtgtgggtggacgtcaaggactcctgcgtgggctcgctggtggtaaaaagcggccagtcagaagaccggcagcctgtacctgggcagcagatgaccctgaagatagagggtgaccacggggcccgggtggtactggtggccgtggacaagggcgtgttcgtgctgaataagaagaacaaactgacgcagagtaagatctgggacgtggtggagaaggcagacatcggctgcaccccgggcagtgggaaggattacgccggtgtcttctccgacgcagggctgaccttcacgagcagcagtggccagcagaccgcccagagggcagaacttcagtgcccgcagccagccgcccgccgacgccgttccgtgcagctcacggagaagcgaatggacaaagtcggcaagtaccccaaggagctgcgcaagtgctgcgaggacggcatgcgggagaaccccatgaggttctcgtgccagcgccggacccgtttcatctccctgggcgaggcgtgcaagaaggtcttcctggactgctgcaactacatcacagagctgcggcggcagcacgcgcgggccagccacctgggcctggccaggagtaacctggatgaggacatcattgcagaagagaacatcgtttcccgaagtgagttcccagagagctggctgtggaacgttgaggacttgaaagagccaccgaaaaatggaatctctacgaagctcatgaatatatttttgaaagactccatcaccacgtgggagattctggctgtgagcatgtcggacaagaaagggatctgtgtggcagaccccttcgaggtcacagtaatgcaggacttcttcatcgacctgcggctaccctactctgttgttcgaaacgagcaggtggaaatccgagccgttctctacaattaccggcagaaccaagagctcaaggtgagggtggaactactccacaatccagccttctgcagcctggccaccaccaagaggcgtcaccagcagaccgtaaccatcccccccaagtcctcgttgtccgttccatatgtcatcgtgccgctaaagaccggcctgcaggaagtggaagtcaaggctgctgtctaccatcatttcatcagtgacggtgtcaggaagtccctgaaggtcgtgccggaaggaatcagaatgaacaaaactgtggctgttcgcaccctggatccagaacgcctgggccgtgaaggagtgcagaaagaggacatcccacctgcagacctcagtgaccaagtcccggacaccgagtctgagaccagaattctcctgcaagggaccccagtggcccagatgacagaggatgccgtcgacgcggaacggctgaagcacctcattgtgaccccctcgggctgcggggaacagaacatgatcggcatgacgcccacggtcatcgctgtgcattacctggatgaaacggagcagtgggagaagttcggcctagagaagcggcagggggccttggagctcatcaagaaggggtacacccagcagctggccttcagacaacccagctctgcctttgcggccttcgtgaaacgggcacccagcacctggctgaccgcctacgtggtcaaggtcttctctctggctgtcaacctcatcgccatcgactcccaagtcctctgcggggctgttaaatggctgatcctggagaagcagaagcccgacggggtcttccaggaggatgcgcccgtgatacaccaagaaatgattggtggattacggaacaacaacgagaaagacatggccctcacggcctttgttctcatctcgctgcaggaggctaaagatatttgcgaggagcaggtcaacagcctgccaggcagcatcactaaagcaggagacttccttgaagccaactacatgaacctacagagatcctacactgtggccattgctggctatgctctggcccagatgggcaggctgaaggggcctcttcttaacaaatttctgaccacagccaaagataagaaccgctgggaggaccctggtaagcagctctacaacgtggaggccacatcctatgccctcttggccctactgcagctaaaagactttgactttgtgcctcccgtcgtgcgttggctcaatgaacagagatactacggtggtggctatggctctacccaggccaccttcatggtgttccaagccttggctcaataccaaaaggacgcccctgaccaccaggaactgaaccttgatgtgtccctccaactgcccagccgcagctccaagatcacccaccgtatccactgggaatctgccagcctcctgcgatcagaagagaccaaggaaaatgagggtttcacagtcacagctgaaggaaaaggccaaggcaccttgtcggtggtgacaatgtaccatgctaaggccaaagatcaactcacctgtaataaattcgacctcaaggtcaccataaaaccagcaccggaaacagaaaagaggcctcaggatgccaagaacactatgatccttgagatctgtaccaggtaccggggagaccaggatgccactatgtctatattggacatatccatgatgactggctttgctccagacacagatgacctgaagcagctggccaatggtgttgacagatacatctccaagtatgagctggacaaagccttctccgataggaacaccctcatcatctacctggacaaggtctcacactctgaggatgactgtctagctttcaaagttcaccaatactttaatgtagagcttatccagcctggagcagtcaaggtctacgcctattacaacctggaggaaagctgtacccggttctaccatccggaaaaggaggatggaaagctgaacaagctctgccgtgatgaactgtgccgctgtgctgaggagaattgcttcatacaaaagtcggatgacaaggtcaccctggaagaacggctggacaaggcctgtgagccaggagtggactatgtgtacaagacccgactggtcaaggttcagctgtccaatgactttgacgagtacatcatggccattgagcagaccatcaagtcaggctcggatgaggtgcaggttggacagcagcgcacgttcatcagccccatcaagtgcagagaagccctgaagctggaggagaagaaacactacctcatgtggggtctctcctccgatttctggggagagaagcccaacctcagctacatcatcgggaaggacacttgggtggagcactggcccgaggaggacgaatgccaagacgaagagaaccagaaacaatgccaggacctcggcgccttcaccgagagcatggttgtctttgggtgccccaactgaccacacccccattcccccactccagataaagcttcagttatatctcaaaaaaaaaaaaaaaaa

SEQ ID NO: 10 - DNA, 3316 bp, Homo sapiens
aggaaagagaggtgcgtcaaacagcgacaagttccgcccacgtaaaagatgacgcttggtgtgtcagccgtccctgctgcccggttgcttctcttttgggggcggggtctagcaagagcaggtgtgggtttaggagatatctccggagcatttggataatgtgacagttggaatgcagtgatgtcgactctttgcccaccgccatctccagctgttgccaagacagagattgctttaagtggcaaatcacctttattagcagctacttttgcttactgggacaatattcttggtcctagagtaaggcacatttgggctccaaagacagaacaggtacttctcagtgatggagaaataacttttcttgccaaccacactctaaatggagaaatccttcgaaatgcagagagtggtgctatagatgtaaagttttttgtcttgtctgaaaagggagtgattattgtttcattaatctttgatggaaactggaatggggatcgcagcacatatggactatcaattatacttccacagacagaacttagtttctacctcccacttcatagagtgtgtgttgatagattaacacatataatccggaaaggaagaatatggatgcataaggaaagacaagaaaatgtccagaagattatcttagaaggcacagagagaatggaagatcagggtcagagtattattccaatgcttactggagaagtgattcctgtaatggaactgctttcatctatgaaatcacacagtgttcctgaagaaatagatatagctgatacagtactcaatgatgatgatattggtgacagctgtcatgaaggctttcttctcaatgccatcagctcacacttgcaaacctgtggctgttccgttgtagtaggtagcagtgcagagaaagtaaataagatagtcagaacattatgcctttttctgactccagcagagagaaaatgctccaggttatgtgaagcagaatcatcatttaaatatgagtcagggctctttgtacaaggcctgctaaaggattcaactggaagctttgtgctgcctttccggcaagtcatgtatgctccatatcccaccacacacatagatgtggatgtcaatactgtgaagcagatgccaccctgtcatgaacatatttataatcagcgtagatacatgagatccgagctgacagccttctggagagccacttcagaagaagacatggctcaggatacgatcatctacactgacgaaagctttactcctgatttgaatatttttcaagatgtcttacacagagacactctagtgaaagccttcctggatcaggtctttcagctgaaacctggcttatctctcagaagtactttccttgcacagtttctacttgtccttcacagaaaagccttgacactaataaaatatatagaagacgatacgcagaagggaaaaaagccctttaaatctcttcggaacctgaagatagaccttgatttaacagcagagggcgatcttaacataataatggctctggctgagaaaattaaaccaggcctacactcttttatctttggaagacctttctacactagtgtgcaagaacgagatgttctaatgactttttaaatgtgtaacttaataagcctattccatcacaatcatgatcgctggtaaagtagctcagtggtgtggggaaacgttcccctggatcatactccagaattctgctctcagcaattgcagttaagtaagttacactacagttctcacaagagcctgtgaggggatgtcaggtgcatcattacattgggtgtctcttttcctagatttatgcttttgggatacagacctatgtttacaatataataaatattattgctatcttttaaagatataataataggatgtaaacttgaccacaactactgtttttttgaaatacatgattcatggtttacatgtgtcaaggtgaaatctgagttggcttttacagatagttgactttctatcttttggcattctttggtgtgtagaattactgtaatacttctgcaatcaactgaaaactagagcctttaaatgatttcaattccacagaaagaaagtgagcttgaacataggatgagctttagaaagaaaattgatcaagcagatgtttaattggaattgattattagatcctactttgtggatttagtccctgggattcagtctgtagaaatgtctaatagttctctatagtccttgttcctggtgaaccacagttagggtgttttgtttattttattgttcttgctattgttgatattctatgtagttgagctctgtaaaaggaaattgtattttatgttttagtaattgttgccaactttttaaattaattttcattatttttgagccaaattgaaatgtgcacctcctgtgccttttttctccttagaaaatctaattacttggaacaagttcagatttcactggtcagtcattttcatcttgttttcttcttgctaagtcttaccatgtacctgctttggcaatcattgcaactctgagattataaaatgccttagagaatatactaactaataagatctttttttcagaaacagaaaatagttccttgagtacttccttcttgcatttctgcctatgtttttgaagttgttgctgtttgcctgcaataggctataaggaatagcaggagaaattttactgaagtgctgttttcctaggtgctactttggcagagctaagttatcttttgttttcttaatgcgtttggaccattttgctggctataaaataactgattaatataattctaacacaatgttgacattgtagttacacaaacacaaataaatattttatttaaaattctggaagtaatataaaagggaaaatatatttataagaaagggataaaggtaatagagcccttctgccccccacccaccaaatttacacaacaaaatgacatgttcgaatgtgaaaggtcataatagctttcccatcatgaatcagaaagatgtggacagcttgatgttttagacaaccactgaactagatgactgttgtactgtagctcagtcatttaaaaaatatataaatactaccttgtagtgtcccatactgtgttttttacatggtagattcttatttaagtgctaactggttattttctttggctggtttattgtactgttatacagaatgtaagttgtacagtgaaataagttattaaagcatgtgtaaacattgttatatatcttttctcctaaatggagaattttgaataaaatatatttgaaatttt

SEQ ID NO: 11 - DNA, 2616 bp, Homo sapiens
gaagagtggtgtttttttcttcttcttcttcttttgtggtttcacatagcaaatgagtgacagtctctacttacagacaaagtgagacgtcaggcattgagacatagctccatagaattcagtttctgagaaccagccagaagcatgcagtgacattgcacaatctgcctctgaagctggagatactagctgcagagctcaggggagctgctccacatcaccgacatgaagggaacaggcatcatggactgtgcgcccaaggcactcctggccagggcactttatgacaactgccctgactgctctgacgagctggctttcagcagaggggacatcctgaccattctggagcaacacgtgccagaaagcgagggttggtggaagtgtttgctccatgggaggcaaggcctggcccctgccaaccgcctccaaatcctcacggaggtcgctgcagacaggccgtgccccccattcctgagaggcctggaagaagctcctgccagctcagaggagacctatcaggtgcccactctaccccgccctcccactccaggccccgtttatgagcagatgaggagttgggcggaggggccccagccccctactgcccaagtctatgaattccccgaccctcccaccagtgccagaatcatctgtgaaaagactctcagctttccaaaacaggccatcctcacgcttcccagacctgtccgggcctcactgccgactctgccttcccaggtgtatgacgtgcctacccagcaccggggccccgtggtcctgaaggagccagagaagcagcagttatatgacataccagccagccccaagaaggcaggactccatcccccagacagccaagcaagtgggcagggtgttcccctgatatcagtgactaccttaagaagaggcggttacagcacattaccaaatcctcagaaatcggaatggatttatgacactccagtgtctccaggaaaggccagcgtcagaaacacgcctctcaccagctttgcggaagaatcaaggccccacgctctccccagttccagctccactttctacaatcctccaagtggcagatccaggtccctcactccacaactgaataacaatgtgcccatgcagaaaaaactcagccttccagaaattccttcttatggctttcttgtacccagaggcacatttcctttggatgaagatgtcagcaacaaggttccttcaagcttctctgattccccgagtggacagcagaacaccaagcccaatatagacatccctaaagcaacgtcgagtgtttctcaggctgggaaggagctggagaaagccaaggaggtgtcagagaattccgcgggccataattcctcatggttctccagacggacaacttccccatctcctgaaccggacagattatcaggttccagttctgacagcagagctagcatcgtttcctcgtgctccaccacatccaccgacgactcctccagctcttcctcggaggagtcagcaaaggagctctccttggacctggatgtggccaaggagacagtgatggctctgcagcacaaggtggtcagctctgtcgctggcctgatgctctttgtcagcaggaagtggagattccgagactatctggaggccaacattgatgcaatccacaggtccactgatcacatagaagcctctgtaagagaatttctggattttgcccgaggagtccatgggactgcctgtaacctcactgacagtaaccttcagaacagaattcgggaccagatgcagaccatctccaactcctaccgcatcctgcttgaaacaaaggaaagcttggataatcgcaattggcctctggaagttcttgtgactgacagtgtccagaacagcccagatgaccttgagaggtttgtcatggtggcacggatgcttccagaagacatcaagaggtttgcctccattgtcattgccaatggaaggctcctttttaagcggaactgtgaaaaggaagagactgtgcagttgaccccaaatgcagaatttaagtgtgaaaaatacatccagcctccccaaagagaaactgaatcacaccaaaagagtaccccttccactaagcaaagggaagatgaacactcttctgaactattaaagaaaaatagagcaaatatctgtggacagaatcctggccctcttatacctcagccttcgagtcaacagactcctgagaggaaaccccgcttatctgaacactgccggctgtactttggggcgctcttcaaagccatcagcgcatttcacggcagcctcagcagcagccagcccgcggagatcatcactcagagcaagctggtcatcatggtgggacagaagctggtggacacgctgtgcatggagacccaggagagggacgtgcgcaatgagatcctccgcggcagcagtcacctctgcagcctgctcaaggacgtagcgctggccactaagaatgccgtgctcacataccccagccctgccgcgctggggcacctccaggcggaggctgagaagctggagcaacacacgcggcagttcagagggacactgggatgaggactgtctacctcccttcctcctctgctcacc

SEQ ID NO: 12 - DNA, 1160 bp, Homo sapiens
cctccgacagcctctccacaggtaccatgaaggtctccgcggcacgcctcgctgtcatcctcattgctactgccctctgcgctcctgcatctgcctccccatattcctcggacaccacaccctgctgctttgcctacattgcccgcccactgccccgtgcccacatcaaggagtatttctacaccagtggcaagtgctccaacccagcagtcgtctttgtcacccgaaagaaccgccaagtgtgtgccaacccagagaagaaatgggttcgggagtacatcaactctttggagatgagctaggatggagagtccttgaacctgaacttacacaaatttgcctgtttctgcttgctcttgtcctagcttgggaggcttcccctcactatcctaccccacccgctccttgaagggcccagattctgaccacgacgagcagcagttacaaaaaccttccccaggctggacgtggtggctcagccttgtaatcccagcactttgggaggccaaggtgggtggatcacttgaggtcaggagttcgagacagcctggccaacatgatgaaaccccatgtgtactaaaaatacaaaaaattagccgggcgtggtagcgggcgcctgtagtcccagctactcgggaggctgaggcaggagaatggcgtgaacccgggagcggagcttgcagtgagccgagatcgcgccactgcactccagcctgggcgacagagcgagactccgtctcaaaaaaaaaaaaaaaaaaaaaaaaaatacaaaaattagccgcgtggtggcccacgcctgtaatcccagctactcgggaggctaaggcaggaaaattgtttgaacccaggaggtggaggctgcagtgagctgagattgtgccacttcactccagcctgggtgacaaagtgagactccgtcacaacaacaacaacaaaaagcttccccaactaaagcctagaagagcttctgaggcgctgctttgtcaaaaggaagtctctaggttctgagctctggctttgccttggctttgcaagggctctgtgacaaggaaggaagtcagcatgcctctagaggcaaggaagggaggaacactgcactcttaagcttccgccgtctcaacccctcacaggagcttactggcaaacatgaaaaatcgggg

SEQ ID NO: 13 - DNA, 4658 bp, Homo sapiens
ggaattccgggaggagcggacgtcggcttctccccgcgggagcccccagcatggttgactatattgtggagtatgactatgatgctgtacatgatgatgaattaactattcgagttggagaaatcatcaggaatgtgaaaaagctacaggaggaagggtggctggaaggagaactaaatgggagaagaggaatgttccctgacaatttcgttaaggaaattaaaagagagacggaattcaaggatgacagtttgcccatcaaacgggaaaggcatgggaatgtagcaagtcttgtacaacgaataagcacctatggacttccagctggaggaattcagccacatccacaaaccaaaaacattaagaagaagaccaagaagcgtcagtgtaaagttctttttgagtacattccacaaaatgaggatgaactggagctgaaagtgggagatattattgatattaatgaagaggtagaagaaggctggtggagtggaaccctgaataacaagttgggactgtttccctcaaattttgtgaaagaattagaggtaacagatgatggtgaaactcatgaagcccaggacgattcagaaactgttttggctgggcctacttcacctataccttctctgggaaatgtgagtgaaactgcatctggatcagttacacagccaaagaaaattcgaggaattggatttggagacatttttaaagaaggttctgtgaaacttcggacaagaacatccagtagtgaaacagaagagaaaaaaccagaaaagcccttaatcctacagtcactgggacccaaaactcagagtgtggagataacaaaaacagataccgaaggtaaaattaaagctaaagaatattgtagaacattatttgcctatgaaggtactaatgaagatgaacttacttttaaagagggggagataatccatttgataagtaaggagactggagaagctggctggtggaggggcgaacttaatggtaaagaaggagtatttccagacaattttgctgtccagataaatgaacttgataaagactttccaaaaccaaagaaaccaccacctcctgctaaggctccagctccaaagcctgaactgatagctgcagagaagaaatatttttctttaaagcctgaagaaaaggatgaaaaatcaacactggaacagaaaccttctaaaccagcagctccacaagtcccacccaagaaacctactccacctaccaaagccagtaatttattgagatcttctggaacagtgtacccaaagcgacctgaaaaaccagttcctccaccacctcctatagccaagattaatggggaagtttctagcatttcatcaaaatttgaaactgagccagtatcaaaactaaagctagattctgaacagctgccccttagaccaaaatcagtagactttgattcacttacagtaaggacctccaaagaaacagatgttgtaaattttgatgacatagcttcctcagaaaacttgcttcatctcactgcaaatagaccaaagatgcctggaagaaggttgccgggccgtttcaatggtggacattctccaactcacagccccgaaaaaatcttgaagttaccaaaagaagaagacagtgccaacctgaagccatctgaattaaaaaaagatacatgctactctccaaagccatctgtgtacctttcaacaccttccagtgcttctaaagcaaatacaactgctttcctgactccattagaaatcaaagctaaagtggaaacagatgatgtgaaaaaaaattccctggatgaacttagagcccagattattgaattgttgtgcattgtagaagcactgaaaaaggatcacgggaaagaactggaaaaactgcgaaaagatttggaagaagagaagacaatgagaagtaatctagagatggaaatagagaagctgaaaaaagctgtcctgtcttcttgagtggtgtggacctggtgttcataatgttccagggattcagaagcaacgctatgaacttcagctgacttgttacttaaaaattgtgaattctgttgttgtgataaatatgagcaaatgaagtgtaatatctatagaaaagtagagtgagggtgaatttatatatatattttgttttgccaatatgaagaaaaagaggccttatttcttaactgtgctgggattgcaaacactttttaaaaaattgtttgcttgaaaatactactgaatataaataagaatgtgctcagtagtttttttattgaaacttgtattatttttaaagagatctatactataaatatggtgatatatttacaagtaatctgtaagatatactatttgagagggacagattagccttttagtaactatagtcactactttttccataatgcataagggatataaactctgtgtgtgtgtgtgtgtgtgtgtgtgtgtgtgtgtgtgtatatatatatatatatttttacttttatcctcttaccgaaggttacactgttgtgcctgtttgtctgcaatgctgtttatattttgggtgatgaaataggagtttcctagctatataaaccagattactcacccatgcatatagtaagaactaatgaataatcaaaataatttcatcaacttttagaatattttatgttgcttgcactataggagtcataaaaggaacttagttaaaatatgttggattgttaaacatttggggaaatatgaactgtattttaaatttgttaggtctgaaaaatctaaaactgttaatttaacccttaacttgtgcctagaaactacagcacatataaaatatgtaaacaccagcctgttgctgtacttttctgcttattttacagcctcaaatatttctcattatcttgtcacttagttcttcatgtttctccttctgacttttaataatggtaataggaaaacaaaacccaaagcttttcaaacttcagtgtgaggtttcctattttgacaagttaacttgtaaatactcaggttttacgatgtataatttacctaatagaccaaactaactcatggagatattttgaactattatttaggtacaaactttataaagaatgttagtatgtcataaaatataacattacagcttatttaaaaccaaatatattgaacatattttaaaatacatttcacagaatggatgaattagttgtttcttcaaaagttacttatgaacagttgaatgcctttaaaatgttctgtctgtaggtacatctaaaaacacaagtgggtttatttaaatttttaaaatttgaaattttttatttgccaaaaattgttttatgctttattatatcgcaaatgagtgtcagatttttgagtaccaatgatcatgcttccattttttttagttttaaaccaccaaaccaatatttttcctttaaattttaatcttataatatagaaatcttatgttaatgaaattttgtcatgtttcaaataaagaaaactgaagtagaaaatagaaatgccagtaaacaacataatgtttaatttacaacttacattaggggtttgggggaatgctaattatatattgagaatatacattagaactcttcaaaatgggctcttctaatgaggtcactactgaacaaaattgttccctcttctgttaaatagaataggtttaaatgactagtcaaatgaattattttctccttgttaaataaattaaatcttactttcttttaatgaccaaccttaggtaaaacaaaaatattgtaatcctagaaattatcctccagctttctcacctgaaaatctattgaagtgatccctggtcatcctaataatgggatgagggaagtttccagcagatttcaggctgttcttaaagtttttgttggtcattttctcaatagtacatgaaatcaagatgcttatgagcatggaaatgtatttaaagtttttgcttgtgtcctcctcagtcagaatagaaaagtaactgaaatactcttacctttctgtccttgataaaatagtaaagaaaaccaaacaaacccaggcctgatgggaaaaatgattcctttattctagcaattactttctgttggtatgggaaatgttattaatttctattactaaagttcatatcacaaaatgatatttaataataaccttggggtaaatcatgaatttttttttctacgtgtgagtataaaagacaaaagttgaacagcatggaatcttcattgccaaattattagtgaatgtatagttcaggtattctttgagacacacagtatcattaatttccgaattgtatttcagtgttattttttgtttgtgaccactaagcttctgtcttaatacaaagctgttaccttctacagaatttaagtctgaagatgtaaagagagaacaggccttgtgtaacagaagatactcttttttatgctccttactgtgatcacagaaaaattaaaaatccaagtgctctctagatttgttgataaacattttatgcttgcatttaaacttgaaatgtatgagcagaatgagacaatcagttaaatcagaaatgagaagtattataatgtaaaggccttgttttgctgtagcaataaaatgaccaagtgcaatgacttgatttaataaaatccggaattc

SEQ ID NO: 14 - DNA, 1437 bp, Homo sapiens
gcttcctcagacatgccgctgctgctactgctgcccctgctgtgggcaggggccctggctatggatccaaatttctggctgcaagtgcaggagtcagtgacggtacaggagggtttgtgcgtcctcgtgccctgcactttcttccatcccataccctactacgacaagaactccccagttcatggttactggttccgggaaggagccattatatccggggactctccagtggccacaaacaagctagatcaagaagtacaggaggagactcagggcagattccgcctccttggggatcccagtaggaacaactgctccctgagcatcgtagacgccaggaggagggataatggttcatacttctttcggatggagagaggaagtaccaaatacagttacaaatctccccagctctctgtgcatgtgacagacttgacccacaggcccaaaatcctcatccctggcactctagaacccggccactccaaaaaccttacctgctctgtgtcctgggcctgtgagcagggaacacccccgatcttctcctggttgtcagctgcccccacctccctgggccccaggactactcactcctcggtgctcataatcaccccacggccccaggaccacggcaccaacctgacctgtcaggtgaagttcgctggagctggtgtgactacggagagaaccatccagctcaacgtcacctatgttccacagaacccaacaactggtatctttccaggagatggctcagggaaacaagagaccagagcaggactggttcatggggccattggaggagctggtgttacagccctgctcgctctttgtctctgcctcatcttcttcatagtgaagacccacaggaggaaagcagccaggacagcagtgggcagcaatgacacccaccctaccacagggtcagcctccccgaaacaccagaagaactccaagttacatggccccactgaaacctcaagctgttcaggtgccgcccctactgtggagatggatgaggagctgcattatgcttccctcaactttcatgggatgaatccttccaaggacacctccaccgaatactcagaggtcaggacccagtgaggaaccctcaagagcatcaggctcagctagaagatccacatcctctacaggtcggggaccaaaggctgattcttggagatttaactccccacaggcaatgggtttatagacattatgtgagtttcctgctatattaacatcatcttgagactttgcaagcagagagtcgtggaatcaaatctgtgctctttcatttgctaagtgtatgatgtcacacaagctccttaaccttccatgtctccattttcttctctgtgaagtaggtataagaagtcctatctcatagggatgctgtgagcattaaataaaggtacacatggaaaacaccag

SEQ ID NO: 15 - DNA, 1872 bp, Homo sapiens
ttaattacaaaaactaatgactaagagagaggtggctagagctgaggcccctgagtcaggctgtgggtgggatcatctccagtacaggaagtgagactttcatttcctcctttccaagagagggctgagggagcagggttgagcaactggtgcagacagcctagctggactttgggtgaggcggttcagccatgaggctggctgtgcttttctcgggggccctgctggggctactggcagcccaggggacagggaatgactgtcctcacaaaaaatcagctactttgctgccatccttcacggtgacacccacggttacagagagcactggaacaaccagccacaggactaccaagagccacaaaaccaccactcacaggacaaccaccacaggcaccaccagccacggacccacgactgccactcacaaccccaccaccaccagccatggaaacgtcacagttcatccaacaagcaatagcactgccaccagccagggaccctcaactgccactcacagtcctgccaccactagtcatggaaatgccacggttcatccaacaagcaacagcactgccaccagcccaggattcaccagttctgcccacccagaaccacctccaccctctccgagtcctagcccaacctccaaggagaccattggagactacacgtggaccaatggttcccagccctgtgtccacctccaagcccagattcagattcgagtcatgtacacaacccagggtggaggagaggcctggggcatctctgtactgaaccccaacaaaaccaaggtccagggaagctgtgagggtgcccatccccacctgcttctctcattcccctatggacacctcagctttggattcatgcaggacctccagcagaaggttgtctacctgagctacatggcggtggagtacaatgtgtccttcccccacgcagcacagtggacattctcggctcagaatgcatcccttcgagatctccaagcacccctggggcagagcttcagttgcagcaactcgagcatcattctttcaccagctgtccacctcgacctgctctccctgaggctccaggctgctcagctgccccacacaggggtctttgggcaaagtttctcctgccccagtgaccggtccatcttgctgcctctcatcatcggcctgatccttcttggcctcctcgccctggtgcttattgctttctgcatcatccggagacgcccatccgcctaccaggccctctgagcatttgcttcaaaccccagggcactgagggggttggggtgtggtgggggggtacccttatttcctcgacacgcaactggctcaaagacaatgttattttccttccctttcttgaagaacaaaaagaaagccgggcatgacggctcatgcctgtaatcccagcactttgggaggctgaggcaggtggatcactggaggtcaggagtttgagaccagcctggccaacatggtgaaaccctgtctctactaaaaatacaattagccaggtgtggcggcgtaatcccagctggcctgtaatcccagctacttgggaggctgaggcagaactgcttgaacccaggaggtggaggttgcagtgagccgtcatcgcgccactaagccaagatcgcgccactgcactccagcctgggcgacagagccagactgtctcaaataaataaatatgagataatgcagtcgggagaagggagggagagaattttattaaatgtgacgaactgccccccccccccccccagcaggagagcagcaaaatttatgcaaatctttgacggggttttccttgtcctgccaggattaaaagccatgagtttcttgtcaaaaaaaaaaaaaaaaa

SEQ ID NO: 16 - DNA, 2649 bp, Homo sapiens
aggttggtccttccatagccggaagtggccttcctgagaggcgtggctgcggcactcttgccggatagggtggcccggcggggctaggaaagcgtgaaatctcgcgcgattgcgctgcgaagtcggggacggggcggggctggcggcgggggcggggacccggagcgggaagatggcggcggcgcaggaggcggacggggcccgcagcgccgtggtggcggccgggggaggcagctccggtcaggtgaccagcaatggcagcatcgggagggacccgccagcggagacccagcctcagaacccaccggcccagccggcacccaatgcctggcaggtcatcaaaggtgtgctgtttaggatcttcatcatctgggccatcagcagttggttccgccgagggccggcccctcaggaccaggcgggccccggaggagctccacgcgtcgccagccgcaacctgttccccaaagacactttaatgaacctgcatgtgtacatctcagagcacgagcactttacagacttcaacgccacgtcggcactcttctgggaacagcacgatcttgtgtatggcgactggactagcggcgagaactcagacggctgctacgagcactttgctgagctcgatatcccacagagcgtccagcagaacggctccatctacatccacgtttacttcaccaagagtggcttccacccagacccccggcagaaggccctgtaccgccggcttgccacagtccacatgtcccggatgatcaacaaatacaagcgcagacgatttcagaaaaccaagaacctgctgacaggagagacagaagcggacccagaaatgatcaagagggctgaggactatgggcctgtggaggtgatctcccattggcaccccaacatcaccatcaacatcgtggacgaccacacgccgtgggtgaagggcagtgtgccccctcccctggatcaatatgtgaagttcgacgccgtgagcggtgactactatcccatcatctacttcaatgactactggaacctgcagaaggactactaccccatcaacgagagcctggccagcctgccgctccgcgtctccttctgcccactctcgctttggcgctggcagctctatgctgcccagagcaccaagtcgccctggaacttcctgggtgatgagttgtacgagcagtcagatgaggagcaggactcggtgaaggtggccctgctggagaccaacccctacctgctggcgctcaccatcatcgtgtctatcgttcacagtgtcttcgagttcctggccttcaagaatgatatccagttctggaacagccggcagtccctggagggcctgtccgtgcgctccgtcttcttcggcgttttccagtcattcgtggtcctcctctacatcctggacaacgagaccaacttcgtggtccaggtcagcgtcttcattggggtcctcatcgacctctggaagatcaccaaggtcatggacgtccggctggaccgagagcacagggtggcaggaatcttcccccgcctatccttcaaggacaagtccacgtatatcgagtcctcgaccaaagtgtatgatgatatggcattccggtacctgtcctggatcctcttcccgctcctgggctgctatgccgtctacagtcttctgtacctggagcacaagggctggtactcctgggtgctcagcatgctctacggcttcctgctgaccttcggcttcatcaccatgacgccccagctcttcatcaactacaagctcaagtctgtggcccaccttccctggcgcatgctcacctacaaggccctcaacacattcatcgacgacctgttcgcctttgtcatcaagatgcccgttatgtaccggatcggctgcctgcgggacgatgtggttttcttcatctacctctaccaacggtggatctaccgcgtcgaccccacccgagtcaacgagtttggcatgagtggagaagaccccacagctgccgcccccgtggccgaggttcccacagcagcaggggccctcacgcccacacctgcacccaccacgaccaccgccaccagggaggaggcctccacgtccctgcccaccaagcccacccagggggccagctctgccagcgagccccaggaagcccctccaaagccagcagaggacaagaaaaaggattagtcgagactggtcctcacctgctccggctcctggcgaccactacccctgcgtcccggccccctcgcctcccctccctgtcgccctttccctggacagatcaggccggggcggtgggaggcccgcctcaggtcagggcccagcgtgtgatgtaggggccggggcaggccagggtttgtttgtggaggcgctgtctgtccctctgtccctctgtgtttccagccatctcgccctgccagcccagcaccactgggaatcatggtgaagctgatgcagcgttgccgagggggtgggttgggcgggggtggggccgggcccccctacgggatgcccacggccgttcatcatcttgtccctcgtccccctaccacactccccctcctagaccgccgccctttaacacagtctggatttaataaattcatatgggtgtttaacttaaactcagcactaaaaaaaaaaaaaaaaaa

SEQ ID NO: 17 - DNA, 1651 bp, Homo sapiens
ctgcgaaccctctctactctccgaagggaattgtccttcctggcttccactacttccacccctgaatgcacaggcagcccggcccaagtctcccactagggatgcagatggattcggtgtgaagggctggctgctgttgcctccggctcttgaaagtcaagttcagaggcgtgcaaagactccagaattggaggcatgatgaagactctgctgctgtttgtggggctgctgctgacctgggagagtgggcaggtcctgggggaccagacggtctcagacaatgagctccaggaaatgtccaatcagggaagtaagtacgtcaataaggaaattcaaaatgctgtcaacggggtgaaacagataaagactctcatagaaaaaacaaacgaagagcgcaagacactgctcagcaacctagaagaagccaagaagaagaaagaggatgccctaaatgagaccagggaatcagagacaaagctgaaggagctcccaggagtgtgcaatgagaccatgatggccctctgggaagagtgtaagccctgcctgaaacagacctgcatgaagttctacgcacgcgtctgcagaagtggctcaggcctggttggccgccagcttgaggagttcctgaaccagagctcgcccttctacttctggatgaatggtgaccgcatcgactccctgctggagaacgaccggcagcagacgcacatgctggatgtcatgcaggaccacttcagccgcgcgtccagcatcatagacgagctcttccaggacaggttcttcacccgggagccccaggatacctaccactacctgcccttcagcctgccccaccggaggcctcacttcttctttcccaagtcccgcatcgtccgcagcttgatgcccttctctccgtacgagcccctgaacttccacgccatgttccagcccttccttgagatgatacacgaggctcagcaggccatggacatccacttccacagcccggccttccagcacccgccaacagaattcatacgagaaggcgacgatgaccggactgtgtgccgggagatccgccacaactccacgggctgcctgcggatgaaggaccagtgtgacaagtgccgggagatcttgtctgtggactgttccaccaacaacccctcccaggctaagctgcggcgggagctcgacgaatccctccaggtcgctgagaggttgaccaggaaatataacgagctgctaaagtcctaccagtggaagatgctcaacacctcctccttgctggagcagctgaacgagcagtttaactgggtgtcccggctggcaaacctcacgcaaggcgaagaccagtactatctgcgggtcaccacggtggcttcccacacttctgactcggacgttccttccggtgtcactgaggtggtcgtgaagctctttgactctgatcccatcactgtgacggtccctgtagaagtctccaggaagaaccctaaatttatggagaccgtggcggagaaagcgctgcaggaataccgcaaaaagcaccgggaggagtgagatgtggatgttgcttttgcacctacgggggcatctgagtccagctccccccaagatgagctgcagccccccagagagagctctgcacgtcaccaagtaaccaggc

SEQ ID NO: 18 - DNA, 6951 bp, Homo sapiens
cgtggtttgtagatgtgcttggggagaatgggggcctcttctccaagaagcccggagcctgtcgggccgccggcgcccggtctccccttctgctgcggaggatccctgctggcggttgtggtgctgcttgcgctgccggtggcctggggtcaatgcaatgccccagaatggcttccatttgccaggcctaccaacctaactgatgagtttgagtttcccattgggacatatctgaactatgaatgccgccctggttattccggaagaccgttttctatcatctgcctaaaaaactcagtctggactggtgctaaggacaggtgcagacgtaaatcatgtcgtaatcctccagatcctgtgaatggcatggtgcatgtgatcaaaggcatccagttcggatcccaaattaaatattcttgtactaaaggataccgactcattggttcctcgtctgccacatgcatcatctcaggtgatactgtcatttgggataatgaaacacctatttgtgacagaattccttgtgggctaccccccaccatcaccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggccccgcccctcagtgcattatacctaacaaatgcacgcctccaaatgtggaaaatggaatattggtatctgacaacagaagcttattttccttaaatgaagttgtggagtttaggtgtcagcctggctttgtcatgaaaggaccccgccgtgtgaagtgccaggccctgaacaaatgggagccggagctaccaagctgctccagggtatgtcagccacctccagatgtcctgcatgctgagcgtacccaaagggacaaggacaacttttcacctgggcaggaagtgttctacagctgtgagcccggctacgacctcagaggggctgcgtctatgcgctgcacaccccagggagactggagccctgcagcccccacatgtgaagtgaaatcctgtgatgacttcatgggccaacttcttaatggccgtgtgctatttccagtaaatctccagcttggagcaaaagtggattttgtttgtgatgaaggatttcaattaaaaggcagctctgctagttactgtgtcttggctggaatggaaagcctttggaatagcagtgttccagtgtgtgaacaaatcttttgtccaagtcctccagttattcctaatgggagacacacaggaaaacctctggaagtctttccctttggaaaagcagtaaattacacatgcgacccccacccagacagagggacgagcttcgacctcattggagagagcaccatccgctgcacaagtgaccctcaagggaatggggtttggagcagccctgcccctcgctgtggaattctgggtcactgtcaagccccagatcattttctgtttgccaagttgaaaacccaaaccaatgcatctgactttcccattgggacatctttaaagtacgaatgccgtcctgagtactacgggaggccattctctatcacatgtctagataacctggtctggtcaagtcccaaagatgtctgtaaacgtaaatcatgtaaaactcctccagatccagtgaatggcatggtgcatgtgatcacagacatccaggttggatccagaatcaactattcttgtactacagggcaccgactcattggtcactcatctgctgaatgtatcctctcgggcaatgctgcccattggagcacgaagccgccaatttgtcaacgaattccttgtgggctaccccccaccatcgccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggcccggcccctcagtgcattatacctaacaaatgcacgcctccaaatgtggaaaatggaatattggtatctgacaacagaagcttattttccttaaatgaagttgtggagtttaggtgtcagcctggctttgtcatgaaaggaccccgccgtgtgaagtgccaggccctgaacaaatgggagccggagctaccaagctgctccagggtatgtcagccacctccagatgtcctgcatgctgagcgtacccaaagggacaaggacaacttttcacccgggcaggaagtgttctacagctgtgagcccggctatgacctcagaggggctgcgtctatgcgctgcacaccccagggagactggagccctgcagcccccacatgtgaagtgaaatcctgtgatgacttcatgggccaacttcttaatggccgtgtgctatttccagtaaatctccagcttggagcaaaagtggattttgtttgtgatgaaggatttcaattaaaaggcagctctgctagttattgtgtcttggctggaatggaaagcctttggaatagcagtgttccagtgtgtgaacaaatcttttgtccaagtcctccagttattcctaatgggagacacacaggaaaacctctggaagtctttccctttggaaaagcagtaaattacacatgcgacccccacccagacagagggacgagcttcgacctcattggagagagcaccatccgctgcacaagtgaccctcaagggaatggggtttggagcagccctgcccctcgctgtggaattctgggtcactgtcaagccccagatcattttctgtttgccaagttgaaaacccaaaccaatgcatctgactttcccattgggacatctttaaagtacgaatgccgtcctgagtactacgggaggccattctctatcacatgtctagataacctggtctggtcaagtcccaaagatgtctgtaaacgtaaatcatgtaaaactcctccagatccagtgaatggcatggtgcatgtgatcacagacatccaggttggatccagaatcaactattcttgtactacagggcaccgactcattggtcactcatctgctgaatgtatcctctcaggcaatactgcccattggagcacgaagccgccaatttgtcaacgaattccttgtgggctacccccaaccatcgccaatggagatttcattagcaccaacagagagaattttcactatggatcagtggtgacctaccgctgcaatcttggaagcagagggagaaaggtgtttgagcttgtgggtgagccctccatatactgcaccagcaatgacgatcaagtgggcatctggagcggccccgcccctcagtgcattatacctaacaaatgcacgcctccaaatgtggaaaatggaatattggtatctgacaacagaagcttattttccttaaatgaagttgtggagtttaggtgtcagcctggctttgtcatgaaaggaccccgccgtgtgaagtgccaggccctgaacaaatgggagccagagttaccaagctgctccagggtgtgtcagccgcctccagaaatcctgcatggtgagcataccccaagccatcaggacaacttttcacctgggcaggaagtgttctacagctgtgagcctggctatgacctcagaggggctgcgtctctgcactgcacaccccagggagactggagccctgaagccccgagatgtgcagtgaaatcctgtgatgacttcttgggtcaactccctcatggccgtgtgctatttccacttaatctccagcttggggcaaaggtgtcctttgtctgtgatgaagggtttcgcttaaagggcagttccgttagtcattgtgtcttggttggaatgagaagcctttggaataacagtgttcctgtgtgtgaacatatcttttgtccaaatcctccagctatccttaatgggagacacacaggaactccctctggagatattccctatggaaaagaaatatcttacacatgtgacccccacccagacagagggatgaccttcaacctcattggggagagcaccatccgctgcacaagtgaccctcatgggaatggggtttggagcagccctgcccctcgctgtgaactttctgttcgtgctggtcactgtaaaaccccagagcagtttccatttgccagtcctacgatcccaattaatgactttgagtttccagtcgggacatctttgaattatgaatgccgtcctgggtattttgggaaaatgttctctatctcctgcctagaaaacttggtctggtcaagtgttgaagacaactgtagacgaaaatcatgtggacctccaccagaacccttcaatggaatggtgcatataaacacagatacacagtttggatcaacagttaattattcttgtaatgaagggtttcgactcattggttccccatctactacttgtctcgtctcaggcaataatgtcacatgggataagaaggcacctatttgtgagatcatatcttgtgagccacctccaaccatatccaatggagacttctacagcaacaatagaacatcttttcacaatggaacggtggtaacttaccagtgccacactggaccagatggagaacagctgtttgagcttgtgggagaacggtcaatatattgcaccagcaaagatgatcaagttggtgtttggagcagccctccccctcggtgtatttctactaataaatgcacagctccagaagttgaaaatgcaattagagtaccaggaaacaggagtttcttttccctcactgagatcatcagatttagatgtcagcccgggtttgtcatggtagggtcccacactgtgcagtgccagaccaatggcagatgggggcccaagctgccacactgctccagggtgtgtcagccgcctccagaaatcctgcatggtgagcataccctaagccatcaggacaacttttcacctgggcaggaagtgttctacagctgtgagcccagctatgacctcagaggggctgcgtctctgcactgcacgccccagggagactggagccctgaagcccctagatgtacagtgaaatcctgtgatgacttcctgggccaactccctcatggccgtgtgctacttccacttaatctccagcttggggcaaaggtgtcctttgtttgcgatgaagggttccgattaaaaggcaggtctgctagtcattgtgtcttggctggaatgaaagccctttggaatagcagtgttccagtgtgtgaacaaatcttttgtccaaatcctccagctatccttaatgggagacacacaggaactccctttggagatattccctatggaaaagaaatatcttacgcatgcgacacccacccagacagagggatgaccttcaacctcattggggagagctccatccgctgcacaagtgaccctcaagggaatggggtttggagcagccctgcccctcgctgtgaactttctgttcctgctgcctgcccacatccacccaagatccaaaacgggcattacattggaggacacgtatctctatatcttcctgggatgacaatcagctacacttgtgaccccggctacctgttagtgggaaagggcttcattttctgtacagaccagggaatctggagccaattggatcattattgcaaagaagtaaattgtagcttcccactgtttatgaatggaatctcgaaggagttagaaatgaaaaaagtatatcactatggagattatgtgactttgaagtgtgaagatgggtatactctggaaggcagtccctggagccagtgccaggcggatgacagatgggaccctcctctggccaaatgtacctctcgtgcacatgatgctctcatagttggcactttatctggtacgatcttctttattttactcatcattttcctctcttggataattctaaagcacagaaaaggcaataatgcacatgaaaaccctaaagaagtggctatccatttacattctcaaggaggcagcagcgttcatccccgaactctgcaaacaaatgaagaaaatagcagggtccttccttgacaaagtactatacagctgaagaacatctcgaatacaattttggtgggaaaggagccaattgatttcaacagaatcagatctgagcttcataaagtctttgaagtgacttcacagagacgcagacatgtgcacttgaagatgctgccccttccctggtacctagcaaagctcctgcctctttgtgtgcgtcactgtgaaacccccacccttctgcctcgtgctaaacgcacacagtatctagtcaggggaaaagactgcatttaggagatagaaaatagtttggattacttaaaggaataaggtgttgcctggaatttctggtttgtaaggtggtcactgttcttttttaaaatatttgtaatatggaatgggctcagtaagaagagcttggaaaatgcagaaagttatgaaaaataagtcacttataattatgctacctactgataaccactcctaatattttgattcattttctgcctatcttctttcacatatgtgtttttttacatacgtacttttccccccttagtttgtttccttttattttatagagcagaaccctagtcttttaaacagtttagagtgaaatatatgctatatcagtttttactttctctagggagaaaaattaatttactagaaaggcatgaaatgatcatgggaagagtggttaagactactgaagagaaatatttggaaaataagatttcgatatcttctttttttttgagatggagtctggctctgtctcccaggctggagtgcagtggcgtaatctcggctcactgcaacgtccgcctcccg

SEQ ID NO: 19 - DNA, 3939 bp, Homo sapiens
cctgggtcctctcggcgccagagccgctctccgcatcccaggacagcggtgcggccctcggccggggcgcccactccgcagcagccagcgagccagctgccccgtatgaccgcgccgggcgccgccgggcgctgccctcccacgacatggctgggctccctgctgttgttggtctgtctcctggcgagcaggagtatcaccgaggaggtgtcggagtactgtagccacatgattgggagtggacacctgcagtctctgcagcggctgattgacagtcagatggagacctcgtgccaaattacatttgagtttgtagaccaggaacagttgaaagatccagtgtgctaccttaagaaggcatttctcctggtacaagacataatggaggacaccatgcgcttcagagataacaccgccaatcccatcgccattgtgcagctgcaggaactctctttgaggctgaagagctgcttcaccaaggattatgaagagcatgacaaggcctgcgtccgaactttctatgagacacctctccagttgctggagaaggtcaagaatgtctttaatgaaacaaagaatctccttgacaaggactggaatattttcagcaagaactgcaacaacagctttgctgaatgctccagccaagatgtggtgaccaagcctgattgcaactgcctgtaccccaaagccatccctagcagtgacccggcctctgtctcccctcatcagcccctcgccccctccatggcccctgtggctggcttgacctgggaggactctgagggaactgagggcagctccctcttgcctggtgagcagcccctgcacacagtggatccaggcagtgccaagcagcggccacccaggagcacctgccagagctttgagccgccagagaccccagttgtcaaggacagcaccatcggtggctcaccacagcctcgcccctctgtcggggccttcaaccccgggatggaggatattcttgactctgcaatgggcactaattgggtcccagaagaagcctctggagaggccagtgagattcccgtaccccaagggacagagctttccccctccaggccaggagggggcagcatgcagacagagcccgccagacccagcaacttcctctcagcatcttctccactccctgcatcagcaaagggccaacagccggcagatgtaactgctacagccttgcccagggtgggccccgtgatgcccactggccaggactggaatcacaccccccagaagacagaccatccatctgccctgctcagagaccccccggagccaggctctcccaggatctcatcactgcgcccccaggccctcagcaacccctccaccctctctgctcagccacagctttccagaagccactcctcgggcagcgtgctgccccttggggagctggagggcaggaggagcaccagggatcggacgagccccgcagagccagaagcagcaccagcaagtgaaggggcagccaggcccctgccccgttttaactccgttcctttgactgacacaggccatgagaggcagtccgagggatcctccagcccgcagctccaggagtctgtcttccacctgctggtgcccagtgtcatcctggtcttgctggctgtcggaggcctcttgttctacaggtggaggcggcggagccatcaagagcctcagagagcggattctcccttggagcaaccagagggcagccccctgactcaggatgacagacaggtggaactgccagtgtagagggaattctaagctggacgcacagaacagtctcttcgtgggaggagacattatggggcgtccaccaccacccctccctggccatcctcctggaatgtggtctgccctccaccagagctcctgcctgccaggactggaccagagcagccaggctggggcccctctgtctcaacccgcagacccttgactgaatgagagaggccagaggatgctccccatgctgccactatttattgtgagccctggaggctcccatgtgcttgaggaaggctggtgagcccggctcaggaccctcttccctcaggggctgcagcctcctctcactcccttccatgccggaacccaggccagggacccaccggcctgtggtttgtgggaaagcagggtgcacgctgaggagtgaaacaaccctgcacccagagggcctgcctggtgccaaggtatcccagcctggacaggcatggacctgtctccagacagaggagcctgaagttcgtggggcgggacagcctcggcctgatttcccgtaaaggtgtgcagcctgagagacgggaagaggaggcctctgcacctgctggtctgcactgacagcctgaagggtctacaccctcggctcacctaagtccctgtgctggttgccaggcccagaggggaggccagccctgccctcaggacctgcctgacctgccagtgatgccaagagggggatcaagcactggcctctgcccctcctccttccagcacctgccagagcttctccagcaggccaagcagaggctcccctcatgaaggaagccattgcactgtgaacactgtacctgcctgctgaacagcctccccccgtccatccatgagccagcatccgtccgtcctccactctccagcctctccccagcctcctgcactgagctggcctcaccagtcgactgagggagcccctcagccctgaccttctcctgacctggcctttgactccccggagtggagtggggtgggagaacctcctgggccgccagccagagccgctctttaggctgtgttcttcgcccaggtttctgcatcttccactttgacattcccaagagggaagggactagtgggagagagcaagggaggggagggcacagacagagagcctacagggcgagctctgactgaagatgggcctttgaaatataggtatgcacctgaggttgggggagggtctgcactcccaaaccccagcgcagtgtcctttccctgctgccgacaggaacctggggctgagcaggttatccctgtcaggagccctggactgggctgcatctcagccccacctgcatggtatccagctcccatccacttctcacccttctttcctcctgaccttggtcagcagtgatgacctccaactctcacccaccccctctaccatcacctctaaccaggcaagccagggtgggagagcaatcaggagagccaggcctcagcttccaatgcctggagggcctccactttgtggccagcctgtggtgctggctctgaggcctaggcaacgagcgacagggctgccagttgcccctgggttcctttgtgctgctgtgtgcctcctctcctgccgccctttgtcctccgctaagagaccctgccctacctggccgctgggccccgtgactttcccttcctgcccaggaaagtgagggtcggctggccccaccttccctgtcctgatgccgacagcttagggaagggcactgaacttgcatatggggcttagccttctagtcacagcctctatatttgatgctagaaaacacatatttttaaatggaagaaaaataaaaaggcattcccccttcatccccctaccttaaacatataatattttaaaggtcaaaaaagcaatccaacccactgcagaagctctttttgagcacttggtggcatcagagcaggaggagccccagagccacctctggtgtcccccaggctacctgctcaggaaccccttctgttctctgagaactcaacagaggacattggctcacgcactgtgagattttgtttttatacttgcaactggtgaattattttttataaagtcatttaaatatctatttaaaagataggaagctgcttatatatttaataataaaagaagtgcacaagctgccgttgacgtagctcgag

SEQ ID NO: 20 - DNA, 867 bp, Homo sapiens
ggctcagcacaggcacaaaccattgcccggcactggcccgtgctgcctgagaaggattggcacgggcacagaccactgcccccacctgccctgcgccatctacccaagaaggctcggcacgggcaccaaccactgcctccaactgccccatgctgcctgagaaggcactgcacggccacccccaactgccccgcactgtccctacccgggcagccatgcgagcggctggaactctgctggccttctgctgcctggtcttgagcaccactgggggcccttccccagatacttgttcccaggaccttaactcacgtgtgaagccaggatttcctaaaacaataaagaccaatgacccaggagtcctccaagcagccagatacagtgttgaaaagttcaacaactgcacgaacgacatgttcttgttcaaggagtcccgcatcacaagggccctagttcagatagtgaaaggcctgaaatatatgctggaggtggaaattggcagaactacctgcaagaaaaaccagcacctgcgtctggatgactgtgacttccaaaccaaccacaccttgaagcagactctgagctgctactctgaagtctgggtcgtgccctggctccagcacttcgaggtgcctgttctccgttgtcactgacccccgcctcttcagcaagaccacagccatgacaaacaccaggatgcatgctccttgtcccctcccacccgcctcatgacccagcctcacagaccctctcaggcctctgacgagtgagcgggtgaagtgccactgggtcaccgcagggcagctggaatggcagcatggtagcacctcctaacagattaaatagatcacatttgcttctaaaatt

SEQ ID NO: 21 - DNA, 2002 bp, Homo sapiens
aattccgcggcaaccgctccggcaacgccaaccgctccgctgcgcgcaggctgggctgcaggctctcggctgcagcgctgggctggtgtgcagtggtgcgaccacggctcacggcagcctcagccacccagatgtaagcgatctggttcccacctcagccttccgagtagtggatctaggatctggcttccaacatgtggcagctctgggcctccctctgctgcctgctggtgttggccaatgcccggagcaggccctctttccatcccgtgtcggatgagctggtcaactatgtcaacaaacggaataccacgtggcaggccgggcacaacttctacaacgtggacatgagctacttgaagaggctatgtggtaccttcctgggtgggcccaagccaccccagagagttatgtttaccgaggacctgaagctgcctgcaagcttcgatgcacgggaacaatggccacagtgtcccaccatcaaagagatcagagaccagggctcctgtggctcctgctgggccttcggggctgtggaagccatctctgaccgcatctgcatccacaccaatgcgcacgtcagcgtggaggtgtcggcggaggacctgctcacctgctgtggcagcatgtgtggggacggctgtaatggtggctatcctgctgaagcttggaacttctggacaagaaaaggcctggtttctggtggcctctatgaatcccatgtagggtgcagaccgtactccatccctccctgtgagcaccacgtcaacggctcccggcccccatgcacgggggagggagatacccccaagtgtagcaagatctgtgagcctggctacagcccgacctacaaacaggacaagcactacggatacaattcctacagcgtctccaatagcgagaaggacatcatggccgagatctacaaaaacggccccgtggagggagctttctctgtgtattcggacttcctgctctacaagtcaggagtgtaccaacacgtcaccggagagatgatgggtggccatgccatccgcatcctgggctggggagtggagaatggcacaccctactggctggttgccaactcctggaacactgactggggtgacaatggcttctttaaaatactcagaggacaggatcactgcggaatcgaatcagaagtggtggctggaattccacgcaccgatcagtactgggaaaagatctaatctgccgtgggcctgtcgtgccagtcctgggggcgagatcggggtagaaagtcattttattctttaagttcacgtaagatacaagtttcaggcagggtctgaaggactggattggccaaagtcctccaaggagaccaagtcctggctacatcccagcctgtggttacagtgcagacaggccatgtgagccaccgctgccagcacagagcgtccttccccctgtagactagtgccgtgggagtacctgctgcccagctgctgtggccccctccgtgatccatccatctccagggagcaagacagagacgcaggatggaaagcggagttcctaacaggatgaaagttcccccatcagttcccccagtacctccaagcaagtagctttccacatttgtcacagaaatcagaggagagatggtgttgggagccctttggagaacgccagtctccaggtccccctgcatctatcgagtttgcaatgtcacaacctctctgatcttgtgctcagcatgattctttaatagaagttttatttttcgtgcactctgctaatcatgtgggtgagccagtggaacagcgggagcctgtgctggtttgcagattgcctcctaatgacgcggctcaaaaggaaaccaagtggtcaggagttgtttctgacccactgatctctactaccacaaggaaaatagtttaggagaaaccagcttttactgtttttgaaaaattacagcttcaccctgtcaagttaacaaggaatgcctgtgccaataaaaggtttctccaacttg

SEQ ID NO: 22 - DNA, 2038 bp, Homo sapiens
ggctataagcgcacggcctcggcgaccctctccgacccggccgccgccgccatgcagccctccagccttctgccgctcgccctctgcctgctggctgcacccgcctccgcgctcgtcaggatcccgctgcacaagttcacgtccatccgccggaccatgtcggaggttgggggctctgtggaggacctgattgccaaaggccccgtctcaaagtactcccaggcggtgccagccgtgaccgaggggcccattcccgaggtgctcaagaactacatggacgcccagtactacggggagattggcatcgggacgcccccccagtgcttcacagtcgtcttcgacacgggctcctccaacctgtgggtcccctccatccactgcaaactgctggacatcgcttgctggatccaccacaagtacaacagcgacaagtccagcacctacgtgaagaatggtacctcgtttgacatccactatggctcgggcagcctctccgggtacctgagccaggacactgtgtcggtgccctgccagtcagcgtcgtcagcctctgccctgggcggtgtcaaagtggagaggcaggtctttggggaggccaccaagcagccaggcatcaccttcatcgcagccaagttcgatggcatcctgggcatggcctacccccgcatctccgtcaacaacgtgctgcccgtcttcgacaacctgatgcagcagaagctggtggaccagaacatcttctccttctacctgagcagggacccagatgcgcagcctgggggtgagctgatgctgggtggcacagactccaagtattacaagggttctctgtcctacctgaatgtcacccgcaaggcctactggcaggtccacctggaccaggtggaggtggccagcgggctgaccctgtgcaaggagggctgtgaggccattgtggacacaggcacttccctcatggtgggcccggtggatgaggtgcgcgagctgcagaaggccatcggggccgtgccgctgattcagggcgagtacatgatcccctgtgagaaggtgtccaccctgcccgcgatcacactgaagctgggaggcaaaggctacaagctgtccccagaggactacacgctcaaggtgtcgcaggccgggaagaccctctgcctgagcggcttcatgggcatggacatcccgccacccagcgggccactctggatcctgggcgacgtcttcatcggccgctactacactgtgtttgaccgtgacaacaacagggtgggcttcgccgaggctgcccgcctctagttcccaaggcgtccgcgcgccagcacagaaacagaggagagtcccagagcaggaggcccctggcccagcggcccctcccacacacacccacacactcgcccgcccactgtcctgggcgccctggaagccggcggcccaagcccgacttgctgttttgttctgtggttttcccctccctgggttcagaaatgctgcctgcctgtctgtctctccatctgtttggtgggggtagagctgatccagagcacagatctgtttcgtgcattggaagaccccacccaagcttggcagccgagctcgtgtatcctggggctcccttcatctccagggagtcccctccccggccctaccagcgcccgctgggctgagcccctaccccacaccaggccgtcctcccgggccctcccttggaaacctgccctgcctgagggcccctctgcccagcttgggcccagctgggctctgccaccctacctgttcagtgtcccgggcccgttgaggatgaggccgctagaggcctgaggatgagctggaaggagtgagaggggacaaaacccaccttgttggagcctgcagggtggtgctgggactgagccagtcccaggggcatgtattggcctggaggtggggttgggattgggggctggtgccagccttcctctgcagctgacctctgttgtcctccccttgggcggctgagagccccagctgacatggaaatacagttgttggcctccggcctcccctc

SEQ ID NO: 23 - DNA, 1575 bp, Homo sapiens
agaaccgcgacctccgcaaccttgagcggcatccgtggagtgcgcctgcagctacgaccgcagcaggaaagcgccgccggccaggcccagctgtggccggacagggactggaagagaggacgcggtcgagtaggtgtgcaccagccctggcaacgagagcgtctaccccgaactctgctggccttgaggtggggaagccggggagggcagttgaggaccccgcggaggcgcgtgactggttgagcgggcaggccagcctccgagccgggtggacacaggttttaaaacatgaatcctacactcatccttgctgccttttgcctgggaattgcctcagctactctaacatttgatcacagtttagaggcacagtggaccaagtggaaggcgatgcacaacagattatacggcatgaatgaagaaggatggaggagagcagtgtgggagaagaacatgaagatgattgaactgcacaatcaggaatacagggaagggaaacacagcttcacaatggccatgaacgcctttggagacatgaccagtgaagaattcaggcaggtgatgaatggctttcaaaaccgtaagcccaggaaggggaaagtgttccaggaacctctgttttatgaggcccccagatctgtggattggagagagaaaggctacgtgactcctgtgaagaatcagggtcagtgtggttcttgttgggcttttagtgctactggtgctcttgaaggacagatgttccggaaaactgggaggcttatctcactgagtgagcagaatctggtagactgctctgggcctcaaggcaatgaaggctgcaatggtggcctaatggattatgctttccagtatgttcaggataatggaggcctggactctgaggaatcctatccatatgaggcaacagaagaatcctgtaagtacaatcccaagtattctgttgctaatgacaccggctttgtggacatccctaagcaggagaaggccctgatgaaggcagttgcaactgtggggcccatttctgttgctattgatgcaggtcatgagtccttcctgttctataaagaaggcatttattttgagccagactgtagcagtgaagacatggatcatggtgtgctggtggttggctacggatttgaaagcacagaatcagataacaataaatattggctggtgaagaacagctggggtgaagaatggggcatgggtggctacgtaaagatggccaaagaccggagaaaccattgtggaattgcctcagcagccagctaccccactgtgtgagctggtggacggtgatgaggaaggacttgactggggatggcgcatgcatgggaggaattcatcttcagtctaccagcccccgctgtgtcggatacacactcgaatcattgaagatccgagtgtgatttgaattctgtgatattttcacactggtaaatgttacctctattttaattactgctataaataggtttatattattgattcacttactgactttgcattttcgtttttaaaaggatgtataaatttttacctgtttaaataaaatttaatttcaaatgt

SEQ ID NO: 24 - DNA, 1172 bp, Homo sapiens
gagacattcctcaattgcttagacatattctgagcctacagcagaggaacctccagtctcagcaccatgaatcaaactgcgattctgatttgctgccttatctttctgactctaagtggcattcaaggagtacctctctctagaaccgtacgctgtacctgcatcagcattagtaatcaacctgttaatccaaggtctttagaaaaacttgaaattattcctgcaagccaattttgtccacgtgttgagatcattgctacaatgaaaaagaagggtgagaagagatgtctgaatccagaatcgaaggccatcaagaatttactgaaagcagttagcaaggaaatgtctaaaagatctccttaaaaccagaggggagcaaaatcgatgcagtgcttccaaggatggaccacacagaggctgcctctcccatcacttccctacatggagtatatgtcaagccataattgttcttagtttgcagttacactaaaaggtgaccaatgatggtcaccaaatcagctgctactactcctgtaggaaggttaatgttcatcatcctaagctattcagtaataactctaccctggcactataatgtaagctctactgaggtgctatgttcttagtggatgttctgaccctgcttcaaatatttccctcacctttcccatcttccaagggtactaaggaatctttctgctttggggtttatcagaattctcagaatctcaaataactaaaaggtatgcaatcaaatctgctttttaaagaatgctctttacttcatggacttccactgccatcctcccaaggggcccaaattctttcagtggctacctacatacaattccaaacacatacaggaaggtagaaatatctgaaaatgtatgtgtaagtattcttatttaatgaaagactgtacaaagtataagtcttagatgtatatatttcctatattgttttcagtgtacatggaataacatgtaattaagtactatgtatcaatgagtaacaggaaaattttaaaaatacagatagatatatgctctgcatgttacataagataaatgtgctgaatggttttcaaataaaaatgaggtactctcctggaaatattaagaaagactatctaaatgttgaaagatcaaaaggttaataaagtaattataact

SEQ ID NO: 25 - DNA, 1216 bp, Homo sapiens
ttcggcacttgggagaagatgtttgaaaaaactgactctgctaatgagcctggactcagagctcaagtctgaactctacctccagacagaatgaagttcatctcgacatctctgcttctcatgctgctggtcagcagcctctctccagtccaaggtgttctggaggtctattacacaagcttgaggtgtagatgtgtccaagagagctcagtctttatccctagacgcttcattgatcgaattcaaatcttgccccgtgggaatggttgtccaagaaaagaaatcatagtctggaagaagaacaagtcaattgtgtgtgtggaccctcaagctgaatggatacaaagaatgatggaagtattgagaaaaagaagttcttcaactctaccagttccagtgtttaagagaaagattccctgatgctgatatttccactaagaacacctgcattcttcccttatccctgctctggattttagttttgtgcttagttaaatcttttccagggagaaagaacttccccatacaaataaggcatgaggactatgtgaaaaataaccttgcaggagctgatggggcaaactcaagcttcttcactcacagcaccctatatacacttggagtttgcattcttattcatcagggaggaaagtttctttgaaaatagttattcagttataagtaatacaggattattttgattatatacttgttgtttaatgtttaaaatttcttagaaaacaatggaatgagaatttaagcctcaaatttgaacatgtggcttgaattaagaagaaaattatggcatatattaaaagcaggcttctatgaaagactcaaaaagctgcctgggaggcagatggaacttgagcctgtcaagaggcaaaggaatccatgtagtagatatcctctgcttaaaaactcactacggaggagaattaagtcctacttttaaagaatttctttataaaatttactgtctaagattaatagcattcgaagatccccagacttcatagaatactcagggaaagcatttaaagggtgatgtacacatgtatcctttcacacatttgccttgacaaacttctttcactcacatctttttcactgactttttttgtgggggcggggccggggggactctggtatctaattctttaatgattcctataaatctaatgacattcaataaagttgagcaaacattttactt

SEQ ID NO: 26 - DNA, 5885 bp, Homo sapiens
ccacctctgtaaaagcggcccgggccggcccccggctccattttctcgcggcggccacacctggagccgcgcctttgggttgggctgggctgggccgcgcaaccgccacgggaagacagccctcggggcggggagggagagggtggccgggccggggggaggccggggccagggaggagccgagtgcgcgctcggggcaggcggcggcgcggagcggtgcggcggcgggaggcggaggcgagggtgcgatggcgcggagcccgggacgcgcgtacgccctgctgcttctcctgatctgctttaacgttggaagtggacttcacttacaggtcttaagcacaagaaatgaaaataagctgcttcctaaacatcctcatttagtgcggcaaaagcgcgcctggatcaccgcccccgtggctcttcgggagggagaggatctgtccaagaagaatccaattgccaagatacattctgatcttgcagaagaaagaggactcaaaattacttacaaatacactggaaaagggattacagagccaccttttggtatatttgtctttaacaaagatactggagaactgaatgttaccagcattcttgatcgagaagaaacaccattttttctgctaacaggttacgctttggatgcaagaggaaacaatgtagagaaacccttagagctacgcattaaggttcttgatatcaatgacaacgaaccagtgttcacacaggatgtctttgttgggtctgttgaagagttgagtgcagcacatactcttgtgatgaaaatcaatgcaacagatgcagatgagcccaataccctgaattcgaaaatttcctatagaatcgtatctctggagcctgcttatcctccagtgttctacctaaataaagatacaggagagatttatacaaccagtgttaccttggacagagaggaacacagcagctacactttgacagtagaagcaagagatggcaatggagaagttacagacaaacctgtaaaacaagctcaagttcagattcgtattttggatgtcaatgacaatatacctgtagtagaaaataaagtgcttgaagggatggttgaagaaaatcaagtcaacgtagaagttacgcgcataaaagtgttcgatgcagatgaaataggttctgataattggctggcaaattttacatttgcatcaggaaatgaaggaggttatttccacatagaaacagatgctcaaactaacgaaggaattgtgacccttattaaggaagtagattatgaagaaatgaagaatcttgacttcagtgttattgtcgctaataaagcagcttttcacaagtcgattaggagtaaatacaagcctacacccattcccatcaaggtcaaagtgaaaaatgtgaaagaaggcattcattttaaaagcagcgtcatctcaatttatgttagcgagagcatggatagatcaagcaaaggccaaataattggaaattttcaagcttttgatgaggacactggactaccagcccatgcaagatatgtaaaattagaagatagagataattggatctctgtggattctgtcacatctgaaattaaacttgcaaaacttcctgattttgaatctagatatgttcaaaatggcacatacactgtaaagattgtggccatatcagaagattatcctagaaaaaccatcactggcacagtccttatcaatgttgaagacatcaacgacaactgtcccacactgatagagcctgtgcagacaatctgtcacgatgcagagtatgtgaatgttactgcagaggacctggatggacacccaaacagtggccctttcagtttctccgtcattgacaaaccacctggcatggcagaaaaatggaaaatagcacgccaagaaagtaccagtgtgctgctgcaacaaagtgagaaaaagcttgggagaagtgaaattcagttcctgatttcagacaatcagggttttagttgtcctgaaaagcaggtccttacactcacagtttgtgagtgtctgcatggcagcggctgcagggaagcacagcatgactcctatgtgggcctgggacccgcagcaattgcgctcatgattttggcctttctgctcctgctattggtaccacttttactgctgatgtgccattgcggaaagggcgccaaaggctttacccccatacctggcaccatagagatgctgcatccttggaataatgaaggagcaccacctgaagacaaggtggtgccatcatttctgccagtggatcaagggggcagtctagtaggaagaaatggagtaggaggtatggccaaggaagccacgatgaaaggaagtagctctgcttccattgtcaaagggcaacatgagatgtccgagatggatggaaggtgggaagaacacagaagcctgctttctggtagagctacccagtttacaggggccacaggcgctatcatgaccactgaaaccacgaagaccgcaagggccacaggggcttccagagacatggccggagctcaggcagctgctgttgcactgaacgaagaattcttaagaaattatttcactgataaagcggcctcttacactgaggaagatgaaaatcacacagccaaagattgccttctggtttattctcaggaagaaactgaatcgctgaatgcttctattggttgttgcagttttattgaaggagagctagatgaccgcttcttagatgatttgggacttaaattcaagacactagctgaagtttgcctgggtcaaaaaatagatataaataaggaaattgagcagagacaaaaacctgccacagaaacaagtatgaacacagcttcacattcactctgtgagcaaactatggttaattcagagaatacctactcctctggcagtagcttcccagttccaaaatctttgcaagaagccaatgcagagaaagtaactcaggaaatagtcactgaaagatctgtgtcttctaggcaggcgcaaaaggtagctacacctcttcctgacccaatggcttctagaaatgtgatagcaacagaaacttcctatgtcacagggtccactatgccaccaaccactgtgatcctgggtcctagccagccacagagccttattgtgacagagagggtgtatgctccagcttctaccttggtagatcagccttatgctaatgaaggtacagttgtggtcactgaaagagtaatacagcctcatgggggtggatcgaatcctctggaaggcactcagcatcttcaagatgtaccttacgtcatggtgagggaaagagagagcttccttgcccccagctcaggtgtgcagcctactctggccatgcctaatatagcagtaggacagaatgtgacagtgacagaaagagttctagcacctgcttccactctgcaatccagttaccagattcccactgaaaattctatgacggctaggaacaccacggtgtctggagctggagtccctggccctctgccagattttggtttagaggaatctggtcattctaattctaccataaccacatcttccaccagagttaccaagcatagcactgtacagcattcttactcctaaacagcagtcagccacaaactgacccagagtttaattagcagtgactaatttcatgtttccaatgtacctgatttttcatgagccttacagacacacagagacacatacacattgatcttaaaatttttctcagtcactgatatgcaaaggaccacactgtctctgcttccaggagtattttagaaatgttccacaatttactgaagacatagagatgatgctgctgcttaggtgccttttagcaagctatgcaaacaatcctgataaaacaagatacatagagagtcaatctggcttctgagaatttaccaagtgaacagagtacctagttcatcagccgtccagtaaagcaacccaggaaactgactgggtctctttgcctaccgtattaacattaaacattgatgttctgtattctgtactttactgcacccagcagactttcaacaactcattgatccaaagatacatgcacagtctgagcaccagctatggtgctcataacttctttaagacttgaaccctttcaatctgtgtgattcattaaattggaccattgatgataagaatacacattgtatgtttctgtgcacatgacagtgtgtgtgtgtgcacgtacatactgtatagtcttaaaaatagcattatactggccaggggtggtggctaacgcctgtaatcccagcactttgggaggccgaggcgggtggatcaactgtggtcaggagtttgagatcagccaggccaacctggtgaaaccccgtctctactaaaaatacaaaaattagctgggcgtgatggtgggcgcctgtaatcccagctacttgggaggctgaggcaggagaatcacttgaacccgggaggcggaggttgcagtgagccgagatcgcaccattgcactccagtctgggcaacagagtgagattccgtctcaaaaaaaaaaagaaaaggaaaaaaaaatagcattatacctcttccttgtctcaaccgccatgaaaattctgaacactccaaattcagttgaataatccaaaacaaaatttataagtataaaataattttacttcttatagtaatagtatactttaaaaagcctcagggtatattatcttctaaacagctacaattcagtgcagctacattaaccaactatgttctctagttgagaacaactaggcctatttcactgctgtgtagcctcagtgcctaacatgggtgccaaataaatattcgtagaattacactgaattgtaaaaaccattcgtttttgtttacaattgccaaaaatctcaaaaggccctgtatttatgtaattctttgaaattattattttattttgatttctcagttattgactggctgggtgtgacttagtacataagtactcaatattataaaaacctcaaataattgacttgattttacacaacatccttcccttttctacaagttaatttttttacaaatcatttgggttatctcctaaataggttatattttattgcttctagaaacaatgtttcaaaatatatgtgcattatcagtaataatttgtataaatatttcccacaacaattttcataattttcaaagactaatttcttgactgaagatattttgctagggaagtgaaactttaaaattttgtagattttaaaaaatattgttgaatggtgtcatgcaaaggatttatatagtgtgctcccactaactgtacagatcaggacacatatttttagacatctaagtctgtagcttaaatggaggttactcttccatcatctagaattgtttacttagtaattgttgtttcttttattattatagacttactatcagttttattttgccaagtatgcaacaggtatatcactagtatatgaaaatgtaaatatcacttgtgtactcaaacaaaagttggtcttaagcttccaccttgagcagccttggaaacctaacctgcctcttttagcataatcacattttctaaatgattttctttgttcctgaaaaagtgatttgtattagttttacatttgttttttggaagattatatttgtatatgtatcatcataaaatatttaaataaaaagtatctttagagtgaccctttccccatagatttttatttctctattatattttacaaggaatataactcagtttgttagggagagtgccttaaaggcaggtgtttcttggactttgttatttaattagatctgcttgcaataaaaaaagttgtcggttatctaaaattcaaaaaaaaaaaaaaaaaa

SEQ ID NO: 27 - DNA, 1674 bp, Homo sapiens
ggggcggggcgtgccggggcggggcgtagtacggactgggcctggcctggggcgtccccgcgaagcctgggcctgtcaggcggttccgtccgggtctcggccaccgtcgagttccgtcgagttccgtcccggccctgctcacagcagcgccctcggagcgcccagcacctgcggccggccaggcagcgcgatcctgcggcgtctggccatcccgaatgctatggccgccgtcgccgtcttgcgggccttcggggcaagtgggcccatgtgtctccggcgcggcccctgggcccagctccccgcccgcttctgcagccgggacccggccggggcggggcggcgggagtcggagccgcggcccaccagcgcgcggcagctggacggcataaggaacatcgtcttgagcaatcccaagaagaggaacacgttgtcacttgcaatgctgaaatctctccaaagtgacattcttcatgacgctgacagcaacgatctgaaagtcattatcatctcggctgaggggcctgtgttttcttctgggcatgacttaaaggagctgacagaggagcaaggccgtgattaccatgccgaagtatttcagacctgttccaaggtcatgatgcacatccggaaccaccccgtccccgtcattgccatggtcaatggcctggccacggctgccggctgtcaactggttgccagctgcgacattgccgtggcgagcgacaagtcctcttttgccactcctggggtgaacgtcgggctcttctgttctacccctggggttgccttggcaagagcagtgcctagaaaggtggccttggagatgctctttactggtgagcccatttctgcccaggaggccctgctccacgggctgcttagcaaggtggtgccagaggcggagctgcaggaggagaccatgcggatcgctaggaagatcgcatcgctgagccgtccggtggtgtccctgggcaaagccaccttctacaagcagctgccccaggacctggggacggcttactacctcacctcccaggccatggtggacaacctggccctgcgggacgggcaggagggcatcacggccttcctccagaagagaaaacctgtctggtcacacgagccagtgtgagtggaggcagaggagtgaggcccacgggcagcgcccaggagcccaccttcccctctggcccagccaccactgcctctcagcttcaacaggtgacaggctgctttcgtgacttgatattggtgtcatagcatttggcctacattaaaagccacaatttcatggggaaaggacaaaatggagagtgactgaggtgctgacctcagtgcaaggctggtgaaccctgcagcgggccagctatggtgggaagcctggcatttggggtgctccttgcaacgtcttaagcaagcgacccccctgacatagcaaaaggtggcaacccatggaggcagaaagaaggacgccagcctgacccttatctgaaacgtcctaagcagagttaatcctggctgctcaggagaggcgacacatttcaaatctccacgagatattctccacacagaaaatcttcttgattctatagagacttaatcatgcctatggctttgaataatcttatgtgatttaaataaattaaatctttataaaaaaaaaaaaaaaaaaaa

SEQ ID NO: 28 - DNA, 3370 bp, Homo sapiens
gcccccgcccggcccgccccgctctcctagtcccttgcaacctggcgctgcatccgggccactgtcccaggtcccaggtcccggcccggagctatggagcggcgctggcccctggggctagggctggtgctgctgctctgcgccccgctgcccccgggggcgcgcgccaaggaagttactctgatggacacaagcaaggcacagggagagctgggctggctgctggatcccccaaaagatgggtggagtgaacagcaacagatactgaatgggacacccctctacatgtaccaggactgcccaatgcaaggacgcagagacactgaccactggcttcgctccaattggatctaccgcggggaggaggcttcccgcgtccacgtggagctgcagttcaccgtgcgggactgcaagagtttccctgggggagccgggcctctgggctgcaaggagaccttcaaccttctgtacatggagagtgaccaggatgtgggcattcagctccgacggcccttgttccagaaggtaaccacggtggctgcagaccagagcttcaccattcgagaccttgcgtctggctccgtgaagctgaatgtggagcgctgctctctgggccgcctgacccgccgtggcctctacctcgctttccacaacccgggtgcctgtgtggccctggtgtctgtccgggtcttctaccagcgctgtcctgagaccctgaatggcttggcccaattcccagacactctgcctggccccgctgggttggtggaagtggcgggcacctgcttgccccacgcgcgggccagccccaggccctcaggtgcaccccgcatgcactgcagccctgatggcgagtggctggtgcctgtaggacggtgccactgtgagcctggctatgaggaaggtggcagtggcgaagcatgtgttgcctgccctagcggctcctaccggatggacatggacacaccccattgtctcacgtgcccccagcagagcactgctgagtctgagggggccaccatctgtacctgtgagagcggccattacagagctcccggggagggcccccaggtggcatgcacaggtcccccctcggccccccgaaacctgagcttctctgcctcagggactcagctctccctgcgttgggaacccccagcagatacggggggacgccaggatgtcagatacagtgtgaggtgttcccagtgtcagggcacagcacaggacggggggccctgccagccctgtggggtgggcgtgcacttctcgccgggggcccgggcgctcaccacacctgcagtgcatgtcaatggccttgaaccttatgccaactacacctttaatgtggaagcccaaaatggagtgtcagggctgggcagctctggccatgccagcacctcagtcagcatcagcatggggcatgcagagtcactgtcaggcctgtctctgagactggtgaagaaagaaccgaggcaactagagctgacctgggcggggtcccggccccgaagccctggggcgaacctgacctatgagctgcacgtgctgaaccaggatgaagaacggtaccagatggttctagaacccagggtcttgctgacagagctgcagcctgacaccacatacatcgtcagagtccgaatgctgaccccactgggtcctggccctttctcccctgatcatgagtttcggaccagcccaccagtgtccaggggcctgactggaggagagattgtagccgtcatctttgggctgctgcttggtgcagccttgctgcttgggattctcgttttccggtccaggagagcccagcggcagaggcagcagaggcacgtgaccgcgccaccgatgtggatcgagaggacaagctgtgctgaagccttatgtggtacctccaggcatacgaggaccctgcacagggagccttggactttacccggaggctggtctaattttccttcccgggagcttgatccagcgtggctgatggtggacactgtcataggagaaggagagtttggggaagtgtatcgagggaccctcaggctccccagccaggactgcaagactgtggccattaagaccttaaaagacacatccccaggtggccagtggtggaacttccttcgagaggcaactatcatgggccagtttagccacccgcatattctgcatctggaaggcgtcgtcacaaagcgaaagccgatcatgatcatcacagaatttatggagaatgcagccctggatgccttcctgagggagcgggaggaccagctggtccctgggcagctagtggccatgctgcagggcatagcatctggcatgaactacctcagtaatcacaattatgtccaccgggacctggctgccagaaacatcttggtgaatcaaaacctgtgctgcaaggtgtctgactttggcctgactcgcctcctggatgactttgatggcacatacgaaacccagggaggaaagatccctatccgttggacagcccctgaagccattgcccatcggatcttcaccacagccagcgatgtgtggagctttgggattgtgatgtgggaggtgctgagctttggggacaagccttatggggagatgagcaatcaggaggttatgaagagcattgaggatgggtaccggttgccccctcctgtggactgccctgcccctctgtatgagctcatgaagaactgctgggcatatgaccgtgcccgccggccacacttccagaagcttcaggcacatctggagcaactgcttgccaacccccactccctgcggaccattgccaactttgaccccagggtgactcttcgcctgcccagcctgagtggctcagatgggatcccgtatcgaaccgtctctgagtggctcgagtccatacgcatgaaacgctacatcctgcacttccactcggctgggctggacaccatggagtgtgtgctggagctgaccgctgaggacctgacgcagatgggaatcacactgcccgggcaccagaagcgcattctttgcagtattcagggattcaaggactgatccctcctctcaccccatgcccaatcagggtgcaaggagcaaggacggggccaaggtcgctcatggtcactccctgcgccccttcccacaacctgccagactaggctatcggtgctgcttctgcccgctttaaggagaaccctgctctgcaccccagaaaacctctttgttttaaaagggaggtgggggtagaagtaaaaggatgatcatgggagggagctcaggggttaatatatatacatacatacacatatatatattgttgtaaataaacaggaaatgattttctgcctccatcccacccatcagggctgcaggcact

SEQ ID NO: 29 - DNA, 662 bp, Homo sapiens
accgccgacgcagacccctctctgcacgccagcccgcccgcacccaccatggccacagttcagcagctggaaggaagatggcgcctggtggacagcaaaggctttgatgaatacatgaaggagctaggagtgggaatagctttgcgaaaaatgggcgcaatggccaagccagattgtatcatcacttgtgatggtaaaaacctcaccataaaaactgagagcactttgaaaacaacacagttttcttgtaccctgggagagaagtttgaagaaaccacagctgatggcagaaaaactcagactgtctgcaactttacagatggtgcattggttcagcatcaggagtgggatgggaaggaaagcacaataacaagaaaattgaaagatgggaaattagtggtggagtgtgtcatgaacaatgtcacctgtactcggatctatgaaaaagtagaataaaaattccatcatcactttggacaggagttaattaagagaatgaccaagctcagttcaatgagcaaatctccatactgtttctttcttttttttttcattactgtgttcaattatctttatcataaacattttacatgcagctatttcaaagtgtgttggattaattaggatcatccctttggttaataaataaatgtgtttgtgct

SEQ ID NO: 30 - DNA, 3270 bp, Homo sapiens
  misc_feature (2708)..(2708): n is a, c, g, or t
  misc_feature (2746)..(2746): n is a, c, g, or t
  misc_feature (2806)..(2806): n is a, c, g, or t
caaaaagtgtgtggaaaggtggattgagggagcgggacccccgcgggacccgagggggcggcaggcggggaacggggagtcagcccgcgctgtgtctcggggccggccggcaggaaggagccatggctctggacgggataaggatgccagatggctgctacgcggacgggacgtgggaactgagtgtccatgtgacggacctgaaccgcgatatcaccctgagagtgaccggcgaggtgcacattggaggcgtgatgcttaagctggtggagaaactcgatgtaaaaaaagattggtctgaccatgctctctggtgggaaaagaagagaacttggcttctgaagacacattggaccttagataagtatggtattcaggcagatgctaagcttcagttcacccctcagcacaaactgctccgcctgcagcttcccaacatgaagtatgtgaaggtgaaagtgaatttctctgatagagtcttcaaagctgtttctgacatctgtaagacttttaatatcagacaccccgaagaactttctctcttaaagaaacccagagatccaacaaagaaaaaaaagaagaagctagatgaccagtctgaagatgaggcacttgaattagaggggcctcttatcactcctggatcaggaagtatatattcaagcccaggactgtatagtaaaacaatgacccccacttatgatgctcatgatggaagccccttgtcaccaacttctgcttggtttggtgacagtgctttgtcagaaggcaatcctggtatacttgctgtcagtcaaccaatcacgtcaccagaaatcttggcaaaaatgttcaagcctcaagctcttcttgataaagcaaaaatcaaccaaggatggcttgattcctcaagatctctcatggaacaagatgtgaaggaaaatgaggccttgctgctccgattcaagtattacagcttttttgatttgaatccaaagtatgatgcaatcagaatcaatcagctttatgagcaggccaaatgggccattctcctggaagagattgaatgcacagaagaagaaatgatgatgtttgcagccctgcagtatcatatcaataagctgtcaatcatgacatcagagaatcatttgaacaacagtgacaaagaagttgatgaagttgatgctgccctttcagacctggagattactctggaagggggtaaaacgtcaacaattttgggtgacattacttccattcctgaacttgctgactacattaaagttttcaagccaaaaaagctgactctgaaaggttacaaacaatattggtgcaccttcaaagacacatccatttcttgttataagagcaaagaagaatccagtggcacaccagctcatcagatgaacctcaggggatgtgaagttaccccagatgtaaacatttcaggccaaaaatttaacattaaactcctgattccagttgcagaaggcatgaatgaaatctggcttcgttgtgacaatgaaaaacagtatgcacactggatggcagcctgcagattagcctccaaaggcaagaccatggcggacagttcttacaacttagaagttcagaatattctttcctttctgaagatgcagcatttaaacccagatcctcagttaataccagagcagatcacgactgatataactcctgaatgtttggtgtctccccgctatctaaaaaagtataagaacaagcagataacagcgagaatcttggaggcccatcagaatgtagctcagatgagtctaattgaagccaagatgagatttattcaagcttggcagtcactacctgaatttggcatcactcacttcattgcaaggttccaagggggcaaaaaagaagaacttattggaattgcatacaacagactgattcggatggatgccagcactggagatgcaattaaaacatggcgtttcagcaacatgaaacagtggaatgtcaactgggaaatcaaaatggtcaccgtagagtttgcagatgaagtacgattgtccttcatttgtactgaagtagattgcaaagtggttcatgaattcattggtggctacatatttctctcaacacgtgcaaaagaccaaaacgagagtttagatgaagagatgttctacaaacttaccagtggttgggtgtgaatagaaatactgtttaatgaaactccacggccataacaatatttaactttaaaagctgtttgttatatgctgcttaataaagtaagcttgaaatttatcattttatcatgaaaacttctttgccttaccagaccagttaatatgtgcactaaacaagcacgactattaatctatcatgttatgatataataaacttgaatttggcacacattccttagggccatgaattgaaaactgaaatagtgggcaaatcaggaacaaaccatcactgatttactgatttaagctagccaaactgtaagaaacaagccatctattttaaagctatccagggcttaacctatatgaactctatttatcatgtctaatgcatgtgatttaatgtatgtttaatttgatatcatgttttaaaatatcctacttctggtagccatttaattcctccccctacccccaaataaatcaggcatgcaggaggcctgatatttagtaatgtcattgtgtttgaccttgaaggaaaatgctattagtccgtcgtgcttnatttgtttttgtccttgaataagcatgttatgtatatngtctcgtgtttttatttttacaccatattgtattacacttttagtattcaccagcataancactgtctgcctaaaatatgcaactctttgcattacaatatgaagtaaagttctatgaagtatgcattttgtgtaactaatgtaaaaacacaaattttataaaattgtacagttttttaaaaactactcacaactagcagatggcttaaatgtagcaatctctgcgttaattaaatgcctttaagagatataattaacgtgcagttttaatatctactaaattaagaatgacttcattatgatcatgatttgccacaatgtccttaactctaatgcctggactggccatgttctagtctgttgcgctgttacaatctgtattggtgctagtcagaaaattcctagctcacatagcccaaaagggtgcgagggagaggtggattaccagtattgttcaataatccatggttcaaagactgtataaatgcattttattttaaataaaagcaaaacttttatttaaa

SEQ ID NO: 31 - DNA, 801 bp, Homo sapiens
caccgcaccctcggactgccccaaggcccccgccgccgctccagcgccgcgcagccaccgccgccgccgccgcctctccttagtcgccgccatgacgaccgcgtccacctcgcaggtgcgccagaactaccaccaggactcagaggccgccatcaaccgccagatcaacctggagctctacgcctcctacgtttacctgtccatgtcttactactttgaccgcgatgatgtggctttgaagaactttgccaaatactttcttcaccaatctcatgaggagagggaacatgctgagaaactgatgaagctgcagaaccaacgaggtggccgaatcttccttcaggatatcaagaaaccagactgtgatgactgggagagcgggctgaatgcaatggagtgtgcattacatttggaaaaaaatgtgaatcagtcactactggaactgcacaaactggccactgacaaaaatgacccccatttgtgtgacttcattgagacacattacctgaatgagcaggtgaaagccatcaaagaattgggtgaccacgtgaccaacttgcgcaagatgggagcgcccgaatctggcttggcggaatatctctttgacaagcacacctgggagacagtgataatgaaagctaagcctcgggctaatttcccatagccgtggggtgacttcctggtcaccaaggcagtgcatgcatgttggggtttcctttaccttttctataagttgtaccaaaacatccacttaagttctttgatttgtaccattccttcaaataaagaaatttggtaccc

SEQ ID NO: 32 - DNA, 1614 bp, Homo sapiens
gcgggcgtgctgccgccgctgccgccgccgccgcagcccggccgcgccccgccgccgccgccgccgccatgggctgcctcgggaacagtaagaccgaggaccagcgcaacgaggagaaggcgcagcgtgaggccaacaaaaagatcgagaagcagctgcagaaggacaagcaggtctaccgggccacgcaccgcctgctgctgctgggtgctggagaatctggtaaaagcaccattgtgaagcagatgaggatcctgcatgttaatgggtttaatggagagggcggcgaagaggacccgcaggctgcaaggagcaacagcgatggtgagaaggcaaccaaagtgcaggacatcaaaaacaacctgaaagaggcgattgaaaccattgtggccgccatgagcaacctggtgccccccgtggagctggccaaccccgagaaccagttcagagtggactacatcctgagtgtgatgaacgtgcctgactttgacttccctcccgaattctatgagcatgccaaggctctgtgggaggatgaaggagtgcgtgcctgctacgaacgctccaacgagtaccagctgattgactgtgcccagtacttcctggacaagatcgacgtgatcaagcaggctgactatgtgccgagcgatcaggacctgcttcgctgccgtgtcctgacttctggaatctttgagaccaagttccaggtggacaaagtcaacttccacatgtttgacgtgggtggccagcgcgatgaacgccgcaagtggatccagtgcttcaacgatgtgactgccatcatcttcgtggtggccagcagcagctacaacatggtcatccgggaggacaaccagaccaaccgcctgcaggaggctctgaacctcttcaagagcatctggaacaacagatggctgcgcaccatctctgtgatcctgttcctcaacaagcaagatctgctcgctgagaaagtccttgctgggaaatcgaagattgaggactactttccagaatttgctcgctacactactcctgaggatgctactcccgagcccggagaggacccacgcgtgacccgggccaagtacttcattcgagatgagtttctgaggatcagcactgccagtggagatgggcgtcactactgctaccctcatttcacctgcgctgtggacactgagaacatccgccgtgtgttcaacgactgccgtgacatcattcagcgcatgcaccttcgtcagtacgagctgctctaagaagggaacccccaaatttaattaaagccttaagcacaattaattaaaagtgaaacgtaattgtacaagcagttaatcacccaccatagggcatgattaacaaagcaacctttcccttcccccgagtgattttgcgaaacccccttttcccttcagcttgcttagatgttccaaatttagaaagcttaaggcggcctacagaaaaaggaaaaaaggccacaaaagttccctctcactttcagtaaaaataaataaaacagcagcagcaaacaaataaaatgaaataaaagaaacaaatgaaataaatattgtgttgtgcagcattaaaaaaaatcaaaataaaaattaaatgtgagcaaag

SEQ ID NO: 33 - DNA, 2152 bp, Homo sapiens
gctgctgcccaaggaccgcggagtcggacgcaggcagaccatgtggaccctggtgagctgggtggccttaacagcagggctggtggctggaacgcggtgcccagatggtcagttctgccctgtggcctgctgcctggaccccggaggagccagctacagctgctgccgtccccttctggacaaatggcccacaacactgagcaggcatctgggtggcccctgccaggttgatgcccactgctctgccggccactcctgcatctttaccgtctcagggacttccagttgctgccccttcccagaggccgtggcatgcggggatggccatcactgctgcccacggggcttccactgcagtgcagacgggcgatcctgcttccaaagatcaggtaacaactccgtgggtgccatccagtgccctgatagtcagttcgaatgcccggacttctccacgtgctgtgttatggtcgatggctcctgggggtgctgccccatgccccaggcttcctgctgtgaagacagggtgcactgctgtccgcacggtgccttctgcgacctggttcacacccgctgcatcacacccacgggcacccaccccctggcaaagaagctccctgcccagaggactaacagggcagtggccttgtccagctcggtcatgtgtccggacgcacggtcccggtgccctgatggttctacctgctgtgagctgcccagtgggaagtatggctgctgcccaatgcccaacgccacctgctgctccgatcacctgcactgctgcccccaagacactgtgtgtgacctgatccagagtaagtgcctctccaaggagaacgctaccacggacctcctcactaagctgcctgcgcacacagtgggggatgtgaaatgtgacatggaggtgagctgcccagatggctatacctgctgccgtctacagtcgggggcctggggctgctgcccttttacccaggctgtgtgctgtgaggaccacatacactgctgtcccgcggggtttacgtgtgacacgcagaagggtacctgtgaacaggggccccaccaggtgccctggatggagaaggccccagctcacctcagcctgccagacccacaagccttgaagagagatgtcccctgtgataatgtcagcagctgtccctcctccgatacctgctgccaactcacgtctggggagtggggctgctgtccaatcccagaggctgtctgctgctcggaccaccagcactgctgcccccagggctacacgtgtgtagctgaggggcagtgtcagcgaggaagcgagatcgtggctggactggagaagatgcctgcccgccgggcttccttatcccaccccagagacatcggctgtgaccagcacaccagctgcccggtggggcagacctgctgcccgagcctgggtgggagctgggcctgctgccagttgccccatgctgtgtgctgcgaggatcgccagcactgctgcccggctggctacacctgcaacgtgaaggctcgatcctgcgagaaggaagtggtctctgcccagcctgccaccttcctggcccgtagccctcacgtgggtgtgaaggacgtggagtgtggggaaggacacttctgccatgataaccagacctgctgccgagacaaccgacagggctgggcctgctgtccctaccgccagggcgtctgttgtgctgatcggcgccactgctgtcctgctggcttccgctgcgcagccaggggtaccaagtgtttgcgcagggaggccccgcgctgggacgcccctttgagggacccagccttgagacagctgctgtgagggacagtactgaagactctgcagccctcgggaccccactcggagggtgccctctgctcaggcctccctagcacctccccctaaccaaattctccctggaccccattctgagctccccatcaccatgggaggtggggcctcaatctaaggccttccctgtcagaagggggttgtggcaaaagccacattacaagctgccatcccctccccgtttcagtggaccctgtggccaggtgcttttccctatccacaggggtgtttgtgtgtgtgcgcgtgtgcgtttcaataaagtttgtacactttcaaaaaaaaaaaaaaaaaaaaaaaaaa

SEQ ID NO: 34 - DNA, 2381 bp, Homo sapiens
attcggccgaaggagctacgcgggccacgctgctggctggcctgacctaggcgcgcggggtcgggcggccgcgcgggcgggctgagtgagcaagacaagacactcaagaagagcgagctgcgcctgggtcccggccaggcttgcacgcagaggcgggcggcagacggtgcccggcggaatctcctgagctccgccgcccagctctggtgccagcgcccagtggccgccgcttcgaaagtgactggtgcctcgccgcctcctctcggtgcgggaccatgaagctgctgccgtcggtggtgctgaagctctttctggctgcagttctctcggcactggtgactggcgagagcctggagcggcttcggagagggctagctgctggaaccagcaacccggaccctcccactgtatccacggaccagctgctacccctaggaggcggccgggaccggaaagtccgtgacttgcaagaggcagatctggaccttttgagagtcactttatcctccaagccacaagcactggccacaccaaacaaggaggagcacgggaaaagaaagaagaaaggcaaggggctagggaagaagagggacccatgtcttcggaaatacaaggacttctgcatccatggagaatgcaaatatgtgaaggagctccgggctccctcctgcatctgccacccgggttaccatggagagaggtgtcatgggctgagcctcccagtggaaaatcgcttatatacctatgaccacacaaccatcctggccgtggtggctgtggtgctgtcatctgtctgtctgctggtcatcgtggggcttctcatgtttaggtaccataggagaggaggttatgatgtggaaaatgaagagaaagtgaagttgggcatgactaattcccactgagagagacttgtgctcaaggaatcggctggggactgctacctctgagaagacacaaggtgatttcagactgcagaggggaaagacttccatctagtcacaaagactccttcgtccccagttgccgtctaggattgggcctcccataattgctttgccaaaataccagagccttcaagtgccaaacagagtatgtccgatggtatctgggtaagaagaaagcaaaagcaagggaccttcatgcccttctgattcccctccaccaaaccccacttcccctcataagtttgtttaaacacttatcttctggattagaatgccggttaaattccatatgctccaggatctttgactgaaaaaaaaaaagaagaagaagaaggagagcaagaaggaaagatttgtgaactggaagaaagcaacaaagattgagaagccatgtactcaagtaccaccaagggatctgccattgggaccctccagtgctggatttgatgagttaactgtgaaataccacaagcctgagaactgaattttgggacttctacccagatggaaaaataacaactatttttgttgttgttgtttgtaaatgcctcttaaattatatatttattttattctatgtatgttaatttatttagtttttaacaatctaacaataatatttcaagtgcctagactgttactttggcaatttcctggccctccactcctcatccccacaatctggcttagtgccacccacctttgccacaaagctaggatggttctgtgacccatctgtagtaatttattgtctgtctacatttctgcagatcttccgtggtcagagtgccactgcgggagctctgtatggtcaggatgtaggggttaacttggtcagagccactctatgagttggacttcagtcttgcctaggcgattttgtctaccatttgtgttttgaaagcccaaggtgctgatgtcaaagtgtaacagatatcagtgtctccccgtgtcctctccctgccaagtctcagaagaggttgggcttccatgcctgtagctttcctggtccctcacccccatggccccaggcccacagcgtgggaactcactttcccttgtgtcaagacatttctctaactcctgccattcttctggtgctactccatgcaggggtcagtgcagcagaggacagtctggagaaggtattagcaaagcaaaaggctgagaaggaacagggaacattggagctgactgttcttggtaactgattacctgccaattgctaccgagaaggttggaggtggggaaggctttgtataatcccacccacctcaccaaaacgatgaagttatgctgtcatggtcctttctggaagtttctggtgccatttctgaactgttacaacttgtatttccaaacctggttcatatttatactttgcaatccaaataaagataacccttattccataaaaaaaaaaaaaaaaaaaaaaaa

SEQ ID NO: 35 - DNA, 1115 bp, Homo sapiens
ctgctctggcccctggtcctgtcctgttctccagcatggtgtgtctgaggctccctggaggctcctgcatggcagttctgacagtgacactgatggtgctgagctccccactggctttggctggggacaccagaccacgtttcttggagtactctacgtctgagtgtcatttcttcaatgggacggagcgggtgcggtacctggacagatacttccataaccaggaggagaacgtgcgcttcgacagcgacgtgggggagttccgggcggtgacggagctggggcggcctgatgccgagtactggaacagccagaaggacctcctggagcagaagcggggccgggtggacaactactgcagacacaactacggggttgtggagagcttcacagtgcagcggcgagtccatcctaaggtgactgtgtatccttcaaagacccagcccctgcagcaccataacctcctggtctgttctgtgagtggtttctatccaggcagcattgaagtcaggtggttccggaatggccaggaagagaagactggggtggtgtccacaggcctgatccacaatggagactggaccttccagaccctggtgatgctggaaacagttcctcggagtggagaggtttacacctgccaagtggagcacccaagcgtgacaagccctctcacagtggaatggagagcacggtctgaatctgcacagagcaagatgctgagtggagtcgggggctttgtgctgggcctgctcttccttggggccgggctgttcatctacttcaggaatcagaaaggacactctggacttcagccaagaggattcctgagctgaagtgcagatgacacattcaaagaagaactttctgccccagctttgcaggatgaaaagctttccctcctggctgttattcttccacaagagagggctttctcaggacctggttgctactggttcagcaactgcagaaaatgtcctcccttgtggcttcctcagctcctgttcttggcctgaagccccacagctttgatggcagtgcctcatcttcaacttttgtgctcccctttgcctaaaccctatggcctcctgtgcatctgtactcaccctgtacca

SEQ ID NO: 36 - DNA, 1172 bp, Homo sapiens
ccagcatggtgtgtctgaagctccctggaggttcctacatggcaaagctgacagtgacactgatggtgctgagctccccactggctttggctggggacacccgaccacgtttcttgcagcaggataagtatgagtgtcatttcttcaacgggacggagcgggtgcggttcctgcacagagacatctataaccaagaggaggacttgcgcttcgacagcgacgtgggggagtaccgggcggtgacggagctggggcggcctgacgctgagtactggaacagccagaaggacttcctggaagacaggcgcgccgcggtggacacctactgcagacacaactacggggttggtgagagcttcacagtgcagcggcgagttgagcctaaggtgactgtgtatcctgcaaggacccagaccctgcagcaccacaacctcctggtctgctctgtgaatggtttctatccaggcagcattgaagtcaggtggttccggaacagccaggaagagaaggctggggtggtgtccacaggcctgattcagaatggagactggaccttccagaccctggtgatgctggaaacagttcctcgaagtggagaggtttacacctgccaagtggagcacccaagcgtgacgagccctctcacagtggaatggagagcacagtctgaatctgcacagagcaagatgctgagtggagtcgggggctttgtgctgggcctgctcttccttggggccgggctattcatctacttcaagaatcagaaagggcactctggacttcacccaacaggactcgtgagctgaagtgcagatgaccacattcaagggggaaccttctgccccagctttgcatgatgaaaagctttcctgcttggctcttattcttccacaagagaggactttctcaggccctggttgctaccggttcagcaactctgcagaaaatgtccatccttgtggcttcctcagctcctgccccttggcctgaagtcccagcattgatggcagtgcctcatcttcaactttagtgctcccctttacctaaccctacggcctcccatgcatctgtactccccctgtgtgccacaaatgcactacgttattaaatttttctgaagcccagagttaaaaatcatctgtccacctggctccaaagacaaaaaataaaaa

SEQ ID NO: 37 - DNA, 1642 bp, Homo sapiens
ccagatctcagaggagcctggctaagcaaaaccctgcagaacggctgcctaatttacagcaaccatgagtacaaatggtgatgatcatcaggtcaaggatagtctggagcaattgagatgtcactttacatgggagttatccattgatgacgatgaaatgcctgatttagaaaacagagtcttggatcagattgaattcctagacaccaaatacagtgtgggaatacacaacctactagcctatgtgaaacacctgaaaggccagaatgaggaagccctgaagagcttaaaagaagctgaaaacttaatgcaggaagaacatgacaaccaagcaaatgtgaggagtctggtgacctggggcaactttgcctggatgtattaccacatgggcagactggcagaagcccagacttacctggacaaggtggagaacatttgcaagaagctttcaaatcccttccgctatagaatggagtgtccagaaatagactgtgaggaaggatgggccttgctgaagtgtggaggaaagaattatgaacgggccaaggcctgctttgaaaaggtgcttgaagtggaccctgaaaaccctgaatccagcgctgggtatgcgatctctgcctatcgcctggatggctttaaattagccacaaaaaatcacaagccattttctttgcttcccctaaggcaggctgtccgcttaaatccagacaatggatatattaaggttctccttgccctgaagcttcaggatgaaggacaggaagctgaaggagaaaagtacattgaagaagctctagccaacatgtcctcacagacctatgtctttcgatatgcagccaagttttaccgaagaaaaggctctgtggataaagctcttgagttattaaaaaaggccttgcaggaaacacccacttctgtcttactgcatcaccagatagggctttgctacaaggcacaaatgatccaaatcaaggaggctacaaaagggcagcctagagggcagaacagagaaaagctagacaaaatgataagatcagccatatttcattttgaatctgcagtggaaaaaaagcccacatttgaggtggctcatctagacctggcaagaatgtatatagaagcaggcaatcacagaaaagctgaagagaattttcaaaaattgttatgcatgaaaccagtggtagaagaaacaatgcaagacatacatttctactatggtcggtttcaggaatttcaaaagaaatctgacgtcaatgcaattatccattatttaaaagctataaaaatagaacaggcatcattaacaagggataaaagtatcaattctttgaagaaattggttttaaggaaacttcggagaaaggcattagatctggaaagcttgagcctccttgggttcgtctacaaattggaaggaaatatgaatgaagccctggagtactatgagcgggccctgagactggctgctgactttgagaactctgtgagacaaggtccttaggcacccagatatcagccactttcacatttcatttcattttatgctaacatttactaatcatcttttctgcttactgttttcagaaacattataattcactgtaatgatgtaattcttgaataataaatctgacaaaatatt

SEQ ID NO: 38 - DNA, 1990 bp, Homo sapiens
gtggaaacctcttcagcatttgcttggaatcagtaagctaaaaacaaaatcaaccgggaccccagcttttcagaactgcagggaaacagccatcatgagtgaggtcaccaagaattccctggagaaaatcctcccacagctgaaatgccatttcacctggaacttattcaaggaagacagtgtctcaagggatctagaagatagagtgtgtaaccagattgaatttttaaacactgagttcaaagctacaatgtacaacttgttggcctacataaaacacctagatggtaacaacgaggcagccctggaatgcttacggcaagctgaagagttaatccagcaagaacatgctgaccaagcagaaatcagaagtctagtcacttggggaaactacgcctgggtctactatcacttgggcagactctcagatgctcagatttatgtagataaggtgaaacaaacctgcaagaaattttcaaatccatacagtattgagtattctgaacttgactgtgaggaagggtggacacaactgaagtgtggaagaaatgaaagggcgaaggtgtgttttgagaaggctctggaagaaaagcccaacaacccagaattctcctctggactggcaattgcgatgtaccatctggataatcacccagagaaacagttctctactgatgttttgaagcaggccattgagctgagtcctgataaccaatacgtcaaggttctcttgggcctgaaactgcagaagatgaataaagaagctgaaggagagcagtttgttgaagaagccttggaaaagtctccttgccaaacagatgtcctccgcagtgcagccaaattttacagaagaaaaggtgacctagacaaagctattgaactgtttcaacgggtgttggaatccacaccaaacaatggctacctctatcaccagattgggtgctgctacaaggcaaaagtaagacaaatgcagaatacaggagaatctgaagctagtggaaataaagagatgattgaagcactaaagcaatatgctatggactattcgaataaagctcttgagaagggactgaatcctctgaatgcatactccgatctcgctgagttcctggagacggaatgttatcagacaccattcaataaggaagtccctgatgctgaaaagcaacaatcccatcagcgctactgcaaccttcagaaatataatgggaagtctgaagacactgctgtgcaacatggtttagagggtttgtccataagcaaaaaatcaactgacaaggaagagatcaaagaccaaccacagaatgtatccgaaaatctgcttccacaaaatgcaccaaattattggtatcttcaaggattaattcataagcagaatggagatctgctgcaagcagccaaatgttatgagaaggaactgggccgcctgctaagggatgccccttcaggcataggcagtattttcctgtcagcatctgagcttgaggatggtagtgaggaaatgggccagggcgcagtcagctccagtcccagagagctcctctctaactcagagcaactgaactgagacagaggaggaaaacagagcatcagaagcctgcagtggtggttgtgacgggtaggaggataggaagacagggggccccaacctgggattgctgagcagggaagctttgcatgttgctctaaggtacatttttaaagagttgttttttggccgggcgcagtggctcatgcctgtaatcccagcactttgggaggccgaggtgggcggatcacgaggtctggagtttgagaccatcctggctaacacagtgaaatcccgtctctactaaaaatacaaaaaattagccaggcgtggtggctggcacctgtagtcccagctacttgggaggctgaggcaggagaatggcgtgaacctggaaggaagaggttgcagtgagccaagattgcgcccctgcactccagcctgggcaacagagcaagactc

SEQ ID NO: 39 - DNA, 678 bp, Homo sapiens
aggaaaaggaaactgttgagaaaccgaaactactggggaaagggagggctcactgagaaccatcccagtaacccgaccgccgctggtcttcgctggacaccatgaatcacactgtccaaaccttcttctctcctgtcaacagtggccagccccccaactatgagatgctcaaggaggagcacgaggtggctgtgctgggggcgccccacaaccctgctcccccgacgtccaccgtgatccacatccgcagcgagacctccgtgcccgaccatgtcgtctggtccctgttcaacaccctcttcatgaacccctgctgcctgggcttcatagcattcgcctactccgtgaagtctagggacaggaagatggttggcgacgtgaccggggcccaggcctatgcctccaccgccaagtgcctgaacatctgggccctgattctgggcatcctcatgaccattctgctcatcgtcatcccagtgctgatcttccaggcctatggatagatcaggaggcatcactgaggccaggagctctgcccatgacctgtatcccacgtactccaacttccattcctcgccctgcccccggagccgagtcctgtatcagccctttatcctcacacgcttttctacaatggcattcaataaagtgcacgtgtttctggtgctaaaaaaaaaa

SEQ ID NO: 40 - DNA, 2755 bp, Homo sapiens
ttaggacggggcgatggcggctgagaggagctgcgcgtgcgcgaacatgtaactggtgggatctgcggcggctcccagatgatggtcgtcctcctgggcgcgacgaccctagtgctcgtcgccgtgggcccatgggtgttgtccgcagccgcaggtggaaaaaatctaaaatctcctcaaaaagtagaggtcgacatcatagatgacaactttatcctgaggtggaacaggagcgatgagtctgtcgggaatgtgactttttcattcgattatcaaaaaactgggatggataattggataaaattgtctgggtgtcagaatattactagtaccaaatgcaacttttcttcactcaagctgaatgtttatgaagaaattaaattgcgtataagagcagaaaaagaaaacacttcttcatggtatgaggttgactcatttacaccatttcgcaaagctcagattggtcctccagaagtacatttagaagctgaagataaggcaatagtgatacacatctctcctggaacaaaagatagtgttatgtgggctttggatggtttaagctttacatatagcttacttatctggaaaaactcttcaggtgtagaagaaaggattgaaaatatttattccagacataaaatttataaactctcaccagagactacttattgtctaaaagttaaagcagcactacttacgtcatggaaaattggtgtctatagtccagtacattgtataaagaccacagttgaaaatgaactacctccaccagaaaatatagaagtcagtgtccaaaatcagaactatgttcttaaatgggattatacatatgcaaacatgacctttcaagttcagtggctccacgcctttttaaaaaggaatcctggaaaccatttgtataaatggaaacaaatacctgactgtgaaaatgtcaaaactacccagtgtgtctttcctcaaaacgttttccaaaaaggaatttaccttctccgcgtacaagcatctgatggaaataacacatctttttggtctgaagagataaagtttgatactgaaatacaagctttcctacttcctccagtctttaacattagatcccttagtgattcattccatatctatatcggtgctccaaaacagtctggaaacacgcctgtgatccaggattatccactgatttatgaaattattttttgggaaaacacttcaaatgctgagagaaaaattatcgagaaaaaaactgatgttacagttcctaatttgaaaccactgactgtatattgtgtgaaagccagagcacacaccatggatgaaaagctgaataaaagcagtgtttttagtgacgctgtatgtgagaaaacaaaaccaggaaatacctctaaaatttggcttatagttggaatttgtattgcattatttgctctcccgtttgtcatttatgctgcgaaagtcttcttgagatgcatcaattatgtcttctttccatcacttaaaccttcttccagtatagatgagtatttctctgaacagccattgaagaatcttctgctttcaacttctgaggaacaaatcgaaaaatgtttcataattgaaaatataagcacaattgctacagtagaagaaactaatcaaactgatgaagatcataaaaaatacagttcccaaactagccaagattcaggaaattattctaatgaagatgaaagcgaaagtaaaacaagtgaagaactacagcaggactttgtatgaccagaaatgaactgtgtcaagtataaggtttttcagcaggagttacactgggagcctgaggtcctcaccttcctctcagtaactacagagaggacgtttcctgtttagggaaagaaaaaacatcttcagatcataggtcctaaaaatacgggcaagctcttaactatttaaaaatgaaattacaggcccgggcacggtggctcacacctgtaatcccagcactttgggaggctgaggcaggcagatcatgaggtcaagagatcgagaccagcctggccaacgtggtgaaaccccatctctactaaaaatacaaaaattagccgggtagtaggtaggcgcgcgcctgttgtcttagctactcaggaggctgaggcaggagaatcgcttgaaaacaggaggtggaggttgcagtgagccgagatcacgccactgcactccagcctggtgacagcgtgagactctttaaaaaaagaaattaaaagagttgagacaaacgtttcctacattcttttccatgtgtaaaatcatgaaaaagcctgtcaccggacttgcattggatgagatgagtcagaccaaaacagtggccacccgtcttcctcctgtgagcctaagtgcagccgtgctagctgcgcaccgtggctaaggatgacgtctgtgttcctgtccatcactgatgctgctggctactgcatgtgccacacctgtctgttcgccattcctaacattctgtttcattcttcctcgggagatatttcaaacatttggtcttttcttttaacactgagggtaggcccttaggaaatttatttaggaaagtctgaacacgttatcacttggttttctggaaagtagcttaccctagaaaacagctgcaaatgccagaaagatgatccctaaaaatgttgagggacttctgttcattcatcccgagaacattggcttccacatcacagtatctacccttacatggtttaggattaaagccaggcaatcttttactatg

SEQ ID NO: 41 - DNA, 1296 bp, Homo sapiens
gcttttgtcccccgcccgccgcttctgtccgagaggccgcccgcgaggcgcatcctgaccgcgagcgtcgggtcccagagccgggcgcggctggggcccgaggctagcatctctcgggagccgcaaggcgagagctgcaaagtttaattagacacttcagaattttgatcacctaatgttgatttcagatgtaaaagtcaagagaagactctaaaaatagcaaagatgcttttgagccagaatgccttcatcgtcagatcacttaatttggttctcatggtgtatatcagcctcgtgtttggtatttcatatgattcgcctgattacacagatgaatcttgcactttcaagatatcattgcgaaatttccggtccatcttatcatgggaattaaaaaaccactccattgtaccaactcactatacattgctgtatacaatcatgagtaaaccagaagatttgaaggtggttaagaactgtgcaaataccacaagatcattttgtgacctcacagatgagtggagaagcacacacgaggcctatgtcaccgtcctagaaggattcagcgggaacacaacgttgttcagttgctcacacaatttctggctggccatagacatgtcttttgaaccaccagagtttgagattgttggttttaccaaccacattaatgtgatggtgaaatttccatctattgttgaggaagaattacagtttgatttatctctcgtcattgaagaacagtcagagggaattgttaagaagcataaacccgaaataaaaggaaacatgagtggaaatttcacctatatcattgacaagttaattccaaacacgaactactgtgtatctgtttatttagagcacagtgatgagcaagcagtaataaagtctcccttaaaatgcaccctccttccacctggccaggaatcagaatcagcagaatctgccaaaataggaggaataattactgtgtttttgatagcattggtcttgacaagcaccatagtgacactgaaatggattggttatatatgcttaagaaatagcctccccaaagtcttgaggcaaggtctcactaagggctggaatgcagtggctattcacaggtgcagtcataatgcactacagtctgaaactcctgagctcaaacagtcgtcctgcctaagcttccccagtagctgggattacaagcgtgcatccctgtgccccagtgattaagttttattatgtagaaaataaagagcaaacagttacaaaagaaaaaaaaaaaaaaaaaaaaaaaaaaaaaaaaaa

SEQ ID NO: 42 - DNA, 725 bp, Homo sapiens
cttcagaagcaatgggaaaaatcagcagtcttccaacccaattatttaagtgctgcttttgtgatttcttgaaggtgaagatgcacaccatgtcctcctcgcatctcttctacctggcgctgtgcctgctcaccttcaccagctctgccacggctggaccggagacgctctgcggggctgagctggtggatgctcttcagttcgtgtgtggagacaggggcttttatttcaacaagcccacagggtatggctccagcagtcggagggcgcctcagacaggtatcgtggatgagtgctgcttccggagctgtgatctaaggaggctggagatgtattgcgcacccctcaagcctgccaagtcagctcgctctgtccgtgcccagcgccacaccgacatgcccaagacccagaaggaagtacatttgaagaacgcaagtagagggagtgcaggaaacaagaactacaggatgtaggaagaccctcctgaggagtgaagagtgacatgccaccgcaggatcctttgctctgcacgagttacctgttaaactttggaacacctaccaaaaaataagtttgataacatttaaaagatgggcgtttcccccaatgaaatacacaagtaaacattccaacattgtctttaggagtgatttgcaccttgcaaaaatggtcctggagttggtagattgctgttgatcttttatcaataatgttctatagaaaag

SEQ ID NO: 43 - DNA, 3632 bp, Homo sapiens
aaagagctggaggcgcgcaggccggctccgctccggccccggacgatgcggcgcgcccaggatgctgccgtgcctcgtagtgctgctggcggcgctcctcagcctccgtcttggctcagacgctcatgggacagagctgcccagccctccgtctgtgtggtttgaagcagaatttttccaccacatcctccactggacacccatcccaaatcagtctgaaagtacctgctatgaagtggcgctcctgaggtatggaatagagtcctggaactccatctccaactgtagccagaccctgtcctatgaccttaccgcagtgaccttggacctgtaccacagcaatggctaccgggccagagtgcgggctgtggacggcagccggcactccaactggaccgtcaccaacacccgcttctctgtggatgaagtgactctgacagttggcagtgtgaacctagagatccacaatggcttcatcctcgggaagattcagctacccaggcccaagatggcccccgcgaatgacacatatgaaagcatcttcagtcacttccgagagtatgagattgccattcgcaaggtgccgggaaacttcacgttcacacacaagaaagtaaaacatgaaaacttcagcctcctaacctctggagaagtgggagagttctgtgtccaggtgaaaccatctgtcgcttcccgaagtaacaaggggatgtggtctaaagaggagtgcatctccctcaccaggcagtatttcaccgtgaccaacgtcatcatcttctttgcctttgtcctgctgctctccggagccctcgcctactgcctggccctccagctgtatgtgcggcgccgaaagaagctacccagtgtcctgctcttcaagaagcccagccccttcatcttcatcagccagcgtccctccccagagacccaagacaccatccacccgcttgatgaggaggcctttttgaaggtgtccccagagctgaagaacttggacctgcacggcagcacagacagtggctttggcagcaccaagccatccctgcagactgaagagccccagttcctcctccctgaccctcacccccaggctgacagaacgctgggaaacggggagccccctgtgctgggggacagctgcagtagtggcagcagcaatagcacagacagcgggatctgcctgcaggagcccagcctgagccccagcacagggcccacctgggagcaacaggtggggagcaacagcaggggccaggatgacagtggcattgacttagttcaaaactctgagggccgggctggggacacacagggtggctcggccttgggccaccacagtcccccggagcctgaggtgcctggggaagaagacccagctgctgtggcattccagggttacctgaggcagaccagatgtgctgaagagaaggcaaccaagacaggctgcctggaggaagaatcgcccttgacagatggccttggccccaaattcgggagatgcctggttgatgaggcaggcttgcatccaccagccctggccaagggctatttgaaacaggatcctctagaaatgactctggcttcctcaggggccccaacgggacagtggaaccagcccactgaggaatggtcactcctggccttgagcagctgcagtgacctgggaatatctgactggagctttgcccatgaccttgcccctctaggctgtgtggcagccccaggtggtctcctgggcagctttaactcagacctggtcaccctgcccctcatctctagcctgcagtcaagtgagtgactcgggctgagaggctgcttttgattttagccatgcctgctcctctgcctggaccaggaggagggccctggggcagaagttaggcacgaggcagtctgggcacttttctgcaagtccactggggctggcccagccaggctgcagggctggtcagggtgtctggggcaggaggaggccaactcactgaactagtgcagggtatgtgggtggcactgacctgttctgttgactggggccctgcagactctggcagagctgagaagggcagggaccttctccctcctaggaactctttcctgtatcataaaggattatttgctcaggggaaccatggggctttctggagttgtggtgaggccaccaggctgaagtcagctcagacccagacctccctgcttaggccactcgagcatcagagcttccagcaggaggaagggctgtaggaatggaagcttcagggccttgctgctggggtcatttttaggggaaaaaggaggatatgatggtcacatggggaacctcccctcatcgggcctctggggcaggaagcttgtcactggaagatcttaaggtatatattttctggacactcaaacacatcataatggattcactgaggggagacaaagggagccgagaccctggatggggcttccagctcagaacccatccctctggtgggtacctctggcacccatctgcaaatatctccctctctccaacaaatggagtagcatccccctggggcacttgctgaggccaagccactcacatcctcactttgctgccccaccatcttgctgacaacttccagagaagccatggttttttgtattggtcataactcagccctttgggcggcctctgggcttgggcaccagctcatgccagccccagagggtcagggttggaggcctgtgcttgtgtttgctgctaatgtccagctacagacccagaggataagccactgggcactgggctggggtccctgccttgttggtgttcagctgtgtgattttggactagccacttgtcagagggcctcaatctcccatctgtgaaataaggactccacctttaggggaccctccatgtttgctgggtattagccaagctggtcctgggagaatgcagatactgtccgtggactaccaagctggcttgtttcttatgccagaggctaacagatccaatgggagtccatggtgtcatgccaagacagtatcagacacagccccagaagggggcattatgggccctgcctccccataggccatttggactctgccttcaaacaaaggcagttcagtccacaggcatggaagctgtgaggggacaggcctgtgcgtgccatccagagtcatctcagccctgcctttctctggagcattctgaaaacagatattctggcccagggaatccagccatgacccccacccctctgccaaagtactcttaggtgccagtctggtaactgaactccctctggaggcaggcttgagggaggattcctcagggttcccttgaaagctttatttatttattttgttcatttatttattggagaggcagcattgcacagtgaaagaattctggatatctcaggagccccgaaattctagctctgactttgctgtttccagtggtatgaccttggagaagtcacttatcctcttggagcctcagtttcctcatctgcagaataatgactgacttgtctaattcatagggatgtgaggttctgctgaggaaatgggtatgaatgtgccttgaacacaaagctctgtcaataagtgatacatgttttttattccaataaattgtcaagaccaca

SEQ ID NO: 44 - DNA, 2002 bp, Homo sapiens
atggccaaagttccagacatgtttgaagacctgaagaactgttacagtgaaaatgaagaagacagttcctccattgatcatctgtctctgaatcagaaatccttctatcatgtaagctatggcccactccatgaaggctgcatggatcaatctgtgtctctgagtatctctgaaacctctaaaacatccaagcttaccttcaaggagagcatggtggtagtagcaaccaacgggaaggttctgaagaagagacggttgagtttaagccaatccatcactgatgatgacctggaggccatcgccaatgactcagaggaagaaatcatcaagcctaggtcagcaccttttagcttcctgagcaatgtgaaatacaactttatgaggatcatcaaatacgaattcatcctgaatgacgccctcaatcaaagtataattcgagccaatgatcagtacctcacggctgctgcattacataatctggatgaagcagtgaaatttgacatgggtgcttataagtcatcaaaggatgatgctaaaattaccgtgattctaagaatctcaaaaactcaattgtatgtgactgcccaagatgaagaccaaccagtgctgctgaaggagatgcctgagatacccaaaaccatcacaggtagtgagaccaacctcctcttcttctgggaaactcacggcactaagaactatttcacatcagttgcccatccaaacttgtttattgccacaaagcaagactactgggtgtgcttggcaggggggccaccctctatcactgactttcagatactggaaaaccaggcgtaggtctggagtctcacttgtctcacttgtgcagtgttgacagttcatatgtaccatgtacatgaagaagctaaatcctttactgttagtcatttgctgagcatgtactgagccttgtaattctaaatgaatgtttacactctttgtaagagtggaaccaacactaacatataatgttgttatttaaagaacaccctatattttgcatagtaccaatcattttaattattattcttcataacaattttaggaggaccagagctactgactatggctaccaaaaagactctacccatattacagatgggcaaattaaggcataagaaaactaagaaatatgcacaatagcagttgaaacaagaagccacagacctaggatttcatgatttcatttcaactgtttgccttctgcttttaagttgctgatgaactcttaatcaaatagcataagtttctgggacctcagttttatcattttcaaaatggagggaataatacctaagccttcctgccgcaacagttttttatgctaatcagggaggtcattttggtaaaatacttctcgaagccgagcctcaagatgaaggcaaagcacgaaatgttattttttaattattatttatatatgtatttataaatatatttaagataattataatatactatatttatgggaaccccttcatcctctgagtgtgaccaggcatcctccacaatagcagacagtgttttctgggataagtaagtttgatttcattaatacagggcattttggtccaagttgtgcttatcccatagccaggaaactctgcattctagtacttgggagacctgtaatcatataataaatgtacattaattaccttgagccagtaattggtccgatctttgactcttttgccattaaacttacctgggcattcttgtttcattcaattccacctgcaatcaagtcctacaagctaaaattagatgaactcaactttgacaaccatagaccactgttatcaaaactttcttttctggaatgtaatcaatgtttcttctaggttctaaaaattgtgatcagaccataatgttacattattatcaacaatagtgattgatagagtgttatcagtcataactaaataaagcttgcaagtgagggagtcatttcattggcgtttgagtcagcaaagaagtcaag

SEQ ID NO: 45 - DNA, 1496 bp, Homo sapiens
acaaaccttttcgaggcaaaaggcaaaaaaggctgctctgggattctcttcagccaatcttcaatgctcaagtgtctgaagcagccatggcagaagtacctaagctcgccagtgaaatgatggcttattacagtggcaatgaggatgacttgttctttgaagctgatggccctaaacagatgaagtgctccttccaggacctggacctctgccctctggatggcggcatccagctacgaatctccgaccaccactacagcaagggcttcaggcaggccgcgtcagttgttgtggccatggacaagctgaggaagatgctggttccctgcccacagaccttccaggagaatgacctgagcaccttctttcccttcatctttgaagaagaacctatcttcttcgacacatgggataacgaggcttatgtgcacgatgcacctgtacgatcactgaactgcacgctccgggactcacagcaaaaaagcttggtgatgtctggtccatatgaactgaaagctctccacctccagggacaggatatggagcaacaagtggtgttctccatgtcctttgtacaaggagaagaaagtaatgacaaaatacctgtggccttgggcctcaaggaaaagaatctgtacctgtcctgcgtgttgaaagatgataagcccactctacagctggagagtgtagatcccaaaaattacccaaagaagaagatggaaaagcgatttgtcttcaacaagatagaaatcaataacaagctggaatttgagtctgcccagttccccaactggtacatcagcacctctcaagcagaaaacatgcccgtcttcctgggagggaccaaaggcggccaggatataactgacttcaccatgcaatttgtgtcttcctaaagagagctgtacccagagagtcctgtgctgaatgtggactcaatccctagggctggcagaaagggaacagaaaggtttttgagtacggctatagcctggactttcctgttgtctacaccaatgcccaactgcctgccttagggtagtgctaagaggatctcctgtccatcagccaggacagtcagctctctcctttcagggccaatcccagcccttttgttgagccaggcctctctcacctctcctactcacttaaagcccgcctgacagaaaccaggccacattttggttctaagaaaccctcctctgtcattcgctcccacattctgatgagcaaccgcttccctatttatttatttatttgtttgtttgttttgattcattggtctaatttattcaaagggggcaagaagtagcagtgtctgtaaaagagcctagtttttaatagctatggaatcaattcaatttggactggtgtgctctctttaaatcaagtcctttaattaagactgaaaatatataagctcagattatttaaatgggaatatttataaatgagcaaatatcatactgttcaatggttctcaaataaacttcact

SEQ ID NO: 46 - DNA, 1740 bp, Homo sapiens
tctcaaaggatgacacttctgtggtgtgtagtgagtctctacttttatggaatcctgcaaagtgatgcctcagaacgctgcgatgactggggactagacaccatgaggcaaatccaagtgtttgaagatgagccagctcgcatcaagtgcccactctttgaacacttcttgaaattcaactacagcacagcccattcagctggccttactctgatctggtattggactaggcaggaccgggaccttgaggagccaattaacttccgcctccccgagaaccgcattagtaaggagaaagatgtgctgtggttccggcccactctcctcaatgacactggcaactatacctgcatgttaaggaacactacatattgcagcaaagttgcatttcccttggaagttgttcaaaaagacagctgtttcaattcccccatgaaactcccagtgcataaactgtatatagaatatggcattcagaggatcacttgtccaaatgtagatggatattttccttccagtgtcaaaccgactatcacttggtatatgggctgttataaaatacagaattttaataatgtaatacccgaaggtatgaacttgagtttcctcattgccttaatttcaaataatggaaattacacatgtgttgttacatatccagaaaatggacgtacgtttcatctcaccaggactctgactgtaaaggtagtaggctctccaaaaaatgcagtgccccctgtgatccattcacctaatgatcatgtggtctatgagaaagaaccaggagaggagctactcattccctgtacggtctattttagttttctgatggattctcgcaatgaggtttggtggaccattgatggaaaaaaacctgatgacatcactattgatgtcaccattaacgaaagtataagtcatagtagaacagaagatgaaacaagaactcagattttgagcatcaagaaagttacctctgaggatctcaagcgcagctatgtctgtcatgctagaagtgccaaaggcgaagttgccaaagcagccaaggtgaagcagaaagtgccagctccaagatacacagtggaactggcttgtggttttggagccacagtcctgctagtggtgattctcattgttgtttaccatgtttactggctagagatggtcctattttaccgggctcattttggaacagatgaaaccattttagatggaaaagagtatgatatttatgtatcctatgcaaggaatgcggaagaagaagaatttgtattactgaccctccgtggagttttggagaatgaatttggatacaagctgtgcatctttgaccgagacagtctgcctgggggaattgtcacagatgagactttgagcttcattcagaaaagcagacgcctcctggttgttctaagccccaactacgtgctccagggaacccaagccctcctggagctcaaggctggcctagaaaatatggcctctcggggcaacatcaacgtcattttagtacagtacaaagctgtgaaggaaacgaaggtgaaagagctgaagagggctaagacggtgctcacggtcattaaatggaaaggggaaaaatccaagtatccacagggcaggttctggaagcagctgcaggtggccatgccagtgaagaaaagtcccaggcggtctagcagtgatgagcagggcctctcgtattcatctttgaaaaatgtatgaaaggaataatgaaaagga

SEQ ID NO: 47 - DNA, 4906 bp, Homo sapiens
gttgctgtcgccgttgctgtcggccgaggccaccaagaggcaacgggcggcaggttgcagtggaggggcctccgctcccctcggtggtgtgtgggtcctgggggtgcctgccggcccagccgaggaggcccacgcccaccatggtcccctgctggaaccatggcaacatcacccgctccaaggcggaggagctgctttccaggacaggcaaggacgggagcttcctcgtgcgtgccagcgagtccatctcccgggcatacgcgctctgcgtgctgtatcggaattgcgtttacacttacagaattctgcccaatgaagatgataaattcactgttcaggcatccgaaggcgtctccatgaggttcttcaccaagctggaccagctcatcgagttttacaagaaggaaaacatggggctggtgacccatctgcaataccctgtgccgctggaggaagaggacacaggcgacgaccctgaggaggacacagaaagtgtcgtgtctccacccgagctgcccccaagaaacatcccgctgactgccagctcctgtgaggccaaggaggttcctttttcaaacgagaatccccgagcgaccgagaccagccggccgagcctctccgagacattgttccagcgactgcaaagcatggacaccagtgggcttccagaagagcatcttaaggccatccaagattatttaagcactcagctcgcccaggactctgaatttgtgaagacagggtccagcagtcttcctcacctgaagaaactgaccacactgctctgcaaggagctctatggagaagtcatccggaccctcccatccctggagtctctgcagaggttatttgaccagcagctctccccgggcctccgtccacgtcctcaggttcctggtgaggccaatcccatcaacatggtgtccaagctcagccaactgacaagcctgttgtcgtccattgaagacaaggtcaaggccttgctgcacgagggtcctgagtctccgcaccggccctcccttatccctccagtcacctttgaggtgaaggcagagtctctggggattcctcagaaaatgcagctcaaagtcgacgttgagtctgggaaactgatcattaagaagtccaaggatggttctgaggacaagttctacagccacaagaaaatcctgcagctgattaagtcacagaaatttctgaataagttggtgatcttggtggaaacggagaaggagaagatcctgcggaaggaatatgtttttgctgactccaaaaagagagaaggcttctgccagctcctgcagcagatgaagaacaagcactcagagcagccggagcccgacatgatcaccatcttcatcggcacctggaacatgggtaacgccccccctcccaagaagatcacgtcctggtttctctccaaggggcagggaaagacgcgggacgactctgcggactacatcccccatgacatttacgtgatcggcacccaagaggaccccctgagtgagaaggagtggctggagatcctcaaacactccctgcaagaaatcaccagtgtgacttttaaaacagtcgccatccacacgctctggaacatccgcatcgtggtgctggccaagcctgagcacgagaaccggatcagccacatctgtactgacaacgtgaagacaggcattgcaaacacactggggaacaagggagccgtgggggtgtcgttcatgttcaatggaacctccttagggttcgtcaacagccacttgacttcaggaagtgaaaagaaactcaggcgaaaccaaaactatatgaacattctccggttcctggccctgggcgacaagaagctgagtccctttaacatcactcaccgcttcacgcacctcttctggtttggggatcttaactaccgtgtggatctgcctacctgggaggcagaaaccatcatccagaaaatcaagcagcagcagtacgcagacctcctgtcccacgaccagctgctcacagagaggagggagcagaaggtcttcctacacttcgaggaggaagaaatcacgtttgccccaacctaccgttttgagagactgactcgggacaaatacgcctacaccaagcagaaagcgacagggatgaagtacaacttgccttcctggtgtgaccgagtcctctggaagtcttatcccctggtgcacgtggtgtgtcagtcttatggcagtaccagcgacatcatgacgagtgaccacagccctgtctttgccacatttgaggcaggagtcacttcccagtttgtctccaagaacggtcccgggactgttgacagccaaggacagattgagtttctcaggtgctatgccacattgaagaccaagtcccagaccaaattctacctggagttccactcgagctgcttggagagttttgtcaagagtcaggaaggagaaaatgaagaaggaagtgagggggagctggtggtgaagtttggtgagactcttccaaagctgaagcccattatctctgaccctgagtacctgctagaccagcacatcctcatcagcatcaagtcctctgacagcgacgaatcctatggcgagggctgcattgcccttcggttagaggccacagaaacgcagctgcccatctacacgcctctcacccaccatggggagttgacaggccacttccagggggagatcaagctgcagacctctcagggcaagacgagggagaagctctatgactttgtgaagacggagcgtgatgaatccagtgggccaaagaccctgaagagcctcaccagccacgaccccatgaagcagtgggaagtcactagcagggcccctccgtgcagtggctccagcatcactgaaatcatcaaccccaactacatgggagtggggccctttgggccaccaatgcccctgcacgtgaagcagaccttgtcccctgaccagcagcccacagcctggagctacgaccagccgcccaaggactccccgctggggccctgcaggggagaaagtcctccgacacctcccggccagccgcccatatcacccaagaagtttttaccctcaacagcaaaccggggtctccctcccaggacacaggagtcaaggcccagtgacctggggaagaacgcaggggacacgctgcctcaggaggacctgccgctgacgaagcccgagatgtttgagaaccccctgtatgggtccctgagttccttccctaagcctgctcccaggaaggaccaggaatcccccaaaatgccgcggaaggaacccccgccctgcccggaacccggcatcttgtcgcccagcatcgtgctcaccaaagcccaggaggctgatcgcggcgaggggcccggcaagcaggtgcccgcgccccggctgcgctccttcacgtgctcatcctctgccgagggcagggcggccggcggggacaagagccaagggaagcccaagaccccggtcagctcccaggccccggtgccggccaagaggcccatcaagccttccagatcggaaatcaaccagcagaccccgcccaccccgacgccgcggccgccgctgccagtcaagagcccggcggtgctgcacctccagcactccaagggccgcgactaccgcgacaacaccgagctcccgtatcacggcaagcaccggccggaggaggggccaccagggcctctaggcaggactgccatgcagtgaagccctcagtgagctgccactgagtcgggagcccagaggaacggcgtgaagccactggaccctctcccgggacctcctgctggctcctcctgcccagcttcctatgcaaggctttgtgttttcaggaaagggcctagcttctgtgtggcccacagagttcactgcctgtgagacttagcaccaagtgctgaggctggaagaaaaacgcacaccagacgggcaacaaacagtctgggtccccagctcgctcttggtacttgggaccccagtgccttgttgagggcgccattctgaagaaaggaactgcagcgccgatttgagggtggagatatagataataataatattaataataataatggccacatggatcgaacactcatggtgtgccaagtgctgtgctaagtgctttacgaacattcgtcatatcaggatgacctcgagagctgaggctctagcacctaaaaccacgtgcccaaacccaccagtttaaaacggtgtgtgttcggaggggtgaaagcattaagaagcccagtgccctcctggagtgagacaagggctcggccttaaggagctgaagagtctgggtagcttgtttagggtacaagaagcctgttctgtccagcttcagtgacacaagctgctttagctaaagtcccgcgggttccggcatggctaggctgagagcagggatctacctggcttctcagttctttggttggaaggagcaggaaatcagctcctattctccagtggagagatctggcctcagcttgggctagagatgccaaggcctgtgccaggttccctgtgccctcctcgaggtgggcagccatcaccagccacagttaagccaagccccccaacatgtattccatcgtgctggtagaagagtctttgctgttgctcccgaaagccgtgctctccagcctggctgccagggagggtgggcctcttggttccaggctcttgaaatagtgcagccttttcttcctatctctgtggctttcagctctgcttccttggttattaggagaatagatgggtgatgtctttccttatgttgctttttcaacatagcagaattaatgtagggagctaaatccagtggtgtgtgtgaatgcagaagggaatgcaccccacattcccatgatggaagtctgcgtaaccaataaattgtgcctttctcactcaaaacc

SEQ ID NO: 48 - DNA, 4742 bp, Homo sapiens
ttttctgcccttctttgctttggtggcttccttgtggttcctcagtggtgcctgcaacccctggttcacctccttccaggttctggctccttccagccatggctctcagagtccttctgttaacagccttgaccttatgtcatgggttcaacttggacactgaaaacgcaatgaccttccaagagaacgcaaggggcttcgggcagagcgtggtccagcttcagggatccagggtggtggttggagccccccaggagatagtggctgccaaccaaaggggcagcctctaccagtgcgactacagcacaggctcatgcgagcccatccgcctgcaggtccccgtggaggccgtgaacatgtccctgggcctgtccctggcagccaccaccagcccccctcagctgctggcctgtggtcccaccgtgcaccagacttgcagtgagaacacgtatgtgaaagggctctgcttcctgtttggatccaacctacggcagcagccccagaagttcccagaggccctccgagggtgtcctcaagaggatagtgacattgccttcttgattgatggctctggtagcatcatcccacatgactttcggcggatgaaggagtttgtctcaactgtgatggagcaattaaaaaagtccaaaaccttgttctctttgatgcagtactctgaagaattccggattcactttaccttcaaagagttccagaacaaccctaacccaagatcactggtgaagccaataacgcagctgcttgggcggacacacacggccacgggcatccgcaaagtggtacgagagctgtttaacatcaccaacggagcccgaaagaatgcctttaagatcctagttgtcatcacggatggagaaaagtttggcgatcccttgggatatgaggatgtcatccctgaggcagacagagagggagtcattcgctacgtcattggggtgggagatgccttccgcagtgagaaatcccgccaagagcttaataccatcgcatccaagccgcctcgtgatcacgtgttccaggtgaataactttgaggctctgaagaccattcagaaccagcttcgggagaagatctttgcgatcgagggtactcagacaggaagtagcagctcctttgagcatgagatgtctcaggaaggcttcagcgctgccatcacctctaatggccccttgctgagcactgtggggagctatgactgggctggtggagtctttctatatacatcaaaggagaaaagcaccttcatcaacatgaccagagtggattcagacatgaatgatgcttacttgggttatgctgccgccatcatcttacggaaccgggtgcaaagcctggttctgggggcacctcgatatcagcacatcggcctggtagcgatgttcaggcagaacactggcatgtgggagtccaacgctaatgtcaagggcacccagatcggcgcctacttcggggcctccctctgctccgtggacgtggacagcaacggcagcaccgacctggtcctcatcggggccccccattactacgagcagacccgagggggccaggtgtccgtgtgccccttgcccagggggagggctcggtggcagtgtgatgctgttctctacggggagcagggccaaccctggggccgctttggggcagccctaacagtgctgggggacgtaaatggggacaagctgacggacgtggccattggggccccaggagaggaggacaaccggggtgctgtttacctgtttcacggaacctcaggatctggcatcagcccctcccatagccagcggatagcaggctccaagctctctcccaggctccagtattttggtcagtcactgagtgggggccaggacctcacaatggatggactggtagacctgactgtaggagcccaggggcacgtgctgctgctcaggtcccagccagtactgagagtcaaggcaatcatggagttcaatcccagggaagtggcaaggaatgtatttgagtgtaatgatcaggtggtgaaaggcaaggaagccggagaggtcagagtctgcctccatgtccagaagagcacacgggatcggctaagagaaggacagatccagagtgttgtgacttatgacctggctctggactccggccgcccacattcccgcgccgtcttcaatgagacaaagaacagcacacgcagacagacacaggtcttggggctgacccagacttgtgagaccctgaaactacagttgccgaattgcatcgaggacccagtgagccccattgtgctgcgcctgaacttctctctggtgggaacgccattgtctgctttcgggaacctccggccagtgctggcggaggatgctcagagactcttcacagccttgtttccctttgagaagaattgtggcaatgacaacatctgccaggatgacctcagcatcaccttcagtttcatgagcctggactgcctcgtggtgggtgggccccgggagttcaacgtgacagtgactgtgagaaatgatggtgaggactcctacaggacacaggtcaccttcttcttcccgcttgacctgtcctaccggaaggtgtccacgctccagaaccagcgctcacagcgatcctggcgcctggcctgtgagtctgcctcctccaccgaagtgtctggggccttgaagagcaccagctgcagcataaaccaccccatcttcccggaaaactcagaggtcacctttaatatcacgtttgatgtagactctaaggcttcccttggaaacaaactgctcctcaaggccaatgtgaccagtgagaacaacatgcccagaaccaacaaaaccgaattccaactggagctgccggtgaaatatgctgtctacatggtggtcaccagccatggggtctccactaaatatctcaacttcacggcctcagagaataccagtcgggtcatgcagcatcaatatcaggtcagcaacctggggcagaggagcctccccatcagcctggtgttcttggtgcccgtccggctgaaccagactgtcatatgggaccgcccccaggtcaccttctccgagaacctctcgagtacgtgccacaccaaggagcgcttgccctctcactccgactttctggctgagcttcggaaggcccccgtggtgaactgctccatcgctgtctgccagagaatccagtgtgacatcccgttctttggcatccaggaagaattcaatgctaccctcaaaggcaacctctcgtttgactggtacatcaagacctcgcataaccacctcctgatcgtgagcacagctgagatcttgtttaacgattccgtgttcaccctgctgccgggacagggggcgtttgtgaggtcccagacggagaccaaagtggagccgttcgaggtccccaaccccctgccgctcatcgtgggcagctctgtcgggggactgctgctcctggccctcatcaccgccgcgctgtacaagctcggcttcttcaagcggcaatacaaggacatgatgagtgaagggggtcccccgggggccgaaccccagtagcggctccttcccgacagagctgcctctcggtggccagcaggactctgcccagaccacacgtagcccccaggctgctggacacgtcggacagcgaagtatccccgacaggacgggcttgggcttccatttgtgtgtgtgcaagtgtgtatgtgcgtgtgtgcaagtgtctgtgtgcaagtgtgtgcacatgtgtgcgtgtgcgtgcatgtgcacttgcacgcccatgtgtgagtgtgtgcaagtatgtgagtgtgtccaagtgtgtgtgcgtgtgtccatgtgtgtgcaagtgtgtgcatgtgtgcgagtgtgtgcatgtgtgtgctcaggggcgtgtggctcacgtgtgtgactcagatgtctctggcgtgtgggtaggtgacggcagcgtagcctctccggcagaagggaactgcctgggctcccttgtgcgtgggtgaagccgctgctgggttttcctccgggagaggggacggtcaatcctgtgggtgaagacagagggaaacacagcagcttctctccactgaaagaagtgggacttcccgtcgcctgcgagcctgcggcctgctggagcctgcgcagcttggatggagactccatgagaagccgtgggtggaaccaggaacctcctccacaccagcgctgatgcccaataaagatgcccactgaggaatgatgaagcttcctttctggattcatttattatttcaatgtgactttaattttttggatggataagcttgtctatggtacaaaaatcacaaggcattcaagtgtacagtgaaaagtctccctttccagatattcaagtcacctccttaaaggtagtcaagattgtgttttgaggtttccttcagacagattccaggcgatgtgcaagtgtatgcacgtgtgcacacacaccacacatacacacacacaagcttttttacacaaatggtagcatactttatattggtctgtatcttgctttttttcaccaatatttctcagacatcggttcatattaagacataaattactttttcattcttttataccgctgcatagtattccattgtgtgagtgtaccataatgtatttaaccagtcttcttttgatatactattttcattctcttgttattgcatcaatgctgagttaataaatcaaatatatgtcatttttgcatatatgtaaggataa

SEQ ID NO: 49 - DNA, 4654 bp, Homo sapiens
gaattcctgccactcttcctgcaacggcccaggagctcagagctccacatctgaccttctagtcatgaccaggaccagggcagcactcctcctgttcacagccttagcaacttctctaggtttcaacttggacacagaggagctgacagccttccgtgtggacagcgctgggtttggagacagcgtggtccagtatgccaactcctgggtggtggttggagccccccaaaagataacagctgccaaccaaacgggtggcctctaccagtgtggctacagcactggtgcctgtgagcccatcggcctgcaggtgcccccggaggccgtgaacatgtccctgggcctgtccctggcgtctaccaccagcccttcccagctgctggcctgcggccccaccgtgcaccacgagtgcgggaggaacatgtacctcaccggactctgcttcctcctgggccccacccagctcacccagaggctcccggtgtccaggcaggagtgcccaagacaggagcaggacattgtgttcctgatcgatggctcaggcagcatctcctcccgcaactttgccacgatgatgaacttcgtgagagctgtgataagccagttccagagacccagcacccagttttccctgatgcagttctccaacaaattccaaacacacttcactttcgaggaattcaggcgcacgtcaaaccccctcagcctgttggcttctgttcaccagctgcaagggtttacatacacggccaccgccatccaaaatgtcgtgcaccgattgttccatgcctcatatggggcccgtagggatgccaccaaaattctcattgtcatcactgatgggaagaaagaaggcgacagcctggattataaggatgtcatccccatggctgatgcagcaggcatcatccgctatgcaattggggttggattagcttttcaaaacagaaattcttggaaagaattaaatgacattgcatcgaagccctcccaggaacacatatttaaagtggaggactttgatgctctgaaagatattcaaaaccaactgaaggagaagatctttgccattgagggtacggagaccacaagcagtagctccttcgaattggagatggcacaggagggcttcagcgctgtgttcacacctgatggccccgttctgggggctgtggggagcttcacctggtctggaggtgccttcctgtaccccccaaatatgagccctaccttcatcaacatgtctcaggagaatgtggacatgagggactcttacctgggttactccaccgagctggccctctggaaaggggtgcagagcctggtcctgggggccccccgctaccagcacaccgggaaggctgtcatcttcacccaggtgtccaggcaatggaggatgaaggccgaagtcacggggactcagatcggctcctacttcggggcctccctctgctccgtggacgtagacaccgacggcagcaccgacctggtcctcatcggggccccccattactacgagcagacccgagggggccaggtgtctgtgtgtcccttgcccagggggtggagaaggtggtggtgtgatgctgttctctacggggagcagggccacccctggggtcgctttggggcggctctgacagtgctgggggatgtgaatggggacaagctgacagacgtggtcatcggggccccaggagaggaggagaaccggggtgctgtctacctgtttcacggagtcttgggacccagcatcagcccctcccacagccagcggatcgcgggctcccagctctcctccaggctgcagtattttgggcaggcactgagcgggggtcaagacctcacccaggatggactggtggacctggctgtgggggcccggggccaggtgctcctgctcaggaccagacctgtgctctgggtgggggtgagcatgcagttcatacctgccgagatccccaggtctgcgtttgagtgtcgggagcaggtggtctctgagcagaccctggtacagtccaacatctgcctttacattgacaaacgttctaagaacctgcttgggagccgtgacctccaaagctctgtgaccttggacctggccctcgaccctggccgcctgagtccccgtgccaccttccaggaaacaaagaaccggagtctgagccgagtccgagtcctcgggctgaaggcacactgtgaaaacttcaacctgctgctcccgagctgcgtggaggactctgtgacccccattaccttgcgtctgaacttcacgctggtgggcaagcccctccttgccttcagaaacctgcggcctatgctggccgcactggctcagagatacttcacggcctccctaccctttgagaagaactgtggagccgaccatatctgccaggacaatctcggcatctccttcagcttcccaggcttgaagtccctgctggtggggagtaacctggagctgaacgcagaagtgatggtgtggaatgacggggaagactcctacggaaccaccatcaccttctcccaccccgcaggactgtcctaccgctacgtggcagagggccagaaacaagggcagctgcgttccctgcacctgacatgtgacagcgccccagttgggagccagggcacctggagcaccagctgcagaatcaaccacctcatcttccgtggcggcgcccagatcaccttcttggctacctttgacgtctcccccaaggctgtcctgggagaccggctgcttctgacagccaatgtgagcagtgagaacaacactcccaggaccagcaagaccaccttccagctggagctcccggtgaagtatgctgtctacactgtggttagcagccacgaacaattcaccaaatacctcaacttctcagagtctgaggagaaggaaagccatgtggccatgcacagataccaggtcaataacctgggacagagggacctgcctgtcagcatcaacttctgggtgcctgtggagctgaaccaggaggctgtgtggatggatgtggaggtctcccacccccagaacccatcccttcggtgctcctcagagaaaatcgcacccccagcatctgacttcctggcgcacattcagaagaatcccgtgctggactgctccattgctggctgcctgcggttccgctgtgacgtcccctccttcagcgtccaggaggagctggatttcaccctgaagggcaacctcagctttggctgggtccgccagatattgcagaagaaggtgtcggtcgtgagtgtggctgaaattacgttcgacacatccgtgtactcccagcttccaggacaggaggcatttatgagagctcagacgacaacggtgctggagaagtacaaggtccacaaccccacccccctcatcgtaggcagctccattgggggtctgttgctgctggcactcatcacagcggtactgtacaaagttggcttcttcaagcgtcagtacaaggaaatgatggaggaggcaaatggacaaattgccccagaaaacgggacacagacccccagcccgcccagtgagaaatgatccctctttgccttggacttcttctcccgcgattttccccacttacttaccctcacctgtcaggctgacggggaggaaccactgcaccaccgagagaggctgggatgggcctgcttcctgtctttgggagaaaacgtcttgcttgggaaggggcctttgtcttgtcaaggttccaactggaaacccttaggacagggtccctgctgtgttccccaaaaggacttgacttgcaatttctacctagaaatacatggacaatacccccaggcctcagtctcccttctcccatgaggcacgaatgatctttctttcctttccttttttttttttttcttttctttttttttttttttgagacggagtctcgctctgtcacccaggctggagtgcaatggcgtgatctcggctcgctgcaacctccgcctcccgggttcaagtaattctgctgtctcagcctcctgcgtagctgggactacaggcacacgccacctcgcccggcccgatctttctaaaatacagttctgaatatgctgctcatccccacctgtcttcaacagctccccattaccctcaggacaatgtctgaactctccagcttcgcgtgagaagtccccttccatcccagagggtgggcttcagggcgcacagcatgagagcctctgtgcccccatcaccctcgtttccagtgaattagtgtcatgtcagcatcagctcagggcttcatcgtggggctctcagttccgattccccaggctgaattgggagtgagatgcctgcatgctgggttctgcacagctggcctcccgcggttgggtcaacattgctggcctggaagggaggagcgccctctagggagggacatggccccggtgcggctgcagctcaccagccccaggggcagaagagacccaaccacttcctattttttgaggctatgaatatagtacctgaaaaaatgccaagcactagattatttttttaaaaagcgtactttaaatgtttgtgttaatacacattaaaacatcgcacaaaaacgatgcatctaccgctccttgggaaataatctgaaaggtctaaaaataaaaaagccttctgtgg

SEQ ID NO: 50 - DNA, 1603 bp, Homo sapiens
tgagatgagagctgccgacagttgggggtcaagggaggagacgccatgatccccaccttcacggctctgctctgcctcgggctgagtctgggccccaggacccacatgcaggcagggcccctccccaaacccaccctctgggctgagccaggctctgtgatcagctgggggaactctgtgaccatctggtgtcaggggaccctggaggctcgggagtaccgtctggataaagaggaaagcccagcaccctgggacagacagaacccactggagcccaagaacaaggccagattctccatcccatccatgacagaggactatgcagggagataccgctgttactatcgcagccctgtaggctggtcacagcccagtgaccccctggagctggtgatgacaggagcctacagtaaacccaccctttcagccctgccgagtcctcttgtgacctcaggaaagagcgtgaccctgctgtgtcagtcacggagcccaatggacactttccttctgatcaaggagcgggcagcccatcccctactgcatctgagatcagagcacggagctcagcagcaccaggctgaattccccatgagtcctgtgacctcagtgcacggggggacctacaggtgcttcagctcacacggcttctcccactacctgctgtcacaccccagtgaccccctggagctcatagtctcaggatccttggagggtcccaggccctcacccacaaggtccgtctcaacagctgcaggccctgaggaccagcccctcatgcctacagggtcagtcccccacagtggtctgagaaggcactgggaggtactgatcggggtcttggtggtctccatcctgcttctctccctcctcctcttcctcctcctccaacactggcgtcagggaaaacacaggacattggcccagagacaggctgatttccaacgtcctccaggggctgccgagccagagcccaaggacgggggcctacagaggaggtccagcccagctgctgacgtccagggagaaaacttctgtgctgccgtgaagaacacacagcctgaggacggggtggaaatggacactcggcagagcccacacgatgaagacccccaggcagtgacgtatgccaaggtgaaacactccagacctaggagagaaatggcctctcctccctccccactgtctggggaattcctggacacaaaggacagacaggcagaagaggacagacagatggacactgaggctgctgcatctgaagccccccaggatgtgacctacgcccagctgcacagctttaccctcagacagaaggcaactgagcctcctccatcccaggaaggggcctctccagctgagcccagtgtctatgccactctggccatccactaatccaggggggacccagaccccacaagccatggagactcaggaccccagaaggcatggaagctgcctccagtagacatcactgaaccccagccagcccagacccctgacacagaccactagaagattccgggaacgttgggagtcacctgattctgcaaagataaataatatccctgcattatcaaaataaagtagcagacctctcaattca

SEQ ID NO: 51 - DNA, 3549 bp, Homo sapiens
cccctcttcctcctcctcaagggaaagctgcccacttctagctgccctgccatcccctttaaagggcgacttgctcagcgccaaaccgcggctccagccctctccagcctccggctcagccggctcatcagtcggtccgcgccttgcagctcctccagagggacgcgccccgagatggagagcaaagccctgctcgtgctgactctggccgtgtggctccagagtctgaccgcctcccgcggaggggtggccgccgccgaccaaagaagagattttatcgacatcgaaagtaaatttgccctaaggacccctgaagacacagctgaggacacttgccacctcattcccggagtagcagagtccgtggctacctgtcatttcaatcacagcagcaaaaccttcatggtgatccatggctggacggtaacaggaatgtatgagagttgggtgccaaaacttgtggccgccctgtacaagagagaaccagactccaatgtcattgtggtggactggctgtcacgggctcaggagcattacccagtgtccgcgggctacaccaaactggtgggacaggatgtggcccggtttatcaactggatggaggaggagtttaactaccctctggacaatgtccatctcttgggatacagccttggagcccatgctgctggcattgcaggaagtctgaccaataagaaagtcaacagaattactggcctcgatccagctggacctaactttgagtatgcagaagccccgagtcgtctttctcctgatgatgcagattttgtagacgtcttacacacattcaccagagggtcccctggtcgaagcattggaatccagaaaccagttgggcatgttgacatttacccgaatggaggtacttttcagccaggatgtaacattggagaagctatccgcgtgattgcagagagaggacttggagatgtggaccagctagtgaagtgctcccacgagcgctccattcatctcttcatcgactctctgttgaatgaagaaaatccaagtaaggcctacaggtgcagttccaaggaagcctttgagaaagggctctgcttgagttgtagaaagaaccgctgcaacaatctgggctatgagatcaataaagtcagagccaaaagaagcagcaaaatgtacctgaagactcgttctcagatgccctacaaagtcttccattaccaagtaaagattcatttttctgggactgagagtgaaacccataccaatcaggcctttgagatttctctgtatggcaccgtggccgagagtgagaacatcccattcactctgcctgaagtttccacaaataagacctactccttcctaatttacacagaggtagatattggagaactactcatgttgaagctcaaatggaagagtgattcatactttagctggtcagactggtggagcagtcccggcttcgccattcagaagatcagagtaaaagcaggagagactcagaaaaaggtgatcttctgttctagggagaaagtgtctcatttgcagaaaggaaaggcacctgcggtatttgtgaaatgccatgacaagtctctgaataagaagtcaggctgaaactgggcgaatctacagaacaaagaacggcatgtgaattctgtgaagaatgaagtggaggaagtaacttttacaaaacatacccagtgtttggggtgtttcaaaagtggattttcctgaatattaatcccagccctacccttgttagttattttaggagacagtctcaagcactaaaaagtggctaattcaatttatggggtatagtggccaaatagcacatcctccaacgttaaaagacagtggatcatgaaaagtgctgttttgtcctttgagaaagaaataattgtttgagcgcagagtaaaataaggctccttcatgtggcgtattgggccatagcctataattggttagaacctcctattttaattggaattctggatctttcggactgaggccttctcaaactttactctaagtctccaagaatacagaaaatgcttttccgcggcacgaatcagactcatctacacagcagtatgaatgatgttttagaatgattccctcttgctattggaatgtggtccagacgtcaaccaggaacatgtaacttggagagggacgaagaaagggtctgataaacacagaggttttaaacagtccctaccattggcctgcatcatgacaaagttacaaattcaaggagatataaaatctagatcaattaattcttaataggctttatcgtttattgcttaatccctctctcccccttcttttttgtctcaagattatattataataatgttctctgggtaggtgttgaaaatgagcctgtaatcctcagctgacacataatttgaatggtgcagaaaaaaaaaagataccgtaattttattattagattctccaaatgattttcatcaatttaaaatcattcaatatctgacagttactcttcagttttaggcttaccttggtcatgcttcagttgtacttccagtgcgtctcttttgttcctggctttgacatgaaaagataggtttgagttcaaattttgcattgtgtgagcttctacagattttagacaaggaccgtttttactaagtaaaagggtggagaggttcctggggtggattcctaagcagtgcttgtaaaccatcgcgtgcaatgagccagatggagtaccatgagggttgttatttgttgtttttaacaactaatcaagagtgagtgaacaactatttataaactagatctcctatttttcagaatgctcttctacgtataaatatgaaatgataaagatgtcaaatatctcagaggctatagctgggaacccgactgtgaaagtatgtgatatctgaacacatactagaaagctctgcatgtgtgttgtccttcagcataattcggaagggaaaacagtcgatcaagggatgtattggaacatgtcggagtagaaattgttcctgatgtgccagaacttcgaccctttctctgagagagatgatcgtgcctataaatagtaggaccaatgttgtgattaacatcatcaggcttggaatgaattctctctaaaaataaaatgatgtatgatttgttgttggcatcccctttattaattcattaaatttctggatttgggttgtgacccagggtgcattaacttaaaagattcactaaagcagcacatagcactgggaactctggctccgaaaaactttgttatatatatcaaggatgttctggctttacattttatttattagctgtaaatacatgtgtggatgtgtaaatggagcttgtacatattggaaaggtcattgtggctatctgcatttataaatgtgtggtgctaactgtatgtgtctttatcagtgatggtctcacagagccaactcactcttatgaaatgggctttaacaaaacaagaaagaaacgtacttaactgtgtgaagaaatggaatcagcttttaataaaattgacaacattttattaccac

SEQ ID NO: 52 - DNA, 4077 bp, Homo sapiens
gaattcccagctctctgctcgctctgctcgcagtcacagacacttgagcacacgcgtacacccagacatcttcgggctgctattggattgactttgaaggttctgtgtgggtcgccgtggctgcatgtttgaatcaggtggagaagcacttcaacgctggacgaagtaaagattattgttgttattttttttttctctctctctctctcttaagaaaggaaaatatcccaaggactaatctgatcgggtcttccttcatcaggaacgaatgcaggaatttgggaactgagctgtgcaagtgctgaagaaggagatttgtttggaggaaacaggaaagagaaagaaaaggaaggaaaaaatacataatttcagggacgagagagagaagaaaaacggggactatggggagaaaaaagattcagattacgaggattatggatgaacgtaacagacaggtgacatttacaaagaggaaatttgggttgatgaagaaggcttatgagctgagcgtgctgtgtgactgtgagattgcgctgatcatcttcaacagcaccaacaagctgttccagtatgccagcaccgacatggacaaagtgcttctcaagtacacggagtacaacgagccgcatgagagccggacaaactcagacatcgtggagacgttgagaaagaagggccttaatggctgtgacagcccagaccccgatgcggacgattccgtaggtcacagccctgagtctgaggacaagtacaggaaaattaacgaagatattgatctaatgatcagcaggcaaagattgtgtgctgttccacctcccaacttcgagatgccagtctccatcccagtgtccagccacaacagtttggtgtacagcaaccctgtcagctcactgggaaaccccaacctattgccactggctcacccttctctgcagaggaatagtatgtctcctggtgtaacacatcgacctccaagtgcaggtaacacaggtggtctgatgggtggagacctcacgtctggtgcaggcaccagtgcagggaacgggtatggcaatccccgaaactcaccaggtctgctggtctcacctggtaacttgaacaagaatatgcaagcaaaatctcctcccccaatgaatttaggaatgaataaccgtaaaccagatctccgagttcttattccaccaggcagcaagaatacgatgccatcagtgtctgaggatgtcgacctgcttttgaatcaaaggataaataactcccagtcggctcagtcattggctaccccagtggtttccgtagcaactcctactttaccaggacaaggaatgggaggatatccatcagccatttcaacaacatatggtaccgagtactctctgagtagtgcagacctgtcatctctgtctgggtttaacaccgccagcgctcttcaccttggttcagtaactggctggcaacagcaacacctacataacatgccaccatctgccctcagtcagttgggagcttgcactagcactcatttatctcagagttcaaatctctccctgccttctactcaaagcctcaacatcaagtcagaacctgtttctcctcctagagaccgtaccaccaccccttcgagatacccacaacacacgcgccacgaggcggggagatctcctgttgacagcttgagcagctgtagcagttcgtacgacgggagcgaccgagaggatcaccggaacgaattccactcccccattggactcaccagaccttcgccggacgaaagggaaagtccctcagtcaagcgcatgcgactttctgaaggatgggcaacatgatcagattattacttactagtttttttttttttcttgcagtgtgtgtgtgtgctataccttaatggggaaggggggtcgatatgcattatatgtgccgtgtgtggaaaaaaaaaaagtcaggtactctgttttgtaaaagtacttttaaattgcctcagtgatacagtataaagataaacagaaatgctgagataagcttagcacttgagttgtacaacagaacacttgtacaaaatagattttaaggctaacttcttttcactgttgtgctcctttgcaaaatgtatgttacaatagatagtgtcatgttgcaggttcaacgttatttacatgtaaatagacaaaaggaaacatttgccaaaagcggcagatctttactgaaagagagagcagctgttatgcaacatatagaaaaatgtatagatgcttggacagacccggtaatgggtggccattggtaaatgttaggaacacaccaggtcacctgacatcccaagaatgctcacaaacctgcaggcatatcattggcgtatggcactcattaaaaaggatcagagaccattaaaagaggaccatacctattaaaaaaaaatgtggagttggagggctaacatatttaattaaataaataaataaatctgggtctgcatctcttattaaataaaaatataaaaatatgtacattacattttgcttattttcatataaaaggtaagacagagtttgcaaagcatttgtggctttttgtagtttacttaagccaaaatgtgtttttttccccttgatagcttcgctaatattttaaacagtcctgtaaaaaaccaaaaaggactttttgtatagaaagcactaccctaagccatgaagaactccatgctttgctaaccaagataactgttttctctttgtagaagttttgtttttgaaatgtgtatttctaattatataaaatattaagaatcttttaaaaaaatctgtgaaattaacatgcttgtgtatagctttctaatatatataatattatggtaatagcagaagttttgttatcttaatagcgggaggggggtatatttgtgcagttgcacatttgagtaactattttctttctgttttcttttactctgcttacattttataagtttaaggtcagctgtcaaaaggataacctgtggggttagaacatatcacattgcaacaccctaaattgtttttaatacattagcaatctattgggtcaactgacatccattgtatatactagtttctttcatgctatttttattttgttttttgcatttttatcaaatgcagggcccctttctgatctcaccatttcaccatgcatcttggaattcagtaagtgcatatcctaacttgcccatattctaaatcatctggttggttttcagcctagaatttgatacgctttttagaaatatgcccagaatagaaaagctatgttggggcacatgtcctgcaaatatggccctagaaacaagtgatatggaatttacttggtgaataagttataaattcccacagaagaaaaatgtgaaagactgggtgctagacaagaaggaagcaggtaaagggatagttgctttgtcatccgtttttaattattttaactgacccttgacaatcttgtcagcaatataggactgttgaacaatcccggtgtgtcaggacccccaaatgtcacttctgcataaagcatgtatgtcatctattttttcttcaataaagagatttaatagccatttcaagaaatcccataaagaacctctctatgtccctttttttaatttaaaaaaatgactcttgtctaatattcgtctataagggattaattttcagaccctttaataagtgagtgccataagaaagtcaatatatattgtttaaaagatatttcagtctaggaaagattttccttctcttggaatgtgaagatctgtcgattcatctccaatcatatgcattgacatacacagcaaagaagatataggcagtaatatcaacactgctatatcatgtgtaggacatttcttatccattttttctcttttacttgcatagttgctatgtgtttctcattgtaaaaggctgccgctgggtggcagaagccaagagaccttattaactaggctatatttttcttaacttgatctgaaatccacaattagaccacaatgcacctttggttgtatccataaaggatgctagcctgccttgtactaatgttttatatatt

SEQ ID NO: 53 - DNA, 1778 bp, Homo sapiens
tagaagtttacaatgaagtttcttctaatactgctcctgcaggccactgcttctggagctcttcccctgaacagctctacaagcctggaaaaaaataatgtgctatttggtgagagatacttagaaaaattttatggccttgagataaacaaacttccagtgacaaaaatgaaatatagtggaaacttaatgaaggaaaaaatccaagaaatgcagcacttcttgggtctgaaagtgaccgggcaactggacacatctaccctggagatgatgcacgcacctcgatgtggagtccccgatctccatcatttcagggaaatgccaggggggcccgtatggaggaaacattatatcacctacagaatcaataattacacacctgacatgaaccgtgaggatgttgactacgcaatccggaaagctttccaagtatggagtaatgttacccccttgaaattcagcaagattaacacaggcatggctgacattttggtggtttttgcccgtggagctcatggagacttccatgcttttgatggcaaaggtggaatcctagcccatgcttttggacctggatctggcattggaggggatgcacatttcgatgaggacgaattctggactacacattcaggaggcacaaacttgttcctcactgctgttcacgagattggccattccttaggtcttggccattctagtgatccaaaggctgtaatgttccccacctacaaatatgtcgacatcaacacatttcgcctctctgctgatgacatacgtggcattcagtccctgtatggagacccaaaagagaaccaacgcttgccaaatcctgacaattcagaaccagctctctgtgaccccaatttgagttttgatgctgtcactaccgtgggaaataagatctttttcttcaaagacaggttcttctggctgaaggtttctgagagaccaaagaccagtgttaatttaatttcttccttatggccaaccttgccatctggcattgaagctgcttatgaaattgaagccagaaatcaagtttttctttttaaagatgacaaatactggttaattagcaatttaagaccagagccaaattatcccaagagcatacattcttttggttttcctaactttgtgaaaaaaattgatgcagctgtttttaacccacgtttttataggacctacttctttgtagataaccagtattggaggtatgatgaaaggagacagatgatggaccctggttatcccaaactgattaccaagaacttccaaggaatcgggcctaaaattgatgcagtcttctattctaaaaacaaatactactatttcttccaaggatctaaccaatttgaatatgacttcctactccaacgtatcaccaaaacactgaaaagcaatagctggtttggttgttagaaatggtgtaattaatggtttttgttagttcacttcagcttaataagtatttattgcatatttgctatgtcctcagtgtaccactacttagagatatgtatcataaaaataaaatctgtaaaccataggtaatgattatataaaatacataatatttttcaattttgaaaactctaattgtccattcttgcttgactctactattaagtttgaaaatagttaccttcaaagcaagataattctatttgaagcatgctctgtaagttgcttcctaacatccttggactgagaaattatacttacttctggcataactaaaattaagtatatatattttggctcaaataaaattg

SEQ ID NO: 54 - DNA, 1668 bp, Homo sapiens
attctcagcacagcctttaaggttccaaacatctgctagaagaggaatgcagatttaaactgagtgaggtgtggagtgggggaagttgattgggtctagaccaaagaactttgaggaacttgcccagagccctgcatgcatcagacctacagcagacattgcaggcctgaagaaagcaccttttctgctgccatgacaaccatgcaaggaatggaacaggccatgccaggggctggccctggtgtgccccagctgggaaacatggctgtcatacattcacatctgtggaaaggattgcaagagaagttcttgaagggagaacccaaagtccttggggttgtgcagattctgactgccctgatgagccttagcatgggaataacaatgatgtgtatggcatctaatacttatggaagtaaccctatttccgtgtatatcgggtacacaatttgggggtcagtaatgtttattatttcaggatccttgtcaattgcagcaggaattagaactacaaaaggcctggtccgaggtagtctaggaatgaatatcaccagctctgtactggctgcatcagggatcttaatcaacacatttagcttggcgttttattcattccatcacccttactgtaactactatggcaactcaaataattgtcatgggactatgtccatcttaatgggtctggatggcatggtgctcctcttaagtgtgctggaattctgcattgctgtgtccctctctgcctttggatgtaaagtgctctgttgtacccctggtggggttgtgttaattctgccatcacattctcacatggcagaaacagcatctcccacaccacttaatgaggtttgaggccaccaaaagatcaacagacaaatgctccagaaatctatgctgactgtgacacaagagcctcacatgagaaattaccagtatccaacttcgatactgatagacttgttgatattattattatatgtaatccaattatgaactgtgtgtgtatagagagataataaattcaaaattatgttctcatttttttccctggaactcaataactcatttcactggctctttatcgagagtactagaagttaaattaataaataatgcatttaatgaggcaacagcacttgaaagtttttcattcatcataagaactttatataaaggcattacattggcaaataaggtttggaagcagaagagcaaaaaaaagatattgttaaaatgaggcctccatgcaaaacacatacttccctcccatttatttaactttttttttctcctacctatggggaccaaagtgctttttccttcaggaagtggagatgcatggccatctccccctccctttttccttctcctgcttttctttccccatagaaagtaccttgaagtagcacagtccgtccttgcatgtgcacgagctatcatttgagtaaaagtatacatggagtaaaaatcatattaagcatcagattcaacttatattttctatttcatcttcttcctttcccttctcccaccttctactgggcataattatatcttaatcatatatggaaatgtgcaacatatggtatttgttaaatacgtttgtttttattgcagagcaaaaataaatcaaattagaagcaataaaaaaaaaaaaaaaaaaaa

SEQ ID NO: 55 - DNA, 862 bp, Homo sapiens
gagaaccagagttaaaacctctttggagcttctgaggactcagctggaaccaacgggcacagttggcaacaccatcatgacatcacaacctgttcccaatgagaccatcatagtgctcccatcaaatgtcatcaacttctcccaagcagagaaacccgaacccaccaaccaggggcaggatagcctgaagaaacatctacacgcagaaatcaaagttattgggactatccagatcttgtgtggcatgatggtattgagcttggggatcattttggcatctgcttccttctctccaaattttacccaagtgacttctacactgttgaactctgcttacccattcataggaccctttttttttatcatctctggctctctatcaatcgccacagagaaaaggttaaccaagcttttggtgcatagcagcctggttggaagcattctgagtgctctgtctgccctggtgggtttcattatcctgtctgtcaaacaggccaccttaaatcctgcctcactgcagtggaactctctctctgatgctgatttgcactctgctggaattctgcctagctgtgctcactgctgtgctgcggtggaaacaggcttactctgacttccctgggagtggacttttcctgcctcacagttacattggtaattctggcatgtcctcaaaaatgactcatgactgtggatatgaagaactattgacttcttaagaaaaaagggagaaatattaatcagaaagttgattcttatgataatatggaaaagttaaccattatagaaaagcaaagcttgagtttcctaaatgtaagcttttaaagtaatgaacattaaaaaaaaccattatttcactgtc

SEQ ID NO: 56 - DNA, 4170 bp, Homo sapiens
gtagatgaggaaactgaagttgaggaatagtgaagagtttgtccaatgtcatagccccgtaatcaacgggacaaaaattttcttgctgatgggtcaagatggcatcgtgaagtggttgttcaccgtaaactgtaatacaatcctgtttatggatttgtttgcatatttttccccccatagggaaaccttttttccatggctcaggacacactcctggatcgagccaacaggagaactttctggtaagcatttggctaacttttttttttttgagatggagtcttgctgtgtcgcctaggctggagtgcagtggcgtgatcttggctcactgcagcctccacctcccgggttcaatcaattctcctacctcaacttcctgagtagctgggattacaggcgcccgccaccacacccggctcatttttgtacttttagtagagacacagttttgccatgttggccaggctggtcttgaattcctcagctcaggtgatatgcctgccttggcctctcaaagtgctgggattacaggcgtgagccactgtgcccggccttggctaacttttcaaaattaaagattttgacttgttacagtcatgtgacatttttttctttctgtttggtgagtttttgataatttatatctctcaaagtggagactttaaaaaagactcatctgtgtgccgtgttcactgcctggtatcttagtgtggaccgaagcctaaggaccctgaaaacagctgcagatgaagatggcaagcacccgctgcaagctggccaggtacctggaggacctggaggatgtggacttgaagaaatttaagatgcacttagaggactatcctccccagaagggctgcatccccctcccgaggggtcagacagagaaggcagaccatgtggatctagccacgctaatgatcgacttcaatggggaggagaaggcgtgggccatggccgtgtggatcttcgctgcgatcaacaggagagacctttatgagaaagcaaaaagagatgagccgaagtggggttcagataatgcacgtgtttcgaatcccactgtgatatgccaggaagacagcattgaagaggagtggatgggtttactggagtacctttcgagaatctctatttgtaaaatgaagaaagattaccgtaagaagtacagaaagtacgtgagaagcagattccagtgcattgaagacaggaatgcccgtctgggtgagagtgtgagcctcaacaaacgctacacacgactgcgtctcatcaaggagcaccggagccagcaggagagggagcaggagcttctggccatcggcaagaccaagacgtgtgagagccccgtgagtcccattaagatggagttgctgtttgaccccgatgatgagcattctgagcctgtgcacaccgtggtgttccagggggcggcagggattgggaaaacaatcctggccaggaagatgatgttggactgggcgtcggggacactctaccaagacaggtttgactatctgttctatatccactgtcgggaggtgagccttgtgacacagaggagcctgggggacctgatcatgagctgctgccccgacccaaacccacccatccacaagatcgtgagaaaaccctccagaatcctcttcctcatggacggcttcgatgagctgcaaggtgcctttgacgagcacataggaccgctctgcactgactggcagaaggccgagcggggagacattctcctgagcagcctcatcagaaagaagctgcttcccgaggcctctctgctcatcaccacgagacctgtggccctggagaaactgcagcacttgctggaccatcctcggcatgtggagatcctgggtttctccgaggccaaaaggaaagagtacttcttcaagtacttctctgatgaggcccaagccagggcagccttcagtctgattcaggagaacgaggtcctcttcaccatgtgcttcatccccctggtctgctggatcgtgtgcactggactgaaacagcagatggagagtggcaagagccttgcccagacatccaagaccaccaccgcggtgtacgtcttcttcctttccagtttgctgcagccccggggagggagccaggagcacggcctctgcgcccacctctgggggctctgctctttggctgcagatggaatctggaaccagaaaatcctgtttgaggagtccgacctcaggaatcatggactgcagaaggcggatgtgtctgctttcctgaggatgaacctgttccaaaaggaagtggactgcgagaagttctacagcttcatccacatgactttccaggagttctttgccgccatgtactacctgctggaagaggaaaaggaaggaaggacgaacgttccagggagtcgtttgaagcttcccagccgagacgtgacagtccttctggaaaactatggcaaattcgaaaaggggtatttgatttttgttgtacgtttcctctttggcctggtaaaccaggagaggacctcctacttggagaagaaattaagttgcaagatctctcagcaaatcaggctggagctgctgaaatggattgaagtgaaagccaaagctaaaaagctgcagatccagcccagccagctggaattgttctactgtttgtacgagatgcaggaggaggacttcgtgcaaagggccatggactatttccccaagattgagatcaatctctccaccagaatggaccacatggtttcttccttttgcattgagaactgtcatcgggtggagtcactgtccctggggtttctccataacatgcccaaggaggaagaggaggaggaaaaggaaggccgacaccttgatatggtgcagtgtgtcctcccaagctcctctcatgctgcctgttctcatggattggtgaacagccacctcacttccagtttttgccggggcctcttttcagttctgagcaccagccagagtctaactgaattggacctcagtgacaattctctgggggacccagggatgagagtgttgtgtgaaacgctccagcatcctggctgtaacattcggagattgtggttggggcgctgtggcctctcgcatgagtgctgcttcgacatctccttggtcctcagcagcaaccagaagctggtggagctggacctgagtgacaacgccctcggtgacttcggaatcagacttctgtgtgtgggactgaagcacctgttgtgcaatctgaagaagctctggttggtcagctgctgcctcacatcagcatgttgtcaggatcttgcatcagtattgagcaccagccattccctgaccagactctatgtgggggagaatgccttgggagactcaggagtcgcaattttatgtgaaaaagccaagaatccacagtgtaacctgcagaaactggggttggtgaattctggccttacgtcagtctgttgttcagctttgtcctcggtactcagcactaatcagaatctcacgcacctttacctgcgaggcaacactctcggagacaaggggatcaaactactctgtgagggactcttgcaccccgactgcaagcttcaggtgttggaattagacaactgcaacctcacgtcacactgctgctgggatctttccacacttctgacctccagccagagcctgcgaaagctgagcctgggcaacaatgacctgggcgacctgggggtcatgatgttctgtgaagtgctgaaacagcagagctgcctcctgcagaacctggggttgtctgaaatgtatttcaattatgagacaaaaagtgcgttagaaacacttcaagaagaaaagcctgagctgaccgtcgtctttgagccttcttggtaggagtggaaacggggctgccagacgccagtgttctccggtccctccagctgggggccctcaggtggagagagctgcgatccatccaggccaagaccacagctctgtgatccttccggtggagtgtcggagaagagagcttgccgacgatgccttcctgtgcagagcttgggcatctcctttacgccagggtgaggaagacaccaggacaatgacagcatcgggtgttgttgtcatcacagcgcctcagttagaggatgttcctcttggtgacctcatgtaattagctcattcaataaagcactttctttatttt

SEQ ID NO: 57 - DNA, 2038 bp, Homo sapiens
cggccacaacgagggagccgatttagatcctctgggcctgttccttccttttctttaaacgtcccagtctagcttagaggaggacctgttttgttagataaatggcaagcaaaaaacgagaagtccagttacagacagtcatcaataatcaaagcctgtgggatgagatgttgcagaacaaaggcttaacagtgattgatgtttaccaagcctggtgtggaccttgcagagcaatgcaacctttattcagaaaattgaaaaatgaactgaacgaagacgaaattctgcattttgctgtcgcagaagctgacaacattgtgactttgcagccatttagagataaatgtgaacctgtttttctctttagtgttaatggcaaaattatcgaaaagattcagggtgcaaatgcaccgcttgttaataaaaaagttattaatttgatcgatgaggagagaaaaattgcagcaggtgaaatggctcgacctcagtatcctgaaattccattagtagactcagattcagaagttagtgaagaatcaccatgtgaaagtgttcaggaattatacagtattgctattatcaaaccggatgctgtgattagtaaaaaagttctagaaattaaaagaaaaattaccaaagctggatttattatagaagcagagcataagacagtgctcactgaagaacaagttgtcaacttctatagtcgaatagcagaccagtgtgacttcgaagagtttgtctcttttatgacaagtggcttaagctatattctagttgtatctcaaggaagtaaacacaatcctccctctgaagaaaccgaaccacagactgacaccgaacctaacgaacgatctgaggatcaacctgaggtcgaagcccaggttacacctggaatgatgaagaacaaacaagacagtttacaagaatatctggaaagacaacatttagctcagctctgtgacattgaagaggatgcagctaatgttgctaagttcatggatgctttcttccccgattttaaaaaaatgaaaagcatgaaattagaaaagacattggcattacttcgaccaaatctctttcatgaaaggaaagatgatgttttgcgtattattaaagatgaagacttcaaaatactggagcaaagacaagtagtattatcggaaaaagaagcacaagcactgtgcaaggaatatgaaaatgaagactattttaataaacttatagaaaacatgaccagtggtccatctctagcccttgttttattgagagacaatggcttgcaatactggaaacaattactgggaccaagaactgttgaagaagccattgaatattttccagagagtttatgtgcacagtttgcgatggacagtttgccggtcaaccagttgtatggcagcgattcattagaaaccgctgaaagggaaatacagcatttctttcctcttcaaagcactttaggcttgattaaacctcatgcaacaagtgaacaaagagagcagatcctgaagatagttaaggaggctggatttgatctgacacaggtgaagaaaatgttcctaactcctgagcaaatagagaaaatttatccaaaagtaacaggaaaagacttttataaagatttattggaaatgttatctgtgggtccatctatggtcatgattctgaccaagtggaatgctgttgcagaatggagacgattgatgggcccaacagacccagaagaagcaaaattactttcccctgactccatccgagcccagtttggaataagtaaattgaaaaacattgtccatggagcatctaacgcctatgaagcaaaagaggttgttaatagactctttgaggatcctgaggaaaactaaagtatatactgtgaaaactttgagaagataatacatatgttcacgtcaatatacaaccatttggcacagcttcctgggaggaataataagaaaaacatgctttggaggaaaactcaagatacaaaaatgaatggctatgcataataacaataaaaatgtattccccaaac

SEQ ID NO: 58 - DNA, 3595 bp, Homo sapiens
aagccccacagtgaagaacatctgagctcaaatccagataagtgacataagtgacctgctttgtaaagccatagagatggcctgtccttggaaatttctgttcaagaccaaattccaccagtatgcaatgaatggggaaaaagacatcaacaacaatgtggagaaagccccctgtgccacctccagtccagtgacacaggatgaccttcagtatcacaacctcagcaagcagcagaatgagtccccgcagcccctcgtggagacgggaaagaagtctccagaatctctggtcaagctggatgcaaccccattgtcctccccacggcatgtgaggatcaaaaactggggcagcgggatgactttccaagacacacttcaccataaggccaaagggattttaacttgcaggtccaaatcttgcctggggtccattatgactcccaaaagtttgaccagaggacccagggacaagcctacccctccagatgagcttctacctcaagctatcgaatttgtcaaccaatattacggctccttcaaagaggcaaaaatagaggaacatctggccagggtggaagcggtaacaaaggagatagaaacaacaggaacctaccaactgacgggagatgagctcatcttcgccaccaagcaggcctggcgcaatgccccacgctgcattgggaggatccagtggtccaacctgcaggtcttcgatgcccgcagctgttccactgcccgggaaatgtttgaacacatctgcagacacgtgcgttactccaccaacaatggcaacatcaggtcggccatcaccgtgttcccccagcggagtgatggcaagcacgacttccgggtgtggaatgctcagctcatccgctatgctggctaccagatgccagatggcagcatcagaggggaccctgccaacgtggaattcactcagctgtgcatcgacctgggctggaagcccaagtacggccgcttcgatgtggtccccctggtcctgcaggccaatggccgtgaccctgagctcttcgaaatcccacctgaccttgtgcttgaggtggccatggaacatcccaaatacgagtggtttcgggaactggagctaaagtggtacgccctgcctgcagtggccaacatgctgcttgaggtgggcggcctggagttcccagggtgccccttcaatggctggtacatgggcacagagatcggagtccgggacttctgtgacgtccagcgctacaacatcctggaggaagtgggcaggagaatgggcctggaaacgcacaagctggcctcgctctggaaagaccaggctgtcgttgagatcaacattgctgtgctccatagtttccagaagcagaatgtgaccatcatggaccaccactcggctgcagaatccttcatgaagtacatgcagaatgaataccggtcccgtgggggctgcccggcagactggatttggctggtccctcccatgtctgggagcatcacccccgtgtttcaccaggagatgctgaactacgtcctgtcccctttctactactatcaggtagaggcctggaaaacccatgtctggcaggacgagaagcggagacccaagagaagagagattccattgaaagtcttggtcaaagctgtgctctttgcctgtatgctgatgcgcaagacaatggcgtcccgagtcagagtcaccatcctctttgcgacagagacaggaaaatcagaggcgctggcctgggacctgggggccttattcagctgtgccttcaaccccaaggttgtctgcatggataagtacaggctgagctgcctggaggaggaacggctgctgttggtggtgaccagtacgtttggcaatggagactgccctggcaatggagagaaactgaagaaatcgctcttcatgctgaaagagctcaacaacaaattcaggtacgctgtgtttggcctcggctccagcatgtaccctcggttctgcgcctttgctcatgacattgatcagaagctgtcccacctgggggcctctcagctcaccccgatgggagaaggggatgagctcagtgggcaggaggacgccttccgcagctgggccgtgcaaaccttcaaggcagcctgtgagacgtttgatgtccgaggcaaacagcacattcagatccccaagctctacacctccaatgtgacctgggacccgcaccactacaggctcgtgcaggactcacagcctttggacctcagcaaagccctcagcagcatgcatgccaagaacgtgttcaccatgaggctcaaatctcggcagaatctacaaagtccgacatccagccgtgccaccatcctggtggaactctcctgtgaggatggccaaggcctgaactacctgccgggggagcaccttggggtttgcccaggcaaccagccggccctggtccaaggcatcctggagcgagtggtggatggccccacaccccaccagacagtgcgcctggaggccctggatgagagtggcagctactgggtcagtgacaagaggctgcccccctgctcactcagccaggccctcacctacttcctggacatcaccacacccccaacccagctgctgctccaaaagctggcccaggtggccacagaagagcctgagagacagaggctggaggccctgtgccagccctcagagtacagcaagtggaagttcaccaacagccccacattcctggaggtgctagaggagttcccgtccctgcgggtgtctgctggcttcctgctttcccagctccccattctgaagcccaggttctactccatcagctcctcccgggatcacacgcccacggagatccacctgactgtggccgtggtcacctaccacacccgagatggccagggtcccctgcaccacggcgtctgcagcacatggctcaacagcctgaagccccaagacccagtgccctgctttgtgcggaatgccagcggcttccacctccccgaggatccctcccatccttgcatcctcatcgggcctggcacaggcatcgcgcccttccgcagtttctggcagcaacggctccatgactcccagcacaagggagtgcggggaggccgcatgaccttggtgtttgggtgccgccgcccagatgaggaccacatctaccaggaggagatgctggagatggcccagaagggggtgctgcatgcggtgcacacagcctattcccgcctgcctggcaagcccaaggtctatgttcaggacatcctgcggcagcagctggccagcgaggtgctccgtgtgctccacaaggagccaggccacctctatgtttgcggggatgtgcgcatggcccgggacgtggcccacaccctgaagcagctggtggctgccaagctgaaattgaatgaggagcaggtcgaggactatttctttcagctcaagagccagaagcgctatcacgaagatatctttggtgctgtatttccttacgaggcgaagaaggacagggtggcggtgcagcccagcagcctggagatgtcagcgctctgagggcctacaggaggggttaaagctgccggcacagaacttaaggatggagccagctct

SEQ ID NO: 59 - DNA, 2343 bp, Homo sapiens
  misc_feature (2239)..(2239): n is a, c, g, or t
  misc_feature (2284)..(2284): n is a, c, g, or t
  misc_feature (2322)..(2322): n is a, c, g, or t
  misc_feature (2339)..(2339): n is a, c, g, or t
gcgcggccccgaaccgccgccaggccggcacgggggaaggagccggtgggggtagggggtgcggtggggggtggggaccctccggctcttgggggtcccagtccccgccggctgctgagcgggtggggtggtggaggagctgcagagatgtccggccagagcctgacggaccgaatcactgccgcccagcacagtgtcaccggctctgccgtatccaagacagtatgcaaggccacgacccacgagatcatggggcccaagaaaaagcacctggactacttaattcagtgcacaaatgagatgaatgtgaacatcccacagttggcagacagtttatttgaaagaactactaatagtagttgggtggtggtcttcaaatctctcattacaactcatcatttgatggtgtatggaaatgagcgttttattcagtatttggcttcaagaaacacgttgtttaacttaagcaattttttggataaaagtggattgcaaggatatgacatgtctacatttattaggcggtatagtagatatttaaatgagaaagcagtttcatacagacaagttgcatttgatttcacaaaagtgaagagaggggctgatggagttatgagaacaatgaacacagaaaaactcctaaaaactgtaccaattattcagaatcaaatggatgcacttcttgattttaatgttaatagcaatgaacttacaaatggggtaataaatgctgccttcatgctcctgttcaaagatgccattagactgtttgcagcataccatgaaggaattattaatttgttggaaaaatattttgatatgaaaaagaaccaatgcaaagaaggtcttgacatctataagaagttcctaactaggatgacaagaatctcagagttcctcaaagttgcagagcaagttggaattgacagaggtgatataccagacctttcacaggcccctagcagtcttcttgatgctttggaacaacatttagcttccttggaaggaaagaaaatcaaagattctacagctgcaagcagggcaactacactttccaatgcagtgtcttccctggcaagcactggtctatctctgaccaaagtggatgaaagggaaaagcaggcagcattagaggaagaacaggcacgtttgaaagctttaaaggaacagcgcctaaaagaacttgcaaagaaacctcatacctctttaacaactgcagcctctcctgtatccacctcagcaggagggataatgactgcaccagccattgacatattttctacccctagttcttctaacagcacatcaaagctgcccaatgatctgcttgatttgcagcagccaacttttcacccatctgtacatcctatgtcaactgcttctcaggtagcaagtacatggggagatcctttctctgctactgtagatgctgttgatgatgccattccaagcttaaatcctttcctcacaaaaagtagtggtgatgttcacctttccatttcttcagatgtatctacttttactactaggacacctactcatgaaatgtttgttggattcactccttctccagttgcacagccacacccttcagctggccttaatgttgactttgaatctgtgtttggaaataaatctacaaatgttattgtagattctgggggctttgatgaactaggtggacttctcaaaccaacagtggcctctcagaaccagaaccttcctgttgccaaactcccacctagcaagttagtatctgatgacttggattcatctttagccaaccttgtgggcaatcttggcatcggaaatggaaccactaagaatgatgtaaattggagtcaaccaggtgaaaagaagttaactgggggatctaactgcgaaccaaaggttgcaccaacaaccgcttggaatgctgcaacaatggcaccccctgtaatggcctatcctgctactacaccaacaggcatgataggatatggaattcctccacaaatgggaagtgttcctgtaatgacgcaaccaaccttaatatacagccagcctgtcatgagacctccaaacccctttggccctgtatcaggagcacagatacagtttatgtaacttgatggaagaaaatggaattactccaaaaagacaagtgctcaagcagcaaaatccttacttccagcaaaatccaaactgctgtctcttaaatctcttaaactctcttcttccattaggatgctacaagtanctcagtgaaggcccatgaagggaattggggactagtttatagggngaacgtattcattacagtttataaaggccaggattggnttggattttaggattangttc

SEQ ID NO: 60 - DNA, 1323 bp, Homo sapiens
aataggggaaaataagccagatggataaaggaagtgctggtcaccctggaggtgcactggtttggggaaggctcctggcccccacagccctcttcggagcctgagcccggctctcctcactcacctcaacccccaggcggcccctccacagggcccctctcctgcctggacggctctgctggtctccccgtcccctggagaagaacaaggccatgggtcggcccctgctgctgcccctactgcccttgctgctgccgccagcatttctgcagcctagtggctccacaggatctggtccaagctacctttatggggtcactcaaccaaaacacctctcagcctccatgggtggctctgtggaaatccccttctccttctattacccctgggagttagccacagctcccgacgtgagaatatcctggagacggggccacttccacaggcagtccttctacagcacaaggccgccttccattcacaaggattatgtgaaccggctctttctgaactggacagagggtcagaagagcggcttcctcaggatctccaacctgcagaagcaggaccagtctgtgtatttctgccgagttgagctggacacacggagctcagggaggcagcagtggcagtccatcgaggggaccaaactctccatcacccaggctgtcacgaccaccacccagaggcccagcagcatgactaccacctggaggctcagtagcacaaccaccacaaccggcctcagggtcacacagggcaaacgacgctcagactcttggcacataagtctggagactgctgtgggggtggcagtggctgtcactgtgctcggaatcatgattttgggactgatctgcctcctcaggtggaggagaaggaaaggtcagcagcggactaaagccacaaccccagccagggaacccttccaaaacacagaggagccatatgagaatatcaggaatgaaggacaaaatacagatcccaagctaaatcccaaggatgacggcatcgtctatgcttcccttgccctctccagctccacctcacccagagcacctcccagccaccgtcccctcaagagcccccagaacgagaccctgtactctgtcttaaaggcctaaccaatggacagccctctcaagactgaatggtgaggccaggtacagtggcgcacacctgtaatcccagctactctgaagcctgaggcagaatcaagtgagcccaggagttcagggccagctttgataatggagcgagatgccatctctagttaaaaatatatattaacaataaagtaacaaatttaaaaagataaaaaaa

SEQ ID NO: 61 - DNA, 4242 bp, Homo sapiens
gaattcggcgctgagtgacccgagtcgggacgcgggctgcgcgcgcgggaccccggagcccaaacccggggcaggcgggcagctgtgcccgggcggcacggccagcttcctgatttctcccgattccttccttctccctggagcggccgacaatgtccaccacggtcaatgtagattcccttgcggaatatgagaagagccagatcaagagagccctggagctggggacggtgatgactgtgttcagcttccgcaagtccacccccgagcggagaaccgtccaggtgatcatggagacgcggcaggtggcctggagcaagaccgccgacaagatcgagggcttcttggatatcatggaaataaaagaaatccgcccagggaagaactccaaagatttcgagcgagcaaaagcagttcgccagaaagaagactgctgcttcaccatcctatatggcactcagttcgtcctcagcacgctcagcttggcagctgactctaaagaggatgcagttaactggctctctggcttgaaaatcttacaccaggaagcgatgaatgcgtccacgcccaccattatcgagagttggctgagaaagcagatatattctgtggatcaaaccagaagaaacagcatcagtctccgagagttgaagaccatcttgcccctgatcaactttaaagtgagcagtgccaagttccttaaagataagtttgtggaaataggagcacacaaagatgagctcagctttgaacagttccatctcttctataaaaaacttatgtttgaacagcaaaaatcgattctcgatgaattcaaaaaggattcgtccgtgttcatcctggggaacactgacaggccggatgcctctgctgtttacctgcatgacttccagaggtttctcatacatgaacagcaggagcattgggctcaggatctgaacaaagtccgtgagcggatgacaaagttcattgatgacaccatgcgtgaaactgctgagcctttcttgtttgtggatgagttcctcacgtacctgttttcacgagaaaacagcatctgggatgagaagtatgacgcggtggacatgcaggacatgaacaaccccctgtctcattactggatctcctcgtcacataacacgtaccttacaggtgaccagctgcggagcgagtcgtccccagaagcttacatccgctgcctgcgcatgggctgtcgctgcattgaactggactgctgggacgggcccgatgggaagccggtcatctaccatggctggacgcggactaccaagatcaagtttgatgacgtcgtgcaggccatcaaagaccacgcctttgttacctcgagcttcccagtgatcctgtccatcgaggagcactgcagcgtggagcaacagcgtcacatggccaaggccttcaaggaagtatttggcgacctgctgttgacgaagcccacggaggccagtgctgaccagctgccctcgcccagccagctgcgggagaagatcatcatcaagcataagaagctgggcccccgaggcgatgtggatgtcaacatggaggacaagaaggacgaacacaagcaacagggggagctgtacatgtgggattccattgaccagaaatggactcggcactactgcgccattgctgatgccaagctgtccttcagtgatgacattgaacagactatggaggaggaagtgccccaggatataccccctacagaactacattttggggagaaatggttccacaagaaggtggagaagaggacgagtgccgagaagttgctgcaggaatactgcatggagacggggggcaaggatggcaccttcctggttcgggagagcgagaccttccccaatgactacaccctgtccttctggcggtcaggccgggtccagcactgccggatccgctccaccatggagggcgggaccctgaaatactacttgactgacaacctgaggttcaggaggatgtatgccctcatccagcactaccgcgagacgcacctgccgtgcgccgagttcgagctgcggctcacggaccctgtgcccaaccccaacccccacgagtccaagccgtggtactatgacagcctgagccgcggagaggcagaggacatgctgatgaggattccccgggacggggccttcctgatccggaagcgagaggggagcgactcctatgccatcaccttcagggctaggggcaaggtaaagcattgtcgcatcaaccgggacggccggcactttgtgctggggacctccgcctattttgagagtctggtggagctcgtcagttactacgagaagcattcactctaccgaaagatgagactgcgctaccccgtgacccccgagctcctggagcgctacaatacggaaagagatataaactccctctacgacgtcagcagaatgtatgtggatcccagtgaaatcaatccgtccatgcctcagagaaccgtgaaagctctgtatgactacaaagccaagcgaagcgatgagctgagcttctgccgtggtgccctcatccacaatgtctccaaggagcccgggggctggtggaaaggagactatggaaccaggatccagcagtacttcccatccaactacgtcgaggacatctcaactgcagacttcgaggagctagaaaagcagattattgaagacaatcccttagggtctctttgcagaggaatattggacctcaatacctataacgtcgtgaaagcccctcagggaaaaaaccagaagtcctttgtcttcatcctggagcccaaggagcagggcgatcctccggtggagtttgccacagacagggtggaggagctctttgagtggtttcagagcatccgagagatcacgtggaagattgacagcaaggagaacaacatgaagtactgggagaagaaccagtccatcgccatcgagctctctgacctggttgtctactgcaaaccaaccagcaaaaccaaggacaacttagaaaatcctgacttccgagaaatccgctcctttgtggagacgaaggctgacagcatcatcagacagaagcccgtcgacctcctgaagtacaatcaaaagggcctgacccgcgtctacccaaagggacaaagagttgactcttcaaactacgaccccttccgcctctggctgtgcggttctcagatggtggcactcaatttccagacggcagataagtacatgcagatgaatcacgcattgttttctctcaacgggcgcacgggctacgttctgcagcctgagagcatgaggacagagaaatatgacccgatgccacccgagtcccagaggaagatcctgatgacgctgacagtcaaggttctcggtgctcgccatctccccaaacttggacgaagtattgcctgtccctttgtagaagtggagatctgtggagccgagtatggcaacaacaagttcaagacgacggttgtgaatgataatggcctcagccctatctgggctccaacacaggagaaggtgacatttgaaatttatgacccaaacctggcatttctgcgctttgtggtttatgaagaagatatgttcagcgatcccaactttcttgctcatgccacttaccccattaaagcagtcaaatcaggattcaggtccgttcctctgaagaatgggtacagcgaggacatagagctggcttccctcctggttttctgtgagatgcggccagtcctggagagcgaagaggaactttactcctcctgtcgccagctgaggaggcggcaagaagaactgaacaaccagctctttctgtatgacacacaccagaacttgcgcaatgccaaccgggatgccctggttaaagagttcagtgttaatgagaaccactccagctgtaccaggagaaatgcaacaagaggttaagagagaagagagtcagcaacagcaagttttactcatagaagctggggtatgtgtgtaagggtattgtgtgtgtgcgcatgtgtgtttgcatgtaggagaacgtgccctattcacactctgggaagacgctaatctgtgacatcttttcttcaagcctgccatcaaggacatttcttaagacccaactggcatgagttggggtaatttcctattattttcatcttggacaacttctaacttatatctttatagaggattccccaaaatgtgctcctcatttttggcctctcatgttccaaacctcattgaataaaaagcaatgaaaaccttg

SEQ ID NO: 62 - DNA, 3416 bp, Homo sapiens
cggtacaggtaagtcggccgggcaggtaggggtgcccgaggagtagtcgctggagtccgcgcctccctgggactgcaatgtgccggtcttagctgctgcctgagaggatgtctggggtgtccgagcccctgagccgagtaaagttgggcacattacgccggcctgaaggccctgcagagcccatggtggtggtaccagtagatgtggaaaaggaggacgtgcgtatcctcaaggtctgcttctatagcaacagcttcaatcctgggaagaacttcaaactggtcaaatgcactgtccagacggagatccgggagatcatcacctccatcctgctgagcgggcggatcgggcccaacatccggttggctgagtgctatgggctgaggctgaagcacatgaagtccgatgagatccactggctgcacccacagatgacggtgggtgaggtgcaggacaagtatgagtgtctgcacgtggaagccgagtggaggtatgaccttcaaatccgctacttgccagaagacttcatggagagcctgaaggaggacaggaccacgctgctctatttttaccaacagctccggaacgactacatgcagcgctacgccagcaaggtcagcgagggcatggccctgcagctgggctgcctggagctcaggcggttcttcaaggatatgccccacaatgcacttgacaagaagtccaacttcgagctcctagaaaaggaagtggggctggacttgtttttcccaaagcagatgcaggagaacttaaagcccaaacagttccggaagatgatccagcagaccttccagcagtacgcctcgctcagggaggaggagtgcgtcatgaagttcttcaacactctcgccggcttcgccaacatcgaccaggagacctaccgctgtgaactcattcaaggatggaacattactgtggacctggtcattggccctaaagggatccgccagctgactagtcaggacgcaaagcccacctgcctggccgagttcaagcagatcaggtccatcaggtgcctcccgctggaggagggccaggcagtacttcagctgggcattgaaggtgccccccaggccttgtccatcaaaacctcatccctagcagaggctgagaacatggctgacctcatagacggctactgccggctgcagggtgagcaccaaggctctctcatcatccatcctaggaaagatggtgagaagcggaacagcctgccccagatccccatgctaaacctggaggcccggcggtcccacctctcagagagctgcagcatagagtcagacatctacgcagagattcccgacgaaaccctgcgaaggcccggaggtccacagtatggcattgcccgtgaagatgtggtcctgaatcgtattcttggggaaggcttttttggggaggtctatgaaggtgtctacacaaatcacaaaggggagaaaatcaatgtagctgtcaagacctgcaagaaagactgcactctggacaacaaggagaagttcatgagcgaggcagtgatcatgaagaacctcgaccacccgcacatcgtgaagctgatcggcatcattgaagaggagcccacctggatcatcatggaattgtatccctatggggagctgggccactacctggagcggaacaagaactccctgaaggtgctcaccctcgtgctgtactcactgcagatatgcaaagccatggcctacctggagagcatcaactgcgtgcacagggacattgctgtccggaacatcctggtggcctcccctgagtgtgtgaagctgggggactttggtctttcccggtacattgaggacgaggactattacaaagcctctgtgactcgtctccccatcaaatggatgtccccagagtccattaacttccgacgcttcacgacagccagtgacgtctggatgttcgccgtgtgcatgtgggagatcctgagctttgggaagcagcccttcttctggctggagaacaaggatgtcatcggggtgctggagaaaggagaccggctgcccaagcctgatctctgtccaccggtcctttataccctcatgacccgctgctgggactacgaccccagtgaccggccccgcttcaccgagctggtgtgcagcctcagtgacgtttatcagatggagaaggacattgccatggagcaagagaggaatgctcgctaccgaacccccaaaatcttggagcccacagccttccaggaacccccacccaagcccagccgacctaagtacagaccccctccgcaaaccaacctcctggctccaaagctgcagttccaggttcctgagggtctgtgtgccagctctcctacgctcaccagccctatggagtatccatctcccgttaactcactgcacaccccacctctccaccggcacaatgtcttcaaacgccacagcatgcgggaggaggacttcatccaacccagcagccgagaagaggcccagcagctgtgggaggctgaaaaggtcaaaatgcggcaaatcctggacaaacagcagaagcagatggtggaggactaccagtggctcaggcaggaggagaagtccctggaccccatggtttatatgaatgataagtccccattgacgccagagaaggaggtcggctacctggagttcacagggcccccacagaagcccccgaggctgggcgcacagtccatccagcccacagctaacctggaccggaccgatgacctggtgtacctcaatgtcatggagctggtgcgggccgtgctggagctcaagaatgagctctgtcagctgccccccgagggctacgtggtggtggtgaagaatgtggggctgaccctgcggaagctcatcgggagcgtggatgatctcctgccttccttgccgtcatcttcacggacagagatcgagggcacccagaaactgctcaacaaagacctggcagagctcatcaacaagatgcggctggcgcagcagaacgccgtgacctccctgagtgaggagtgcaagaggcagatgctgacggcttcacacaccctggctgtggacgccaagaacctgctcgacgctgtggaccaggccaaggttctggccaatctggcccacccacctgcagagtgacggagggtgggggccacctgcctgcgtcttccgcccctgcctgccatgtacctcccctgccttgctgttggtcatgtgggtcttccagggagaaggccaaggggagtcaccttcccttgccactttgcacgacgccctctccccacccctacccctggctgtactgctcaggctgcagctggacagaggggactctgggctatggacacagggtgacggtgacaaagatggctcagagggggactgctgctgcctggccactgctccctaagccagcct

SEQ ID NO: 63 - DNA, 2424 bp, Homo sapiens
actgtctctagcagtgggtgaaggcctgtgagtgaggaatgcctctcaccagctgtgcctgagctgcagcactccagccactgctgtctccttagctgctcacatatggatactttcacagttcaggattccactgcaatgagctggtggaggaataatttctggatcatcttagctgtggccatcatcgttgtctctgtgggtctgggcctcatcctgtactgtgtctgtaagtggcagcttagacgaggcaagaaatgggaaattgccaagcccctgaaacacaagcaagtagatgaagaaaagatgtatgagaatgttcttaatgagtcgccagttcaattaccgcctctgccaccgaggaattggccttctctagaagactcttccccacaggaagccccaagtcagccgcccgctacatactcactggtaaataaagttaaaaataagaagactgtttccatcccaagctacattgagcctgaagatgactatgacgatgttgaaatccctgcaaatactgaaaaagcatcattttgaaacagccatttcttctttttggcaaaactgaagagggttcacacaacttattttaaaacaatcaagaatggttgaacttcagtaggtctctgggccctgaaagccagtggtgattttatgaagctctataagataaagcacttcccaaaccttagatgaagacacccctgcgatcggatgactgcagccagaggagacacatgggtgctcggctctgaggacttagaggggtcagccttgtgctgttgaggaaactttccatgggaaggaccacggggctccatggctcccacctgtgggaaactactcatttcttggcattctttcccccttcattccctttggtttgcatggttctgagtgatattaaatctcagcatttggttgtgcagaccctcccaggctcccatccccagcaaggccctcaccaagcatgctggtctttaccctctcaccccacccacctcctgcactgtgaggctgtgggtgagttacagctgagtgctctcgtgcccaggttcccacaccacatctcgcgagtttgcaagggcagggagtaccttttgttctcgtgaaccctccccacctagacaccctgcaaaccccagtgcctttatatgatgtaggccaaattgaccatagagatttgagttttcacctaggttttctccccgtgcttgcaagttgtactgtaacaatggacaaaggacaaaagttaccttctgatttacacctagaagcatcattttgcaataggtgtgttgggggtgctacaggaaaaatacatttcccccaggacaaatcatggggaacaggaaagaaaaggggcatgtaacaatggcatatacaagatgagagttcagggggcttaatatcccctgtccatcattttcatcagtacttactcgagttctaggaaaacagcctcaagccccttccttccagatcactgtccctgggcatctgggaggaggcagaaggtccactgtgatgtgctgcagccaatgagatgggccagggacatgggcagatgtcttgttaaacaagtgtcctaatggggtcaacaaggcccgagtcagctttataggctcttagacctcatcaattccttctagctgatcgccagagccctaggacttgactcattctaactatactcacaagatgctggtttctaagtgacctctgggaaatctggcaaatgaacagccttgcagagagagcactgtgaacctggaaaggcctgagagtgactcagatttccctcaagagatgggaaaatgtgttcctcccattttcaagctttctccctcaatcaacgctggagcactggggacctgggcttcctccctggttctctctttccagactctatgaaggcttccaccttgctattaatacctccttgggaggccaaggtgggcggatcacctgaggtcgggagttcgagaccagcctgaccaacatggagaaaccccatttctactaaaaatacaaaattagtcaggcatggtcgcgcatgcctgtaatcccagctacttgggaggctgaggcagaagaatcgcttgaaactgggaggcggaggttgcggtgagccgagaacatgccattgcactccagcctggacaacaagagtgaaactccatctaaaaataaataaataaataaataaataaaccctccttatgttaggccagtagttatctaactatggccttatgggactctggtatcccaccagccaaagagaggactcttcccaaattatagaacaaaaataagccaaaggattggagtgtttcaaacacatgctttcgtcttataaatgttctgtaaaccctccatgactatgacaaaagttaaaaacaaatgccagacaaa

SEQ ID NO: 64 - DNA, 9757 bp, Homo sapiens
agacggcacccaggcgctccgggatggcgctccgcgggaccctccggccgctcaaagttcgcaggaggcgagagatgctgccgcagcaagtcggcttcgtgtgcgcggtgctggccctggtgtgctgtgcgtccggcctcttcggcagcttggggcacaaaacagcttctgctagcaaacgtgtcctgccagacacgtggagaaatagaaagttgatggccccagtgaatgggacacagacagccaagaactgcacagatcctgcgattcacgagttccccacagatctgttctccaataaggagcgacagcacggagccgtcctgctgcacatccttggtgctctgtatatgttctatgccttggccatagtgtgcgatgacttctttgttccgtctctagagaagatctgtgagagactccatctgagcgaagatgtggctggagccaccttcatggctgcaggaagctcaacgccagagctgtttgcgtctgttattggggtgttcatcacccatggggacgtcggggtgggcaccatcgtgggctctgctgtgttcaacatcctgtgcataattggagtgtgcggactgtttgctggccaggtggtccgtctgacgtggtgggccgtgtgccgagactccgtgtactacaccatctctgtcatcgtgctcatcgtgttcatatatgatgaacaaattgtgtggtgggaaggcctggtgctcatcatcttgtatgtgttttatattctgatcatgaagtacaatgtgaagatgcaagcctttttcacagtcaaacaaaagagcattgcaaacggtaacccggtcaacagtgagctggaggctggtaatgatttctatgacggtagctatgatgacccttccgtgccattgctggggcaagtgaaggagaagccacagtatggcaagaaccccgtggtgatggtggacgagattatgagctccagccctcccaagttcaccttccctgaagcaggcttacgaatcatgatcaccaataagtttggacccaggacccgactacggatggccagcaggatcatcattaatgagcggcagagactgatcaactcggccaatggtgtgagcagtaagccgcttcaaaacgggaggcacgagaacattgagaacgggaatgttcctgtggaaaaccccgaagaccctcagcagaatcaggagcagcagccgccgccacagccaccaccgccagagccagagccggtggaggctgacttcctgtcccccttctccgtgccggaggccagaggggacaaggtcaagtgggtgttcacctggcccctcatcttcctcctgtgcgtcaccattcccaactgcagcaagccccgctgggagaagttcttcatggtcaccttcatcaccgccacgctgtggatcgctgtgttctcctacatcatggtgtggctggtgactattatcggatacacacttgggatcccggatgtcatcatgggcattactttcctggcagcagggacaagtgttccagactgcatggccagcctaattgtggcgagacaaggccttggggacatggcagtctccaacaccataggaagcaacgtgtttgacatcctggtaggacttggtgtaccgtggggcctgcagaccatggttgttaattatggatcaacagtgaagatcaacagccgggggctggtctattccgtggtcctgttgctgggctctgtcgctctcaccgtcctcggcatccacctaaacaagtggcgactggaccggaagctgggtgtctacgtgctggttctctacgccatcttcttgtgcttctccataatgatagagtttaacgtctttaccttcgtcaacttgccgatgtgccgggaagacgattagcgctgagtcgcggcccctgggagctgatctggacaccctgtgacactggcgttctcctctcccctccttcccccaccacaggtctctcctgcataggcagccactgtccgttctttcacacactggaaggaagagccatcgtggtctttgtctggccacaggccaggctgctgggcatcctcctcctccttggagttccgcccctgcaaggctggatttgggggccattatctgagcagcttcaaagacccctgagctgccaaccacggagatgtgccaagcatctcatctctcctgcacactttagtcagaaggacttctgcatgcagtttgtctttctgttctgcaggcagcttcagaattgaggtcatttgtgagcacaagatctcatagggcaggtgcaaaataggaatgttgttctcaagtgtcacctccagcccagaggtggttccttaggcagcatgtgctcctgggagcctctgacttttgctggaagcagccacagtttggaaggggcaagacctcaacctgttggggtttagggcccatgatggcagacattctaccccttttcctggaaaaactggaagaatgaaaataatttttttctgtggaagagagaaaatgagtgaatattcttctcacttttattgatgcattcagagaataagcaatgaaatattaaaaaatgaaacatcatataggtcatcatacttgaaaattatcattccatatgaaaggatcatgatacacaccaaaaaagtaatgatcgtaaagacacaaatcctctgtatgccatcttgcattggcactgaggtgtttggtttggaatagggaaaaaggtaagagactaacgtggaaaggtgctaactcagagactggagattatagtttacagctgtactttccagatcttctatgtgacacaatgcactgtccttgtgggtttgtcatttattggttaatgctctagtttcaaaaccaccctgttgaaagttccagttatttatatgcccaacaaatttcatagcctgctgaactgaactgagtgtgtcagaagtgctggttaatgacgagaagagattgcctgaaaaacaacaaactgctttctggttagctgaaggcaagtgtgaaaatcagaatttagaatatttagagctaagcttctggaaccacgtagtttctacacgtggcaggccaagaatgggaggctgactcaaaactagatagaaaaatataaaataatcttcgaccacttgatagctctcaaatatatatttaaaagatttatgaatacaaaccatttatggtttatgatttctaaaaagaaagcacaattaattttatagagaggttttttatttttttaatatttctattgcaaaagtctatccgatttgatgcactttgaatattgagatattttgcacggatgaatgtatgggaactacccatgatgatgtaagaggaaagaacatttttttgtgattcaccagacatcactttaaacttggtgatgagtttaaatccagtagctaatcccttcctgagactcaaagatcgtgacgctggttggaatttctgactgtgccctttagggcctcctgagtttcaaaaggaggaagtgttcgtgcttgtgtccctgaagttccctgttgcatgagcctgcgacaggacctcacccccaccaccaggcttctatttgggattcacatcagtattagtatcgtagctacaccaagttcaggcttctctttttgtttttttacctagaaattgggctcagtggtcttcaacttgaggacgagggtgattttcctaagaaatcagcaaagagggaaggcagggcccctgtagattcaccagtataaacttcagctgcagggattccagagccctcgggaccactctgtcaccttaatagccaagttctcctggttcctccgatcttacaggctcatccaggttccaaagtgcttctgtctctgttttgattctccaaactgctctgtgatgtatgtagggattattctccccacttaacagaaagtagtgtcttggagaggtcaagggtctctagttcaatggccagtcatagcagaagggaggccaagcaccagtccatcacccctcccaggccagcctctgtaagttggccacacttggggagtgagtgtgggtatgactttaccctcctggttggttcttactgtttgagtcaaaacctcatcaatatatcattgactcctgggttcctcaggtcatttcctaatatctgtccctatccaatgcctctattttatcttgaaaaaaggaccaaaaattatttttagctatggcaaggcacaggccacatggcccctgatggcgtccctgctggttttcaattctctgaagccttgtgtagctttcagagcacacgtatcctaattaccctcctcttcctcagcagaacccatttgagattctaaatgaatactcttagtctctaaagttgcagttagaaactaaaataatgttttttaatatgtaatatgctcctcttggctaattttcttttgactttaatgtgccaatgtaacttcctttaaaggatctatgcatttattaaatctggaaaactatatgtacactgtaggtggaaaattctcttttttaactaaatatttttccatcacaaatttaaagaattgcatgattaattaggctttcatttttaaattacgctttcatcactacgcaggattactttattttattcccaaagctcattagcatgggataattactctgctacagaaataggcaatttaaaaaaatgaatttagctcttctcattgggggcagaaaagaaaaaaaaaaccattgcactcagatggaaaatgcctatagacacaggagcaggtggttcctgtggacttctggtttggaattttgcctcaccaggtcaagcgtggttagggtggaaggtgtccagtatcttgaaaacctggccctggaggaaggttctgggtcagctgcaatgagagactggtgattaagggcaccgtgggcaggacacagtcctcgccttacccaccccatccttcctgttacccacagtctgctggcctccatgcctcttccccttgtcacttgtgtctcctccttatgcacagagctgcctgcctttatgaattttcttttcttttttttttgagacagcgtcttgctgtgtcacccaggctggagtgcagtggtgccatcttggctcactgcaacctccgcctcccaggttcaagcaattcttgtgcctcagcctcctgaggattacaggcgtgcgccaccacacccagctaatttttgtatttttagtagagacgggttttcaccatgttggccaggctggtctcaaactcctgacctcaggtgatccacccacctcggtttcccaaagtgctgggattacaggtgtgagccaccacgcccagcctgcccttgtgaattttcacctgctccttacccctcacctgttaggactgtttcttgcttttgcccctgtcggtcccctgccttaacagacctaagcagctgataatgcaccaagcttccctgaccaggtggggtgtgtctatcacccaagggcagtcctacagaccctgaccaaaggccgttcctgggcggcccaaggtccaggtttcttccacctgctcttccctgtttatggggatttgcaagcctaattgcatcagcaggagcccatctctcagagaacccggactccccaagcagactgggattttgggaagggtgtggggggtgtcattgctggatacccgtctttctgcctgtcctttctcctctctgaatcctggggcccctctccctccttaaagctggagtggacagagggacaggagaggatcagagttcatcccccctgggaaagagcaagagcgaatgaatcccagcgccagcggctgaggctgccttccgtgccttccctccatgggcgacgggtgagtggggcttaggaaactggaacagggaaggttctgttaccacactttggaactttccccctgggattcagcagttgagaagcagagacctttctgccctgggtgaatgggtccttgggggaggggttggtcttttgtctcgcatccccatctttcctttccttctgggccatgctcctccctggctggaaaaaggtggctgtgctgtccctgtgatccactctcagcaaatgcgtgtggctcaaataaacaaagaacttacctgttagagtgaaaatcctcaggagattgtacccaaatgccatgctctaaatattcatggtctctctaatgccctcaagacgtgatttccatgggaaccatcctcccctgggggcagttagcaggagtacgtggggcacgtgaggtggtcctcctttcagcacaccgtgcccatagaaacttctagaaatttctgaaaatgctctgtgggcagctcttgggtggcagtaagtccatcaacccccatctaccccgggcctgaagcgctgcgcttgctctctttatgtgtgtgcacccgaaggatttcctggtctctgtagctgatcctgtgagcccctcaagcatgaagcctcccttggggcttctcaaagcatggagaggggcccttcctgtcctttgggaaaatcttccccactgtgtcagttatatgggaacaagagtgatggggtctttctctaggcctgtgccacaggacagagaacacgggattctgctgttcgctttgagccacagcctttaccagcccggcttgtgtggggggccccttcgccttgctgcaaagagctgttccccaaagggcatatccacagggtacaggttttaaaaaggcttttttttttttttttgagacagggtctcgctctgtcgcctagactcagtgcagtggcgccatgttggctggttgcaacctccacctcctgggttcaagtgattctcccacctcagcctctctggtagctgggactacaggcacgcgccaccatgcccagctaatttttggatttttagtagagaaggagtttcaccatgctgaccaggctggtttcgaactcctgacctcaagtgatccgcccgcctgggcctcccagagtgctgagattacaggcgtgagccaccgcacctggccaaaaaaaggcattttgatttaggttgctgtgtttgcttgttgataaagaaaactcaatcgggacactagttttgtgctcagctttaggccgggtagctaatgggaggatgtccagcctgtcactgtgctcccagcgcaaggaaatgggtgcccacctggaatcaggagaagaggcttttccctcctgttctgcaaccagggtggagctatctttccagggaagccagctgagaggttttagggctttggttattttatgggggttttaaacctcctaacttttcaatgacaaatggctcccaggtgccatagtctctgttaaatcctcaaacattcacaagcacacactgccaggggcacgggttgtctttcacctgcatgtttctaaggctctttattcaatctcacggtgtcagtgtccagttgtcaaagttatgaatcttcctcctgcttctaaacagggctgacagtatactctcgtctagtctaggaacatgtctgctgctgggataccctggtaccaggatttgagggccacgggtggcatctctgagagctgaaaatccacagagtgcctgtgggaaagccaagcccttggctgtgtggcttttctatcccttggatttacaggtctgggaattggctgcttcttagttataaccccagtgacaaatgctggcttaagccacacctgttcccactgttgctagaattcaaacagttgctttttttttttctttttgagaaagggcctcactctgttgcccaggctggagtgcagtggcttgatcacagctcacgaaagcctcaaactcctaggctcaagtgatcctcctgaaaagtaggtaggactacaggcacatgccaccacatacagctaatttgttttcattttttttttttttagagacaggatctcgctgtgttccccaggtaggtcttgaactcctggcctcaagtgatcctcctgccttgacctcccaaagtgctggattacaagcgtgagcccctgcacccggcccaagcagttgcttctttttttctctttttttttttttttgagatggagcctcactctgttgcccaggctggagtgcagtggcgcgatctccactcactgcaagctccgcctcccgggttcatgccattctcctgcctcagcctcccgagtagctgggactacaggcgcctgccaccacacccagctaattttttgtatttttggtacagacagggtttcaccgtgttagccaggatggtcttgatctcctgatctcgtgatccgcccaccccggcctcccaaagtgctggattacaagcgtgagccaccgcgccccgccaagcagttgcttcttatgcaacatgttggttgggacttgtccacgggccaggccaataaaattcttaatcctgcagagagtcagtaccctcatcaccccatcactggaaaacaaatgttttaagctatcaagagagggaatgtgcagcttttggtttctagatgcatggtttggtgtgatctacctttgtgcctaaagggaatgtcccaaacaacagagccttctttgctgtcactccagaattctctacacagaatttcccaagtccattcaggacagacgcgcagtcctctttcaatggaagaagagaggacttttcccctcctgaaaaatgactggagtgtgaacaaggcagctctgtttttctaaataagttgttcttgtgagttttttctggccactgggcatctctgccctcacttttcatccctgccctctaagctgcagaccccatgaccacactgtctgcttccttgagcttcccgcacgaggcttggacctgggggacctggagaccctgcggacagaactgtggctgagccactgtggccaactcttggggagctccacagtgggggttgctggtctgtgaggctgagtctccatttcagagcacacactccctggcagggcgcctctgcctgtgtctcctgcccagcagccgccagcagggaatagttgctggtgtctgagcacaaagagagctttgattacctagagaggaaaaaggctgtcagccagatgcagccaggcccaggggtagatacaggagttgctaaggaaggggccgagccaggagaggccaggcagatccacaaagcccaaggggatgcaggctgggtgtggtttctgagggaacctaccaaatagcaggtagatggaatcagaggactcttgtgtcctgaaagaacctccttaaaaacaactaaaacgaagaacttctggggctgttcacacattgttcaagtcaccccaagatcgttctggcacgctgagctgaacaccaccatctttgttcattctctctctaatgggcaaagcaggatcatcgagttgaaaagttgtaaataatgaggatatttatcccgctatttattttttcaataactgtgacctcctgcactgtgaatgctctgtgacatgagattcttagtttaataaaactgtcattaaatttgaatgaattgatattattggttactgaacactggcatgagtttatttttattgtgaagaaaaaaatctacagcaatctaaactaaacctttctaagaaatctagcagtcagtattgtaatgcaatatatcaaaatctgtacactgtcaataaaataaatgagcacaaaaaaaaaaaaaa

SEQ ID NO: 65 - DNA, 6840 bp, Homo sapiens
ccggcccagcggctctcctggcctcgcgctgcacattctctcctggcggcggcgccacctgcagtagcgttcgcccgaacatggcgacacggagcagcaggagggagtcgcgactcccgttcctattcaccctggtcgcactgctgccgcccggagctctctgcgaagtctggacgcagaggctgcacggcggcagcgcgcccttgccccaggaccggggcttcctcgtggtgcagggcgacccgcgcgagctgcggctgtgggcgcgcggggatgccaggggggcgagccgcgcggacgagaagccgctccggaggaaacggagcgctgccctgcagcccgagcccatcaaggtgtacggacaggttagtctgaatgattcccacaatcagatggtggtgcactgggctggagagaaaagcaacgtgatcgtggccttggcccgagatagcctggcattggcgaggcccaagagcagtgatgtgtacgtgtcttacgactatggaaaatcattcaagaaaatttcagacaagttaaactttggcttgggaaataggagtgaagctgttatcgcccagttctaccacagccctgcggacaacaagcggtacatctttgcagacgcttatgcccagtacctctggatcacgtttgacttctgcaacactcttcaaggcttttccatcccatttcgggcagctgatctcctcctacacagtaaggcctccaaccttctcttgggctttgacaggtcccaccccaacaagcagctgtggaagtcagatgactttggccagacctggatcatgattcaggaacatgtcaagtccttttcttggggaattgatccctatgacaaaccaaataccatctacattgaacgacacgaaccctctggctactccactgtcttccgaagtacagatttcttccagtcccgggaaaaccaggaagtgatccttgaggaagtgagagattttcagcttcgggacaagtacatgtttgctacaaaggtggtgcatctcttgggcagtgaacagcagtcttctgtccagctctgggtctcctttggccggaagcccatgagagcagcccagtttgtcacaagacatcctattaatgaatattacatcgcagatgcctccgaggaccaggtgtttgtgtgtgtcagccacagtaacaaccgcaccaatttatacatctcagaggcagaggggctgaagttctccctgtccttggagaacgtgctctattacagcccaggaggggccggcagtgacaccttggtgaggtattttgcaaatgaaccatttgctgacttccaccgagtggaaggattgcaaggagtctacattgctactctgattaatggttctatgaatgaggagaacatgagatcggtcatcacctttgacaaagggggaacctgggagtttcttcaggctccagccttcacgggatatggagagaaaatcaattgtgagctttcccagggctgttcccttcatctggctcagcgcctcagtcagctcctcaacctccagctccggagaatgcccatcctgtccaaggagtcggctccaggcctcatcatcgccactggctcagtgggaaagaacttggctagcaagacaaacgtgtacatctctagcagtgctggagccaggtggcgagaggcacttcctggacctcactactacacatggggagaccacggcggaatcatcacggccattgcccagggcatggaaaccaacgagctaaaatacagtaccaatgaaggggagacctggaaaacattcatcttctctgagaagccagtgtttgtgtatggcctcctcacagaacctggggagaagagcactgtcttcaccatctttggctcgaacaaagagaatgtccacagctggctgatcctccaggtcaatgccacggatgccttgggagttccctgcacagagaatgactacaagctgtggtcaccatctgatgagcgggggaatgagtgtttgctgggacacaagactgttttcaaacggcggaccccccatgccacatgcttcaatggagaggactttgacaggccggtggtcgtgtccaactgctcctgcacccgggaggactatgagtgtgacttcggtttcaagatgagtgaagatttgtcattagaggtttgtgttccagatccggaattttctggaaagtcatactcccctcctgtgccttgccctgtgggttctacttacaggagaacgagaggctaccggaagatttctggggacacttgtagcggaggagatgttgaagcgcgactggaaggagagctggtcccctgtcccctggcagaagagaacgagttcattctgtatgctgtgaggaaatccatctaccgctatgacctggcctcgggagccaccgagcagttgcctctcaccgggctacgggcagcagtggccctggactttgactatgagcacaactgtttgtattggtccgacctggccttggacgtcatccagcgcctctgtttgaatggaagcacagggcaagaggtgatcatcaattctggcctggagacagtagaagctttggcttttgaacccctcagccagctgctttactgggtagatgcaggcttcaaaaagattgaggtagctaatccagatggcgacttccgactcacaatcgtcaattcctctgtgcttgatcgtcccagggctctggtcctcgtgccccaagagggggtgatgttctggacagactggggagacctgaagcctgggatttatcggagcaatatggatggttctgctgcctatcacctggtgtctgaggatgtgaagtggcccaatggcatctctgtggacgaccagtggatttactggacggatgcctacctggagtgcatagagcggatcacgttcagtggccagcagcgctctgtcattctggacaacctcccgcacccctatgccattgctgtctttaagaatgaaatctactgggatgactggtcacagctcagcatattccgagcttccaaatacagtgggtcccagatggagattctggcaaaccagctcacggggctcatggacatgaagattttctacaaggggaagaacactggaagcaatgcctgtgtgcccaggccatgcagcctgctgtgcctgcccaaggccaacaacagtagaagctgcaggtgtccagaggatgtgtccagcagtgtgcttccatcaggggacctgatgtgtgactgccctcagggctatcagctcaagaacaatacctgtgtcaaagaagagaacacctgtcttcgcaaccagtatcgctgcagcaacgggaactgtatcaacagcatttggtggtgtgactttgacaacgactgtggagacatgagcgatgagagaaactgccctaccaccatctgtgacctggacacccagtttcgttgccaggagtctgggacttgtatcccactgtcctataaatgtgaccttgaggatgactgtggagacaacagtgatgaaagtcattgtgaaatgcaccagtgccggagtgacgagtacaactgcagttccggcatgtgcatccgctcctcctgggtatgtgacggggacaacgactgcagggactggtctgatgaagccaactgtaccgccatctatcacacctgtgaggcctccaacttccagtgccgaaacgggcactgcatcccccagcggtgggcgtgtgacggggatacggactgccaggatggttccgatgaggatccagtcaactgtgagaagaagtgcaatggattccgctgcccaaacggcacttgcatcccatccagcaaacattgtgatggtctgcgtgattgctctgatggctccgatgaacagcactgcgagcccctctgtacgcacttcatggactttgtgtgtaagaaccgccagcagtgcctgttccactccatggtctgtgacggaatcatccagtgccgcgacgggtccgatgaggatgcggcgtttgcaggatgctcccaagatcctgagttccacaaggtatgtgatgagttcggtttccagtgtcagaatggagtgtgcatcagtttgatttggaagtgcgacgggatggatgattgcggcgattattctgatgaagccaactgcgaaaaccccacagaagccccaaactgctcccgctacttccagtttcggtgtgagaatggccactgcatccccaacagatggaaatgtgacagggagaacgactgtggggactggtctgatgagaaggattgtggagattcacatattcttcccttctcgactcctgggccctccacgtgtctgcccaattactaccgctgcagcagtgggacctgcgtgatggacacctgggtgtgcgacgggtaccgagattgtgcagatggctctgacgaggaagcctgccccttgcttgcaaacgtcactgctgcctccactcccacccaacttgggcgatgtgaccgatttgagttcgaatgccaccaaccgaagacgtgtattcccaactggaagcgctgtgacggccaccaagattgccaggatggccgggacgaggccaattgccccacacacagcaccttgacttgcatgagcagggagttccagtgcgaggacggggaggcctgcattgtgctctcggagcgctgcgacggcttcctggactgctcggacgagagcgatgaaaaggcctgcagtgatgagttgactgtgtacaaagtacagaatcttcagtggacagctgacttctctggggatgtgactttgacctggatgaggcccaaaaaaatgccctctgcatcttgtgtatataatgtctactacagggtggttggagagagcatatggaagactctggagacccacagcaataagacaaacactgtattaaaagtcttgaaaccagataccacgtatcaggttaaagtacaggttcagtgtctcagcaaggcacacaacaccaatgactttgtgaccctgaggaccccagagggattgccagatgcccctcgaaatctccagctgtcactccccagggaagcagaaggtgtgattgtaggccactgggctcctcccatccacacccatggcctcatccgtgagtacattgtagaatacagcaggagtggttccaagatgtgggcctcccagagggctgctagtaactttacagaaatcaagaacttattggtcaacactctatacaccgtcagagtggctgcggtgactagtcgtggaataggaaactggagcgattctaaatccattaccaccataaaaggaaaagtgatcccaccaccagatatccacattgacagctatggtgaaaattatctaagcttcaccctgaccatggagagtgatatcaaggtgaatggctatgtggtgaaccttttctgggcatttgacacccacaagcaagagaggagaactttgaacttccgaggaagcatattgtcacacaaagttggcaatctgacagctcatacatcctatgagatttctgcctgggccaagactgacttgggggatagccctctggcatttgagcatgttatgaccagaggggttcgcccacctgcacctagcctcaaggccaaagccatcaaccagactgcagtggaatgtacctggaccggcccccggaatgtggtttatggtattttctatgccacgtcctttcttgacctctatcgcaacccgaagagcttgactacttcactccacaacaagacggtcattgtcagtaaggatgagcagtatttgtttctggtccgtgtagtggtaccctaccaggggccatcctctgactacgttgtagtgaagatgatcccggacagcaggcttccaccccgtcacctgcatgtggttcatacgggcaaaacctccgtggtcatcaagtgggaatcaccgtatgactctcctgaccaggacttgttgtatgcaattgcagtcaaagatctcataagaaagactgacaggagctacaaagtaaaatcccgtaacagcactgtggaatacacccttaacaagttggagcctggcgggaaataccacatcattgtccaactggggaacatgagcaaagattccagcataaaaattaccacagtttcattatcagcacctgatgccttaaaaatcataacagaaaatgatcatgttcttctgttttggaaaagcctggctttaaaggaaaagcattttaatgaaagcaggggctatgagatacacatgtttgatagtgccatgaatatcacagcttaccttgggaatactactgacaatttctttaaaatttccaacctgaagatgggtcataattacacgttcaccgtccaagcaagatgcctttttggcaaccagatctgtggggagcctgccatcctgctgtacgatgagctggggtctggtgcagatgcatctgcaacgcaggctgccagatctacggatgttgctgctgtggtggtgcccatcttattcctgatactgctgagcctgggggtggggtttgccatcctgtacacgaagcaccggaggctgcagagcagcttcaccgccttcgccaacagccactacagctccaggctggggtccgcaatcttctcctctggggatgacctgggggaagatgatgaagatgcccctatgataactggattttcagatgacgtccccatggtgatagcctgaaagagctttcctcactagaaaccaaatggtgtaaatattttatttgataaagatagttgatggtttattttaaaagatgcactttgagttgcaatatgttatttttatatgggcc

SEQ ID NO: 66 - DNA, 1364 bp, Homo sapiens
aaaatcaggaacttgtgctggccctgcaatgtcaagggagggggctcacccagggctcctgtagctcagggggcaggcctgagccctgcacccgccccacgaccgtccagcccctgacgggcaccccatcctgaggggctctgcattggcccccaccgaggcaggggatctgaccgactcggagcccggctggatgttacaggcgtgcaaaatggaagggtttcccctcgtcccccctccatcagaagacctggtgccctatgacacggatctataccaacgccaaacgcacgagtattacccctatctcagcagtgatggggagagccatagcgaccattactgggacttccacccccaccacgtgcacagcgagttcgagagcttcgccgagaacaacttcacggagctccagagcgtgcagcccccgcagctgcagcagctctaccgccacatggagctggagcagatgcacgtcctcgatacccccatggtgccaccccatcccagtcttggccaccaggtctcctacctgccccggatgtgcctccagtacccatccctgtccccagcccagcccagctcagatgaggaggagggcgagcggcagagccccccactggaggtgtctgacggcgaggcggatggcctggagcccgggcctgggctcctgcctggggagacaggcagcaagaagaagatccgcctgtaccagttcctgttggacctgctccgcagcggcgacatgaaggacagcatctggtgggtggacaaggacaagggcaccttccagttctcgtccaagcacaaggaggcgctggcgcaccgctggggcatccagaagggcaaccgcaagaagatgacctaccagaagatggcgcgcgcgctgcgcaactacggcaagacgggcgaggtcaagaaggtgaagaagaagctcacctaccagttcagcggcgaagtgctgggccgcgggggcctggccgagcggcgccacccgccccactgagcccgcagcccccgccggccccgccaggcctccccgctggccatagcattaagccctcgcccggcccggacacagggaggacgctcccggggcccagaggcaggactgtggcgggccgggctccgtcacccgcccctccccccactccaggccccctccacatcccgcttcgcctccctccaggactccaccccggctcccgacgccagctgggcgtcagacccaccggcaaccttgcagaggacgacccggggtactgccttgggagtctcaagtccgtatgtaaatcagatctcccctctcacccctcccacccattaacctcctcccaaaaaacaagtaaagttattctcaatcc

SEQ ID NO: 67 - DNA, 1641 bp, Homo sapiens
ctccctgtgttggtggaggatgtctgcagcagcatttaaattctgggagggcttggttgtcagcagcagcaggaggaggcagagcacagcatcgtcgggaccagactcgtctcaggccagttgcagccttctcagccaaacgccgaccaaggaaaactcactaccatgagaattgcagtgatttgcttttgcctcctaggcatcacctgtgccataccagttaaacaggctgattctggaagttctgaggaaaagcagctttacaacaaatacccagatgctgtggccacatggctaaaccctgacccatctcagaagcagaatctcctagccccacagaatgctgtgtcctctgaagaaaccaatgactttaaacaagagacccttccaagtaagtccaacgaaagccatgaccacatggatgatatggatgatgaagatgatgatgaccatgtggacagccaggactccattgactcgaacgactctgatgatgtagatgacactgatgattctcaccagtctgatgagtctcaccattctgatgaatctgatgaactggtcactgattttcccacggacctgccagcaaccgaagttttcactccagttgtccccacagtagacacatatgatggccgaggtgatagtgtggtttatggactgaggtcaaaatctaagaagtttcgcagacctgacatccagtaccctgatgctacagacgaggacatcacctcacacatggaaagcgaggagttgaatggtgcatacaaggccatccccgttgcccaggacctgaacgcgccttctgattgggacagccgtgggaaggacagttatgaaacgagtcagctggatgaccagagtgctgaaacccacagccacaagcagtccagattatataagcggaaagccaatgatgagagcaatgagcattccgatgtgattgatagtcaggaactttccaaagtcagccgtgaattccacagccatgaatttcacagccatgaagatatgctggttgtagaccccaaaagtaaggaagaagataaacacctgaaatttcgtatttctcatgaattagatagtgcatcttctgaggtcaattaaaaggagaaaaaatacaatttctcactttgcatttagtcaaaagaaaaaatgctttatagcaaaatgaaagagaacatgaaatgcttctttctcagtttattggttgaatgtgtatctatttgagtctggaaataactaatgtgtttgataattagtttagtttgtggcttcatggaaactccctgtaaactaaaagcttcagggttatgtctatgttcattctatagaagaaatgcaaactatcactgtattttaatatttgttattctctcatgaatagaaatttatgtagaagcaaacaaaatacttttacccacttaaaaagagaatataacattttatgtcactataatcttttgttttttaagttagtgtatattttgttgtgattatctttttgtggtgtgaataaatcttttatcttgaatgtaataagaatttggtggtgtcaattgcttatttgttttcccacggttgtccagcaattaataaaacataaccttttttactgcctaaaaaaaaaaaaaaaaa

SEQ ID NO: 68 - DNA, 2010 bp, Homo sapiens
aagaggaagtcgcgctgctgtggcggccgctgtagcagcggcggtccagtcgtagcccggccgcccgcgcctgtccggtccggtccggccacggaggcagcgcagcggcgggactccgagcctaccccgccgagtgagctgcgccgcaccgtgccgtcccacccggcacccaccagtccgatggggccgcagcggcggctgtcccctgccggggccgccctactctggggcttcctgctccagctgacagccgctcaggaagcaatcttgcatgcgtctggaaatggcacaaccaaggactactgcatgctttataacccttattggacagctcttccaagtaccctagaaaatgcaacttccattagtttgatgaatctgacttccacaccactatgcaacctttctgatattcctcctgttggcataaagagcaaagcagttgtggttccatggggaagctgccattttcttgaaaaagccagaattgcacagaaaggaggtgctgaagcaatgttagttgtcaataacagtgtcctatttcctccctcaggtaacagatctgaatttcctgatgtgaaaatactgattgcatttataagctacaaagactttagagatatgaaccagactctaggagataacattactgtgaaaatgtattctccatcgtggcctaactttgattatactatggtggttatttttgtaattgcggtgttcactgtggcattaggtggatactggagtggactagttgaattggaaaacttgaaagcagtgacaactgaagatagagaaatgaggaaaaagaaggaagaatatttaacttttagtcctcttacagttgtaatatttgtggtcatctgctgtgttatgatggtcttactttatttcttctacaaatggttggtttatgttatgatagcaattttctgcatagcatcagcaatgagtctgtacaactgtcttgctgcactaattcataagataccatatggacaatgcacgattgcatgtcgtggcaaaaacatggaagtgagacttatttttctctctggactgtgcatagcagtagctgttgtttgggctgtgtttcgaaatgaagacaggtgggcttggattttacaggatatcttggggattgctttctgtctgaatttaattaaaacactgaagttgcccaacttcaagtcatgtgtgatacttctaggccttctcctcctctatgatgtattttttgttttcataacaccattcatcacaaagaatggtgagagtatcatggttgaactcgcagctggaccttttggaaataatgaaaagttgccagtagtcatcagagtaccaaaactgatctatttctcagtaatgagtgtgtgcctcatgcctgtttcaatattgggttttggagacattattgtaccaggcctgttgattgcatactgtagaagatttgatgttcagactggttcttcttacatatactatgtttcgtctacagttgcctatgctattggcatgatacttacatttgttgttctggtgctgatgaaaaaggggcaacctgctctcctctatttagtaccttgcacacttattactgcctcagttgttgcctggagacgtaaggaaatgaaaaagttctggaaaggtaacagctatcagatgatggaccatttggattgtgcaacaaatgaagaaaaccctgtgatatctggtgaacagattgtccagcaataatattatgtggaactgctataatgtgtcattgattttctacaaatagacttcgactttttaaattgacttttgaattgacaatctgaaagagtcttcaatgatatgcttgcaaaaatatatttttatgagctggtactgacagttacatcataaataactaaaacgctttgcttttaatgttaaagttgtgccttcacattaaataaaacatatggtctgtgtagtttccgagatgtactatatacagtatatttttctaaaaaaaaa

SEQ ID NO: 69 - DNA, 2827 bp, Homo sapiens
gccggcggtggcgcggcggagacccggctggtataacaagaggattgcctgatccagccaagatgcagagcacttctaatcatctgtggcttttatctgatattttaggccaaggagctactgcaaatgtctttcgtggaagacataagaaaactggtgatttatttgctatcaaagtatttaataacataagcttccttcgtccagtggatgttcaaatgagagaatttgaagtgttgaaaaaactcaatcacaaaaatattgtcaaattatttgctattgaagaggagacaacaacaagacataaagtacttattatggaattttgtccatgtgggagtttatacactgttttagaagaaccttctaatgcctatggactaccagaatctgaattcttaattgttttgcgagatgtggtgggtggaatgaatcatctacgagagaatggtatagtgcaccgtgatatcaagccaggaaatatcatgcgtgttataggggaagatggacagtctgtgtacaaactcacagattttggtgcagctagagaattagaagatgatgagcagtttgtttctctgtatggcacagaagaatatttgcaccctgatatgtatgagagagcagtgctaagaaaagatcatcagaagaaatatggagcaacagttgatctttggagcattggggtaacattttaccatgcagctactggatcactgccatttagaccctttgaagggcctcgtaggaataaagaagtgatgtataaaataattacaggaaagccttctggtgcaatatctggagtacagaaagcagaaaatggaccaattgactggagtggagacatgcctgtttcttgcagtctttctcggggtcttcaggttctacttacccctgttcttgcaaacatccttgaagcagatcaggaaaagtgttggggttttgaccagttttttgcagaaactagtgatatacttcaccgaatggtaattcatgttttttcgctacaacaaatgacagctcataagatttatattcatagctataatactgctactatatttcatgaactggtatataaacaaaccaaaattatttcttcaaatcaagaacttatctacgaagggcgacgcttagtcttagaacctggaaggctggcacaacatttccctaaaactactgaggaaaaccctatatttgtagtaagccgggaacctctgaataccataggattaatatatgaaaaaatttccctccctaaagtacatccacgttatgatttagacggggatgctagcatggctaaggcaataacaggggttgtgtgttatgcctgcagaattgccagtaccttactgctttatcaggaattaatgcgaaaggggatacgatggctgattgaattaattaaagatgattacaatgaaactgttcacaaaaagacagaagttgtgatcacattggatttctgtatcagaaacattgaaaaaactgtgaaagtatatgaaaagttgatgaagatcaacctggaagcggcagagttaggtgaaatttcagacatacacaccaaattgttgagactttccagttctcagggaacaatagaaaccagtcttcaggatatcgacagcagattatctccaggtggatcactggcagacgcatgggcacatcaagaaggcactcatccgaaagacagaaatgtagaaaaactacaagtcctgttaaattgcatgacagagatttactatcagttcaaaaaagacaaagcagaacgtagattagcttataatgaagaacaaatccacaaatttgataagcaaaaactgtattaccatgccacaaaagctatgacgcactttacagatgaatgtgttaaaaagtatgaggcatttttgaataagtcagaagaatggataagaaagatgcttcatcttaggaaacagttattatcgctgactaatcagtgttttgatattgaagaagaagtatcaaaatatcaagaatatactaatgagttacaagaaactctgcctcagaaaatgtttacagcttccagtggaatcaaacataccatgaccccaatttatccaagttctaacacattagtagaaatgactcttggtatgaagaaattaaaggaagagatggaaggggtggttaaagaacttgctgaaaataaccacattttagaaaggtttggctctttaaccatggatggtggccttcgcaacgttgactgtctttagctttctaatagaagtttaagaaaagtttccgtttgcacaagaaaataacgcttgggcattaaatgaatgcctttatagatagtcacttgtttctacaattcagtatttgatgtggtcgtgtaaatatgtacaatattgtaaatacataaaaaatatacaaatttttggctgctgtgaagatgtaattttatcttttaacatttataattatatgaggaaatttgacctcagtgatcacgagaagaaagccatgaccgaccaatatgttgacatactgatcctctactctgagtggggctaaataagttattttctctgaccgcctactggaaatatttttaagtggaaccaaaataggcatccttacaaatcaggaagactgacttgacacgtttgtaaatggtagaacggtggctactgtgagtggggagcagaaccgcaccactgttatactgggataacaatttttttgagaaggataaagtggcattattttattttacaaggtgcccagatcccagttatccttgtatccatgtaatttcagatgaattattaagcaaacattttaaagt

SEQ ID NO: 70 - DNA, 1643 bp, Homo sapiens
gcagaggaccagctaagagggagagaagcaactacagaccccccctgaaaacaaccctcagacgccacatcccctgacaagctgccaggcaggttctcttcctctcacatactgacccacggctccaccctctctcccctggaaaggacaccatgagcactgaaagcatgatccgggacgtggagctggccgaggaggcgctccccaagaagacaggggggccccagggctccaggcggtgcttgttcctcagcctcttctccttcctgatcgtggcaggcgccaccacgctcttctgcctgctgcactttggagtgatcggcccccagagggaagagttccccagggacctctctctaatcagccctctggcccaggcagtcagatcatcttctcgaaccccgagtgacaagcctgtagcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcctaccagaccaaggtcaacctcctctctgccatcaagagcccctgccagagggagaccccagagggggctgaggccaagccctggtatgagcccatctatctgggaggggtcttccagctggagaagggtgaccgactcagcgctgagatcaatcggcccgactatctcgactttgccgagtctgggcaggtctactttgggatcattgccctgtgaggaggacgaacatccaaccttcccaaacgcctcccctgccccaatccctttattaccccctccttcagacaccctcaacctcttctggctcaaaaagagaattgggggcttagggtcggaacccaagcttagaactttaagcaacaagaccaccacttcgaaacctgggattcaggaatgtgtggcctgcacagtgaattgctggcaaccactaagaattcaaactggggcctccagaactcactggggcctacagctttgatccctgacatctggaatctggagaccagggagcctttggttctggccagaatgctgcaggacttgagaagacctcacctagaaattgacacaagtggaccttaggccttcctctctccagatgtttccagacttccttgagacacggagcccagccctccccatggagccagctccctctatttatgtttgcacttgtgattatttattatttatttattatttatttatttacagatgaatgtatttatttgggagaccggggtatcctgggggacccaatgtaggagctgccttggctcagacatgttttccgtgaaaacggagctgaacaataggctgttcccatgtagccccctggcctctgtgccttcttttgattatgttttttaaaatatttatctgattaagttgtctaaacaatgctgatttggtgaccaactgtcactcattgctgagcctctgctccccaggggagttgtgtctgtaatcgccctactattcagtggcgagaaataaagtttgctt

SEQ ID NO: 71 - DNA, 1041 bp, Homo sapiens
tgacatgcctgatcctctcttttctgcagttcaagggaaagacgagatcttgcacaaggcactctgcttctgcccttggctggggaagggtggcatggagcctctccggctgctcatcttactctttgtcacagagctgtccggagcccacaacaccacagtgttccagggcgtggcgggccagtccctgcaggtgtcttgcccctatgactccatgaagcactgggggaggcgcaaggcctggtgccgccagctgggagagaagggcccatgccagcgtgtggtcagcacgcacaacttgtggctgctgtccttcctgaggaggtggaatgggagcacagccatcacagacgataccctgggtggcactctcaccattacgctgcggaatctacaaccccatgatgcgggtctctaccagtgccagagcctccatggcagtgaggctgacaccctcaggaaggtcctggtggaggtgctggcagaccccctggatcaccgggatgctggagatctctggttccccggggagtctgagagcttcgaggatgcccatgtggagcacagcatctccaggagcctcttggaaggagaaatccccttcccacccacttccatccttctcctcctggcctgcatctttctcatcaagattctagcagccagcgccctctgggctgcagcctggcatggacagaagccagggacacatccacccagtgaactggactgtggccatgacccagggtatcagctccaaactctgccagggctgagagacacgtgaaggaagatgatgggaggaaaagcccaggagaagtcccaccagggaccagcccagcctgcatacttgccacttggccaccaggactccttgttctgctctggcaagagactactctgcctgaacactgcttctcctggaccctggaagcagggactggttgagggagtggggaggtggtaagaacacctgacaacttctgaatattggacattttaaacacttacaaataaatccaagactgtcatatttaaaaa

SEQ ID NO: 72 - DNA, 3840 bp, Homo sapiens
caatgaatccctgcggttggctgggggcagtgggtcccacactgcctcacttccctaaatgggcagcttcacttttagaaccccgggtccttccctggcaggcccaggtggcacatcctgtgtcgggtgggccctcaccttggatctccaggcctgacactgcccagctggatggaaccatggccccagccttcctgctgctgctgctgctgtggccacagggttgcgtctcaggcccctctgctgacagtgtatacacaaaagtgaggctccttgaaggggagactctgtctgtgcagtgctcctataagggctacaaaaaccgcgtggagggcaaggtttggtgcaaaatcaggaagaagaagtgtgagcctggctttgcccgagtctgggtgaaagggccccgctacttgctgcaggacgatgcccaggccaaggtggtcaacatcaccatggtggccctcaagctccaggactcaggccgatactggtgcatgcgcaacacctctgggatcctgtaccccttgatgggcttccagctggatgtgtctccagctccccaaactgagaggaacattcctttcacacatctggacaacatcctcaagagtggaactgtcacaactggccaagcccctacctcaggccctgatgccccttttaccactggtgtgatggtgttcaccccaggactcatcaccttgcctaggctcttagcctccaccagacctgcctccaagacaggctacagcttcactgctaccagcaccaccagccagggacccaggaggaccatggggtcccagacagtgaccgcgtctcccagcaatgccagggactcctctgctggcccagaatccatctccactaagtctggggacctcagcaccagatcgcccaccacagggctctgcctcaccagcagatctctcctcaacagactaccctccatgccctccatcaggcaccaggatgtttactccactgtgcttggggtggtgctgaccctcctggtgctgatgctgatcatggtctatgggttttggaagaagagacacatggcaagctacagcatgtgcagcgatccttctacacgtgacccacctggaagaccagagccctatgtggaagtctacttgatctgaggccacttaagcatggggtggggagcttctcccagagtggccccagggggttagaggaggggtgaagattggggccagtatcgatcttatgaagctggaggacttgtgcagtgctggactcacccaggacttcccaaacccagaggctgccatcctaagcagccccacagcccagtgttctccttgggggcaggaacctggggaggggcccagagcaaagggcatcagggagaaagtcccgaggaaatgtgaccagtggtttctgctcggagctgcagaccccagggctcttggtggaggcaggggaaccctgagagtgctgtttacagagaacctcagctcccgtctgcctcagaaaccctattgggctgagctgccctccccaccagggccactgtgtcctctgcttccctccgttctgcttcagcttcccctaaggttagggaagaaagaatcgggctcacgaatgccagaggcagtgatgtcccatcctggaggagaggaaacagtgactaaaagctggggacccacagaggggttggcagcttctcttgtcgggacaggtgtcctttgctgggcctctggatggccctgccctgactggggctgctcctccctcctgtcctgggaccgcgcagagcccacgctctcactgctgcctcctgctggccgctgcctccttagaaagctgtgaccaggcagctaagagcctctgggctgcagggtcagcctctcccaagactgaagtgcagaggctggacttggggctctctcccccagcttctacacctgggctccaagtctgagttcccacaggggacccagcagcctccaggaagtccataccctggggtggctgagaccttggctctgtatggaggctgctcaccccacagacactggtggggagaccatggctcagaggaagggtggagcaaccctcctcctacccctcaggatagagagagaagacacacttgggacacagtgaagacagtaacttggaactgaccacggcctggaggactggcccaggcagggggacagggaaaatggagcccaagtagcctctggccagggacccaatgtcccgaggaatctgcctcccacccactgactcagggctcagactcagcctctattgtccagagcactggcttggcgtccagcaatgaaggctggagaatgcagcctggattcccctacacacacacacacacacacacacacacacacacacacacacacacacacacaggtgtctactgacctggagtgactggaatagcacctggggataaatgtgacaactgtgcattgaaccctgggtcagggacgttccaatggccaagagagtgacacagccaggaccctggtggacagccagaggggccacttcaggatggatgtggggagagtggaagaggcagggagtaatcctgggggacagcagggaggaggcacttcttccctatgtccaggagagggcaatagagggaagactgaggctgaagaattgacggctctggacccaggacagacagacagacagacagacagacagacagacagacacgcacacacacccatctctgtctagcaagcagcctcctaagatagctgttctccctatcatgacggtgtagccaccatcctgttgtatactaggagagaacttaacccacctgggggaaaatagctccccaagagctggcaccagtaccactgatggccctgcttcctctgagtgagatgcccaggaggaggagccctagggaagaagtcagggacagggaccaggataccactctgtcactgtgtgaccctcagcaagtcactaacccttggcctcatttttcctgtcttgtgaaagaggacaataattcctacttctcaagattgttttcaagataaaataacattagcattgtacaatgatgcaaatgcctcattaccattattccttaagttgttttccagctctaatgttgtttccaacattacatttaagaccttaggattctgtttcttgcttttgtcatatctcttcccaagtgtcatcactatatggatgttgagggcccccgatgacagtccctttggtaaggtcctcttttgaggaggggagggtacagggtggactcatctcagtgtgaacttggcaagtcactgtccctctctgatcttgtttcctcatctggagaaggagtgagagaggagaaaggaagaaaccagtcaggcaggcagttagggtgggttctcggtagaattcttttaaacaaaagaacagcctgaaaaatcaagctgcaggcacagatatgggaacttgcacaggggggcttgcctaagacatgcccacagcctcatagataagacagactacacaggtgacttgcccaaacatgcctgcaatggaaaatttcatcccctgacatgtgcagtaaggggaacaaagcaatatggagtaagtaactcaagccaagggcccacatgtacattagaaggacagcagggagctaccagaaattcatgccttatgcagatgagctgcccagtcctcatcggtttcttataaaagcctttacattcaactgtaaaaatggcaaccctctttcaggcctcctctccacagcagagagctttcttctctcactcattaaactttcactccaacctcaaaaaaaaaaaaaaaaaa

SEQ ID NO: 73 - DNA, 604 bp, Homo sapiens
ccacgcgtccgcgctgcgccacatcccaccggcccttacactgtggtgtccagcagcatccggcttcatggggggacttgaaccctgcagcaggctcctgctcctgcctctcctgctggctgtaagtggtctccgtcctgtccaggcccaggcccagagcgattgcagttgctctacggtgagcccgggcgtgctggcagggatcgtgatgggagacctggtgctgacagtgctcattgccctggccgtgtacttcctgggccggctggtccctcgggggcgaggggctgcggaggcagcgacccggaaacagcgtatcactgagaccgagtcgccttatcaggagctccagggtcagaggtcggatgtctacagcgacctcaacacacagaggccgtattacaaatgagcccgaatcatgacagtcagcaacatgatacctggatccagccattcctgaagcccaccctgcacctcattccaactcctaccgcgatacagacccacagagtgccatccctgagagaccagaccgctccccaatactctcctaaaataaacatgaagcacaaaaaaaaaaaaaaaaaaaaaaaaaaaaaaaaaa

SEQ ID NO: 74 - DNA, 2357 bp, Homo sapiens
cgccgttttcccggggagatgcgccgcccggtctccctgccagcggagtgctgggccgaggacagggcggcaggggtgacagtggggtccaggagagtctcaaaatcctaagctttcagtatttgttattgtgaaagaagttaattcacctgaaacagaggaggggcaacctgagttatcagaaagtgacttcctggccttcccttctttactgatcagaggcacacaaagcgtagtttctaagctgaatgatgacaacgttgcagaataaagaagaatgtggaaagggaccaaagagaatctttgccccacctgcacaaaaatcttacagcctgttaccttgtagccctaactcccctaaggaggagaccccggggatcagttccccagagacagaggccaggataagcctgccaaaggccagtttaaagaagaaagaggaaaaagcaaccatgaagaatgttccaagcagggaacaggagaaaaaaagaaaggcacaaatcaacaagcaagcagagaagaaagaaaaggaaaaatcaagtcttaccaatgcagaatttgaggagattgtccagattgttctgcagaagtcccttcaggagtgcttggggatgggatctggccttgattttgcagagacttcttgtgcccagcccgtagtatctacccaatcagacaaggagccaggaattactgcttctgctactgatactgataatgctaatggagaggaggtaccacatactcaagagatttcagtgtcttgggaaggtgaagctgcccctgagataaggacatctaagttaggccagccagatcctgcaccctctaagaagaaatccaatagactcaccttaagcaaaagaaagaaggaagctcatgagaaggtggagaaaactcaaggtggacatgagcacagacaggaagaccgactaaagaaaacagttcaggatcattctcagatcagggaccagcaaaaaggagagataagtggttttggtcaatgtctggtctgggtccagtgttccttcccaaactgtgggaaatggaggcggctgtgtgggaacattgacccctcagttctcccagataattggtcctgtgatcagaacacagatgtgcagtataatcgctgtgatattcctgaggagacctggacagggcttgagagtgatgtggcctatgcctcctacatcccaggatccatcatctgggccaagcaatacggttacccctggtggccaggcatgatagaatctgatcctgacttaggggaatattttctttttacttcccatcttgattccctgccgtctaagtaccatgtgacgttttttggagaaacagtttctcgtgcatggatcccagtcaacatgctaaagaacttccaggagctgtccctggagctatcagtcatgaaaaagcgcagaaatgactgcagccagaaactgggggtggccctgatgatggctcaagaggcagaacagatcagcattcaggaacgggttaacttgtttggtttctggagccgattcaacggatctaacagtaatggggaaagaaaagacttacagctctctggtttgaacagcccaggatcctgcttagagaaaaaggagaaagaggaagagttggaaaaggaggaaggagagaaaacagacccaattttgcccattcgtaagcgagtcaaaatacagacccaaaaaaaccaagccaagagggcttgggggtgatgcaggcacagcagatggccgaggcaggacactgcagaggaagataatgaagagatctctaggcaggaaatccacagctcctcctgcacccagaatgggaaggaaagaaggccaagggaattcagattctgaccagccaggccctaagaaaaaatttaaagctccccagagcaaggccttggcagccagcttttcagagggaaaagaagttagaacagtgccaaagaacctgggcctatcagcgtgtaagggggcctgcccctcatctgcgaaagaagagcccagacaccgggaacccctgacccaggaggctggaagtgtcccccttgaggacgaagcctccagtgacctggacctggagcaactcatggaagatgttgggagagagctggggcagagcggggagctgcagcacagcaacagtgatggcgaggacttccccgtggcgctgtttgggaagtagctggtgctcctctgctccctctttttctcccttctctggggcgcaggagggagaagttgctaagtgctgggtctgttcattggctatgaggttcaaatgtgtgtggtgcagtttctgtgttaataaagcaggttacagtcgaaaaaaaaaaaaaaaaaaaa